SEQUENCE LISTING (84 sequences)

SEQ ID NO: 1 - DNA, 270 bp, Canus lupus familiaris
cacatttcgtgtaccagtttaagcccgagtgccatttcaccaacgggacggagcgggtgcggttcgtggaaagacacatccataaccgggaggagttcgtgcgcttcgacagcgacgtgggggagtaccgggcggtcacggagctcgggcggcccgacgctgagtcctggaacgggcagaaggagctcttggagcaggagcgggcaacggtggacacctactgcagacacaactacggggtgattgagagcttcacggtgcagcggcgag

SEQ ID NO: 2 - DNA, 270 bp, Canus lupus familiaris
cacatttcttggaggtggcaaaggccgagtgccatttcaccaacgggacggagcgggtgcggttcgtggaaagatacatctataaccgggaggagtacgtgcgcttcgacagcgacgtgggggagtaccgggcggtcacggagctcgggcggcccgacgctgagtactggaacccgcagaaggagctcttggagcgggcgcgggccgcggtggacacctactgcagacacaactacggggtgggcgagagcttcacggtgcagcggcgag

SEQ ID NO: 3 - DNA, 270 bp, Canus lupus familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacggtgtgcagacacaactacggggtgattgagagcttcacggtgcagcggcgag

SEQ ID NO: 4 - DNA, 270 bp, Canus lupus familiaris
cacatttcttggagatggtaaagttcgagtgccatttcaccaacgggacggagcgggtgcggcttctggtgagagacatctataaccgggaggagcacgtgcgcttcgacagcgacgtgggggagtaccgggcggtcacggagctcgggcggcccgacgctgagtactggaacgggcagaaggagctcttggagcagaggcgggccgaggtggacacggtgtgcagacacaactacggggtgattgagagcttcgcggtgcagcggcgag

SEQ ID NO: 5 - DNA, 270 bp, Canus lupus familiaris
cacatttcttggagatgttaaagttcgagtgccatttcaccaacgggacggagcgggtgcggttcgtggaaagatacatccataaccgggaggagttcgtgcgcttcgacagcgacgtgggggagtaccgggcggtcacggagctcgggcggcccgacgctgagtcctggaaccggcagaaggagctcttggagcaggagcgggccgcggtggacacctactgcagacacaactaccgggtgggcgagagcttcacggtgcagcggcgag

SEQ ID NO: 6 - DNA, 267 bp, Canus lupus familiaris
gatttcgtgtaccagtttaaggccgagtgctatttcaccaacgggacggagcgggtgcggcttctgactaaatacatctataaccgggaggagtacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 7 - DNA, 267 bp, Canus lupus familiaris
gatttcgtgttccagtataaggccgagtgctatttcaccaacgggacggagcgggtgcggcttctgactaaatacatctataaccgggaggagtacgtgcgcttcgacagcgacgtgggggagtaccgggcggtcacggagctcgggcggccctgggctgagtactggaacccgcagaaggacgagatggaccgggtacgggccgagctggacacggtgtgcagacacaactacgggttggaagagctcaccacgttgcagcggcga

SEQ ID NO: 8 - DNA, 267 bp, Canus lupus familiaris
gatttcgtgtaccagtgtaaggccgagtgctatttcaccaacgggacggagcgggtgcggtttctggctaaatacatctataaccgggaggagttcgtgcgcttcgacagcgacgtgggggagttccgggcggtcacggagctcgggcggccctcggctgagtactggaacgggcagaaggagatcttggagcaggagcgggcaacggtggacacggtgtgcagacacaactacggggtggaagagctctacacgttgcagcggcga

SEQ ID NO: 9 - DNA, 267 bp, Canus lupus familiaris
gatttcgtgtaccagtttaaggccgagtgctatttcaccaacgggacggagcgggtgcggcttctgacgagaagcatctataaccgggaggagttcgtgcgcttcgacagcgacgtgggggagttccgggcggtcacggagctcgggcggcccgtcgctgagtactggaacgggcagaaggagatcttggagcggaagcgggccgcggtggacacggtgtgcagacacaactacgggagggaagagctcaccacgttgcagcggcga

SEQ ID NO: 10 - DNA, 267 bp, Canus lupus familiaris
gatttcgtgtaccagtttaagggcgagtgctatttcaccaacgggacggagcgggtgcggcttctgactaaatacatctataaccgggaggagtacgtgcgcttcgacagcgacgtgggggagtaccgggcggtcacggagctcgggcggccctcggctgagtactggaacccgcagaaggacgagatggaccgggtacgggccgagctggacacggtgtgcagacacaactacgggttggaagagctcaccacgttgcagcggcga

SEQ ID NO: 11 - DNA, 246 bp, Canus lupus familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggctataacaaaacaaaacttgaacatcatgactaaaaggtccaacaaaactgctgctaccaat

SEQ ID NO: 12 - DNA, 246 bp, Canus lupus familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttacaagttttgacccacagggtgcactgagaaacttggccagagcaaaacaaaacttgaacatcctgactaaaagttccaaccaaactgctgctaccaat

SEQ ID NO: 13 - DNA, 246 bp, Canus lupus familiaris
gaccatgttgcctactacggcataaatgtctaccagtcttacggtccctctggccagttcacccatgaatttgatggcgatgaggagttctacgtggacctggagaagaaggaaactgtctggcggctgcctgtgtttagcacatttacaagttttgacccacagggtgcactgagaaacttggctataacaaaacaaaacttgaacatcatgactaaaaggtccaacaaaactgctgctaccaat

SEQ ID NO: 14 - DNA, 246 bp, Canus lupus familiaris
gaccatgttgcctactacggcataaatgtctaccagtcttacggtccctctggccagtacacccatgaatttgatggcgatgaggagttctacgtggacctggagaagaaggaaactgtctggcggctgcctgtgtttagcacatttagaagttttgacccacagggtgcactgagaaacttggctataataaaacaaaacttgaacatcctgactaaaaggtccaaccaaactgctgctaccaat

SEQ ID NO: 15 - DNA, 22 bp, Artificial Sequence
  Primer
gatggattgctagcagaatgaa

SEQ ID NO: 16 - DNA, 41 bp, Artificial Sequence
  Primer
cacgacgttgtaaaacgacgtagtcgctaatcacgatctat

SEQ ID NO: 17 - DNA, 41 bp, Artificial Sequence
  Primer
cacgacgttgtaaaacgacctgccctgcgttcctctatccc

SEQ ID NO: 18 - DNA, 20 bp, Artificial Sequence
  Primer
ctgaaaactcgcccgaacct

SEQ ID NO: 19 - DNA, 22 bp, Artificial Sequence
  Primer
tgacatcaaatccgacgatgag

SEQ ID NO: 20 - DNA, 40 bp, Artificial Sequence
  Primer
cacgacgttgtaaaacgacgatcagcaccgtcccgctttc

SEQ ID NO: 21 - DNA, 22 bp, Artificial Sequence
  Primer
cagtcgcggtcgcttctcatct

SEQ ID NO: 22 - DNA, 40 bp, Artificial Sequence
  Primer
cacgacgttgtaaaacgacgcagctgcagagctttatgac

SEQ ID NO: 23 - DNA, 22 bp, Artificial Sequence
  Primer
ctatttttgacaataaagcatc

SEQ ID NO: 24 - DNA, 41 bp, Artificial Sequence
  Primer
cacgacgttgtaaaacgactacaattaaggaaacgaattgc

SEQ ID NO: 25 - DNA, 18 bp, Artificial Sequence
  Primer
ttcctcaggttgagggtt

SEQ ID NO: 26 - DNA, 41 bp, Artificial Sequence
  Primer
cacgacgttgtaaaacgacgcttcaatgtactcttgtagtt

SEQ ID NO: 27 - DNA, 41 bp, Artificial Sequence
  Primer
cacgacgttgtaaaacgacattaagaatagatcctcctaca

SEQ ID NO: 28 - DNA, 22 bp, Artificial Sequence
  Primer
actatctactggcaggtatcca

SEQ ID NO: 29 - DNA, 41 bp, Artificial Sequence
  Primer
cacgacgttgtaaaacgactgaggtcgaaggaggagagatg

SEQ ID NO: 30 - DNA, 22 bp, Artificial Sequence
  Primer
atccctagcatactagactttc

SEQ ID NO: 31 - DNA, 22 bp, Artificial Sequence
  Primer
gaaagattctaaatccttgaac

SEQ ID NO: 32 - DNA, 39 bp, Artificial Sequence
  Primer
cacgacgttgtaaaacgactctaatagcatcatttatca

SEQ ID NO: 33 - DNA, 22 bp, Artificial Sequence
  Primer
gtcagcctcctgggtatttgta

SEQ ID NO: 34 - DNA, 39 bp, Artificial Sequence
  Primer
cacgacgttgtaaaacgacgctttagagcaaccacctaa

SEQ ID NO: 35 - DNA, 21 bp, Artificial Sequence
  Primer
tccttgtgtatcccatgccaa

SEQ ID NO: 36 - DNA, 39 bp, Artificial Sequence
  Primer
cacgacgttgtaaaacgactgcaaaattcaactgtaatg

SEQ ID NO: 37 - DNA, 22 bp, Artificial Sequence
  Primer
ccatacatcaccgaccctcagc

SEQ ID NO: 38 - DNA, 40 bp, Artificial Sequence
  Primer
cacgacgttgtaaaacgacaggtaatggagtaatgtaagt

SEQ ID NO: 39 - DNA, 22 bp, Artificial Sequence
  Primer
ggaaaatttagtggcctgtgtt

SEQ ID NO: 40 - DNA, 22 bp, Artificial Sequence
  Primer
ggttcaaatcccaagatcaagt

SEQ ID NO: 41 - DNA, 22 bp, Artificial Sequence
  Primer
aattcagcaagttaccttatca

SEQ ID NO: 42 - DNA, 22 bp, Artificial Sequence
  Primer
cttagcattatactctcttggt

SEQ ID NO: 43 - DNA, 25 bp, Artificial Sequence
  Primer
gataagggttgaaagaataggcaag

SEQ ID NO: 44 - DNA, 23 bp, Artificial Sequence
  Primer
caaacagcctagagtcactttct

SEQ ID NO: 45 - DNA, 23 bp, Artificial Sequence
  Primer
tgagtctcgaggattgaatgact

SEQ ID NO: 46 - DNA, 23 bp, Artificial Sequence
  Primer
cctagggtgattttgtgtaagct

SEQ ID NO: 47 - DNA, 24 bp, Artificial Sequence
  Primer
taaaagcatgagggaaacagcatc

SEQ ID NO: 48 - DNA, 26 bp, Artificial Sequence
  Primer
taattctttcagtgagggcattatag

SEQ ID NO: 49 - DNA, 20 bp, Artificial Sequence
  Primer
ccccgagctacagagatgga

SEQ ID NO: 50 - DNA, 20 bp, Artificial Sequence
  Primer
agcacagcccctgtgaaaat

SEQ ID NO: 51 - DNA, 20 bp, Artificial Sequence
  Primer
catggcaacccaaaggcaac

SEQ ID NO: 52 - DNA, 20 bp, Artificial Sequence
  Primer
ccccttcacagataccctgc

SEQ ID NO: 53 - DNA, 20 bp, Artificial Sequence
  Primer
tggtctgagcctgaaagtgg

SEQ ID NO: 54 - DNA, 20 bp, Artificial Sequence
  Primer
tgctgctgctgttaaagggt

SEQ ID NO: 55 - DNA, 22 bp, Artificial Sequence
  Primer
tctctcctctttagcttctgcc

SEQ ID NO: 56 - DNA, 20 bp, Artificial Sequence
  Primer
tccctggttggaaatgagcc

SEQ ID NO: 57 - DNA, 20 bp, Artificial Sequence
  Primer
tgcgctttaagacacgtgga

SEQ ID NO: 58 - DNA, 20 bp, Artificial Sequence
  Primer
gcaaagtgcaagcaaggtga

SEQ ID NO: 59 - DNA, 25 bp, Artificial Sequence
  Primer
tccgtcaaattgtttctcatgttga

SEQ ID NO: 60 - DNA, 23 bp, Artificial Sequence
  Primer
tgagtaccttaacagttcagagc

SEQ ID NO: 61 - DNA, 22 bp, Artificial Sequence
  Primer
agtcgactctctgagatggctg

SEQ ID NO: 62 - DNA, 23 bp, Artificial Sequence
  Primer
caaatgcctggtttgttcttgct

SEQ ID NO: 63 - DNA, 21 bp, Artificial Sequence
  Primer
aggaattggggtggtatcacg

SEQ ID NO: 64 - DNA, 25 bp, Artificial Sequence
  Primer
caacatgaaagtgtttgacagagtc

SEQ ID NO: 65 - DNA, 25 bp, Artificial Sequence
  Primer
aacacctaccaggtggttcaattca

SEQ ID NO: 66 - DNA, 26 bp, Artificial Sequence
  Primer
tgccagattaagaaacttccaccact

SEQ ID NO: 67 - DNA, 20 bp, Artificial Sequence
  Primer
agatctggatcagggcaaac

SEQ ID NO: 68 - DNA, 24 bp, Artificial Sequence
  Primer
tactttggctgcatactgcatatc

SEQ ID NO: 69 - DNA, 21 bp, Artificial Sequence
  Primer
accgcagaggtacttgaattg

SEQ ID NO: 70 - DNA, 21 bp, Artificial Sequence
  Primer
gtggaataacaccaccaccag

SEQ ID NO: 71 - DNA, 23 bp, Artificial Sequence
  Primer
catggtggcaactggcagtttga

SEQ ID NO: 72 - DNA, 25 bp, Artificial Sequence
  Primer
cagtagtcaaaatcatttagcaggc

SEQ ID NO: 73 - DNA, 20 bp, Artificial Sequence
  Primer
cagacccaggcacaccagaa

SEQ ID NO: 74 - DNA, 23 bp, Artificial Sequence
  Primer
tgcagagtgctcaccagactcat

SEQ ID NO: 75 - DNA, 24 bp, Artificial Sequence
  Primer
taataacagaaggggcttcaattc

SEQ ID NO: 76 - DNA, 24 bp, Artificial Sequence
  Primer
ctgtcaacatagtcccctaagaag

SEQ ID NO: 77 - DNA, 23 bp, Artificial Sequence
  Primer
gtattcagaggttctgaggacta

SEQ ID NO: 78 - DNA, 23 bp, Artificial Sequence
  Primer
tcaccaggattctcacatggaat

SEQ ID NO: 79 - DNA, 24 bp, Artificial Sequence
  Primer
tcagttctgggaagatggtgtgta

SEQ ID NO: 80 - DNA, 23 bp, Artificial Sequence
  Primer
ctctggctcgtaactgctaaact

SEQ ID NO: 81 - DNA, 39 bp, Artificial Sequence
  Primer
cacgacgttgtaaaacgactggaactgctgttttaatgt

SEQ ID NO: 82 - DNA, 41 bp, Artificial Sequence
  Primer
cacgacgttgtaaaacgacggttcaaatcccaagatcaagt

SEQ ID NO: 83 - DNA, 103401 bp, Canus lupus familiaris
aaagagctgttttgcaagatgatccatcttgcgaatgtttaaagagatgatgcgaggcgaggaagaacagcctggttaggaggatttctgttctaaaaagctctcctacccaactttccctattatttagatcgaggtttcttaacctcagcactgttaacatttggggttggataattttttttttcttttttgtggggcctgtccaatgcattgcaacatgtttagcagcatccctggtcttagccggtagattccagtagcaatccttctccctcaatggtgccaaccaaaaatgtctccagccgtggccaagtgtctcctggaggagggcaaaatatctcctctccccctttagaaccaccctgctgtcgttggcctacctgagttgttcgagaattttatcttatgtttgtttaactcgccttttatagcttcatagaactgtacagggtgcatccacatgcatcacctcaatttgtacaactgccccatgaggccaggtgttattcccattttacagatgaggcaattgaggtcttaaacagccatcacagggtattggcctaggtgggatataactcaggtcttctgcttcaggtctgcttcaggtcttcaagcggctctttattgtatggagacttgcaaatagatgactttcctggcgattaaatcaacatgtaaaggagactggaagctggaaaccttcctgagattgactagttgaaaattaacaacggtaagctattatctgacagagagtaggctgttctagttaaatattctagtttatagtgagctcccataaatcccacatacacttaatggaataccaatttgcagagaattagagtagaataaaacatgcctaatactctgtattgatcctgaattgtctctggtgatttgccatctcctaaaacggactcttatatgtaaattaggcattagctgaggaatgtaaaaggtcaagttactgaaggcatttttctgagtggcattccgttcaagcaagcatttattgagctgcttctgtatattagatgcagtcctgggtggggatgctgagtaggccagtggaccccagcaaactcagggtacttggaagaaattccctgcaaggagagggcaggtcctcggggacatctgggagcaggagaaagaacccaacttaacacagtcaaattcaggaaaactcacggcttcaccattcctgttttaaaagtcttctctcacattttttcatagagaagaattctggtagctttcaacctcagtcaagtggcaggaacttggtcagaggcaaatcttatccctgaaagaggttgggcttcccagctggtaactggcctccggggattgggactaacttccttgactgttgaccgtgaattgggaaggttcacgtacattgttcagttttagtgctgttctctcccacttttgcagttgagaaaactaagtttagcaaaggcagcctggggccaggagcaagagggaccaactgtgaccttctgaaccttgggtccaggcacagaaggaattgtgttttccaggatgcacggaacccaccagctttggggatttgagggaccgggaaggcctggccccctacctccctgctgagccctcagggttcgtggtacgatggaagggaacctgtgctatggaagggaacctgtgcgtgtgactgtaatcccagtgctcagcttccttatgccagacagcagtggttcctggaaagcggggccagtccctcttcatctgttcccaggtctgttgcaggggctggaatgcctaaggcagtcggcacttgccctggccttgacttgcgctggtggaggcagggcagcctgctgatgtttcccaagcttgggctcttcttaccggctctcttagaggcaggccaggatctggtcagtgtcgacgctgctcgggtctcacaggtgtctcctttcatccacctgaagagggatgaaagggaatgctttggtaggcccctggctaaaatctgatctagtcatgggctccaagacctagagatcttgcatctacatagcttctggttgtgtgtccacctgaagctacttgctgcctccggggtaaaggccagcggacagcacagatttcacagcaaattcagacacctgttgagttgtgcgtcaggccctagggttaccgagaggaaagtcagacccttatcttggaaggacaagtggtagaattgggaagacaatgacaggcctgcgcacacgtgctctgtctagttgtgtgtctcacaattactttaaaaggagataaagccagatgccggagaggccagtggaggcggccattcattctgatggaaatcaaggaacaccataaggagtagttggcatttgaattcagccttgaaggtagaaaaagatagtggggggggagggggtgagcagaacatcaggtgctaaggccccgagacacagagaccttgtgtatttcagaagtgatgacttccccaggtcctgcatcctgaccttggcaaatgcaggccctacctcccatagtcagggtgccaacccccaccctgtgtctggaaggggccccagcctcactcaacccaggggtaagggcccattctccttattctcccagaagtgatgcctgtgggttctttgaggcatgagcactttccgcccaggatgcctttggttttcattatgaaacactgttatggcacttactggctgccaggtgctgttctgaatgttttctaaatattaactcattcatttaaaccttggaacaaccctccggctgtgcttcctacctgggtaccggcagcttgctgggtgggtgggggcagagctcatctgaccagtccctctggggtgggggggggggtgctctgatcatggtatggcttctcctggctgcggtttataccagtgtgccgggaaacagcccagccgagtctgagctgggatggagtactggctgccacgactccgtttccatcggggggccagcaggactccaccggctatattttctgaacggaagactaggatctgtagtctgaaaaagaatgtttgtgctgcttcttcatgtaagaggaggcctgggaggtgttgtttggatactgagagagtggaatttttgaaaccttttgatgctttctgtggaaagttaggacagaggatttgacgttgggatagtcctggaaaattcaggtcccatgatttactgtcaggatatacaagcttttagtaaatatttaagcgatgtgtagtttggagtggattttctgtactgagccccttggtgcctcaaaagggaataaatagtactatctagtcccagaatcttgttcattcccaccagccctaccttgccggtagggtgaaaaaaacgaggctcagagagatggagtgaccacccaaagggcttgttactcataagcaggagagccaagatgtgaacccaagtttttttcctgaacaaatcatttaactgtaaaatgaggataataatggaacctatttcctttaaatggtctattttaaaggagttactgaatgaaaacccactatgacagaacttggcacaggaaagggtcccctccctttgtggttgggcctctgaccctccagacaaatggtatgttcagtgttcggtgccaagagtcactgtgggtgagtaatggtccactcctaggccagggcttcaggtcctaggtctcagggctaaagggacattccaaggagtaggatccaggaccagaaggggagccgtgggcagctggatgctgtgtgatggaaggcgctgggctgactttgggagtggagcatgaagacctgacctcccctgccccccatgcttccctaaggtggagtcagcctcagtttagacccagggtacttgtgagcaaagcagcaaccacccccatcagccatcccttgaccctaaccactagcaggtctggaaagggggtgtgacagtctgagcctgcttcacccccgccagccctgtcccctggtctggttatcaatgagttcatacatggttgctgtagaaaattaaagtgtcagggaacagaaaaaattatcagcgatgttcatcctggaggcgacagtcgccaccaccgctccggtggactccacatgccttcccacctgccagcccgttctctgcagtcacgggggtctttcagaaacacctcctattgctctgagaggagaagactggtgtgggcatctcctgcaggcaggcccccgtgtggcatgtatggtttggtccctgccctgccttacctgccacggggctgtgagaacctggaaccttgcctccccaccccccaactcttgctttgggtgttagtccgtgactgcacatgactggggtaaaactgcctgctgtaaacgctcatgcctccggatctctctccttttacagtgttctcgcagttgcaattttgcagttaattagtgtaattatttgtattagtacctgtctccccagcgacacggggtgctctctgaagtcaggatcgatctggctttgttttcaaaaaaggaaaaaaaaaaaaaaagaaaaagaaaacaacctttctctgggacttatttttcttttaactttttattttgacctaattcagacgtacagcaaggttctcagaatagaacaaagaactctcatacccttcacccagattgcccaaatgttaccgttttatcacatttattttattctgctctttatgtatatatatatttttctcttgagtcatttgaaggttatttatagatttttcctttccctcaaaatacttcagtgtatttcctaaaagcaatgacattcttctatataactccagcacaattttcagaaccaagaaatcattattgaaacaatactattatctaatctgtagatctcacttgggtttcttctagctgtctccatagtgtcctttgtagcaaagcacctcctccccccatcctgcatttcataaggtcatcctgtctctttaatctcctttaaactggaactgttactcagcctttctttgccctttatgaccttgacatttttggaaagcacaggccacttattgtatagaatgcccttcaatttgagtctgttttaactccccactctgtccccagtacgactggaacatcagatgcactccgtaaatatttattgaatggaaggaggtggaagggaaaaagcagaggttttcaagccccggcccaccccccccccctcccgccactactttccttgccgaaggatgttgcagtcatacggtctataggtttgcatcctgctatttttatgatacagcttttcccaaaaaaaagagaggcttcatcaggggcgagcgagcctgccgtccctagaagtatacaagcagacgccgggtgacggcgtggcaggggtgctggagaggagaatggggcgtttgaggaggggcgagactcagcctggaggtccctcgagtgctcagcacctttctctatctgggacgcctcctttgcaggcttaggggccccaagaatcaagcgacgagaattcaccacctaaaccagaacagtttgagataaaagtggggctattaataattgcatcccgtgaactagcttcagctggggctgccctaggcaaatctggagttgtgatcaccctacccaaatgtgactttgaggttggagatcagcctggaaagccctctagcactgggaatcccctgcctggataaaaacagaactaacctccttggcaccgcctgtccagcccctgatcgtggcgctagccctcgtggtggtcacacttcccttgccctgcccagttttgctcctgctacaccaaccttgttgtggccctcccctcgcttggaccttcctgcctggggactagggaggtcgtttacatactttgggctggacggaggaggagggactggctctgtgatggaccaacaagccagctgtgtttcccactgataaattctaatggaatttaagagccagggtgtataagcgaggtggggcgggagaccagagcctccctgcctagaccttatcttttttcccctggtgctcacacttccactgtccttcctgcacaaacaccacatccctctcccagccctagttctgagatcagaaccaagctactggcctttggcagaggcagggaagcaggtggcatcagggacccaggcacaagaggaggcgcattggatgaccagtaggtgtagacagggagttggattcagagaaaccaggcaggaaaccagcgtctcgggattccggtaacaaggtcaatacgaggccatttatcaggaagcctagacttgcaatgccctgtccccagacactggggtcagagggggcaggcacgggggtctgtatggtacagctctttaactcgttctctgcctagcaaaagagaacaaaccccgccttcctggattaaaaagtatcataggataaaaatgtgaaggtttggagatactttctgggccatcccagtcatgtggacttcatgaacacaggccagtgcaggatagtatttttataactcgaggcattggtggctttaatggcaaagccagtgagggagtgtcctcgttcttagaatgcgagacactagcacttactgaactttggaaaggttttctagttaactgtgatatgatgtttttaccagttaaagatattctttctcccttgctgaagcacagctatgtaaaagcgcttggtccaaggtcgcttttttataggacagaaggaatcacagtgtcagaactggatgagacctcagaagaaatgtttatagctccctcgttttatagatgggtaaactgaggtcccaggaggtctgtagcatgctccacggcacacaagtagccagttggtgagcagggactagtcccgcatttccaaccccagatcttaggtgtgttttctagatctccgtctaaggaagtttatgaggtggaccgcatatttcagtggttaacagaaattttattaaccacagaaatgaattgtgttggcattaaagcagttatcatttctggctcttttacgtggaagttgctaggctgtttttttttttttctattttcatacgagaaatggtttgtctcttccactgaatttctgtagtatatactggtttcttagattttatcacatcagaatcatgttaaaacatgggctgctgggccccactcccagagtttctgattcagtgggtctgaatcagactcaggtggggcctgaaaacttggatttctaacaggccccaggtgctcccgagaccaaggactgtcctttgaggacctgaggctcagtggtagtgtcagacaagtgctctgtgctttgcagccagcttgtccccacccgccccccagccacctcccctccatcccaccatgtagccagctctctcccctgttactcccacaccagctaccctgaccattccacgctctccaggtctcctacctgttgtcctcaccttgcctctccagacggcccgtgaccctgaaatgcccagattccatcacctttccggccctgtccttcacactctggctcctctcctctttcttcctgttctccagtctgagaaaacgagtgtcctttggctcaggcttgcctcttcacttacagctgaaggctgtcgccccatcacctgggaaagaaagacttccccgagcccagcttcaccattaggtcaccacccagtcactctcttcccttttactgccaggctggctacaggaggagtctgtggcccctgaaaaagatttgctctcacctgtcaacacagacctcctgctcagttctgtgagtgcagattttctatgcctgtgacaaaagatgtaattgttctagattgtggtaaaatgcacctaagtgtacagttcagtggctgatattaggtggcagtcatgttggtgcgctcctgtcacccccccgtgcaccacatgccactcttcatcttgcaaaacaaactccgtacccattaaaacactaacttcccagcccccagcgaccacccttctactttctgtctctgtgtgttagatgattctagttacctcacatacgtggactcacacggtatctgatgttttatgactagtttattttgtttagcatgatgtccccaaggttcatctgtgttacagcatgtgtcagaatttctttccttttcaagggtgagtaccattccgtggaatgcacgtataccatattttgtttatcattcatctgtcactggacacttaaattgcttccaccttttggccattgcaagtaatgctactgtgaacatgggcatacacaaaggcgtccctctggttgggacttgcaacctcaggcagggtgttgaagcatttgtatagcagtcctgactttatctaccccagcgtcccccagctgcctcttgaccagcactgccctcaccatgatggtcagagaccactcactcatcgagaccagtggctggttctcatgtctcagcctgctgggtctctgtgcagcgggctgattgcccctccctcataccacggcttttatgatgtggctccacacccccctacacacacacacacacacacacacacacacacacacaccatggctcatcttgttttcatccttctgcaaatgtttctgggtagactcaccagatgccatcatcagggcctcacaggaccaggtcatctctgggcagttttggtcatatgtgcatcacatatgaacctctccatgagcgtccccaggtccctcctctgcaccttcttccttaaaaacaccctctccataagtgacaggtcttccaggttagaaacccagggacatcctttactcacccccacacctgcactggacagatcctgctcattgcacctcagacattccacctccagcctgcagcctgcccttcctttctacccactcccccaccctccccacccctgcaaaaaagtgggttagtccctgtgcttctcccatttgcctttactctggcttccttccccttctccccagtaggtccctcacacctatacctccttcatattgctgccaaaaaaatgttccctacaaaagcacctaagatcaccctgttccccccatctggccttgattcccccgtctccaaccccaaccccacaagccaagtgctgtaagcccagggcttcccaagctttggtgttaagttgcagattctgcttctaggatgtctggggttgggcctacagtctgcatttgttacaggctcccgggtggtgttgagactgatccacagaccacacgttgagaagccagactctggaatagatctatgtttcacaaataaagttctactaacatattttcaatctataaataatgcaagaatttttttaatgttgtggtatagttttaatgttttcttttttatacagttcgtctttactttctcaatcataaaggctgactcactgttgcagtgattacttgcactttaaagacattttagaagacaaagaaaggcttccattttttaagacttaccaaataagtcaaagtgtgtgaaagtacaggggtctcgaagatgttttcctcttaaagagctgtatactcagggtcagggctccctcttagcttctgcagggcagagactgtctcttaatcagctccatccctagggcccagcccggacctcctcaggatgttagctggaggcgaggaggaatctgggtcttcctccaagaggagctcatcatggaaaactgttggtgtgatgtgcaatgcctaggtgggaaggggagcctgtgcccccggcccttctggaaaaggcgctatccatcctaggaagagggaagggattcctgacagctggccctggttgaggagcacgttggccctcattttatgactcttaataaggttttgcagaagggagccgattgggagagatgtgctggtggttttctgggagctgggcttcagcgtgggaggggagagaggaggccttggagggctgagggcaaggaggcagggagtacctgggagagtctgacaccaggagggcagcaaggaccacgggcaaagaatactgtacatgggggtgaattgtgggatggatgtagccatttttgaccagcctgctccaaaatattgctaccttcctgtcacccagtaagtctaagctctgtagtctgttagcaaagtgtttcatgacctggcatctcccgcttgtgggacctggctcctgggccagtgcacatgggcggcctcagcccaaagcccacattccacagttttctctgcaaggctgcagggcttgttctctcatggtacaggtggtaagcttgggatcctctctggccccctcccccactaactccatggccctagttgagctcctaacgtctcagtttcctcatcttcacactgggaatgacaagtatctccaactcaccaggtccggggaagtgcttctcccagtggcatcatggttttacttaggagctgcaaggttttcacttgctgcctgccatcctgtctcctcagtaagcttgacaaaaggaccatggtgatatttgtccacaggtggagccagaggtcgcagctggagagagctgtgggtgctgccctgggcagggtgggccccgaggtggtgccacggtgggggatagatgggaaggtccttggagctgaagacacaggcgctctgggggttgggagtccgcttgttgtgtgaatgataaaggcaccaaaagaaaagatggcagctggcaacagtaaaaaggaggaagctgaagaccagggggacgttggattgaggtggagagcagggtccctcagactttaacttgcatacaggaggcttcaggtcctgtagctctggggtggggcctgagattctgcattactaacaagccctcaggtgacgctgatgccagctgcctagaaactactcttttgagtggcattggtctggaacgtgactatggtagggaggaggctcctggagaacatatagaaaaagcaaaataagcgcagctgtacccctttctcttcagctccatggagataccttggagtggcaggtgcactcaggtgtctgcagcctggcagctagattggaatcagattgtggtgatggtggaggaggggcaggaggggaccctgagagctctgagatcagcgttgagttttgacttcgttgcttgatagttcatccttcaaggtctagctttgccagaagaatttccatagaagctgccttcgtcgcaggctgagctttggtcacagcctagacaaggcccagcggccaggggcacacagctgttttcagagcactgccacactgaaaggcgattatttgattattcatgcgtttctctctctgaccagcctgtgacagccagagagcagcgagctgagacagcctcttggtcacctgaaccccagcccttggcgtggagtctggccgcaggtggggctcattaaatgcctcaggtattagggacgaaggcagaatgttcaagaaggaagcgtgtgtaaatccctgtgaccgagagagcaggggaagcaggcactgagctgagatggtgggagaacgttggtgtgaaggaaggaagacgactgaaggagtcttcaggaccttaagtaaaacgccaggaatgtcttctgaagatagaatggggcctcacgagggacagctgctggggatttgggcatcagctctgcgagttgggctgcgtgctctctaaaggagccgaaggccaaccaggggatagggatttcagccaggcacccattgtcttggtctgaggtccaggagcaaacttgcttttcttctcccttcaggagaggacaggtgcccagacttaggcagacaaaggataaaaaatttcagcttcatgagcctgggcagaagcagcagattgaatcaacacgtcagggctggagcatattcatagcccttagcatgttgaacctccctacagaagggtcctgcggggtaggggtccattggctccctggacctcagtgccctgcgggtgtgggtccattgactccctggacctccatgccctgcgaggtgggggtccattggctccctggacctcagtgccctgtggggtgtgggtccattggctccctggacctcagtgccctgcgaggtgggggtccattggctccctgggcctcagtgccctgagaggtgggggtctgttagctccctggaccttcagaggctcccatgaattggggtcagcctcattctcctgagttggagatggtgtctcagatgttgagtaactagctcgaagccacagagctgggaagtcgaggagcaggcctggggtctgagagctgggagagttgggtggggcagcccagccttggagcagaatttctgggggaggattggttgcaggtgtggctgacaaggcccaagcgctggctgcggttgggaggggagttgggggcaggccagaggaagaggaagaagcagggtggggtgagtgcagacacaggagaggaggacgggaaagaggaggagtgatggacagaaggagaaggaggtggccagacttgcatactctgggggccaataaaatatcaggtgacagctttttttttttttttttttaggttctatcagtttgattttcttcaaattcattattgcttattataatttaggaaatacagaaaggctgaaaggataggaaaacctagcaattatatttatggccttgagttcctttataattatctttcatagttgtattcactgtagatacactgtaaaaaaaaaaaaaagcctaaaaaaaaaaaaaccttttttacttaatattgtaacattttccattgtgtcactatgccattatgaagtcttcatgggcatcctgtgcctgttttgtgatttacctacctagccctctacagttggaaattagcttttctgattgtaattctcaatgtgaagcgttgttgggaggaaaatcttggctgatgactttcatgtctggctatatcctggctactaataggttcctaggcatgaagtccgtcagaccacctcccagcattgttgggatgatcttattgctgacacaagccttctgaaagtgcaggtgtaagccctgtgcacattagttgtttgttcactcactcctgtgtgcatttcacaagcatttattaagcgcctgtggggaaaggctccattttctctggtcaggtcagcagaggaacagctgccagcatctctggcttgcttgagacgtgtgggagtgtttgatcacatacggcgtccctgccccgcaagcccccgccagcagggacagcccgtgggtccagaagtaaacttgcaggcttcgtggctgcagggtccaggcagtgctaaccatgagtttggagctgtctgccctgcctgctgtaatgcttagcggtctggtgaatactggccatgctgttccccactttaccttctgttcatgcatgccacagacacctactgaatacttctgtgtgcgagacactattccaggggtgaggggtacagcccttaactaaccaaagtccctgacttttaagaaactaccattcccgtaggagggagggatgtgggaattgtgaggagacaagaaataatacatccagtggtgataaatggtagggagaaatagggagttagaatgacaggaagcccatcctgatatgtaaaattcgaacagacacagggagagcgcaagggtggaggatgtctgagggaagcaagcttccagcagtgggaatgagctgggcatgacctgggaacgggcagaagggcagagtgagagcagtgaggtgctctctaggagggcaggagatggaccttgtagcgtaatgagatctaggacccaaggttcagtcttttcactaggaggctttcctgctatttgaagcaattcagactaacctcctacctttttgttgcttatctatggcgggtgatcgccaacgcctgtgttgggtggggaaagttgtggaagctgaaacccagacttgggaaagtcaagacttccgttggcccttgggtctcctcttctgtgcgatgtggaatgtgagctaggaaattggtagctggggctttccagggctaggactccattaagtgccattagttaaatcttggttatttcaagttaacttattagagtgtataaaaaatcgttgacagatgtttctttttttttttttaagatttatttttatttttttaaagagagaccaagtatacgagcatgagcaggggaaggggcagagggagagactcttctggcagactcctcgctgagcttggagcccaacatgtggcttgatcccacaacccatgagatcataacctgagccgaaaccaagaattggacccccaaccaactgagccacccaggtgcccccatagatgtttagttcttagtaagtgtagcctgtcagatttattacaagcaagagttgctcagctagctgtgagtacttgcgtagtgccaggaaggtggtggggatggaacatgccccccagtttatccattcagtcacccatccatcttgcaattatcgagcacctaatgcataccaggcagttggctgcctaccccaaagtggtgatttattaagatttttatttattcatttgactgagtgagagtgcaagcaaggggaacagcagggagagggagaagcaggctccttgctgatcagggagcctgatttggggctccatcccaggaccttgggatcatgacctcagctaagggccaacacttaccaattgagccacccaggtgcccctcacagtggtgatttttaagagacccagatttctgtcttcatgcattttatggtatagtacagcaatgctagccctaccagaggatcatggtggggagggacccaggtcagaaagggcagggcaaccctacctcacaccatatacaaaaattaactcaaaatggatcagtgacctaatatgagccaaagtcctaaaactcctagggataataaataaatcatgaccttgaggttagcagtgggttcttagatataagaaagcacgagcaaaagaaaaatagatgtacttcatcaaaattaaaaacttgtgcgtcaaaggaccttatcaaaaaagaaaaggcaacctacagattaggaggaaatacttgcaaatcctgtatctggtaaaggtatattcagggtatgtaaagaacttcagtaactcaacgtaaattaagataacccaaaatatggacagagaactttgagtagatgggtctctgaagatcatggccaataagcacatgaaaagatgctcgacatcattaggcatcagggaaatgtgaatcaaagccatagtgagacaccacttgacacctactgagatggctataatcaaaaaggataaaaagtacgagagcagatgtagagaaattggaacccccttccattgctgatggggaatgtacaatggtgcaacctccatagaaaagtctggtttcctcaagaagctaaacatagaatcaccatgtcccaacaattccactcctaggtatatacccaaaagaattgaaagggggctcaaataaatgcttgcacggagatgttaaatgcaacactgttcatagtagctacacaggaaacgaagtgtcccacaaatagatgaacaaaatgtgtctctgtacaatggtgtattattattattcagcctgaaaaaggcatggcattttgacacctgctaccacatggatgaaccttgaagatactatgctaagggaaataagccagacacaaaaggacaaatagtgtctaattccacgtatatgaaatatccagagtaggtaaattcatagagacaaagtcgaggttgccaaaggttgagggaggaatgaaatgacgagttattgcttaatgggtacagagtttctgtttggagtgacaaaaaagttttggaaatagtggttggttgcgcaatgcggtgaatgtaattcatgcccatcaattatatgctttaaaatggctaaagtggcaaattttgtgtcctgtttgttttaccacaagaaagacggaggagaagcagtgtgaggtttgggaggagcatgactgcccagagcctctcagtttgattcccttccgatttccctgcacaccggatcagctcaggctccatccttgccacctgtagcctggttctcctttgatatggaagtcagatgtagagtgtggttcatgagggacaccttcccatcccctttaaatgtgtccttcccaagtgcaaaaagattgttctgtctggtggaaaagaccagaaaacagtacatctggaggctttgcccagataatcctcatcgattatctcctcctctgttttgccattttccctggggaggtggggggaggggcctcttcctgtccccttgctgccacatgtctctggtgcttttgccctaaagcagggctcctacggggcccccccttccctgacctttgtggcagcccttggtcacgtgctgcctctcttcacatttactatgagctctttcagaagtcggagccctacaaactagagtcacctggacacctagccatggttatatgatattaccaggtggactattgtaaactgtcaccaaaaaaataccgatgaagtgtttgaaatacctgaacaaatgtttgatatgggataaggagcatgtactactaaaaaaaaaaaaaaagcccccaatcaagacaacaccgtcccttggcttgttctatccaatgtgcacggatctcggagcacaccttgtgtggcagctcgtccgcataactgccgcctccttcccctccagggttagctaccttcgggtgatgggagccaccttgaccttcacagccatgcagctagattctgtgttgcttgcatacctcccacagctcgctaagagcaaggtttagatgctgtgcagctgccctccctggatcctcctggagacccagaccctggcacacctcgaatgccccaccccacagctgtcctcctgagatccttggcttagagctcccgtccctgaaggtgaagggaaagggtccaggtgagctcaagttcactaagaagcccagcctcccgagggctgcagtgaacctgggtttccatcgggactcctccaagttcgaggatccctggaggcaggtgctgtggaagggggcactggctgcgtcgggacggacctgtcactctcagggtctccctggaaggagagcccttggtcacagaggtcccaggccaccctgagggcctggcctcaaaaaatgaagtgacctactcctgttctcctgctgctaataattaaccggggtggctgcctgtggagctggacgggcctgtagatatcatgcacacgcactccagcccacaccttttgcaaaaatgagaggaagtcctttactacggtttcctcaaccctgagagttgtgtgccgcgtcttaactcctgtctaatcagcaagaccttggcgatccaggggtactttgtgctctttcttaaaaaaaatttttttgaaacacctgttaaatcaattgtgtcatcttagaacagagggattagggtaataaatacccaagtcattactcaggtttctgatgttgcagaattagggggaaaaggtcttctggaccatttcctccttaagaggaaatctaccaacatgtgagctatagggagtgtttaccaagattatctcaaaaggtaagaacatacctttagtcagtaaattacagagtgtcgtgcttaaatggggcctcaccatcccccccacccccaccccaccagcagtagtgaatcaccagtaattcaacttccttccgggtcacgtgggccagcagactcactgggcaggtgtgtggtgccttgcactgtgcttgggctcatcaccccgtttggagactgtgacctgagcatggctaatctaccaatctgtaaaatgggaacaggagtattgcccactaggccatggcagagtgaatggagtcaggaggaatggatgcaaaaaaggtatttaggaagctctgaagccctgtaggaccctcggctgttcctcaggacaagaagcctggggatggcaatggtccagggactacaagtgaggtgttggaatgggtgggagcatcagtgctcccagcagctggcggtgcctgccgtgggccaggcaggagggcattgctcctcagaattattcacctatgaggacacagacttaaacagagttcttctgacccaggggcctgtcatcttccgggtggcagggcagagactggagctcaggtctggcttactccttactctgggcccccaggagcccccatggcagtggtgttgcttttgctctttgaaatcaaagaactgactttgtatttgtacagtcccagctcctagcgcgtggaaggccccagtacaaataaatgtaacccagggcagcaaccagatacattatttgcattcagatcttcctaagcactgttactctgattagcattttggcaggcccttataaatcttttttagaatctatgcgatttcctttcattaatcttctgactccatctcaccaaaccctaaagccccggctgatttaggctgcataaaaatacatcttatctgggtttctagagacgcccggttacgtacctaatataacccccgagagatggcccctcgggttaaaaccttacaggcagggggttctaaaagctgggaggagcctttggcgggctccagagctgggaaatccgtgaaaagaaatcagtgaaaactaaagagaaatggagcagcagcttcccgggccgcttggctggttagtgaccagtgggatgatgctttcagggggagggctggagggtatttttctgagtttccttgagataaaatgatggcgagggctgctggtggttgaggattgggtttctctggccaggccacccctgccttcagactccctggcaggaacaaagctggcagcatggtcctggtgtgggagattccagctccttggctgggatgagttgcgattagcacatctcaaacctttcaaagtgactttagctgttttcttttccgcaggcttctgctacttttggctgtaaaggggtgggggctggagctcaggatagaaataccagggactgacacccaaatgggcgtgtctctgccgccagggcaaatattagcacagtatttttgtaaagtttatgcaacttgattcatgtccctgtagagtaaatttattccatcctgttttccagtctgtttggggcctcatggaactatccagagggagaatggaaagagactggtagcttaattttcgatggaagcggctcagtataaaacattctattgtctgtattttgagctttatgcattgcttttttccatgcctattcttttatcccccgcccccttccaacgatgggccattctagtgtttttcaagaacgaggtctcccttcagaattatttcccatgaatagtctgagaaatccagttttaacagcgctgccttgagtgggcttacatgcacagaaacttaaccttggcatcgttaaaacagaagtcctaataggagcatggtcttggaagctcgtggtgaacccatcccacctcccccagcgccagcctgccctctgcagccttctcactgaaggtctgatccagcccctctgcttgatcacctctggtgacgaggagctccctctctcctgacacagtcccttccatagttgcgtggcctttactgttccaaagctcttccttatgttgaagggaaatcctgtttctcttaaccttctacccattgaacccttggggtctaagtaaaccagtctaatgagaatagtaataagtaccttcgattggacccagctaggtgccagttaatgcgcatacatactcagtgcttaagactttggaaagcaaatttctccccttttacaggtaaggaaatgggtgagtgatttctaaattcatgcagctaccaagtggttaagctggattccagcccacatctgggtccattgttgttgacctccccgaagcctaatctcctggaagcgcctaatctccctgagatttctctaggataccagttactccttagacactcccgctaaatctctttctttggagatcataaaatgtgttttcccccctggcttttgcatatgtactgtgttggaacccattctaccaagtagactgttttattctgcggaattcccccacgagaccattgtggactggtcccactggccatcccaggcaggttgggagcccttctcctgttcctctggtgtcctgggaagccatctcccttagacactgaaactccaggcaccttactctccccgcctcgccccttcaaccccgcctgacacagagcaggtgcccagtaaatgaacattgagaaatggtccggaactcctttgccaaagtagtaatcaaggctaaaagtaattataaacaagattaagggtctgtgaaattgtagtcccagtccaactgggactctgtgggactttggccagtcgttgggaagatgtaagaacagcttgtctgggaagtcctggcagtccttttcctcagcatggtttggttcagtctctctaagggctggcaggtgtcccccccttctttcttccgggaggcaagcttgctggcctctgtagtttctgggttcaccgaggtgacatcttaatgggagaagttcactaggcactgttcaaagccttacctgtggggagaccacatcccagccttctcatttctctgtagcccacactacacaggtggcagaggcatccacatgcatgctgaaaacagccctgggagctctgagtcctgggaaatttggggatgggggtgcagtttaacactgtaccatgataacattttttaaacataaacaaaaacaacccgaccttctaaaaaaatctcacttaaaaaaaaaaaaaaatctcatgaaagggcacctgtctgtcttggtcagtagaacatgtagctcttgacctcagtcagggttgtgagtttgaccccattttgggagtaaagattacttttcaaaaattctcgtgaagatagtgttaaaagtcagaagagtggtcctctctgggggaggtcatgttggaaagaaggcatgggggggctcatgccaggcttcttgagagctgtgcactaaggagttgtgcttcccctcccctcccccatgtgatctttgaagggggtggtgataatgacttcatttattagactcacaagtctaacatgaatttttctcattgcttgggggcaaactttgtgttgagttcctcagggtctcattgccatcctgaaaactttgctcaaaactttgctcgtaagaggttggcttctctttataacagcgttccctctttgcccaaactgcatgcagctcccacagagctctttatcttgcccttcagggagcttaaaactacttcttgttatcctcccagtatcaagtataactctctcattttgttagttttgcaagtgtcatggtgttgaacctataaataaaccccgactccataataagaaaactatttggcattttgaagttgtaggagaaggagggtgctaattaccggtctctgtttgaatacaactaatcctacaaattactccaccctgcttcccagggggctccggtttagagatccactccctgaacaaattacaggatctatggaacaaatgatttgatcccaagactgtacttctggagtgtgaggattaataattgcaagataatttgagtattgagctgtgctattgaagtgacttgagtgagcatcttctctcacgtgatgacaaataatgtgacatgtgagagttaagagctttcataaatgtgtttatagaagctttatttttcctctgaccagatttattgaacataagaaaattctatgtataaagtgacctggatgtgctgcttcaaaacatgatcttttcttactactattctgataagtcagtccattgagtgtcaaaaagcagcttactctaaaattaacagaaaagtgcccaatgcacactaaataaatggcttaactactgaaactttagtcattctacaagataattaacataggatggagcccctatgacaggctgctggagaattggtgagacgtcaagaggccactttgtgcactgccactgtgatgtcgtcgtgtttctggatcataatgttcccattatctgtctctagacacaccacaggaatatcagtggggtctgaggtcagcttagctgcttgctgggctagaacagataccaccacagcatgctcatcggacagggtcccattgcagctcagattaagtccttgtgaatctgtgcacaggactccccaacagtggatggattcttcatggtatcctccaagtgctgctccaagtggccttcatcctgctgccttacaccaggcccaggatgcatttattttttaatataaatattacttttatatatgcatgccccaaattaaaaataagtctcgtgcaccaatacttccattgtcagctgtgatcattttcaaaccaagctttaagctttaaaaatgctatttaaaaattaattcatctgtaaaaaaaaaaaaaattcatcctgagtctctatgccaaaaaatgagtgcattggctataatgttgaattagcaatatgaaaatgccacccttcatagacttttttttttctaatttagtgatactcgtagtgattgtttacattataaactagtttttaaaagaagcctgggtatttatcttcgtattgttatgcagtgtcgtactttggatattaaaaaagccaagaagggacaggggtggtgattgccagaaagtagtgttgataaatttatggaaattggtcatgaggaaaattagatttcatgtaccaattgtaattttatcaattgcacataatttgaaattattaaagtgaagggcgcctgggtggctcagaccggttaagcatccaactcttgatttcggctcaggtcatgatctcagggttgtgagatcaaggcccatgtcgggctctgtgcagggtgtgaagatgggtgtggaacctgcttcaacttctccccctccctctgcttgcacacaagcgttctctctctctctctctctttctctttctctcaaaaacaaattaaaagggacgtctgggtagttcagtggttgagcacctgtcttcagcccagggcgtgatcctggagtcccaggatcgagtcccacatggggatccctgaatggagcctgcttctccttctgcctgtgtctctgcctctctctgtctctcatgaataaataaataaaatcttttaaaaaattaataaaatgaaatagtatatatggaagtgttttttttggaagtgtttttttacggtatatgggttttttaaaaataaactaaaatcactgattggttgagctgagtttcgttaaaaatatttgcaaagcctaataaatcacaaaacatgtgcatctcttttccttttgtcactttccttcctcgccacggtagtaattgactttgtcccccatttctgaggaactacattacagaaggcactgctgtcttttttataacttggatattacaaattgtcaagagtcactgtgcaagattgtgaactaatttgagtgaacaagccaagacatctggttacgtaatatcaagtgatagaatcctttttgaactaatattattttcatgttgctttcttcctctgttatgacaacagtgctaatcccagcactgacatatagactggaaagccggtgttatttcaacagcgtcttaggaagggcttccccagactgagtaaggggacagctgctaagctacagaaactcctgttagctctgggaagtgatttgtaatgtaggaaaactgacccagcctgggctgtaccgacctaaccaagccccttggctgtagatagggatgtttattaaaaatgtgacttctttctgacaactaacgcaaggcaaggtcctgatttggatcctggatcaggaggaaagaaatcaaggatgttagaatgatttgcaaaacttgaatatgggctgtgttacataagtgctttatcaatgttaaattggctccgtgcagtaactgtactgtggtttgaaaggccctcgttcttgagagacatgctaaaatattcagaggtaaaggggcatgatcttgacaccttctgagtgttcagtaaaaagcataaggggtggtgtgtgtgcaggagagaaagcgtgttggcaaaatgttaattagatccaggccgataagggtatatgagagttctgtttttctataagttgggggaaaaaattaacttttaagatgcaatttccttggtccttaccaaaccataatttctgtgggtggtgcccaaggatctatttttaacaaattcccccagatgattgaatcaagcacattgaagtttaatttaagtctttggtttaaaaagtctcgttttaatcgctatttctttgttagcttaattactgagcatacatacattctcacagtttgatctgcccttaatctagcacttaataaataaatcccccatctaggcatttataaatctttagaagggaatcccccacttcagaggtgttggaggaactgttaagcctggctgtaaaacagaccagagctagaagccatcatcctatgcgagatgcagtcagccagcagtcgggaggtggtttatgttctcctctgggcatgcagtgaagtcttgagtggtgtgcatttgcccctgaagccttggttaaaccatgaatgcagcctgactgctgtgcatttgcagggaagaggaggagaaggcaatattggtgtttgatgagtcatctctctagcatggctgatgatccttgagtgtttattacttttccacgctctccatcaggcatctgttggggcttagaaatatcctccccacctcccagaagctgttctgagtttaccagggaagaagagttagggaaaaggagagaattgggatacaaagaaggccttctgtatcgtgagggattttttttgaagttgaactgggataagaaggagggagcgtggctgtgcctgttcccctccaggaaggaaacaggaagtcctagggtaggaggtgaagtgttttcctctcgagcccactagatggtagcatcggcctaggtcagcttgatgagtattccggttgattcctgtattgagccacttgtggttcatttgtaaggtttattcacagaccaggtgttaataaatcaggattgctttctttttttctttctttcctttcttaactgaaacatgcactcttcaactagaggcctaacttcttattttgggggggaaaaaagggtatgaaataaccttaacactttcttttaaaatgaccaaaatggaattgagttgctttccgactttgtctaatggagacgagcatagttctctcccttttttttttgtctgtggttcatgagcttgtgaaagaatagagttccccaggtctatgtgtggactcgggaaatcaagcctcaccctcttccccatcccaagctccagggcagacagggagcccagaagggatagtgttgaagccttgttacccacccctacccacgagggctctagcaacacacgtgccccagattgccatactccaggcgacgtgctcctgctcctggaacccgagtttatagagcagctgataggactgtcacacagctattgtatcttttaaccttaaaagctgtctttattcaggttttaagaaacagaacatgggatcactgggtggcgcagcggtttggcgcctgcctttggcccagggcacggtcctggagacccgggatcgaatcccacgtcgggctcccggtgcatggagcttgcttctccctctgcctgtgtctctgcctctctctctctctctctctgtgtgactatcataaataaataaaaattaaaaaaaaaaaagaaacagaacatttgtatatcttagaaggtatttgaaatcagcattgcctacaaaatctggaatctagttgtcacctgatggcattaacttagcatttttaatgagatgccttttatcgaatacctagtaggtgtctcacattggttagacactttacacatgctaaccctaacacccttggtatgcactggggcccaactgtgcaccaagtgagaggtggcctcttcctatatgtctggcaaatacagtctgataaaaactatagtcacagtagaacctttgttgtgccatacagatactgaagccataacagattggtttttgttttgaaaaatgtgaagagggatgcctgggtggctcagcggtttggtgtctgcctttggcccagggcatgatcccggagccccaagatcgagtcccacatcaggctccctgcatggagcctgcttctccctctgcctgtgtctctgccccccgcccctctctgtctctcatgaataaataaataaaatcttaaaaatgtgaagaacaagcttggcttccatattataaaatgggtcagttgcagctgtaatcacttgtggttggggaggccggtgtgttcctggttggggctgtggctttgtttagaatgaagaggcttataaaggaagccatagaaagcctgagttccccgattgctgtggcacaggaaacaggccagtcaggcagcatccattgtcttgacccgattgtttaagatagaatttcaagaccatctgaaaacgccccttggttgttcagcatgaggcttgctctgatcaaggcttgctgcaggaacaaccgtgggaagaacatttctgcgactttcatgaaattcctatttccgaacgttgtcattaggtgtcaagcccttttctattagcagagatcgatgtgtggaggaaaaagtgtctcttctgggttactgatgtcccttgtcagctggctatgtgactgtgctggggggggcggtcattaatcatggtcaagttactaagtcccactggtacccacactggtgccagctacgtggagctgctcaggtgagcagcagtttgaaaatcagtgcccgcgattagcccgtcagtccatggtactgtcacttggaaactcttgttcaacagcacctttggctttttccatccagggtgctggtgtgactagcggttagttttagctgttgtgaggaactgccctcctgttgcacgagtgcaccctcagtgagtggctccgatcccgtgggacctgtgttctgtgaccctccgagacacatcctgaatttccctgaaaacccactcctctggccatcgactgcctggcccttcctttgagggcttcccagaggaaagtcattctgtgccacaccccaggatgctgaatcaccgtccacgggtgtggccaggacaggcctttttttttttttttttttttttggaaagctccccaggtgatacttatgtgtacctcccaccaggaaccgcacttacaatgattctattctattgtatctttaaaaaatatatagcccacctgaaattcttagacaaagattaaatttgacatttttaatgcagtacggttaaggtattatggatcacatttgtagtcttttgatgacttttgccattactcagaaaacgcatccctgcctctgatgacattttcgatggaaagagcaaggtggaggtggtggggtgggcgggattgtgtaagtgagacagtaagttttcttccgcggcccgtcccaaggatttgttttgctggtattagagatttccctgtgcctgactacagaaggatggaggtttgcctggagctccttcaggagcaggaggacaggggaccaagtgcaccttccctcctggaggctctacctcctttgggttttagcttcctgcagctgagctatgggcctgtggttccctgaggcttcgattctagaggcatggctctgctttgtcctcccccctcacttgctgctgattcccagatcttgatctctggccctgcctgacatttagagctgcttttccagcgtggaccctggagccatgtgggaggccaagaaatgcagactctagggcatcactcagatgcacttgaatcctagtttgcacctgatcaaggtccccagagggtgtgttgtgtgccaggagccctggtcttcgggatgtccccaccctcattccatcctcctttcctgctcccaccaacccacgtttgtctgttaggtagcactgtttctccagtgcttccgttttcgtttcatccttcctttgttcagctctttattggctgcctcccagaggtgggtgttgggagatgcagtgagcaaaaccagagctgtttcctacccttgtgaagcttagcttccagtggcagaaggagatcttacatacctcaccagctatgactgtggaattagacaagcatcggagaggagagtcagtaacgcaaatggagagtagttagggacaccagggaagactttgctaaggaagcgatgcctgtgctgggtactggggcttgaagaggaacagagagaactttctaggcagcgggaattgcatgtgcaaaggtccagctcatcatagaaagtttacagagaattaattaaaaaaagaacattaaattgcactcaagtcctccatctttatctgttcttccatccagcctcatcagattttaaaccttctccattccctaagctgtggagctagcccaggcccttggctttcctgcctgcgtgatgcggccctgtgctcagtcgcacacttcctcagtcttatttagtcttccattctacaattgcatttgctgggaaaacctctttcctcactgttccctgagtacaccaaagtcagattgtagactaacttccgcttcatatgtgtgttattttgagtgaatttgatatcaaaatatacagaaaagaacacaaataagaatggagcttgatgcagctttacaaagtaagtacaccggtatcatcaatacccagatcaatagatagggtattataagcttcccagaagatctctctctctctgcttctctctttctctcacacacaccctcacttgatgcggaagtgaccacgcttctgtcttctattagcattgattagttttgcctgtttctgaactttatgtgaatgggttcaggctattggtaatatatcatgtctggctgcttttgccaacataacatgagaagaatccatttgtttctggcctaacaatagttcatttatttcctttgctataaggtcatgcgtggtatgactgtactgtaatttccccatcctattgatgggcatttgagtcttttccagtttggggctattataaaaattctcatgcgtgtcttttaatggataaatctacattctcctgtggggaatgtgcctgggagtggaattatgtcacagggtatgcgtatgttcagctagtagtagtaaatacggccagttatttttccaaaatagttatgctaattttcattcttaccagtcgtgccagaaaggcccagttgctacaagtgctaccacttggttttgtcagattttattgtagccactcttggtgggccatatagtgctattactttgtggttttaatttgcatttctttgacgactaatgtggttcccattttttacagatcgctttgttttttctcttattgatttataggagttctttatatattctttatatatgcatattgcagatatctctgtgggttgtcttttaagtggtgtcttttaatgaatgtaaattatttaatgcagtttataagcgtcttcatgttagatcatttttacaccacagttctgtaaaaaaaaaagtagaatacacaaatatacatttttaaaaaatgataaaagattaaaagcctgggaaatagaaattaaatttagaccggggagataagacacacaaataacccatataattctattaaggtattataattgaacaataaattttgcttagagccacttagaagtcaaaattcaaatggaaacatggtctgttatctggttcacattcattgtgagttcttcagttttatgaggggtcaaagcagacaagggatggatggactgtgaggtgtatccacatgtaacaggcaatttgattgtcagagagctaacaggtgtaccttctgttataggttattcagcgatagttatgacctcccggttacgtgcgttgggtggaagaattaatagtatacgcacctcagaactacccaaagagaaaactcggtccgaaatcgtctgcagcatccgcttcctagatggcttggtacagaccttcaaagctaatgtaagttctccaaattcaatgttttctaaattaatgccactggaaataattgttcttcattttataagccaagagatgaaaactattttaaaatgaaaccctgagggggtgcctgggtggcttagtcggttaagcgccaactcttgatgttggcttaggtcatgatctcatggtcctgggatcgagccccagatcaggctctgcactcagcaaggagtctgcttgcccctctccttttgcccctcctccaggctcgagttttctgtctttctctctgactctcataaataaaatttaaaaaataaaataaaagcttgagatctcagttcttgtatattgttattgaactggaaataaccctaaaaactttgcccacattcctgttcacctggagtgtcttcccccgcccccccccccttggtgacatgaacccagagactctttgatgttacttcttctctgtgacatcttcgtcagcttcctgaattagttcttccataatggtgccctgtatgctttagtggagcccatatcgcatttcattaatagtagttataatcactagattatatgttctttaaaccgtgagctcctcaaggacagaaatagtatcatttccccctgagcctggtccagagccgggcacttaggacatgtgtgagtggatgggtccaatctgaagggtggtgagacagttccactcatagatggctgggctcaggagagcctggctcctcccacctcaagcccacgacagaggcaggaccagaaacgcagcctccagtctcaggagttgcctgagttgcaataagatgcctgaactgtagcagtccatgctgcctcactccaccccagtcactgcttcagccaatgacctctgcctccctctcccatcctctagccaggcctgaaggggtcaaccccaagtctggactggctgccagcatctgccccaacctggaaagttcagtgtgcaaggaaaggggtcagaaagagaagagtgggggaaaaaaaagagtgggtacaggagaacaactaggggtaatagaaggcccaggacttgaaagggtaacagtaactggcccctgggtcttccattcccacctctaactttgcctcccgctggccccaggtcaccatgggccttgatggctgaatagaccagaggagagtgctgcttacaggaggtcgcagcatggtgggtgctctcagaaggtcacaagtgtctcttccacgtgctataaactgacagtataagtctggctcctttgttaaaatgtatgttttctgtctaggagctatttcttcgctatatatcttcctcctgacaagaacatgaatgtttatctaacatgtttactgagcacctactatgtgccaggtttcacgctgggtataatgaatatagcagggaacaaaatggcaaagtatctggccttttaaaggtcacatcctcgtgtgaagaaatggaccataatgaagtgtgtatcaggtggtatgtgcttagaagagaaataaagtgggatgaggagggtagagcatgccagagcaggacaggggtgggattgtcactctacagaaatgatcaggaaagacctcacctgaaggaagtgcaggaaagaaccatgtgattctctggaagggaagcttattcctggtttttgaaaagagcagagcaaaggtcccgaggcaggatcctactcagtgtagttgtagtatggacactagtatgtctggagtggaacgagcaaaggggagagtagcaagagaggtcagaggtaattggtttcaggatggtcagggggactgtgggaagccttatgggtcatcctgagggctctggctttattctgagagaggcagcaagcattggtagttttgggcaaagaccttacataatctgactcatgatctcactctagatccccgtcaaaagtggacttgatgagggtggggtggcaagggctaagagattattctaacagtccaggggggttggggaggaagagggttggtggacacggtggtggtagtgtggaggtggctaagagtaaaccaagtctgaaaatatttttgagggcagagcctgtgggatttgccaacaggtggaatttgggatgcgaaaggggcctgatggtgactcccaaggttgttggcctggacaagcagcaagatggagttgtagttaagagatgggaaaggctgtaggaagggcaggttgggggctctgtcatggacacgtctgaggtgcctgttcctcatccccatagagatggtgatacacaaagaagaaggttccggggagggtctaggctgggatacagatgtggggcttactagggcggagatggtatttcaagctacgaacctggatgaggtcccctgagaagtgaggacaaggaagataggaacatggttcagcgtcggaaggttagagggacgaagaacaggcattacaggggatggggacagactgtcccggaagggaggaggagagcaagagccaatgtctgagaatctgggctaggaacctctttaaggaggagtgatgagccctgtaaacggggatgaggaaatcgcctgcccagagcgatgatcctagaacacagcctggcgctcagcgggcgccctgtctttcttggtcacaggcaccgctgttcgtgtgtgacttccccaagccgccagcagtcgggtcaaagctgactcacgctgaacttgaacctgagggttctggaggtctggtggcagctgaaaccccatttgcgttagattttccacttgcagggatcgttggcaccaactcttgaggcatcattgggaagctggcctggacatccgttcttagcctggcccagggagcaagctggtcacggacagtgacctggcgtagcaaagatgacctcttgtggggtggggaactgggctgagaggcttgttctttataaatgtgtgccttattgtatttcagttccttttgagaacttggaaacttgggcagaaatactagaaggggggtgagttacacactctacaggggagatgcggcgccatggggacgtgcaggctttgacagccaagggcctggagtcaggcaggcagaccatccatcatcagctgatggcatatggcatggagcctcccagcacctctgtttgctcacagccaaggggatgctggtgatgccttcacgagtagctacagagagtagagatggtttaggaaaagtgcacctcgtatcatggcttgttgcgggtgttcagtagttatttaccttttaaatacaaaatcctatagagattttaataaggggaaccaatagctctttgatgcaccaggagagtagctcgtaattcaggaggagagcatctcatgacttgcagtatgtggcctgggctgtctttcccccactgtcacgattccctcgtgaaggaagctgtcctgggtagaggaagcagcaggtgcagggccctcgggcaggaagggggatgtgtttggggaaatgggtgtatctcctgttcccacacttatgaccgaataatacaagatcccaccaaagaagaggttgcaggagggggcctgccagctacctaattctcctcttcctggtcccagtggcctgtttccaggtacatcatctgctctcaagcactcagaagaggagctgtcatttgcttgttctacgtagtctatttctgagtctcattgtccttaagtcaatggcttggttcaaaagattcctgagaagcctgcccgataaaggagaatttgcgatttcccagcggcaccatgctgtctccctcccctgtgcctttgtaccaggccctctgcttaatgttgccttttcttaatttcctctggaaggggacgggtgacagccctgtcacccgtccccctcccgggtcaggagttgcttccgcttgtccccaggttgaattaccatctgtgtctgttctgtgtgtttactgggcccagagtgtctcatggcacttgaccgcatctgacacaccacgtcctggtcactgcttccacgcctggcctctctgggacacttactgtggctctgtctccgtgctcagcatcgtgcctgctccgtaagctccgagtggtgatggtagccgagtcctgctctggacgtgaagcgtggccaaatgtctccactggcttctcacttcagatggcggagagcggagacgcagataatccctcagggtgtcacgtatcaagtcactttgagggggtttaggatggcattttcgtgtccttagccatcttctttcacaccgttagtagagctgtgttccctgtacacactgccgtggctcccaaggagccaacaaactgatgtgggattagcattttgctctgggtaatccacgggtaggtcatggagagtttactctcgtcgtgatctctgttttccaattcagacatcacttgggaatctcagctgaggggaaggaagaagtcttagaaaattaagctatgctatcttaagtcatatttatgtggtttttttagccccacttcccatatataccatgtgacttcttgatgtgacatttattaaagcatttcttactgtgaggtatgagtgcatataaaatctctaaatgttttaactccttttttgcctacagtaaagacttttaaaaagatttattatcttagcacgcaagcgcatgcagggggaagggcagagggagaggaagagaaagaacctcaagcagagtccatgctgagtgtggagcccgacgtggggctcagtcccaggaccctaagattaagacctcagcagaaatcaagagttgacttcctatctgagccacccaggcatccctacaataaagacattaatttgttttaaaaatctgcccactagaggcacctgggtggctcagttggtgaaatgtctgcctttggctcaggtcatgatctcagggtcctggcatcgagtcccctgttgggctccttgctcagcagggagtctgcttctccctctccctctgctcattctctctctctctctttttcttaaataaataaaattttaaagagacgcttgggtggctcagcagttgagcgtctgcctttggctcagggtgtgatcccagtcccgggatcgagtcccatcgggcttccacatcgggatcgagttccacatggagcctgcttctccctctgcctgtgtctctcacaaataaatgaaaatctttgaaaaaaaaaaataataaaatcttttaaaaaaaatttacccgctagacactttttttttttttttcttctagaaacaagacaccggtcaagttcttctggatatggcatataaccacttgggtgtgactgaaaaggagtattttggtttgcagcatggcgatgactcagtcgactctctggtaagtaccagccctttaagacatgctggactcctacatggtttggttttatatcttatgttttcttacccaacagagatggctggaatcaagcaaacccatcaggaagcagttaaaaggtgagtgtgaccttctcatttcagttgcacaccgaacattagtatatcgtggcccctttctctcctccaaccctcagtgtgctcctaacatctgggacctggattctccgtggtccaggtggtatgatatgtctgacttctcagtgagcggagatgcttcccaatcagccctcctcagctatgagcaggaaagggaaaggtgaaagaaggcagccaagagcccaggaacctacttgcttcttataagggaaggaggatggcctctcttagaccagtgtgagagccagggtgggaaggacacatgtgcatgcacacacacacactcacactccctcccactggagcctaagacataggtaggtaatatcatagatacaacatactgctccccagccatgagtgggccccagtgctgtacttgaactcccagttaggaaaggcttcacagcatgccccgtccccaccctccgcccagactgttctcagcctggtttcccagcatcacactctctgttcccctttctgcaccttatgctccagacaaatctggctgccctgccaggctgcacacattcctctgtatcttccttctgtcccttccctcttgtttgaaatgctcctctctctcctatatccaaatcttgctgttcttccaagccactcagactgccatctcttttattagaggccgtgactctgcctaccctcccttacatttgttgttacctgcttacctcccgagagcactctttcatcttgtagttgcctgcctttatcttccttggaaactgcaagctccttcagggaaggatgcttgcaacgttgtctgggcacctcacaatgcgcccccctcctcaggaggcagccagtgaatggctgaggggtagacagcaggtggatttcatacaagcataaacacacgtatgccacctccccccacctgccccagcaccccgatggaaaggagtagagagaaggacccatggccatgatcccactgctgcctccaagcccttacctcccttagcattcagaactacccatggggggccacagagccacggtgcaagtgaatatctgtcccccatttgaattctcagagccttaagtccccacaggcatgaataggtatcatcattcattctctcactaactcattcattcaggaaatgtgccctgagctcagattttggtaacacactgggctaagctctgagaacctggcccaaatcagattgggaccctgcactcgaaaagctgacagccaagcatggggatgtagacaaggagagcgatggttctaggctagttctgagcacatgataggatgttctgggtgcacaaaggtatcctgagacactgtgacaagttacagtggggaggggagctggaagactagtccaaggcagtaggaagactgagctatcccccagatgtacgggtggtcagactataccgagttacagcgccacccactggtacccgagcacctgtgcagaggtacattcgtaggtgaacacaccaccccccatacctttgacacatgccagagagacactgacattaagtactacagagagagaaagtgaggtatcccgctccgtggcaggagcagtgagaggagcagggtggagaaggcttgaatttcagaagcagcgaagcgctgagcacacagggccagcaaacattcagtgtgtaatggagaggggtgccgttgacctgaagaagagcgtggaatcttagcagcacaacctgatcctctcctgtgaccatctccagcaaaagaaatgggctaattttatctctacaagcaaactgactcttggtattgtcgcataaagccatatttgagtatgttaattctataacacttgccggtatgttcatcactttgggaatttactttgcgttaaaagcatgaactgtttcccagtatccaaaagactgaggtgtggcatagcagaatcaaaaacctcagtgctaaagttctgattttgtcctcagttgtgtgctgaaggcccatgtctatgcctgttggcttatctgcaaagcagtgccatcagtatcaccctagcctactcatagtcttggctatgactggtctttatgctcaagaacatacatggaagtgtttgggggaaaagtacaaaatcgctatctatgtgagattatttccttgtgaatgttgtataatctgtcgatttaaagaattcagaaagaaattcctgctacaagtgacggagtgcgttgtttgaacacagccatgtagtagggaggggataatacctcattgtgtgctcgtgttgcaaaatatctgactctttgcagacttggagatttctaaacagtcgattgtcttgtgcactccttcttttgaaattcgtccttctattgatgaaaaaaagctgtggtctagtgaaaaaaaaatgcatcacttgtgggagagtagccgcttacctgcattgacttttttaaagggaaaacttaaatttgtttttgaaaaatgttgtaggaggtttcccctgtactctgcattttcgagtaagattttttatacctgatcccaacacactgcagcaagaacaaaccaggtaataacattttttttaaagtttcctttttaatatatgaaaatgagtaaccactaaaaatgtactctttatcttatcactgtggccgtacactgcatttgggtataaccacaccagcggttatgaaacaagtgaaaatcagaggggtacataattgaaattttcatcagccaattagtttctctgtcttgggcctacttccttccttatttggtgcaatttcaagtacactcccgcccaccacctcctgcccctcctgttaccccaaacccctcctgcctgtggagcctgcaggggcccaaagaaagtgctccttgcttcatttgctaacgcaggcagggtttggaagataacatggtatctctgtggggcgagcatgggggccctttaggatcacaagaggtgtctgaactgagcattctcgaccagatccccagttggtagaaatagaaaggaatgcaggctggaccagctcctctcctgggtgaatcaggctgtgcaggcaaaatgtgaaccagataagggagagagcccagataggattgtttacccctcagggcttggcatagggcctggcccttgttttgtaccggcacgtgggttttctgggtggggaacagtgtttggaatggagggggcgggcacaggaaggtgactgctgtttgaacagtgaccccgcctctacttgccctcccccacagcaatggtgagcccttttattaatgatatgtttatttttcatagggagggcagcttccagagtttttttttccttcaccagaaaaaaaaaaagaaaacccaatgtatgttcaggaatattgactcccattctttgttcactgtgcccatgagactgtgtccaagaaggtgacattaactcaggcatcgagtacggtgttttgttttatttttttgagggtcctcacatgatgacattttagctttcggtttgtttaaaggctaaatattagtctattattctgttttgcctttgtgcacctcttccctccacaatagcagacattggagatttaaccgtgtttactgttgagttaaattgtaagccagagtcagagatgtcggcagggctcattgggtagctgtggggggttcatgtgctttgagttttccccatcatggctccttatgctggtggcctacggggttagtgtaggagctcagttcagaagccaatggacacccaggggtgacactgtctataggaataggcgtttgaggtcaccccactatggttcccccaggccattactaaatatcaagttctgttcaagggatgcattaaaaatattatgacgacacacacagtatatggtgtgtgtgtctacatgtgtgtggttttagccagtggacttttaaatcacagccacgcagcaaagatacattgtgcttcaccctgcagtacagatatgtattatgaagcaagtgtttacattagcaaggacagagcagtctctcgtcttctctatttccttcttttaaaattccaattgtgatccattaaattcacgacgaactgataggtcccaatgccaggtatgaaccaccttgcctaaactagtccttgtcacccctcattctgtctcccccgtgatgctggagccactgggggtgtggcggccagtgggcacgatggaggtctccaaacgtagaactagagtggagggattgctagggctggtccgtctccagtggccctcttggtcattgggcatccacttcccgaccccaccagagagaagcttcctccctctgggtgccaacaaaatcatcctgctgagcctccttgtcatttgagggaccatttgagatggagggaggacttggggagaaaccgggctgtcttgagatctggaagggccaacatgggtaaggggaattagacgtgcttggtggtagatcatggccagtgcggggaggatgcagagaggcagatttgggcacaagatgagtaatagataaatttttgtccttccctgagtccaagatcaaagtttgtttgaatccctgctacagttcactgtgttaggactaacacctgaccctaaccaccccaccttccaacttaagatgcatggtattccccagggtggaatagagtgtagctgtcacctcctgggagtgtgggcaccagatttcaatgtaacaggagccatagcagcagccacccacgtggctcctatagagctggtgggcagccaggactgacagatctttttcttatgctgagctagacttccaccattaattcacgtgtgtgtgtgtgtgtgtgtgtgtgtgtgtgtgtgtttaacttagagttttgccttgttttgcctttattcctgacacacagtatcttattaagccccagatctgtagtttatccagaaataagttccttttagagaaatttagctctcaagtgtcagagaaagcagtatttgttgttttctgtttgttttctagaattctaggcagggaggcattcgttgcttttgtgtgtgtgtgtgtgaagttgaggtcacggatgggatacactctaggaatgatctttacatttctgaagttggatgcgctgagcagctcttctgtatggttttcttttattctaattgctagacatctctttgactgtgagcctccacaactgctgttcctgataaatttaggtcgttgtataccgaatttcatttactggcagtctcgaaatgaggctcctgcaggatttccaagagaccccttactttgggcctcctcccgctataacagtctctttgtatgatgtcatggggttgactcaggcaaaatagcctttgatgaatggtgacatccaggttcatgttctcctttctatccctaggaagatacaccaattaggctcgtcactggagggccagattcaccatcggattatctagcattctctcccttctctactttcttccagtagcattgtaggaggagagaacacttaattctggttgaatgtcagcttggataaacataggaacaaactagaaaggactggtgatgttctcctaggtgaaaaatatagtctccaatacagagtaagtgtgaatcagcaaatgtatgctacacgatgtctcagtgctggtcttcaacacgttaaggatagttgatgataactacaggctatgtgatgatatcagaaacaggaaattgaccctaagctgaagtcattagatgcagaagaaatttgtagcatagtcagggtttttcatagtgtgagcgtccaggttaaaataagatctaactgaccccatgtaacctatctgaatgcctgtttccccaaaaaagaggaggtggtggtgccttcttcagagctggtaggaggctgaaatgaggcagtatattataaatttcccacacccggcctgcatctgtgcagagaggaggtcgtaatgccctgtgaagtagtcacatctcggaacaccagccttgacctttgaatgtatagctataaaggaataaggagaccccatgtgttttaaattgccaaaattatcactctcctgccccagaacactcctgctgcatctgtattatatttcaagaagttccccttttgtcctcttggccagacgctggaatgtctatcccatttgaatctgggctttaaaaaagagtgggagctagtagccaagtgtcacatgtatgccacatcccaaaatataaagctttccatggcccttcgtggctggtgcagcactcttgctgctgttacccagtccctggatggtgggcaggttcacagggcgaggagcccgtgatggcttcctgagcgctgcccacaaatcaggtcctgggttttgaagtgacattgctggtggctcacaagggccaaccaatttcagctcggaggtcctggcagctgatagctgactgatgtttatgtttctgctcaagtacccacagttcctgggacacaggaaaaaactcagctgggtgcttgcacccagccaaggaggtgtccgcatggccccacctcctctctgcagcttcatgcggagggaatgtgggtgtgttctgaaccgcggccccccagcacatgggggctgccaatgcatgatgcctgtgggtcctgtgctcttggaagccccaaatttggttgggaatgtccagggtgggggtgcccactggcaacagcaccagctgatctttggcgggctcaaccatccctgggacagcatgtgctggcgtggggctggctgcaagtcagcagccacatgaaccattgttactgaaggggcagctttgcaacgtacaaaatagtgcctgataattcagatggaaatgtgtgtgtatgcattttgtcctccacatgtacagatcttgtccttatcacttttttttttgtatttccatttgagtacgatgaattaataaaaacaattgtttcgtaggcatttgtatttcttacaactgaagatggatatttgtgaaggaaggtaatgagaatttcacgttcttcttttcctgtcagctgccgggcactaaaattcctggttttttaatctaatgagcgtgtatttctggtaacagtattaaatctaaacaaagggtggatgttgaaagcagcatcctgcacggcattacgctctggacatctggttttaactcagtcttgattttgagaagccgaggatggataaaacttcagttttatgggaaaaggtacagttatctttctggtaaagacctgagaaattttgaagggagaatgtgagacgggcttcattccagaaggcagattggacctaatatagattcacttccaagcccctcagttatctagaggtgattccttttctctctgggagcctttctgctggaagacgagtgtctgcctgcgagtcagcacgtcttgtgtgtcaggaccatctccaggagtgtctgaaatggctcgggaagggggaaaaaatgccaagccacttgtccctgagctccctccttcaaatcctggcttttccttgacctaagggagaacctgcagcaggtgaactgtggaaacagtcactgcttattctcctcattcctgctttcctcctgccagctctcctgaggagggtcttcagaagcctctatgagggagcctgccatgaggttgggggcagtgagacaggagcccgtgaggcgattgggaccagtatggacgttgcagaccccagttgccatgaggatttagaaggagtgcgggctggaggtctttgccctgtgccacccccttggtctcagctgaagagcacatgttgcaggcttacagaatagagttgggaggctcttcctgtcaaagctagacaagcatcttgtgtccagcaggcagaggagagctgttggaggttcttaatcggaaggcatgtggccagagcagcatctacaaagatcatggcagtgcaaggaagggaaggctacaagaggctggaggggcttcatctaggtggaagggagatttgcgggcagggccatgaagatcctggggatggggttgggggaggaacatttagaaacaagacatgggttgctttctggacttccagaacctgttgactgctcagagctcctggaattcgagaattattgtgatttagctccttctcgcactcagagaaggaaggaaccccttctctagcacaactgcaggccttgggcctctgcttaagcatcccccagaaaggagctccccttttttgtgagagagccttctcacatgtgagggtgacacaggtgaaattacccacccctctcctgggtccagcctgccctctgcaccaaaagaaacttgtctctccctctctcctaggcactaatttcttttcccagcaaatgttttgcaagcacctgttacgcgatgagctctgtgctgtgctggtcagataaggccgggcctcccacccccaggagctctctgtccagccagtggaaattggctgtcttcattcttctccaaacacaatacccccgttagtctctagtcacacctgttgaagggcatctcctgccttcaatccctggcatagctggtcaggtctgctggttgccttcaccacctctctcaggagagggtctcctgctaccagacacttgctgggtgtgttagggtgggcctggccatttgtggctgtgggtctttgaacagacgggatggatctagttagccttcttcctgggtgccctgacttgctccaaccatctgtgtcagcggctctggctccgaggcatgctctccttccagctgagaacaggtgaccctgtctgtcagagtgagagtctaaagcctggagggaaagaaaggggtctgcctgggggcatcgttgagtggccatgcaggctctcagcattcctttttagtttgtatttacactattatttggaaatattcttttctgacttcttcatagttgttccttccccgtaaatggaacctccgtagcttctcctcagttccttgaatcccattcatctgctctgcctctagcaggaacactgacttaagtgtctttgaagcccatctgggtcggacagcaagtggctggggtttgctctccccacgggctctctgggtccccagatgaacaaatagttggtgagcatccctggacgtatctccagacatggagagatcattcagcagttctagcaggaaacacctagtcacctgaggaaagttgggggcccctccattgaggaaggccggcttctcgtgcagcttcaggacaggtcccagcccagcagacaggaaggggaaccctgggctgcccaacctgattagtcagagagatgggggtgactcacatcttgggaatttcaggagctgccatggaaacagtacttttcccccacttcccccggggctgctgggaagaatgctcttgaaatctggtttccccacccagcaagccttccttcgtgctgcttctcttcagcctctcctcagcacatttctctacctggcagggatccctggcccgggtctgctggccaagctcagacagatggtggcgttcatggttgaacagagggaagggagcaactgggggactcgaagtatgactaagctagagctggggtcattggcagaaatgggctgtttggaggatagtgagcctgagggccggggagctcaagtataaccccagtccttgggtctctggcaggtggcaggcaaactgggaggcagatggaccattccctacagatcagaaacttcaggagggtaaatagatttaggaaacttaagtagctgttactaagcaatttagaactcacaggagtttttaaattttaagatccttttttatttttcccatctaagagggtgtcccctctctagaaagtgggtaatcaagaatcacggagtcaacaaaagtcaacaattgagttagaaagccctacaaaaagtctcacgtctttactagaaagatatgaaggtggcccattgatctaagagtataattttcctcacttttccctctcagtgatatgagcctggcctctcttgaccagcccaacagtaggcctctagcttcatggtatttgtattaatgtgcattaaatacaacaaagttctcttttaattttgactctttagtgttatatttatctttactatttgaattatatcctaaaattgtctttgttacgatccatgatgtttttttaatggttaatccatgaaagggacaggtgttccccatgatggatggttaaggctgttgttggttcttgtgactttgctaatatctttttctcctgacaggttaacctgcccccttaactcggcagtggttctagcatcctatgcagtacaatgtaagtcacccagggagcatttccgagatggacagactgttctgaccgatgtattaagaaagtccctggcctttcctgtttgaccaggtcacttcccctcttcccaggaatcccgttgggcatgcttcgctcccactttcaccctgacatgctccgctggcacagagatgttggttagggaaggtgtggctgagcccatacagcagaagtcacctatcacacattgccttaaccagacctttgctcatgtattagatcttataacaagccccagggggcagatatttctaacagtcacctcttagaaacatggaaaccggatcccacagttaaggaatttgctggaggccactcatgctacttgcttaggaccgtaggaatttatttttagaatttcagtagaaaaaaaaaaagaatttcagtagagttaatgagttatttattggtttgtaccagcagtgtccagtagaagtataatgtgagccacatctataatgttaagttttcaagtacctagtattaaaaaaggaaaatgaaacaggtgcaattagctttaatactttaattaatccagtatatcctgaatattattttatttttaaaaaatattttatttattcatgagcggcagagagaggcagagacacaagcaagagggagaagcaggctccatgcagggaccccaatgagggactcaatcctaggactctgggatcacgccctgggccgaaggcagctgctcaaccgctgagccacccaggcgtcccctgaatattattttaatatgtaagcaatataaaaaactaacaagatattttaccttttttttttttttttttttttttttacgtgaagtctttgaaaaccagtccagattttatacttaacagcatatctcaattggaccagccgcacattggatagaaagtctgtgttggaatttcccattaagacttgtttaagaattcactcattcagttgaactttgttgagcatatataccacatcaggcccagtggtaggtgctgggtgtgcaccatagaaagggtacatagccagatggagggcaaggtatcacttcagagttagctgtgtggtgccactgcagtacactaacagagttacatggtagcccaagactgaagaaggctcctctacctttctgtaagctccacaccataactcttttggctggctgtatggtagggagttgagaggacaagatggtcaggtctcagagcaagtgctggctaaattactcactctaatttggtctcagcggcaaaatagcaagagaaggctgacaagaactcctgaggtgaccttaagtctgtccacacccctggcttcaaggaacaggaataaaagaggtaaagagcaaagtgggcctatagcttacctgcccatctgtgtgatactgggagatagaagacagacttaacagtgaccctactcactagtcagctaagacctggtttcaatctctgcagatagcctgcatctcctcctcagtgggaaactgagaagaggttgcacagtggtgggtctgggtagatttgaaagttcccctctccctgcagtgcagctggaacagactccgctaatgttacatccgggtgggaaatgacatggccacagtcaacctgtatccttactgatccctagacctcatggattccgtctctgccaggctgatcggctgaagagacaaacttagccacattccagagtgagtttgttgttcacagtgagagcttcaggtttgagagacaggaacacttttagaaaggacatagcttcggatgcattccgactaaatttaaagcctggctcctccatttgccacgtacgtggtattacctccctgaatcacccatttttccatctgtaagatggaggtgaggatgttgatgacaactcagaactgactgactgagtccaatcacacatgtgagatgatctataactggaaactatagttggacccttgccgttaactgagtcgatgagagcacacctgaagccatattacctgggctttggctcttccacatacacacctatgaccctgagcctctccaggcctcagctttttcatctgtcaagtggggctaacagtgtttccctatggggtagtagtgaagatagaatgagtgattatatggaaaaccctgagaactgtgcgtggcacacggcaagtgttaactctccgttttcatgtcttcccagaagaaagctttctaaagaactgcctgttgtaaacaataaactctatttagagtaaaatttaagagccaaataagaaaatcctattctaagtgtcagtggtcgggttcccctctgccaaggatgatagactgagggtaaaggactgtattagcacctagaaagtacaaactagtttcagcagaaatagtagcaagatcaccatgctaataaaatcatggattgtcagacgagcattttttaagtataagctctttattccttcactatcaggaggtacatatgtatttttcctcaaacttccctgcacacatttctgtaagattaattgtgggatgggatactgctggatagtctacatttttatctgctccctttcactaaatagtcccaacagggtgtgtcgtagcatgataagccccaccagtgatgagcaaaccaaaagcacattttcttattagataaaaagcaatgtaggtaaatactgaaaagactgtaggtggctggtgacctcaattatttacgaggctcagagaataaagttttcagaaatacataatttatcctgtaatgtctgctgacctctgtcacgcgcaggctctgcacccacctcagggcacaaagacagacaaagcatggcctctctctgcgaggggtgtcctgcctggtgggcaggcaaaggtgggcccgctgatgctggcgttgagggcgagcatagagaatacactgcatcctgcgtggggtgagaagcgggtgcgtccaaaaaagcttcttagtgatgaccactgagaaaaacctccaagatcaagttggagatggacaaataacatggaaaaggcttataagatgcaaaggactttcccagcagaagagtgtaacctctggaaatgctgcacatttgtgtgcctagaatatacctctctctttcctgaacaatataaaatattagccagaagaatgcatggaacttaagacaatgtatttcccaaataaacactcatttccttttcttcctgctataattagctttcgcatgcgttgcttctccacaaataggaaagtgtattaccctggaggttagggtatgtggtagtcagtagatgaaggattttggactgtggtcagattctaggctccaagttaccagctgagcaaaatgggcaggtcccctccttgagtctcccttaccttaactgtttcatgaagataaaagcaatgcctgtcactgggttgtaatgagcatctaatgggttgacggctttgaaaacatcgtgggaagtccctgtgctttcctggtgtttgcagatgactgtttgactttgtttccctctagtggttagcataggactttgcacagagtagccaactaagatttcaaaggaagaccaccctactgggaggctcattgaccatttatttaagttcacatatttgtcagtgacttaacgaaagacagccttgctattcatatttgtagaagggcctgggggttgggacttgttagctaatatggatgaccaaatcaagatccaagaggatctccatgagctataaaacctacagagtaaaaatttttttaaagattttttaaaaatttagggatccctgggtggcacagcggtttagcgcctgcctttggcccagggcgcgatcctggagacccgggatcgaatcccacatcgggcttccggtgcatggagcctgcttctccctctgcctatgtctctgcctctctctctctctctctgtgtgactatcataaataaataaattttaaaaaaaataaaaaataaaaaaataaagatttttaaaaaatttattatttatgcgtgagagacaggcagagggagaagcaggctccatgcagagagcccgacatgggactcgatcccgggactccaggatcctaccccaggccgaaggtggcactaaaccgctgtaccaccggggctgccctacagagtaaaatttaataggactctgccttgaggctcaggacaatcccttgtagcatttgcctgccttctggggacatgaccgctgtcctcagcaaatggcccaccagcagcagccttggacctggcaggctggctggtaggttaggagcaccagctggggcatgcatcacctgggttctggtcttgattgctgaaagtggcttctgtgaccaggaagtcccttctctggtccagtttcttcccttttttaaaaaaaatttgattgcttcttcccttttaaaatgaatttacacaaggtaactttatggctttctcaagctttcctccctgggtgctctgtgtcagggcatctaaattatttcccagatgtactgttgtacatttcaccattgtacagactcacctgagcgtcacactggtgcttgtataatccagcctcgatgtttctgtccatagcttagctgcatttggctggccttgacggccaatggtctgcccttgaccaaatctgcatgcgtagatcacttgactttctgccgccttccccttccaaaatcgaactttgtagtagcttttatctgggttagttccaagcacggcatccctgagcacctaccgtgcgtgtactctgcctaggtgctaggccccacagatgagtctgcctcactcccagcccccagggaagactgtaaggaggaagcctccctttacgacagtatggatctcaaagagcagctcttatgtgtgatggcagttgaggatgagtagggccacacggatgtaaagaaagacatgcttgcatctggcttctcatctttgtgatgaaatctcccgaagcagctccctggctcctctccttcccttctgagattggccacaaagcatcactgcagcttctgccaatccccgtccttgtgacacagatggagctatttgatttagaaatctgtgttgaatgggtactgtgaaacatcagtcctcagaagctcgctggcgtggagaccagctgcaggaagaattcattgcaacctctggaaagatcaaggttaatatttttaagaaaaagatgaaagtgtcttattaagaaaatgaaataccacataattgcccatcaaaactagaacttattctcctatttttataaagctggaagaactgaatcgtggagaagggaagcaggtgtccttgggtcccacaagccagatccttggctgttccacccagggtctttttcccagtctcaacagtccagacacccagacggtttgaatcctcagggcaaccatgtatgcttgtacaggttggctactgcacaactctcaggaatacctatggccatagatatcaatgaggcatgtttgtagaatggcagtcaaggatctcaagggagaggtactgtttggaccagagatgcatctggaaatccctctagaaagccactttcctcgtaaccttgcagttgcaggtcctcgtttccctgattcctctaagaatgtttccttcttcccaagaactctgccctcagccttgccagggcccagacccatgagccccacccactcccaccccctcatgccatcccccaccttccctctgggcctccgaacacatcctggttccatttcttccaaccttatccaaattccaaggagccaatcaaggtgcaagtaataggtcagtgtgacctctttgatgcccacacccattgttttagagcaggttggtccagtgctctaatcacctgggggtactagactgcctgtctgcacggtgcctggcatacaggacgtggcatttccgagtgggcactctcatgtgtctgtatccggggtacagtgcaaactgctgtgggtttagaacaggacctaggtcagactgcttaaacatctggcctcagggaaagagcattcagctgaagcatgcatggaggtacttgacccatgtagggtccgataaccatgtggaatctgaagccaacgcgaaggccaagggcatgtggtggtagaacctctcggaagaatgctccccatgctttgcttgtgatgctgtcgatgcccgtgaatgcagaccgtattgaaaggcgatttcttcctcattctgtcttggtagctcattttggagactataattcttccattcatcatccgggctatctttctgatagtcagtttataccagaccagaacgatgactttttaacgaaggtggaatcgctccatgagcagcacaggtgagaggaggggtggggtggtggcctactttgggaacccaggctgggcatttcggaccttgcgtccttatgtttgatgtttgcttcctttagttcacgaatcctcttttaagctgtcgggggtttgtgaatctttgagctgttatgatggcagatatatttggcataactagcatttctgtaaagtgcaaaatccagtaagagtttagggggatttacagtgttcttttttaggatgtcactgaaatccgttaattcctaggggaaatgccctttttaaagcggaccgccccacgaatgacctaccaggcagacctttgaacggttggccacatttaataaaatgggtggctcctttgagcatcgtggaatagccaggcatctggactaaggtcgccaggctgtgtgaatcccacgtcctctctgggatcgggtcacaaaactccctaaattcctgaaaggagccccagtgtcccgcagctgcctcaggccaagcctgccctccaccagacaggccctctcccggcctcctcatcctttgcctcgttttagtccaagctgcagtggcatctgttcagtcccaggtcaaaaaagcgccatcccaaccgggaaggccaggtctcaagggacctgccattctctggaaggctgaaaaaacaaaggccaagcagccagcgtcccttctaaaaggcaactgccaatcctccctcccagaattgtttcccaggcagaagccagcatcctaaaatagcccagtgacgtctgggagctggaggctgccggaagaatagataaaggtggagagacctgaacttggaacaaggaaggtgggaatttcaaaaggtggcagtcttctggatctgatactgagagcccactgggtaagaagtagtatttagtaaaggatggagcaggctagatgaagtcctattcggtggacaagaaaaggtcctggtttagtaggatccagggttaagttgtttaagatttataacataaggtatctaatgaccttacaacaatgttatgaatttggagaggaattttatttttaaagttggaattatatactctattgattggcaaactctgcgtcttctctcctcttctgtagtgggctaaagcaatcagaagccgaatcttgcttcatcaacatagcacggaccctcgacttctatggagtggagctgcacggcggtagggtaggtacccttggcccagtcgggttcagaaattaacttctgaaagttttaaaacttcttgaaactgccttttccagaataataataataataataataataataataataataataaataataaaagaccttcctaaacaatatttagaaactttataaagtaatgttgagtttggttggttatttatccagttgcttgtttcatgtttccatggggtaggggcgtcatggggattagaaacaagtagtttttgtaagatgatttcatgttacggttaaagaagcgaaggaagtatatgctcattagagcgatgctttaatgagccaagggtttaactgtttcatctccgtgctgctcaaaatacagctaactgctcaaatatgaccgcttctaaaaattattgaaaactattttaaattgattgttccttttaaataaaattggctaatatctttcaatgactgagcaatttctctctgccagacacagtggcttgcttatttcagtaatgccatgaggtgagtatcctcattaccccattttacagatggagaaactgatactcagggacttctcaagggtcacatagcacacaagtagcagcttcagtacgtgaccccagcactaattggcatccaatctttatattttctaccagtaattgtcccacatcatgtccaaaaggaagcaaatattggttgaacaactaataggtactagccattgttccagatgcttttcacaagaccacaaggtctcatttaaagattattagcaatcttgtgaaatcggtatgctaatcatcccattttacactgagaaaatggaggctcagggaagttgagtaaatgaccagctttacatctgtagagtatttttgtaattcatagacacactttcatttcttttgtgcctcttcggttttataaccctgtgataggcaagttatgacctaagtaataaaataggaaaaagatatgagatcttagaaaatactaactttgggacttgatggttaattttcactacatctgcaaataataatttgttgtagaatgttgtcgagaactttctacactggtgaagtgggctttatatgtgtctgagttgatctgtactctccccaccacttgcgtcccatgagagacctcaggccagatgattacctgcctggctgacaccccccaaggtgtcagagggagttgggttaccttcggctatgctggatcacccgaaaccccctaagtatcaataactcaataccataaagttcattcttcagtagttcaaaagctgcgatgggtcccaacaaccctccagagtagccttccttcatgcagtgacttgcttggcagccaggctgcttccgtcttgtggcttcacaagcttcacacatggcctcatgcttgtggcaaggaaggaggaagctggaaggcggggtagcagctcctaaaatgattcagcccagaagagacccatcacttctcctagaacattgaccgtgcctggtcatgtggccctgctgagaaatgtggggatgggcatgggaatttagtgaacagtaaccgtccggggatgagatgcatgtgatttcagtgggttgcccaatagtaaatgctctccggacttttgcatgtgtcagcactgttttctttggacagtttctctaattccccacgttcacatcactgttcacaagtgatttatccctctatgtctcgagaggcaaaagagaatgaaagttcattacacctgccccatattctctaatttcagcctgataaaaacccttggggaggtgaatttgataatcgttcttgtacaaaaggaaaaattgaggctcaatagcccaaaataacacagctagtaagtggcaaacctaccattcacactcagtggtaactaagtcagactgtgcccccctccttttttttttttttttttttttttaaatttttatttatttatgatagtcacagagagagagagagagagagagaggcagagggagaagcaggctccatgcaccgggagcccgacgtgggattcgatcccgggtctccaggatcgcgccctgggccaaaggcaggcgccaaaccgctgcgccacccagggatccccctcctccttttttaatataattacaagtatctgattgggtaaattgggatatagatgactcaggggaaggagcccttttatttttaaattttattgtactcaccttgttcttaaatgactttaaaacaccctgtagttagtcaccatatcttctgtattttcattgtcaatggcaatgcgtggcacacaggctcttaatgaatgtggagtgtactggatttataggcaatatttatttattcgttggatttaaagacagcagtcctccccttcaaaacaatagataactaacgcctttttagtgatgggagctgagattagtgggaagctgtgcagagggtgaggcctttctgataccagcatgatatctaattgttgaaaatgtataatcggatgctcatttgaagattctgtggaatgatgggtggacctgcctggtaacagagtctctaagctgctgggacccaggtgtaggtgccaggcagtgtgcattcgctttagaagcatctggtgctgagtatttggatctgaagtatcccttgcctgttttccataacgttttccctttggagcttatacatactgctatttataaaagcgtgggtgcttgagttaccagagggcatcggcatgactgtgacacgctgtgaatctgagttcttcccttttacaggatctgcacaatttagagctaatgattgggattgcttctgcgggcatcgccgtgtaccgaaaatatatttgcacaagtttctatccttggtaagtgcaaaccagttctaattatttttctgaatcatgctttaaaaatatgccgaacaaaagaaacgttatgtctaattttaaacttttatttctgaaggaggggtagagactttaccctaggtttttacctctgacctctgggaggcttgacccagattcagtacttgggaccagtagttgggttttcaccggcatccttttcccccagtcaccctattgttgggatactctttagaaacatggataaactggtacaagccctgggcagcacattggccagaattcagagaacttcctctgggcaggaattgtattgtcagtgtggttcctcagttagtagttggccctttaccaagtgttcagacttgaatcctcccaagaccctctgaagaaggcagtgctctccatggacttctgatgtgggactccacgcattcctagttgcatgatcttgagcatttaggtgaggtctctgagcctcagttttgctttccgaaaatgggcataatcatggatttcaggactgttgcaaggcttaattgagggactgctagtaaagcttttagtaggggacttgtgtataaaaatgctgtctgtgggagctctaatcgtgtccttatgcaaataatagaggagctgttgacacgcagccacaaagtaccagagctgggatgtagatccagctctgtctgtcctcctgcttcctgtcctcctgctgctgctccacgctgccctcagccgttctccccagacccacgcaagctcaggccaagctgcaacatcacaaaagcagctgaccacaaagagagcacattgcccatggttcttgttctttaaaagttacgtcataatccaacggcccttatgtgatcctcagcttcagtcagctccttggtatctggccgaggcaacgagctgccatcatttctaccctgaggtaaattctcggaagctaggagcccgccttcctcatggtcacagagtagaggacacacatggagaggtgtcagctgccacccatctgatccctatgtgccttttgtgtgttttatttttttgcagggtgaacattctaaaaatttctttcaaaaggaaaaagttctttatacatcagcgacaaaaacaggtgagttatgtctgtgcttgctttgtttcttgaggttttagccacctcagccagtagctctacggcaggtgaaggcagctggccatctgttgggggagaaagagcttggcgtgtggtggacggaaaatgggagtcggaatccagagttcccatagaattccctgtgcttctctggttggagatgtaacttgcttgggagtcagtgagatgggtttggatcatcgctcttccacctccactgaccagccaggcaggtcccttaacctcccttagcccaagctgtcttgttccaggaatgcagtaggacctgcctctcagggaatcaagtacattaaacgaaagagcgttggtcagagcacctggcatgagcccacacagagtagcaatgaatgtcaacaagatgagttccttcccttgatgtgtttaacgtgcacaggaggggcacctgggtggctcagttggctaagcgtctgactttggctcaggtcattatcttggggtcctcggatcgagccctgtgtcgggctctgtgctcagcggggagtctgctcctccgtctccctctacccctcctcctgcttgtgtgtatgctcactctcagtctctcaaataaataaaatctttaaaaaaataaaatgcacagtcatcttcgcatatgtctcctaagaggtgaccacccttctccagatgactctggatagagaagaagactcaaagttttcgaaactgaggctcccacagttttctgaatttgaatcagagacacctgctctttccacgataccacactctgtcctgttatactctatatttttttacttctcgtcgctatgttcattttccagggtttcagagataatatgtacttaaaatgtctccatatagcagtgaatgcttggaaatgaggacactaatgctcaaactagaactagaatctgggtctcctctgacccccaccccattgcaagattgaaagtggccccaaaatgcagaaggtggtaatttaatttgaaaacaaaagaaaagcttaggtagttgaagatcaaggaaatcattaaaatgaaaatatgtgagcacttgatttaattccgtgctcgggggacaaatagagaaatccacaggcttataaaatatcaagttgcagagaaatggaagagtgtgtttcttcaggagagattttcccagagctaaattttaaaagatgtatcacacagcctttttctggttgctccaaatatatccaacctaggaatgtaagcacattcaaaatcagagtcttcatgaaggatcacaagtgatggtaaagagtagagtcaggtcatctttaccacattaataagatggttaacagatcctgttcctgaatattcctgtaacattgatttatggcctcggacattttgaagctggttttctgctctctgttgcaccttacctggtgtagacacagaacacctgtgagcctctgctccttcacctgtggagatgaaatctctactctgtttttctcccccagactgaatccagggaacatatcgtggccttcaacatgttaaattaccgatcttgcaaaaacttgtggaaatcctgtgtcgagcatcatacattctttcaggcaaagaagctactacctcaagaaaagaatgtcctgtctcagtactggactatgggctctaggaacctgaaaaagtgagtattcttaagtatccacagtaggctgagatgtaaatatgtcctggtcactgccgggtagtagacatcctcgggggaaggggttgctatcatccagggaccccagacactggagctggagctgcacggctgaaagtgtctccagggacagcagggtctccttccacgaggactcaggtactgctcgtgttctcatccttcctttcggcctccacaactctgttgcccgggcagcgcccaggcagccagaattacaactgctcccactccatgtccctgtaagggaagcacgtggcagtgactctgggggttgtcgtggtactaagacaggctctcccacagaatgttaattcgatacagtgctcccgaatagtgaatgagcagagatgaagtttcccagagtcaagtgacgctgggacataatacatataacagtcacaaaataacagcaataacgaaaactggcaattgtttcctttgtaccatgcacatacttaattatatttaaatcttataacaatcccatgtgtactagtgtctacaagagtgataatcagaatatttcatgagcagtgcagcgaaagaaattactcttataaaattttacctttagattaaagtggatttcctaaactaatccagtttgtatttttgtgtatttattctaagactgccgcctttctttttttttttttaagattttatttattcctgagagacaaagagaggcagagacgtaggcagaggcagaaacgggctccctgcagggagcccaatgtgggactcgatcccaggactctgggatcagggcctgagccaaaggcagacgctcaaccactgagccacccaggtgccccctaagactgcctttctaagtaaagatacaacagaaattatcaagttaattgtaaactgtttgttgacttgaaaaattgttttcataattttgtgttgttgatgtggctcttactaatgaaacaatgccccctgtctgaaccattatctcctacgtggcactgtatcagactattccagtgactttcccattatgttgataatcattaggtagctttttatagactcaattagtatttctgttagcatcagtgatagtatttatggtgctaagtgctctctctcaacttctgatgaaccaagaacaaatatattttcaaagtttctagcttctataatatcatctagtattttatacattcagtagcccttcctgaacatgtcacagacgtaatttattggaatatagtattcactttagttggatctgagccttcaggccagctgacaggattgaattgggagcagttgaaaacatggattttgctaagaaaactgagttcaaatcctactacctgcatcacttttagtttgaatttgtttgattttttctttttttaactgactgagccacccaggcaggcacgattttttctttttttaaaaaagattttatttatttatttatttatttatttatttatttatttatttatttatttatttatttatttatgtatttattcatgagagatacagagagatagagacatagagggagaagcagaccccatgcagggagtctgataatgggactcgatcctgggactccaggattatacctgggctgaaggcagatgcccaaccaccgagccacccaggtgtcccgaatttcctcatttctgtaacatacagccaacacagagttaggggtgaggtgggcaggttctgtttatccattatagcaaaataaactacccctattttcgtatgatagaacaagcctcttggggctcctgggtggctcagttggttaggcgtctgcctcgggtcatgatctcagtgtcatgggattgtgtcccgcatggggccccacgcactcagcgagtttccttgtcctctccctctgctcctcccctgcgcacacacacgctctctttctctctctcaaataaataacatcttttaaaaacaaaaaaacaaccatctttctgttctatcttgagattttgtgaattgagaaattgagaagggcttggccagccacctcttttgctccatgtagcatagaccagggtttctcagcgacaagcagctggtggctgggctgggctgggctgggctgtaggtccaagataacatggctcccatgcttgatggaatgattggaaggctggattcgcttggaccctccacttctctctgctctcagggcctcttcatgtgcactctccagcagggtcatttgatgtcttgtgcagaaattcaggctccaagggcctaaggcagaagcaggcagtcatcatcaaggccaggcccagaactggcacagctactcttgatcagagcagtcacaggccagccaacattcaagagtccaagaacttgagctatttttaatctgccgttgggggtcatttgggatatcggtggttgggggccatggatcagcagttctttactattaagaccaacttcattgtattgcaaatttcagccctcacagtgtccatttcatagatgagaaaattgaggcttagtttacatgatttgcccaaccctagagctacacacacagatttgcatatggggcctcctgatagaggttccacataaaaggaacttgatcagaattttgcaaacttaagagacattgggactcctccccgccccaatcaaagagaatctattaataatctgtaacacatgggcagcccgggtggctcagtgtttcagcgccacctttggcccaggacatgatcctagagacccgggtttgggtcccacacctagctccctgcatggagcctgcttatccctctgcctgtgtctctgcctctctctgtgtgtctcatgaataaataaaatattttaaaaaaataataatccatgacacatactatggggaactatgaccttaaacttgtgagtcctctcacgtgctccccttgttctgaggcagtgttctcaatcaatgctaatttgctttctgatgtgattctctcagtgatgctgcagtgagcattttgagcagggtgacaacggagatttgttactgtaaattagcacattgatattctagggggacttttgtgttcttctggggtcttaagacattgtttaaaatttaggaatgtcagaactgagtttcttttaaactgacttcaaatgccatcctttcaagtagttcattccttgaggggtctcattagataagatttaaattctgtaggtgtcaataaaacatgaaagtcagcctcagggtgtcccgttcatgtagcacgtggcatggaactctgctgccgaagcatgttctcgggccttctttcaaagagtgctgtgatttagggctcatggcttctgccagctaggatggcaaagagcaccttcctggaaggatggttcatgtttgtattcaggccattgtgagcgaagcaaacattccttgacattatgcgaactttatgcgtgttatcctgaaaattcctcctcatctcgtggaaaggtttggttatatccttaaagcagctttattgcttgcagtagtaaggcccaagaaaatattttcaccggaagagaagaaacaacaagaaaaagccaggacagaaaggaaccacttgtgatctgtgtacctctactccttgtaaccttggctggtgtcatccaggtgctaaccagcccttcatcggaaagggagcatagatagactgtttattatatgctgtaatttcttgttgaacacgtctgtccatgtagaaagtgaaccttgcatctggtttttcaggtctgtaaataaccagtattgtaaaaaggtgattggagggatggtgtggaacccagccatgcggagatcattatcagtggagcacttagaaaccaagagcctgccttctcgatcccctcccatcactcccaactggtacgtcctccttttttttttttttttttttttgtattacccaccactaaacactttatgtctcttaaaaattcagctttgtaagttttcccaatatagatgtggcctgtagtcggagtttatgtttgcatgcttggctgttacagaactaacagttggctgtttttgtgaaacatgcaagcctagctgaacattttccttagtgtgtaatgctgggatgtcctttttaaaaaacttgcctggtctgaatttgagaactacagcctactgaaaaaaacacccctcttggaattaaggggctcactggcagattaacattctgggaagccctgtgagtaggaagcccgctgagatttatttaagctgtttttccattaacatcacacaaaatgaactaggggagaacgtggtaaaggtacacggtgtaggatgcgtggccctcagcaacagtagtagcaaatgcacgtggggaagtagcctgccagcatttaacatgatggcttaacaggaaggaagttgcacttggcgaagtgcgaaggacgtgtgtggctgcatcacccagactcagattttggcttttctgttaattcacaagtttctttacctctctggtctcagtattcccatctgtcagagggttcaaacaatcaccacaaatgacagtgtacaagtgctttgtaagtgattttttttttaaatgaggtggaattattatcattattttattagaatggttcttcagagacattggatcagttttgtgaacgtgtccaaaatggccaggcactgtcagcaggagggaaactaagccagaaacattgccccagctccacgcaggactagagactgtggctttgtattcatttcacagaagacccaggtggacctgtaaattgccaaggagggatctaggaggactgggggagattctctttcttttttctctaacttaaaacccttttaatttgtataataatttatgtaaatggaaatggacagtatgtcttagaaatcggaagaattctaaaagaatccatggaattcagtaaatttgataaaagcagtaagaatgtttgaacattcatcattctttagggcactgaatagtaatgcctgagaaagtctaaggaaaattatacaaaatgagcagacacaaaaggggttctgaaaagaacaattattcgttattattgcctcaataggtataatcagtgatgatgataatagtgtttttgagtacttattactaactcatttcatcctcacagcaattgtatgaggatatactattattatttccatttaaccgatgagtaaattgaggcacagagaaataatataagtaatctaaggttagatctgggatttgagtttagtatcactgggtcagagttcatattcttttatttttttttaattcaattgattaacatatagtatattactagtttcagaggtagaggtcagtaattcatcagtcttatacaacccccagtgctcatatcccgtgccctgcttaatgtaggaccaagggagatcctaacaggcacaaatggagctactgtgcaagacggctgggtgaggaccctgccctggggccagatgctctaggcagagatgctgatggtatttagtgtcagaaatcttaacatgacaacgtagatttgtgtcctggtttcctaagatttaggtacttcgttctaaaacggtaagtttattctgtaaagtagcagcttaattggtctgaaagcctccaaggcagcgatgccagggccccagagcagaggcccaggacagcatccaggagaacagcgggctttgatgtcgccacacccgtggcctggagctcctactactatttataggtcccctcctgaggagtcacaagcctttctcccctactctggacacagtattattgattatctgggccccaagggaataggaatagtggaggccctgacggtgtccaggagaaatggctaaggaagaaggaggtggaaacacagagagaaaatagaaggtggctcagcagcctggggtcacccactacaggagctgatgtccggggaacatcgcttcatttggttcactctcaggagcaggtaggtacagctgaggcccagagaggtggtgtgacttgcctgaagccacacagctgctgctgctatcaggggaatgggatggtgctgccgtgcccatctcatagggcatagaggatatatttttaaaagcaaacctttcttcttggattaagtgaaattcacatgaagggatataattgtagtaaggtttcggttttgccaaaaactaacctttggcttctaaaccctttagttaatactggtgtttgaatgaatctggagaacagatcagtagtcagtacagcctattattttgtgcattaagagcttgagtcctcccagaagaataacccaccctaaatcaacactccccacttcccaaagggcataaattagtctcgtctgcctatttatatggctgaggcttcctttctggaggctacactgcggggctgagagtatcgaggattcgtgtggcccttggtggattgcagtgtactaacgggttaaccggttcttcatttccaacagcgtctgccaaggcatccttagaaaggtcagggcacaaagttagatcccgatgcctgataaagtttttcagtggaatttgctttcaacacttcccactcgaccttttccccacacaggcgaagtcctcggctccggcacgaaatccgaaagccacggcactcttctgcagataaccttgcgaacgagatgacctacattaccgaaaccgaagatgtgttttacacgtacaagggctccctgtccccaaaagacagcgaatccgaggtttctcagaaccgaagcccgcaccgagagtaagaaacctgttctcttctttgtatctgcttgtggtggctgttgaggaatttgcccaggcccagagagagccgtttaagtcaagagctggagcctgcagccagctccctaggctgctctgtgccaggacggcagcataaatcaggctgtcccttctgccacaggaggacaagccgtgtgcctggggtctcgagctcacacttcttttttgtatgacaaagacctaccgtccacgtacgtgttagatttcttaaataagtttccagttgatgatcaaagcccagtttaaatcagttcacattccagaagtatagttatgttaacctgaatagtaatatttctctagattgtcagagttttacccctgcaagatttctggattgctggaaatatgcttatgtggacccatccacagaaagccttctcagatccacagtaggcactcagggtgttgggtggggagagagggttgtttttaatgaattgttagtgtaaagcaaaaacgtgcgtagaacgtctcctgaatttctgttaagttttttgtcagcagttcacgttaaattcttgtatgtccccgctttgcttttttaaatgtgacaaactcccttcacatcttaaggccaggaaaaactgtctctccaaaagtactggcttgggttcttttattattcagtaagctgtatcgaattcacgaagtttcctttttgtcttgattgccagggaactgagagctccaagttaagggtcagttgcatggacttcccttagtacaggtttctggtgtagtccctactcttctttgtcttattcatgggtctttttttttttttttttttctcttaatgaattcacaattcttctgtatccatacattttagagtaagctatcctaaatcaaatttttgaagtggacaagccctaatcataaataaaatgacattttaagctgaagtatgagaggactggatcaaccattagggagtcagccagaggcactgggtattgattctgttagggcccaaactatacctgatatatagaaacctaacacctcccttcacagcgtgggattagggagagggcacttcaattaatctgtcaatttttgccactctagctgctaggcttaaagcattaaatgcatcctgattatattctagacatcgcaggtgaattgcaaatcttcccacttatagtgtgaggttgttacctctctgttgtctctgaccgcagcaaacatggtttccagcttaactgtgttcttaacacctctgggtaaataatcctctggtcactctttccttgtgacctgagctcagatgatgcttttgttgaaatgatcactatctcttgctttaacttgcagccccattgaagtgacagtgtggaagggcgtgagttcctttgtgtgacaccatggttttgcagatctagatctaccgggcaggaaaggcaggcaggcaggcatgacctgagccccttggagtcggaggtggggtcctgtggctttggggaaaggagaaggagggtttaggaaatggactgggggttctgggaagctcggtcacactgtccctgccagccctctctcttgtggcttgcagtcccatcatggaactgtggaccatctagacttccacagaccagtgcagtagccagtaggtaccagtgggtgttcagatttaagttacttaacagttatatcccgttcctcagtcatataaacctcctttcaggggctcaagaacaccacatgtaggtagtgactaccatactggccagcacagagagagtctctttccatcgtcacagaaagtgctgcttgcagtacttggtgctgggtagagagagatggattcttcctggcggggcgtgaacgttgtcaagggttccgtgcattgctgagtgcagcagcacacctcatacacgctatgctgctcacagagggaacgtcaaggcaaattagacaagtctttgccttaaggggcctccaggccagccgagcaaatattggcacattgtcagcctctatcgttgcattgtctgccacttaggtacaagcaagaagtttggggaacggtctgagggttctgagaagcccaattacgtgtcccacgtcagattttcttgctgtgatctgcactgtggagcagccagaccttcacagtccaacacagaagccaccagccatgtagggctatttaaattcatattcaggagctcctgggtggctcatttggttacgctgccaacccttgatggcagcccaagtctccatctcagggtcgtgagtccacacccccgagttgggctccgtgctgagcgtggagcctacttacaaaaaacaaaaaaaaaagaagaaaaaagaaaaaaaaagaaaagaaaagaatttacattcattgaagttactttgaaaattcacttccttaaatcctgtttgattcaagtaatctcaggccccgtgcacccctacgcattgcacatcagttctttctgaaactcctccatgagaccttataagatgcgtcatcctggggccctttttattttgcagctaaatgttaaaagtagcaaataaaggtgattgattcagaagcagccttcgttgcggcagcttcgccaggtgcaccagtagacgcctcacggggtcaggttagcagccatgggttgcaagtagggtgagcagttgaaaatggcagcccttaccacctcggggggcagggggctaccccttcctgctggcaggaagctgcggcctgctgggtcctcaggcagctgccacccccttcccctgagagtctgggggccctcccctgcccaaggcaagcaggaaaggctggtctgtgcccggtgtgtgtggggggaagccaggtagttacctgagtatttatttgcaaggcatttaagaaacgtctggcatgttttatgcagcccggttgtgcaaagaggcgaaaagaagcattctgtcagagaagtcagatgaataataccagacagtccggagggttccttgtagaaaggagaattaagtgtaacgttctgtacccatgtaaacaagaagaaagagaaaggaagaaaaagagcagatagactttggctctgaacccagagcggcgcggctcgggctctctgaacattatcttagcgggtgagtcaccctccggctcgttccgtatttatagcagcacagcaaagtttatgattatagttatgaatccttggcagtttgctttgtgcccgagtcagctgcactcttgttactcccagggctcgctcagcccgtccggggcccttggcttgcttcagagcgaaggccgaactctacagaaaaggaagtttccatcatggcaaaaaataaaataaaataaaaattcaatcccatcactccccaccacccagaaaacgtggcgggggaggaggaggttccctaaagggaaagcgactttgcacagcagagattggaggaaaccccacttgggcgcagaggagggctgtattccaagaactttgcggtgggcgcttcttcacacttgtcccgacgggtcctccgagggagcggggcttcgtgtcgcaatgccccgatgtttgcttgcccctttctttagagcctgccttgttccagaggacacggggcagctgtcactttttatctcaaaactgtgcacgcaacggtgtgaatgcacttaatgctactgaactgtgcactgaagaaaggttaggatggtacattttatactgtgtgcatttaaccactgttaaaagcctctaaaaagaacttccatgccttgagagggactttgggaatgccctcggcagccccggcggctgtgcaggaacgccctgggccctgtcacctgtcacccaggcgccccgctggcaacggggagctctcctgcggtgtggcagcctcttctgggcctcgactgcccgatttttccatcgcaggaagtgttgtccttgacgctgacccagcacgagcactgccatccgcagatccctgcctggagcttctagaacaacacaagtagcgtccgtttctaattctccctggcgaccccgtctgctctcccttaaatgaattcctgctccatcagccactgtccgtcctccagcctgctagtcactgttctctgagtgcacctctgacccgtaccagggggtgacacccggatttggacacggtgatgggacccgtgtacataagggtctctgtcggaggaggccagccagcgacagggctgagccattctgtgtccatgatccctgcacctcgcctgtcctccaccaggcatctgtcatttattttgcagctgaatgttacaagcagcaaataatggtgattcaaaggcagccttccctgtggcggcttcaccaggtgcaccagtaggcgtctttcacagggtcaggttggcggtcacaggttgcaagtggggtgaacaattgaaaaatagcggcccctaccacgttgatctgggggaccccttcccgctgggacgtatgactgatacttttttttttagattctatttattcatgagagacctagagagaaaggccagagacacaggcagagggagaagcaggctctgtgcagggagcccgatgctggacttgattaagggactccaggatcacaccctgggccaaaggcaggtgctaaaccgctgagccacccagggatcccatatgactgatactttaaaccccagggcccccgtctccacctgtcatctctctgtccttacctgtggctcctcctgaccttggcgaggggggccacgttttgctaccaccctgtttgtgcccatatcaggagcttcagtcgtcctcgactcccttttctttgcccttcctcagctaggctctcaccttggtaatcttcctcccagacctgccttgaatcggacccctgccccatgccaccacactgcccggggtcagccaccatcatctatcacaggattgtcgcgacagtgccccattagtgtcctcctgtcttgggtttctcctccttcacgagctgacgggcttctctttgtgaaatctgtgcccccgcacatcaccgcccccccctccccgccccatgctttttcagagtttcatgcctctgcacgtgctgctgttttgtcttttttgttcttagtaatctgcctcaaccaccttctccttccctcttccctatacctcttaagactcctgctgatgtgtctttcaaaggccgggtccagtgcccccctcctctgtgaaggctctcctggctccctgctcttccccagacgtctgagcgtctcagttacagcatgaggattttattcgattgagctctttgagggcaaggatagtgccctgttcacctctccttcccccagacctggcatgatgcctggcatttggtatatattcagtgagtatttgctatggttggacagacagatggacagatggtcacagggcaaactcctgcgtcttcacagtgggacgccccgtctgccgcctccatgctaggtacccatagcctaggaacagcatcacccgggatttatatgccacgaggagagcaaacagatctactttgatgctgtcatcggtgagcagagccatgcggtgggtgctgctgcagggcacatggcaggaaacttatctgacaactaaagcatgaattgacagaagtgtaacaagtgctctcttgctgttgagatgtcttaatgtctgaggggcagcctgaggtgcgggatgaatgcataccccatgattccgttagtgaggaaatggagagcccagcaagcaacccatgtgcaccataccccctgagccacttcagcaactttgtgcattttgatcttgggctcttggcatttatcacatgaagaatgtctccaagtcataacccacacttgaaaagagtattctgtttgccattcaatgaccctgatgtgaagataagaatccacatgaaaaatttctccaggaatcccattcaacacaaacatggcattgggagtgagccagtgtcccttcttctagtccctcgtactcctgttctggccacagcgccaggacatcagcatctcctaagtgctccctggaaatgtgaacctcagggggcatctgggtggctcagcggatgagcctctgccttcagctcagggtatgatcccaggtctggggattgggtcccgcatcaggctccccgcagggagcctgcctccctgcctcttcttctgcccctgcccctctctctgcgtctctcatgcataaataaataaaatctttaaagaaaaagaaagaaaagaaatgtgaacctcaggcccaccacaggcctgctgaatcagagtctgcattttaacaagagcctcaggggacctataggaatgtgccactctcagaagctctgagctcgcaggagcggggaactttctagcccacctttccaccctgcttccaggggaccgagtgagttctgtgctcttctagagtaactcggccaagcccagatgcttcagcaccttcatgcactcagcacctggcttgagcacttgccgcgtgcctggctgtcatatgtactaggagaaaaagagaaagaggatgaaggtctgttccaacctgaagaagctcataggctcgagagagaaatgtgacaccaatgtgacaccaatggcccctgtcccatcacatgacacagctcacgcctcctccccgggcacccagttagagccggacaccctccgtggagcactgcttgagggtagaggtcatcgtcctttcgtaggctttagggaagtgtagagaagcatcacgcaggaggcttcctttcgtggacagtcctgccatttataacttctttggctttgcttctcttctgagaaatgacttttggcatggcttctcttcctgatacctgttgatggcacttgcagttctgaagcatctgcttcctcagggtgactaccccctggtctgcccatcggtctgtccttgcccctttctccccagggagctggaggtggtcagtccccccctggctgctctggctcccagacctcagtgacagtccctcctgagtcccctgagcactctgaagagtgctcccttcctgcctcggggacagaccacttccccgatactggctttgtcagcccacactgtcttcttgctccggctcaccctactcctttccctagaacccaggtacccccggcagctcccctcgtccctcatccccccctcactagcacctcacactctgccccaaggaaggaggaagcccacttggtttggtctcttcactcctggcatccatccagctgccacgttcttgttgcttctcgtttgatgtcctcctcaccatctgtctgtccagcctccacgctgagcagaacgttactgcagcctacagggaacgtctgcacatagctgatcagccaagaaaaaaaatcgtcatatgtttaataaattgctctccaaaagtaacctctctatccctactcagaaaataggattgggcgcataatggctttgaacatactatttttagcactgtgggctgcgttagatagacctgttacattttatactggaaaggaaaaatacttcaaaactttatgtgatgtaagccatacatccaaaatgtaggaattaaatataatgactataaaattagaattttactgaaatcagtctgttctcttctgctttgtcaccctgctcttctgcttccttgttgatacgcccttcacccgttctgtgactatcagcctccagagtcttccaaacgtttgctaatagtgtgtttgctattcgcaacttcatgttaaaggttcagctccacccctagaactggaaatagtgtttcctaaaccgggtttggcctggtgttggtctagcgactgtcaaagcagttgcccctcttgccaccagggggtgctcttagcaacattaaaacggtggagttaccctacctgggggtaattttttaagttttttttaaagctttacgtaaattaaagatttgttccttttcactctgaaaacttaccactgggagatagctttacttaacaaatatcctgtctctgaaatgccttcccatccgttgcaccccttccctcgcccagcaactggaaagccataaggccaaactcttttctagcatgttgcagtgtaatttccacactggtctgtcactgaatttttatatccacataaattgagattgtgatttatcgtgtgaatatgatacagattttatatttagagtgagataatttgaacttgctaggaaaccgagaagccccaatttaatgagaaatccatggagaagggttggagataaaaatttaatgaggcagcagcaaccgtcttttgcctattatgtgtcactgaatgccttgtctactgacctcagtcatctcctgagtccatatgaggttagaatgtggcagagtcggtagaaggtaaggaaaaccctataggctgtaggcattttaaggcatcttctgctaggcttgtggttctggaagggagatagtgtaggtgtgtgtgatgcagagaagtcgagtgtcctacacagtgacagctgagatggttaatcccgtagccctctgtggtggagaggagggggtccctaggaacagacttggcgtggcctggaagagagcgatgcttggggaccccagtccatcagattctgtatgagaaatgaggggaaagctgagtgcctttgggtgagtcactgcctctctagaccacagtttcttcatctgtgaaatgggagttggaccgaggacctcagaggtgtgttcagttctgatagtctgtaatcctcgaagacagcaccgtctcatagaacttcccatgctgatgaaaatttttctataaatgatgtgtccaaaacaggagccgcaagccgtctgtggctcttgagcatttttgaaatgagactgatacaaccaaggaacttcctgatcttcatttaaatttaaatggccacatatgactaggggctaccatatcggaccccgtgtgggctctgagacacgggaggacataaatgagagggcgagaaagtataacctcattgtagacaattcatggaggtcacagcaagtaatgcaaactaggattaatgtaaatcttggatgtggaacattagtcctatttgggcgtggggggggggcagccaagagtcctactgaaacagagctaacagcaggaatgaaatggaaggtaaaagctttcagaagggatggaggcatgcatctgagtgagcttcagtaattaagccaccaaagtagctggcacatttccctgtcaccttcagagcagctgggaacggagctgagttttcagcagctcctggatcctccctgctctcctgttccttttccccgaaaggtgatgatatttggccagacaagggaaagggtggccagtgggcccccacgtgtgccagagccctcggcttgacctttggagctcttctatggttgcagggattcatgacagccctgggatgccaggccccaaaagtcagtaatgcttgctctgctaaaatgtcaatccagttattaagatatctcctccttccccataatgaaggagtatatccgagaacaatccagcacaaagctgcctgacccagaagtcatccagttctgtgtctccatcttcaaatgctccaggctcctgctcaccagagggcgtcgatcagcagttcttagaggacttccacagggtgaccaaagggggctccaccgaggactccagccagtactactgtgacaaggtgggtgagctcgggtcctgtggtacctgagcggccaggcgaccactcaggctttcctgtgccgaggtgcctcatgggcaacttacgtggttaggaccaagagaaaatacctcctcaccacccgttggagagagaagtaacataattaatcatttttgcaatgttttccagaatgataatggtgacggctacctagttttgatccgcatcacaccagatgaagatggaaaatttggatttaatctgaaggtaagttgtgtgtctttttaagggtggcttttaaatttccattaggagtgtgttgcgtgcctaagtctacagtaggaaactggctcagccctcagaactggggaggttttaaagtggccgtaatgctgcctcacaccaagggcctactctgtgtttggtacttgctgtgtacttctttttttttttttttcttaaagatgttatttatttatttgagagagagaggaggcagcagccagcagcagaggcagagggagaagcatactccccgctgagcagggagcccaatgtggggctcggtcccaggaccctgggatcatgaccagagccaaaggcagacgcttaaccaactgagccacccaggcgccccatgctgtgtgcttctttatgtcgccttactgagtgcttataatcggtgaaggtggtatttttaaacccgttttagaaatatgtactagggctcagagatgacacacagttggccagaagaggggtttttgagggaacctgttggcatcactaaagccgatgagagagagaaacagctcaagttgcagggccatttccagggccttcacctacatctgcctgctttggcccacaagcctcatcctatctgtttctgaaaataaagttttattggaacgtggccacacccattcatttacaaatggtctgtggctctcttggcctcgtggtggcagaggtcagtagttgcaccagagccctcgctcccttacctaaaatgttgggtaccctcaggccctttacaaagttagagcagaatgagggcaggtggtctccgctgccaccacggagtccctgtgctgtacccagccccgtgctgggaacctgccctcaggcatgtgacgacttcctacagcttctctctccaatgatgctggaaactcccttcccagcagtacttctaggctgtgcttttagagtcctctaagcacgtttgcatttttaaaaagaacgttgacccctccgagctcatctttagttcatggaatcgtaggggtggcagtgagaggcttctggttatcccactagaccccaaggagtcaggaaacttgtaattctgaaaggattccctgggcttagaatagggtaaagtggaatttgaaccctccatgtgtcagcagtatctgcatgtctttgttgctatgagctattcaagtgtttctgttgtttttttttttttaatttaacatgtttatagggaggagtggatcaaaagatgcctcttgtggtatcaaggataaacccagagtcacctgtaagtgaacatatttcgttctcgcctacgtttgttttctattaaatctaacgttcctatttctgtgtcctgtttctctgccagcaacaaagggggtgtgtaaatgcatgcatatagatggaaagaaagtgtggaacccatgtttttccagaactttgctagtcagggtagcacctgcatcacctgggaactcattagaaatgcggaatctcaggccccaccctagacctcccaaaccagagtctgcatttttaacaagattcctgagtgattggtgtgcactttaacattcagaagtactctcaagaaaaggaaactgaggcgcagagtgtgttgctctgctcactctgtgcacgtgcacccaccgtcccaccagcagcaccctgccctctgcgctccgtgtaggcctcctcagaggatttgcttttctggagcagatgagcagccaagagtcactcactgcgtctcctaaggaggaactagttcgggggttttactcgtaagtggtccttcagcacgtagtttgctttctcctactgcttatggccccacaggtcaggagggcttccattcctacactgttcagtgcactcttctctccgtgccggcttctgcctttggggttatttatgctgacagaggctttctccaccgcaagatagacaaacgctcacgttcggctgacaagccattttctagcagcacatcgagaaaacaaaagatcagaagcctcgcggtgacgacattacagtaataggtgaaggttggctctcgtagagaaatggcttcagtacgttgcgtagctttattccgaaatgccgcatgacttaggtcgtgagcagcacttctttagactcccacaaccagcgaccacgtccgcgtagcagcggatgtaaaaataatggcccggccgcccccgagggagaccagaacccgcacctggtctcaggtgtctgggctccgagagcgcccgcgtcctgcccttgaggattgtgttatggtaaccgaagaaactcgctcttcctctccacccaggcggacacctgcattcctaagctgaacgaaggggatcaaatcgtcttaatcaatggccgggacatctcagagcacacccatgaccaggtggtgatgttcatcaaagccagccgggagtcccacacgcgggagctggccctggtgatccggaggaaaggtaacagccaccgcaggggaggagccacggggcctcagggaagcagggccctctctggcccattccgtggttttgaaaatgttgagctcgtagccagcactggaaaatggagacctttcgcctgacaaccaggatgtgggacttctcacttgaaaaatgtgaagatgcgcccacgcggggcccagcccctcagggccagtgctctggaggtaggcagcacctacccaccccctctagccaggacacgagccctgattcctcccaggcccactccccccacattacgcccctcccttcgccgtctggccctgcacatgtttgctttttcagccccttcgtaaagtgttgggttctgttgacgtcggtaaagcagaagtggggcaggagcctggctgtccccggggtgatcctgcctccacctctgttgggttgagggatctcgagcaagtttttacccaagtatgtgcgtcggttccctccgatgtagaataagggcacgataaccactctgtcacagggttctagcaaagatggaatgatgcttatacttggaaagcacctagagtgcttcctgatacctggtgggcatttgaaggatggccgctgttaaatacaattatttgtgttgttgttgttgttgtaaaacaagccgagtccttggatccttctgcccaggttaaaaatactgaaggtaaatgtgtgtgtaactcgaggccctgccacatctctgttctggttgcacaggtgtacgtacagcccccggtccccctcatacctgctttccctctgcttccataaatctccaagcccagagctgtgctgcatgtgtgaggcctcaccagatctctccagatcccttcccgtgccccccaaaccttgatgccacagcctcccacctgacaatggccttgaggtagaggtcgcctgggctctgtgggaccatgttgttaatagaaggagagacaggtgggtggaagagtaaccaggtcagaacaaaagcaaaaaaagtgaggtctctttaaaaacattaaaatatacatatatatatatatatatatgtatatttaatttattcatgagagaaacagagagggagagacacagagggagaagcaggctccctgcagggagcccgatgcaggacttgatcccaggaccccaggatcacaccctgagccaaaggcagacgctcaaccactgagccacccagtcaccccactttaaaagcattctttccaggcacctgggtggttcagttggttgagcccacttctccctttgcccctcccctgcttgtgttctctcttgctctcgctctcaaaaaaaaaaaaaaaaaaaaaaactttccctttctcttacctttgtcaaaaggctctaagtacaaaacaccatcatttaagactaaccagaaagattagtatgtcattttgtataattagcacttactgaatcattcaagaagtagtgtctttggtgaaagtgattaccatgagcttaatcgtttaataagctgttttaaaccatctgcattcaggtgtcgtaagtataattctttttgaggaaaccgatcaaccagaacgtttctacccctgccagacacactctttcttgttcactacagctgtccactcattcactgacatcaaatccgacgatgagctgaaccagcttttcccagaggccattttccccatttgcccagagggtggggataccttggagggatccatggaccagctaaagaagggccttgaaagcgggacggtgctgatccagtttgaggtaagaggccacaagggccatgtcctgggcctgtggggaagccacagcagcccaatgtctccgtgggctgctcggatgctgctgagtgacccaagtctccaggcagcctgtgctcgtcatgggaacacagcaggggactcagagcacagggcagcagagcaggcccagaacaccgggaacgggaccacagcctggtgctgaactcagcctcaccctcttacctagtaatgctccgtagccatggagaaacgtgtcagaaattgaggaggagggactctgagcgtatttagctgccaacacaaaagctcagaatgctcggcagtggaaggtgctggagaggcagtgtgggtgagccccggagggtggcggaggagccagccagtccctgaccccaccagacaccctctgtagtttggagcaggtaggtagcatcatcccataagggcggagcagccggcctgcccgggtcccccagaaccgtgtgaacggaagtagaatccagaggcagaccctgtcttccttctgcaggaggaacctctagtcatagaattgagcctgtgacacagctcagttcagggccctcccagaacaaagtgggaacacaggagattcggtttttaagtccacggatggattgctagcagaatgaaaatggagtgtgtgtatcttctaaattttttaaaggtagaagaagagtagttcatgccataaaggataacagaagaggataacctaacatagaccattaaaagaaaatagatcgtgattagcgactacttacagagtcaggattcataaattaggaaagagtccagattcggttcaagcgcagatgtcagcaatgccatttgtatttggaaagctacagaaagggaaaagtcacaaaagcaaaactgagaagtttgtcaaatgcatgtggtcccatatggattcaggaaagcataagtgacctcaatattgaagagcaaacaccataaagcaaagactcttaagtgaaagaaaggtttataatggcttaaggagtagcatgtagataatgtgcgttactagagcaatgtgtatccaaacaatagtagaaagtataaaaaaaaaaaactagaaataaaagatgagaattaaccccaccccgagaaaaacaaaaaacaaaaaataacctcctatcttgtggaaatgaaatcagggtaagagggtaggagtactaccattacttggtagaagtagtagacaggcactgagtatctacaaagaggagcagcccctctttccagtgtcctgcaggaggctacactgattgaagattttgaaggacaaaaacgtagaggttatgttgtcgggccctgcactgcaaaaaagttcaaaattgaaatatacattgtaaacactgtaaaactcaactttttaaacactctccaaaaatcattgaaacgagaagataaaagctggttgcagaatatgaataaatacttggcattggaggggatggttcggaaaggcggggagacactttggaggggctcattcagcgagcctgtgttaggtattcaccacatgccaaacattgggtcaggaacacaggggaagagaggatttctggccccaaggagctaatggtctaaaattggtgtatctgggggatgtatcacgagtccctgaaacagctacagaccaaggatagggcaagatttccttacgtaggacgtatgttccctaggaaatggaggtggggcgggggtggttccaggaggtcctccggggtgagatgtattaagaggatggatgggattaagtgtggagggaaggtcatgccaggagggtcacagcctgaaggcagactaagttcctaaggtgtcaggtagtacagggaaacacacgtacctacctgggttgagcagtgccacaaggagtcctgggaggagtctccaggtgttgggagtccttgaatggcgcacctctgtcctcgatcggattgtccacaaggaggcactgtgggtcctcaaagagggaaaaagagcttaaccgtggtcttccacagtgctccgggaaaccccctccatgcggccagctccgctttggttttacatatgggaatcccacgtaagattttccttgagcactgttcaaccgaccttcactgagaggttcaaaaatacttgagagacaagtacccgccatcccctgcagttttgagtagtgtctctccaagggtgacccactgacccaccgcctgcatggaatatacccccaggtgcttgtgaaatggggctcagcccacagctgctggatgagccgcacgcatggggatggggcctggctgttgatcctcctcacaagattggcgggagatttctgatggacacgctgccccagggggaccacagagtggggactgtaccactgctgcccatgtcaagtgtcctctttagagcttacagttggacagcagcatgagaaatggagaccttagaaattttagggttacattttaggatgtgcacgtttcgttcagtctatacgtttcctagaaatcaagtgggggaaaaaccctctttttaactatggtcttttgcaaaataggtaacaccagagctgctccctgatggagctggtggggtacctggagccctgccttcttgatctccccacactaccccccccccaaccaaggaaccctagagtgtggaacataatataacgacccctgttatagaaggaggaagatgtctcccacacctccttagaggggaggaatgctcaaggaaaccagatcaaacaggaaatgactacaaagggaaacctttaattgagcacttagtgcatgtgccaagcaccttgtggggggcatcgcctcacagaactgttctccccactgtattgatgagcgatcagagtctcagagagcttactgcacagtaaatggttcagcccgattagaaccagacaagctaattcaggagcttgttctcttaacaagtatacaactttggaaacaacaaagtggcatttacgtgtgggagttgttattagaatagtccagcagcatcagacctagaaaagatctgagaagcaatctagccacacagagccaggctttaaatcctggctctgtcgctcactcccggcgtgacctcaggcagcatgtcccttcacctctctgtaccctctacttccctgtctataaattggggagctcatgagttggctataagggttagatgagatacatactcaacgcatggtttagcacatgataagaatgcaataaagagtaacaactattattatttataaacaacacgtttagtctccatataagaagtggtcgtgtctcctgggtttggaactctgggctatctgactccagcgtcttggcttttctggttttccacctgtttctccagtaccattggcagatggtggcagcatatctgatgggggctgtgctcttggcaggagcagggtggggggggctggtccctagcaggtgcccgcactacccacccccttagcgtcagctaagggcctgccctggtgtagccgggccgtggatgcaataccccctgtgatggctagctggttttcagcaaagcttgtttggaagcatttttttcattaggggaattccccatccctcccgtggcttttggtaaacaattactaatgtgttccagaaagcattcggtcccagctgcgataaaatcacgcttaataattattagcatttctgtagcactttggaatctgccaagtatgttgcacatatgttgtctctgttgattctcgcaactttaggagggcacctttgtagatggagaaaatgaggctgggggggtggggacttgcctcgtgtcaagtggcgggtgggacagaggtagggttagaacctgttaacgtctagccagtgtccgaactcaccactgccgggtgctcccccagctcctcacccagcctccaatcggagcccttccctccgggagctacagattggcatctaactgcctcagtactctacctggttcagccaagcaagggcgtcgctctgtctcacgttaaaggggaagatttggggtttgcacgctccaagtcttaccatctccagggtgctcttttgttaaggaactcattaagtcggccccgggttcatttctttgcgaaggaaactctgtggctcgtttcatgctttccctgagttgacctatttaaggaaggggaggggtgtcctttgagaaccagcttcccagcaaaggacttttgtgcccgttcccatgcggtgaccaagtgatgtgtaacttctctgcctctttcttgtccagtgaagacttctttttggacatttggagtgaggacaataggaaatgaacctctgtgctcctccctctctgcactgatacagcagagacctcaggactccagctcagaaacccccacccattgcacttcagcatcattgtcctcctctacgaaacccggagctttttctctcccgacccatttttcctgaagcccctcctacatcccccttacaggaaacatccattagtgtgaaaactagtgactcactggctcctatgactactataaaagtcgatttggaaggactgaggtagctgaagacttaatgagaggttcaaattgacacacgtaggaattagtgaaggcaattacaattagaattcatacctttaaaaaaaaagcatttaagaattttcctggttttttttctttaaggaaaatgtttcataattgtagaatacagaaagcaagtgggagggctttgcgtttgaaattccggaagccctttaaaggcagtgtcaccccaactcattatcctgtatttatgtgtgaaaaggactcaaaggaatgcttaatcccaggctggggggatctacatctgggattgtgtagcgcttttggaggtccccaaattactagcaatagtgtttatttcagcaaacctttatgggaatataaggggaggcttcaggtcccctccagacagcgtcccttgccctctggctgggtcaggtaccatctttctcttaccaagggaaagagaggggaagtccccagtgtagacagatcacccctgagtagcaaattatgtctacaaagtactacaagggaaacagcctctcaagacccgcaccctcggcttccctgtatggctccgtgacgcctcttgtcatgctttactcagtggtctgcaccctctgttcaagctgcagatcccactgctccatcgctctacacattttgatgaagcacctaccgtgtgccaggtccagtgctggcctagggtttttttttttttttttttatgatagtcacacagagagagagagagagagaggcagagacacaggcagagggagaagcaggctccatgcaccgggagcctgatgtgggattcgatcccgggtctccaggatcgcgccctgggccaaaaacaggcgctaaaccgctgcgccacccagggatccctggcctagggtttatggcaatggacaaagcaaacaagatgctgcctccatgaagaccgctgttggttgcttgatacgggaagtaggctgtaagcagctgtttgaaaaatccttgcccttcagtagatcctggtcgttgctgagggtgtgaaaggaacctttctactgtttgagctctccttgcctgttgccccttcaactccccctccttttttttctccctacccccagccccacccccttgggttctcagccttcgagaattctccttttctcccgtgagagagctgacaaaatgttctcttgccccgtgcggggccttgccagcatccacggatcagagcctgtggggtgtctgaagccttcgtccgaggatggcgaccacgtttcacacttttgcttgggccagtagtctggggagatcagaatcctctcctgatggcctctctctaatattagaggatctttggaagaagaagaaatgtttaggctggaaagccacacatcctacccatccctgcgatactgtgttggagacatctagtttccgctgctcagctttctgtctttggaggccagggccactgatattggtgctaacttaaatatggttgttccacggcaaccctttctcagctgatgtttttatcattcacccttggtttcacagcttcttagaactacagtccggtaagatgagaacaaagcactataggatttaaaaaataatagttttttataaaacttctaaccctctgcccctgagactccctttccagcctcccggttgctcttgtcttagggcacaattgaagggtgccgtgactgggagctcttgcttgcctcacataccttcagcctgggaataaccgaagccaagtatacttttataatgagtagtagttgaaagagcctgctctaaaggtcgcctccagacagggggctgtgggactttaatttctttccctccatccctcaccattctgggaagacacatctgggtttccaacccctcactctccaccccggaccacccttttcagagtagtgtttgcatagcctactgagcaactcacaagatctcgagaaaaggccaataaattaaaggaagtggaaatccaactcttaacaccttcagaagctaagtggatgactaggaagcagttcagtagaaatggggttcagtgttcatcttggcgaggcagacatgaatatgagtgtgttgtcctactttctaaaggccccccaaaatcatctccctgttcacccagcttttggttgaattttaaaattttatgtatagatttattctagtttttttttttttcctcatttagtcatttgaatttaagaaatattttggctgggcctcctgggtggttcagtcagttggacatccagctcttggtttccgttcaggtggtgatctcacagcggtaggatcgagccctgcctgcatcaggcttcatgctcagcagtcagcagagattctctccctctccccagtcacttgcactcttatgcacacacacacactctctctctctaaaatcataaaaagaaaagaaaaaaaaatcataaaaagaaagaattcggccggaggggaggagtgggcgatgggataaatgcatgatgagtattaaggaggacacttgtgatgagcattggctctggtatgtacgtgatgaatcattaaattctacacttgaaaccaatatgacactatatgttaactaaattttaagactttttaaaaaagaatttgcattgtaataattcaggcaattcaggaatagccaaagtagaattgtctcctgactcccttcttgtttacctctgtttgttacaatttacaaaaaacaattcggtgcctgtccttccatacttgttctggtgctcttgcaaatatgttttatacacaaaagtgcacacgcatatacattatatatgcgttatgtgtgtgtgctgttgtacacagaaaggatcaccactttattctgtgatttgcattctttgcttaaaaatagatgaatgcatatcattccaaatctgcactttaatcttattcttttcattgcccatcgttttgtggtatgacggtaccataatttaatgcaacacttctttctccctattgggaaattttcaggctgtttccagtattctgctaataaaaaaatactttgctgtgaagaagtttctttgatatgaatctttatgcattcctgtcagataaacccagggtatgcacatttaaaaatgtctaaacactgctattttattttagtaccttaatcttattaacagatcattattgctaagatatatttattcatgtactgtacttaacaccttttgttacatctgtgcttttctgttagaccatctcaagttgattacatttatggttttttatttggggggaaaaaaatctttcaacctccggcttagctttactgattatattcacttaattggattttacctccatgcagttagaaccaaaaagtcattttgggaaatttgactcagatattctccttacagtatcagaccaaaccttctcataaatcataaatttatttctagaatggtctctgtccattaaagctggcaaatgtgtaagaagttgttcagtggctacttgatggttatgaacctgaatggtgaagttcagtagctgagctgggggagagattgcaaattctaaacatttggtaaatgcatgagacctgaaacaattactgttgccaaaactcagtgctaatcctctcctggtgatttctatgcagcttgtttatttagtgaaaaaaaaactaataagagaaacgcagactcccagaggtcagtttacaaaacaagaaatacaatttgcaaaaacactggggggaaaatactcttcagccttcttcaaattgtaaacacattgatgattttggccacaatttgatggactaaccattctcattcatccctgtagggcaacttagttatacgtatcaaagtccctaaaaatatgttttgaagtaattctccaggaaatttataagaataagaacattgaaaacagcccaatcaaaaaagtagagtcaaagttaagttataagagacgccggggtggctcggtggttgagtgtctgcctttggctcagtgtgtgatcccagttgggggatcgagtcccacgtcgtgctccctgcaaggagcctgcttctccctctgtctatgtctctgcttctctctgtgtctctcctgaataaataaataaaaatctttaaaaagaaaaagagttcaattatgagaatataaattaatacagggaaaacatcattttatatcgagaatatttagaagatttaatggcacagggaagtagacactgtaataaggaggaaaaaacagccttcaaaattatggtggtatttcccagaatacctcttaggagatgtgagttcctccatggcctccaaattagcttgagaaatgctcaggtaatcagaattaagataaagtactttagggctggccttcttggagagtcatagaacctacaggtggcttgtatttcccaaatttctttgaccttgaaactttctctaaaatacttattttttttttaatttttatttatttgtgatagtcacagagagagagagagagaatgaggcagagacacaggcagagggagaagcaggctccatgcactgggagcccgacatgggatttgatcccagatctccaggatcgtgccctgggccaaaggcaggcgccaaaccgctgcgccacccagggatccctctctaaaatacttattagcgtctttcaaaatgctcaggttccatggaacatagtataggaggtgctaatatgtagttgagtctcattttttatttttaaaaatgtagcacgtctgtcttacatgtttacatgtgtacttatgtttatttgtatgcatgtatgcatagaaaattagagaaaacatatgaaaagggttgttttgggggttgtgaaattactggtaatatttttttctttaaacctgtctgaatttcccagattttctagggggaagaaaagaacacttccaatattgaaaaaaaaatcgcattgtttttaaactttcataaagttaatttaaaaaaaaaaacactggccaaaaaaaaaaaaaaaaaaccactggcaaggggcatctgggtggctcagttagttgagtgcccaactcttggcttcagctcagatcatgatctcagggtcatgagatcgagccccaggtggggctccaccctcagcgcagaatcagcttaagattctctctctccatctctgcctctcttctctgctcactcaggctttttcactctctagttcaaagaatgagaaacagttgtataaaaaaaaactctaggggcgcctgggtgattcagttggttaagcatctgccttcagcacaggtcatgatcccaaagtcctgggattgagccccacatggagctctctgctccgtggggagtttacttctccctctgctcctcctcctcactcatgctctgtctcaaataaatagataaaaatgtttttaaaaaaagaaaacccaagagcaggggaaactggtaaggaatggatgtgtctgtggcctttgtggtggtggtggtttcactggtatatacttatccccacccgactcagcaagttgtatacatgaagtatgtactacatcgtacatgacaaccacacctccatagtctggtttcaaagaaaaagaaaactcaagctttgagtcagatctgaattcagcctgtcattcgtgattgtgagcaagatatttgatctctgtgtcttgcttgcaaagtaagggtacctccacgagccgttgccagaatcctatcaggtgccgcaccagtgtgcccataggtgcttagagatgtgcaaaggtggtttgaattgcattttttttttttttgctagctattacatgcacagggaaatgcctgtttaaatgaagtttaccgttttttgctgtagaaacagacttctgaaatgatgaatgaaatgctcttgttctttagttaagaagctggttccttccattgaaccagagggaaatgttcctagactaatgtaaatggtttccatgcaactttgagtcaggatcctttcctttctctacttgctcctagcatttgtcaaataactgtcataaaggcgaggttttacttaccaacgttccattatctatgactcagaccccttcctatgacttccttttccccaaagatccttaggtttaaaaaaaaaaaaaaaaagtgaatctagtctgtttctcatgggagtgtaaaattcctcattaggaagagttttcttttaaaatctagtacccacgggcacctgggtagctcagtcggttgagcatctcactcttgattttatctcagttcgtgatctcagagttgtaagatcgagccccctgtcaggctctgtgagattttctgcctcccccgtcccccactctctcaactaattaattaaagcatctaatcgcttttaagtgctaagatctagtgccaggagtggtagactttattgttaaagtaggtttcctcactccctccatagctgtcttttgcagtgttgggttgttggtgttttgggtttgggtttttttttttttgttttttttttttttttctcacttctgtatttgttatatagcaactctatagaaagaagcccggtctggccatcacgtttgcaaagctgcctcaaaatttggacaaaaaccgctataaagatgtgctgccttgtgagtacccagtgtgaatgcctccctcccctcctccaccgccaccagcccaggcctccccccgcccccccaacacgctgagataatgtttccctttttaaaacagattgtctggtattctggaacgattctcactgtgtctgtaaacaccaccgtctttattgccgccatgtcagactcggaacagttggggaatcgtgttgatgctaatgaggcgagagctgcagatggggttgcgcactgaactcttgtttacttggcaggataaaaataacgctcgggataatcggcaaatttttttgttgttgtttttctcccccgcagatgacaccacccgggtcttgttgcagggaaatgaggattatattaatgcgagttctgtgaacgtaagtcaagaacctggaccccggagcacgttgatcactgtcacgagcgcgatcgtggcacgggtggggcttgccgtggacgaaccccctcatttcagcccctcagcagccactgagggaaggcagcgtagtcccattttacggagggcaagactaaggttcagagattaaataatttgaaaagtcacaaagctcacggaaggtgagaccagaacccggatccccaaagttcacatgccttctggcctcagctgtggacattttgggcttttttattttttctgttgttctggggttttggttttttttgttttcttttggcccttgccagtgctatgcttgtaactttgtgtttccacccccctcccacattattaccgtacatcccacctaaaaataaggggtaaaaaatctgcttgtgcatataatctcaacagaaaaggtacaggatatcacatgttgtaggaagggcattcatttcaccctgaaaatgcatcgtgggcagcttacttgggctctctgaatctcagtttcttcatttaaaccagggtcagtacactttttctgtagagggccaagtgataaatattttggtttttgcaagccgtggcaaaatggaggatattacttaggtactaacatgaccatttaaaacaccaccatgtaaaaatatgttttacacactctgagcttgcagggcacctgggtggctcaaataggtgtatgactcttgatttcatctccggttgtgatcctcaaggtcatgagatggagcgctgcatcaggctccacatctcacatggggtctgcttgagcttctctctctctccttctgcctctgttccctcccctgctcacactctctgagtaaacaagttaaatctttgaaaaaagaaagactctgagcttgcaaagtggcagttacatttggccctcaggccacagtttgccaacccctgctctaaatgaacagaggatttctcaagttcttaagttacagcaaagtatcaagaactaccctaaaaaaatgggtcttatcagtgattccatttattccaaaacacctatggaattcattgatcctcacttcaccattcctggtgtcttgacagcagagcatcagaattggtgaggtcagcaaggtcaggtgatccaatctctctgatcatggggatgtagggacactaagggttgatgaagccatttacccaagcatcaaccaaaccatgaattaggcgcctctgtttgccttccccacctaagaatgagtgaatcacagtgtcacttcaaagctcagcaggttctgcgtcgtttttcatagaaatgactcagttcagagggaagaagtcaaagcagaccaaccaggatgggggagagacactgaggacagttgaaaacattttgacgtttaacgttaatcaactcgagtggtaactgaggtggtagatgtgttcattagctagattgtggtgatcatttcacaacatatgcgtctatcagagcgtcgtgttgtagaccttaaacacatgcagttttgtgtcgatcctacatcaggaaggctgggtggaggtcaactcagtgacacaagcgttggcggccccgcagagagctaagtgcagcatttgcggtggcataaacctgtatgggctgtgcttcaaagaaaagtaaatggattttgatagaaagccctgtctggggaaagatttggagcatgagggctggtatgtattgctgaaaatacagatggccactatggattatatttaggatacagcagtaaaggtcattaaaattcttgccctcttgtaggcacatttcttgattattgcctcctgagtgtcaaacagcataagtgcttaatgctagtgttacgccaagggtctttcaaggatttttagaaatagccttttctttgattttccttattggctgtaaattgataggatattctgatgtgactgacatcaatgatcaacttgaagagtctttaagatccagttccgtgtccaccttgtcgcagtgacaggtactaatgccttaacaatctcttgccagatggaaattcctgctgctaaccttgtgaacaagtacattgctgctcagggtcccctgccacacacctgtgcacaattttggcaagtcgtctgggatcagaagttgtcactcattgtcatgttgacgactctcacagagcgaggacgggtaagtggcttgatttccagttccagtgtggccatgtcacactgtgctctgtggaacctgaatctcagacctggaaagggtcttaaatgtcctctggtccagcctgtaaatgacccataagactcctttacagagctactgataggcacctccagtgatgggcacctcagcactcctgctccctccctggccttccctgagctcacgagtgatccaacagcccctcactcctagcttagatccatcataacaagccagaaggcaattccccttgcctcccctcccctcacctgccccagaacccctgtgttgactatattctcagccacctaggacaagcgctggtcctgaaaagaacagtgtggggtgccacagtggccataaaaaagtaggagaagttgtaacctctgtcagccttcacttgggttgcctcaaaacccgatgcacaaagaggaataagggcttgattaggagagtgatgggcaccacactgtgcttagcgggcaggcatttccagaggagcagatcgctggttgtataacagaagtcactgtgcaagggatacctgtgttgtgcatcctctaacttgtgcatgcatcaacataacttcattatgaaacatttattcagaaacccattccaaaatcagatataaattaactttgagccacctgctttgaatgatttgtgtgcagcttcctccgaggcctttctccttggtacatagttcggaataccaaattgatccaggctgaaaatcaaattatgcaggatagaacgactagtatgtttgagaacagtctacactctcacctgaaacagcaaaataaggattcctacttattctgcagagccctgtggattgcaaaggacttcagtgcgttgtcccaggcagagggctcccgactcttgcaaaaacacctccagggtccatcctgtgggtattcagctctaattaaaaaaaaaaaaaaaaaaattcttccttacaactagctgaaatccacatgactttggacctttaccccatgtgcttctcccacttggaagatgcagaataagttacccctgtcttctcgtaactggccttttaggtatctgaaaataatctcagactttgtgaaagcttataacccacaggccctgcttcctccacctgcccttgtgggtcctgccctgcctactgacgtcccacctccctgccttttgttggcatctgccctgagcctgagcccctctgaggtctccctgtgaccgtgggctttgtctcccagccatctacctgactgagcgcatcccagccggcccccggtcctgagtcagcattcccttcaccatctggggccctggggacttagccctcctgtttatcgatcatgagaaacctgttcttctaaaatccaggctgcacgtttcctcctcctgctgttacacaccctcggctgacctgagccctttatcttgaggtcccttatctggtcctcaccaccacccatgggttctctccaggtcagcagcttcaccccgtccttgttctgagagatgaggctttcagcatgccccatgtgctttcaccccccacccgtgcatttagcagagcgggcccccccacaggcatggggctggccggagtcccctctcacggctgcgcccagcaggcatgtctgctggtgcagggtctcctctagcgcctgtgtcgccctgtagcctgtgaggggctcccactgcagcatcactttgagttctgccttttctcatgcctacccaaaatgtttttcattatagtctctcttcaggttcacaggtttcctcctagagaaaaaaagacctatggagatacggcttttctactgaaaaagcattctcccgaaccttccatcagggatgcttcctttgagtgaaaattatttcttagatctatgtgtgcaccacttctgatacgtatgatcggcggcagattcacactcagagcctccatttactcatatgcaaagtagagacaccaatgctgcctacctcgtagggttttttgaggcccagctaatgtccttaaaaagtgccatttagcgctgggcatgccctgttcaatagctggtaggctctagcatcctcaccccccacagccagcattaacatggagacggagactcaaaagaagtctggtgacttgcccaggatcacacagttggttagtggagcttattattagaaactccgggattccacagacgccttgctctttctactacaccctcagtgctttgagatgcaaacagacctaaaatctaaattggaccaaagggtagagtctagttctggtcacattcactgggagtctctggaagtaggtatttgcatctctgccttgtatgggctgatcacttcagagacccttcccatcagctgtcctgtgggcaggacactcgggagtctgtagcagagagctacgccacctcatattggtcctaccctcccgggggctgcccaggtgtgctctgcacccccacccctctatgcagggggtggggagaaactgaaggcaggccaccccaggttggcaggtggcagttgtaataagcaaagggaacttccaagtctagtcttgggcagcagcaagacacatggatccctgcgattcccgataaatcttaaaagtttataacgagagagaccctatctatgtatgtggtatgggtgtcttcagcaccacattacttactgtcttaaggctgtgcccttggagcagcctctggcaagcagaacccacattctccggtcaggggacggcggagggttgagagggtgagatgcctctgagtgctcagggccagttcacgggccacagcgcagtcacagccttttgatgatgttttcaaacaccctgtgaggatgaggcaggtgtagaccagtgggtgtcgactctgccttgggttgggggcgcagccgccccttcatgtctaagggaagttgggaaatggccactaaccaaaacacacccatatttaaggtttacaagttaattcttaactccagctctccagccgcatattttaatttcagatattcactgtgacgttcagaaataagaatagaaaagctgttctagacaaattacgtgcatctgttggtttatagccccttttagctgtacacaaatataatttttatgtaagatgttgcaactataggcttggaggaaaaaaaatttgaatgagtcagaatttgatggaactatgatgggatggacagcttggtcctgagttttgctttgaagccggaggcaagagctgaaacagctgtggcccaagcgtcaggatttggaagggaggatcaaacagagggagaggctgtgggctcagggagccggtggggaagccacagcaggccctacgaccctgggcaagtcgcttaccccctgtgaggccgaggttccctgtctgccatctgggagatgcgtcagctccgtgagtcacggacaatggcatctgagggtctaggggatatggcttgaatgtaacggatgttcatctcagtttggagacttctttcactgagatttctgaggaatttctgatcatccttagtgaagttgatgtggggcccgtagcaacaatgataacctgccccccacccccaccgcacccccgggtaagcctagtactacacactgttaacctcccgcctctgtcaccacctgggaagcaggtgcagtccccatctatcaggcaaagacggtgaaaatctgagaggtcgagtagcaggttcaaagtcacaaggctaaccagtagaggagatgagtccaggggcgctctgacttccccctgtgtgctcttaacggctatgccttgaggcctcctactcgaagaggcaggggatggatggtgaaggagggtgaggcgaaagggggtgtggagagcagagaaggtggggcctgtggcctggagtgggcttgatgggggatggggatgaatgaggaggagaggtcgctcctgtatcaagacaccctgtctgaaaaaaaataaataaataaaaataaataaaaataaaaataaaaagacaccctgtctgagacctctccagttcttccagcaacgggacccgcgtaatgaagaggatgagctctggactcctcctccaggctccagccccagccctggcaggcccacccccaccctgctctccaggggaggaggggaggctcctctgctgccctcaggagacaggcacagagcagattggctttgcctgagtctctctccacctggagctctcacggtccctggtgtgagtgtgagggctctggccaaacaccccacctggccgtccaggtgactgctcatgtgcaccccccacagcggaggtcagctcacccccagtggttcccttgcagaccaaatgtcaccagtactggcccgatcctcccgatgtcatggaacatggcaactttcacatccgatgtcagtcagaggactgcaccctcgcttacgtgttccgagaaatgctggtcaccaacactgaggtaagcagcgcccctgcagggactggtcgggcacagcccagcttagaggcaggtgccacatcagcatccaccatggtcccggatgatagggcagtgcttccaggtctgttccccatttttacctggtgcacatttcggtaaacgtacaagccgacttgtgaaagaaccagcatccttgttcacagcaccactgttgacactagcccgcaggtgggaacatgcgatggacattgacgggtgaaccggtgaacaagatgcggtatatttatacagtgggatcaattcagcctttcgaaggaaggcccttctgatgcatttatagcaggggtaaaccttgaaggcatcgtacgaggtcaagttagcccgtcgtgagaggaaagtactgtgtgactgtggagtacttgaagtagtcaaaatcagagacaggaagtggaagggtggttggctgcggggagcagcgcatgaagagttagtgctttgtgggtacagagtttcggcttgagaagatgaaaaagttctggaaatagtggtgatggttgcacaacactgggaacgtatgtaatgccactgagctgtactcttaatagttaatggcagacttcacgttatgtatattttgccccactgatggcaagtactgtaggagtggaaggggagaaatcatagtgggtgcaagaggagaagcaagaggtcctgaggggggcaggcccgagccgagtcctagaagatgggcgcgtgggagagtagccgtggagggtgagtgggagcttgcaggggcctcgacagggttgtgcacagtgggcgcaagggcagcaggagaccgcaccaccggagctggggccaggcccggagcctgccgcccgccttccagattgactcaccaaggccgccctgcatcttccgcagaccggggaggagcacacggtcacgcatctccagtacgtcgcgtggcccgaccacggcgttcctgatgactcctcggactttctggagttcgtaaactatgtgaggtccctgagagcggatggcgaacccatgttagttcactgcaggtaccgtggctggtcttgtttgtttgaatccacagactccgtagggaaagacctggagagagttggcaaggatcaccctcaatcccccgagaccatatgcttttaccagaaggctctgtttactttatttcttcactatttcaaaagattcgggattttacgaaagcgtaagtctctggatgtagaccagaggaggaaagccccaccctccgctttccctctctccttgggtcgcctcacatgagatgtgagagtggggtgcagcctgcactgaaaggtgggtttcttttcattggtgtttgagcccctggcctcagaatatatgaggagaggccttctcagcctgttggagagcacgccctgaatgtgttttgtattcttgaaggtcaggggaggaaagctctgtttgagtaaacgtgggaactggctaggagttaagctgggccgtgcagaaatacggcctgagcaaatcccaagtctccgcttctgtccccccagtgctggaataggtcggaccggtgtgctggtcaccatggaaacagccatgtgcctaattgagagaaacctgcctgtttacccactggatattgtccggaagatgcgagaccagcgcgccatgatggtgcagacatcagtgagtagaaagccatttgataaaagtagtaacgatgggcgacgccttctgagcccttgccgtgggctggacctgccgaagggttttgcaggtcaagagaccaaggccacagagcttgtcagtggctgagcgatccccaaagcccaccccttcactgtccttcccgtttccctccctccatgacgtgtttcctgccaaccccatagccaggcagcagtcctggttgcattttctttttttaagatttttatttattcatgaaagacatacacacacacacacacacacacacacacacacacagaggcagagacacaggcagagggagaaggaggctccatgcagggagcccgacatgggtctcgatcccgggactccaggatcatgccctgggccaaaggcaggcatcaaaccactgagccacccagggatatcccctggttgcattctacatcgtcaagcccctggcccatcatttatccatcagaagtaagcagaatcacccgaagattttaccccacctgcctgtctgtgggtgagccttttggcttaatggatgtttcacatggccctaccctctcctccttgggccgtctcccgggtgtgtcacatctgggtaatggacactgcaagtgtcctcaagaggagatcttgtggttttccccctggaacttcaagagaagcggggggagggggaggtgatgtagaaaaaaaagagcatgattccggctaccccacatattgggtgacctttggatggtcccttaacctctctccaagcctcaacttccgcatctgcaaaatggggaccagcccacattcgtctagaggattgcacacgttggaaagacccgggcttgtatgtggtgggaaaactggattagtccgttataaatgtcaacggtaatggaggaagtccaggggggagtctgagaactcaatggagagcagcgaaagccgactttggaaagtcaaggaaggctttctggaggagtgactccagatgaaggaggtggagtcagcaaagaaaggagcggaaattccacgttacagccagctctgtgtccgcacaagctcatggacagagcgtgtaagatttctctaataattgtgagctcttgttttatattatactctggaaatatggttaagaggaaaaaatcagacactattgttctctgccctgtttcatgttatggggacggctggttaaaaatgtttacagtgcatcctggtagatcttcctagctgctgaagcatggtctaaatgctttctttcagcctgtacacgtcactaagtaagttccttaggtgttggtgaatagaaagaaccgtatcctacagagcagctttcaatggccctccttaagacaaagtccagaccccttaccccggcacaaggccctgcacaaccacgtcccagccacctatgcaagccctgcctcccaggccctggggttccaggcaggggctctgagccctgactgtgccacgcggaacgggctgtcggagtctccacacttgagcatacactagaatggtcaccctgcagggttgtgtccccgacccgcccccccagacccctggtccctccctacactgtggtttccgccatccattgcccagccctcacatgtaggcctgacggcttcacgggtgtatctgctccacgggtgcttccctggcgtgtagcacagggtgtggcctggcccagagctggacctcagggcatatttatgagcgatgtgaaagagtgggcatagatgcgaaacggtgcagtgttcatggagctgtgggtggaccgacagcttgcttttctcttcttttccagagccagtacaagtttgtatgtgaagcgattcttcgtgtttatgaagagggcttagtccaaatgctggatcccagttaagacaactgtgaaaaacgttcattcctctttcccaagggcatcctccttaaggacagaccttctctctggaagcagcaagagggatttgtaggctgtgggggaggaatgagcacatttgaagccgggcactttaaagttctgtagaagaggtatcgtgtacataggaactgtgtagatacgcatgcagttacagcaacatttaggcctgttattcctcagaaataagcaaaaggggatctcagtttggggcctgtcctatttgccttcctccctagacattactatgttcctgtaaaccatccttgggcagttgagaggggatgccagcgatgctgctgaccttccagaaacaagagggtgcggcgcttcggagttgcttggatgttttgtgtgtatatgtgtataggactctaagggctgagctttctgggcttcagggcggagctggaagcattggcgttcacctgccgatgctaagggcaccctgctggatgtactaagtggcagggtggggggaattcaggctgcagggcgggcagtaccctttctgccctgcgttcctctatccccactccctcccacttcccatactggtgtttc

SEQ ID NO: 84 - DNA, 323165 bp, Canus lupus familiaris
  misc_feature (41665)..(41858): n is a, c, g, or t
acaatggactatactactcagccatcaaaaagaatgaaatcttgtcacttgcaatgacatggatggagctaaagagtattatgctaagcaaaataagtcagtcagaaaagacaaataccatatgatttcactcatatgtggaatttaaggaacaaaacagatgaacatagaagaagggaaagagtgagagagagagagagagagagagagagagaagcaaaccataaaaggctcttaagtataaagaaaaaacaggattactagaggggaggttgggcagaggatgagctagatgggtaatggttattaaggagggcacttgttttgatgagcactgagtgtcatatgtaagcgatgaattcctaaattctatacctgaaacaaacaaattgtcactggggaaaaaaattttttaaattgaattttgtattctgcatactgtcactaagagtcctctttcaaggttcagtcagataacattttcagatatcctagataacaaataggatgtacaaaactttccccttctgcatgattctagaccttaataacctgcttggagcctattattttcaaaatttggtcctatcttgctggacacagcctgatccttctccctgctgtgacatgttccatttgtcactgcctccatgactgcctagaaagtcacacatcaattccaccaagattccacaaacactttcctgtctcattgttaccttttgccatactgcaactcccttctcaactattcctgaagcatgggttcaggccttcagatcaccctggattcagatttcaggcatgagaaaatgccaggatctccccagggggggaaagggattcaaaactactcagcatctcagtttgccaacgtgctctggattctcagataaaaaggaacctgactttcactgtatttcagtgatgttatagtggggatgaaaaaaaaagcatgaattctaaaaaattctgtttttagaattctaagaattctggttaagtgatttgacagaacttggtgactgtttctgtgagaggaagggagaagttggggatgactcaggtttctggtttggggaactgggtggatactggttcaccatcttttatcttcactgttgcaatgacttcctattagttctcgccgtcttttaaccttatattgcctttctaaaccacacattttattatatcacatctctgcatttaaactttccatgacttcatcttaactgcaggataaagtctaaatatttttgtagcagtggagtttcctcacaatccaattctaaacttaatgtcctgttccacacacatcaacttactctgaccaatcatctgctaaacatacacctctgaacatggcattccctctgtcttcagtgttcttagcactatcactgcctagaacaatttgatcagctttcagagtctcattgaagtatcacctgttatgtagaaatggagtctccactaggctctaatatcaccatgtgtagatcttcattattgatcttctgttactgtaccacaatttgtttacatatctccatcattaaaatacaatgtaaaccatgtaattaacttctaatcttattttaaaaaggaggtagtagacagtttttaggtccactagtcaggtattccgttttattccacttacatacttaattgactatttcaaaattttcaaaattaagaaatatataatttacatataaaatttaaaatataaatttcatgtgcataatgtaaaaaattaagttacattattatttattgtcctattgtgttatattttggatgttgttgtttgctaatggactaggaaggagacactgacttgggggaaggaatactctctgaacatttcaaagagaagtctccttcttaatgaaagaggagaacctaccctatttcttgtatctgaattctaagaaatagtgagatgactgtgtaactgcagggtgtggaattaaagagaattgaagcaaaaaccttttcccaacataagtcaaagagtcaggaagagaaatagagagtggagtttgcacctgggatgagagaattggagcaagtacatgagttcgtgcaaggagcgattctctgaaacttccggtggaggaaacccattttttaaaataaattttttattggtgttcaatttaccaacatacagaataacacccagtgcccatcacccattcactcccaccccccgccctcctccccttctaccacccctagttcgtttcccagagttagcagtctttacgttctgtctccctttctgatatttcccacacatttcttctcccttcccttattttccctttcactattatttatattccccaaatgaatgagaacatataatgtttgtccttctccgactgacttacttcactcagcataataccctccagttccatccacgttgaagcaaatggtgggtatttgtcatttctaatagctgagtaatattccattgtatacataaaccacatcttctttatccattcatcttttcgacaccgaggctccttccacagtttggctattgtggacattgctgctagaaacatcggggtgcaggtgtcccggcgtttcattgcatttgtatctttggggtaaatccccagcagtgcaattgctgggtcatagggcaggtctatttttgactctttgaggaacctccacaaggtgtatactagagtggctgcaccagttcacattcccaccaacagtgcaagagggttcccctttctccacatcctctccaacatttgtggtttcctgccttgttaatgttccccattctcattggtgtgatgtgggatctcattgtggttttgatttgtatttccctgatggccagtgatgcggagcattttctcatgtgcttgttggccatgtctatgtcttcctctgtgagatttctcttcatgtcttttgcccatttcatgattggattctttgtttctttggtgttgagtttaataagttctttaaagatcttggaaactagctctttatctgatacgtcatttgcaaatatcttctcccattctgtaggttgtcttttggttttgttgactgtatcctttgctgtgcaaaagcttcttatcttgatgaagtcccaatagttcatttttgctttgtttcttttgccttcgtggatgtatcttgcaagaagttactgtggccgagttcaaaaagggtgttgcctgtgttctcctctaggattttgatggaatcttgtctcacatttagatctttcatccattttgagtttatctttgtatatggtgaaagagagtggtccagtttcattcttctgcatgtggatgcccaattttcccagcaccattgattgaagagactgtctttcttccaatggatagtctttcctcctttatcgaatattagttgaccataaagttcagggtccacttctgggttctctattctgttccattgatctatgcgtctgtttttgtgccagtaccacactgtcttgatgaccacagctttgtagtacaacctgaaatctggcattgtgatgcccccagatatggttttcttttttaaaattcccctggctattcggggtcttttctgattccacacaaatcttaaaataagttgttccaactctctgaagaaagtccatggtattttgatagggattgcattaaacgtgtatattgccctgggtaacattgacattttcacaatattaattctgccaatccatgagcatggaatatttttccatctctttgtgtcttcctcaatttctttcagaagtgttctatagttttgagggtatagatcctttacatctttggttaggtttattcctaggtatcttatgcttttgggtggaattgtaaatgggattgacgccttaatttctctttcttcagtctcattgttagtgtatagaaatgccactgatttccaggcattgattttgtatcctgccaagctaccaaattgctgtatgagttctagcaatcttagggtggaggcttttgggttttctatgtagagtatcatgtcatcggcgaagagggagagtttgacttcttctttgccaatttgaatgtcttttatgtctctttgttgtctgattgctgaggctaggacttctagtactatgttgaatagcagtggtgagagtggacatccctgtcttgttcctgatcttaggggaaaggctcccagtacttccccatttagaatgatatttgctgtgggctttttgtagatggcttttaagatgttgaggaatgttccctctatccctacactctgaagagttttgatcaggaatggatgctgtattttgtcaaatgctttctctgcttcgaatgagaggttcttggtttttctcttgctgatttgtcaaatgctttctctgcatctaaaatggttcttggtttttctcttgctgatatgatgaatcacattgattgttttatgagtgttgaaccagccttgtgtcccggggataaatccgacttggtcatggtgaataattttcttaatgtattgttggatcctattggctagtatcttgttgagaatttttgcatccatgttcatcagggatattggtctgtaattctcctttttggtggggtctctgtctggttttggaaataaggtgatgctggcctcatagaacaattttggaagtactccatctctttctatctttccaaacagttttagtagaataggtatggtttcttctttaaatgtttgatagaattcccctgggaagccatctggccctggactcttgtgtcttgggaggttttagatgactgcttcaatttcctccctggttattggcctgttcaggttttctatttcttcctgttccagttttggtagtttgtggctttccaggaatgcgtccctttcttctagattgcctaatttattggcgtatagctgttcataatatgtttttaaaatcgtttgtatttccttggtgttggtagtgatctctcctttctcattcacgattttattaatttgagtcttctctctcttctttttaataaggctggctaatggtttatctatcttattaattctttcaaagaaccaactcctggttctgttgatctgttccacagttcttctggtctcgatttcgttgagttctgctcgaatctttattaactctcttcttctgctgggtgtaggatctatttgctgttttttctctagctcctttatgtgtaaggttagcttttgtatttgagttctttccagtttttgaatggatgcttgtattgcgatgtatttcccccttaggactgcttttgctggatcccaaagattttgaacggttgtatcttcattctcattagtttccatgaatctttttaattcttccttaatttcctggttgaccctttcatcttttaacaggatggtccttaacctccacatgtttgaggtccttccaaacttcttgtcatgatttggttctaatttcaaggcattatggtctgagaatatgcaggggacgatcccaatcttttggtatcagttcagacccgatttgtgacccagtatgtggtctattttggagaaagttccatgtgcacttgagaagaatgtgtattcagttgagtttggatgtaaagttctgtagatatctgtgaaatctatttggtccagtgtatcatttaaagctctcgtttctttggagatgttgtgcttagaagacctatcgagtatagaaagagctagattgaagtcaccaagtataagtgtattattatctaagtatttcttcactttggttattaatcgatttacatatttggcagttcccacattcggggcatatatattgaggattgttaagtcctcttgttggatagatcctttaagtatgatatagtgtccctcttcatctctcactacagtcttcggggtaaattttagtttatctgatataaggatggctacccctgctttcttttgaggaccattcgaatggtgaatggttctccaaccttttattttcaggctgtaagtgtccttctgtctaaaatgagtctcttgtagacagcaaatagatggatcctgcttttttatccagtctgaaaccctgcgccttttgatggggtcattaagcccgttcacattcagagttactattgacagatatgagtttagtgtcatcatgatatctattcagtccttgtttttgtggattgttccactgaacttcttcttaaaggggaattttaagaggcccccttaaaatttcttgcagagctggtttggcggtcacatattctttcagttcctgcctgtcttggaagctctttatctctccttccattttgaatgagagccttgctggataaagtattctgggttgcatgttcttctcatttaggaccctgaatatatcctgccagccctttctggcctgccaggtctctgtggagaggtctgctgttaccctaatactcctccccataaaagtcagggatttcttgtctcttgctgctttaaggatcttctccttatctttgtaatttgcaagcttcactattagatgtagaggtgttgaacggtttttattgattttagggggggatctctctatttcctggatctgaatgcctgtttcccttcccagattaggaaagttttcagctatgatttgttcaaatacatattctggccctctgtccctttctgcgccctcgggaaccccaactaaacgtaggtttttcttcctcaggctgtcgtttatttcccttaatctatcttcatggtcttttaattttttgtctcttttttcctcagtttccctctttgccattaacttgtcttctaggtcactcactccttcttccacctcattaaccctcgtcgttaggacttctagtttggattgcatctcattaaattgatttttaatttctgcctgaatggatctaaactctgcagtcatgaggtctcttaagtcctttatgcttttttctagagccaccagtagctgtataatatgtgcttctgaattggctttctgacattgaattgtaatacagattttgtaactctgtgggagagaggactgtttctgattctttcttttgaggtgaggttttccttctagtcattttgctcagtgcagagtggccaaaagcaagttgtattgggaaaaagagaaaaagagaggagagaaagaaggaaagaaaagagaaaaagaaaaaaagaaaaaaggaagaaaaaaagaagaaaaagagaaataaaaagaaagaaaggaaaaaaaggggtgagggaagcaaacagaaatcaaaaagaaagaaaaaaaacacgggggagtatcttctgattctgtacactttaagtcccttgacttcccctggaacttgtcggtctagctggtcttctgggggaggggcctgttgtgctgattttcaggtgttagcacttgggggagctgctctgccccctgcctggtgcagggctcagtgggggttgtttaccccgtgaggccccaggagcaacagccctagtggcggggccagctctggagccctggagtccgcccccgcaggaactccggagctctccgtctgcagggcctggaggctccggggcggggccgctgatctgctcagctcggggcaggagcgtccttggggtcctgggccctcccggcctctgcctgtcccggggggggaggctggatcctgggctgtgtcccggcgccctgtgccccggggcctgcgctgctggattcgtgctcccgccccacagccccctcccggagccgcccccgagccccccccagctgctcccgccccgcagccccctccgcggagccgcccccgagccccccgagctgctctgggtcccgccgtgcgcgctgcagcccttagggagctcggcgcatctcccgggggcaggtgtctgttagtgtccccaggagcccgagggcatccccgccctcctgggtcctgctccacctctccgcgagcccctttccccccgggaaggtcggtgcagctcctgctcctccgggacggggctctcctgtcctggggacactcgccccggcctcagcccggctcctcgcggggcccctccccctcggaggccttttgtgtctttatttctttttccccgtcttcctacctggatagaagcgcgaactcttctcactgtagcattccagctgttctctctttaaatctcaggccgaattcgtagattttcaggataatttgaaggttttctaggtcatttggtggggacaggtgatttggggaccctactcttcctccatctcgcccctcctccctcaggaaacccattttgttagagtgtctgtagccctgcttacggatgcaagcaaatgggctactaagtgatccttccgttcacagcagcttgagggtaacactttcccagatcttcttgtaggcagtaggctttcatttctaatggcaaagtgtgctgtgaccactaactataattttcagtaaatttcacacatgtatatgtttacagagacagacacagagggaaattgatttattgcaaggacttagctcagacaattatggaagctggcaagtttggaatctgtagggcaggctggcagttaaagcgcttgagtagaggttgatgctgcagtcttgaggtagaatttttctctgggaaacctcagtttttgcctataaaacttagactgccaactaattggatgagatccatccacattgttgagggtcatttcctttacttaaagtcaactaagagatgcctgagtggctcagttggttaagcatctcccttcaactcagaacatgtcagagtccagggatcaaggcccatgtcaggttctctgctcagtggagtctgcttctccctctccctctgtgatctctcactcttgctctctctcaataaataaaaatagaatctttcttttttcaaaaagcataatttaaaaaaatcaactgatttagatgaccacatctacagaatacaatatctatatagtatttgattaagtaactgtgtgctatatagcctacctaagctgacatataatactatcaaagacagtgtggtactggcacaaaaacagacgcatagatcagtggaacagaatagagaatccagaagtggaccctgaactttatggtcaactaatattcgataaaggaggaaagactatccattggaagaaagacagtctcttcaataaatggtgctgggaaaattggacatccacatgcagaagaatgaaactagaccactctcttgcaccatacacagagataaactcaaaatggatgaaagatctaaatgtgagacaagattccatcaaaatcctagagaagaacacaggcaacaccctttttgaactcggccatagtaacttcttgcaagatacatccacgaaggcaaaagaaacaaaagcaaaaatgaactattgggacttcatcaagataagaagcttttgcacagcaaaggatacagtcaacaaaactcaaagacaacctacagaatgggagaagatatttgcaaatgacatatcagataaagggctagtttccaagatctataaagaacttattaaactcaacaccaaagaaacaaagaatccaatcatgaaatgggcaaaagacatgaacagaaatttcacagaggaagacatagacatggccaacatgcatatgagaaaatgctccgcatcacttgccatcagggaaatacaaatcaaaaccacaatgagataccacctcacaccagtgagaatggggaacattaacaaggcaggaaaccacaaatgttggagaggatgcggagaaaagggaaccctcttacactgttggtgggaatgtgaactggtgcagccactctggaaaactgtgtggaggttcctcaaagagttaaaaatagacctgccctacgacccagcaattgcactgttggggatttaccccaaagatacagatgcaatgaaacgccgggacacctgcaccccgatgtttctagcagcaatggccacaatagccaaactgtggaaggagcctcggtgtccatcgaaagatgaatggataaagaagatgtggtttatgtatacattggaatattactcagctattagaaatgacaaatacccaccatttgcttcaacgtgggtggaactggagggtattatggtgagtgaagtaagtcaatcggtgaaggacaaacattgtatgttctcattcatttggggaatataaataatagtgaaagggaatataagggaagggagaagaaatgtgtgggaaatatcagaaagggagacagaacgtaaagactgctaactctgggaaatgaactaggggtggtggaagaggagaagggcggggggtgggagtgaatgggtgacgggcactgggggttattctgtatgttagtaaattgaacaccaataaaaaataaattaaaaaaaaaaagcccacctctttcaacttggcactcatacatatatctttaaacaaataaaatctgtaaataaggagagttacaaagtcatactcctgtctaacataatacaactatcctgcaaacaatcaaaacaacacttttcccccaggacaggatataaagtccttgggtgatatcccttcttctcagtatcctgtaacttaatattgtgatataaagttaactgctcttcatacagcttatgcttaatgatgaggggaaaagggagataagaaaagagcctagtgtgaatgtatagacacctagaaggctctctgatccatgtaagtctaacttggcaatacaatttttttgtgtgagctcaagtattaatcttgatctgaagccaaacattatttctttttctcattaatttggtaagcaaataagacataagactattgtcaggtttttctttccagagtaatttgcatatcatatgtcaaacaggaaattgcaggcaattaaaatgtgcagcaataaattgtaagtcagactccagaatttaagcccagactcaggagacatcaaaaagaagtagacagtaacaactgaagatacaacagtgaacaagatagatttggtcttgtgaagtcctaccaagaggttggaaacagcttgtcaaagcttatctagaataagagatggcatgtgaatctggaggctaattaaaacttgttaatatcacaatcagtgctttgtaagtctgacttttattggttgtgcttattttcctttggtaaccccttagagtttatagagctacttagaatcctccagaacttttcagatgtgtcaaatataagagtttattcctgcttaggttggagacatctagggagcatcatatactatgagactcagggtaatagaggcaacaataatcacaggataaatactgcctatgtccggaaacaatgaaatgaggttgctgtattaatgagcatgtagtggaaaaatctgggtgagatttattccaggaatgttaccagaaagagtgagttgaatattccaagaaaagagtaagttttgagaccagtctttagagaagtatgtgtaatactgaggaggaatggaatattcaaaggccttgagccatcatgaatattatgataaaagaccacttcattgcccataaaatttaatccccactgttaagacatattacagctagataccatgatgtatttttcaatgtagatctgatctttatttttaattaataatacatggcagcaatatctgagagctgatttattatttatgtatctctttcaaaaagtcattcatccattatatattcagagaggaaattcctcttattttattgaagttagttttcaatagaaaatacattaagatgttatcatataccctgcaaaggaaaataagtataatgaaattcacaattctcccagatgtaggcatgtcctgcatactgagagttaacagcagttaaatggatcagactgacttttcacagaagctttttcttaatgttaaagatatttatttaaagataagaggccagtccatgtgtcaatttagagctataaataagaattaaattaaggaagaatagaagcattttaaaaaatgtatttccctaacctatttgtgttcttggtttccagtaaaagctaatatgattacacaagcactgaacacgtacaaaaatattttaacattatataaaaattctattcaaattgtgttcaccagcacttaaaatgtaaaatacttcgcccaattttgtcaatataaaccatcttccaggttctccaatggcttagaatgttcttttaaacaacaaccatgtagtattactattaatttcttttcttatcaaaacttctcagtaacattagagaaagtaaattagatttagagcaaacatacaactgaaaactaaaataaaacagtgggagaagtatgctgcaagagagagaagaacttatcatatactaatctgtatatgcctgagaccacttcattcgacatttatttaacccttctatggacaagctacagttctaggcactcaggatacatctttgaaccaagcaatagtttgcctgtctcagaggtttcattcagcctccatgtggttggaatttttaagaacagtaagaacaacaataaaaaatgaagaaatcttcaatcctaatactggtagtagggaaatgttgaatattgttcatattcatgtttacttttcaaaaacttattgaacagagcttttcagaaagtgttacaataaacattttgcttttcccctacaaagttcagaggaagcattattttgattgagacatttcaaatgagttaggtcttcttttgacactctttacaatgccaagcatcaaattcaaaatggaggcattcattcagttattcagacgtcttacagattcacgtgaaatacaaaaacaatttttgtattaattagcttaggtcacaatgcagcagttggtaaattattatttgggatgttgcagaagccctggatgacacttaacaaaatacagatttttttttcacaaatgcatttccttttttatttatttatttttccatatgatcagaaagtgagtcagccctctgatgttttataggggaaaaaaggtgttcctttgaaaaataccattatgtttaaaatttgtgccattactttcttagagcagatcctaacctatagttaaattattcaattaaaataacatttatccttaattactgaaataatgtttgctcaaatcagaaaattcagaaaatactgaaaagccgagaagagaaaattctttaatgcctttcataagcactgatagtagttttgatgtgtgtctttttttcctgccacactttttgtcatatagtatatataatttcttatcatatcatacagcatatataactttaaactctttttagcttgccttttttcttggcaatcccaaatgccttcctatgtcaataagtgttaatctattgtggcatttagattttttgcataagtattaaaatctttgaaatgtattaaaataatgcagataattctatttattttctaagagtaattattgaaattgggagttgtggacaactgccatgctcagtttaagggtttcatttatgtgtgtgcatgtgtatgtgtgcatgtatattttcccatattttccagaaagccatgtagtactttgtccctagggctatgaccttccattgtctttctaagcctttacaacttaggtcaagatttctccgtgtcacttcatttaccattatcttgccttcggtctcacaaagagaaataatacaataggataactccctcaactcattccctcatctgcaaagttatttatatatttgtttcttactcttactttttctccttgctcagaagctttgagtggtcttcccttatgttgtgaactctatcaccttagtacatatctaagggcatgtgccaacgtttgccaaagaatttgttcactttatctacctgattacatttcttggcaaataagtgtttttcattaatatatttttattgataataaatattgatagaatctctaatatactttttaattcctgaaattgatcatttgtgttctctaattttttcttcatcagttatgctagaaatttgtcaattttactatttgaaagaactaaggtttctattggaaagttttccttttgcaattttgttttttgctttaattggtttctattcttatcattgttatttcctgttttatatattctttgaatttagtttgctgttcttatgtcttgaaatagatgcttgaattcttgaatttcagccattgttaatttctaataaatacatttagggctataaattttcttctaaggaaggctttagctgtatccaagaatttttgatatatcatattttaattttctttcagttttaaatattttctagttacttttgtgattcttcttttacccatgtgttatttctaagtatgttgctgattttccaaaatatttggaagggtttgtctacttcatttttaacttcattttcatttttagcttctacactgttatcagagaaaattctctgaattttgtaaattcattagtactttttgaaattcatttcatagctagtatactgtattttggttaatggtccatgtacatttggcaagtcaactctatggtttacactggatttctgtaaattggatcaatattgtgtttctattaattgtgcttttcaaaacttgtatgttctaagtaacttcggtttgcttgttccagctacaacaagggcatgtgctcaaatatctcatgtctgcaaagatgactaagtcttcgagttctgcctattttgctttatatattttgaaggtagaagattatatgcaaacaaatttaacattgctttatctttctgctcaattaaccttattatcattatgaaatatctctcattatctcttgtaatgcttttttctttaaaggcacgttgttagatattaatactatagattacaccattttcttttactggtgtttatatatataaatatatagaggtatatatacatctatctatctatctatctatcatctgtttataaaattttattgtgtcctcaaatataaggtgaacctgttacaattaacaaaaattgtattcttttaaaattcaatctaaccatttttatttcattgaaatatgaaacttacattcattcacatttaaagcacaaacatttgtattaaatctaaatcttattacttattacttattttctttgcatcctgtcttttaaattaatttttaaaaacactccatttctgggtacctagctggcacagtcagaagagcatagaactcttaatttcagggttgtgagtttgaaccccacattaggtatagagattacccaaaaataaaaataaataaaaatattccatttccattccttatttgatttacacttacgttacataatttttgtttttttattagtaaatgcttttgaccagaagtcaacaactgtgacccatagcccaagtataatctgctacttgcttttgtaaataatattttgctaatagccatgtctgttcctttactcattaataataaattcatttattcattgtttatgacagctttcatattacaaaagcagttggatagttatggccaagattatgtaatgcacaaagactaaaacagttactccaaggttctttagagaacctgcttgctgagccctgctctcgacttgaacatgagtccccaaactgttacagtctgtcttaaattagtattaccatttcatggacaaggcaaggttttcagaatgtttggatactacttctcctcattcatgtattatttttatcatgtgttgtatttctgtacatattcagaacgtcaatagatgttataattactatctaacataaatactcatttagatttacgccaaactggtgcgccacccagggatcccctaagacaatttttcttatccaaaattaacttcctttagtatttattttatttttagtgtactgggaataatttttccagtttatttactacttttgcctgaatatgtctttcttttgtttttatatttaaaaaaaaaagattttatttatttatttgagagaaaaggggagtgtatgagaaggattggggcagatggagaagaacagggaatagctcactccctgttgacagtgagcagggctaaattccaggacactgagaccatgaactgagctgaaggtaggttcttaactgactgagccacctggatgccctttttgtatttattttgaatgatatttttgcagattacattattctagattaaaaaaacattttctattatttaaaagttgatacttcattatttctggacttcaatatcttctgttgtgtaatcagcagttgaaatacagttgttcctttgaaaatgatgtgtctttttgtttggctgatttgtgtttgcttctatgatattgttctgagtgattttctctttgtttttcatcattggctttacagaagttgttgaatttgtgatttgatgcctttcatttgttttagaaaattctccaccatcttatctccacaaatattacactttatctttcctctcttttatcacttctttagactccaatgacttgtaggaatttttatcattgtgtcccctttcctttttttctgtgtttcagtcaattttcctatattcccatttgccaagaccttgatctgtacttaaatcaatctatttaattattaatttcaggtaattttcaagttttagagtttagtgctttttagcattaagactttttaaaattattttatagaggttccattattctgaggcaattttctatcatctctatttgtaatcatagtatttatgagtacttaagattcaaatctataactcaaaagctatatcaacaatgtgtgtcttcctattgtcttgtgtgtgtgtttcttctcttggttttggacatttgttcctatttcttgacaagactgttagagttcattgaatgccagaaattactaatgaaaaactatggaagctgtggtttatatcttcatgtatagaacatttctttaggggatccctgggtggcgcagctgtttagcgcctgcctttggcccagggcgcgatcctggagacccggaatcgaatcccacgttgggctcccggtgcgtggagcctgcttctccctctgcctatgtctctgcgtctctctctcactgtgtgtctatcataaataaataaaaaaaaaattcaaaaaaaagaatatttctttcgtaggcaaactgagtaagatcagtcacctttgactagataaggctagtctatttccaatttgcagttagttaaaaaggacagctcttctggattgtgagtggaaagcctgaggtgtttccattggcttttctctttggcaagccctgaactcgaatttctgtctcctcagcacttgaagaccacaagaatccctgcttaattttttgccatttagcagccactttctgccataaaactctgtccattcacaagttaggaatttgcaaatgtatctttggaaaattgtgatctttctattggatccatttttctgtgacttgtttttccaaaattttagccatcaatcctagcagccttgataatcttgaaccttgaattgcccagtaagactgctgcaagctttaacttacttcctgctgttctacacctatattttatggtaagaatcaacaattccccaaaaggttttgtggcgtctaatatagtttcctgctctcctgtgtttccatcttcctgaggatttggttccttacattcagtttgtcttattctgcaatgtcttgaaaaaacagcttttaaaatacttatttagatttacacttgtaattggtcttgggaggtgggggtgaggtgggagtttggaggagaagtcatagtctgaacccagttactgcaacaaagccagaagtgatattccttcttcagtgatttctcctttaatttaaacttcaaacttctgctttcttgaggtttattttattgtcctttttcttactcattaacttgaaagctttctttatctgcttgcattctttgttgtgtattttttaaggctatatatttttcttctgagttttcacttcagctataaattccacaggctctgatttttgctatgtacccaattcctcttgcctatctcaccatcctgaatgttttgaaatttccctttggatttcatactaaaccaaaagcatataagaaagaatgtattaatttaaaacttctagatgctgaaggttttttttccggtgttgttactaattccaactttattgcatcctagataatgaatacattatttacattttgcaatttgttgaggcttttctcagtttaagatatgaatttaatgagtgttttcatggtgcttgaaaacaaaatatattctctattattatttacttcctttactggctttgggctttgttctttttttagctcctttaggtgtgacattaggttgtttacttgagatttttcttttctcttgtaataggcctatattgctataatcttccctcttagaacagcttttgctaatccaccattgtgggtttttttattcaccattgtgttttcatttaatttgtctctatatatttttaaaatttactctttaatttcttggttgacacacttattactttaacatattgtttggcctcctgggatccctgggtggcgcagcggtttggcgcctgcctttgtcccagggtgtgatcctggagacctgggatcgaatcccacatcaggctcccattgcatggagcctgcttctccctctgcctgtgtctctgcctctctctctctctgtgagactatcataaataaataaaaatttaaaaaaataacatattgtttgccctccatatatttgtgttcttcggatatttttttcttgtgattgatttctaagttcatcctagtattgttggaaaagatgcatgatattatttcaatctttttaaatttattgagacttgttttgtggcctattatgtgatatgttcgggcaaatgttccatgtgcacttgaaaagaatgtgtattctaatgtttggggatggcatgttctatatatacctcttaagttcatctgattcaatgtgtcattcaaaaccactattttcttgttgattttctgtctgaatgatctatccgttgatgtgagcagggttttaaagtctcttgctgttattgtattactgtcaactcttttatgtatatattaataattgctctaggggattcctgggtggctcagtggtttagcgcctgccctaggcccagggcatgatcctggagtcccaggatcaagtcccacatcaggctccctgcatggagcctgcttctccctctacctgtgtctctgcctctctctctgtgtctctcatgaataaataaataaaatcttaaaaaaaatagttgctctatgtatttagatgcttccatattgaatgcatagatattaacaattgttatatcctcttactggatttttccctatattattatgtagtgtctttctttgtctcttgtaacagtctttgttttaaagtcaattttgtccaaaataaatattattattcctactttgttttcacttccatttcatggtaagtggattttcgtctttaagcaattcccatctgtatgctgtctacaaaagatttacctcagacccacattttactttaagtaagtaagttaggccaattatcttgcttgatattttagagatttcagtttaaatttcttattattttatactatgccttctatttatataactttgctttaaaaaatctttcactctgtagccaatttgcattatttcctctggaaactggaagatatttttagtatatctataaatttatacattgctttatattctatgtatttagtttgcattacctctgtattattaacaatgataaaatgttcattcttctcttttcaaccttctcccaatctaacaaattgaatttactatattatttttggctttgattaacctatattttaaaaaatcagtattaagagatattatttgacttccagacactataaatgaatgaataattgcaagtatgtgcatgcatgcacacacacacacacacacacacacttctactatgtagtgacattattgttactgaccacttttgttttgcaattatcatatacatacatgctgatagcaaacacatgctactctttgcttaagaatttttcctggtcggttgacaaagtagtttcttaattaatgctgagaaaattggtggataatcacactaactcccctagtccttgattgagaaaactccaatgcttgttctgtactctatcccaaagcagcctagtagaatcaacccccagttgcccattacttaaggatgcacctttaatggcaacttcccttatctgcttgacttcctcattctctacaggtgcttactaggattatttttctttaaaaaattgcttgcactcaaatctctgagtctacttctttgggaacccaatttgagatatacttctatcctcctttatctctaaacttccattagttatatatcatttcttttccctacaatacatagaatcaatacttactactttaatatttcaatctattttttatttggctaaatttcagtctacaactagtttttttcaagatctcaatggagtaatatttccagaggtcttttattttgaaaaaaatatttatttattgcatttatattaaagaacagtttgactgtgtataagattcttaggtcaaattaaatggatagcatcattgtcctcttgtgttaaatattactaaagaatattacattattaatcctcttttacatatattaatgtttaaaggaataactttcatccttgaagtttagaaactttgttacattatatccagttttttttattctcagatatgttatgctatttcaatgtatagctgcaatattttgatacctggaaaattttcttgaactaaatcttcaaatatttatttgttcatttacttagatttcattcttttgggattacaattatgcctatgatgaatagttttcttgtagcttccctagctaagcatttatttccatttttttttttaagaatctctccaccctgtggagcttgaactcatgaccttgagatcaagagtcatatgctctgctgactgagccaggacagtgcccctccttccatattttaactcttcatttctattccattcatcttattttatttattcctatttttttattctccttatatattttcagcagtttccagttttttggtattaattccaatttttatttcagttataattaggttttttttccttcaacttttttttagcataaatggcattgtttcttttccttatatctgaatatctcattcctgagatcttgtatccattctttatattcttaatatataggtgaaatagctactatattaagattgcagaatattagtgacaagatatttggtttcaatgttcatctttccatggcaatatttttctgagtttttatcatccgacttgcttttctttctctcttttttactcataatatttttgtatgtattcatctttatatgaagcaatatttcttgctcatgcatttgagaaatttcacttcgaaagggacttatgatttgatctagattagcaattattttacatgtgatgatacagaattaaccttttttcttctaattcaacaggtaggagatatttaggtagatctttttgtaatgcaaatgtcttacatcctccttctcctatctattacgtgtacagatctcttcttcttaatgggacttctctaagaaattctacatttggctctatttcctctgcttttcaaacatattcatgtcagggaaatttatttatttatttatttatttatttatttatttatttatttatttatttttgccaacctttccttctctgtgcaacaattgcaaacaagagctgtttgcattcaatgtacaaattttaccttaaaaatagtatctgctggatttttggtgaccaactactataaatactctctctgtacttttttccctccacaagttctgccaacttgtaagtttgggttttgttgaattattctttatttgaatctggattttacctatttcttgcttttcttgatgcattggttggtgtccaaattgaaaagggcgagctgctaaattttcatcatcaccttcagattggatgtgtgatctatattttgatcttaaattatcagattttagatttgaagagcataaaggaagcatagaaaacatctggtccaagttttattttgaaaaaggtgaaattcaggctacagtaaaatgatattcccaaggccaataccaaacgtattataaatagcaaaataaaaatcaatgccagatctcttgactatcagtctaatcccgtttatgctatgtgaactattccacttcaaaacttctagtgaaacatgtcttaaagcaagtgacttgttagcaagttaggaaaatatttgtaaatgttagaaaggtggacaaattttctcaataatcaagataaatttttaaagattttatttatttattcatgagagacacagagagagaggcagagacacaggcagggagagaagcagactccatgcagggagcctgatgtgggactcaatcctgggtctccaggatcactccgccgggggctaaaggcggcgctgaaccgctgagccaccaggcctgccccaaaagattattttcaaccaacatttcttctataataaaaatctgatataggtacagattaaatattactttaattttaggaattaatcatgtaataatctcagcttttggaaattatggtgttattaggctgctattcaatattatggtggccagtagacctagaatgtttgattctgtgttgcctgctatcactttttttcagaagttatctgaggtaaacaaaagctatgtgcacttaattgtcatttgagactttcctggagatacccatgaatttattctggacatgtacactaccctgtgacttagaataaagtgatttctttccctaatcctaaattttaatttattaattcagtagcagtaaagagggatgaaaaaagtagacaatatctctgccttaatagagctaacatttattccaggagaaaacaataatcagattagtagggacatagatagataaatagatgatagataattgaattttgccatgaaaaatgaaatgagggagtaagtgaatgtagttccctcatatctctcctgttactcatgctcatatttgaagagctctggaggctagaggataatagttttaaggattgtaaaactgccatctcactatctgcagaccatttcaggttagagatgggcagtatttcaggatgcagggcctaccttattagcataacatggacttacattgcactgtgcttacatgttttgaattttatttcataatgcattggtgtgctattgtgtctgattgaagtgccaaagcaataagacttctcatgagctactaaaagagtgatggagatgggaacaacattaggtataacggaaaaaagaatcttctgtagtgatattcaagttgaaatataactgagggaaaaatgaaagcctacaaagacttcaaggtagggcataggaacagcaacaggcttcacatgtttaaaactcagaaaggaggagagtgaagctggaatgttaagtggaagacagagttttgaaagataagatttggataatgggaaaaagtcaaatcatagaggtccttgaaggcataaaagggatatggactttatactcaatataataggaagccattagtgtcgcttttgcttttggttttggtttttaggtactccagcagattctaaggccatagaaatgaaaatactgctataatccctgttctcaatgtctcaaaacatcaatagctttggtttatagtatgaactttcatttcttattgtttcctcctacctgaaatcaaatagtttaacttttttgtgactgtatatacctatatgatttaaattattgggatatataaacagattatttcttcttttctgattccactttaatggtatctataatcaatttctctattcagtttaacctctcctcttttatcccaattgcttggccaggataatttttataaccttttctttttttttttttttttgtttttttttataaccttttcaagggagttttaactcagaaatctgaataaatgatggctatgatcttatatatggcaatagttcccatgacatagttctgtccctttgatgcctatgctgtattttaaattgaactgtgaataggataccatgcttaaatgactccaatctgtcgataaccaattatgttattttctgtgccagtgttatccatgtatctttataaaaacatttaacttatctattgccacataaacaaattaccccaaatctaatgggttaaaatgttacttcccataattctgcaatctgatctgggcttagttaggtggttcttctgttccatgtggtgactgctgatgctgaatgtccaaaatggcttatttactcacatgtttcactcctcagctgaaggagtatttggaataactagggactggctggatatttttctttatccacctggtctatccatcaggatagctggatgtccttaagtaatggttctgggttctaaaagaaagcattacaatatcaactccaatgtgcaagctcttattaagcctctccatgtatttagtttgtcagtgttccattggtcaaaatcaaatggtcagtccctgagtcagtgtggaaaggaattacacaagggcatgaataccaggactggtaccttagtgggctgccattataatagctaccatttccaagaagaaattctatttatcttgtgataatcacatgcttaaatcaggtttactcaacactgaatgtgaattcagtaaaacatctattaattctccagcatataatacatgactgcatggttacaataaatggctgtaatttggacaaggactagggaaatcattaccatgaacacttgatacagtgatccaaggttcttcttcctgagaatctttcatttaatgggttctgatcttgtgaagtggatcaggtttaaaagctaattttcttaaatatagttggaacaatctagattaaataatgtctttgtaggctgcctggttattttatcccccaggggtcctgtgtcctattaatcatcaccatagaattctgcagataaagctaccctgtttgtcatgccaatctttcttttatttacaattattgtgcccattttgttctggtggttaaaatgttggattgcttgtttggggattctatcaaccctttactacctgggatactccttatgtaaccctagcctttggatgacagctgctaactaagcttcttaacaattctggttcctctctcagtaacacattctttgtcactttgataaacagtgttctctgggcctcttcataaaacagtcatctcatgagttttctctctgctgtaatatatcatctttagcaagcctatctgaacttttaatcccccttactctactatggcagttctacttcatctagtatgagccatcaatttctctaagcttcacagttccaactaagcagtgtattatcatcatttccatctcctgaggctttaggatattaaatactatatcttaagaaggtgaatccatattgagaaccctctattattcaaatttatgcctctctcaatttgattcacctccttcaggatccagtcctgaatcttgctggcacatgttgactagatcttccatcttttgactaatagaccctttcttcctgtgtgtgtgtgggggggggcgggggctgctatccttttttacccggtcatattgctgctggaccctaattattaatctaatggccaagagagaagatggagatgaatcatgtggagggatgaagtatcttgttttctaaggcacaagattgtgcattgtgaagcaaggaagaagtgctagcctttcatggagacatggtacacttcctaggcatggagttcagagaaatctgacgattaaaagttttaaagtgtatcgacacagaaatccaaatatcaccatcctaagtttcagggtccattccttcctagtcagagccctgatcttggcatagcaaatttgttggaattgagaatttgaaattctctgtagttctgctactcataaaattaagtcgtggacctgatccttagccttctctgacctccaggcacaggggatgagaatctcttcattagttatatccttccctaggagactctcagtctttcaaaatttgttttaaattgatgattaatcaccctccaccatttgttttcttctttcaataaatcaatggcttttgctacagccatccagttccatagtccttatcattactattttcctcaaatctctcatatgtctgagatattgcagccatcaatccattcctttctactggcatctcattccagttcactactgaaaaaaatgtttagcagttatactaaaaccatatgccaggagctatcagtgccagtgatgatgtcttcagagctggagtgggatccagctccaaatttcattttagagtatgcttcctagggctgctcctggtaccaactgtcataagttctctaggaaatacatttcgatatttacatataggaagtttgttgagaaatattcttaagaacaacacacatggggtaagggaagtgggattgggcaaaggaatatgttgaacaaagaagcagttgcaacagagacttcaggttatactttcaggagttcttgtgctgaatgacccttcaaagatgtcccaattgggtaaagtatccatcctttttttttttttttttttttttttttttttttggtatccagccttttaatcccacattaaacttccactggagccactcctgaatataggttgcagcttcttttttgccaagggcaattcctagagagaatgcagttgtgagctgtatttaatgctcccaggagctggttgctgcatgccacagaactaaagaagtggctttcaatacactgcagtatgcactactttagctgaccactaaagccataactagacttttcaaactgatctcttcggagtagacatctgttttttaatctgcccacattgcttctctcttccttctgggaataactttcaacttttcccaggaaattatttccttctaataaggaatttgtgtacccatcttccaaaatagcctaactccctctccagctgttgttaattgtcccaaagatgagcacatgaccccagcaaaatgaattagcatgctctacacctccttccctaactgcaaagattagcttctaaatctgtctaggtcaaataaagtccttctttatgaatttttgtaatagttttattgagatataactcatatattataaaattcatcctttaagagcatatacctcagttatcattgatatattcatcaagttccacaagcagccctactatctaattttagaatttttcatcaccctcaaagaaaaaacaaccctgaactctttagcagtcactcttcagctgttcctcactcccaacccttggcaattactcatttactttctgtccctaaggatttgcctattctggacatttcatataaataaaatgatataatacatggtcttttttgcctgtcttctttaaattgacataaggttcccatgttgaagtattagtacttcattcttttttattgcaggataatattccagtgtatattgtacagaccatattttctaatctgctcatcagttgttggatatttgagttgctttcactttttgactattatgaataatactgctttgaatacttgtgcctcatagttttctgaatttgataccagagagatcaactatcaatcctactgagtttgcctcagctgggcatttttttaaccttttgaaaaagctattctagaatgagagaaaagaggtgaagggagagaaaataaccctttatttcattttcccaggtacatagaccactgccctccccataccttagttatatgatcaattaaaatatccaattttacataagtagctttgaactgggtttctgtcacatacatataaaataattcagggaaatacatgacttctgataaaatcttaaataatagcagggaagctctttaaatcacaaaagtctaaagtttataaaaatttataggggttccccctttctacctccatctctctgtggaaataacctgtctgtggaaataattcccagtcttaaagtactaaactccaaaacatattttattcagaaagctatctctatctaatgatatataatgatctcttcctttttaggtttaaagaatttgttttgcatctttatttagggttttcttctatgtgaattaaatttgcattagcttacaatctgtcttcttcattagactgaaagctctttgagacctgagatttttcttgttcaactttgtatttctgatagttccatggaaatatattcaacagacacattattttttacctattagttacatattctatacaagacaccgaggtaggcctggggttagaaaaatgaataggatcccataggagcttacattttattatgggaagtctataagtcaataagaatgaattatataactgttacgataaaaataaaaataagtacaggggacatcaaaagatggagccaattctcttcagatcattgaatcttagatttataggcaaatccactcttaagcccaaggaggaaatcctcttcagtttctgacaaacatatgtaatggttagataagggttaatctacactgtatgtgagttcactaactagcccgtttcagattctcaagggagtcacaagtctcaaactaatcggaattgtgatttctagttcatttggacacatctcagagaatggatctcttctgcccttagtgtcataataggaattagataggtaggccttcttctgggaactggaaggatcctagtagactattagaccattctcactgtttatgcttaattaggaggggatgacccaaaaccttgcagaataagtcccctctgcaaagggtgaaaaaaaatgacgcatttctctcacatgtagcttccttctttcttctctaaggttatacttctaatttaaaaaaaaaaagtgatgggccagatagaaattataatctttcagtttgtcactgaaatcatatcccttcttattaagtgcaaaatatatgaaagtagtaactatcaatatagttggtatctgtcaccttcaatcagttttaagaaaagaagattaaaaaaattatcttttaaggctagtgtagtaatgagtaagccacagtaaagatttagaaaatgaagtgaactctctctctaacagttttactgatctattataattttttattagtttgttttagtgtaggttgaggtccccttgtgaaaatagtttcaatgtagcaaggggcacttttcctgaattgactactttagacaatggattttaattactttaagttggttaggtgtagggataattgaacctcagggagagtttctcttactgtaaggctaggccaacttccccatgaccttctctaatctgttaaacatcccatataacccattgcctattctataataatattattccttctctactataatgttggaatattcttctacaattattctgctactataacttgtcttatagcaaacactaccagcatagaaatcatggtcatgatacatagcagatattcaacacataattgctgagtgactagataataatccattataattattttgaacattcacctttttttttttcaaattccttagggtaatttcatggcaacaatttattgtgtcaataaaacaaaattctttaatttttaagttgaatttttatcagtcctgcctattattatactattaaagtttcctcgtgaattatttattttagcatatgaaacatccatccattatatatcattatgagaatattatatacatagaagatgctcagtaaatatttatagagtgaaaaatagcttttctttttaatcctaaatttttaaatttattgtcttctattcagttactcttcaaaagtatttttcagcctcccaaaataaaaggcacatttttttttttaatcggtgggaaacaaatgtaggaaaatttttacaaaatattttcagtgacacaggattatatatatttcttagatttctcagccaatttttcaaagtgtttcctattcacatctgtagccattagatacaaattcactatacccttcactttctgtttttttggccactcaggctacctcctaggaggtagctctatgctcattccttaaactcctattttcaatataattttattttattttattttatttttaaaatttttatttatttatgatagtcacagagagagagagaggcgcagagacacaggcagagggagaagcaggctccatgcaccaggagcccgatgtgggactcgatcccgggtctccaggatcgcgccctgggccaaaggcaggcgccaaacccctgcgccacccagggatccccctattttcaatataattttaaatgttcctctctacatatatttgacctagactcacgaagaaattacccttgcctcctctcttcttcaaaagtatacatgggttttgattccaggaggtcttcatgaagaaatattttttattgatccttaaagggttcttggacccaattccaacctgtgtgtgagtagaagtttccctataacaaccaacagttttcagactccaggggggtgctgcaaattcagctaaattctgacactatttacccggaaatagcatgagattccacttcaggatcctccacttaagacactagtcacaaacacaggttgttacctatgcttctaatccactggtttagaggtttttgtgaccacccccctcctttggttcaaataatttcctaaagtggccacagaactcagaagcattgtacttactggatcaccagttattgtaaggatgaaaacccagactagccatgaaggaggtgaatagggaagttatgagggaagcgacacggagcctccaagctctttctaggtgtgccactctcccagtacctccaagtgttcaccaacctgtaagctccctgaactcagtcctttggggtttttacggaggctttgtttatttattctttttattttattttattttttatattcattgggacacacagagagagagacaggcagagacacaggcagagggagaagcaggctccatgcagggatcttcaggatcacaccccaggctgcaggtggcactaaactgctatgccacgggggctgccctggaggctttgttacataggagagattggttaaaccattggccattgacaaatgaactcctcctccagcttctcctccttccctccctaaaagttccaactttctaatcacttggttgattctcctgaaaatagccccattcttaggcgcttacaaaaaaaacgatctaattaaaaaaaaaaaatgatctaattataacaaaagatactttgtatcaccgtcaacagcccattccaggggctttgggagctgtggacacagaccaaatatataggagaagtatataattggttatctaaatgaccaaatatttagttcttataaattaaattatcacagatcccaataagtattgcctgagatttcccccagcattcttcaaatgttcaagatgcatgatctctatgtgaggtcttgcaacagaatgtagagcctgggccattgagaacccctaccctaaaaactcatagctcactaaggcagcagaggtccttcatatgtgtcctcttaaaaagaggaatttatactacacagccgctctctcagaaggtagggaatgaactctaaactaccaaaggaaaagagcaaaaataattctcagaatatctgcaggagatattattcttttggtattggtttcatgttgcaaacactgttatttgctcttggagaatgaagacttaatttttggtaagaattttcaccgtaaagtgccactttctgttcttattcacttttttctttttggctctttatagagtcaactatgctaactgctttttcttctctgaatgagtgtttcagacactgactacaatgacttgctttttgtgtcatactccaagatgacctagacttcagaagaaagcttacttcacttgcttcacataaatgttctcattttcttctcttctaaactgttaccaggctactcactaccctatgaaatgatgtggtatatgtggaaaataatttctatattctcttacaattattggattgtgtcaagggggcacaattctcagaacctaccctaagcacagtgataactagttacaccactggctagaaacattatctttgcttctctttctgttgaaatttttctccaaccacttcctgttattgtcttcttgttccaaatgctcccaatttcaatttttttttccaatttcaattttttaagtatgatttatttacaatggaaactgtctcaatatcacattacttagaagaatttcttgatccatctgatggtccagaggtgtttttcaattactacagaaccatttaatactactttatcttacagaattctccacaaaaattgaagcaaaatgtctggaaaggtctgaaactttcatcattgtatattcagtgttcagctgtgtcttgcagacggtaggtacttacaaactcttttttgaaagaattaataaatgaataacgaaaacatgaggactaatatctccttttatgaaaggaggaatgaggaatgaagctaactgattcagatactgagagacccgtggggagttaggtgctaaggaaaacatcaatacaaattgtgtagggagtggggctgaaaccctaacctaatccatatgtatagggtagaaatggaagtaaattaccagaggccacaaagacaaccaaacaaacaaaaagaaaaatggagaattttagatttcttaacattcaaacattttatagttttgggtgatatactgagcaggctcccttacaataaactccccttctcttgttagcacaagcaggagtatggaaggggagcagaatcagctacagtataagcaggaaactcagttccacaagtatcctcaacagaatagaaaagaagattcagcacatctactcaatcactagagaacaggagcaagtacataattcaaacagccctgcaggaaggaatatcaagtggctaccacaaccaccaattgacaagaaatactattccatatggacttagtaaattcaggctgaaaggaactgaatatgtcttttcgttattgttcattccatgtcttttatttatttttattgaggtatagttcacatacaattttagttccagctgcacagcatagtgatttaacaattaaagatatgatgaggtactcatcacaataagtttagttactatctttcaccaaaaaaaagggctatagcaatattattgactatatttcctatgctgtactttttatccccttgacttattaatgttatttttttgggggggggagagaaagagagagagagagagagagagagagagagagagaggaagagaaagaacatgggaagaggggcagagagagagaatcttaagtaaggcttaatcccaggactgtgagatcatgacttgagctgaaatcaagagttggacatttgcctgattgagttacctaggtgccccttgatttattaacccctcttgtacttcttaaccccctcatctatttcacctattattcctcacccctctggcaatcaccagtttattctctgcatgtattaatttatttctgagtttttgttgttgtttttattgttgtttgcttggtttttagattccacatatagatgaaacatattgtcttttgctgtctgacttagttcacttagcacaatatccttcaggttcatccatattgtagcaaatagtaagattttgttctttatcatggttaagtaagattccatttatatctatatctatatttatctctctctatatatatttatttatacctcatatcttctttatctgttcatctatggattgagacttagattgctttcagatcttagctattgtcaagaatactgccagaaaaataggagtaaatatatcttgttgaattagtgtttttgctttcttcaagtaaatatccagaagtgaaattagtgggtcacatagtaattctatttttaatttttgaggaacctccataatgtttcccatagtggctggaccaagtgacattcccactatcagtgcacaagtgtccccttttctccacatcttcatcaacattaattatttcttgtcttttttatactaaccattctgactgtggcaaggtggtatcttgtggttgatttgcatttccctgatgattagtgatgagcatctttttctgtgtttttgcccatctctgtatcttctatggaaaaatgcctattcagatcctttgcacatgtttcaattggattgtttggagctttttatataattcagatattaaccccatatcaatatatcatttgcaaatatcctctcctattcagtaggttgcctttttgttttgttgattttcttcattgtatagtagaagctttttagtttggtgtagtcccaatagtttatttttgcttttgttttccttgactggggagacatagccagaaaaatattgctaaattcaatgtccaggagataaaaacctgtgttttctttagaagggttatggtttcaggcctcacatttaggtctcaaataaataaacacataatttttgagtttatttttgtgtaagctatagaaaagaatttcattcttctgcatgtagctgtccagtttccccagcaccatttattgaagagactcttccccattatatattcttgcttctttcattataaagtaattgaccaaaatagcctaaggttattttcagaccctctattctgtgcctttgatctaagtatctatttttgtaccaataccatactgtttttattactacaactctgtagtatagtttacaatctgggattatgatacctccagctttgttctttcgtgagattgctttgactctttggagtctttcattgttccatataatttagagttatttgtcctagttctgtgaaaaatgttgttggtattttgatagggattgcattaaatctgtggattgttttagattgtatggacattttaacaatattcttttaatccatgagcatggtatatatttacatttatttgtgtctccttcaatttctttcatcaattttttggagtataagtctttcatatccttagttaagtttattcctaggcattttattctttttaaattcagttttaaatgggatggttttcctaatttctctgttagttcattattagtgtatagaaatgcaacaaatttccatatattacttttgtatcctataactttactgaatctctttattagttttaatagttttttttaatgtgtgtgtgtgtgtgtgtgtgtgtgtgtggaatctttcgagttttctgtctacagttattgtcatctgcaaatagtaacagtttgactttttgtctttttcttttctgattgctgtggctagtcctagtactgtattgaaaaaaagtggtgcgagtagaaatccttgtcttattcctgattttagaagaaaaactttcagcttttcacgagtaactatgatgttagctattggtttgtcatatatagcttttattatgttgggatatgttccctctaaacccactttgttgagaatttttatcataaatggacgtcaaaaaatggacgttgaattttgccaaatatcttttctgtatctgttgacatgatcatgtgattttatactttattttgttaatgttgttatcacgttaattgattgtggatattgaaccatctttgcacccctggaataaattcttgattgcagtgagtgatccttttaacatattactgaattcagtttgcaaaaattttattgaggatgtttgcatctatgttcaccagtgatattggcctgaaatctcctttttctttttccttttttttttttttttttgtagtgtctttgtctggttttaatattaagctaatgctgtcctcatagaagaatttgtaagctttccttcctcttctattttttggagtagtttgagaaggatagatactaactcttctttaatgtttggtagcattcatcagtgaatccatttggtgctggacttttgttttttgggcacttttaaatttcaattcaattttgttactaataatcagtctgttcagatgttcaatttctttctgagtcagtcttgaaagattgtatgtttctaggaatttatccatttcttctagattgtccaatatgttggcataatttctctatagttactcatatttttaaaaaaattgtacagtatcagttaactcctctctcatttatgattttatttacttgattcctttctctcttcatcgtgatgagactattcaaaggttcataaattttgtttatattttcaaagaaccaatccttagtttcactgatcttttctattgttttagtctctaattcctttatttcctctgtgttctttattatttccttccttctattaactttcagctttgcttttcttttactggttccttcagatgtaaagttatattgttttaagatttttcctatttcttgtgataggacaatattgctataaaattccctcttagaattgcttttgctgcatcctaaagattttgaatcattgtgtttccattttcatttgtctccaggtatttttaattttctctttaatttctttcttgtccttttagttgtttagtagcatgttgcttaaccttgatgtttgcgggtttttttccagttttttcttctaattgatttctaatttcataccattatggttataaaagatgcatgatatgatttcaatattcttaagtttattaataattgttctgtggcctaacatgtgatctatcctggagaatgttccatgtgtacttcaaacaaatgtgttttctgctatgaatattctatatgaataaaatataaatatattatttagacaaatatacattttttattaatgaatttcccccaaacactcaacttatcaccacaacttggtatgttagtttcctattgttgctataacaaattaccacaggcctagaggcttaaaataacacaaatttattatcttacaatttggaagtgagaagttcaaaattaatcttacctgggtaaaagccaagtgtcagcagggctagttccttctgcaggctctgcagggaaccattcctttgccttctccatcttttggaagctgcctgcatttcttcatttgtgtctccttattgatattcaaagcacattgctccaatctctgtttcctcactatgtctcttctctgatttttctgctttcatcttataagtacccttgtgattatactgggcccacctggataattcaggacactcttcccatcccaagatcctcatcttccttttaatgtaaggtaatatataaacaggttctgggaattaaaatgtggacatctttagagagaccattacacaattagattaatattaaataattaagtgtggggaaagtaaaaaatgaggaaagcctctcagtaacaacaaaaaatgttttattgactaggaaattcctttaaaccttctgaatatttcatccagttctgagctagcaaatgggaagtgtcttaaaatcctatgaaaataacatatagggatctctgggtggcgcagcagtttggctcctgcctttggcccagggcgcgatcctggagacccgggatcgaatcccacgtcgggctcccggtgcatggagcctgcttctccctctgcctgtgtctctgcctctctctctctctctctgtctctgtgactatcataaataaataaataaaaattaaaaaaaaaagaaaataacatataattcagtgtctaaagatctggacatgttcttcaaaaggaaaattgtctgttaatatatgaccatgaagaagcaaaatactatttcacttttacaacaggttactgctttagggggaaaaaaagttttgttttttaaaaggattcagaatctaaaagtagaaatgaagccaacaatattagcaaaaacatccagtctgcaaagggcataaggaatcagccatagcaactacaacagggcaacagcatgtccatttattggaaccatgtccatttattggaacacagagtcagcatgaggctcttataatctccactgagtggggtaatgatattgagggcctcaatgtgctgctagaaaaagaatacaaaggaagaagtcaatggatccaggagtttgatatattgagaaacaagaaaccaactgtggctagcatttttttgtcattttgtaacactggtaaaatcagaaatatgtgattacatgctgctggtggcattctgcctgcaaaagcatctcagtgtgagaattactaagtgcctcttccctgagaacaaatgtggaacaagagataacccagcatttcagacagtatcatttcctaaagggagaaagcacaggaaaataaagtcaactgtgactaaagatgtgcttgacactgagaaacaagaccaaatgagtgaaaacatttctaaaatatcttttcataaatgcacgggcagttagaatacacataagacttaggccacagcatttgtgtgtgtctgtgatctctacaattttcctgtctttctgaaaacaatgtctacaaatcctcaaccaaagagtgccaacattataatgatttatgagtgtagttcatgactattcttaaagatatagatgtttatttccatgagattatttcctgtaggaaagttggtaggaaaacaaaagcaacttttgggtatggatacaggtataaccttgcccatgatgcccatgtaatccttagcaatgactttacatgaccttaaaaggaaggagagggttgtaattaagaggacaggctatggaaacagatctaggattgaatttcacctctgttactcaaaacttttaaaccccagattcctcatttataaaatatggttaaagccacattattttgagaaccaaaggacatgaacattgcgaaatacttacatagtgtttggcacataaaatacacaacaaaataaggtagctgttaacagcaacagtagggaaacacaagaataaacagataaaggagctaaatgttacctcataactttgaagaagttatattttcattcagtgcctaaaacaaaaacaaagctggaagaatttttgaactttagtggatgatttcttggtattttttagcatgaagccctattaaacagtttttaaagatcagccccaaattttagacacatttttgctttcatttttccatctaccaggattatattgacacagatgtctaagcgacgatctgaaaatgtacttaattttcctgtgtattttaagttttattcaatgtatattagagatgacaggatcataaaattgtatagctgggagagatttaattgtaattagttaaatttcctcatttagtgactgtccaaggctcagaaatactcagaattgcactggttgcccagaaaacagagcggagagtcaagatggtaaccgtagccttctgattttcagtccaaggctcttcctccaggaaagttgctccatacatatgacagatacaatttgctacctgaagactatccttactcacaaatttttgtgtgcataaagggagtgaacattgtgtatttctatggaacaaactgaaatgtctttgaagatttggacacttaattgggaccatttttatgtcccttcctcttcgaaaccatggttgaaacatcttttgtgaaaaatctatctatacacacacacatacacacacacatgcacacactcacatacatatatatgcaaatgcatcagttaggacattttctccccagtgggttgttttaaaaagacttatacattcctaagaagtgaatttttgaaaatatttaccacaggtttataggtctcctgcaaaccaaaccccttgtctttaccttattgtactgagatctcttagcaaacaggtatttatagaagacaacacacttctcaatatatcagtacaaaaattagataaaatctcttgttaattcaaatacagaaaatcaactcaatgttaataataacattgaaggaaatactggaaagcagaaaactacaactgtgatagtttcaaaattgctgtcatttcacatatttctccaaggtttcttcattatggtataaaccataaaaacaaattgagttatgtgaaaaagtcatcattttggatactaaaaaagactttgtatgggataaacagttgctacacagttctctctctctctctctctctctctcttttttttttttaaagatttcttttctttctttttttaaaagattttttatttatttgagagagagagagagagagaaagcacacagagggagaggagaagcagactcctggcggagcagaagcccaactcaaggggttccatcccaggaccccaagaccacttgacctgagcaaaaggcagacgcttaaccaaactgagccacccaggtgcccctagatttcttttctaaaagagggagagggacatttaacccatgaattattaccaaatcttcaatctcatctccagaaacattttcaatttccaaaggttaatgactaatttagagatccttgagagaaaaagggacatttatattaaggctccgggaacagcctgaggatttccatctccaaagaagcaaacagagtactcttatctggagaacctatttcaatccacaaccaatttttttttttttttttttttttttttttactggacccagttctgacctggagcaggagaaggaaataaactttacacgggattttaaatcgctttacaagttgtgcttctagcctacaaagtctgagcaacatgaaattatttaaaagaatttaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcctgagcagatcaatactttgtaaactggacccagttctgacctggagcaggagaaggaaataaactttacacgggattttaaatcgctttacaagttgtgcttctagcctacaaagtctgagcaacatgaaattatttaaaagaatttaactgtatctgtagctacttctgtctttgcacaaccggattgcacgtttctccctccccttgcccggcgagtgtcttttcggaagcgggagcggggccgtcctaggacacctgaagtcacaggtactgtggctccgaggttccccgcgcgaaaagctgcgcggggcagaggacccggggtgcggggggcgggggcggggtcggggtcgggggggcacccagggcagcctgcgaggcgaggcgaggccagcgcgagccccgggcagtttcacttcctcgcggctccgagcgaggccgggcggcggccccgtggcccgagtccccgggcgctgcgcttccggagcgggcggccacgaggcgccggggcagccctggcgaggggcggtggccgcccggagctcgcccaggaaggagctgaggcggcgccgtccgcgcccgggctccggccgccgagatgctgccccgcgcgtcggtgcgggcgggcgggcgccgcggcccagcgcccccaggtgggtgtcggcgcgcgcgggccgcgaatctgggggggagggggcgggctgcgggggcctcccctggtcccgccgcggctcgggggcggggaggggggcagtgcggccgggacgcgctggccgccagcgtcccagggctcggagccttgcagagccgcggccaccggcgcctccgccccccgtagcccccgtgggacgcatatgagatcttcgcgcgtgctgcaccgtgacgcgcacatctccggggcctggggcctgtggcctggggccgccgcaccttccggccctgcggggagagnnnnnnnnnnnnnnnnnnnnnnnnnnnnnnnnnnnnnnnnnnnnnnnnnncgctcgtgagaactacgagccgtcagagcggtgggatacgtttgatttagggaccacatggacctgaaggcggctgtacccggttctttctgtgtttccaacgtgcatttgtgaactttgcgatgatgctaaaagggttcagttttgcaactgggaagaagccctcacttgggtggcaggagtgctgccgtggtccacagcgagacacgcagccaggaaaccggagtgaaactctgagcttactgaacagtctctcatagcttgccagcagcttttagggcgatgttgcttctcttgcccagtgtatattttcaatttgtaaatttttttttaaagattttatttatttattcatgatagtcacacacagagagagagggaggcagagacacaggcagagggagaagcaggctccacgcaccgggagcccgacgtgggattcgatcccgggtctccaggatcgcgccctgggccaaaggcaggcgccaaaccgctgcgccacccagggatccctcaatttatgaattttgaaaggctgctctttcttaaaatgggaagtgtgtttgtgacaatttaaatgacaacaggaggattagagcccctgatatgatatgttagttaatatattgacatttgtgatttcagcctaacagtttagtttaaacagagcgggcgaaggtttgttcgaacttcttgattgaacggtttagccagagaatgacttttatcccacttgattcccaagaacactttgaggtacttgctactgtgaataatactggagacacgtgacgcgactgttagctcattctgattattcctgtgccttccatgtgttttccccccatttactgtggtccttcccagtagtggtaatttatcacttgtttcatttacctcctttggccacggtaatgaattttttttttctctctaaaagaaactatcttttgttattaacccatgcccagtccttatgagtatgagtttcgagtagtcttgagtacgtgggtatgagtagtcttccaggggaactagttctaatggattcctttaacacatggagggtttttttgtgtgacctttctttctgcttcggacaagtgtttccacaacatggtggacttaccttaaggtgtcacattgtgagaaagttagtccaacttcattttagagagaaagctgagccccaagtagtgattttcctgtggccccataactaacaattacttgaagatcatttatttgttgcttcaccaacaatcctttatctccttaccaaatttgtgtagttaataggagtataaaatggtaagcacaatttcatgcctatgaaaactttatatttctagcagtaggcgtatggtatattgaaatgacatctgtgtgtggtgccaccagaaagatctatgagtatccaacttgccttttaatggaagcaggaaagataggatactcagtactaaaacaaggtaaaaatattgcacattagatgaaaatcccatttagaatgtacttttctggtttatatttaactagataccatccccgaatgtccaaatgagaaattcttcctttgcattattgctaaactggtaattttaaaaacgaatttcatttcaactgctaccttcaataaaaactgtttatttattaaagcacagatatcccccacctcataatttttgtatttttttattaacagtatttgatacctttgatgaagagacttaaatagtttaaaaacaaaaggcatctaccctgtctcactatatgctagattagatgcttatgtataatttgtgagggacatagtttgtctaggtctctctgtcttgaaattcttgaagatttgaagctttttattctgactgcagtcttccaacatagaaaataaccttgatttctccttccaaagaaatagcccggatctgttctggtgactggtaaaacttgcctccttcagctggaaatattccaataaggttatttttgctaagattttaaagagtggtagctctcttagggcatttcattttaatatggaataattctatgatatctaattgcttagggtttcagacttaaacagcaatgtatgtgatgtatacttggaaaattagagggcattttttcaccacatcaatctgaacacatgtcgtaggtgatcatgccctactaatgatggcctctggactcaggccttctcccatcagtctaactcttttgcttatttgctgtttgatcttggataaataactaaatctgtcattatctcaatttatccatttaaaaagcaaaagtggagtgaacagtaacacttatgtgaaagagtggttttgaagaataaaagagaaagtgcatgtgattttcagagtgccatagcaaatagtaattgcttaatacatgttagttattattttaattcttaggtcattggtcagtcagtagtgtcatatttacatataaaagatagagttttattgagaaaataaagtctattgaattggaggaaaacttgaacgaagccatcaagctcccacatgaaataaatgaaggagtgtggcttcctgtagaaagatattgctatatccttagggaaatattgtggttaatagttctgagagaaggagaaaaagaacccacaatgagaagaactaaggataaattgattaaaagggcattagactacaacatttacaggaaagaataaaatgggaatttaataggacaaaatgagaacatgctttattttcctatatcacagagtatcagttgtgacccagttaaaaatatatctgtaaatacatgtggaaaagtaaagatcaaactgaaaattatgggatatttcattgaattataagaagaggagaatctactttaccaaaatattcactgcttgcttaaaaaaaatccaatgtaaagagatattattcctgccaaatatgagtcacaagttttaagaatttcaatagctgttacctgattacctggattacattgctgttttattattttctttatatattacagggtatcttacagtaactttggagatagctgtgaaataagaataggaagggcaaacattccagaagttattattttataaaaataagttgttaatatggccttgtaattttttgccgttataaatataaagaatggatctaagttcactgcagatattgaaatagtccaactggtaaaaatatcggatattatatggaattatacccctcttgtatttatttttcgctcagaagaaaggaatgatttttctttagacatttaccactgaacagaaatttctttgttgttttctagtgctccacattgaaataatccagaaagaccacttccctttttaatcatgagaagtaatttttcaatgtcttgtggctttataagacttcctgtgtatccaattaattaaaataagttatgttttatcacactgtgtgtcatattttcctttcttttaagctcttttactgtttttctgtattagtaattcccattcttcatttttaatatgctctgctttataaagacatcctaagaagtacaagaacagtatctgccttggaatttcatttactaggtgcctcattttttggtcatgatccatgctgaagactaactctcaccctttgtttaggaacaatactccatttttttttaaagatttttcttttttttttttttaagatttatttaaatatatatagagaagcagagacataggtagagggagaagtagactccttgaaggaagcgcaatgtaggactcgatcccggaacaccaggatcatgccctgagtggaaggcaggtactcaaccactgagccacccaggcatatctcattttttgttctaatttagccataggtaggatcatgcctaactttcagaaaaatagcataatggcaagaccacttgaaattctggcctttaagactgcatttattactactttgaattcatatggtacggatatcacattactttgataagtgagatcatcagaagattagcacctactttgaataagaagttatagtattcagtataattagaagaaagtgtgtaataattcatcgtgttatttcatttcctataagaacctttctaaactattaaagttgtaacttctaggtcattgtatctttttaggaaggatacagaaagtggagaaacatccacatcaaagacgtgaggaagaaacataccatatcaatgtagtgtgattctataaaaatcatgtcactagaatgacaaactgtagagtatttcttggaactgtgtgaagaatctctaggatttacatgtttttctcattggttgatctccttttatttagatttaatagactgatatacaatctcagaagacaactggatttttagtacaattagtgaactactgagtcactaatgagaaagtcagtgagataaaattgaagcaaaattctaccctcatttcagttaatgagataatttccttaattctctcataacttttttttttctctcataacttttcaataaagatttagaacttcctctacttcgggcacttatctatggaccagagattcattctttatcatgtcctgtccatacccctgtgaaatggataaaactctgtatcattcatattcctggatacagtcatgtgggtcaagaacaggcactaaattcagaagtagcttgttgcttttgcttactggccaatgatgtgtgtgctgtgtggcttttacagagttgttggacttaatgaagcctttcctttcaaaacagagaatgagactgttgtcagtggaagggtaatgttcaatgacccacaataggagatgacagccagtggactcttgtccacgatggatgattagaaagatgagttgaggaattgaggaggattgaaagagagaggtgatcttagttttggactttccattgccaggcccggtaacacccagctgtattcatttttctgatctgatccccataaatggctgcggtattcttacaattcccttcttcttaggtgagctagtttgaatgagtttctctctctctctctctctctctctcttttttttttctttggaggaaaagaagatttggaccagaatacctatatttctcaaatacttgcctcttctctgtttctgtaaagctgcttcttttttttagaaagttgttattgttaaaatgatgtctgctatgaatctttcatctgaagtcaaacaatctgtaaattacatcttttgtgcactgagagtagggtgccttatgaatctggtagtcctcctgataagaatatttgaaattctctcactttattactgaaatttgaagaaaatccaagcaattatttttgtctctatacttgaagtccctagagttggtgaggaaataattcactcagattaatattctaagagttaagtgccgttaaggagtttagaattttgaattatgtagttataaaattatattaattataaaattataacaatattgttttaatttttttgccttaaaaattgataacatactgttttgagcaaattttaaaaatttatataaattgaagcatacaggggcccctgggtggctcagtagttgagcaacttgcctttggctcagggcgtgattctggggtcctgggattgagtcctacacctggctccctgcaggtagcctgcttctccctctgcctatgtttctgcctatgtctctgcctctctcttctctgtgtctcttatgaataaataaataaaatcttagggcagccccggtggcttagcggtttagccccaccttcagcccagggcctgatactggagaccctggatggagtcccatgtcaggctccgtgcatggagcctgcttctccctctgcctgtgtctctgcttctctctctctctctctctgtgtgtctctaataaataaataaaaatcttcaataaataaacaaataaaatcttttaaaaataataaaataaaataacttgaggcatacagagtgagagaatgcctgtgacatttatcttagtgaaaaaggaggagggggtataaaagtaacatagtgtactagctgatatttcagtccccctgggggattaagtttttcaaaataagagcagccagttgagaaatcagcatctattaagtgaactgtactctttccagtaaattgaaatacagttggcccttaaactatgtgtgcacttatacattttttttctgataaatacagtatagtactgcaaatgtattttctctttcatatgattttcttgacattttctttactcttagcttgtattgtgatcatatagtatataatacatatccaaactgtgttaatcaactgtttatgttattgagaaggcctctggtgtatagtaggcgttcggtagttaagtttttggagagtcacaagttagacacagtattttcgactgcatggaaggctggtgcctctagcccccaaattgttcagatgtcaactgtaccacctttgccatgtgatgaagcctcatatatcctggaatctgcattctgaattctttattctgctccactactgtgtttatttccaaaccaatatctgctagagaaagttatcccaagagattttcttttccataattttcttgcttattattaggtagtctttcattcttccacttcgatttagagactaacttatctacttctccttccatgaaacaatcggaatagtgtgtaacacttgatttaagggcatttaatgaaagcaacccaaccattttaaacaaaaagaaccttgatataggaaattaggtttttacataattgatgtaagaggtttgggagaactccttagtcgggccatcaggagtaattttccaaactaggctacaggaaccagttttcataactggccagggggaatctgctattttaccaccatcaggaagctggggaatcagaagccactgaaccagctgttggcacctggaacagactggtgccagaatcccaagaacttctggctcacgagccatctcctatttgctggatctgcaccaacaaaaataaatgcctttcattccatccaaatggtattatatttatacacttatttggggagaatcaactttaaatgctggtaagtcttctattggattaatttgagtagaaaattctgtgtgttaaaggtcctaggtttcagaagaaatatatagtaacttggtttatttaaatcaaatgacaaagttggaaactttccactcacttatgttataaccacatgagagttgaactttcctgatgtggttctgtgaacaaatgtaacaagatatggctttctatatttaaaatgtctttccatgtaaatgaattttctcacaaaatttattccttgaatcattaattgtgacctttatattgaaaatataggaaggaaaatatctaatattttattacttaatattaaactgtataaaatgctacaatgaaaaaaagcatgcaatgccttttaaaaagaaatttaagatagatctttggatatgcatatagaattacagtttaaaatatatcaagattttaattattattacagagtaaactaatttcctatctctcacctgttttcctatacatttttccaatttaacattgataaataatattttaaggacaactaagtgtgtccacatttgtggattgtctataccaatatctatataaatctatatcagcttaaatatgggaagtgtcacttacttattccttggctgagcaaatttctcatctaaaatctaatttcttatctagaatacataacatacttattttcaatataattatatgtgattctgtttgtgaagtgcctggagcattaatagtcggtgctcagtaaatgacaaatgttactactttcagcatgagatagtattcaaatttcacttttattagaaggaaggtgtttacatggctccttctctgtgcttctggtgtcatatacttaaatgaatgtccagatggagagcctcatataaatgttctttcagtttcctgtttaatttttctaattgctgtattaaacacagtaatatttcaaacatgtttctctggatattccctatagattgaacctgtataaacctataatatgctatgaaaacaaattatatgattctatatgtgaaattatgtgttgtcttcagggaaaatagttaatgggtaaagaacatttttctcccattttgaaaattctgttttcgtgttatattattttaaaataaaaaagtgattaatttttcatttctttactcttgaagatgatttatatattatccatatattatccttcatcccctctgccactccccacaaagaaaaaaacacacacacagaaactggatcacagtggagttgattaagcctcttaccttttgaaatgaagggatttctaataaaggtaaaaataatatttggtatagctgtataggtaaagctcaagtgatatattcggaattgcttgaattatgtctaagcagtttgttctccaaagagtttaaatcaaaagaataatagaaaggtcctaagaaaatttctagttctgagtaacctatgggtaattagaaagcattttttaaaaggtttcatttatttattcatgagacagagagaggtagagacagaggcagagggagaagtaggctccatgcaggagcctgttgtgggacttgatcccaaaacttggagatcacgtcctgagctgaaggcagatgctcaaccactgagccacctaggcatcccattagaaagcatttgtaattatggaaagaaagggggagcttttctatttcttttgtgaggaccagtgtgctgagaagttagcaattaacacacatttcatttctgcttctccttaggaaaatacagaagattctttaaaaagttggttcatattttattttattattttaaaatattttattactttatgactcgttagaataattttctcatctggtgcatataaaaatctaggctcctcactctttaggtaattctgttacaagatccatttgtacctcatgatgaaaagaggaaagaacatttggtaatacttgcagcatagtaggtggactgtgattgatagttatcatgtctgttgctggatgagtaaatgggtggccgcattggacattatgaagggtgactcacaacttcttttatctgatgggcaatacgtgaaaatcctgagctcattcttccagggcctgctttctttatattgtgctgctcaaactgtgttgtgtagcatgtatgttccttggaaaaagtgtgtaaaataagttttaaatgtgcatactgggacattacgaaggtctgagaaattctgcaactaacaatgcatcctcaattttctctaatccattgtttcacaaatgtatttaaccatggaatgcattgttatcttaccactggtagagaggaagtttttttttttgtttgttttgtttttttttttttttttttggcaccaaattttcacccctgtgaaatggcagatagttcttgagaggatagttctttcaaaccagagattggtaacactacttttagctagattttcttcagtgctctggccaccttttgaacaagagggagatgggcgcatattaggaatagaaacacaagaggatacacagagtagggaaaatgacattggtaggatgtgtagggcacatatgatttgcctatttctttaattcccttctgattaattctaatcattcaaaagagtgctttgattgtggaatgtgccagaaaaaaatgggaggaaaatcagaaggggattatctgttagagaaagtaaagttgtataaatgaaataaaactcagaatatgcatatggtattctaaatataaggatgttttggagtgtgtgtgcttatgcagttggtatgtcaatagctgtatgtggctcaactgagcaattgattttacagatattttcacatttcagcatttcagactatgtaaacctgccactatctcaataatcatgtaaactatgtaagtggtctcacattgtttgaatactgggattgtattcatcaaaatgttccagaatgaagatcaatttgaatacctggccataacccgaatgtttccatgttagaaatgtttgtcagtaagacacatctttgaagcaaataggttattaggagtcacccaaataaattaccgctgacatattttcctaatggaaacaacagtaatttatataaatgcaaagagtttaaactgacaattttatgtcttggagattgacaaacagtgcatgtaaacaacagtaataagacaatgaatctattcattactcaaatagattggatgctctttaaagggaggagtagcttctggcatccatttagcagaggtaagcacatgaatagatgcttgctgatttgctggtagcacaagaatgacggtaaggtagtcaaattaagtttatgaaaaaggataaacgaagttgtagccatttctgaatgccctatttctaaaatgtaaatctaaccttacataatgaccctggttttggttttctttttcaaattgtttggtttgcagaagcactttctttaattatgaaaaaaatattaaaaattcacgacaccttttacacaaagataattttagggcattttccaatattaagttaatttttttaatttgataacaactagtttagttatttttactatagccttttaatataacttagaatatttggcttattaaaatatcttctggagcaaatagaaaaaataggattaggatctaaaagcaaatgaaatttaatattctccatgtgtgtctagaatggaataattttgttttattattctttttttctagaataataaatgcacataattgaaatatgtacagcaactaaaaatgtaggaaaaaacatggtatatgttcttctagcttttatttttctatgttgacttttaacaattttacttgttcagctttgcagtgttaatatatttaatatttgtgtatgcactttaaagttacatttgtatggctttggtgacttgtcattcataagtatttcaattttgctatctcctcatgtaaatctattgtacagtgtttaaaaacaaaagaaaaaggaaaatccacaatgatttattgggtaaagactacatcatcttccatataaaactattattaatctaaagaaagactgaatagttttttgttgtttaaattttttaaaagattttatttttatttattcatgagagacagagagagagatagaggcatagacaaagggagaaggctctcaaagggagcctgatgcaggactggatcccgggacccagggtcatgccctgagccaaaggcagatgctcaaccactgagccaaccaggcatcccaagactaaatagtctttaataatataggaaatactttttcattgccctggatttgaagtaaaaaaagcacaggattaagtaatggcttaagtaatggctctgtcactaatgagccacgtgacctctctgatgcccagaagacattatgaatattgatgatcacattaattactcatgagaatgattggctcataataataatccaattactatatcttattttaagagaaaaacattgcagaaattaaaattacttcttctaggtatctgggattgaggctatccatcaagaaagagtggaccaattactctattgtcttatgcatgagtaattatcctgtattcatttactcttcaaacatttattcttgatctgtgctaggttcaaaaaatacataaaaaggtagctccagccctcagggactgtattttagagaagtaaggatgggaaaagctgagtggcattatttcttatctacactctaactgaacattagatgactcaaaaaccttcattgctgaaatcttaataatttggttatgagaactagttagccgggtgagctacagctaagagtgtttactgtgactactgaagataactcctgatgcataacttctgttccagaaagtcactacttatttcagaatgcagtcatgagtacagcccccagggacagagttttgcaaatttcagtcgttctcacaaaaccttcctcaatttttggttatagtaccctcttgtttaagaaaaaaattttctttaatttttttagcttaaatatattttaaaggaacatttttatcactaccgtaaatgaaaactcattaccactcgccataaagagaaggtgaccatacaaataaacatattgaacaaaacaaaacaaataaacagattggaaataaaataatgtttaaaaattataggtagggacaggttttgggcacataggtcaacctcaatttgatagtgttagagttcatgaaattgcaagagcccccaatctgagacactgtccttgactgaatcaggattgaagaagtattctaaagggaatgactctctatgattccctgtcactatgtagggaattggcatccctacctactggcaaatgctatccttcaaaggccaatttcgccttattgaaatggttgaaatgaagttttagttaggacttaaaggtgtcaggaatgatgataaattcttcacatagtttatctcatttaattctcacaacaacagatttcataagtggtattatctatatgttgcaagtgaggaatccaagctcagagaggttaattattcgcttgaatctggctgaatttagattccaagacagcctgactccaaagcctgatctcccagccaatatattaactctctgaaatgaactcattttattctcacacaagcacatgcgtacactcagaaaagcattccctcccatgtggatatttccatttttatagtcaatgaagtagccgtgtatatataaaaagtcattttaacagaaaatgagtaaattatattgttactcattaataggtcaacatcgaaacattctttacaagggataaaataagcaaccactgtttctaactaataattgtaaactttagaaagttttgctcttttcaagacccttagagttgaatgaagtaacaagaagtgaaacatctatggcataatgttagaaaaacaatgtatttaatgaaggctgttaggatatttaaaatcatcaaggctgtaatatagatgtgattaaaatatattgcaaaatggctgcaatgaagagtactcaaaaatgcaagcctaatagtttgcatttgcctaatgctttgcatcttcaaaggaattttcaaacccctttaatcgactcagaaaatttctaatgtatcatttgctttgactcagtgagaaatctaaaaaacgatcatttatttaatcaaggtcatcctgcaaacactctgtgaggaagcagtaagtgtacttgcacatatatctattttacttcagaaggctttaggaaaggaagaaaagaacctagagctgaaaatagtgttcagatgtttgatagaatactagctccccaaaggtgtccacatcttgatctctggagttatgaatatgttaggttacatggcaaagagaaattaaggttgcagatggaattaagattgctaatcagtttaccttgagatggggaggttatcatggattatccaggtgggcccagtgtaatcacaaaggtccttataagtgaaagcagaagagagaaaccaggggcatagtagcatgagaaggccttagcccaattttgctggcttgaagatggaggaatggggccaagagccaggatctgtggatagcttttagaaactggaaaataaaaataaatggattctttcctaaggtctccaaggaatatagtgctgccaacatcttgattttagcctagtgagactggtttcaggcttctaaccttcaaaactgtcagataataaatctgttgctttatgccgcaagtttgtggtgattttatatagcagcaataggaaatgaatgcagatttttaaacacatgtggaccccacaaggacgaagagtagcatgcataccacagatcaggctgtttgcattagacttcacataccattggtttagtgaaagcattggatgaaaccatgaggaaataagcttttataaaatataaacagcatatcaatattatccttaggtggttatctttgacaagatatatccttactcattattgaaacatctctctggaattcactatttttatagtgaaataaaaaaaaggagtcctgttattgttcactttgggtgtttttgccagtttaataatttttatcattactgctactggctgctgttgctattacaattcctactagtacagacatgtgagcttcggcaaagtatttactttctcagtgccgtaggcaccacaagggcaaaatgggatatgaagagcacttgttcacagggttgtcatgaggttaaaagaagttcattcttgtaaaacaccagaatagtcacaggcacatattatgcactagctccatttaggtagcctttattataattactatcataaaaaaattatcatcaccactgtcataattaccactatgcttagcagtgactgctgaaggccaggacctattcagattttatctctaggaattaggagtcagtctctaccgtttctcaggttaagaaactgaaacaacttaaccaggttgaggagtgtggctgtcttgttatatttggctaaacttcttgtagggttagtgtgctagatgctgtcagcttccaatacatagtcttttccatcccaaccctttgaaggacagagatactctgagaggcaactgatgtagatgtgcctccaatggtcactgatattattttatggtcacactttgtttaatatctgaatatgaaccattttgaatcctagtagagatttattttgagattattttctcctaatgataaatggttggagtcaggctggttagccatgaataaaaccatatgtgataatttctaagggtattttcttataaaagtaatgtatgtgcattgtaaaacatttgaaaaatagagaataatataagaataaaattatagtatactataatacacagtcttcccctcagaaattagtattgataatatttatcttgaattatatgagtttgtgtgcattcataaaagagctcacgtagtatatatggtcttgtaatgttcactgtgatttgcacattaatttatgatatattttcactcttttatgtcgaaagagacattttgtaagatttaaatcttttaaatgtttaaataattcaattttgcaatttgtaatattgaagaattattggtactttcttttcaagataatacttttacattaaacctaggataaaaaaagttagaagatttctcaacatggatcttaatgcaatattataataaagtaccttgtgttgaggtaatttctaaaaatccatttccttcttataatatgtaagttcctctaaggcaggaattgtgcttcataatctttatatctattaaggtctgaacatttgttttgaataataatcattaagtgattattaagtaaatatatgaaaagatataaatgattatgaaattgatatggaggtattgtcaacattttgataattatggatgattatggaaagtctcttgcataaatgtcaaaatgtttgatctgacaatctaagcaatgaatgagactatcaaatgttggaacagtttaagtagatgatggggtttaaggagtgcacttgtaatgatgagcacaggtgacatatggaactgttgaatcactatattgtacacttcaaactaatataacactgtatgttaacaaaatggagttcaaataaaacattaaaaaagaaattaaaccagaaatagtgttacagtaatgagggtcaagatcaagaaagggacagttatgtgttaaataaggctgtctggacatattaataatttccaaagaatgaattgtatcatttgtctctctatatattttaggtctgtctgattttcaactttgcttgtgattataaactatgttaaaatataagaattctttaagttcaagcagtaaaatttttaaatacttatagtatcttatttttttgtgttcagaaaaaatataaaggaaaattcagtagttggatagctatttctctacactgtgtttacatgtaagtaattaagcaataagattcaataaataaaacagattactaaaattgagaataaatgtaaaacatttactttaaaaatccttgttagctctatctctaaaactttatatggtatagagcttctccttatatattttaataagagagaagaaatataaaaacagttgtaaaaattctttgtttcttcatactcaaaaatcctatttctttgtcagaaagttcacatttttaaaagaattcatgcacagattttattatattatacatttccaaatgtcaaatatgaatgaaaatagtcccatcaggaatttcctgtgcttcattacttcagttcagaatagaattcagtatttaatcttgtgcaaaatatcagccatgggtaaagtaatttaacattaatttttttaactttggcttttgtaaccaggttaaagcctgacttctgggctgttagttttcctaaaatggcacctggagactggtaattaataatagtaaaacaacaatttttgtaaaagtctgaaaaaaaaagttagtatgaaatggaatacttttattgttattcttattattattttagattttatttatttattcatgagagacatagagacacagagagggagaagcaggctcctcatagggagcccaacatgggacttgatccctggacccggaatcatgctcggagccgaaggcagatgctcaactgctgagctacccaggtgtcctggaaatagaatacttttaaaattgcttttacatgaccacatgtgctataaatccccacccttcaccatctatgttacattatacagtttggataaatatcataaaccaatattcactttagaattaccattttagctacataatttcaaatatcgtgttttttgtctgaatacaggcagtttggccatatatgacagacaaaatcatattggtgaaataaggcagattttaagcaaatgatttaatgattttagtgaattaaattatacatatgaattcattccttaataaacaacataagccatatggattttttctcattagaagaaactaaaatctttatatttttataaatcagcaaaacttgagagataataattgtaactgaaatcaccaaatatgggcagccccggtggctcaggtgtttagcgccacctgcagcccgggtgtgatcctggagaccctggattgagccccatgtcaggctccctgcgtgaagcctgcttctccctctgcctgtgtctctgcctctgtctctctcctctctgtgtattctcatgaataaataaataaaatctttaaaaaaaatgaaatcaccaaatatggtactagaaatactatctatatttaaaaggattacattaattctcacttgaatggactagagaattaccaggattgctttggatgcttcattatatgatggatgataaccctaatcccaattctttccaagttcccaaagtggttgtggtgcaagtaataccttgtccacattatgagaagagaatggagtcagaatggggcagggtagctcttaccaaatgcacaaaagggttgaggcctttcctagaggcacccagggttttgattcattgccattgtgtgagctctacccagaagccatgtttattgagagcataggccccaggaggccttgggttgcttctgggagtgataggagatacactttgctaggtctgctgtttcccttttagtcaataatggctgcagagaacgcagatatcaggaaggaaggggtgggagtttacacgcttgctggtgttactttaaacaaatgagtagacaatcatcctattcttcctttcagtatgcatatatttctagtatatagccatttctaagatattttagactctactatattagaatattttagaaactactatagtagtttgtgagataaacttcctgcagagttttgctaaaggtgtgttcttactcagttattttctcatattatgacatcatatgtaattctgcacataacaaaactattgaaagacattagtgtctaaatgataatcaaatatttgcttatgtaattagattgcaatatttcattagaatttgaggacatggagtgtatttgtcatgtccttgatttatatacctcagtaatggagactgaagagtatttatttgcatagttaaatctctcctaaagtccatgaaaggaattcaggaaaaaaaccaaactatctggtcattgttcttaagccatgattagctgtactttttcccccaccactgccatcatcttgtgctctgggaagaaggggtagatatggggagtgagggagagcaaagaaacaagggaataccaacacaattttctcttgctcagaactagggcagcaaaattagatttgccaatgtacctaaagataaatctctttttaatcggatcaactgatcatatgctcttatccattgggaaccaacctcaatgaagggactgaagtttaattcttttttttttttttttttttttttttgagaatagtgcctgcattgtggctggatactcaagccagagttggtagtcagtgtgaatttaagcccagctctgtgacttactcttgatatgatcttgggcaagtctgtcaaacattccaacttgtgttttaagtttaaatttttgtttttttttttttttaaatttttttttaaatttttttttttttatttatttatttatgatagtgagagagagagagagaggcagagacacaggcagagggagaagcaggctccatgcagggagcccgacgtgggattcgatcccgggtctccaggatcgcgccctgggccaaaggcaggcgccaaaccactgcgccacccagggatccctaaatttttgtttttaactgtaattgagttcaaaaccttttcttaaaactgcaacactgtgaataaagaaaatacaccacaaaattaatatgaatgcatttcgtgtattattagtattaaaaggaacttaagttcttttgaaatatgtcacatttacaattcagtgttaagtcatcttccaataaaagaagagtcagagatttgtattgagaatggtgatgggatggaacaataaatatccctcttcaatcctcaagttgttaaacaattgagaaataaaacaaaagaataaaatagaaatgaaataaaatattgccttaattattgatgagattggcattgcctactgtggtgtatatctgtgagtttgtaccatgaaatttcagaattagacagagttacacagccaaacacagtagatctttgaccatagaccaagagctatcaagaacatatgcccttgtatactacagaagcttaatgaaacaactacaggtaaggattgggatactagttgaattctggtccctactaatgtctcctggtcatttaaagaagatgcaacttttagaatagagatttactgaaattattagtgactactttagagttgtatcatgttctctcttcatatattttcaaggttaggtaatatggcatataataaatagagtttaaaatatattttaaatagtttagttactttgttagggagttgccattctggacatattttttcaatttttgttaattccttgccaaatttatcttttatagttatagcaattgaggtacaatcatgacaagtgcactcctaaaaccccaacacttattgccccatcccttacccaccttgtagtccccaataatttgttctctgtagttcagattctgttacttggtttgcctctcttttattttcccatttgctcatttgttttatttcttaaattctacatttaggtatgagtaaagtcatatgggtatttgtctttctctgactttcacttagcattgtaacttgtagctcacttagcatttcacttagcattgtaacttttagctccatccatgttgttgcaaatggctagatttcattcttttttatggctgaataatatttcattgtgtatggataacacattttatttgtctattcatcagtcgctcaacacttggcctgcttccataatttgaccattgtaaataatgctgcaataaacataggggttcatgtattcctttgaattagtgccttgtgttttggaggtaaatacactggattttggagtagttctatttttaactttttgaagagcctccatatgctttccacagtgactgcacctgtttgtacacctgccaagagtgtacagggttactttttatccacgtccttgccaacacttgttgttttttgtgtctttgattttagctattctgacagatgtgaggtgatatctcattgtagttttgacttgcatttccctgatgatcaatgatgttgagcatccttctttttttttaaagattttatttattcatgaaagagagagagagagagagagagagagagaggcagagacacaggcagagggagaagcaggttccatgcagggagcccgatgtgggactcgatcccgggactccaggaccacgccctgggcgttagcaggtgctaaactgctgagccacccagggatccccctgttgagcatcttttcatgtgtctattggccatctatatgtattcttgatattttcttaattagtaacctcatttgtatattttaactctgaaatgagcatatcctttcctgctctttcttcttttactgatagtagatttatagttcgggtagacaattaagagactcaactcaaagttctgtcaaactgggagaaattcttctcactgttctctttcagggatagctatggatgggctatgatgcagtaaccactcattctgggctgttgccagtatgttttcagacccattcaaactgctgtcatgcttctttttttctttcacaagttaaataagtctccatgaggctatagctatgtattgagggaaaggtgttaaaacaataggatctgttgtcacagaagatttagccacatgctcatggaacttaacccatacagacctacatggggctggttgtgtatttatcatttcggtttcctaatagaatatgattactcacaatcaaatgtatattttggtaaagaatcattggaagaataaaacatggtctctctccaaaactttaggggtatgaattatggattttttatagttatcagaagcatgaaataatattattataaagtccaaatgacagggaagactgcactgcagccaagccttatagcagaactctttcttaatctcgccccacttagagctggtatagttattggttaactggtaagtggtcgggatactgacccagatatatatgcttccttcaggatacaaaaagacctgtccagtggtaaaattcagtaacttatttttcaatatttaggggtgtcctgatatgccttaaaccattgggtgtacagaagtttgcaatatttaaggacactgaacttcagcatggtgataaacctcacactctggtacataagtgtcatatattaaatcttctaggggatttgctctttgtgagcaagtttaattaaaataatatcatcaattatacattacatatgtattacaatatgccttatggtatatcaatatttctgcagactatattatgacagaaagcatagccttgaggtaaatcaatgacaggtgctcctgtccctgccaggtgaatgcaaactggttattatctttattgattgccgttacaaggaaagtatttaccaggtcaatgtctgtaaataatttctgtaagctggttaatttgccccaataaagatatgtgtagaacagcagttgatatcaacacctagtaatgttgatgaaaattgacagtttgtaccagtaagctgatgagttgagtgaagatgtgaagatataaccactattacatctttcaaatctttcttggtggtgccaatccttacaattatcccaggaatatagtatttgttttaatttatgcttttggtagtgggtggtgggtggatgaagctgaacaattctagatatttatatattccctttcctaccataatatcttcattccctgtaagaattctgccctttgctatgtatttatatttccactatatattgcccaggatatctttatggacccattggaactattattaaatatgcccaaggcaagatgtcatttgtcattacaccaggtttccataagttcccattcttaccaggatgccattttaggtcttcaggaatttgtgtccatggaaatcaataaataataaggactaggagatatgggtatacctttttcccagtgcacatggctgcaggtctttggggaaatgcttaaaggaagattcattgtatgcatgtttagaaggaaagaagaatcctttctcaagggatttggccttccctcagtcaaagggttctgagtctgtaaaccaaataggaggctgtgaatcttcacttaccacagcttaagtcattttttgtacccaaatttagaattgttttgtttataaaggtaaaactgaagcttcatctactatacgtatagtttatttataaggactttcagatcagttacctcaacagatccttgcggtgaaaaatatgtggattacttaattgcctctaatgattaagtgctgtcattaattctctaccaatccagacttcattattcctcattgatatgaaaaagttcgagacccctgagtggctcagtgggatggacccccaagtctggctccctgtttggcggggagtctgcttctccccctgccccttcccccacgcatgctctttctctcactcactctctgtctctaataaataaataaaatatatttcttaaaaagttcaatttgaatatccaattcttattatcatgtccaggctacacaggatagataccacagagtttttcatgattgaattttttgatgtttagtaaagagaatatcagaatactctagaacaagggttagaaacctgtaaccaataggcaaaacccaggtggtgacctatttgtctatagtttatgagctaagaatagtttttatacttgtaaaaggttataaaaaaagaaaaataagaaggaaaagaggtggagcagtggggtagagagagaaagaaaaagaatatatgtagccccaaagcctaaggtatttactgtctagctgtttaagaagaagtttgcctgcctgccccttttcttcataatggaaggcaagtctggcaatgtaatcacattactctatactgatggagtccagattttaattatccattctaataggctgctagtgctaccccaggcacttgagttctgaatcttataggaatacaaccatattgatggattcattcaggtttagtctttctcttttgccatccttggtctcatacctcttaaaatccattccatacatagtttccagactaatgctgatacatattggcaagattctgtaataatttttgcatgtgaaatagtttttctggaattccttttacttatgggatctcatgctagttactgatcttatgggcaatcaaggaggagtgttggcatagatgctgaggaaatctgacattgcttgtaaggcatttgtcttaggtaaaattcttaaaatgttttctttttaattttgtttttgtttttacaagggaacacaggttccctcagttaagaatgattcagagatgcttgggaatttaaagtttgggcagcccaggtggctcagcggtttagcaccgccttcagcccagggcctgatcctggagacctgggatcgagtcccacatcaggctccctgcatggaacctgcttctccctctgcccgtgtctctgccctcgcccccatctctcatgaataaataaataaaatctttaaaaaaaaaaaaagttctcagcctcatctctgtccccagtcttacttgagtcttatgattctaccctttcttaaactgtcctcacattggtttaagaatcttggaggggtctccctgctaccggagctctacccctgtctttgttcatatgcatgctctctaggacagtcagagaagccattatatatccttcgcccttctccataatccctattgccaccgtattaactaaattccataggcatttgatctcctaaaactttcccttccattgacagtccataccaaattatccctggtgaaacactgagtcattatgttgcttctgcatgccataaattgccaatctgttattttcctttggcaaggggttgtttatacactctgcaattatgaaagcaacacaattccaaaatccatctcatagaatttatttcctgtggccactcccagtacctgtatttattagaatcatgacaggaaacagatgtcactatcaaagattttaactgaaaacaattataaaaggattatttattaacatgtgcagggctgaagaactcaagcagggatagttaagtattggaagactaatagcaggaaccctttactacccttgtgagggaagaggagggacaattgttgctagagcttatgaaaactggagccatgaaagaaaagccacagaaaagaggccacaaaagaatgcagatcctaccagaactagaagggggaagtgagtgagagagccagtaatgaactctatcattcctttttctcaccatctagtgttctgatggtgctttccactggcatgggcatgggagcctaggtgatgtaatcagtctggacagatcattaaaaatatccaagtcctaaggagattattcttgagagtgaattgaagaaatgagaagcacttgagaatatgtaagatgatactattaccatatttaatgaattctgaacgtgcttttgtttggagctagtcaaattctctgaaatcagaattcatcttaaaatttatagtatattggggttcctgagtggcttagccagttaagcatttgactcttggtttcagctcagatcatgatctcaggattgtgagatcaagctgagacctgcattgggcttggtgctaggcatggaggctccttaagattctctctccccctctccctctgcacctctctcctccctctcttaaaaaaattatagtatattatgtttatttgatcacagtattttttcttagaggaatatcaaataatagcctgtcttatggtcagtgttgtgttagattgagtaatctatagagtaatgcaataataggagggaaaggttatggactttgcagaaataggattccacaaatttacactaagtaagttttaaatattttaaaatctgttcatagatatacaatcttttgtgtttataattttcaaataaactattaacaatttgtgtcagagattaaaaactgatagaaatagaggaaatgaacacctgcagtttatatgtgaggggtagaagggctttgcaaaataatcctgtaacagtacaaaccatgcttttttttacaaaaaagaaaaaattctcatgcaagcttgttctgtgactcataaaagcatttgtttgcatctttaaaggaagaagaaagaggaaggttttgcatttgtataaaaataacttttcctggttgttcttcattatagctgaaaagcataatcttaaaatgatgtgttggtgccttcctgtatttgaaaggaagagctaagttatatatatatatatatatatatatatatatatatatatatatagagagagagagagagagagagagatatttcccctgaagaaaactgagaactggacatgagtgcaggtagttgatttagagaataagcccatgaagcactagtgaaaaaatagggaatgtgagacaaggggagaaaagcttaataatgtgtatattaataagcagaataacaatagacaattacaattcagcatcactggggttcctctaattactgtatagaattaaaccccagaatagtcctagtgaaggaagagctgttatttacacattgatttccatcctgcattgactgagggttgccttccagccctccctgccctaaatccccacacctctgagctgccaggcagatgcacacactgaggaaactccctcagagcacagaaagtgttcaggcagtagaacagaggttggaaatggtcattgtgcttaatagcctatcactgagctgcagctcttgggccaggaagatatgggatgtacattagtagaatctgctatacagatatataaagaaagcacctgatattttttgctactatgaataagtgaaattaaccaactttcctggggaagaaacatgagttatagtgtgttttactacattaatgcctttgaaaaaaagtccctagttttgtttctttgttgtactagttacagcacaaagttagaggataagacaagtttgtttacatttactgtatattttgaataatgatatattttgtgtgtgtgtgtgattccatcaagattagtgttcatgttatgactactgagtccattatggctataaataatttcttcgtaattttctgctctttttttcctacaatttggtcaccaggtaaaaaaatataatattgaaaagatgcattatactgtttttcttcattatgcacacatggataaaagatgggatattattcaaaaagaagaaaatatgccatttaaaactacatatatgtaataggaaggattttgattcaggaaggattttgattcatgaccagtcacatttcttttggtaatatgtagtgtaatctgctctctgtgtaaaagcaattagtgatatcaattactgatacaaatttccttcaagttaaaaaagttactatagtattttgtaggataaataaaaggcatcattatggaatctgaaagaacattttggagtatatttataaaaattacatggctgtattctgttctatcattgtaaaatgatgtcatttacattatgttgccagactggaaatttacattttaaataatttatttacagactttatttaatcttcaaaagcaatttccttttattttgactaagctttttaggaatgaaaattttgtatttgtgtatttcagattcatcatttattatttcatttcacaacctatagaagtcagaaaatcttaatgacaataactcacattaaactggcaaataatgattaagtttgattatttcaagtgtgtaaaacaaaggctagtaggtttgtcttgagagtcaatagagaactattgttggggttatagtccaaatcttactcaaggacaagcttctgttttgtagagtaaagaagagaaaaatcatggctgtgaaaagtgcttttattatcttttgtattttgactcaacatggtttcaaaaagaaaagattggaaggcagcataagaaccaattactgtctttgtggccctgacaagttacggaagctttttgaggctcagtatgatcacctgtaaaataagaataacaagacaactgcatatagttttatgaatattaaatgaaagaatacatgttatgtgcttagcaacacatattattaaataataattgagaaagttaaaggttaaaatattgaaatgattttatccatagagaagaaatggatgacatttaatgcttttgaaatgtgtcctgtaggggttattgactaactataggtcaaatataaatttaaaaggaacacttaatttcagcctaaaagtaaaatggccgtagagtattgagagagactacctgtctactctttcttgggaagttttgaaagataggttattattgtctctagatttgatatcttatggtagtgtgatacccaaccacataacatgggaattactcattttgtactttaaagtttttatttattttattttattttatttattttattattattatttttatttattttattttatttatttattattttatttttagagagagagagtaagagggggagaggagcagaagaagagggacaaacagattccacactgagcagggagccatacctggggtttaatctcattaccctgagccaaaatcaagtcagatgcttaacgaaatgagctacccaggttcccctcattctgtactttcaaatatcctcaaagcttgttctgtaattttattgatttaagttttagagttaagaagaaagttctgtaagttcttcatagataaccatatctggagtatatcatatgttctatttcctctttttccttggtaggaaaccagagtcccggtaagctttcacatactttgcatacttaagaataactcataggctgctaaatattgactaacctaacttatatcactacaaagaatctatcttaatctttcaaataattctatagtgtcctttgctgttagaggttaaaactataggctgtaggatcagactgcctgcattggaatcctaattcccgtaccacttcagtttcccgtctgtataatgacattaatatttgaacctattacataggataattttgagaataaagtaaaatgatatatgtaaaacacgtagagtcctagtttacttgaagcatctggtaatttaattattagttttttctttactgtccttattttataactataaagtagcttactctagctgtatataataatagaaggaaaactttctctactgttcctcaaacataaaaatactgaattacggtaccatgtttgtatttgttcaacatgtatttatttagcatccaggctgggtgagttactacacagggaataacagaactaccttgatgactcatacacaacttgtgtatgatttcccatatgctttctgccagatgtcattttcatcctacacacataaataaattttaaaaatctattgaaaacctgttcaagaaattgtggtaggtattgtttaggaatcaaaaagttgaaagagaaagcatctttcttctttcttttgatactttactttttattgcagaataatttacacacaaaatctaaggtacacagataattaaatttttatctgtgtatactttatgtgacagccattccgaccaagataagaacatttccagaatcctggcaggttctcccatccctgctttcatttaatcctatgcccagatactctatctgcattagcatggatcttttttctgtttttgaacttcatataaatacaatcatgaaatatactttttttttctttcacctcatattatgtctttggatttacccatgtggtacttaacagtgattcattttcactgctgtataatatactacaatatgaatacactataaaaattatttttgtaagaactttcttataattcattataatctgggtttgaaattgctctgaagaaatctatggctaacttaatatttttctcctcctgggtgacttgctcttccttcctgagtaggtggagacattttataataatcactaaaccaaacgcaatttaatcaaaataggttttcccttaacaatttttcttagaacatgatcgtgctccactgatacatgaaactggtttctttttatttcatattttaaggacttctaaaattacatttaaaatattttgctatgtcttttgttctactttagcttttaggttgtcttcatattgagtcttttcctatttttagtcttttagccagatattcatatttttaccaattgctaggactctgttctctttttctgcatttactacgaaaatattgagcttttcttttactccagaaattaaaattttatctatgtttaacctagttattgcttttctaatttatatattatcagggtaatggtactgtttggctcaacttgtctccctatctcgtatatacttttaaatgatatttttgaaaaagttaaatcaatttctttgaactctgattttaatctctggtctttttatactttatactatgggttttttttcttctctgcttattatttctactagtttttgcatgctgtgttttatttttatgatcatattattactgttaatattatttgcataatatcttctcacaacaacaatctaatctgctaatcttttcatcttgcttgtgtttctcttagctctacttaggctttctgtttgcttaatatagtgttgataaattattcgttaaattcccctccctatccctatcttttttgcttttaatttttccctaaagctacaacttgagtgacaaaatttgtattccattcaattttttatgacctgaaagaaaaggagaggagatgcaaagtgtcctcggttggaggtcctgggttgtggcctctgagacctgttaaatgttgtctgcaaggccccttctcttcttgcccacttgagttcctgtgggatttgcctggttttcctggctcagcctgagtcctggcttggggagagtcctgacagtctttcctagtcctgcagacaccccctgatcagtttccagatgggaaagggattcataagcaccctagtggttatggtgatgactgaagtttctttttcggaattggttgtggggtcagagagaggtctgggattagtgctgtcctatcagaacctttgaattatttcatcagcatgatgacataagatagtaccctttttatttggtttctgctattgaatttttcttttttaaaatttttattttgttagtattctgggcatagaaattctatcacttgtttttgtgcactatctttacctggaaaaaagatttgaaagtttttttttatgaaaattctctcatgtctttacagaataatctgattccttaggtagagcttatgtatttacttttaccttcatttagtagaaggagaaacaatagcttaaaggggttaaatattgtgatcaaggtaaattttgtggttactggcacagccatgcctggtagcctagctcctgtaatcctacaaaatatcctttttattctgtcatgaaacttctctgttctttgggttgagcatcttggttcactctggttcaatgagtacagcgtagactcacacaaatcatgaactgtgttgaccttaaggtttactttaatcctgtattatttcataatggggataacatgaattatattattggtctctggtataataaaagtaatatattctttagaggatacatacactttttgagattaataatataatttgctgttagcacttcaaacataagaaaattaatacattgttttctttttttccagaaaatgcaaaggatatcataatgatatatgaagaagatgctgaggaatgggctctgtacttgaaagaagtttttttacatgttgtgaaaagagaagcaatcctattatatcacttggagaattactctttttgggatttggaattgctgagtttaaactgttataaatgtaaacttttgatattatcaaatggcctgcttaaaaacctaactccaaagaaatgtcaatttctggaaaagatacttcattcaccagaaagtgtggttactctgctgtgtggagtgaagagttcagatcagctctacaaattattaaatatcgctggaggcagatgggagatatctactgagcaggagccccaagattacatcgctgtaatcgagagtatcgtattcagaggtagtgttaccaaatcttctttatatatttttctataattaagacatctaaagtgtttttggaccaatgaatatctgaagttaggataaaagaaaataaaagccaacattatgttgtatgtatgttgcactttcctcaaatgcatgaaggttcttaagttttagctatgttccaatcagggcttattagttgctctaaaaaatggtttgtaaatatattccttagggtagtcttgtaagctcatatcatacttggtagaaaaaatatggtatactatatgtcagtttgattctagagttttcagaagctggtttcttacttaactgggaaggcttgagttaatatctaccctagtactgttaccagtcggtagcagtctaagctttgaatgtctgctttagaatgacaaaacttctgttgtgctgattattgtatggtatctattactacggtagggaatactgttatttttattagttttaaatttaaagaaaatttgttttagtcacatagctacatgttaaagagcagaacataaggttcttctacagtaattaattgcagttccatgtaagagtcaaaactctgaagctgtctattgttagtttctccctcctactgttctattctaattcctcctgggtgtggaagtggtgctgtgttagctgcattactggttttctcaacctgcatttagccttttattaccagtattctagcaatagctccctcttctacagggcagaggtgaggttctgattttctattggccatgtcaaatattaagtgttgcctcttctccttggagcatatttctgggttctttgacattccctctcttcactgggatgtgtcacctgtggctccttgatgagatgactgtgttctctcttgatgctcctacttgtggtggttagtggtttcttactgttgatatctacgttactcttctgtgtcacttttgatagaaatcactccgagattccccccagctggctttttctcccactcggcctacaccagtctgtaaaaacacgtgtttatcttttccttgtcaaactctgggcatgcaagccattttcagtatagccaacactgtcctacccttagagtagcaacatggccacagccttttacctttagtcatttccaaacatgggtctgcatgaatcctgttctgtagaaccaaagaacgtatgtcaatcacagatatttttgctggaggcccctgtcattgtgcttgaagatggtgccatctgtcagttttccccatggcgcatggtgggacacacagtgcaccaatggctcttaaaaaaatcctcttcctattttctagcattctaacctcttcaaattcagtatgttttaagccttgttttatagttatctattgttgcatgacaagttatccttaaatttagcaaatggaaacagcaaatgataattctattacagtttctacaattaaggaatctaggagttgcttagctgggaggttgtgcataagggtgtcttgtgaggttgcagtcaaagtgtccatagctacagtcatctgaaggtctcactgtccactgggggaaaatttactttcaacaggagctttcattgttggcaagtttgttattatttttttagttgtatcattggatgaaagtcatctcttatagaatcctgaggaaaggcccttgggagcaacatctgaaattttgcatattcaaaattcttgcgtgtttgtttcttgaagtatagctgggctgagaaccattttgctcttatttcttctagcattgtagaatatagggaattcttatttacctggctgataaatgattcttaatttatatttgaagtctagtgactttcactagaattttattctagtgctgactcttctgaactatttttcccatggacatgattatttttgtttttgttttgttttggtctttcccccaatatagagatttaagtgttctttacttatatctttaaatattcacactattttatcattattatttgcactattttattattattttctttttcagggattccttctatatgtatgttgaatttattttgtctgtgctgcactctataacttctaattctttttcattcatcattaatatatatttagcatgcatatatcacttcaacccttgctaccttcttatgctttcagtactttctcttcccctatattacttccaatttctccttcatttttatggtgcttttactttcttccattatgccatttggttgagcactgaaagcttacaatttgtctcctaatcttatactgttaaccttgccttgacaccttgcatttttgcattatatagttgcttcaaaaaggtggttgttctcattatatttgaaaaagtcgtggtgaaatatttggccacaattttccgtttctttcagggtggcgttttttttagtgtgtatgtcattggttttctgctcccctcctcccctgtccttttctcctactcttgtagtgtcttcatatagatccagtgctggtccagcttttttattgtgtcaccaaactgaacgagtttgtcttggtctagccaaaggtgatagccaaaatccgaacatgggattttgttgttggttttgttccattcctctcgcacacaatgaaagactttacttctctgtttccacagatagagactgctttctacaaatctgtcttttctgtgtagttccttatttagcctcactttctccgctttctgaaactgtactgcttccacagatgcttctacctgaaactactgcattatgtctgcaacataaaaaggcatgactcttgaccttaaggaaggtatttttgtttgctctttttgaaatctgcagcttctggattatttcttcaaggtttcttttctctgtttcctcctgcacacccttttgctcaatcatcactgattttggagccattccattgatctggaggtcttaagatttcactgtttcctgttttcaatacaaaaggcatttgcacaattgtcattttctatgttttggaacgatttctagaaacaaaacggggaaaatattttcactacagttttcaaattggcagtcctcaagtttcttatgatctctgggtttttagcactcttcaagtatattatagggtttgggtttttttttttttggatgaaaaaggaaaatatcacagttgggaattatggaaaaaattacaataatgtacatgaagtgcaagtgattttatgctaatttttctcccctgagacagaagaacattaaaaattggtgacaaaattttgagtgattgtccacactcatcatttctatttccttgaagaactcaaatttgttttgatcacttaaattacagtatcactggtaggtaatataaaaatattgagcaatatcttcttgctgttttacttattttgttttatgatgagtccccttgggtggctatggcagtggtgttctgctatatcttcaaattatatttctgttgatatttggtaggagaaaagcttacactttggataacataggactactttgagatacttaactctcatcttcagtccattcaagttttagactctgatttcttggaataacttagatgcacctgctaaatggaaaatacaacagagttgtgtgggaaaatacatttcaagatgatatatttgtctgaactacaagcatgcaatcagaatttagttctataaatgatttttaaaccatgtataaaagaacttttatgtgtaggttgtgttctttgataaaattggtgtaaatgctgtagtttatgcaataataaatttgttttaataaacagtttaaagggcagtcctggtggtttggcggtttagcaccgccttcagcccagggcgtgatcttggagacccgggatagatcaagtcccacgtctggttccttgcatggagcctgcttctccctctgcctgtgcctctgcccttctctctgtgtctctcatgaataaataaataaaatcttttaaaaaaataaacagtttaagtgatctgctaaaatattttgtttcatactgctattagtgttgaatacataaagtctggttttaaaaaaaataagtgaaatagggtgaaaggtgtggaattcaatttttattgtcccttttcttacctcctctctatcccatagagaaattataaaagaaaaaggaaacttattattcagaaacatgataaggagaataataaaaatgccaaaaagtcatggaaataattaaatgttctaatgtagtcacataggaaagaaaaaccagaggagatttttgtaaaacttaatgaaaaagaaagatattttataattaatcaaggctccagagaagtttttaatattaaaaaaagatgaaaaaagatacactcatggttattctgaatattttatttccttttaaggaattaagtaagttaaatttcctgtcttcttaattcccaaattgaaagtaatacagacccatctgtattttccatgtttctgtgtgtggtacaaccactttgaccatgtttcttcttttttcagtcttctgtttcttttttttatttttggatctttacttcattttctccatctgttctttctacatatataggtttcatatagcattgtttatatagaataaaattatctaatctcagatttgcataaaataaactaatacttttgagtgttaccattgtgcactttattgtttataaatcttacccttaaaaatgattagcagtttctgtgatcatggcacgtttatttggtctctttcctccctgctctttgttttttattgtgatatcttttgcctttggactcttttaagcactgtggtttgttctaacagttgggtcagtgctctccaacatttatcatattttcgtatgtgtatgaaaagataatattcatgtttcctatttattttgttaccaagggtgagatggaaaaggaaagaagcctcggttcttttcgctgccacagtaaagaattacaggacaacaaaacgaaagtaaaactgagcaaagtttcattaaagtgcacttcaagggaggagcgagccagtcaagagagacaagggtgggaaactctggggtgtcggcttatattaattttttcttttgggtgggtcctaggatcacagcgtttgagagacaggcctggatggtatcataattagccaacaaagcatgtccttttccaagaggtattcaaaatgtctgtgtgggataggaaatcggagcattatttccattctggagacaggcaaaagacaaggggctgctttcttgcacaaatgtaacaagcgctacaaacttaactcttctctgaaggaatactgtagtggcaaaatgactgctagaggggtgcagtggggtggtgcctgggtggtcctagattggggctctccctcaggatgtctaaggtccatcctgtctgtcatgcctagtttcctgcctaacaattttatatctcaatatttcatatttatcttgctttcttcactgtcaaatggtagatgtaatagtggtgggaaagctgctctttcacgaagaacttgaaaattttaattttcttatcatattaccaaacaagggattattatttttagcttcattaaacctaaagaagaagcctaaaactaagaatagcaatgttaggggtgcctgagtggctcagtgggttaagtgtccaactcttgacttcggctcagatcatgatctcaagatcatgagatcaagccccatgtggggctctgcactcagcagggagtctgctggagattctctccctctgcccctccgcctgctcccactcactctctaatccccaaattcctaaatacatacatacatacatacatacatacattctttaaaaaatagcaatgttaacaatggagtttttggagaaacaaaacagcaacaacacaacagaaacaaactatgcagaggcatttgtttttcttttctcctatttgagccacgtactgctacaagagctctggtttgctggtcagtgaggtcacatggcctttcaaatgaagtccatcctttctgtgtggcttttagctccccaagggtatagctccagcataggccatctctgttgttaacattagtataagcacatgctgtacatgagggcaattacaccacctcccactgaacacgtagtctctcattcctgcctgtaccattgtttttatgctgccttctttacctggagtaccccttcccatattttcagttaaaatcccagccatccttcaaggtcaactatgaaaagctgtttgtcgatgaattcatctcagatcctttctgcttgaaaaaatactttcctcatccctaactctattaaaattttattatttacttagggctctcaatcctttgtaactattttagacaatttcttttcttctccttatttaagttttaagaatgtaatgatggtaactttctttttaaaacatttcagcatcctaaacatccagaaaaacagaagagtacaaattgtgctttttaattgcaggtttgtaaatgtttattggtgcagttgggcttttctctttgataattttgaaaaggtttaccgcagtattcttgccaagaatacataacctgaatctaatcatgatgaaaatatcagacaaacccaaactaaaagatattccacaaaagaattttttgtactcttcaaaaatgctaaggtcaagaaactaaaagaaaagatgaattattattccagattaaaagaatctaaagttatatgaaagctatatacagtgaataatcttgaattggatgttggactaggaaatacagccatacaggaaattactgggataattgatgaaatatgattattgactgtattagttagtagtgttgtatgatgttcagttctcaagattttgaaattatattgtgattagataaaagaatatccttttttaaggaaatacatattgaagattttaggggtaatgtagcaggatgtcaactggcatatgtatacaacacatacatatatataaacacacgtatgtagatagacatatgcatctgtttacacagccatatgtatataggtatgcctatgtgtgtattgtgtgcatgataattggctaaatatcctgagttatctacttgcatatctgcagttctaaaaaatgaacaaaaggcaaagttaccacactcttgtgctaggtgctgtgtagctttgaaaaatgatgcttttcagggacatagttcagtgcatgcactgtgaagttagacctcatttccctgtgattggtatccacagagcctttaaatgtttatttcctatgtaataatacagcattctcctgccattcttactgtggggaaagggggaattatctggcatgaggcacgagaggtcaatctttttttctatgtaagattcagctctccagtcaagtaccatatcttatgtcttatactttgagtagttgtatattgtttccttcctagaaaaactgagtggttcagagacattagataatgattaggaatcatcaacttacaatgagtttctcctcaaaatttcctgtataagcagaatgtttaaaagagaaatttagtttttcactaaacaaattgtaaatacaaatgtctcacatggaaaccctgctacttgcctctagtcttgaataataggtattgcagagacctgacttaagtaataatacttttcacctgatatgactatgctgattagattatttagtacatataaagaactttaaagagtttctgctgaagaatcagtgcttaataaatataagctggttttattacttttctttctatacataaaatgaaaagaaggaaggagggagggaagtaagggtgggacggtgggcagaagtaaaaggaaaagagggagggagggaggaagagtggaggtaaaatgtaggaagaagaggtggaagagagagaagagagaggctgagagtgggtgagaaaaggaagatgagagagacaggcgtgctctaggactgtacataggggccaagatattaatggttgggcaagaagcagagatcagagtagtgaatggaaaacccaaggttgaaaagaatgtcaaggaaagaagtgggtcaggtaccaggtaaacactaacaaataaaggaggggttaaagttcagggaacagaaaacaagtggaaattgaggaaatagggtggaataggaagaggagcattgatttcagtcaccaaggctgataacaaagaatagctccatttatttcagcatttgcacctagtcccttgcaaggctgaggatgatctcaggtgaggccctgaagctttgggaccaggctcagaaaaaaacacctgcagggggctagcaaggagaagttctgagagaaatgacatttgtctttctggtaacttatcaattagataattgggtttgtttttctcactagctagatgtggtaaatatggagaaaagtgtatacatgttaaaagcaagcagaaattccattttcgagtggttctagttgttcattttcataattcgatgggtttggggtttctctaataagatgaataaaatagaaccactcctagtttattaaatgtttgtaattttcgaattttaaatatttttttaagagtaaagggaacttgggatgcctgggtggctgggtagattaagtgtctgactcttgatttcagctcacgtcatgatctcaggattgtaagatagagccctactttgggctccatgttggggatggatcatgcttgagattttctctctccctctctgtctgcccctcccctgctctccctctctaaaacaaacaaacaaacaaacaccacaattaaataaaagagtaaagggaactttactggttttatgaagtaatggaaatattcttgtgttgtttcatgaagtaaagagagccttcctacattgttccaactttatagaatagcaagaagaataaatataaatcattattcattttgtgagtctctatccatctcagactatgcaaggagtgagaagaggacttgccaaaatttcagatgcccggcaccaccctagtcctatttaattacaactctgggagtgagttgtgaaagcagagagcagaggtcttgagaataaacattaagtgagttccccatgtggctggtataccctgatgtttaagaactactaatctggaccaattttctcatgaatataatctctgttgatggataataaagcattgctcaaagaatagaaatggtggcattgcacattttaaatcagtatttctcctttgttgtttgaaaatttctaaattttcttttggaaaaggaactcatttatgtggattttgtaaatatgtatgcaattatagtttacctcattactttagaaagtaatgactaccttgaagcatgattcctgatcatttatactgatatgaaaaagttatttttgaaattctattcttgtattctgatgtgaaatatattattcatacagagcagagtatatgtgtataagcattttgtagagaaatttaaaaatcactcacctgcaggcttgccttcatcttaagatacagaatgttactaattcccttgaactctctgggaacttcttcccccatcatctttctctttcccattttctttgataaccaccattctccagtttctttatatatttttttgttgttgttatgtttatagttttactgcatgtgtatttatctataaaatgtattatctggattgcttgggttttttactttatattaatggaaacatattgcattaactcttttctgattcaatagtatgtttttaagacttgtccatgatgatgtgtatatagttaaattattttcattgcccataattggaatgcaccacaatttatctctcaactttattactcacataaattataactatttacagagcattgtggttttggttttctttaattgatgcaattacaatcctggtgcacaaatcttcgttttggagcatggtccaggaagagaattattgggtcacaggaattatgcatataatagttaatataatataaatagtcattccagcactgtagtaccagtttacctgatagaaatataagggtctccagtgcttcttattctttctaatacttagtaccttctgaatttagttttgcaatttagtggctgtgaaaggatattttattatagatataatttatactttcttgattactaatgggcattttcccatatttatatatttcatatgttcacaatctccaatttgttttaggaggtgctaccttgtgtcttttacccacctttttttgtctttgtaaaaatggtttataggatacttttaaaactatttgttacatattttacagataccttctcaaagtttaagtttggggcttcaatttttaacttactttttaacagttttgataaacatgctcttggggcacctaggtggctcagtggttcagtgtctgtctgtctttggcccaagtcataatcccggggtcctaggattgattcccatatcgggctccccgcagggagcctgcttctccttctgcctatgtctctacctttctctctgtgtctctcatgaataaataaataaaatcttaaaaaaaaaaaaaagaagaaaacatgctcttaatatagttaagtgtgtcaattttttaaagcttactatttattatatcctaagaaatctttgcttctcttaggtcataagtgtatcttctaaaaatttgaaacttttgactttctcattaaagtataatatccatctggaattaattctgtatgcagtatgaggttggcctccagattatattttccattgtgaattacctgttctttgaatgtttcaactagatacatttaagagttttggaaccatccagattttctatttgagtcatttttgttgttttatttttctaggaatttgctgatttcatttgttttcaaatatctctctataaagttgtttataacatttctttattttttatatctactcctcataaattgcgtgcttttctatttctaagatcatttatttgtgtctcctttttaaaatattaatagaggttctcattttttaaatgacatttgtcttttcaaaagacaacctttaaaaatttttttctgttgtatatttttctattttaattattttgtattatatttaatgtttgtttccttctgcttttttttgaattttttaaatgcttagtttatgggtatgtaacctttctattttctctttttacaataagcatgtaaggctctactttctctctaagcacttcttcatctatatccaacaggtttttacaactcttcaatgttattcagttccacctattttctgattctattttgattttgtctttagaagtataaataaacagaagtatgggctttaattgattttttgccattgatatctaaattttgttgtatgatcagagagtgcagtctttattagtcagatagtttgaaatttattgagttctatttatggcctagtattttgactgatttttaaaatattccacgtgtgcttgaaaagaacacttatctccatatttggtatgcactgttatttatggccattggatcaagcttgttatttatatgatatgtcttcttatactcttttattttgaactatctttatatctttgctctgtctttatagattaggtatatattttataaatggtacaaagctggatttttaaaattcaggctgagcaccttaatcctttaatgcataatttaatcaaatcctattgattatgatacctgatgtaaatggattcaattctatcatttattgtttttatttgttcactttttcatttttctcttttctttaatgtgtttttttttgccacctttttatcttgaggcccttcccattctagtggcatgctactttattctttagtgatatcctaaaaacttcaggatatatacttaaaatggtaactaaattattctcaataattgcctttaattattaactattatcttaaattgttatattgccttcatactgaaaagtaaaaaggccctttataatgctttaattctaattatctcttttccaattttacaggataattattcttagtttttctttccacattttttatatttatagaatcagtatttgtttaacttaaaccacatatttataattttctttatatggtgttctttcttgcatctcaaactacctttttgggatcatttttccttcatgaagtgtatctttaaaattttttctttcatgagctcttgaggtaataattaattacatctcaatttcaatgtgtgcaaaaatatcattatttgcatcattattgaagatagcctcactagatataggagttgttttctcccggttattgaaaatactattcttgtatctcacatccatgttgctgttaagttggttatctgacattgacaacacttgctctgtaggcaatctgtttttttcatgatcaactattttttttttaaagattttatttatttacttattcatgagagacacggagagaggcagagacacaggcagagaaagaagcaggctgcacgcagggagcctgacttggacttgatcccgggtctccaggatcacgccctgggttgaaggtggcgctaaaccgccgagccactggggctgccccaccatgatcaaactattaagatcctctcattgtatgtgggtttcttcagtttgtagtggtatttaatcatttagattttaattttttatttatcctgcatataattattgagctacttagatctgagattccatgtctttccataatttgaaaaatgttcagtcattccaactagtaaaatctttcctcttctccattctcattatttcctggaaccctgattaagcacatgtgcagccttctcattctcactatcttttaaccaccctttcacatctgccttccctttgtcctcttgcttcattttacttaatttcttcagatttatcttttagataacaaatttctcaaccatttctcatctgcctgtgaaaatttcattgagtttctaaattcagttattactagccattaacattttatctctaagagttccttttggatattttatccaaagcctagcccatcctaataatctttgatttctccacagcctcatttttctttttttatttctttcattatattaacactttttatattcttttcctggaaatagtaatatgtggattgctggcctggttttctaagattttctgttatttaagtaatttttttcgttgtaattgatgtaacatggaactttatctatggaaaccctttgaggtctgattttaatgtattttgctaaagaaaagatttcggactgtttctgccaggaactcaaatttccacctgggaccactttaaactaaatttttggcttggggtttttttggcagttttttcagtagaatctatccaggtcctaaacccatgtgaatgcttgtagtctgggaaatcttaagaagcaatctttctgcattttaattccaccttgagccaaggctgagaaagaacatttttttttttttgcgtgcaggttttagtgccccaatttataacaagatttctggtctgatctccattttgctcatgcctggacttttctctttcaatgggtatggagctccttaaaacctagcaccgctagggaccagcagaaaaaaaactcagagcaaaaattggttccaatgtatatccttaacaccaggaatttgagcctatttattgtttttgtctctgagaaattttctacttcagcttcagttagttattcgtgaatgaatgttgttattaataatattactgttcttaatattattatttgggggtatttcaggtttaatatatagggctgggtacttctgaatctctagtctgttatatttctgtaaacagattactagaaatacttattgtaaattgtaaaaaagaatcattatcagcgacaaatataataaggactgtgaacaatgagaaatttcagctaattaaggatgcatcttgacttaatggcctccaggtgcctcggaaccaaatagctaaaactgccttttctctgttttttctctagatgtttgattttcattgtcacaatccacccacaaaataaaaatccataagcctgagtagattgttagtgcaaaatttgtagacattccaatttgggaggaaaaaaatgttttttaaataaaaattcttttaaaaatttccagtgaaataagaagatatatttaaacacatataaaaaaaaacatatatgaggaaaggatttcttagaaacagtgaaatttaatcaatatttcaaaataagccaactacggttctttaaccaaaaagaaaaaagataccattataaataatgactgtgttctacttttcccatcatctgtcagtcataaaagataaaaataggcccatgttattgtaggaaagatgtgatataattaatattcatgttttgttggcattcatgatactgcattaggtagttgtgttcagagtgatatcaaagagaatactagtttgttaatagtgtacctaatagtgaagaaaacttactgatttgtaataacagcaaattagaaataaatgtggtatggcatatcaattaaatggaaacatttgtattgtgggtttttgtttcttgttttgtttttgtttttgtttttgtttttttggtttttttgcacatttctaccctggggaaaccttttgggagagcttattttatgagaaaccgtggtgttctatagtggaaaggatatccagtaatcatgcattttctgtgtgctggcctttggtcacacatgtttatagaaatgtggcatagactctacttttggctttgctgaatcttcttctttctagtacattctcctccaacttattgagttatgagggagttggggcaggagaggctgtatgctgaaatgaaagtatagctaaaaaagaatgttgtagataagttcatgaactcttaaatggtaactaaagagtagtaacagtgaagttcattactttagtacactacaactttctttaaatctctagtcatgggacgcctgggtggttcagcggtaagcgtctgctttcagctcaggacgtgatcccgcagccccgggacggagtcccacatccggctccttgcacagggcctgcttctccctctgcctctgtctctgcctctctctatgcatctctcatgaataaataaataaaatcttttaaatcaatcaatcaatcaatgaatctctagtcaattgctgattggaaattgtaaaccttatatagggtgactgtttttcagccatgtaaatgccccattcagatcatttccggcagttccactcttgcataattgtttactggattagaaagaaaacctttgaataattgttgtctagtcacctatttatagcagtatctgttactcggaaatctaaagagataaaaagtgactctaggaaggctcaattacttgtttttatttatgtaaactcttttctttgggagtatgtgacaacctggacaaatattctgatgctaagacaaatcggtttcactgagaaaactgtatgtggctttaattgacagtaattcatgtgtaggtgttcttgtatagagagactgcagagctagtttgatctatgcaatttgaagggctttctcatgcttctggggagtttgaattttaacttcaaagcactggtttcaaagctgtctaaagtgtggataagatacaactggacaaggattttaggagaatttctctgactgataatgactgtaatgactggacaaggaatatactgcaatgatacagaagagaagcagtgagagtctgaatttaaacagggggatggattctagagaaaaaattggagatgtttcaaggatcaaataaacaagttataatggggcacctgggtggctcgtggttgagcatctgccttcggctcaggtcatgatcccggggtcctgggatcgagatctgcatcaggctcccagcagggagcctccttcgccctctgcctgtttctctgtctctatctctgtgtctcttaggaatgaatgaatgaatgaatgaatgaataaataaataaataaataaataaaatcttaaaaaaaaagcaaaaaacaagctgtaaggacagatttgatgtatagatgttgagcaactgctggaagttaagaatctctcagatttctgtccttggcccttttgttctgactagctctccctaaatgtggttaaaacattttgtcgaatctgaagactttcatatttataatcttaggccagtgattcttaaactttttgatctcaaaatgacttattattcttaaatttttttgagaaccccaaagagctttttgtctgttaaatctatcaatttgtactatgtcagaaattaaaatgtggaatttaaaaaattaattcattttaaaatagtaagtacattacaggtgaacataaataacatatttctatgtaaaagggctatttttcaaaagcaaaaatcaattggtgagaaaagtggcattggtttacatttctgtgaaactctttaacatctggtttaatacgagaattagattttcatactggctttggcagcatgttgtatgtaaataaaatcttgcctcaccctgatacatagttagtaaagagagtaatattcttctgtaataccacactcaaacttgatgagtggtagtttcttaagtcttacttgcaatgggaatataaaaatcctatcagtgaactttttgtactttgttaaattacagtcctttggcctatcttgcacttcagatgaccctttacacacatgatttgtaccatcatacattgcacatgtggatattggtttaatgagatgccaatattttaaggattgataaatttcactataaagcatcctcaagtcaggtttattaatttactgttgatttgtaaccagaaaagtatcaagtcctgtggttgtcaggctcatggtatgcatatattttccaaatttctctttttgcttaaaatacagattgatctcattgccagcaaatattgtcaattttccttgaaatgacaagtttactttatcactttcaagaagatttctgctggtacttaaatttgaatagccatagtttgcctattacttgttctttctagtacaaatgtttcgtgttgtcaaggattaggtaagagggagcccaatgacttttttagatatcaactcaaagaattgtgcaggtgctttttctgaaataatgcttgcacttttttatatagcagaaatgcaatatgcattttgctacataattaaaaagatgtgtactggacttttccttcttgctaaataaagtaattagaattcaatactgataaacaaaatgaacaacatatatgaaataattgtttgcagaaattggacatcgggcagttcagggatattatacagagacagaggaagcagtaaagggagtcctgtagttgtcctggtttactaccaggagagagattttaggccacaacacaggaacctgccacaggtgcagtcaatggcatcccagagttcagagatggatgtgggagcacaatgaggctgtggcatctcaagttagcaggcagagttccagagcagtgaaagctgtgcagagagggagctgtggagaggtgcagaggattccccttgaatcctcacctgaatactcactgtgacatgcctgccaggaaacgcctcaagacctatagaaaaaccatcctgaggaaatagaggaacatttcaggagatcactcagagctggaaatagttttgttgccaccagcaagagtggaaaaacctcctaattcaagaaacattgggtagggtatttagaaaggtattgcctgggtagtggaggcaaaattagccacagattaaaggctcttctggccatctctcacaaaattggaaagcaagctttaaaaatatcaaagtgttttcaagttacataactgcatcccagagcaaagcttgggaatatctgtaggaatatagcacccaagaaaataaagttcatgatgtccacaagcatgcaaagaagcaggacaactttctacataatcagtagaaaaattaatctaaaagggtttatatccagaatatatatagagaaccctgaaaacacaatacaaaaagaacaaacatctaatgaaaaacaactgggcaaaagatactttgaaaaaaaaaaaagatatccagattgcgagaaagcaatgaaaagatgcttcacattattagtggagcacaaactacaaaactgttttcccaagtatttgtatcattttacattctcaccaactttgttaagagttccagctgctcaacagcctgtcaacacttggtcagtctttctaatttcagttattttcatggttgtagagtggtatcttactgggttttccttcttatacaaaatcaacatgtatttcaaccacgagtgcacggtggttaataccataatttggcaccactgcctttatcatcattgtttaggtgacagttgttctacccctcattgcttttgcaccatcataggtgaatgatatcttagtattatcaccaaaattgttttgacctcagagactccttgaatcactgttctaatatgcactttttcccctagatttcatgttgtttgtgtttttattttattttatttcttctgctgtccctccttaaaaaactgccctattcatatgttccatattttaaaggcatcacttctatccccattttctgagtgctatcacagagccctctctcccacattcaagtaatttctttgccttagaaatatctctgcttttccattttccatcctcattgacctgtatcaccatctttctttttacagtctgctgctggtggcttttctcctctctttctgaaacacacacaccaacagaataaagccaaagtgccttggagcagcaggcacttataattaaatgttttgaaattaatagcatgatttaaaacacatacattgattgttcactaatttagacaataattaatcaattctaaccgtaaatgtgtaatgataacatagtatatttaggacaaaggctggtagaactgtttttgtacttccagaatgtattaaggtttgagaaattattagctgccaacttttagattcaagggttgagtatctgctttttttcctttccctaagaaactaatagttcattataacttcagaattccaattaaaatgatggattgtttgaaaagtaaattactcaaaaagtctgaattataaatgtgagttcaggtatgtgatgctctacacagggttgtgttttgtaccagtaaacttctctgtcttagatgatttaatgaagtagctcaaattatttctgaggctgtggagactaagagttcaaaatgtttttctcactttatgatacattttcaacttctctttaaactttatctttttaattttaatcagtttatattttgaattgtttttattttattttgtatcttaatgtcagtcaatatatatgttgaagaatgaaattcaaacaccaaaatcgttagcagaagcttattagtcctagacaccagagtgtaattacactctatttattacaaataaataaaagacatttggtcagaatattttcagttgtgttggcaatggcattttaaaagatttattttttaataacgttaataaaataagtgagagagagagcatgaaagcacaagtggaacggacagaagaagagggagggagaatctcaagcagactccatgcttagtggggagcccaacttggggcttcatctcatgactctgagatcaggacctgagccaaaaccaagtgtcagatgctcacctgtctgcaccacccagggtcaccaacaatggcactttaaacactatgcttttttatcacattcatattgtaaatgtattgcctgtcttcaagatttctagacctcccttttttgaatcaaaactaaggtgtactgtttcatatcaccctgaataattattccattgactacatactacttagttcatgtcattcctcgctactccaaaaatcctattctcttcatttttgccgaaagctggtcttttccatgtttcatttacacatggaaatgctcatctgctgcattaacagcaccaatctgaggctttctgaacattaagtccagtgttaaaggaaatatactatgtataaaacagaaaaacaaaagatggccatttcttattgcagcaaatatatatctagatttgaagtgtatttcaagaaagtcaatatcttgaagcctacagaattataatctgaagtcaataaaatttccgtctcaagtgaagattgagccattggagggaaattttacacatgaactgtgttttaattaaacatgccttattctagctggtatttagccatttgtgagtcttctaattattttcctaggcagacactttaagttttatttttttaacgtcagtaacattttcctatccacacttaggtttatgctggaagatcaagcacagaatcatatcagattaagtaacagcttgaaacaaaatgatgtatttagatcactttcccatatttttatatctcacataggaaataaagtgagaggtccttatattttaacttaatttttcactgggaaacacaatatgcagttttaaattatattgagaaataaaaaaagatttaaaaagtcagacattttatgatatcaccaaatgggaattcacttaacactcatatattgaggatgtatattttttacattaagtttgcatgagagatttcaaattacttttactagtatcttaatactcctatatattgggagagttaaaaaatttttaaatcaccacttacagttgcataatagcagagttggaattacaggtcttcctaggaactatatccattaactttttatggttgtactctttactaggcaattaggattttcttttgaattgtaatgggaatgtttgggcagattgaaatcaacagaagtccttctcttaagtttattctgtatctacatttttttttttactgttaatagtatcataatgctataaagtgtatttatagggacataagtaatgctcatcagtctctttaatagcttcccacatgaagcttaacttttattcaacatttccttttattcaagcacctcaacaagaattcaatttgactaacattatagtaattgagaatgtatttttcagccctgtttgaatgccgcccaatcccccaacaaatgttctatcatcccttttctatagtctggagcttcttaagtaaaggattttctcaatcattgctccattatacacaatgactagagccaaacacaataattttgttattcaactggccaagttctttataactcatttaaattctttaaaaagacgagaaggtaaggttcatgtctaatctgtaaatgtttctttgtctttcaaagctgtttaggaggtaggaaaaaggagctatgcagttggagtttttttcctactttccttcttaggaaggatattatataatttcacatataattttatggcttttttatctcaacaaatgtattaccttgactttgaattatgggtaagaattttctttttttctttttatttttttatgggtaagaattttaaagtaaaaaaaatatttaaaggttttattctttctaattataaaaatgatatgtgcttatgtaaaaatctgactagtacagaaaatataaaagaagcacacacatgaagaaatgtcactcagactgacaatacctggaggcaatcactgttaacattttgacatttttcatttcaaagataaattcttatatgtagaatataagatacttaaagttgagagaatatttgccaaatgtgtacatacaatcattttaacactaaatcataatttatgttcaaagaaaaggcaacacatggtaagcatggaactatactgctgaaagaatgtgggaataaaatttttttttaaagttttaaaacatctgcaatgataggccagaatgtcacattttgcctttggcttgggaatgatatattaagaactgttcaatgagaaaatgaaaatctccccaaaatcaatgttgtactttaatgaaagattggaacaaatagttcaaaataataaaacaaaatattaatatatcgaagcatagatttaaatggtgatgacaattacacagaattgtccaaaatattgatgtagggagaagcaaaagtaagtgtttcagggatgagctacaaacaaataaactattatgaacaacggaagaagttatacaattataatgaagatttttctaatacttcatatgaggaattattttttaaaaaccctttttataaataaatcacagagttaaaagttagagttttcacacttaaaactcatctaaaatatcttctccaacattgtttttgacaaataatgtcattagatcccaagacattgcattatttgtgcaggtctcatgcttgtaagagtggggaggaatctgggaaaatccaagagagtcagaatgttggtgcctcccttctgaagtctcattctcaaatggtgacttgcatgagaaagcacattacttctctttcattctttgttcatgattgatccacttattccatacattttcaacaaaaatattttctaatgttcacttatgaatttctgtaagtcatgcactattattataatttaatgtaaatatgtatttaatttttacaagatagatgaggtctagatacttgtaccattctgatttttaagcaaaaatacaaacataggagggtaagcgactagaaaatgacagaactggaatggaccagatacttccagtgcccacatttcaaataaatacaccagtaaacaaatttggggttgactttgaaaaaatagcttttagggtactttgggaataccagaccatttttaacgatttggatgtacacatactcagctctaattagagtgtttttaaaattgtcagagactaggatgaaacctgaggtaacaataactagaatgagctttgcagaggtcagggctgtcacaagcagtgaatcttcttccctgtcatctatattttcaattgtggggagatgttaaggtttcataaggcacctctacattctaaacttagtaagagcattaggatttgacatattttgtgtctcaagccctgcatcttgattttcatttataaacttgaaatatgttttatttgaatacaatattatgcagttcttccattccagttatctttctcatgctggatgagctttagaacattttcctgtctgaaacagtataacactatttttaaaaaatataacttttaaaataaataggtttccgaaattttccatttgaaacatttcctattttatccaaaatgtattcttctaattttagatatcagatttcaaaattgcaaacaaaaaattttacccacttttcctgatctaccatcttcagagtatgtgtatctccttcattgatctcattttattatagtaagatttatttttatgtttccttatcactattatttccctattttgtactttttatgataaatattctatcatattaactgactcaataaaatacaatggtacccatagtgccttcttcatcagaatattaatcataccaaattgcattatgcacttagacactttctttgtatcagtcttttgatcaccaggatgtattttcacattgttaattgaaacattagaatcaagtatttattgctttcccctttattgttatatttatatattaagaatacatttttcccataattctgaaggcatctggatattaggaatacttattccataacctagaaaattcatattttctgtctaatatgcttctttacttactatgttagtatgaaacctgtattatttatttttcttctttactcaattttgaatgattttcttttaaaatatatgtatttactataaagatccttcgtttgcaataatggttttagccaattgacatttatggagcttttaccatgtgtctgatctttgatagttacctgatattgaggtagaaacaagattacaagttgaaactatcaagagctcactgtgtaatgaaagagataggagaataaataatatataagtgatatgatctatgacactttctaatgtagtacatcctaagagctactattaccaggaagttgggaaaggtcttctactgggagtaaacattgatgggtgtgttgaaaactaattaggaggaaaaacaggactaggtacaaaaggcaaaaagaaaagagcctatgtgaaagaatgaaagattatggttatatttggggactggaaaggagattagaatggctgcaaggggagtaaagaattgtcacagaaaaagcaaacgaagtgggtcattagggctccccttagctcttaacagaatcaagttggctgatgatatcagcttactagtactgggaggggccagatgtatgtggatcagcaaagggtgtttccctttagtggaaagtatagctctgtggccaagagagcagtgtttggaaatcaaagagttttgctccaactctgtgatctatgacaatttatttacccttttaaaaacccagttcttcttcagtaaaatcagaaaacaatagtatgcatttcttaggataattgtaaagatccaaggaaattatgtatataaatctctacccagggtatttcagacttaatccaaaatcaatgcatgttggttcttacttttacccaaacccaaaaagacttcctttagggtcttatttgtcaacaaagtgatccttccctcaaaaaaactttttaggtgtaactgtaaaatttctttctcattttaacatatctcacagaataaatgaggaaaaaagcaaaacctatttctgccccaaaggtgggtagattcaatttctatactttttcttcaagtgtaagaagagtcatgcttatatataatatttctgggaaagcggaagtttcactcagaaagctgcttttacatcaaagcaggcaatttcaaagtcattttgtctagtcacatgaaaagatgaaactaaaaactataacacatcctaaaattatttacatatagcaaagaaattttttatttgatttggaagtgaaaataatgtcctcaggtccagagtctctgctactttatggaaatagtcttaaatacaccagattaaaatgtcacttattatttaaaatgtgttaaatgctggtaaacatttttctaaaacatagcaaagaaagtttgtcatcaaaattcaagttatactcttagataattgtaaagaatttgaagatacttgaaagaaaaaaatcagaaatgtatccttctccttcagatccaatttaggctgagaagaatcatcattacctactgataatgattccacatgtttggcattataaaaagagtcattttatctggcagcaagagtcaatccaacaacacgtaaactgctcctgggaattctttgtaaaataaaacattcttgtgagataattcagtgaacaataagactggtaaacatacactctcacacataaactgccatttctatgtaaattagctatcctaactggaatatcaatttaatgtcaaatggaatacagtcttatgctatcataagaattaatgccttagacattttcattggagttttgcaccatatgttgtttttaacagctctgaaaattttgtgtctttgattctatcagattgtccctgtgataaattgccatatgcatgatttcactctgcggggagagacagtatagtgtagtgatgaggagcctctgaaatctgactagctagctttctctttgtcttcagcaataactggcagtgtggccttggaagacttaactcactgcctgggctttcacagcatctaccactgtccgccttttactcaggtattgtttgcctcccccaaacccattagattgcagtctgtctgggaagtaaggctcacctcagcatcctgcgtaacacttagcacatagttagtgcttcataaatacttgttcaaagaacaaatgaattaatatgtaaaatgaggatgatatcacctacttcatggagttagtattaagtgtgttaatatatgcaaagcactaaaaacactacttagcacatattaaatgttcagtagttgttaggtgttgttaataatcctaaggaagtttgtttataaaaatcatgggtattcattttagataaaatctctttttaaaactctcttcctttgaggcaattaattgaattgattatagcttttcacttactaaacaccatgactaaaattccaggtctttgacagtaatttatgactgatcttgctacaatgggagaggaaaagtcaggagttacatcaacttgttaagacagtattttcactcactgatgatgtaagagaaagtcaagtccagtgtcaagggcatggaatttgaagtgaaggtgatattctgatattttttttcatgcttttattatttaatttaatctaattcatggaagttaaaaaaaaaacaatactattgacttaaatttagataattctaatgtataatctcttactctctcagggaaacttcttgggcattatagaattatgtacctttctctttctagtctgtctttctgaatcttgttcctgttcttctaatagcttccctccttctttttcaactgttgagtcattcttcaagtctagctactccacatgctcatgcttatgtctgctgagccactgatgaagtcagattagacatagtttccttgctcttgcttccctgtacttaggcttctgtatctcctgaaacctagcacactggatcctgtagctatgtgtaaatacctgtaccaaaggcagcagcaaaaccaaaagcattataattgccattaaaatggatggttcccttagtttcaaaggtaagacaattctatgtagacccatcacaaagagaggctggcgcttctctgctcttaatcaactccctctttttttttttttttaaatttttttttcaactccctctagcctcatcttcttcaagcatttccaattgaaatatattaattcatctattgcacaaattattattgatcatttgttatatattcaacaatgaataagatagtcagttcctcaatctcatgtagattaaagaaaagtgtgataggagcatggttccagtgactacaatgttctcctctctccttttcattgttttatatttaaggcccattctgagaggtcagtctgaatgcttgattttcacagtggtctttatcttaatggccaacatgtggaacattagtattcttagttaggatacacatatgcacagagtaatatctctctagttaaactacttaaagtttaaggaggaataaaccaaacagttttataaacaatactgaaattttattcctacacaggacaagaggatcttcatcatttaatttgcaaaatttaagttttgaatttactatttatggcttctatagaaagtaatactttcttcctgggttaattgtatctatttttttgcttttttaactctgatacaaaatactgcaaaatatcatctgcacaatttttaaatgtaatataagaggagtaagagagcctgtctagctgaaaaatttatgggggaggaaccatttctgatacagaattttaaaatctctgcaaatagttttaaatggttgacataaattcagtaattcagcattgaacagggattatagttttatttacatatttaaaaaaatacaatgtatgtgaaaatcttgattgcaaagagccattgggatacagattgcaaagagccattggaatacagccacgccttgttttactgtgctttgcttgtgttttttacaaatcgaagatttgtggcaaccctgcatcaagcaagcttatcagcaccattttcccaacatttgctgactttgtgtctcaacatcatattttggtaattctctcaatatttcaaactttttcactatttttatgtttattatgatgattggtgattggtgatctttgaagttactgtcgtaatttttctgtggtgccatggaccacacccatataagatggtgaacttaaatggatgaatgttatatgtgttatctctgatccacccaccagcctccccatctctctccctccctttgagccttcctattccttgagacaaaaccatcttgaaattaggccagttagtaaccctacagtggcctctaagtgttcaagtaaaatgatcattcatgaagttggttacactaaacaacagatttgtaatgtagatgaaattaaccttttattaaaagaaaatgccatttaggactttcatagttaaagaggagatatcaatgcatggctttaaagcgtcacaggacaggctgactcttgtgttaggggttaatgcagtgactctaagttgaagccaatgctcattttaccattctgaaaagtctatggccctgaagaattatgtgaaatatactctttctgtgctctataaatggagcaacaaagcctggatgtcagtacatccgtctacaacatagttactgaatattttaagcccaccgttgaggcctagttctcagaataaaagatttttttcaaaatatgactgctcattgacaatgcatttagtcattcaggagcccagtggagtaatacaatgagattaatgttttattgtctttgaacaaaacatccattctacagcccataggtcaagtagaaattttgattttgaagtactgtgatttaagaaatacatttcttaaggctacagatggatctgagcaaagttaattggaaattttctagaagggtccatcattctagatgccattaagaacattcgtgattcatgcaaatatcatatcaccagagtttggaatttgattccaccatcatggatgactttgagggcttcaggacttcagtggaggcagtaactgaagatgtgatggaaataacaagaaactagaattagaagaggagcctgaaggtgtgcctgaattgctgcaatctcatgataaaatttcaatggatgaggagttacttgttatggatgagcaaagaaagtagtttcttgagatgaaatctacttctggtgaagattgttgcaagtgacaacaaagaatttggagtattacctaaacttaggtgataaagcagcagcagggtttgcaaaagagttccaaatccaatttggaaaaagttctactatggataaaatgctatcaagtacaatcacatgctagaaggaaaacatttgtgaacagagaaaccaatcgaggtgttaaacttcattgttgtgttattttaagaaattaccacagtcacacaagccttcagcaaccatcaccctggtcagtcagcagccatcaatgtcaagacaagacgctccaccagcaaaaaattatgacctgctgaagcacagataatggttagtaatttgctaaaaatttaacaaaaatttttaaattagggtatatactttttttagacataatgttatcacatactttatagactacagtatagtataaatgtaacttttatatgcactaggaaaccaaaaaaattatttgacttgtctttttgcaatatttgctttattctggtggtctggactaaacccacagtatctccaaggtatgcctgtataaagtattattattatacctggtaatataaacacatataggattgtcccattttacttttcaaaaatatgcatatatattcttcaaacaaaggctgaaaggatatatcaaaaagttagctgtagttatctctagaaattgaaattatatgtgattttattttcttctttttatttgtgtgttctaaattacatataatatttattacttttgtgaaaatacataaatttgattatgaaaaatggaattgctgctatcctcttatccaataaaaatgatgaacaatttttgatgttcatttttatctagtgactagaaaaataatatttatgggttgaaagtctagtgtatattataaagaatgaaaaagaaaataacttttattcccacctctcagaaataaacagttaagataatggggatttttcttgtcctattatatattatgtattatgtatatgtatatgtagaaattatgtattatgtagaaataatatatacatatacacttacatatatttgtggatatacatatgtatgtgcatatactgtacacatatacatatctaggctttttaattcagataacattataacaaaaattacctcatgctattgctcagactatatattttatgaatctctattgaaattaatctgttctttttcttaattctaagaaaaaataacaaaataaactggaaaattgctgaatatgtcaagattgagtgtgcattatcataccctaggtatttcaatggaatattgtttaaaacaacaaaaaaatctttcctctattcatgaattatatgtcccgtgtataatttaatactaagggttggtatgatcaattcatacaaacatttttcttgtagcttgattttgaaaatatagcaaccaaatagattgaaaaccttcgatgttattcattaaaggtaataattgaaagtctgatggtcagttttgtttgcaatcttattcaacagatcttgactttatttcattctggacaatctgcttaaagtagttatcttgatacatgttgaacttcttcagagaagatatagggtttgtttcattatctggccttttaattcctttagctcagcgcgtcatatacccagatataggagataatgttgatagaatgatgatatttaaactgccttagcaactctcagaaaacataacaatgaaaatgtagtggctcagcggtttagcgcctgcctttggcccagggcgtgatcctggagacccaggatcgagtcccacgtcgggctccctgcacggagtctgcttctccctctgctgtgtctctgcctctctctctctctgtctctcatgaataaataaataaaatcttaaaaaaaaaaaaaaggaaaagaggcaaatagtgctttctaagctatggtgattttctctagctttgttcagttcctatttttagagttatacctactgaatcacagatgtacctttttcaaacagtcaggcatagaatgtaatagataaattaactggccttgagattcaaaccactactacccccccaacccccaacacacacacacacacacacacacacacacacacagctggtaataaaaggtaggcatggatttattcaatcattaaatcaggaaacaattacttatagcacctactctgtgaaaggcccttttgtatgtactggagacattgaggtgaaggaaaagagctaattgccttatctcaaaagcttgacttctagatggatggacaagagacaaggaaacatgaatacatgatatcatttcgattaatggtttatgatatgataatgtaatgaaaaagtctcatactcaggtattaattattactgcacattgcttctcaaacttcagtgtgcattcagatcaatggggatcctcttaggaggcatattctgatttagcaggtccagggcagcaggctaggagtttgcatttcttaacaggccactggtctgaagaccacactctgagttgcaaggtatagtatcatttctgcgcaccaagtttgactaagaggtcagtaggaaatggccccttaaaggaagagtgggtagtggctggaggattaggaaaagggaagggaaaggataggaaaaaggagaaaatataataaagaaaatgggatggaaatgcaggtaaaaatggaacatataggccaccaaagaaaccatctccttacatatgctttgcctctgcactatccttgcatgttgtgaactaaaattaaaaagctaaagtgtctagagtcaaagactcaagtataaatgtacattcatgctaacgtttgagagaatttgagctagtagtttgtgaaattagagtgccaagaaagaaatggacatttaaaataaagtagatttatcttcttattggaaaccaacatttgcttggctatattaagtatatttattttttatgtatttggaagtaaggtgttttattgaaggtagcattgaacaaaatatgtcttatgtccattataatgggcaacatacaacaaagattgtttaagaatgaggggtgctggggatccctgggtggctcagtagtttggcgcctgcctttggcccagggtgtggtcctggaggcccgggattgagtcccacatcgggctccctgcatggagcctgcttctccctctgcctgtatctctgtttctgtctctctctctctctctctgcatctctcatgaataaataaataaaatcttaaaaaaaaaaaagaatgaggggtgcctgggtggccccattggttgagcctctgccttcagcgcaggtcatggtccttggatcctgggatctagctcagcgtgaagtctacttctctctctccttccgtccctcctgctcagtttctctctctcactcactctctctcaattaaaaatacatatttttttttaaaaaaaaggttaagaatgctagtgactagaccaagatgttggcaaaaattacatctctttttacagtacatttatttttttaatctcttgggaaaattatgtgtttacttacctgaatagtggacagtattgtattcttggttatgaacattaaaactccagagaggcatgctaggttttgtacttctttaggcacacttaataaaatcagcagtattgtgattatagaatcatttaccaagattataaatttgacaaaaacaatgatgttcagttattgaaattacaaggaaatattatagttgtttctaaataatagttggattataaattgagaaagattgatataaaatattttgaaaatataaaatgtttgtatttattttgctacaagaaatcagaaaaaagagaaggaaactaaaattgaatgtctgtttaaggcctaacacatcatacatattattaaatgtagtgaaaacagtccaacaagaaaggtactatttatcctgttaagtaattaaggacactgaagagtacatatagtacctttcccaaggttacatgagaggacagaggcttttattagcagtaggtatatttctcctacattaatttttgttattgtcgttgtttccaaatcatttgtgatgaaaaaatgaaactttgattttgtcagttaactattttcaggtttgaagagtctttgggcattgaggaatagccattttttttcaaatcaacataacaaaagcaattcaaatatgatagaaataaaagattccactttttttagtcttcacctagttttattgattatctgaaaacctgtcctcttgccaaatgaggatttaatcaatagacaaataaatataaacaaaatttttaaaatttaagctaaaaaatatctgttttgaaaaaggattattaaagggtgtctgttttgaaaagcatatggcaagctttaaaaattacatataggggcactgagtggctcagtcagctaagtatccaactcttggttttggctcaggtcatgatcgcaggattctgggatggagccccgtggttggactctgtactcagcagggaatctacttgatattctctctctcactccctatgcccctccactcctcttctctcctctttgtgaattaataaataaataaaccttttttaagaaaattacatatatatacctatttttgtgacttaattcataaaggaaaatttattttctagagctttactatttaacaaagttttactaagtttacagtgcattttttatggttattaagaaaaataaaattatcttgcttaaggtatctgtgattatttttgcctaatattaaatttgctttattttattttaaaagatattatttatttatttatgagagacccagagagagagagaggggcagagacataggcagagagagaaggaggctccatgcagggagcccaatgtgggactcaatcctgggactccaggttcatgccctggggagagagttctgagccactcagaagtctcattaaattcattttatttatttatttatttatttatttattattattattattattaaagattttatttatttattcatgagagacacagagggagagaggcagagacacaacacaggcaaaggaagaagcaggctccacgcagggatcccaatgtgggactctatcccaggtctccaggatcaggccctgtgctgaaggcggccctaaacttctgagccacccaggctgccctaaattcattttaaaagaggagattcatctttattctagatatatcttttagaattgcttagatcattttaatatagagatgattagtgaaaagctgattcatttcacatactaagtacacagcagtgcttcctacccttttttgtcatgacctataaagtaaataagcttttctgttgtttcatttacatgtctagaggaggggtaaagaaacatgctccgtctaaaatcttgatcagtttattttattcatcaattattcttgatcattcaaatcattcaattacttgaggaatgtatattgttcaatctgtaaaacaaatacattttcattatagaaaaccaaactatatattatgtatattagatattattaatattattagtaatacatataatgaaattacccataaccctaccagtcagaaataaccgctcttgatattaatggaatatatatatatacatacacacacacacacacacatatatatatatatatctccaatgtattcttatgcattcaggaataagtcttgaacccctaccacatagttctgtatatgtatactttctttaaaagactttattattttttagagcatttctatgtttacatcaaaactgagagggagatagagatttcccgtatactccctgcaccccacccctacaagatgcataggtaccactactatcaacatcactcaccagaatgatacggtttttaccatttaaggatggatctacattgacacatagttacctaaagttcgtagtttactttaggttatagtcttggcattgtatcatctaggtgtttggacaaatatataatgacacctacctttcactatgatggcatacagagtattttcactgtccaaaaaatcttatgtgtttcgcctgttcatgaccccgccttaccccaggccctggcgactcctgatctttttactatctgtatagttttactttttccagaatgtcatgcagttggtataattccagaatgtataatttggtataattataccatgcagttggtataatttccagaatgtcatgcagttggtataataatatgtagtcttttcaaactggcttcattcactcagtaatatgcattaatgttcttccatgtcttttcattgcttgatcatttaatttcagctctaaataatatttctttgtctggatgtaccacagtttatctgttcatctactgaaggacatctcgatcgctcccaagttttggtgattatgaataaagctgtgctataaacatccagtgcaggtttttatgttcacataaggtttcaactgatttgggtaaatgccagggaccatgatggctgaattacatggtaagactatgtttaatgttgtcagaaaccaccaaattattttcaaaaatggcttaccgtcaatgtgtgagagtttatgtttctccaaagtctcactaacatttgacattgtcagtgttcagattttggctgttctaatgaatagatgtgtactgttaattgtgtgttttttaaattttgcatttacctgatgacatgtgatgtgaagcatcttttcatatgcctggttgccacctgtacatcttctttggtgaggttaaagctgttaaagtttttggctgatcttttaatgtatttggtaattttcttatatgagtttaagagtttttttgtatattttgcagaacagtcctttattagatatgtcttttgcaaatattttctcccagtgcatcccttatcttctaattctctatatactatcttttgcagagtttttttttttcctttttaatgaagtccagcttatggataatttatttaatggatcatatcttttgtgttgtatctgaaaaggcatcaccatacttaaagtcacctaagtttcctcctatgttatctaggagttttatagttttgcattatacatttaggtctgtgatccattctgagttagtttttgtgaaggttgtaatgtctgtacgtagatgtacttttttgcctgtagatttccaattattccagcaccacttttttttttccagcaccactttttaaagagactgctattgctctattgtattgccttttctcattagtcaaagattcattgattgtttttatgtgactctgtttctcagctgagctctctattttgttttaatcatctatttgtctgctcttttgccaagaccacactatcttcattactgtagctttatagtaagtgttgatgttggatagtgtcagtcttccaactttgttctttgatattgagttggctatcatgggtcctttgctctccatgtaaactttagtatcagtttgctcatatctgattctgaatgggatggtattgaatctatagactaaattggaaaaaaaaaattgacatcttgacaatattgggtctttctatccataaacatagtgtatctctccatttttttagttcttatttgattttgttcatcagagttttgtagttttcatcatttagctagctcttgcacatattttgttaaatttatacccaggtatttcattttgagggggctaatgtaaatgacattgaattttttttaatttcatttgttagtatatagaaaaacattacctttttttttgcatatgaaccatatatcttacaacattgtaaaattgcttattagttccaggaatttgtcagtgcttttggatttacagcatagacaatcaaatttctcccaataaagacaattttatgtcttccttcctaatctgtgttttttttttttttcaatttcattgtttctactctaattctcctctaatttggtcttttcttctgctttccttgtatttaatttgattttcattttttagtttcaaaagacagaaacttgaattttattttagatcttctcttctaatatatggactcaatattgtaaattttcttctgaacaccactttccctgcatttcacacattttggtaagttgtactttcattttcattctaattaaaaatatttttgtaatttcttttgagatttattttttgacccatgtattatttagaagtgtgtttaatctccacatatctggggattttccagttatctttctgttatttatttctagtttaatcattgtagtctgagagcagacactgtatatgccttatttcttttaaatttgtcaaaatgtgtcttatggcccagaatgtggtctgtcttggtgaatgtttcttgtaaaatatttttcctgagaaaaatatgcattctgctgttgttggatgaattaggatataggtattaattatatgcagtttattggtggtgttattgagttcagttatatccttactgattttctgccttttggatctatttctgagacaaaagtattgaattctataaccatgatagtggattcatctgtttctacttgcagttctatcagtttttgcctcaaaccatttgatgctctgtttttaggcccttgcacattaagaattaacccttcttggagaattgactccttatcattatatcatctcttttttatccctgataactttccttactttgaagtctgctctgtctgagactaatatagctatttctccttttcttcctttgattagtatcaatattgtattttttctctatccatttacttttaatcattatatatctttatatttaaagtggatttcttgtatacaacatatagttgggtctcattttctgatctactctgacaatctctgtcttttttattggtgcatttaaaccattggtattcaaagtgattactgatatagttggatttatacctaccataatcattactgctattttttcccttgttctgtgcctttactcatattttgtcttccattcttcatctgtcttttgtgattttaactgagcattttatatgattccatttcttctcctttcttagtatatcaaccttttttttttcagttttttagtggttttcctagagtttactacctacatttatagctaattcaagtacactttcaaactacactatacacttcaaagatagtatcattaccttataataatataataatcctaattcttcccttccatcctttgtatcattgctgtcattcatttcatttgtacacataaacatatatattcaaatacaccattgctattgttattttggacaaactgttgtctgttaaatcaattacaaataagaaaaataaatgtttttattttaccttttttgaatgcagagcagagattctgacctatattatttccttcgtttttaaaaaatttatttattcatgagagacacagagagagagagagagagagagagagtgaagagaggcagagacacaggcagagggagaagcaggctccatgcagggagcctgacgtgggacttgatctggagtcatgccttgggccgaagtggtgctataccactgagccacctgggttgccctatttccttctttctaaaaaacttcttttaacttttcttgcaggcagatcttctaacaacaaattatcccaatatttgattatccaagaaagtctttatttctctttcacctttaaaaattaatttcagagcatgcagaattttgtgtttgttgtttctcttacactttaaagatatcactctactttcttcttacacatggtttctaaggagaagtgaaatatcattcttttttttgtttttctataggcaagtgttttttttttttttctctaggttcctttaggatttttttttttaatctttgattttctgcagtttaaggatgatatccttttttagaatttttctatttggtgttctctgagctgcctagatccatgatttgatatctgacattaatttggagaaactctcagttattattctttgaaatatttcttctgttcctttttttctcttctacttctggttttcccattacatgtatgtcacacctttgtaattgtcccacagtccttgggtattctgttctatttttttgtttttgattttggtgattcttttaatttatttttttcctgcttacttttcagttttcaaagattttattgatgtattctctagttcagagattctttcctcaatcatgtccagtctactaataaacccatcaaaggcattcttcatttctgtaagtgtttttaatctctagcatttctttttggtttttttgtaggagctccattactctgcttatattgcccatctgtcatgcatggtatctactttatacattagagctcttagcatattaatcagagttgttttaaattcccagtctgataattccaacatccttgccatatctggttctaatgcttactctgtcttgtcaaattttgttttttgctttttgatatgccttctaatttttctcaataaacataatgcactaggtaataaaaggaactgtaaataggcctttggtaatgtggtaatgagttatggggttgaggaagctttctagagagagccctatgattaggtctcagtcatttaatgaacctatacctctggacatgaacttcacaagagtttcctaggtttttttctccccttatgtggaacaagatggctacagtgggctattgttgggtacttctcttcccccactttgactcttgataataccccagtaggttaggctgtcactaaccagtatgccctgaagacaaacctttcttagaacagagtgctctggagtatttcaaaatggtttctttcttcctctctctgccagagcaagaggggaattttctccagtattttgtgtgggaatctagtcaagctcctggaggtaaatttcaaaataatgtggaacccctcctttggctggtttctcctgcagttcttaatgctcaaatttgcctacatggagcatccagcaattcctcaattataattgaggtttccctaccctgtcacaggttcctagcatgtttttcactcctgactctttataagttgagactccctgttattttcctgtctctccaatttggggacagcaatttgtgttatgtacacccctttcttaaggatcaaagaaaagttgttgatttgtcagtctattcagctttttgttagtagtagtgatttccaaagttcctaatatgtggaacctgaaaccagaagtcttatactttttattttaccaaataaaaatggaacctactaaattgataattttgtaacttgaatttttcaattatattataaacttgttctttaatggtaaacactttgatggagtttttattggtgaaagtatctttttcatagtgcatgtttttatctagtcctttattgcttagactgcttccaattttctgcattataaacagtacttctgtgaacattcatgaaaataaatctttgtgtacagtcatgatcatgtccttagggtaaataaaatagctcttcttaacctaatgccacagaagcattagcagaaaacatctagatactgctatagtagaataagttccatgtccttccacggtgattaaataaaattagtttccttgttctttcatcaattcacaaaatgatggagccagttctcacaatctctcattgtttggtgcttgcatgtgcatgtgtgtgtgcgaagggtggtggagggggggcacagagagagagagagagagagagagagagagagaaagacagagagagaaaatgaaactgcttttgaaagtagtttaaattattagagaaataaaggctatttttcatttcattttatagaagaatagtagttggaagaatctggaaatcccaggtatttttgtgcttttattctgcaacttttataagacagaagaaaaaaaaatacccactgtccttagatgtggcatggagatagctcttctgcagggggcatcttttttgttattcagctttactgatagaattacctatatgaaactggtaacttcattttatttttttagataaaatagcaaaattctagctgcttccatctatattgcctttgactcttcttgcttttcatcctgtcagtgtccttttgtgacacactaagaacacacttatacttgaaccatttggatttttttgctagccaatgaaggaaaggcacaccatagagaaccatgggccatctcagtaaaagtattagaaaagacttactataggatttaggtttattgttaagggatttgagggagtttttaagaaagcagggctttgctatagtttgcttgcttccaaaaagcagttgtaattttatgatctggtttattaatcacttttatccaaaatagggtgacaagctatctcacagtgcttcagactgaggggttcttgggacacaacatgttccctgcccaaaccaggaaaatccctggcaaactgaggggtaggttactctatctagagggcaaacaaaatgaaaagaggctaaggctgaaattggtaaagtagtagtagctactgatgggagctaggatttgggatgtttggttatttttgggattttggcaatgttcatatttttgcgtggattcatacatgactataaagtagtctcatctctgacttgatccatgatggtaacacagtggccatgcctgatgttgcttttactaggagaacaccaggccagctcccagtaacaccaagatttttacacagagtcagggcagtttgtagctgtcaggggctattttccttttttccagttctcttctttgtggatttgagggatacttttggagttgacctaagagtttactgtgtgatctagttgttagacttggtttcagccacattagtctaatgagatgcttcaagctttgaaacattgagttatgaaacaattatccatcacccttaatacaacttttttttcttcaatagataaaaatattttaaccattgagaccaagtgttaaattactaaatgttaaaacaaaaagaaatcaaatattctttccccttgagattcaaaattcacattagcatatttcactgctcttagtaatactgccataaagaatcctatgttatttgatgtagaacaaacatatttgcgcagaacatatgaacatttggatgaatattaatatctcactattaaaccagtgttgtatataagtactagtttatttagcaatgtttcccagttgtgctatttgtttttaaaatttggaaatatatttaaagagtaaataagtattttggtttaaaataaaataaattttcttaatggtggtagttgaaaagagatagtttcatctttttcaggtttgttaatttaatattaagaaaaatcttaaatggtagttggattttttgggagagaaattttaaactattaaatgaatatatatagttggtatagtttagtcactaaggtgactaaacttaatacaagttaaattctgaagcattatattaattattaacatccagctattttaaactcagtttgatcgtctcatattttaattaaattaattgtttatcaattagtctttataacaatattggaaacaatatgatcttagctatgttataatcatgatatagttcacattcacaaacttttctctaggttctgaggactaccttcaggtaaatattccaacagacctaggagtggaatgttccaaggaaataagtaagagaaaggaaattgaagaattatcaatagcatcaagaggcaccataccactggcagtggtgcttcctgctgaaattccatgtgaggtcagtaagataccttgttatttaagtggctggctctattgttttcataaatattgcttttcactagaatgtaagctctaacatagctgggccttttgtttactttcttcatggctctgtcctcagaatccagaacaatacctggcaactagtagacgtttgattaacatttgttgaataatttaatattggagaatatatgaaagcttaattttatcaggtattatttgacatgttgccaaaagatatatagttgctatacattatatatatataacatatatataatgtatatatatataacatatatataatgtatatatatatatatatcaagttactataaaaaagttggattcatattaaccagatcaaggattttatttttataaatggaaacattcctgagtagaatttgaaaatatgtatattgtggatcaccaatttttcaattcttaaacaggtagtaaaggcctacattattaagtggaaatatgagctagcagagctgtttcttttaggaataaaagtgtaatgtagcaggggatggagtgtgggtttctgagaagatgttatatgaaaacacatcttcctcctaagtcttcattgtaatgaactattaaataagctcgggcagagctggacctgaatctgggtctacataatctccagacaatagaaaaatgctggccctcagtgtctgtggtgttcagtggaacaggtcaaaaatacaggagagccttggaaattgcatgaaagggtccttctggtagaagccagtgaatttacgccactgaatgacaggggctgcaattgtctgtttgctaggaggtgtctttgggaaaagaatctcctgatcttgctggcactagcagtcctgggagaagacgagctttctttctgtgcagttatacaaaatttaattccttcatccccagaccccacgcctaagcagaagaatcattttttaagctattatggtatcaattccagagagataagaacttctttgacattcccttaaagagtccaaggaactaaactgttcaagtttcaggggaccgacacaaggtgaccaggcaaagaaaaattacgactcacttaaagacctccagaacttggggaatggtgggctaagcaatcagaactggtgtctcctattgagtcagaaacaatggaaaagacagaaataaatagaaaaaactgtaataaacaggaaatgagaattattctcaagtaggtaaaatctacacaaaaatgaaaaaatccctctgaaactaaacattactcaccaagaattctgcaatgagtctaaataactgctctgaacaaaacctacaaagtaaattttgaagaatatgaaactttccccactattcagtttgcaaagaaattgaaatgtaaaaattagaccccactaccctccaggctgagatagctcactcagatggttggataggatcaggattcttttaatagcagaaacttgcgctatcttttattgacatggatacaattctttcaaatccttggaagttaaattatttcaatttcatatctctcttgggtaccctgcttcttaaggaatcctgagttagaaggtcatacaagataattttagtgccagccacatgcactaaaacattcaaaataaactaaccgctggacaaaaaatagttcttccagtcaaaatcttgtattctcagaacatagatctagtacaatcttagtccatgtaacaatggccaagtaccagaggaaagatgtttgtgtccaaatgaatgaaaatcatgtttcagatttgccaggataactctcagcataggtcctcctatgctgaataggagaataggtcctcccctcttcctgagacttgagacttcaccaccatcttacaaaatctagaaatttagcatactaacattgcgagtcaccaggtcttatttgacacatgcctctttcataactagtacctttgaaatctagtaagtttccttacagttttgtgtttccaagatgccaccactgagttgggtaagagtcttaaatagtagcatgaccgtcatcctttctgattaagggggagttaaggtctctcctgctgctgtttcttgttataaaattgcttgggtctggagaatttccctccctattccctcttctgttctggagaatcagctttagtagatttgttcaactaagtcagcaggttatgttgctactgctttgacccaggacccaggagcatggaagtcttgatccttctgcatattcatctttctcatcatttttcctttccatggattttaggtttcttagaccttgaaaatcttgtgccactttaaaaatagctacaactttaatgcagacctaattatgtagccaacaaaactagagcaactcttagactttatgaaggggtatgtctctgaagggcccatggaaaagtaacaagaatcttagagattacctcctacccattcattccttggacccagatatagtttaaaataatatacagaaaactaacagcactgcccacttatttctcataccaaatcacagtagttcttggtaaccaaccttaccttaaagtgtgatctcgtgacttcactgaaaaatacaggaaaagctacagaatattattgtgtttaccagtgcttgaagtgtctctgtggtgaagtgctgcccctttggtggtaaggttttacagtgaccaaaagatatcttttccttaaaccaagtggctctcaaactaagctgcacagagggactattagggaaggtttaaaataataatgcctgaatcctgtccccaaagattcagatataaatggtttgggttatggcctgaacattggcagttttaaaaacttcccaggtgatcctaaaggtgagaattaacagccaaatttggaactattgccataacctaatcctcactgtgctgttgtgggtcctatagaggttagctggaggaagctgtacagtcctggcttaataaagcccatgagactatgcttgctaacctagtgcatattaaagcagttataaatatatgaaaagagccattggcctttggtatacaaaagtcatgatgatgcactgggataaaaattctttgttgactgtgcataacatatccttaaagcctaaagaaaacaaccctatcaaaaccagaaacttcaccatctatatgttatattacagaccaagttgtagccattttgtttaggtcctaaggtttgtgaccaatgccacaaaactttaggccttggaagtgatgattatcagaattctttctgtctctggatgttcagaacaaaatagggataaacagatgagtcagaaagattgagagcaaaatagcagcttcataactgataaaaccaaatttatgatcatttgagtgtcttagtactttctccattgaattaagtgtacccatataattatatttttaattaagatgttcataatagttaaggacaggctggaccttccctcctagaacaggccgcttcctccatggagatgcagaaaacagatgaagctgtgagtcctcaggggtgtcatgagtagagctgggcatgttcaatttcaaaatgtcatattttaacagacttattccatcttaattttaaaaatatgtacagattgtctcttatgtacataattcttacaagatgccacaatgatataaaaaaataaaaatactttaggatgttgaaaatgtagctgggaagccaagacgtagacatgaagtattaattcagagcagagggcataatttagaaaaggtcaaatgaggagtatgggtgtgaaatgctccatgattcctttggaagttattataggcagaagttgtagggaagatttatggagcttgtgaaacctgagttggacgttaatgaagaggataagtgtggggagatggaggggcagtaggaactaaatagcatgccagcattatgtacaaacagagctggtgcaaaatgaaaatataggggctctccttcaaattagtaaggactttaaggcagtgacagcggaacatttaaccgagtacaggacctcccaagtgcagggcctgtgcaaatgcacggctcataagcccatgaagctggtcctggatacaaatgaagatgtaagtgcctcttttggggtggccaatatgcttgccttttgtttaaaactattctttggagatggttgaaagttggagtcctgtcctctattaaggagagaatctttttttttttttttaagatttatttatttattaatgagagacacacacacagagagagagagaggcagagacataggcagagggagggctcctcgcagggagcccgatgtgggacttgatcccggattccgggatcaggccctgagtcgaaggcagacgctcagagccacccggtcgtccctaaggagggaatctgatgcagacgcagcaagcttgtgtgagcactcatgaatgagaatgagttagctgggtgaataaaacattggaataggttgcttaggtttactagccttggaagcagagaggtcaaaattaaacaagacgtagtgtacccatgaaagagagctctgaaaactttgaagcagaggatcttggggatctgggccaaagtaaatgggaagtcattacagatttgcagtaaggggacagatgtgagcaacaaaggaactaaaagagatagacaggaggccctgtgaagaggaagtggcagatgtgaggaagaggagagcttcatgttcctgatgtcaggagtcacatgaagcagtggcttggtgaggcatacagaagaaagcagaggggcattcttgacatgtgggaaggtgtgttagctggaatagtggcatcttttctccatttgcaatgtaattggtaaggagaaatttaattaaaatatcctacagacatacttgggagcattagacgctggtggaaaaatgcttgaagtggaagtgtgtcagctgaaccaattcagtgacatcaacgtatttgtagaaactctttggtgcagagcagtaaagatacatgttttatctgtaggtggtttatgcttagagagataatttttaaataagagaactttgaagttaatgacttttgggtaaacacagtttttataggagtgtataaatgaggttgctgtttattttattttgctgaaatttagtttgactttgaagaaagcatttgggaaggcgatatctgaggtaaacataaaaatggtgtttggttttggttattttgaggggttaaacagcacaatgtagcatacagattttgaatgagatagttctgattaatcttcagaatgtaactctgtattagctaccatgttcttcaaagagaaaactaatagccttccattttggcttcagagtatgtatgttttggttaggattaaaggaagtacctttatattaaaagtatttgggatccctgggtagctcagtggttgagcatctaccttctgcccagggcgtgatcctggagtcccgggatcgagtcccacatcaggctccctgcatgaagcctgcttctccctctgcctatgtctctctctgtgtctctatgaataaataaataaaatattaaaaaaataaaataaaataaaagtatttaacataattcctagtataaaagaggtgctcaacaaatatttattccttcttttttttctccatattaatgccaatctttttatccctaattatttttttttggatttttttcaaaagaagttttcaataatcctttttgccttatatacgtagatttagctttttgatctccataatatcttgaaacttttagatttgtaacctttagccaaccttcacagatactcatcacaaataaagtataaccagaaaagcccaattaagtaagatttaaaactattggacatttaaaacattggacattaaaactcatattataaatattaagatgtcttaaaggtatacttatttaatcctctcatttatagataaatgtaccaagttctagaaaggtgaaataaaaggatttggggggaaaaaggatttggttgcaaattacagctccctggaatctgtgaagtgatgctcattactctcagatgtggagcctcacaaacatggaaaaagtgtgtcctcactgttttttcagttgcaaaagatagggttggagaaaaaaaatgagtactcaacagacaccaagttgagaaatttaagatgtagtgagaatcatatttacgtattttatttctgaatatggttgaaaatatggacatgtagaaataattatcatgtgcaaactttgtcattaataaatataatgtgtgttttagttaatgtaatatttttcaaggccctaattgggtaatattttttatcttatagaatcctggtgaaatatttattatcttgagagatgaagtcattggtgataccgtggaggttgaatttacatcaaatagtaaatgtattaggatgcagccaacccgttggaataagacagtctggtgtatgaaagctttaggtaagaaacttttttatgcattaataaatataaaacatcatcctgagtttctttcaataaatttatgataaatggtttcacttctttacagaaactaatattgttctgcaggtgcttcctgtgcataggtttcacatacctgaggtggttattagtaatctaggttcccaaaagtatgaatgtgtaaattatgagaaattagcttgtgtaccatgtggtgaaatgactgcctgcaaaattacatcgttttaaatatgtcagcagagatcactttaatatatgtcagctattaaatatggaactaaaattatttactttattgtaataggattaatgaacatttagactttcattcacgaactttttcctttcctttttgttttccatgcctgtgaatatatttttgatgctttggaaagcatatttctttttcactatttatcatcatggtaccatttccttatacagtcttccctcttttccttatttaatttatgaggttaaccattttattatccacctttgcttgcagcgtgataaattgtatcagatttaaaattccaggtggaatagccacacctgccacaatagttgtactaaaatcagtctattctattaatttctgaatgttttgctataaatgtaggactacttggtttttgaatttatgatattttgcaatcagagaacaaaattaccttttataaaacttcacaaatgtgacatttaaaaatacattacaataaaaagtaaacaatattattatgaatctataaatataatacaaacatttaaaattaggggcacctgggtggctcagtggttgagcatctgcctttggctcaggtcatgaccctagggttctgtgatcaagtactacatcaggctccctacagggagcctgcttctccctctgcagatgtctctgcctctctctcttgtatctcatgaataaataaatgaaatcttttttaaaagacatttaaaattaaatactatgtcaaatgcatactaaactttttttctttttgaatacagctatatgaagcccatttatagaacatttagacatttctattgtgttatgaaaaccatatgtctcctctatgggcttccttgcacaggttaggtcagacttgaattatagaaaaggaaagactgtacactaagcattcataaacatttcagacactggtccctatcatggatgaagatatgcagaagtgagatggaaagaacattgtctttttctttctcttccaaaaccccttactcgtgccacattttataatgatttaatgaacattaatttctaactaattagttttcaagtttaaactaaatctagttagactaaaattgctacagaaaaatcaatattgcaaattaagaagtacttagataatatttcccaaacagcaaatcaaagtgtcaaataaaattattcacgttaagattatatgttcagtttacctagcagctaagagcaatgcaaagaatcaattatgtcaaaattcaaatgtttttcagaaaagcagaaaactaatagtctatatcttttaagatataccagaccagggatgcctgggtggctcagtggttaagcatctgcctttggctcaaggtgtgatcctggggtcctaagatcaagtcccacattgggatccccacagagaatctgcttctccctctgcctatgtctctgcctctctctgtgtctctcatgaataaataaataaaatcttaaaaaaaaaaagatataccaggtcatatattttctatgtgattgaacagcaaatttgtaataagaaataaatagaacactatttaccagttatgctgtttaaccttcccaactcatattttctggaaattaaagagaaattttcttaccaaacgtagaaatacatacgatggttccatgaatgtgtgtgggaagatggatgggatgtacctccacagaaatgcccccagagtcttaattttttttttttttttgcctcatgccagtgtttcttgacatttttttaaccctggtaagaactacattttatatcaaaactcagaacatactcagaaatatataaataaagcaaaagtttgaaaagtagtcattaacagttttagccatgatatgtgaagaacttaggatttgtcactcctattcctgcaacaagataaagctgaacaagctgaaaatcagtgactatccttgaactcctcagagaactaaggttgcagggcaaaccaccatcccaaaatcttgagaggaaaagaaatcaagagagtgacatccaagatcgccttggctgggacggaagtcactggagccatagagaggtagcacacttaaatgggaattttaatgaattgctcaggtggagagtggactagctcgagtcagaaattcctgagagcacccataattttgtgggattcacctccatgaaccctaccaggttcacatagtgaagatctgaggaagatcgtctagaggttctgctgggttagaggaagaataatcacggtgaaatactcccagtgtactctccaaacaaaagcctactcttccagggaagccagtttatagtgccttatctcagctggggaaaggcatctaccccatcgtagcaccctctaacttttctgtctcacttagaatgtgggaggaaggtatgccacaggagaaacacttgaaggtgacagcccagagacaccagtccacttgaagtccaagacttaatcacaatattagaatgcttccccccaccgcaacctgaccaccaccacaaaacagtttcaatataaatggctgtaggttatagttggaagcactaaaagatgcagaatctttctgaggaggaatagttaaggaagcacaagagcaagaggaaagacaaaaacaaagatagtagagaaatttgaagcccctgggcacctatggctatattagacatggactatagcccaccgcccagtctgattactataaattttcccactacgtggctacctactttagttcttatacctgacacagtatgtctggttttcaacaagaaattacaggagatgccaaaaagcaaaaatagtctaaagaaatatcaggccagacttggatgtgacacagatgttggaagtatcagacaggaaattttaaataactatgctaatatttaaagactataatggaaaagtaaacaatatgtacgaatatatggataagtagagagaaattccatttcttttcattttcttcagtgctagtcaaaaccaactaaattgataacatgatgggtcacaaatcatagatggagaaactgctttgtagctatgctgaccaatatggtggccactaaccaccgctagttatttaaactttattataattaaatgaaatttaaaattaagcccctcagttgcactaaccatatttcaagtgcttcattagaacatatccatcctcttggaaagttccattgaacagcagtgaacagcacagtatagtaaagttccttttcttctatatcaattgtggaataaaagagtgtccagcatgctctttaaaggctaataatttttccagagtctttagtgtagccaaagaaacaattaactagtggccaaaagagctgcttttattgttttgagccatttactgggtactgtagacaagtcccagttgtgcttcaccttctttattgacaaaataacttctctaattgcatttcaagtagataagagaacaaaatgcaaagacaacacttaaacatccaataaaggaataaagtgttagtccttccttccttctagcaattattttaaacataagaagtggcttttctggaatatctcaccctcagtttctagatggggtgtaataaaccatttgaaaatttaggggaaccatctttctaagtattgtattagaatataacatcctgccttttcctactcctttgtttatatctattgatatgaaaaccaccaaatttgtttatcccccttcacccttggggaatcatgttcaacagagctcctaatacatgttatctcatataaaatccctgaactcattggtgatttccaggggcaggaaagatctcctctgtctcccacacctctaataagagtctatataaacttaagtctctaaaaaaaattttctgtacttctttgaataccaagttaagatggagggatctaaactaaaatggaaacataccagagatccttccgtattaggatgcatcaagttcagaagatggcctccatcttgccagtatctttctataagtgtagaaatttcctcatataatttgatttctcatacctggatcatgatttaatcagccatgttacttaaaatgtgatttgctctctgtaaatattctcttgtgtgatactaagagcttcatgtagttgaaataagccatggtggaaatatatacatcacagaaattggcaaaaactgtaagtcaggactttctgattttcttttgggagagtcagtttatcaacacaatactgggataaagaagacccccattaaattgagaaggtgtctgtaagacccatttgctgatatcccattaatcagaacttaatcccaagatcacctctagctgaaaggaagaacagaaaatctattctttttagctggataacaatgtgccctgaaaaaaaaaaatcaggattctatttctaaaaggaagagaatagattttgggataggcaattagcattctcttttagaaaatggtacctcttaaaactaactattattccttactattctttcttttctttggatactttctcttagtcgttttaagtgtgtgtgtgtgtgaatactctcaacattgaggcatgtcatatactctgagacctacttacagtgcccagaactctcagatttttcaccatccaacccaactctttgccatattaatattctttctaaatgtatgtttctttctaattaggtaataattaagaaaaaaattatgttaaagtacagttatgccaaccagtgaagaaatcttaagatgctctttttctaaatggtatctttcctttctgatacacttccatatcttaacctgatttttttaaagaaactatcctctaattttatcttcttttgcctctcagcttaacaaattcatacactcataattacttcattttctaaaaaacacacttttctaatggaatctacaggtttgtcactgacaggaactgtcctgtgctaaacaaatttgaagttactcaggaatctgacgtagtctcaccttatgccaaaaattgtaaaaatacatgtgaagtctctaaactagaaatttttttgtgaagattttatttatttatttattcatgagaaacacagagaaagagaggcaaagacacaggcagagggagaagcaggctccatgcagggagctcgacgtgagacttgatcccgggtctccaggaccaggacctgtggctgaagcggcgctaaatcgctgacccacccgggctgccctctaaactagaaatattaactgttgtttacccagaaggaagagggtattcctctggtacctgcagctcagccagaaaaatactttacacagcagggggataaccaataaagagttgaaaggaatgtaaaaatatccacaaaaatatagaatctctttgttaattaggaagctgttctgaataatataaattataatttcatattttattgaaagggcatgaaaaatacttttagggcatgcccagtactatacttaacctgagcagaaaaataacaattgataagaaatttttctaaatatcatatttgagaagggcaattttaatatgtactaaattgatatcagtttttaggcatgaccaagcttttgaaaccaaaatatttatttcataataatgttctaatcaaaagaaaaccatattgatttcaatttccatggaaacattagatgggttcatataaggtttaccacatgataaagggctaaaattggttcataaaagaacacagcaaaaatagataaataaacaaacaaacaaacaacaaacaaataaataaatgcatttagggaatataaataatagtgaaagggaatagaagggaagggagaagaaatgggtaggaaatatcagaaagggagacagaacataaagactcctaactctgggaaatgaactaggggtggtggaaggggaggagggcacggggtgggggtgaatgggtgacgggcactgaggggggcacttgatgggatgagcactaggtgttattctgtatgttggcaaattgaacaccaataaaaaataagtttattattaaaaataaataaataaataaataaataaataaataaataaataaagaacacaacttcacagacaatggaaataaagaaagcaaacagctcctcagttattggatttgtgaaatatgtgtttttaaggggttagtgaatttagctctgactattcaaaaacacttaagattatattttctgtgaactgtttcagtttgcccctcatctggaaggacaaatcacttaaatcccagggacagtgcaggttgacaaagagtcttaaatggcctgaaccttgagacgttaaatttgaaaagaattcccttttattgttgctcctctgttagtttgctttcttgagtatctgtgtttgttgtgggcaaggagttctatcttcttgcactataagcctttttttcctctccataacttacaccaataccctcacttggtttggttatcttgacccacatttatgtgtacttccagtgttaaatgttgcttcccaactttagatagtaaatagcagaattcactttccctctcttctgagtctttaaattaattggcttttgtttttattaaagagctataaaatgtagatatcgtaatgaccacactcaaaataaaataatgctcagatggcacagggttctgtttatgaaaaggccaatccaataatatatatttaaggagggcctcccagccagatgaatccatacggaatcctctaaggtcttgatttaatactgacattcaatagataaaaaatatatatagtataaaagtacttcaaaggacacctcttgggaagattattcaaaaatcaacatcattatcaatagagctgatgatacactatatcaagaataaaacagttcatctgctttcatgttagattttaaagagttagaatatttttttaagtattctatatcaaagaactaccaagcaaatcttctcatcatatatttctcatgattcatcacctgactttctttcagaaggtgaacacctcattgcaaactctaatctctttcctgataaataaaatcaatatcttctttcaaaattgtctttctgaaatctaccaaaattgatttcatttgaacttagtaactctagagctctctttaccatatgtgggcagcttcagctgatgagatgcacttttgttgcagtttctctttcattaactgctgggcagatgctgactttgggaagagagaattttgatatatattttttctttataacacaggttacgtgtctagtcaagcaatatcgaaagagaaaattacaacttcttgatctacttaagtttaagattagttttgttggctttaaatctctctgagcaaaattataagagggttctttaatctcctcaggagataaagtaaaattaatacttaaaccttagaaacatttaaatatttaaagcttcatcgggtgcctaaggttagaacagactcttttggtcttccacttatcagcttatattccaaacaccagatgtctgggtgacaattttccaggagaatttgggaaatatttccatgacagagtgtcagattttttaaagtatgttctgcaaatggatttcaaggcttaattttgtttacttttgttacaaacaaaacaaaataactttcttactgaaatgattgttgttattctttcataacaaacccaacttttgagagaactctagccctgttgatatgttaatgcagaattcacaaggcatcatatcagtcactttgaacaacttgttcacaaaaaaggttttgataaatgtatagtataaatagtacttaaatagcagggtattaaaccttgccttcctaagagtttttcactgtagccaattacataaaagttcagtaatttcaatcaattctaccttttctttccaagtacaattttgagaagcttgcctataattaacaagaatttccttcatacgattattaacagatacacaactttaaatgaatgattgtatcattctaaattctaaagtattatgtaaagataagtatagattactccaaaaaatgaagtttagtagtagggagaaaggctttagggctttgcttttactgtaatcttagcatgttctgatcctgtaaggttttatttaagtaagttgaaaattgtacattattttttgtggataccaagataaaatgaaacgtttagttctagaagtaagcaagatcgaaatgaattcttaaatattccttcatattctctaaaaggtataataataggatacatgcattatatactcagctcctaactcttctttgcattttacaagactttcctgctggctctgtcaatgtccatgtctactgcgatggagtcattaaggccacagcagagattaaatactctacaacaaaggctgtggaatacctactgggggtagcccgtcctggagatggcgtgtgccaggtaagttgatctttccacaaagttgatgacaagccaaagagccaatgctggataacagaatgatgctttggtataaagtctggatgttggttatatgaatgaattgtggatcaatagggagggaggaaactgacttctgtggtgcagaggtgcagagatacagatgaattagtttacttacctcaagtcattccagagaatgtcactgggaaacaggttctccatttcaaaatcgcttttgggttgggctcattttaaagctgtcacagagcagatagagaagatagaaagattaaggagaactctttttttttttttttttttttttaagaactcttaagaaacgaatacctctgtaaccagatctggacaatgcaaaagaaaagtcagatcatctgatgctttgaggttcaggagtcagacaaaccaagaatcctaccaaaaagggaacataaggcattaatcgaggaattatcattttcatgtggaaatttccaagcttggtgtgtctttcttagtttcatttattactcacctgagagtgaaaattattgctataacttgatagtgtttattaggaattccagaactgctcagattttcagagggatttttcaagccccgagttcttttgccagtgcgacaaggcattactcaacaaggaggacttggtgaggagtttctagtgttctcaaaagacccttctttgagacgtttatcagtaaatgcagatgctgccaatgccctcctgggtatagaatataacaattgcattttgattcatcttaagcaattggtttaatttctgcattttattttgatttttttgttacctgacccactttcaataaagagaaagcttgtttaatgtggaacatttatcctttggatgtgcttattccattgcattttcaaagtgaaataaatggcatttctttttatagctacctgatggttcaattctacccttgcctggggtttttggcagtggagggggagggtaaaagggagtaaaggcgaaaagaggtagctgaagaatgagatcttcaaatttaccacaaaacactctttaacaaaaccatcaatggtactgtggctgataggttgtcatgaagccttatctgtttaaactagtttggaattggacgctttgttatttattttgtgtgacacatttttctattagaacaatggcaaaggtaaaaatgtcttagagaaggttcagggaataaaatatcatttgtcaaacttgtgagtctgaggacatttgataaaatgtctatattgtaattgcaaagcaaaagcaaaagtaacagcttgaaatacggattgtttcattttattgtgacaagtctaccttatcttccactttaaaaattccattcactgcaaaaagattaataaaagtgatatattggattatcagtattcagtcagcacagaagccccccaacacatttttgtgttgcttttgcctttggggagaaacaactttttatatgtaataagattaaatataatgtatgactcttggacaatgtttcagagtgctttattatgccaagtcatgatttcttctacagctgtgagtgaataaaaactgttttgcccagttttaagataactagacaacactattcccctttcaccctaggcatcagaaatgagaggtgagaaagggttgttatgattacctcttaggattctcctcttcccctctccctgatggcctagtgaaaagaaggatgctgaatggtcacagctggatgcaggcttttactatcaggagagagtcactctgagcctgagggagtgctaagacttagaattattctaacttggtgacagcagaccacagcatggtaagaagagacactaggagaagtagtttgatctatagaacttcagataggcttaggagtcctgctactgtcgtcaggggacctagacgtgtagtgaagttagatcaaaatctgaggctgagatctcagacgagacccagcagtactctcacgggcctggcggaagaagcaggcagcaattcagtcagcctccatcaagtcttagaccaggaatgacacgggatggttctctagtccactggcatttggaccacatgagttatccctctggtgaatttgtagagataatgcaaggcaaactctacccttgatgaaaaagaaacagctgcaggggcatttccttagtgtgttttagggttgtttggcctcagaggagggggcccaggctacactatgcttctccttgcaagtatttggatttcccatttgacatttggctttgttcccaagtagattatgtttttaaaaatgtgcaaaaagctttgcttttccgttccagcatagagcatttacagtcattgtttatttcgtgtgcttgctactgtcacttatttgggaaatttggatattttgcaaatagtccgctgaacatctctattcaaagaacagcatgcattgtaattttattcactgtggttactgtgtatttgtaagaacatacaccagcagatttcaatttttttcagtatctcacctacttcactaaatggcgtattgtaagagccatcctgggttttcttgtaaattttcatggcagatgtgatttaagtaatctgaaaaacttagtagtaagatacttcctataatcatttaaaagctattggcgtataataatgtgaacactctgtgagtttgaggaaagtagtactaatgagaaatctgtgggttttatttttcagtaatcctagaatttgtaataagagactttatgttatcatttaaaagctgttgggcatataacaaaatgacaattctgtgcatttgagaaaagaaatgatgagaaatttgattttcttagtttatttttaatgatgtccattaggttttaaatagtttacctaaatcagcttaattatctaactgcttcctaaattatttcatctttttggggtagatattaaatcccagcatgtagcctcataaggtacaatttccagagcattacctaaaacaacaaatttaagagagattgagtgaaaaaatgaagaaaattgaaagcccaacttgggaagataattagaatgcaagtgctagtgttagaaatactagtaactcccactttactgagataattaaaacctttgagatgttcatcctatttacaaaagtgtctggggacaaccataaagttaactttgtataggaacaactagatacaatctaagaagcaaattaggtcaaaatgctaaaagaacatagcaagtattgatcaaagcattagcatcctatttgttaaaagaactttgaggtagtaccaaataattcttcttagctgtatttcttggtgaaatgatatctttctgttgcatatactgatcattgtgcatggtttgctttatactcattggaatcttattttcatcttcatgttaggtacacttcatttccaaattttattaaattgtatatatgtttccatctttggtttgctgcctgcaaatatttaatacagtatgaacccagctacagttgggttgagggaagatacaggcgtaggaaagtctcgtattgtgagggacttcataattgtggatagttctcttaaaggttagtttttttcacaaggttacattcagatgttagcaagtgtctcccttaaagaattcttcaggagattcttccaaaagcctttaattaattttttaggtccttgtctcctgggaaagaattggtgtataccatcctccttgttttcattagcttctgttgatgagaggtgcaaatgcagctagtttttaaactatacttcaccataatgccagtgttagctaagacactctcttgctacagccttctagtgactcactggaaaaggtccagactcctttgaatggtataccaggctgcagagtcactaatctttcccagcattggctcttaaccgtagcattctctccgtgtcttcttaattctctccatgattcctatgttttagccctccaggatttctcactcttgaacatattctgctcttccttccctccaccttctttaaggcatggacattgtgtttttcatctttgcacatgcagctactttcacataattagctcctgacatgggagacaccatgaaacaaaaatgggaaaataggagtgcttgctttgtcctccttgaaaacataagtgatcttagaaaacataaatggagaactataatatagttatataaatgataagaaggaaaatgaaaactgccctcttaaaatacacttatttatttcccaaattgaataaatgaatctgatacttatgaaaatgaagaatgtttggtggccctggataccagagaagtgaagaatttgtgctataaacttttcatgttctacctgagtaatgaaccatatgttcatcatgtagctcattacagactttgaacaggtatatggtttcagattccagtctgtcgttctttgagtattaagaaaatgattactactcaaaaaatacctaagatctgtctctttccaaaggctgcatactatctctcctttccctttcctggaaagtgatggaagtgaaagtgatagtccaggagaagtagctctggatgggcgggagcaagagttagattctttaaccgaatcagcaatcactacatgagactttattctccttagtgtgaatggaatttcctcatgtgtacagtgcaggaagctagaatatatgctctctaaggtcacttctgactcaaataatccacaattttatgccaagaaaaaaaatacttcagttggggattatgaccactctcaccaaagaggaatcattggcaagtatgtgggttctccatgcagcctttattttacttgtattagcacttcttcccagttctcctttcagataaggacatgatgtagatttcatgtacctttcatcatttttattacttgtttcctatttctgatgaaatatttgaaatgaggaggggaattaaaaaataaaatgcaatttggacatttatttctggattcaatttccatagtttatgctcctagttaaaatggatcagtaagaataaggcatatctattaaagcttgtgttttactgataaaataagtgacattttgtgccatgtctctacatgtggtaatgattcagcaatggttaaggatgccttattgtgttagctcagacttaaacaatagtactcagctcagttaccaattattgagtcaaacagaaacataccactgagtacctacgtccacttatttaggaaaaataaattagttaattttaacagcagatatagcagccattgtggggggggggaataaatatttctgagtgcagttacttggttattaacaaccatattttaagttcctgttaattctctatctcataagccatctcattccatattggctccattcatcttagcttatgtttaactattgtttagaatatattttattaatgacactctaatttatgtctgcttactcagtggaaataaatactccatccatttttaatttctcctaaactaagtatctcttttgctttttacaatcccctctcctcttgtcactggggtggctttgactgccctttgcagaggatgtctgtgtgcccatgtgagtgctacctgaaaccaaccccattcaagtgcagccttctgctcttcctgtctagctctttagaggctacagatcttctttaaccataatgtttttaaacaattcagaaaacaaagcacaagtcaaatacagttaacagtgttcctccatagctattattcataacaagataattagagaaaattagagaatctgcctctggaatgaatctcaaatcatcagtaaccctttggaaattagaagactgaaaaatccttatggcaaaaatgtgtttgttggcattttttaaaaagtcattaagcattctgtgggcctctttactaatagttatagtatcaacagtaagataaagaaataaagtaaatatataatctgggaactctaaattgtgtttagcattcacgaactatgcctctacagtaaacagcatcaagacgcaggaaatgaagtaaaagaaatctttgacaaggaaaattcaacttctaagaatcctgccttccctctctttcccccaccatcgcctgggtcagccatttagtcatcaacctcattacccaaagtagcagcacacaaagatagtacttccaatttgctcaagtcccacacagcaaatccagtggatatttttcttattcaatttaacttgactttcagcagcagctaacacacaccattgaccaaactcactttcctgattttttccccctatctttcatgaagccatctctttctggttttcctctacttccttagctttccttctccaaatcctttgtaatctcttttttctgtgatctagaaaaaaaaagtttccatttcaatgttttggttcttagactctcttcctttcttgataaatattcccttggtagggtagctcattaacttgtaagatatcatccaaatgtggtttttccttgaaggcccactctttctgccttgagataggcatcttcatcaatccagttcaaacaccagaaacagaagaatctccttaaaacctgttgtcttacccaataaattcaatccatcattaagccatgtcagttttactcactaaatattgctcacttctcttcacatccctccagttctaacatccccatcctctgcttttcttcatccctaaactacagcgggctcctgcctggtcccccgccatgtgttcttgcacatgacctacttacttatatcagatccatagtctgaggagttctgtttaaaaaaaagagatctcagcaggcaatgctcctgctcaaaccctttgatagtttccagttgtgtggatttaaaaaaggaaatctttaacatgaacaatatggccctgtagacagtgtcccctctctttttctccagctagtctcactcccctttctctccctctgcactccagacacactagcctagatggctagttcctaatctcctaccacagggcctttatatatgctcctctttctaactgaaatattcttccaatctcctttcctctcactgtcttcccctagttaattccagttcatcctccaaattttaattacagcttctcttcctcagtgaagactctcttgaccttgtacatctccctgtgtatctctttgtagcactgaacacattctatgtgtgtgatatttgtcaaattaatatctgtctcttgctagactccaaagtcatttagggcagttactgtttatttgttcccctctttcatctccagagccaagtaccctgccagacacatagctgatgaatacctgttttgttgggccaaagaatgagagggtgaaagtattttatacattaagttaccctcccaacctcaagtaacctggctatcagagccccttgtcccaacagacaccagtttccccatagtacaggcacaaccctttttcatggcttagtatattggactgttggctgaaggagtgctgtttctgactgaagtagaaagagaaaggcaaagaagggaaagaaagacaaaggaaaaaatcaagtgagtataaaacaatgagactaattctaactatcttgggaacgagtacctaaaacaactcaaaaaataatttcaaaaacttaggaaatcatggcatcatttaaagacatcttttaaaaaattttatttacttattcataagagacacagagaaagagaggcagagacacaggcagagggagaagcaggctccatacagggaggtgggactcgatcccgggtccccagaatcacaccctgggctgaaggcaggcactaaaccactgagccacccagcctgcccttaaagaagtcttttttgaaagacatttctctgaatatattattgcaattttgttttaaaatttatcttatgttattgcaattttgttttaaaatttatcttatgacatatttgacagtaagttgttacaaaatgattgccaaacagaagtagaggtgagaaaattcatggttcttatgaccactctttctaatgccaacaataggagaactctggttttttgtttcattattaatgtcattaaatatgcctattaatgagtctgttcaagtttggttaggttaaagctttgagattttttgacattatgtaaacagcaggtcaaattgaaaaatttagcatgatcaagcttccatcagcttctaggaaaattgttcttgtttttaaaaaattctattagattgcctttacctaaattgtgatgtttatgcatgactaatggagatggcagaaacttctatttcttccccaaaaattcttttttcctttattatatgataacataacttctatctaggcatatgcttgccagggtaaatattgcacttcctgactcccttggaactaggcacaaccatctaagtaaccaatggctggaagcatgtcagtagaagtgatgtgtgcacatctctgatcatgtccttcaaaggagggtagagtctctccttcactgacttttttctctgactgatcacagtgacaacaaaggagtgatggatgaaccatctaggaccatgaagattaagtatagcagagaaccatatagagcctgcatcccttaatcagccccagatggcctaattctaggctttcatgttaaagagaaataggcttccatcttgtttaaaccactggcattttgagtctcagtactacagtatcaaaacctacattctaattgatttacaagatgtgccatttaatttaagaatttagaaagaaaaaacaaactattttctttggttgctacaaaagccagtttggggagctgtctcctccttagagacttgtgtgccattttaaatcatagtgattatatttcatgagtagttatgagagctcacattatagtatttagagaaaaagggaatgaatatatactggagattttacacatttacttatttaaattcttaaataattttatcagttaagtattattaaggtccatagctaatgaatcctggagcaggaatttcaatacaggtttctcagatctcaaaactttttttttcttattataatatcattttcatgttatataaagagattccacagctctgtgaatactgaaccaataataaatacaacatttttgaaaggagtcttaaactgaattatttcatattttcgtatttacttttgcatttgtaatcatgttgaaacgactaaaaccttactcaaaacttagaaaaattattttgacaaggaaaaatatctcctttaactaccctattcacttatttagattactcctttaactgcttggggttaaatactcaaaagttataaaaggggtgggggctttgtaaaacccagcaagtatttgctttgcttctttacctgccattgtctttacaagctgcagtaggaaattttgtttaagatgtattatgtaagctgtaagattcccctttaaatggctattttgactcacttttacagcacacattatgaacaatggaacaaataaagtgacctttcttttactagttaaacttaaataagcgtaacagtttattataataaaaagaacatgttttcagctcaaccattgactttccttttggatatctgaagcaaagagtaaaatgtaaccattcaatggaccttgtctcctataatattttacatacctcctatggttatttcacttaagtggtttttcttgctaattaaggtactttactattactgtctttctctgccatatctataaattgcttcgatatcctgtggccaaccttataggtttcttcacccaccgtcaatgcttaaaatccctcttttttgcccctcttggtgtggaagccagacaagccagattctcacattttcaaccactccatgcagctagcaattgtcatgtaagacagttttagacaatgagacacaagcggacatcatcagagctttggcttttgtagttaatagatgtggttgacagtagcctacattaccatcttccttccatctatgtgccttccttccactttctgactagacatgtgatgtttgaactgcagtgtccatcatgaaactatgggatcataatttattattcacccaaaaactacatcaaattttattctttccactagggctacaatgtagtattaagtacattgtgtttagaatgtaaaagcacaagttaaaatccatgatgtaaaaataaaataaaataaaataaaataaaataaaataaaataaataaaataaaataataaaataaaataaaataaaataaaataaaataaaataaaataaaataaaataaaataaaataaaatccttgatgtggcacttactagctctacagtttctggtgttattaagttttttgtaccatagttattgcatctataaaaaaaaaaaggatttaaaaaaatttaccatgcaggaatgttataaaaattagagacaaattatgtaaagcctaccacattctaggtgcccaacgtttagttattattattacctacactctttgagtctgtttcactgtataaacttatcctatacctgcaacatacagcctttcatttgagtccaaatatctgaagtatctcctcactgcttttactatatgatattgttctaaaatcctaataagcgtagtcttttaatgcagtcatagccaagcagtgttcatgccccttattcaagaagggtaaattagactttgaagggatgttgctatgtaaatagatcttcatctcaaaggattcagctttgtgtcatgaatatttcctggtttgtttttatttatttttctggttcatttttttcctggtttgtttgtatttatttgcatttttaattaacagaaattgtagatgcatatgtatttgaagaaataatagagattgtgtttggttttggtttgtgtttgatttgtgttttccaagaaattgatccatttcatctaacttgtcatacttatgtgtgtagagttgttaatagtatgtctttattatccttttggtctgcagggtctatagtgatagctcatgtttcattcatgacatttgtcatttctatcttctctttttcctttagtcttcctagaggtttgtcaattttacggatcttttaaaaagaattatctttttgttacactgactcttttctatttacaattcaactgatttctgtattatatctataattttcttctttctgcttgctttgagttctaatttttttaggttcttgaagtggggacctatattttttacttactatgttcttttttcttcttcacgttccaagattctcttatcagattcttactgtttggagaacttcctttatccattattgtagggtacatcttctgtcaataaatttcctcttttcttttgtttgaaaatgtcataattcccatttcattcctaaaggttttgctgggcataagattcttggttaatagttctcttctttcaacttttgacaaatgtggtgccacttctttctaatactaatactgtgtttctaattgtttctaccctatagggtcagtttttgtttctctttagctcttttcaaaaatttttctttgtcttcaagttcagaagtttaactatgatgtttcttagcatggaattctttggctttaacctgtttatagttggctcagtttcttgaatctgtaggtttatttcttgctaaatccaggaagctagcagccattatttctttgtgtacttcttcatctccattctttttcccctctcctcctgggactctgatcacataaatattaagtcttttgttataatctgacaggtcccttgcagtctatttgagtttttatttttttagtctactttgtctctctcgttgagattatctttaattgttctatctccaagttcactgattcttcctttgtcctttccattttgatgttgagcccatccacacaaggttttattttgattattgtatttttcagtactaaaaatcccaattgattcttttttataccttctatttctttgctgaaactttgtatttccttgctgaggtttgtttaaaaattttatttcaagaattactgtatttgcttgttgaagcatttttatcatggctgttttaatatctttttcagataatttaaaaatctttgtcctcttggtgttggcatttattgattgtcactttttattcagttagaaatctttctggttcttagtataacaatttattttctatcgaaatgtagacatttagttgtataagaagaagaaaaggagaaagtacaactactccatcttctcagaagtagaagtccctaattctaaacatattttaaagcagaatttttatatatttttattttaggaagaatacttattacaagaagtggcttatcagtaataattatatatatgtgtgtgtatgtatgtatattttatatataaaatgtgtatatatgtataaagaagtgacactatatatgtatatataatttttcagattcataaatatgtatgtaaacatttttttaaattatttatttattgatgagagacacagagagaggcagagacacaggcagagggagaagtaggctccctgtggggagcctgatgtgggacctgatcccaggaccctggaatcacgccctgagccaatggcagatgctcaaccactgagccactcaggttctatgaaaataattttttaaagtcctttgatcaatcccacatttcctcacatgaaataatatttcatatgtacaaagttattattttgtaatatctttctatccacttcatatcttagtttttttctgaattctttttgtaatattcttaataggtatagatctatgcaaaaatgcacatgtatatatgtttagattaaatatatatgtaatttttctgataaccaaatactagtgtcaacataattttgtcatctcactctacgcttattttttcatcaagttcctccttaatatctaaaatcttaggatgggggtgcctggatggctcagtggttgagcatctgcctttggctcaggtcaggttcccggggtcctgggttgagtcccgcatctggctccccacagggagcctgcttctccctctgcctatgtttcttcctctctctctatgtctttcatgaataaataagtaaaatcttctcaaaaagcatataaaataaaataaaataataaatacaatattaggatggttgtgaatattgctgaatattcatgttaaactattctattttgatctctttatttgaacagtaattgtatcaatctcattaataaattttaggttgaacaatttattttaaggagaaaacttctggcattttcaaattttaatgccagtgtttagaaaggatacctatttgtgtccttgaaatttaaaaataagcataactgggatccctgggtggcgcagcggtttggcgcctgcctttggcccagggcccgatcctggagacccgggatcgagtcccacatcgggctcccggtgcatggagcctgcttctccctctgcctgtgtctctgcctctctctctctctgtatgactatcataaataaataaaaaaaatatataaaaaaataagcataactacttgtatcatatttttctagttaggatatcttccaaggagaaatcctttggagattattttttaatagccatttctgtgattttttttacatgtttgttttgtgtcctatctgatttttgtaggcataatactagtaatataattatgaaattgaaaataaaagtgactcaactagttatggtaggctagaatattaaaaggtgtctatattctttcttaaataattgtctacatggtaaaggtaattttcttcttctgattttaaaaacagtgctactgcatttttaaaattcagtgctatataagctgtagcatttctcaaattaaagtgtcatagtgtctcaggggaagaaatggtagttctcagattcctactgggaactcttactcttttgaattctcatctgtgtctcgaaatatcagtactggaaggaattatagaggtgatcttatgcctaacttatacatgagtgtggtaactcaatgacacaccaatctgtctattaaatcttatatactttggggaagaaatgccaagtttatctcctcatacagaaatatcactaactagataatagcctatgcacagctctcatggccgatggcataagttttaaggaaaatttataaactcatgttatgattttaatattttcagggcctgtttcatcagtttgcatagctaagacatattcttttttttaaatattttattgatttatcaatgagagacacagagagaaagagagagagagagagaggcagagggagaagcaggctccatgcagggagcccgacgctgaactcaatcctgggtctccaggatcacgccccgggcgaaaggcaggcaccaaaccactgagccagccagggatccccaagacatattctttttgaagcttaatgtacataaaataaaatatattgccagtgatgttttgcgccaccactcttatcaaaggcttcctgaaaagaccaagtaattagtaatatgaaattgtcacatctatatttatagaatagattccactcaatatcaatacccaactacataaataatcataatcattcttaaacttatataagcttaatttaatccacataataaacatttccatgttaattattataggccttagacaattccttgttgcttacaggatgaattagaaactttatattatatatacaaaactctttttgaaatgcccacttccttctttgtcattgttctgcagttttattgaaattccaaaatccttaaccagatcatgctacgatacctccatgactatatgcaagttgtttctttaatccagaatactttttatatatgtagcaatctcactttagctcatcccaaaaaacttttctgaccatttcatcctgtgtggaattgagctgcttccctttggcctgcactgtacacccataaagattttctatcatagtacctagaacttaattcattatccatatgtaattagatgaccatgaattccttgaagatgagtttattgttgaatctctcctcctttttaaggacaaaatgctccaaagctaatggcttcacatagtccgtagcattaagtagattaatgttgattttgctgagcattcttagccacaagttgagaagactttcatcttctctccttacttttaaggagaacttttaaaaatctagagaagcctgaactgctttattatttaaagcatgtggatatgttgacaaataaatagagtaacaaaaatgtcttttgtttcaatccagtcatttaatttttgtgattatgtattgataataagtctaattttatttggaaatcatttcaaaaatcttaattaggaacttttgttcacaatcctctctctataccaatctggcaatctttattgattggtccacctccttagatatagttatctatgtctcaagttaaattggtctcagacatagcttcacaaaacacaattttaaaagaatggtagctctgtaaccagttcgtcatccatagtagaattctacatggttcatccccaaatgctcagtttaggaaaatagagtagatatatggttattcaatagctggggcaaggggaaaccaagatgaatattctccctcactttggcacagaaatgaaataatctatttaaaactattccatttctgatgtttaaattgaagaactggacttatcattttattatttcttttattaaaatgtgacatgtaggggcacctgggtgactcagcggttaagcctctgccttcagttcaggtcgtgatcctggggtcctgagatcgagtcccacatcaggctctctatagggagcctgcttctccctctgcctatgtctctgcctctctctgtgtgtctctcatgaataaatgaataaaatctttaaaaaataaaataaataaaaataaataaaatataacatgtattttaacaaaatttagtttcaaaacaaattccagtgttatcattgatctgatgtaatacttttttaaattagtaaatatttaaataaagttactcatccagacaaaaagattttttatctattgcatctgtttttccgttgttttaataactgacttttagggttacttgggtggctcggttggttaagtgtctgcctttagctcaggtcatgattccagcgtcctgggatcaagtccagcatcaggcttcctgctcagtggggggaatcagcttctctctccctctgctactcccgagcttgtgctctctctctcttgctcgctcactcactctctcaaatgaataaataaaatctttaaaaaaaataactgacttttaggtaagcctctaatattttgtactttcctttcaaaagaggtgtcagtattattagaaagattaaccagtatttactccagctaatttttattatgttgcactaagatgagtttctatgtaaataaatgaactaagaaaatttccttttcgtttgaaaacagaaggacatggatgaacttgatgatattctcacatccatattcaaacatgagataccatattatgagtttcgatcattccaacctgaaatcgacccacagaaagaatgtaagtattttccagcttcatcaattattcactctgtgcaactatttaataaatattatagctaggaatatagctatctgcattggtgattaaatgttttagacaattcccatcccctggcaagaaagcacatttgtaacaaacttctaaagctccatttggtaaattgttttatcacaagccctaaaaatgtgcctaacctttgcaatcagcaaacctgcttcctgattcttattctaaggaaatttctagataattgataatggagattgttcatcatagcattatttgtaattactgaaagtagaaataacttattcttccaaatgtaggtaattgattaaataaattttggcacatccattaatacaacaatcataaacaaattttattatgaactgatttgctggctggagtgatgctttgatatatacttaattgaataaaagcagggtacatagcagtagatagaatgtgatctgcttaaaacagtacatagctttagtagtatatatgtttatatatgtgataggtagaaagagagtctggacaaacatgcacctgttaacaatagttgttcaatttaaggtattacatctttttttttttcattttacttaatctgtgttttctacaatgactcttatttgtatcataaaaagaaatatgtatacgtatcaagaaaagtatatagaggagctactacaagagaaatcatacttctaaattttatcatgagctcagcagtacgttttcaaaattacctgttcctatgtagtttgttgacacactgtttataattcacttatatgccgactatgtgccatacactatgatagagacataatggtgagtaagacaggcatgaggctaaagaataggagaggtctagtataggtatactgtaggactatttagcagaggctttttctgtagagtttctatgctatttgaagtagttttatcctcaggttggcaagcctcagggaaacaggaagggaaagagtaggagcctgaaatcaccggcaattttgaattacctagatgtgcaagaacccctaagatgtttacattatgagtttacattatgtggaaaggttaataatgggctagttttgaatgagaaatcttatgagaatggtgaaaacttattttattccccaaatgcattttaacaatatataactcacaaaagttagactttaagttaaggtagaaaaggaacataacatgatatctcacttgagacactcataatttgattgggaaacaagataatgtataagtcataatagaaagaaacaatgtactaaactctatggaattgattgcctcaacaataggattttagagaaaagctgtattatttttcaagacttgtttcataaaggaggtatgatctttacgtgagtcttgaataacgtgttccaattaggaaagagaaaagaaataggaattctatgcaggaaaaagtgctggaaagatatgagatagaattattaaacaatttttcaaattccttcagcatttgaatttatttaggttgtggaatgagaattatggaactgagagaatggaatggcctctcatttttacgggttaagaattctagctaagtttgaaaagtgcttgttaccttgtttaggctgtaaaatgtgaattttagagttagaaatgatggaagagctctggtcccttatttttatagaaaaagaattctggatacgttaaatcacagttcctgataatgtggatacagttaaacttctgcttttctagcaaacttccaggtaaagctgatgctgctagtctcagatctttcatgagtactaagtgtttagagcagtggttctcaaacttagtgagtattatgattagctgtggagcttttagaacatttgatgcctaaaccacatccctcttccacccccaagtaatgaagaatctgggaatggttccagctctcagtagtttttaaagttttctaggtgattccaatgggcagccaagtttatccaccccttgtgtcttcaacttattacagatttctacagggaagtaattcatcttcatactgatctttggcataaagaaaatactactctctctttacaaaggctataaaattagtagagtctgaaagttttgaatcattactagaatttaataatcattatatatttgaaagagatttttaaacatttttgaaagaaagctacaaatgaaaagttcaatgtaatctaggtatttcattcagaaacctcattgctggattttgttagatgacggacgctattaaacctgtaacatgcaaacaaccgtcatgatatgaggcttttactcctagtggaagtacaaaataacaaaatacaaatcacagaacaaaaatacccaagatagacatgaatatgactgcagtcatgttttgcttgctttcttgacctcataatcttgaattaaagtcagcttcagaatacctggtctttaagaacttgcacctggattttcatagtgttcaagttaaattgatgaaaagatttgagaggcctttatacacaaagtggaattaattcaacaagagagtttgggaaagaaatggtaggttgttaagtaagttctgtagaaacatttctagatatgaactgagcagtaagttcagcaaacagtgaatcaggtataatttcagatatctgtccctgaagacagttctacaattatttggtgaaatattttgcttattaaatatttattatttattactctatattttagatgaaataaattggatgttaggacactgtgtgaagaagatagccataaggaatcggtaggatgtacttgaggggtgagccatggaattacatacagatagctatggtttttaaaaatatgatgctaaatgctatattaatcaaaactgttatatcattgtttcatcctcttttcttctcaagttgactttggtaaattctgggtttagttccctgtcacttcatttataataagtaaaatgttattccttgccattatgccttggtcattttcactcctatctaccaattaatttccttgacttttcttttccccaggcaagcagagttggggtatattatatctctataattagagataactcaaaaactatatagaaatcaaatctactttagtgttctcttaccctataatgctttattattgatttaaaataatattttccattgaggagaattttccataagaaaaaaattctccctaggagtagaataataaaagggttctaaatgatcttgctatgttagtgtaacatagaacttttgaacacaaaagtcatcaaaataattcatgaacaactttccagtatgtgaatcatcaggggctacgaaatcaatatatggtattttaacaggcaattttctactgagacatgagatactagaataggaaatatcagagtacattccttaaagcaagagtatgatttttgagaaaattctgttttggttgttgattatataacacacgtggtatatataaacacacgtggtatgggccactgtgaaattgattcgaatgcacctgacattgtgtggtgatgagacagagcagcacagcggccagacaagactgctttaacctgtctgcagctctcccctgtttcaagctctctgcagagttttgggaaagtcttgctgtggtagttacatgaatttccctaacttcagaacccttccaaggactgggtcataaagagactcccctgtcatggacaggaaaaaataaatagcttggcttgtttatttatttatttatttaatttatttatttatttatttatttaaaatgctacagctgctggttcaatcaccaatgggagttagttcatgaaaaaggaaactaggttaatttaaggcagattatttacctacacccagagaagagtactggtttaattaagcccatgccaattttttagaacatgtcaaaacatatccatttacaacaaatagcaatatcttaatctaacataagacagcatacacttgccagtgacaactgaaagtattaactgggaatattacctccatatatgtctataaaatgtagagcatataactgaatattaggtacaaatgtagtagtcactgcagctctaagatttgtcatgaacttgtaataagtttaggttcatagaaaatagctcatttaccaaggagaggagaaccacatgctattgtgctctttttgtatcatcaaataatttaaacatacaggaaaatatagagaacaatgcaaaaaacacccttgtatccatcacctcgcttttccaaatcttaacattttgtcacatttgcttcagagcttctatttttaagaaataaaatgctaaatatctagttgaatttttctaaatctccctctgtgattccacacacttgaaaagtgactgattaaatttcttgttttaaataaatttcttgatcattctcatgcagttcttcattttttctagatacatttgtttccataatttatgttgtacagttttgtatatttcatgtaaatgatatcacatttttcccttcatcttactatttttgcccaatataggtttttttttttttttgtatttatacacctgtctggatacattgctattacaatacgttgactatattccttatgccaggccttattcattccataactggaaacctgtattcctttttcattttaaatcatttgagaagaaaatcagaagcaatgaaaaaatcaatctgaagggtaaatggtcaagattattcctcattctgggataagaaggaataggtggtagggaaaaaaaaggtcatgtaaacacactttaaattagttacaaacacatcactgtttagtaaatacactaaattagggatagatttacacataacactaacgtagtaattgagtaatttggagatttagtatgtaaattgactgtgaaattacagatttttttttcacttcattttcttgataactcaacctataggtatagattttcacaacttctaccaaggacatttttagcatattctcaagttacttttgtgagttgatggttcaatttttatcaaataataataaaataaaacaaccactttgaattctttaaaatgttttccacttgaataaccaaaaaaagagttattcctttagtattataccactgttactcacttttacagtatttctttgaggattctttatttctccagacatgaagtttgaaaagttatgcttataagcatcataactgtgattgaagaatgtatattttttttaaaagtaagaaaagaatgaaggaagctaaaaagataatgttgataccaggtccaaaatttggcctttttcacacttaaattattctccagaaaaggaagcaccactggccttgagtccatagagagcatagataagaaagaacagtatgtcctacagaatcagagaaaccttcctggtcttaagatgtatcctaacactgtgaagataagcaatgtgctatctggagtcaagcaactttttatgatggtcagccattcacatttcaacaacatttgtaattctttctcattatgcccatttacattactgtaacaacacatattctataatatttgagaattttgtaattatagatgaaaaagatgagccttcaaatgtaatatattctcaagttaagagaattttgatctaaaagattatattaagcaatcttaggttttcattcatattataagtgtaaacaaagtttctcctcaaactctgtatcaatagatagtatctgtagagcaatctgtagagcaaataatgggaagaattatagactttatttattcatgacagacagacagagagaggcagagacataggcagagggagaaataggcttcccgcagggagcctgatgcaggactacatcccaggaccacaggatcatgacctgagccaaaggcagacgctcaactactgagccacccaggtgcccataattatagttttgagtaactccccaggacccccaaaattctgcaaaaaaatctaaaagtaactggaaatacagactaattcagagtgaaataaagcactacacagaagaaacagaaaagtatcaattaactgcctcataggttcattttccatggtagtgagactgaatttcatttttatatgcccttcccccattttaagcatctccaaagttgtaggatgtgttcacacatacacacattagtatatgaatagaagcctataggacaaaattgtgactagagcaatgtataatacaagggacaaaatagcagataaaaaaaaaaaaaacaaaaaaaaaagtaacagaccaggctgagaatccaaagcatgtggtaacagcagggttaacagcacatgataaagaacaatagaggcttaaaagctgaagtagtaggctgcattgtgtctagctgctttcgtagcatttctatcctgagaaccagtttctgacttggctcattctggaagccagaaaggtgagctggaaaggcaaaagttgtatccttatggaagaagttgttgataataatggaaatatgggaaataaatgtgacctctacccatgatacctgactgactataagaagttatttcctccatgctcattatccacagaatctgaatagacattacctctataagaatatgacatgtgatgcatggttgaaatattatttctaacaatgattatttctgagattgctgtaagactgtacattcctccaggaaataaaagtatatgtattttagcattaagttttgtcttctattgtagcctttaccattgacaacctgaaaagatattttaaaatatgtaagtcaggggtgcctggatggctctgttggacatcctactcttggtttcggctcaggccatggtctcagggttgtgggattgagcactgagtcaggttccactggtccctacagtgtctgcttggccctcttccttcctctctgccccttcccctactctctctttctctttctctaaaataaataaaatatttttttaaaaaaatgaataaaatatgtaaactaagacagaagagggaatctcttggagaaacagtgaatagaatggagaagaagtactaccccagacatactttgtatattttacagttttattagcaatctgtgaggttaacctcagttttcctggatgtccactgttgcttaattatttgaaccaagccccattctttcttgtatttagcattttgctcataaactttagtggacatcagaattccttggaaggtttgctaaaacccaaatttctgggccctactcagaatttctgatacagtaggtctgggatgggcccaggaatttcaaatttctcacaaattcgtagatgatgctgatgctactggtctatgagtcaccaatgaaaaccgctgagtaaacacagcaataattggcagtactgtcatatacataaataaaattgacatgaatccaattaaagtcaagatagcagatttatttttatcttatcattccatgtcccagaatttaaagtctggctgcaaatgaagtaatcttagagtgcttatctaaaaaatatattttgtgtgtatcattagaaaaaacaaaaaaaccctcatgaatttttagacgttaatgaactctgctttataataggcactcttccagatactgagggaaggtagaggaatatgaaatcagtaaaatacaaaccatacattcagagggcaccaatgacaagggaagagaagacgtcacaaaaataagtgccactcatagagtgtatgctctactaataagagctaacatccacattttgcatactctatgtgaagaacaattctaagcactttatttaccttaactcatttaattcctcacaagaatccctaggaaagacagtacctgtcttgattttctacttctgatctgtagtatctgacagcagaatggaagaaatgacatatgagctttctgaatatgctgcttgaattctctactcaaataaccaagacacatatgttattcttctctgggtccacacaaaaaaataaagggttttcagagacaaaagtaataagtttaaatatgtaagtttatcatagagttagagaaaatacataaacattgtccattagtaactggtaacaggaaagaggctctgtaatagccatttaataagaaaaatacatgcatgggttagcctcaattatatacacacactcacaatggaagtggaccttttattataaattgaataagttattacttaaggaatttcttcttacatttgcaggtgatgtgcagtagtgatttgtagtgtcactgcatgtgtaaaaactcattctattgctctgagaaggatagtaactcaaagacacaaaatagtttttatgtcttgtagccataagttatggtttaccaaacccaccatggtcattttcttcacctcattaggcaggatgctccagagaaatagaatagatgtattaagagagacaaagagacagagggaggtttactttaaggaactggctcatgtgattgtggcgctggcaattctgaaatgtgtacgtcattccagggggctggaaatttcagcaggagttggttttgttgccctgtggtgacctaggagctaagtttataaccactatgctctactcaaaggcccatatgtctaatttaacttattgctatgtaatttattgtgacagctaccccatcaacacccacacctatacttttccatgaccaccattgtcactttccctaataaatttctctttcgcttaccttgtaactttggagccatgaacctgaatgttatcttaaattcacctctttcttcagcagactcaatcaagtcctactaacttttcttttaacgtgtctgatatttctgtccttccttgtccattccaggctgtctccactctaattcaggccatcagttacttcttgttcctgaaaaaaatcttctagcaggtatttctaacatgtttcattttccatcctacacttcagtgctccccaaacattcttatgttccatttttattctgtccctatgttatgtatgatggtgcttccaaaggcatcttaagctctaaccagtccatttattgtttccaatactgcccagattttcctccttgccttagttcatttttattatcaaaattatttaactttttttttaaagaataaatccattgctaaagactataagaagtcctcaaaaatgctgaatactcttgactactgaaatcaagcattcttgcctgatatgttgatttactgcttcgtggtgcatatcttttggatttccatgtgcaacccatacaaaggccagtggacagctttctatctgtaagtcatagcctcatgggagagtgtacaactttatccagagggcttccacacttagattgttctaggtttcctgattctttctagacaccattctgcttcttcctcgcttcttagtgttttatattgtcaagattcatcagagacaaagttagtgattttcaaaagtcatttttaaatgaacacagagaaacagaatacagtcacacatctagagaaatgaagtttctagagacttcttattttatttatttattttagagattttttattttaaacagagattcgccacagattgattggattggttgaacttggagcagccttacttggggcagcattactctttttattacttcatcactgagtttgatactggaaaatggataataaacctgttcaaatgttgtaagatttaattttaattgatatcattccaagaactgttaaatgaggactctgtatgctcctttgcttcttaacatcacctttaggcatggggtcccagctcccaggctatttattgtggcctcatggattgcttcagtcttcttttgcatgctgccttatttcaccctggccattcctggcccactgccctggatgagaagatgttttacagtgtgtcctcaaatactctttctctgggatcagaccatcttgcgtcaacctgctgcttaatgttcttctgctctgctatcactcattccaacactatgtgtctgtgattcctccttttcaactcctttcattcattagttgtcatatttccttcccttttcctttctcttccttcttcctcttttcctctttctcctcttattactactgctgctaatttttggtggattctatttgggtacttaggaacaatgaactttttctggattcaaggtcacatgctccattcatttcccatgcttcatatcagttatcattcatatgttttgcatgccagaacatcaatcttccagaactttattttttttagttttataaaactcccaaattttaagcatttagttacaacaaaatatacagaatctaccaaaattaataagatgtacaatcactcaaaggaatttgtattatctgagaacggcacggtaaggattccctttcatactagatttttcctattgttttgtctaagcatggtaagtgggaaaataggcaaaacagagtcatgccctaggactgtccttcctacttccagagtggttgccagtgtggactctaaacatgcccagtttggattttaactttgtaggctctaaacacagagacaagcagggaaaatgcctcagaaatcataaggtgagatattcacaggttcctcttctagccctatgcctttcctggcctcactctgaagcaacacaaaggactgcactcagagactccttcactgtgtggaagaaagaaaagaaggcaccatattttgaaaatttctagccgtgttgcaatgggaaacaaattctgaactctctctccaaaaggagaaggggctttagattgcattaaaatgcaattgattgtcattgttcacagtagttacatattatacagtcgctgaatagagaatactgaacattgctttaggaaaaatataggattagtttcttgcaaacctctggtcacatttttgtcaacccatcaatacataaactcatttttttgcatgtttctgtttaaaaacacagcatttatatgtatttttattcattaacattgaactcattaacaatatcatttgattcatacctgaacaaagtttatctaacatacttattttcttcataaagcatatcacagccttcttgtgcttgggaacaccaaacaacacctctgtattgtgccagggagccactttgtaaactactaaatcaccaccaacatccaaaaaaaaccccacaaaaatgggaaacgtagcactacatagaccacaaggaagacatttgtttaaagtatgagagttgaaattagaaggcagggtgttgccttgtttggactcagctgggaacatgcactttgggagactcagatttttcactgctcttcacatgtatacaaagaccacaaaagggccacaaattttggtttaggggtggtgggacacaaataaatttttgtgagttggaaaatttgcaaatatggaattcctgaatagtgaggctcaatagtagttgtttgtatgaaagaatacattggacctaggtttaatagcttattgattaaatgttattatcttcatttaaatgttgagaaaatcgtgataccaagttcaagtagcttatccaaataacatcattagtaaatgctagaattgggattctatccatttaatctggtttcaaagccctaatgcttaagcactaactgcatgatattgactcccaaattaagtaaatattaagttttagaattttaagatttataatactgaaagatgttattttgaattttatgtattaagatgaaaccaggattcatttgaatttaatgctgattaggaatttaactgttttaactaaaaaaaaaaaaaatctgttaaaaatctttaaaaaaaaatctgacatgaagtttcctgaaacaattaacaagggacatacaaaatggatacaactttcaatattttctcataaattttaagaagctattttatttttttaagattttatttatttattcgagacagagagtgagagaggcagagacataggcagagggagaaggaggctccatcccaggatcctgggatcatgacttgagccaaaggcatattcccaaccactgaaccaccccaagaaaatattttagtaacaagaatataattgcaatagttagtgaggtaaatacaaaattgagtttaatactaaagtgtacttctgtgtacttcccactcgtattttatgtaatacaaaagataacaacaaaactgcagcaagggtattgatggaccattatttccctcgtgcatttaatttacattgtcaaggcagttattttttgtgcttgaatttctaactaggattttatgtttactatttcgacagggttgaagaatggtacctcacatgtagcatttgtggttctcctttcatagaacggattgcacccagcaatgctgttttgcagatccacagaaatatgaatgtttttatcagaatccaaatcctatgatgaaagaacaaaaattattagctttcttcaaccttcataaatattatccttactcttttaattgatatctcacctatgacatgacttcttacccagaggaaaatcagaagtccccaaccctctaattttatacatgaagaatcagaggttcagggagacaaaggaattttcttaagcattacagatttagtggctgagcttagattagaaagcagatatcctgactcgtctcctctttgttgtgtgcttcattattttgtcctaaagtggccacctggtcattctactttgagtgaataaatatttatgattctagatagtgatttatgagaacaaatggattacatgatcatgcacttccagtttccatagtcatcttttaaatttttaaagcttatattctaactttgggtatattccccattctattattctggtgagcaggattaatcttttgaaagctttggtttgattacttcaatctaattattggccaccttttatagggattaccttgaactgttagaaatggctcagcacaaatatatgactcaatttctttactatgtaccaatttattcatttttatttgcaaaaactagtgaaattgaaatttccttgaaaatgagagacatttaattctgttctttgaaattaccattcaacatactttggttgaaacaactgaaacttgaaaatgtatttccattaaaaaagtcaaaagcaaactgactatactttaatgtgaattgttgctgcttatttacaccaagaatataaaaacagatgcaatgaatcttgattaattcaatgaaaacttccatgaaattaagcagatgtacgctatagattaagcaaactttacatgttttttatatcttcctaagaaatccattattttaatctaataagtaagatgataagctgcttataaaataaatttaagtttaagaaaattgctaaatggggatgcctgggtggctcagaggttgagcatatgccttcggctcagagcgtgatcccagccaggatccaggatcaagtcccacatcgggctccttgcagggagcctacttctccctctgcctgtctctctgcctctctttctctgtgtctctcatgaataaataaataaaatctttaaaaaaaagaaaattgctaaatggtgtatgagctaagactaataggatttctgtttctccttaagatgtacatcacaagagtctactgctcccattgtaacaagaagcaagatgattttccaaatcagtctctttttttaatccagctgaaagtcaatgacaaaagaaatctaaataaaccaaattccaaaaagggaccagtccttcacaagaaaaaagaaattcaagacttccttcttcccttacagagcaatccactattgatgagaagtaaagagaaaccagctaaacatttagcaaactttaaatggctttattaggctagtgtgatggacaaaaatctcaaaaagcccaattacagatcacccaacacctctcttgtaggctgttataagtgcatagagctattaaaccaagggctggagggcaggaaagctgagagagatccttgaaaatattctgagtcttccctgaaggcccactagtgactgctgaaaagtgaaagggggacaggtttgctgagacaaatcccaccaaagcttttacctatggaaagcaagttctaaatacagagaggcataaaagctggaagaaatacctccaagtgattgggatgggatggtaaggagtctgagtgtactatagaggccccacaaaatctgagagagatattttgatgtgaagaagtctggaggcaggactggaggaaaaaaaaaaagaattatctgctaacccaaaggtgacacccaagccactgaggtttcttgagtcttaatagggctcagatcccaggttgtgtggaagaaagctgtgatcctgccctcaaaatatctgaagccagttgttaattgaagctaagtaaagatgcaaaaatgaccaagctcagttcacctacagataaatttgactctgatcccaagtgttgttgtctgactaaagaaatgctgtgccttattctgggggtaaatataatttacttcagttctttaatacaatgctaagtgtataacaaaaaaattacaggaactatgaagaaggagtaaaaggtgattcttggtcaagaaaatagacagtgattagaagcagattcagaaatgttccagggatccctgggtggcgcagtggtttggtgcctgcctttggcccagggtgcgatcctggagacccacgatcgaatcccatgttgggctcccggtgcatggagcctgcttctccctctgcctatgtctctgcctctctctctctctctgtgtgactatcataaataaataaaaatttaaaaaaaatctttagaaatgttccagatgttgaaatttttagatggaccattaaaataattatgataaaatgtgataaattctattgtaaaagttgggcaacaataattaattttaaattaaaattatttttatcagaatagaaattatcataaaaaaggaaatcctagaaatatacaatatcagaaaaattttttaaaaattagatgggcttacttatagacaaaacaaagaagaaaagagaatcagtaaacttgaagacagtccaaataaagtattcaaaacgaaaaagagatgaaaaatctagactcaaaataaagccataaaaaagaacaggctcagttcatctacatatcaatttgattcaattctgatccaagctgtataggataatattaaataatctaacatatgtgtaattgtattcccttagtaaaggtgacagaatgaggcagaaaaatatttgaagaaatattggccaagaatgatccaaaattgatgattgacatcaatccacagattcagtaagccctgtaactcccaagcagactaaatacaaaaatatatacctacacacatcatagttaaactgctgaaaattaaaagtagaagtaaaatcttaaaagtagctgtggggggagaaggagaaggtgaggagtttgtgttggagaaagcgcattccatacaggaaaacaattgtaaaaatgatatctgacttctcatcagcaatagaggccagaagacaatgcaatacataagtatatatggttccacttatatgaaatccaagaataagtaaaacttacctatgttgataaaaattagaaagtagtccacttctaaagtggagaattgactagaatgagacatgagagaatttgctgtggttatcacagctttctttagcttatttttttcggttgtggttacttgagtgcaaagaattgttaaaactcatcaaactcatcagtgcaatttgaaactatacttttataaaaaaatttaagccctagaaacataaatatctttaaaaaagaactatagtgatcgtgtgattgcaaacatacctgttccagatacagaggaatccattcatatctcaaattctcataattaccaaatcagggtgaaaatataaaaagtatgattttataaatagtatatacataaaatcctgtactagttcctaacataatcatacctgtattgacataaattcttttattcatatataaaatatattatatccacatactgagtatatttaatttgatgttctacaaaaataatgctaaagatcatgatctaattaacattaaaataactaatatagctcctatgaatcatttcaaattctttaaaaagttaatgctttcatattttaattaaaaaaactcaaattagaaatattttagttattgaccatgtatattagcaggctgagcattccctacttaatgaaaataagcagacaaaataacctcactcaagataataaaagcattgctgtaaatgatcatttaggccagtttaaaaccaattcaccattttataaaagagcagctattctaggggcagattatcaggatggtcagtactttggaataatgaaaagaatcagggaacttaaggttggaacaaagatgaattaggacaaggatgctttatttgacagagaagagagaaagggaagaaaactaatattttttgagcgtctgttatgtttcagacacttctctgggcactttttctatgtccagtcacttaattatcttgatagccctataaatttggaagagaattaagtaattagtagcttgttgaagttcacatactgaatactgactctaagcctgtgctcttgtgactattatattctatattgcccgaaggatatgaatacatgagaattgagaagaattacctttcacctcaacagaagtaataattgtctaaaatcagtgttgtctaatcaagacacagtctgccttaagaagtaaaggacactgtctctagaggtaggcaagcagtacccagagcatctcgtataaattctaggaatacaattctaatactgggtaggaaactggaaagataccctttaaggctctttcatgactataacttcacaaaatatattagtatattagtggattttaagctaccactaaaaatgaagtaaataaatggaaaataaagaagccaacctttaaacctgtagcaccctttagtatcatcactataaacttaaatcagcagaacttccatttttgtatcagaaaacacagtttaaagtagcacaactattaaaaaatgtaccctgaaaagtcatgaacacttgtcacggtcctgattactctctttgatatacaagagagattcttaatgaaggtggttgttgattctaatattatagtaccttaagcttgagggatttactgacaggaagtcagtgaggttaaaaagtgactgcattctcataatgattaaacatttatttaacgataaaaagattttaagctgcattgacggtgatttaaaagttcaccagaatagcaatgtgggggaataatgcataatgcattgattgccaggtgatggtttttttgcttattgtatgatgtgtcaatgagagagatgaccaaaggcatgtaaaaatctgacattatcatgtaaatattttttcattaactttgtcctggtttaatgatcgaaaattttgtttatggtaccttttaatgaaaataaataaaaattatatcttaatacctttttaagaaactagatttttatttcaaaaatggtactactttagacagtttcagctttcacaagctcatcttcataaaatatttggtaaattttgacacactgcataccatataagaagggaccggtgtatttattgttgaaatataaccatggaatttacctatggccatttagtattaacacattttgaagggtaaaatatttccttacagtaagttcttaatgtatcctagaggtttatgttttgtttcctattaaaccataaaaaccaataagctaaataatttattctgccctgaaacacccaagtgaatataaactcaaaatctaactccccttatttcccttttttcccctttaatctacaaaatagcaaagaggtaaataaagccagtttcagttttctaaaacattatggtagaaaattgacttttgctttgacaccttcgtttctgttgctgtaagtctttgcattacttgcttctgataatgccacagctattcaagcaagctggtgtgtcatttctataacacattttttccagctaggtgtgaaaatatagaataataacaaaccatcactattctgtgtgtgttttgtcattgcaatagctgcttgcattcaccattttataattatacataattaatacttctaattgatagctaacaactaattatgaaattcaaactccaaaacattgatacaaaagctgtaaaatcctgatggaaatttccatactaaaaatatttaaaaatatttataaaagtactcacctgtattttctgttgcagttccttttgaaacaaaaatttcacataaagatctaatataacccctccagcaattatgtgtccatttcaccttcagaaggaagcaaaccaggcatggcaatagaaaacacagaaaggttttatagatggacctattttatattgttttttagtgttttgctaatgttgaaatatattcaagagttactgcaatatgatatttttgttctattttgttttatatttaagaggttgatgtgcctttccttttttttttaatttattttttattggtgttcaatttactaacatacagaataacccccagtgcccgtcacccattgactcctaaacttaccccacaggtaagactgaggcattaatttgttaatgagaatattagtttatatttattgtatgatgagaaataggaaagagatgcatcattgttaaaatgactgatggtagggggttcctgggtggctcagtcagttaagcgtctgcctttggctcaggtcatgatcccagggtccttggatggagccctacatcatcgggctccctgttcagtggggagtctgcttgcccttctggccctcacccaactcctgctctctctctctctctctctctctctcaaataaatatctttttaaaaagccactgatagtttcttctatgcaaaatgacactgaaagcctgttctactttgtaagaattacttttagtactagattgtgttcaactttttcttatggaattccagacatgataaaagcatattaatcctattacctcttatgcttacagatactcatttcaaagaacttccaactcttctccactgtgcagcaaaatttggtttaaagaacttggctattcatttgcttcaatgttcaggagcagcatgggcatctgagatgaaaaatttcgagggttcagatccagcacatattgctgaaaggcatggtcacaaagaactcaagaaaatctttgaagacttttcagtaaggtttttttttttttttccccattttatctcgagaactctttttataaagcttgtgtatatgtgtgcgtgtgctcttttggaaatgatgctattgctcagcaagtttgtagtgtcttttggtcctgagtcattcactgtgttaaaagcattttgcaagataggtcaacttgttttagtatcactataaaccagctctgactccacacttcctcccctaaatagtctcttttaaaacttgtctttctggcttctatatagcctgttccatattggagaggaaatggtgaaaaggatatgaaaatctattacctttgttctttcattcaagagcttaagtaagatactattacgctcatgaaatcagataataattattaggttaaaggaatacattttgataatcattcaaaaagtctattccataaaaagttaattcatgttaaatttgtttctcatatgtgattccttttctctctctgttaaatctcagattcttattatacaggttgaagtcctagatttcagaaggacattagtttagaattagtaagaatacaatcaaggtagattttgaagaaggggatactaccttagtatatagaatacactttgtcttttcgtaatcaaccttagaagagaaattatttcctcttgtaataggagaaacatatgcacattttgtttaagaaagtgaattatgcttctgatggctctttggaaaatataagacaataattgtcttagtgaatatgtcaaaatttgtctttagattctcagaagataaataattttattgctgatgaacagttaatatagaacattgaaatgccgtgggtcacttctggtttttttagcattcattctgattattaaattaagtcaatgtaatattggactttataaaccattgatatttgtgacttccagagagtgaaatgacaaccaatctaactttttccccacttttaagaaacagaaaaatagttctaagaatagtatagcgtggatttcattcacaatgaattacaataggatatctttggcttgtatttaaaataaatacaaaaatgaaatgaaatgaaatgaaagaaagaaagaaagaaagaaagaaagaaagaaagaaagaaagaaagaaaaagaaagagaaagaaagaaagaaatgcggttttgtaataaaattgaagaggttcctatttttattctatatccttcatatcaggaataagaattatttttatccctaaaaagatttcagatagtaattctcagatatctaatgagtacattcatttaagtaagtatccactctatgcatccactcaccagtgctacatggtatgcatctccattaagtcaatgaatcaatttagccttgagcatatttgcctacaatactcttgtgcagtacatcttggttgcctatctcaattttctacagccctattctcaaatttttaaccactggtggactgaagttaaaattttctgtgaaagtctttagcatccatggttgctttctaagataaaccctgattttcaatgagtgatacttccacttgtccttctccagtgaagacccagcttttaacaggtgcagtcccccatagataacatgacactagataataatgacagtagagatgaacataaattcaaaggtagcatttgatggatataggaaataattttctgtttctcttacatcctctgggtattttcacctctgctcctgcagtaatagcaatgatacaagttgatggtattatagtattattacctatgatagtactgaaaacatgaaagaaatgtcagtgttaattagataacattctcaaaaaaatgaaagctattcattcataatgaagagattctgtgttctaatcacattctgatcaatctaagattgtcatcactatgtgttcaaattttccattaaaaagactttccattaattattgtttcaatggacaccgagcctccttccacagtttggctattgtggccattgctgctataaacatcggggtgcaggtgtccctgcgtttcattgcatctgtatctttggggtaaatctccaacagtgtggaaggagcctcggtgtccatcgaaatatgagtggataaagaaggtgtggtttatgtatacaatggaatattactcagctattagaaatgacaaatacccatcatttgcttcaacatggatggaactggagggtattatgctgagtgaagtaagtcagtcggagaaggacaaacattatatgttctcattcatttggggaatataaataatagtgaaagggaatataagggaagggagaagaaatgtgtgggaaatatcagaaaaggagacagaatgtaaagactgctaactctgggaaacgaactaggggtggtagaaggggaggagggcggagggtgggagtgaatgggtgacgggcactgggggttattctgtatgttggtaaattgaacaccaataaaaaaaaattccatatattaatgaaaaaaaataattattgtttcagaattgttgaaaggagtagatatttttcaggtttgaggtttccaatccaatgaaccatacagaagaaatatatattttaaatgcaaattcattttgccctcaaatgattgactgatgtgttcaattcttcctaatatcccatcattgagacactgttaattttataatttcacagagaattacaacacagtgtgtttggtcagctctggataaagtgggtagaaggaaaaagcaattgtgatttttaataatacagagaaaaaaaacattggcaagcattcataagcaaataaatgccatccatatcttagtgaaaccatatgaataataaggaaaaagagaaaaaagagaagagcttcaaatcacacctcttacatttcttcatgacatttttcctttgagaagctaataatttatacagaatatacgagcagaatctttttattgtgaatgaatagctttcgtgttttgagaacattatctaattaatacttacatttattttcatatagatgataataccatatatttctaaaatccataaggtctttcaagtaaattactctatatttggtttctgacgatgtctctgagacattactctgaaaggtggatggacaaagaggataaggcttggtatttaagtccacaactgaatgtgaacctctcaacagagttttaaagaaatcccagactatttcaaataaaaaatagcaatattgacaattcagctttctgaaaacttaatcttttaaataaaaaaatacctgaaaaattaaaaatcaactggaacttggccacagattaagacatactttgtgattcaacatatacagcaaaaattagagtgcattttaagcaaaataatatatgatatttccttgtattatgtagaatattgtcactccatccatctctagtttaactcacttaacaaaatgatgacatcagtgtgtaaatggatagggagttatagatatgtccttctctattagctggcagtggagttggagaatagatgtttatttctcttaggaataacgttcagattattatcttttttcttacgccacagtgttcacttatgtagttttatgtatatgtgaataagatcttaaacataaatcattttcagtaaaattaaggaaatagaaaatcagctttccatgtactttctctagctggcttttcagaaggacttttccctggaaatatttctattgctcacagctgtgatgagattcctggggcatcttactctcattaacccatcccaacgctgctctctgactctgtagacagtggccttatgtgtgagtgtgtgtgtgggagagagaaagagagggagatggagagagtgagcgagcgagacagattcctctatggtctgtttgacaataaccctggactttcttttcaaatggtccagggtttagaatgaaggtgcagagaagtagatgctgaagcatcttgagcatcccttcataaagcaatgattgactgactgggataaagaacctattactcagaagttagactttatgtttaggccagagaacatcactcttcttgactccttatatctctctgtattagcattgctctctcggtgctttacatggtatttgctctctagtagattgttcatttaatttagcatatattagtacatactaattcagtctaaatagcagcataccctgctttgccggtgcagttttcctttctttttttttaaataataaatttattttaatggtgttcaatttgccaacatacagaataacacccagtgctcatcccgtcaagtgcccccctcagtgcccgtcacccattcacccccaccccgtgccctcctccccttccaccacccctagttcatttcccagagttaggagtctttatgttctgtctccctttctgatatttcctacccatttctctcccttcccttctattccctttcactattatttatattccccaaatgaatgagaacatataatgtttgtccttctccaattaacttatttcactcagcataataccctccagttccatccacattgaagcaatggtggatatttgtcatttataatggctgaggaatattccattgtatacctaaaccacatcttctttatccattcatctttcgatggacaccgaggctccttccacagtttggctattgtggacattgctgctataaacatcggggtacaggtgtcccggcgtttcattgcatctgcatctttggggtaaatacccagcagtgcaattgctgggtcgtagggcaggtctatttttaactctttgaggaacctccacacagttttccagagtggctgcaccagttcacattccccccaacagtgtaagagggttcccttttctctgcatcctctcctacatttgtggtttcctgccttgttaattttccccattctcactggtgtgaggtggtatctcattgtggtttaagaagcttttgcacaggaaaggatacagtcaacaaaactaaaagacaacctacagaatgggagaagatatttgcaaatgacgtatcagataaaggactagtttccaagatcgataaagaacttattaaactaaacaccaaagaaacaaacaatccaatcatgaaacgggcaaaagacatgaacagaaatctcacagaggaagacatagacatggccaacaagcacatgagaaaatgctccgcatcacttgccatcagggaaatacaaatcaaaaccacaatgagataccacctcacaccagtgagaatggggaaagttttactttcttataagcctagaagctgataatgggaaatgggttccctttccctcccactgttaggtgcacagggggtgctcagcatgtatttactcattgatgaggcagaaggtaagtgtcaaattgatctcacttacctgataatatccttaactaggtcccatgatgcaaataaaataattggttatattctctttatatttttttaaagttcagttttagagaattcctggacaaatatcccaactgctaacctcagcatatattttttataggccagcctacaatgaattttaaaattttggcttttgttcaatattgatgttctgttagaaccaaagaacctggaacaagacacataactgaacttgactgcatgcattacactttcatgtttcagttatgcttttggaactacctagcaacctctttctaaaatcccattagatgcaccagagtggcaattacagaattttacccttctgcaagtctgcaaacccagttttaatactctaattctgcagctcttagcattccttcaactgccatatcccctctcatttgccaaattttccgcttttctcttcacataggactttgagatccagccagctctggatttgaatcttgacttcatcaaataccaagtttgcattcttgaacaatctctttgctttctcttggctttagttcctttatctgcaaatggaaaaaataattccttacagagtgtttttaggacttgatgaaataaatgtgaaagtattaaatctatagtagatccacaatatctactttgttctttggctcaggctttggtattggtttccctgacactgccattctcctaaatctccactgaccccttatgagcttcttctcttaccccaaacagctaaggttcttcctatataatcttttcttatgattgatagctaagtcattgtctttaaccttggcctttatcttgacttaagactccagttacagtttgcaagcattgttatctttgatgtatttgattcagatgtgggaagtcttcgggatcctcacaagggtcacgcccctgctcatggtggctaaatggtctttaaagccccttgtgaccttaagtttttataatcatttgtcttctaatttctcagaaaccttccaaacttgagttcttttgtttttctctggtttcgttaaagccaattagcttgtgtagaacagaatagtcttgagctgtcacttacatgcctgctggtcaaaaggaaatgcaatttcagtgagtgcaaaattaatcacaaatgttaattagagcaatcagtattgacaccatagctttagaggctagacctcatgaaataaacacaatgacattatgctttcagaatgaaaagtatacaaaagcaaatgaattgaaatttcattgagaaaatacgagtcttggaaattcttgtctattaattggctttgaatttcagtcatttttgatgctttcatgtaatccaatgtagatgggatatatacttttctactggtattgtggcatggtttctactttgtataaagccctctccaaggtcagattatatatgagaaaaatgcagaaccacatatatttttaaaaaaggatattcaccaaatatcatagctctgagccaggcagtatgatttcttcattttccttatgtagcaacaccttccttgtgatatatatggatgtgagaaattaaagattatgtctgacttattttgcctccaaaaatataatataaaagaaaaatagatggtacctgttgaaggctaaatcttggatttgttttaaataaattaattaactttcacttggtccaggaaatcaatagtctctttaattttagtttggcaagtcaactcattgacttattcaacaaatatttatcaagtacccattaggtatcagtattgttctaaaaactaggaattcaaaacagaccaatttaaactctaccaggaaaaaaagacagactataaacaagtaaataattaggacatacaatgtgtcaagtgaacattagtgtttaagaccaagaagaaataggaaacactacattgagttgagtgatatgtggggagagttttgggcagaaattttgaaatgagtagggaagtcctcactgtaaaggcagcctttgggtaacaatttgaaagctatgaaaaccatgcagatatctggagggaaagtattccaataagagaaaacgagcacagcaaatacaaagacctggaggctggtatgcctggcatgtccataaaagaacaagttggccagtgttactgaagcagagtaaaagagatagaaagagaatagtaggggcacaagggtggctcagtggttgagcatctgcctttggctcaggccatgattccagggtcctgggatcaagtcacgtatcaggctccctgcggagagcctgcttctccctctgtctgtctctgtgtctctcatgaataaataaataaaatctttaaaaaataaagagaaaagtaacagtgtcagaaaggtaacaaaggccactatctgtaaagcacataggcaatggtgaggattttagagaataaaagccattaaagaattctgaagagagtatgatgagttctgatttaattctctggctgctgctttgccaacacacagtccaaggcaaaagcaggagggatgagaactgattggagaccatctcaatcatccagagtatagatgacaaaatgaaagcaggttagactaggataacagtaggagagactgtggaaagtggtcagatatgaaatattccagtattttacagaaaagctaaaagatttactgattattaggcagcagaatgtaaaaggagtagttactgatgtctccaaggattttggtacaaggaatgaattgcttgctatgtagtgagataaggaagattgtgtgaagaagcagtttgctggggaagatcagtagtttggatttggatatgttaaagtttaaatacctactagatattgcaaagtttagaagttaagttgatgagagtacgagcagaaaataatgagcagcagcccttgaagtagaaagagacatcagaaaatctattgacttcgaagctgaagggaggaaaaatttcaaagaggaagtagtcatctatgtgaaatgctactgacaagttaactaagataaggacagagaatcagcctttgggtttagcactgtggtcatcaagagcagtttgtgggaagtactctgagggcgggaggtgcagtcagagatgtgggagagaagtaataaagccttgacatgattagagagtcaagagaggaaaggaagagtgaaattggggaatcatgagtatataacagttttatggggtattttgctgtaaagggaggagagaaaacatattgtagctagtagggaacatggggtcaatagaggtgtttgtttttcatttttttaaagattgaagaaggggcacccaggtggctcagtcagttaaatgtctgccttcggcttaggtcatgatctcagggtcctgggactgagccccgcatcgagctctctgctcagcaaggagtctgctttcctctccttcggcctgccactccctccctgttcattcattctctctctctctctctctctctctctcataaataaataaataaataaataaataaataaataaataaataaaatcttgaaagaaaataaagattaaagaaattggggaacttgggtggctcagttagttaagtggccaactctggatttcagttcaggtcatgatctcagggtcctggaatggagccccatgtcagactccatgctcagtggggagtctgcttgaggattttctctctccctctccctttgcctctccctgacactctcagtctctctctaaagtaaataaataatttttaaaaaatgattgaagaaattacacactgtaaagttagtgggaatgaccaagaaagagagaaaaattagtggttctggaaaagtgggaaagcactgtcaaaacacttttgtatttcttaagtaggacaagaaggatgctgatatgaatgatatgtgtgtatgtacatttatgtgtgtatatttatatatttctccttaaaaattaatcttaagaaaaatgtctttccaagtgttgaaaaaatggaacagtgattcattgggagaagagaaagattaaggtaatattttctaatgatgcgtgagcgcctggatactttgggaatgagtttggtgactattttactctaatttagtttgtcccaaatatgtcttattagctgtgtatggaaacaagtatgaagcatattctatattagacatgcattccctgacaaagagttaggaatctctggtgaaaatcatctagaggaggaaacacacatgcagcctccagctgggatgagtgtcttgaagagaaagggagctaccaggcataaaaatcagagcgtttttcatctgtcagacattaaagtccactcacattgacaaatcttaaactcagatcctcaatttgagcctgtggctgagacttcagcctagacaagcacatttctgcctttattagtgtttaatttgcttttctctgtactgccgagcccacttattcaaatgtgactctcactaaaatctcagtaagccaattaatccatttccttaatgagaactctctattacaagaatgggttactgcatagatttatattgaatctatcaatcttgcatttggtggggagaatgccaattatccgagcaatctggttcattaccacagtattggactgttgctgaacaagtctgcagtcgattacaatagcatcgtgggagtgaggaggcctttgaagtcaattatttgttgaagacctattacctatatggttgggctagaagctctatgcagatttccctgtcctgtgttctccactactctttgagggactgttatttttttttttttacattttgggaaatggaggctcataaattggagtcagtaaaatttggagccagaatctttatcagatgacttccaaaagagatggctagccataaaaataataagaacctactaactttagggtctcactatgtaaattctgaaatgattagaatggttcttcccaattttagcatagactataagagtggaagagagccagaatgatctagattttaataagagtcactgaaggagccagatcctcacctccaatgctctctgcggttagcattccaaccttatatccatataatgcttctaaatagaatgccaacgtcattgttatattgttatcattataatcactcccctatatttattgagcactcattctgtatctgatataataggattgttagaggaaataacagagaaaactctgtactttaatttttctttgaaatctattttattaaatatcagaatgttttgtggttcaaattatgtatatgaaaggcctttggaatttataaagcttatcatgatttttaaacttccacatgaacatatttgaatatttcaatcagtttgtgagttattttaatacttagtattttttaattacatatattatacatgaatagtccatattatgaattttttttctttcctgtttaagttatcaccccagaagtaaatataacaaataatattcatcaaatctccttttaaaccagttttactgtaacattccctcaaacagaaaatgaataagtttatgatgttttattttagtctatcttctatttttttaaataaacttattttttattggtgttcaatttaccaacatacagaataacacccagtgctcatcccgtcaagtgccccactcagtgcctgtcacccattcacccccaccccccgccctcctccccttccaccacccctattaagtcaaaaaaggtttcatttcccagaaaaccttaagtgtccataatctgggtaatagagattggcttttagcaacactaagctaatgctggtagcccttcctagtgcttatattctagttgattatggatagatagtaaatgaatagacaaggtaatttcagagagtggtatatgctatgaatatttgtaattaaagaaataaaattcaatttaaatatagttagaataaaatggaagtccgtcacattatgcttaatatcacatgtgttataaatatagacctccacttaatattagatcttgtggcatcttcgtaaaataatcatatattaatataattcatttattaataaattcaagcttagaatctatttcctttatcataccatcaatttgataacagtatatcagagtttatacttatgtattaatgtaggtggatatgtgtttaatatctatcccacagaactgtaaattccacaaaagcatatgtttgttcaccactgtatctgtcatgcccaaaacagtggctacctcggtagatgctctctctcacatacaaatatatacctatctgtacattatatgtatttgtggcataaaaagtaattgtttctacaaatattaagaatgtactctgtattgaatattatatttacagtaatacaagacagacaagtcactcctctcatgagctcacatttaaatggaggaatagacagtgaacaactgttctttattatctatccttcaaaattctaccataaacttaaccccctatacaatcctcaacaagcagttaagtactttggaaaaaagaacaggttagtttaagaatcaaaaataataaaattagcaaaactgtatacagaatttgataagtgctaatatctgttttactgctttgtgataaaagctaagttttattatcctcatttcacagataaagggatgtagcatagagtggttaagtgactgcccaaagttacatagctagcaagaggtagagctgaggtttgaattcaagcagggtagctctagcttgcacgctttaacactgcactagactttatctcaatacttcggaaaggcacacataacctttttttagtgtattctttaacagcttatcccactttaaatatgacagatctttctgcataacattattgtagtacaccacatatgattcaattcaaatagaagtctgggtccgaattcagtttcacctctgttctatgcagatgaactaaagtatgtctaaaaatacagaaagagaaaataacaaattctagttttaataggtcaaattaaattaactaatggctttattcctcttctgaactattttatcactatagttttacttcttctcaagagtatagtgatgattacttttaggtcgacctgactggacaatgggttgcctagattttgatcaagtttctagtgtgtctgcaagggtattttgggatgagagtaacatttgaatctacagattgagtgaagcatattgctctttttaatgaatgtgggcctcatccaatctgttgaagtcctgaatagaacaaaaagactaaatctcctttaaactgggatttcagctttttcctgtctttaaactcagactgaaacatcagtcctttctgcttacctttgcactggagctacaccattggctctcctgggtttccaacttgctacctcaccctgaagatcttggggctttccaggcataactgtgtgagccagttcctttatagttactatatgtgtgtgtatatatatatacacacacatatagtaaatatatgtgtgtgtgtgtgtgtgtgtgtgtatatatatatatatatatatacacatcatgttggttttgtttctctggagaactctgactaatataagtattaatttcattttaaatttctatgattttttttcaatcactcaatagtacaaattaagataaggaaataaaaaaaaaaagataaggaaataatctgagtgctaaattaatcctttcatgttcctatctgctattggggctataatcaggagagtgaggagagtgagaagagtgagctggagtcctagagatgaagtaagaaatacagagccccagaagccagaaagacaaaagcaaacagatggacctcagacaaaacattaaaaatccataacagttcaggagtaggtaactaaaatgttctaggaaaatatcatgagaaccagttaaaatgtctccttaaaatgcaatttgttaactctaggttctctctctctccttctccctttgcccctctccccattggcattctctctctctctctcaaataaataaatgaaatcttaaaaaaaaaatactagcaaaagattttcagctaattacctataacctcagtaaaaatagaattcagctttttaccatattctgatttccttaactctctggcaagtagatatgttaaacagaatttaaacaatggccatattatatatgtttctataagtgtgtaaatatgtctatatagataatccttgacttatgatggtttacttacagtgattcagcttaatgaatcttttggcttttcagtggtgagaaagtgatatgcattcagtagaaatcattcttcagattttgaatttggatcttttcccaagctagcaatatacagtacaatactctcccatgatgctggtgtcagggagcaagaatacttgtcccctttaagggttcttctggctggactaagaatccaattgacgtgagacagattaagaggagaaaatcagatttaatagtatacatatgggaaatccacatcgacatggaaattccacagacaggcaaaatgaagtgacatatattatcattctgaactaaggaaaaggggataggggtatcgcgtttcagaggaaagaaaagcatttcgcaggacaataagaagagcagatgtttgataattagatgtttgccacgccatacacattcagatcattcagataaaatttatctctgttaataacccttattctgggaaaaactctcaatttagatttttctatgtaatcaagggaagggcaaaatttctcttgagccgatatggtctcagttgcctttggctcaaaataatccacatgacagagtggtaccttctgaggcacctatcccaaacccctttactgggtagtggcagcaaaccactgctcccagtcagccatgccatgaccagggtgaacaaccaatacacttacaacctttctgtccccatgcaactgttctggtttttcacttttagtataatatttagtaagttgcatgagatattcaacatttattatgaaataggctttgtgttagatgactttgcccaattataggctgatgtaagtgttctaagcatatgcaaggtaggctaggctaggctaagctaggatgtttggtaggttaagtatattgaatacatttattaccaatgatcttttaaatttatgatgggtttattgggatgtaaccccattgtaagtcaaacatgacctgtatgtgtgtcttgataaaagataaactgaagtataataaacattttaatagtttacttgagcaaaatttattggaattaagcagtgccaaactggaagtggttaggagcgctccactgacagagctgggggagagacttttatagagaaaaggtggaagctaaccaaggaaattattgaatggctagagcctaaagcttagtcagctgtttgttattagttgtccataggtttcgattttgtactcttgtggtattttgtacctttcagtttgggtttgccaaaagtaggtcaccatgacattagagccacctcagtctaatagcccccccttgtttaattaatttaacagtctgaatgcattcataaaatttctctcagttcccttgactatgatgtcagaatctggacttttctaagggaacttttttatggtttcagattagttgttgtgttgtcaatccattttcttaaatatttatgaatatttcaataaattgtagtatgtcagaaagtggagggcaccattttgtgaggcacaggtgtctccatagagcacagcttaaacagcttctgaaaataaactaaagatgaaattatggctgatgttgtaatgcatattatccactaagtggctgtttcctactttaatgaattgctattcttctatgtcaatagcatttttgcatttctgaaagagaaataatgtgagaccacccgttcttctgggaaatatgtgtatgtactagagaagataaaacgttttcaaatagagttaagcctatccctcaaatatagataagaggcaaaataatagattttgagaatgggcaaaaattacaggataaaactaaatatgaaactatgtgataagactaatcattaaaatggatctggtaaaggtgtttatatagggctgtctgatattattgtactctttttcctgtgattttgaaatgacctatgctgtatagataccataatttcatctggagaatgtaccctgaaaaatattcttgcctccaaaatttatggtataaattatttacaccaaaaactccgctccccttgaagtacaagcttgagttccagacacacaaatgtcataaattatttgcaagggatgattgtgaaatttataaaaaacaatagacaatggtgggacttgggctaatgagagggagattatttggagaaactaatggaaatgagatttaagaattttatttatgttggagtaatacagtagcatatccaatgaccagaggagaaagaaaaaaaaagtaagaaaaaagaaagatgaagcatgagaacacccacaaggaataatttcttttgcagagaaaacaaatcattggaagaaaagcatattgaagaagtgcaaggttttatatgactgtagcattggcacctttggcctaatacaaaaggctattaaatctatactttaataagaaaggtgaatgtttctcagtgttttcacagcagttctcattgctatgttgcagtctcttcccaagtgtatcatcaaattttgatccacatgtattttaaaatataaattagcatatataatgttatagtgatgactatcaaaaagaatattgagctaggcagcgccaatgtaacattgagacagaaacaacttattgcaacaataagagggaaagaagtaacatcaagtgtgtagcaagagaaggtcaaatatttattgaaacagtgacaattctgtcttccaagtttagtggaaagtgcccaggacagaaaacaattctactcagaccttatgacagtgctgaaaggctaccaaatggatagtgtcatgactgtggatgcttttataaactctgtgagacagtgccactcagcagtcaggattctcttataattattgctggcttgtttagagtgatttgagttttttcattcattctagttggctgccaaaaagctggtgtctcacaatttgtctcactgcagccacataaatattgtgaattctgccttagggcctggatgccaccagcaagctgcccagtgtaaatgtgaagccaagagccaaaagcaaaggagggggcacaggacatgggatctcttattgccagaaagaaggacaagaatgtattagcattaacaaggatgaagcttccaagctgaattatgaaagccttcatttgtaacttaaagggtaaatgctcagccttttgaaacagtgtgtagtcaacaaatgagactgtagccctacaacttccttcttaaaatgggtctttcagtatctctctactcagtagtaactagatgagtaggctaagttgtgtatgtgatatgcctacaagctaaaatagaaaggagtaaagggaaaatgatttagtggctgttatctttgaaggaaatgaaaatttaagagctcccacaacatgcatcccaagtaaattctagaaatcagaactgctcccttgtttaatgtttatgaagagatttttagagtttctgtcaaaataagacaaagtttggtgtagccagtgaccttatgaccatagctgataaacaaccaaaactaaaacaaaaccactagctcaccttcaaattcttagaacaattcattgataccacaattcattgatttgtggcaccataataggctctgttgtgttatactgatattagaatgtcattttcaaacaattttgctaggtcacctcttgcaaattacatacatatttgccaaaactacagttcttcattgtcttcttattttgtgggcagtgtttacctaccttttccataattaggataatggctaattccctcaccttcttcaggaattttctgaaatgtcatgtcaatgctcaatattctgacgctctgcttaaactgccaaccccaattcctccttcccctggtattccaattcacctttatgttgctcaacttttcttttttctgtagcacttatcactttctgaaatactacatggcagtatttattgtctgcttccacactacccaagaatattatgaggacaaggatctttctttgttttgcttttattgcattaaaaatctttctatagttgaagaaagtagattatctaaattagcatatataaaactgaacacccatgctgcaattctgaaagcaaagcaatgaaaaacttataagaaaagttcttaaggagtcaatcaggaaaacatctgtgtcagaaaattgccctgtggatgtaaattaaaatataaatcttgctgtggactggtaattttctgaaaaatacatttggttcattagtggggcactgcaactactcattagctgagtagagaggaatacaccatcaataccttaacccaatgcccataatttacttttcgataagatatttattttaatttttgaaatttatcaaggtaaaagggtgggaggaaaaagggctcttccttgattcaacacatttatttaatttatcatcaaatgccattgaaaatatttaagtataacatcgtaattgatagaacctgtcatcgttccctcaaataattagtttaaggaggccactagggaatatatgcaacatgaaacaagccctctcactgcagtcattctaaaaagttcgatcaccaggaaaacaaataccagaactgacaacacatatatcagacacaattaagtgaatggtagataggccttggggagtatcagtcctaacagccatttcctgcattgactaatgacactgtatcccaaagctgggctgtaagacctttgcatgcagagtaagaacatgttctgtttgtaccccatcatcttaaaaattgtgcacagcatcttatagtcaatcctgaaaatattttaaatgtattcagtcaaaacgcatctctccctacttctaggaataacatggcccgagtgtcttcatcccatgttcctaaaattacagcccaagcagtagatattactcactctcttcacttgagatgtagccttttctgaaatagtatgcattgaaatactaatcttttgctgaacttcccatcctttcaatattttgaatttatgtcatttttcttggcttgacaggagtagctttccttaccaaataggttctacaagggcctctgtccctacctgtaacttctacacctgctggcttctaaattttacaatattcatttgtggaataaagcattgtgatttgggttctgtagcagataaatcttatagaattttaaagcttaattaaagcttaattaaaagctacttctgactacctattgaataatagcccaaaatgtgatattaatttcttaaaataaactcatttttccttactagttgtagtagggtaattacactaattatagcaacagataaattcttgtggcataataccgtaacacatatcacatattcaacatagattagcagggtttggagagggatagctctactacacaatatctcagaaacttaagctaatggatgctcaccaccttataccagcagcacctggaaataggacttctgaagtcacaatctcaggagaacagagagatggagattaagagacttgggctcttcactgcttctattagaaatgactgaccttacttcctcccacaaatcattagccaaaacagtcacatggccccaacccaactacaagaaattttgagaaatgtagtggaatgcatggaatatttggtaagaactatctttgccacatgtttcccacatcaactatgggtgctaacttagaatagatcaaacttcaaaccatgtaattttaataaaaaatacatttttaatgtgtctggatattctgaagacctaagaatggaatataatacatcagtaagaaagtaattgtaaagaataacactaaaatcacaaaattgatctcctctgacaagttaccttcttcgatgctactcatatatcctctaaatgacacattaacttgaacaagactgaattcagctcatacttttcttattgaccattgggtgagtccataaaccgagctacatagtgcttgttcatctgttgtcaattcgtttcaatttctttctttgattattgcccctaagagatgtgttctttcagtttccctcccctgtacaacattatgaagcattcacatatttccctagataaataaatgtaattagaatgattaatgttctatatattaaatattacaatagcaaggacttttcacttttctaggtagaaaaaaagtgatgtataaagttattttatttaatcttttagaaagtaaactttatatttaagaatacttttagacttaaaggcaagttgtaaagatagtacagagtacccctacactccccacccagttgtattttctaacaacatcctacattattatagtatatctgtcccaattaataaaccaatatttatatcttaccattaatatactttattcagacttttagattgccttaggttttagctaatgttcattgtctctcccacaatcccatacaagatactacattgcatttacttgtcatatctccttaggctcctcttggttattatagtttctcaaactttccttgtttttgatgacttctctgtgctaaagagtcctgattggatattttatagaattctcttcaagatggacttgtctaatgtttctctcattattagatggaggttatggatttgggagagaaagaccacagagatggagtgccagtctcatcacatcacaccacgtcaatagaacaaactgtcagtgggactcaccactgcttttatccttgtcagcgtggctggggcagtgttccttaggttttgccactgtaagggtgtttcctcacgtacacctcatactgtcctctttgaaaggaagtcactatgcacaacctccacttaaggagtggggatttatgcttcacttcttttttcaaatcctaatcctagccaaagcctcccaccccaatcaatcatagtgtagaagcagctcacctcagaggtatggccaggttctacaccctacctcctgaggtcaaaatatttacataaagtgtacagaattcttctacacagaataattgtctattctatcaccatttacttatttttatgcggtcatttatttatatcagtatttactcatggatgtttattttatattttgggttataatacaaagtatgttaagtatgtaatttttttgcccaaattattctagctttggccattaagagctgtttcatttgatttctttgtccctttgatatacccaaaattatgtttttttaaaaaaaatacttctttactttctggcactatattttatataatgtcttatatatttcccacccctagcctattatcagttatttctcaaggagctctggttccttttgtaggagaatgatattagaaatcaagatttgagaacaacccattgctgcggggttagcattcctcctaggcactgatagctgacaaagcaaagaaagatgagagtttttcattcagtattgacagagatcaccctaagatttgctgggattcccaacaatcctagtctagaattaaatatccttcctccttatctaaattactttgataaatgtggaaacctataagaggataaaacgaactatcttttaaaaaagtgccttagaataaaagtagggaggtttgaatttatttcaaagttttcagtttaacaattgaattagtactgctattatttcccccatatttctttcgagttgttattattgctccttatattttgtgttatttttgttaacccttcttatatccacttgtgtaggttggctaagactcgtaggccatgaaatggaagagtagactctggtttttattgtactataatcctctccagtaattgaacttcacgtgagagtgagagcgatacttacttatatcaggaggttaatccaagaaccaaaaaaggaatcagaaagagtgaacgctgatgtgccagtcaccagagctgaaacatctgggcaactaccagcctcagcatcttcatttttctgctgttactaaggttggcgaccagggacttactcaaccttaaatgattctgcagctgtgtcacccaaaattgttccacaaaatttctctaactccacagcctgctcttaatgcactctgaaagttctgtgccatcacacaggcctcttcatacccatagtgggaacacctctaataagccactcaggaggacatagcctatggtgggcatcctgagcctattctttctcagagagctcaaggcccagatttctctgtagactgttacgcactgagggttgagctagaaatctagaagtgaggaaataacgtaagtgaccatgtgcacatctcagcatttttctcttagagaaaataaataaatgtgcaatctaaaaggaattctgaaattgttttatgtcttaaagttaagtaactatacagaaaaaaattgatctctgcctttaaaaaaaggaattctaaacaacttataatctcatttttataattccataatttaagcatctatttacaaccagagtttgtagctaattctctgcatagacacaggacagaacatgctgaaaggtaaaagaagaaaaagatagaaaaatagaaaagtatcaattttaagcatttgagatgctgtgtctattcacagtgaaataaattctttcatttaacaaatctttattgaatatcagggtgctaggtactatggcaaaccttgaggatataaatattcatgaaatgccttgaattatatgacaatctggtgtgattaagggaagtgtggtaagtgttgttaataagtatactatccaaacatccactcagaaattaataagtagattatccaaacagcctccaaattgagtcccaatgaaccaaataggagtagccaagaagagtaaagaatggacattctaggcataagaagtgggatgagcagaggctagaagtgcaagagtatatgatttatttgaaaaactccaaatacctttaataatgttacaataaatatatggcatgtcataggagaatatgcaagaagtcagactagaaagataagcttgaagtcagtcataataacaaattaatactcaggcctaacgataatggagaatattaaaggagtatggacagaacaaagatatgatagtaatcaatattttttattattttttaagactttattcatttattcatgaaagacacagagagagagaggcagagacacaggcagagggagaagcagcccccatgcaaggagcctgatgtgagtatccatgccgggactccaagatcatgccctgagccaaaggcagatgcttaaccgctgagccacccaggcgtccccagtaattaagatttagaaagaacattctggctgcaggatgaagaatgtgttgtaagggaacctcagatgcagagggaccatcagaggaatgatgatggggagcccaatagcgatagtgacagaggggaaggtggaaagttgtcaaccagtgagacactaaggagacagaacaacaggtcatgctggctgtgttggcatagggattgaggggacaagaagtttttggcttaattcctgagtaagtggtggggcttttaactggtacagggaataaaggggagagaggagatctgagagggagatatgagctcagagggaggacaggcacaagttgtgctgcagggcttgttttatatacaaatttagatgccagtagaaagtctgacccctagatctagctttcaggaaaaggatccagactgcagatagtggtgtggcagacagtgggaatgggaatagtgaccttaacagagcatacttttaaaaaagtaatagtaatagggaaaacgtaaaatatttcacagcctggtgattaaatcctcaagctctatactacctagttttctggtcttgtttgctatttagtattattatttttgagaatgttagttaatttatttagctttcaagtcctctataaaatgaagataatagttaagaagattacataaattaatacgtgtgatttgctttaaatagtgcgtgtatgtgtgcatgtgtgtgtgtgcacatgcatgcttgtgcacagaacttaacttatttgagactgcttaatatactggttttctttatagtcagtgtgtcagtcagtgagttttagtatatagcgtattggttaaaactataagagaagcataaaacatggtacttgccaagaaattcactatttgcaaggattgcaaaggagtaaaacagcagtgaataatgatatattttccctatcctcctccctgtccctcaacttcatcatgccatgttggatttcatttggctcctccaatgccatgatgttcctttccttagagaagggccttaggacttttgtccctgtttcctaaggctttgacaacccttgacacttggccagcttctaccacccatatctcagctaagactcataggccatgaaattactttctctgacttgagactaaattaggtcccctgttatgtttcccatagccccttatttttcctcaaatcattttattcgagttattcaagaattctagagaaggaaaagacacctgcatgaatgagagtgaccagaaagaagccagtgtgggcaaaaaaaaaaaaaaaaagaaagaaagaaagcatttcaaatagatagcaatacttgggtcccagatattctttttttttaagagtttatttattcatgagagacacagagagaaaggcagagacataggcagaaggagaagcaggctccccagggggagcccaatgtgggactcgatcccaggactgaaggcaaacactcaccccctgagccacccaggtatcccccagatatgtttcagatgttacagttttctaaaaggtttggcaaactcgtagatagaaagaagcttgtaaacccttttttaaagagtgacaaacttgtcaatggaaagtaatcaagagaatgctttaatatacctctcctccaaattttggggtgaggaagttgtgctgtgactatatatactaagggcctttcttaaataattagataaaaaaactttggaaaatttaaaaagtatgttatgatggctcatgactaagcttgctttcttcatagttttaaatgatttttattttattatagccaaatagatttaacttgagtcaacaatggaaaagccactggcaatttttaatttttcctctatcattgtgcagattattattacaaacttccatgtgctttgggggcaggaagggctaatatttttgcatccctgctacatctcaggtgctgtaccatgcactaagtatatgccattttattcactgttcaaaacaacttgatgagatattattttctcgatattaggtaaaacttacatgtttgctgaaattgtatagcatttatactgaaagcttaattttttaagttagaaatgatttggtgccatgaatgagcaaaattcatgaatcaacaacaacaacaaaaaaaaaaaccctagatatataatatcagagtgagttaggtgggctaaatgttctcatgcaactcaaaagaagactttttatcaatatcttttagtgccattttttgttttttgaggaaactgcataccttctatgcactagaattatactgggttttgtgaggaatattgagtagaaataagggaattaatataagggaatgaatattttataattgtacaatatctcatacttttatttttttaaattttttagagactttattcacaacagacacacacacacacagagagagagagagagagagtgaggcagagacacaggcagagagagaagcaggccccatgcagggagcctgatgcgggactagatcccgggactgcacgatcacaccctgggccaaaggcaggcactaaaccgctgagccacccagggatccccacctcatacttttataaactctttctcattttatagctcctcattcggaggtaagcagaacattattttattatttccatgtaattctttattctaactaggaaacagaagcatttggaagcccatgtgttatttctgttttctcctctgattgaagtttgtgattttggaaaagaaataaagcatgtatgagaagtgaacagataaatatactaaagccttcaaagaatagactagttttctgggcttgctgcatgattaaataatgcttatctgtggggaagggatgtggtatcttatgaagactaagaaaaaaaatatttcaaacagttaacccaatgtcaatttaaaacttaaaccttggggatccctgggtggctcagcggtttggcgcctgcctttagcccagggcgcgatactggagtcccgggatcgagtcccacatcaggctccctgcatggagcctgcttctccctcctcctgtgtctctgcctctttctctctctctctctctctctatcataaaaataaataaataaataaataaataaataaataaataaataaataaataaatctttttaaaaaaaacttaaaccttatatttgtgaggtaaagataaaacaataaaactgagtacttgaggttttagagaccagaacactttgggatatttcaggacaagaatattctgggcaagagtacagcaacctagtcacgttagtgattacttctctgtttttgtctcctttaccttttctgttcccatagcttcccactcttggttggtacttttgtatttggctacctcctacaatctgccaaactctgccatccaggtataatatggtgaacatcagggtgatgcttattttccatagtgagcatcggtggtagaagaggcccctagtgattttcaggagaagcaaggtagtatccaatggctgagttgctagcatacataaagcagaggttgtaatgatataaaatataatgtaaaattctgccacagtctgatgaaaaacaatgaaaaagattttactggataagatgcataaatagaatatgcaacagaagaaagataagtaggaagaagggtatggaaatgatatatctaaatggaacatttaaaattgcgttgtctcctctaccacatttgtacatctgatacctcttactctgttggtgcgttttttcttctctctgcttgtcatttattaatatactagccaattttcttatttataattgattctttccctccattatcttctaaggcccataagtatagaaattttgctgtattttattgactgttatatcccaagtatttaaaatagtgcctagcacaaaggagattctcagtatatatttgttgaatgaatgaatgaaaataaaaaagttgtgagtggccgtcttttgaaacatatttgggccagcataagagaagatagagatatgatgatatcagcacatagactttatattgccctgtcagaaaactatggaatatgctttcctaatgagtatataaaactggcaaaaacacaatatatattagtgaaaggagatttcaattatccaaatatcataggaacctgttctttaaacgtaacacaatctacatttctttccttatcctgcagaaaaatttgtatctcagaaagttgaactaacaacaataataaccaaactaaatttgatcatgttataacaataattgtaagcaaataataaatacaatccaataataaacacaaactgatgacattttaactttccagtcacaataagaaaacacgggaagaaaaagaagaaatttttaaagatataaatgatgttttacagtaagttctctgttgtgctcatatttaagagagcatgtaaagaagatggaaagagaggtgatcatcccagtaaggccaaatataaataatgagacactgaaaaaactaaaagctggttccagaggaagatgagcaaggtttctggttgaagatgctgtaaatatgtattatgtattccattcctcaacaaattcaggagtgacaacctccatttcataggagattaatcaatatgtgaacccttgggtgcccatgagagtctggaaatagaaaacttaactgaagggtacatttaattgaattcattaaggatgtctgtcaggttcaaaaagaagcacagatgctgcaagtctgattcatgaaagctccaggtggcattattttacattatctttccatgtacattttagatttaatgtttgcaaatttgttccttatgattcattaattctacccataaaccatgaaaatgtaaattataaaattaaatccatacatacacaaggaaatacacataatcatgcttaatgcctattcatctgaataattccgttgaactttgatctcacaaatcattttcttttacctctatcacataacattgctataagcatttcaagttttcccccattgacttgtcaatcttttccattccatcaaaatgatataaaatacttttgaagaatttattttttcctttctgtaagtcactgaaattctcccaggactgatgtaaattcagttagttgttcttccaggcaacagttctttccttctcaggactctacattcatttgtaaacagtaatacatgcttactagatgctagatacaagaaataatcattatcattgtaattttattaatttgacactgcagtttggtacattaagaaaacaatgaatgtgtattatttattattttaaatacagtgtactcattttttaagcattactgttactagtattttgcaaggaaaagagtatcaccatagccctacacaagaggaagatgctaaaaattttaaagttgtagtaatttgggggaataccactagattaacacctagtggatgctattggttcaccacccaaatactctttccttggcctgtgcaccacccacacccatagctgaagtgcccttatgaatgggaacttcttttctccagaggttatgtaatcatgtaacaggttgcagagtgtccaaactgaaactcagatatgtaaataaaacatttgatttagattttatcttcttcacagaaccctcatcccgaagagggagggcaggaatcatttattataatctagtaaattcattacagtttaataatcaaaacaatcattttcactctctgttggatgactgctctttatatttttccccttgccccttacttccatctaaaatggcctcagtaagtgtgtctatgtcagactcatctatcgagaatcctcaccaccaccacccatgcagtcctgttcccaaggcaaagtgatcatggtgctagaaataaggttggcattatcccaggagtctttatgttcttgggctattcccaagcatgctacatccaatcctacaaagagttgtggctccaaggggagcagggaaggtggcatctcacccatgaactctatagctctcttccttcctgccgtaatctaccaggtctggaaaagcatgtgttcttatttttctttaagtctaaatggcattttatttatgtaatactacatgatcaaatgaccagacctgcttcagatataataatatgggccaatgaattttggaaaatttttttctatttcactaaaatatgtctctgaaaatgaaggctaaacaataatcttgttctgctcttgcctttattttccctgttaagataggcctcagagcagggtaggcttgaaagtagagggtatacagagcataatttaagtagaaaatggaaatacatgcttttaatatattcaaaagaaaatcacatttattgagtactttctacatattaaattctaaattgtattaagccttatgataagaatattctcatataattctaataaaaattatggcataaaatttactttccacattttcaagataaggatatgaagttagtaactttccaaaataacatagctgataggttgaaagcttgaattcaaactcagacccatctgactttaggttttatactattaaccacgataatactcattcaatgtcacatctctcaaaatcagttctttttctgtgaaatttatatataatgaaatatatagctagttaatgtaagagtgagaaattaagctttgtggtcaaatggcttttatttgaatataggttccccaaatattatctatgtgaccttaaaattgacccttatcagtgtgttatcgatgcgtaggaaacaacttccaacatggattagcttaaagcgataatcatttgttatttttcatgatttcttgattttcctgggcgattctgctgaacagtgctgtgccatgctgtattggtgcctgaggcaaaagggaaaaccagtaatactgattctgtctttatttaaaatgttgatattttgttcatcatagatttctttgcatcattttttattttttgaactactgcattattatttatttttatttctgatttttttggaggtgcttccttaaactttacacatactgccaatgtcccgttaactcattctaatctgtctgtcactgagtcttggttttactcacatgtgtcttcggtcacctgacaggtcatctgtgagccggcttgtttaggatattcctgaccaggatggcttgacccagctctgtgcaatctcatgtcaagtgtctcttgtatctttctagcaggacagaccaggtctattctcaggctaatctgggtgtgttctctagatgaacagagaactgagtcagaaagcagaggcatgcatgctcttttgcaagtctctgcttccattaagtttgccactgtatggccaaaccaagtcacaagagcagtccgtataggaggggactatgaagttgtgagttacaggcagagggcataaattcaggaagaccattaattagaaccataatataatcaatctaacatattttacaccttgacttccttcagtgtaaaataggaatactgtggaacctactacctaacattgttgagggcactgaatgaagcatttcagctcttaatagagttcctgacatgtggtaagctttcaataacagctattattttattagatatttgaatgtatgttacattctgaattaacattttggacaacagagaattggactatcctttggtatgagggaaagaatgcatgctctggggatccctgggtggcacagcggtttggcgccttcctttggcccagggagcgatcctggagacccgggatcgaatcccacatcgggctcccggtgcatggagcctgcttctccctctgcctgtgtctctgcctgtctctctctctctctctctgtgtgactatcattaaaaaaaaaaaaaaaaaagaatgcatgctctgttttctaatatagataatagaagcaataaatacagaagatcaacatgtagatttttaaataaagcatatatgaaaatgtaaatgtctatcaaaatgcctcttgtacacaaatattcattattatttgaaacagagccagagtttccttatcgagaagtacatgtggtctatcaaagtgaaattctagaggaaaaataaagagcaaaaataggccacatgagagaagccttgacttttctaggatcagattgtctccagagcatcttattaatttaactatgggcaggaacacctgtaaactaaatttagtttagagatgcctgactggctctgtggttgagtgtctgcctttggctcaggtcatgatccctgggtcctgggatcagtcccacatcaaggtccccacagggagcctgcttctttctctacctatgtctctgcatctctctgtgtgtctcatatgaataaacaaataaaatctttaaaagaaaaaaaccctaaatttagtttaagaaggtaaaatgtgtgtgtgtgtgtgtgtgtgtgtgtgcatgggtgagacagtcaatgctgtgattattgggctcctttcctcttgaggagctgactgatgagtgaaggtggtgaaagatgttactggtggagattttccatcattatgctactcccagtttcttttagtaataactccaaaatacataagccacacaagagaggcaatgactagaaaatttaaggaagatgattgttaaaatgtgcactgtgggtaaatgcttaaagagtggagtaagaggtgttaaattgcatataaaactctatgagaattcttgggattctgcagatttaaagagaaactgggacctaaaccaataaaattctccaggtgaggaatagcccagtgagttctatcaccatttacaacgtcactcccagctgtgtgcttcagtgagggaagagactgatgtcctatgcatcagtgttctcgctcttactttggaaaccaaatattttcaaatgtacttaattaccacttcatgttgggaaaatgcaatcaaatttaaagattaaaatatgctaaatctgcacatgatgacttccttccaaaactgcagtatgaaatagggatagtagctttacagagaagaaaactggcaaatactacctcagcccagtgatcaaggattatatcaatagtgatgagtggtgctggcagtttgtaccagtgatgtgatgtgatgtgacatgacgtgacgtgacgtgacatgacgtgacgtgacgtgacgtgatgtgatgtgatgtgatgtgatgtaacttactccccaccccttcttccctcaaacttataatctctgtttaatcatgagaaaaacattagacaagcctctcttgaggaacattttgcaaatcacatgaccagtaatttttaacgctgagaaatcatcaaaaacaagaagtcacagcctctggagcttgaggagacatgacatctaaatgcaacatactaaaaataaataaaataaaatataaaataataaaataataaaataaaataaaataaaataaaataaaataaaatacatactatcctggttgggatcttgttacagcaacaacaaaaaaggacattaagtaaaaactaaggaagtctgaatacaatgtggactttagttactagcaatgtatcaatattgttcaatagtcaagacaaatgtaccatattaatgtaaggccttaataataggggaaactgggtgtggagtatatggaaatgctctactgtctttgcaatttttctttgaatctaaaatgatttaaaaatgaaggaaatattttgtttcaaaatttttatttttattttttattgttgaagaatatttgacataattgtgtattagtttcagatgtacaacatattgattcaacaattctatacattattcaatactcactacaaaagtgtagtcaccatatgatgtgattaaaatattattgatgatattccctgtcctgttcttttcacctttatgacttgttttataattgaaagtttatacctcttaatcttctttatctgttgctccctttttcccacccatccaccccctggaaaccaccagtttttctctgtttttaagaatctgtttggttttttttgtttgtttgttttgtgttctagataccacttataatgaggggatccctgggtggctcagcggtttggcgcctgcctttggcccagggtgtgatcctggagtcccgggatcgagtcccacgtcgggctccctgcatggagcctgcttctccctctgcctctctctctctctctctttctctctctctctcatgaataaataaaatcttttaaaaaaatagattccacttataatgaaaagattattttattttgttttttaaggatttacttactttagggagagtgagtgcgagcatgtgcatatgcagggagaggggcagagggagaggaaggggatgaaagtcccaagcagactccctgatgagtgcatagccctaatcaggggctttatcccaggacacatgagatcatggcctgacccaaaatcatgagtctggccctaaaccagctgagctacccaggcacctgaaaagattattttaaaagactgattctaacttaatgattaagaaccagaattttaaataaaatataaaaatacgagtttttttatgtgatttttattaaataatcatattatttttttctgtttaccaaggactgtttgaataaactgtagcccaaacaccaatttataggggaaagttagaggtaactaaaagcagttgcttcaacagttcacatctcaagttgaagtattttctaactgcattattattctacagttccttaaacaaaatctaaaacctttttatttttaccatttgtattcaaaagctcttatcttagagtatccctttgtaagaacaagtccttagttatactcatttatacaaacatttatggactataagcagaaattaaagaaagaaaaatactggttttgacgttttaaaaatatcgtttttgctataataaattcaaatccaaaaggatacctctaattgttattccactctgccaatgtagaaactaagaataattattaaatcctataggaagcaatagaaacaagcctataaatattagctgggattttccttcaacagtctagctaaaattaaataattatcaataaaaatgattttttaaattttagtttaattttggtttttatactcagagcccattcacatttataaaacagaggaattataaatgcatgttcataaatagaaaagtatgaaatgtgtgatcatttcagagctcagctgtaccaactgaatggccacatgctgtctttgtatatttccctctagaacaaagtgagtacctgatggatgctttttcataactgtctagatggtaggccacatacttgtggcttttatgtagtacagaaataaatgcacaagagtatattatatttaaggacataggcccttgtatgtgtacagtagtattctgattactataattacctgtgtttgtcttcatggtgaagtttcctgaggacaagaattgtccaatgtatacatagattcgtaggaactgctcaaaatagaaaataaaactttaacaaaaaggacatctatgtttagtaattctatagtagttccaatccaaatagtaataacattagaaataactattggtataggtaataataactattagtatatgcttataactgggtaaacatccattctgttgaaaaatctgaagacagaaatttgtttgggttactgttctgtctgcagcatagaacaacagcatataataggtactcaggaaatatttttcagataaatgaatgcaaggccttggttatagcaaatcagtgaggtgttgggtgttttgtgttggatatgtgagcaaaagtgggatgagaattgttaatgaaccatttcagttaaagcaagttttcaaatccaggacatgatgatgtcaaatctgtgctcctcccatgttactacatcaccatatctcattcatgccagttaagttttacatagaattgaaatcaaatcaatacttttgatcttcccagctacttttttttttaaatgaactgacaaccaaagaaaaagaacaaaaattcttatactaatggaatgatcactagttaggccaaggcctttcacttatatctgcaccaatagagggcaccccacacctatttaaacaggtgagagaaatatgttggcggtgagtcagttaattcttacacgaaggtgtagatgacagaaatgaaataaacaagatattttgttacctgaagctttactgtgcataagaaccctgggagtaatagaatagctctctttcagccattaagttgatgttatttagatgagaacaaaggtagcataaataaaggtgagtcgggattgaaacattcccatctgtttttcccatctcttgtcatttttatagaaaacaccatgtgcagaacacttaatcttaaaagagtgaacaatgagaaatcttcccatagttatagcaattctgttcttgctcagtaaataccaagatcccaatatggcagggtcatatggtgagaatagacttattagaagaggtttgtagaacaaatgtagaacaaatgatcataacatgttgaaattttgcagcagacctgtcctgcattgcctgttttctggatccatttagcagaaaagtcaagtgtctgaactgctgccaattgcctttctcttcctatgtttgcctctttctctccttgattacagacactgggtatctgatagttcattttcatgtgcatttttaagttgtgacttttaaaaagtggagaggttgacttgtattttgtgaacctgatattctcactacatgtggagatcaagagaataacaggacaccacaaatgagccctccattggccagaatgtaaaaatcttcttcattatttcctcaaaatggggcttcatgagaagggttttgtgaacaagatggtacctatgttcaatctaatatttctctgctatcttgtacctcttgccttctgtattagtttcctagtgctataggaacaaattcccgtaaactgtgtagcttaaggcaacaggaatgaatttattctctcacagttttggaggctagatgtatgaaatcaaggtgtaggcagggttgattcgttctcaaagccctgaggaagaatcctccacgcctctctctgagcttcttgttttactggcaattcttgacattccttggcatcgcatggcattccccttgtgtgtatctctgtgtgcttttgtcttcacatgcctctcttacaagaacatcagtcattgagcctaggacacatcatgatacactatgaactcatcttaacttgattgtatctgcagagatcccatttccaaacaagacctcagccacaggtaccaggaattaagactgcaatgtatcttttgtgtacacttcaacacacaacaccctcctcgtgaagcctcattttgaggaaataatgaaggagatctttacattctgaccgatggagagctcatttgtggtgtcttactattctcttgatctctacacatagtgagaatatcaagttcagacaaagctaatgataaacatcgttatgtctggaaaaggtggcggtaaactccaatgtccaagaactttgttttgttaagcagccaaccagtaaattgactggattgacagcaagaagagactgagcaagagcttggtcgtctgacatgttgacaaaagaaataccaagataacagtagggggaaagactcatgaacctacatccactgaggctttttaaaaaattttttctcaattgattgtcatcacagttcacaaaatatgcagtttgctgagtagaatttttaaaaactacacacaattacttgaaatataatagaaacattttatttaatggggcacacattaaaatcacaaagggagggaatcttgcattttaccaaaaaggaatctcccactgctagatgagacagcagatttgaatgctgcaaagaatattttctaaacttttgtttgttttccatgacgagcaatgtatggcttgtagaacaaaagattagaaatgtaaaggttgttgatgcataggcatgagctggatcaattaatgcaaagaataaagaatctttcttaatgagggattatgttaatatgtcagctccacgggtataagctgtgcagtccccatttatagctcataaatgctatagtgacaggtgctctagaaaggaggatgacgaatgagctattgcacatgtgggtaacggtgcagcaagggagagcacattaggccaaatgagcagccagaaacagcaatcaagtcatgactgagggagactctagttcacacaggagcacatctttctggcacagccactgtatttactgacagaccacacatttaaataggttgttccagagatccattagagatttcagtaagacaaataatgatcatgtctaggactgtgtctcttcctgtttctcattgttactaacaaaaacttgcttttcatttcctactgcagttcagctgtccttctatctgtaatcccaccacgtagctagagccattgcttcttttcctttcatgtcatgtattttaccatttacactgttattccatcatacaaaacaagaaaaatggtgccctatgtgttattccaccaatgacctcttgaaagataatgcagcttggttatgtaacaggaatttgatacttcatttcttgttgaggagaaaagtgtgtcttccctggtcctctccttggttaaggttatataagatcattcacagttttgaattataaaatttctctccttggaagtgacatgtaagaatctatatttatgtgaattagttggattagcttcagtgccagttctttgcagatgaattatttgattatggtgaggaagaggaagcaaatcaacccttcattatttttcctgtgtgtatgtgtgtgtatatacaaccacataatagtttaatatttatatgtatttgcaaaatgattgccacaaaaaaatctagtaaacatccaaagaaaaaacctctgtttaaagcaaaatgagctagaaaattatttaataatagctttatgcttaagagtctgtgtttagggatccctgggtggcgcagcggtttagcgcctgcctttggcccggggcgcgatcctggagacccgggatcgaatcccacgtcgggctcccgggatggagcctgcttctccctctgcctgtgtctctgcttctctctctctctctgtctatcataaataaataaataaatctttaaaaaaaaaaaaaaaaaagagtctgtgtttatgttccaggtccatgaactaactataggtgtttcctgagacaaagctttcaagtatcttaatctgtaaaatagacataataatgtctatgtcatatcttgtaaggattaaatgagatcatttatgtacaatgtgtaccacagtgtctgtatattaagggcttgataactgttacaacaaaaagaacactaaaatatttccaaaataaacattcaaaataatttcatcaattctacccatttaattaaaaataatattaaatattcaaaaatataattttccatatgatatgtaggaatataagaaagtataatttgagaactcaaaatatgcaagactgttctaacaatcaaaccattaaaacccaacatgggtttctttgcaaggagtatgttccctgtcactggaatgtttaaataaaagtggaagggctagtatgggatgaagcccttgtgggcagaaggcaggactaaatggccctgtttcctctcatgattatcctctattccagggaagccaagcctattgtcttgtttcttatcatcattttgctttcattttctattttgtttttgttttttctttaattatagtttaaaaatcttaaatgatattcactcacctaaaaatcctaaacatcatctatttcaggcagtttaggcaatcccctctgtgataccccagccacttaaagtgcatcaagctgatcacagcctcaaatataacacttcaagataaggaccaggctaacttgaaagctctctgtttatctggagagcaaaccaaaactcatttcagcattcatcactcctgctcaatctcctctgcctaactgccaatgtaaaacaaagagaataaccaaatcttcccacttgaaattttcagagaaactgcccaatatatattttctcctgtaggaaggattatgtagaaaggagaacctcagagtcttcatagtacagatggaaagttgacaattatggaagatggacatgagttattcaaggtcagatagtttaacagcagtgtccttgctagaactgagttctgacatcttgttctttcccaggctccgttgccactgattatagagagagcattgctcatcatctccttctaaaggtcccatggcccatttttcccctataatgatatagctactgagtccattccctcccattagttataagcttttcttaaaaaaaaaaaaaaaaaaagatttatttatttatttattcagagagagagagagaggcagagacacaggcagagggagaagcaggctccatgcagggagccggacgtgggactcgatcccaggtctccaggatcaggccctgggctgcaggcggcgctaaaccactccgccaccggagctgccctagttataagcttaatttcaccagaaactatatctactaacaagaagataaatatggaatctcctttaggtctgcattattttgctgaaagctggaaatgcctttgatggtattaactgtttaagtggtatctctgcatcttatttttctttagttaagccatatgactgctttggtctattcttttcatcttcctcctgtacaagtctcatagccactcttgcagggtttcattgacttgggagtaggcttgcattggcattagctgtattcctgtggatcctgagtcttattacctaccattaaaccagctttactcttctaataaataaaaagaagtaagctacacttatgtgattattcagggcctaacacactgaatggatttcttttctccaaggaagccaccatctagacaatgcacagccctttctcctttgagctatggtcggtgaatctactgtaactcactggctcagggacagtttatacatcatgaaggaattgttggctaaactcgctctggaaagttagaagggaaacaattgggacattgtccttccaatttctgaatgttttaagattatgtggaaaatttgaaagtgtttcaattttccacttaaagtgagaatcatgttttttgcattttacctaatttattaatcttgtgtttgaatatcttggtgctataactggaaacacagacttcaaagccaaaacttctttttccattttgtgcacatgaaaactattcttacagtttgctgtataggcatcaatgtttattaatatttaccatttatttgtctggtacatgatgggcactaggctatgtatttgacttattgaatcctagtaataactcttaaagaaaagccctaccatccccaattttttatagattctttaaaattgagacttacaggtgctatgtagtttcctagaatagactagagtaagttaaataactaggatttaaaacaagtctgagattaaaacccataataccctttttactaaacgatgttaaagacactgtagttttctttttgctttgcagactgccccaatgcctttttgcctaaaatatttcttgctctttctgatagttgtatgccagcttggcccatctggtgctgttttcccagaattttacagtactgcctttttattcaaagatatactttgaaatttccttaaaacattacttatgctcacctttgcaatggtcactttaccatggtcaccttgctgcaaactgaattctgatgagctaagcatgtgggcctaaggatgtcagcaggataccaagcaattgtgtggataatggatactgacattcatcctctacacatgctcagctcagcggagttctggtgcagcaaatgctgttcactgctagcacaatggctgcattccagaactctagtggtagactgaatggtagtgcaggtattttgagaaactcttacacctcaactagctaagaacgtgtaggaccatgagtccttgagaggactatgggatatgttgataatttcactttgatgtataaaatcatggattgtcttataaaatcaacattttcctaagtattcatgattaggcacagaaagttaaaagcacacactatatagcagagaaccctcatatccacttagagatgtttactttcaatactaaaaactcttattattttacttacttagaaggtaaattactaatgttttgctttctttaacatataatgttatattaatttcatatgtacaatatagtgattcagcacttccatacatcacccggtgctcatcacaagtgccctccttaatcctcatcacctatttcatccatgccccccacccaccacccccctggaaactatatttgttctctataattaagagtttgtttcttggtttttctttctcctttttttcctctctgctcatttgttttgtttcttaaatttcacatatgagtaaagtcatatagtatttctgactgattcttttcatttaacattatactctctagctccacccatatccttgcaaatggcaaaatttcattctttttttgtggctgaataatattataatataataataataatatatatgccacatgttctttctgatgttctgatttatatagacaaggaaaattgagattctttttggaattttaaaaatcaagtatagcatgaaaatgcccacattgccgagacaagactaaaaatataagagtaatacctatcaaaagtataagagcaatacctatcaaaacatcataacaatatcacaaggagcttctccaatatattaccctatgttatctgcaagcacctttagcattaacaggaaaggtagttttgtttgctaatcaaatgtggtcagtctactggaacaggagtgtgcttgcttccacacagcttctttggtcaaggctagaagaacagaaatccacccatcatgagttaggttgacaagaaaaatcttatgtaaccaaactgctatacactttttaaaaacaacatatagattatggcgtaaatagagattttaaagtttggtatcctccctcccatagtagtacacagtgtcctttttagaaaatgtggtatttaccttttttgatctcatcatcatgagtcaggtttctgacattgtcccattatatgttggaagaggaagagacctcagaaatgggcaagtgcactccttcactttataagtgaaaagcctgaagtccagatgaatgattatgagtaaaacctcataattctcattcattatttcttttatagagaaagcttaaggccatttttactggaaatcaccatagtccaagaatgtgactggaacttattaaggggcttcagggtcccttaccatactgcagtatgctgatttgtaagaaaatgaatgaaagaaagttgggtcaccacagcccttcttgaagatcttattttctactcaagaatacacgtagataatttacaaaaccatacacaagcaagtacaaatgataatggtgggattgaaggccgaaaacaataagcagctttggtcacttagaacttcttgtctgttggttaagactgttggcagggacattgaattccatgactgtgcacatttacaagtcacctgtgtggcatgtaactatgcagatgattatttagtgggcagtggaaaactggaaaaggtctgaggaggccacagaccaagacagcaggcagtggggaaattaacagggccaagcagtgaaaaagagattattttcaccggtggtgaaaggaaacaaaggtgactaggaaagtctgccttcacagtctaagttctcctttttttaagaattatatttatttatttatttgatagagagagagagcaagcataatcaggggatgcaagagaaagagagggagaagcaggctccccaccaagtagggagacagacatggggcttaatcccacaaccctgggatcatgaccagagccaaaggcagacacttaaccaactgagccacctaggcacccctctaggttttctttttgttgctctagccagctttgctcaaacataccaaaccttcatgaataaataatcaatgaacaattttaaagcaaagttatttgccattaaccaccaacaaagtaactgttcaatgtattatatacataaggagggaagactgaaaggaaaagtgataggaggtttagctctgcctcgtctactaacacactcttggaccccaaatgagtcacctcttagagactcagtttcctcatctgtaagatgcaaagatttaactagatgatcactaaagaagttttattatctctatatttctgaattgagaaggaaggtgatattgatcaaatactgaaaatatgtggaggggaaatagtgatactccttaaatctctttccccagtttttcagcacttccatgggcttatgtggggaaaaaattatttgccagtaactgtaaaaatctaaataaaaatattgagtggaaaaaaagtatcaagtgggagatatccaaagatgacattataatcttttattattgacagtagtatattgatcattctatagtcagtgtacaatatttaattaatgaagtttgattggacatactgtttaataagactgtgacagcgccattgctgttgtcagaacacaaagttagcatcgccatgtctggattctcatgctaccaagtcactctgttttgatgagggccatttcattagcttaatcagccagtcagggggaaccaccccctctgccccacattaaagcattaagctaccaactctgagaggactgacagattcatttccctggtgagtgaaatcatatttgttttagttactcttcttttatccaactaagaaaatttctcctcttcttagaatctccttaatcttcatggtaacaatctccctgcctttttgtgatcacattgatttctatgtcaaactattattttgaaatagattattttgaggaaaatctaaagcaaataaatggttctttaaaaagtcagtttacttcaggtgaaaaaggaaaccaaaggtttcagatggattagagaaatgtgtctcatttcatttatgttctttacaagagagagaaagtaaatacagcccctgcagtgcaatttaaaatattgtcaccccctgccttaccctccacctctggctccatccaactggatcccttaagaatgggaaactggttaccctgctaagtggtatattgaagtgtctagtgaataaattggaaaattttataggaaggcctacaggtacctctttatgtaagttcagtcaccaggaatcattttatggtgattggtatttcattcctttagttcctatttatttctgatgccttactgatgaaataattagaaagactacaaagcaagtggagatagaagttaaatatataattggggtgctatcacccactcccttgtaatctgtcctctcttatcattcattcccctagattgttctctcaaaggttatcagtaaccgcagtgtcaatcatggtagctcagttttgcttctggtgggcttctctctatagctcataacatggttcaccctccttagactctcttatggcagcccaatggcatcatgcaaatcaagttttcctccattatctctggttctcttactctatgctctttcccactccttttttttactgcttaaacacaggcattgccagtgttttgtctttgggccatattttttttctactttctctaacactataagaagctcatgcaccatgatgtatctctcatcttcatatagatgaccccaaatatttgcctctcaaactctccattgaccttcttttccaacttcctgctgaataaattcatcaatctgagcttttggcatattaaaaattagttcaccatctccaccaaagcaactccttgctcctatagccctttattttactgatgccatcatttgctaggcatctgaagttgaatggatactgttgaactttctttgatttttgcttttcctaccaccagtctcaaccattaaaaccatactgtaaacgacatactgtaatttgcctttttcaatttgtacttgccttgccaacattttcttcctaatggatatatagtattccaaaatattggtatatggtaatttctttaatcactatatattattcccaatcctttttttattgtaaataatgctataatgaaatcatcattgatacgtatatggaatggctgcatctaaaggtatgcacagttatttatttttttattttataatttttattgatttatgatagtcacacacacagagagagagagaggcagagacataggcagagggagaagcaggcttcatgcaccgggagcccgacgtgggattcgataccgggtctccaggatcgctccccggggcaaaggcaggcgctaaaccactgcgccacccagggatccctgcacagttaagttatgataatattgtcaaattcctcctcaaaatggataaatttaccctgttccaaaagtggatttgttatccatatttttcctagtgatctgtaatgctattttgtgttctaatgcatacattagtttttggggcttagaatatatttctattgatcttcactgagacttatgccagtaccatacaatcctatttaattttataatattttctatctacttaagcaaatccttccttattagtctctctttttcaaaatcttcatgattattctcctagtaaaagcttagcatttgtaagaatttataaggcctcctgcacggacagctagtaattgatattttaacttacctgtattaaatttatacatttttggatctttacaatattgaatcttttaattcaggaataatatacatatctcaattatttgttttttcttaaagatttatttctttatgtgttttagagcacaagtaaggaggggtgagggagaaggagagagaatctcaagctgacaccatgctgaacatgaagctcaatgtggggctcaatcttaagactcggagatcacgacctgagcccaaaccaagaatcagatacttaactgactgcaccactcatgagccccaattatttgatttttcttaacatccttcttttttttaagattttacttatgcatgagagacacagagagacagagagagagagagagagagagagaggcaaagacacaggcagagggagaagcaagccccacgcagggagcccaatgtgggatttggttccgggactccaggatcatgccctgggccaaaggcaggtgccaaacccctgagccacccaggcatcccttaacattcttcattaatgttttataatcttctccacttaggccttggacacttactattagtctcatctttagcaatattgttatttacgcaaatgcattttttattatattttctaacaagatattattaacgtatcaggtggcttttgatgtcattagtttaattttggattttccactcaacctaactatcttatttacttgttaattctcttgggcttatatatttttcatcaaaagatttggtgttattttaatttatcttagtatgagatatagtacttctcaaaccataaacacaacgccaagaatgattttaattttggaagaaataaatcttcaaccaaaccatttatgaggtctgttcttatgctctgtaccggattaactgtcccactttcaggcttccatccacctgtggtgcttttatcctcatttctccaattggcaatttctgccaattctccatttctatccaacattcaagttctttcttttccatgaatcctttcctgatattcttaacatcactttggtttttcctcctctgaagccattgatagcttcccccattgatagctcccatatcacttcatatacaagtcatgtgacattaatcatcatactgtatatgaattggtggcaggggctatctcttgtcatcttcatttcttctgtggtatctactgctcctttgttgtatgttgttaaagagctcaatatacatgtttttattcactctaatacatcaagtgtctggaatctaggatcttagctgggagggttccatattctattcatcccactgatataccattaatcatgagtggatgaaagtttggttggccatccatcactgatagggaattagatgatgaggtcaacagaacacaggcttcttatatactcataaaaaatgctatttgatttcttcctgatgaatagtccttctactttaccttaagaaaatgcatcatcacacagcaccataaaaacaaaaaaaaatctgaaaaatattaacctatttatagaactgaaattgtaattgcattcccttctggctgttaaagtgtgctactttggggaaaaaatgtccattttatttttataatttaattttatatcttataggtacataatgttattttatattgcagttttaactagtatgtaccttggagttcacttatgaaccttacaaaatacatttatattaaaagctataacagggtatcatttttcacaaaggctctgtgttaaatgacttctttactgaaacagttttttcctcttccttagggatggagttcgactgtttttttttttttttttcattagagatgagattaagttaaaataaatattcttaagaggaaataaatagacaaaatactgacctatttttatttatagtatttgaatgccatctgccttttaatctggtggagtttttttttgttttttgtttttgtttttgttttttactcaaaaatatagggaaagcattttcagtctaaaacaacaggctctttgaaatccagaagaatctaaatagctattgccactcttcatcacaatgtttccacattgcataaccacctaaataagaagcaatgccacaaagcatacaaacaactaggtctgaattcaagcaaatgtgacaagtagtgttataagccccgataatggtaaagcctgtttcttcttaattaaattgtacagtttctttgagatctaaagagagtttcagattggttttatatgccaagctattcttcatttgcaacagtctcctagctaagacactggaaaaaattaggaatatttctctatgaagccacacagaatccctgtgtaaagccataacctttatgatacatgaaccttaggtatcaacaagtaattcctaaagcactatggaacctctctgcaaaaaaatgcattcctcagattggttggacacttcctgagatagatataaatagttgagcatccactgagttttcagattctctgccagtgttttcatgtaagtaccaataatttctaattgctgtactgatatgatctttcttcagaaatttttaattttataatagtataagacatgctggaataatgcactttaaactaattcataggcttgtgatcttctggaaggttcagtagccctcttgtcccaaaggtcagcatccatttgttttgtggcttgctgcttaatcaaacccaagtgaagtgcaaaataccaagagatgaggtgtttttccagtttttttcctaacagaagtttatttttcttacccatttgttcttctagaatgtcaatatgattaaaattcatatatcctgaaaattatgataccaatcaagaagcaacttttttttttttctaatttagtcagccacttcaaggagtccgttcaaacacagtacctttaaccattcactgaatggtttactctgctattcctttttctaacaaactctttctcttccttctccttttttttttttttctttacaaaacagaggtcatgaaatcaggaagattttgaatgagtacttggttttgattttaggaaatttttattaggcttccttttaatgcaaggaagcaaggcaagagtttgttacctagaaaagtaatagttggaccaggagtgagtaagttaactaataataatagttaaattatttaagctggctttaatagacatcatggaaagtagttaatgtgaaatttgattaatgtaattccttgaggatggatgaaggaaaagcagtagtttgccctttgtatatcaagggcttattttttttatcaatggtacttcattagtctttaatttagcaatagattggaacttcaagcgaggacacacacacaccttgtgccctggctaacaaagccaaaataaatcatatgaaaacatctccaaagataagaagtattaatcattagccaggatttgagactgggaaggtgtggactcaagacccatagtccactcctgaagcagcatatcttaactggacattgagaacacctgagttatagcacacttgtatttcagttaatggactactgaaaacacacagcttttctatgacaaactttctttaaatgaagatagcaatgttctatcatcctacagggaagttaataaaacctgtaacaagaaaaacaaacatgcatcaagtgtcttacctgaactatgaagcattctaagtgtttttactcaatttagtacaattcattaaacataaattgagcatttattgtattgtaggtaatatgctttctgctagggagaaataaatgaatggaaccaaaatgttgccaccaaagaggtcacagttgcatgagagtcataaatgtttactaagtagttaccacatgccaaacactatgttaagtcctttacacacattaaattatttattcttataatagttacatgatgtagatgctattacattccccattttatagatgagaaaaccaggagacagagagcccgaggaaatcacaaaagttattcaacagtttaaagccaggattcatgcccaagtttatggctccagaattcacactcacaatcacttcaccatctctagagaaataattacaatgcagtggtggcaggatggttctggaaaacttagaggaggcacagagcagcaccaaggagggagtgatagatttaatcacactagagagggtggaagtaactattcagagaaaatagcattaaattcagtaaacccacgaggtctttataattaaattatcctaatgttgtcttactgagatgaaaaaggtgttattgtactgaaagttttaaggtcacatagctcatatataacagaattaggcctcataacccagactatctgcattccactcatatgtgctactgcttttggcgtcaaccttagctcaactgtggcttgtgaggaaggacgcaagcatgaagcatgaaagcagtaggcatgcagcagtaagcatgaaaggtaactggggaaataaaaggaagaggatttgggggagaagtgaaattcaaattaagcagcagtgtagaaacaagggacttttagggtgactctgaataagatacaaaagcataatagttagctgtttgcaagcaaccaactgcagagacagcattagagaaagcaagagttggaatgtagctggccgctgcctggcatagagggaagggcgtgctgctgaacagggaagcacgtgagtctcagagagaaaaaaagaaattcacacaacctttgaaataacaattccatgtccgcataaggtgcacagaggataaggtgaaatgtccacaccacaggctatgagggaggggtgggagcatcaataattcattgagaattaggtcaaaagatatttactgcaattcaaactgcctgcaagaaataatttgatttaaccttttgggatggaatgatacatgacaaatgttaagatatgtatacaaagaacccaccaaattgctgggaggcgggcggcccgggtggctcagaggtttagcgctgccttccgcccagggtgtgaccccggggaactgggatcgagtcccgcatcgggctccctgtgtggagcctgcttctccctctgcctgtgtctctctttctctctctgtcatgaataaataaataaaatctttaaaaaagaaaattgctgggaggcacgtacagagaatctgttgaacaccaagggatgaaaacagtatatttaatttagtcacaaaatgtgaagaacacagcaatatgactgcacaaatggcactctcatgttgtgaactcttccagcaaggtttcaaacagatttggatctacatggaaaagtcaggtgtgcaggagagttatacagagaagttgcttttaaaaaagatttatttattgattatttatttatttatttatttgagagcaagaaagagcatacacatgaatggggaaaggagcagaatcaagagaatcctcaagcagacttctgactgagcatggaacctgacctggggcttgatcccatgactcactagaccataacctgagctgaaatcaagagttggtcgctcaaccaactgagccatccaggagcccctgaaaagttgctttttaaaaaaaatattttacttatttattcatgagagacacacacagaagaggtagacacaggcagagggagaagcaggcttcttgtggggaccccgatacaggactcgatcccaggaccctgggatatgccctgagccagaggcagatgctcaaccactgagccacccaggtgccctgaaaagttgcttccttttaatggccaaaatatagtctcaaaagcagactggagagtataccagcagacagaaatgagcttccaggttcaccactgggggttctgtggcagttgcagagcagaatctgacagggtcctgttggctgcagcagaagaactgacaagtaaccaggctatgggtggggtctgtgtttctagagaacaaaaagtacagggcactgctgaagaaacaaatgagaccaatttgaatatgaaatggaaagttttaaaattgcaggggattgcagagaaaagaaataaaaatcagtaaggattcttccctcatatagcatgaaaatgctagaaaatttcttatgcaggaggcagagaggataaaactgttactcatgataaagcaagagaaaaattggctgcaaaataattaaagaaatattttgaaaatgataggatgcttaaattgtattgagaataggaacacccaaaagtgatggtggcaatatagaatagatgagaagccataccctttcccagacgtttgtgattgtgaaggtttctgtaaacaaacaaaggaagatgttgtgagctaatgtctgagatttaagctcattgaaaggagagccacccttaccagactgttctctggaaacttaggtatgacttaaagcttcttgtgtttgatgaagatcagatgctctgcctccttgggaactggatcaaaatacagagaaaagggtcagcccagggttttaaaccatatccttctctatacttgaccttgaaaaaatgaaaatctgaaaatctgaaaaacaaatcaaactagaaagttaagtcatctgaaatacatatgagtttgtattctaacatttgctaatgtaagtgaaattggaatgaaattatttctttggctattgaagatgcctagacaactaattataaattaacttagaagttggcataatgagaagcatattagtaaaaatgatgtttcgggcattatttgactagtgtaactagctaaaagtaccttgataaatattatacaaactgagaaaattccagtagtttattataagttagcaattttataacaacgcagtaccttccattctagatactagaaaatatttccagattcctaaagttttctttgtaataacattttactgaggcagatagccattgactagaaaacaacccctatcattaaatttggtgtccaaatttatcagagctatgcatacaaatgtaggtagattctagggaattatattattgaatgaaagactctctcgttactatcagtggactgataaccatcttccttggcagctagagtaaagaggcagagagtgggcatgtttgcctcctgcacctcaagaaaattgtaaatagggaccatttcagacttcctcctatcatgctaattttcagataaaataacatataatcagaagtaggcaaaattcatcatttctcccaactaagccatcgtgtaaggctgtgtgtgtggagggccagttatttgtataatggttctaggtgctaaaataaccttaataagttacctgttgctcttggcattgctgtaggtctggaaataggaatagataagggtgattttcagtaagattgtataagaatatattgttatattagagaggacgagaagtgaaccctgtctcctaagtcagagagtgatcagttttaaaagattttactagctgtgtgatcttggggaagttattttatctccctaagctccctttcctccttcttagctctttcttttccacctgagtgcactttctgttgaatccctgctgtggtcagacagtgctatcccacagacccttgtaagggtgagaggagcaggtgtacattaggaattcaataagtagctactgttacccttgatattgttacagtcaagttagagaaactggagtttagggttagggagttttagggttatggagtcagttctagaatagctgaggaaaggaattttggcagttgtgtcataaaatatgcttagaaaattcaggaattacctataagagtcaggagtgggaaaattaaaatgagttagctgtggaagtgaacagtcagaagtgaacagtctccactattattttattatatctttgtcctatgaccaccacagtttctgaagtggaataaaacagtaacaactaaacaaacaaacctggacataaaaccctgtttgcctcattttggaagaatattcacttcgagaaagtcattttggccagtgcagcttggcattttaattaattagataatgggaaaggcagaggtggatgtcagctcattcattgtagttaatttccgaacatgtccacgtagccttccagccagtctccacagtcctcacttgggaaatctctcacaaccccaccatattccccagatacctaatattagcttagagaaaaccaaacagctggtaaggaaaaatcactaaaatctagttttccttttcttggacccaaccaagagtgagatatgaggaggcaatatgattttcctaagcctttgttttctctatgattattttaaaatgttttctgtagtgggattctgaacattcgacaccaagtaacttgtttgcagcttatcccacatgtgcctcggcacacaggtgtgtagtgaagctaccaaatgatggacaaccagatggaactttggggtgaaccatccatgcaatgtggtgtagaatgtgctatgcattttaaaaaggaagtgacaagaaatatagaataactccaacgtgttactcaagaaacacgaactttctcaatatctaggggatcactctgtgggcaataaataatgcaatcaagtaaattagaactccacgggacactgaaatattttgtatcttttacagaagatgaaattgttcctccacatattattctattaagcaaaatatatcagaaacagaaaaaaattgagaatcactgatctttttggtagaaattctgatgtagcaacatagatctatgataaaagttatgaatcagccaaaatcatttattaaaacaataaggtcaatttggctagtattttcccatctaaagctcacttacatttttacttttttatggaagtaatcttcaaaaaatttacagtgcagattttattatgatctccattttacagaataggaaacattaatggatttaagtgacttgcttaaagttggtaagttgcatagttgaaatttagacatgactgaacccagagcttgcttaactactaaaatctactgccttgagtctctaaactcctctgccagagatatttttcatggatatagatgatcaataaatagactctttaatggtgtggaaattccaggaaatgctaaattcttctctagtggtaggaaatagtcattatgcattactttaaaaagattttgaattcacttgttctttttcatattattcaaaaatccctagggttacataccatctagcttattttggggtgattgatattattaatattattgatattcacaaatttctgacatactggataggaaccagggggatgagttatagtcaccataatatctcttacctttttcatgttcttagtctctagagacagcgtaagtttatctcattacatcaccggactccaaggttgaaagtgagattgccagtgatggactgatagtagaaaacctccagcagagaattctttggtctcagagtttgctgtgcaacagagaatttttaccagtaatgttacatgaccctaagtctggcatacttttaactatagctctcctgttatgtcatattgagattctagtcaacccctgatagtcttcactttttcatttatgagggtttcatcttgataatgtatctatctaaaagctgaaacatttatgtttctatgttcattggtagcctagaagcctaaaagaacaaacatggctctctagttgtttaacataagagacactgggtgggcagagaggatagcactgaggggcagggaaggatactgctcattctaaacccattttcagggaagccaatggaaaattctccaaaatacatacaaggaaatgctaacctcacagttacttctataatgatgggaaagtccatctaatttataatttcaataaattatataagtggttttggagataacatttagattgtcattaaacatatttagctaactgttgttcccttttattcaaattgggaatgaattatataaaatatttatgtgactaattcattcagattctggatcaaagacaagttcttcccaagagtctggatgaagttacagttgccataaaagcataccagtccctctgtttcctttaatattgtggtcattaagctcattagggagacattagagcatgagatgtgcaggcagatgctattgttcaaaactttgatctgcttcttactagttatttgccccaaagcaggtattgtctctgtctcagttttctcatctataaaatatagtgctaaaaatagaacatgcttcatgaaaaatattgtgaggactaaatgagaacacattacaagtcattttactagtagctggaagggacactcaaatgttagatatcattattatcatttgttttatttggcttttctgagaagatataagttccataaagactatttctctttttgccctttatccttattaatgaacacaagtctagatgtgacaaatgatcaatcactatttattaaatgaataaatgaatgtgcaagccttggtataatgagaatggaaccagtgactgattataaccactaatgatgtttgatagcttctatcatttttctctgccaacttccttatttagatatgctattctgtatcatacactttttcatgagatctcttctcaaagacatttttacacaatatttcctccccaaacaagggacacaatctcgttagacattaattcattcagtaaataattttcagcacctggtatatatataaactagcactgagaaacatttaaagaaatatctcaggtactcagtttgtccaaagatctaaagtatctatagtcataaacagtgaattagaggtattgctcattgctgcctcttttcagtattctaaatacagcttggctgtctctggatctatacttcaatgccacagggaaaatcgatagtaataacacaagatgctgagagtagaggtaaggtttctccattttctaaactaatcttctagtctgagactagaactgggaaatattgaactatccaggatttgggtaaatgctggcaaagaatatacaaaaataatatacaaagagatattttattttagtcattttaatagaatatatgatggagtgagtacttgtagatttgacatcctaacatttcttttaattttctagccaaagtaaagtgtcaatttggcagttttctcctttgagtgttaatatgtctaaaatggaaaatttgggtaagaaagaatccacgcttgttcccttcacatgccacatcatattttaattgtcaattacaaattgtatttgattcttacaagagtgccatatgcttcatatgaatgtcataaaatttcatctcctgagttttactcaattttataatacttttgcaaagcttattctttagaataatagtgttaataaagaagttatttgcaagcttagtcaaaacaggcattttttcatggctgtatttttttaaggattttgtagtgttggagtttttatatgcaatctgaatcttgcactttggcacattcaaggtaggattattttatagaatgcaatatttaatttatttctatgataacaataaaattgatcatgtttcaaatcagaaaatattgtgaatcagaaagccaaattaattagtcttgtagaaatcatcaggattttgtgtatttaatcaggactgatgcatagagtcaaagaggaagagagtccttgacttttaaaaactgctgtgttaagtcaggataggctggctagctgcagtgagacacactcaaattctcagtggcttaaaaccactctcacacactctacatattaaatgtagttcagcctgggggcctctctcatcaaagtcactaaggaacccaggctggcagactcaatcacagctcacatttccacacttcctgcctcaagaggaaagcaatgtggtgaatcacatcctgattcttcaagcttcctgacaaagcaagccacatggccaaacctaactcaaagaggacagagaagtataatcttacattataatgtgccccaaaagaaaaggcatacaaatatttatgacacaaattagctgtaaaaaatgttcttcactgtggtcattagtgctacagtttgcatccttttgtcatacacccattattactgataaaatcaagatatttctaaaaaaataaatcaaaagatttccatagtatgacccactctatcttactggttacctgattaattcaaggagcaggataggaattcttaagaattcagtgattttaagactaatggtagctgagttttatttctttctttcatgtcttttctgacctcatatcaacaggatcacatgaattcccatactgagtcagacccatggtcattccagccaaaaattttatccttggaaaaaaatgagtcacttctattatttctccattgttcttaaagaaaaatgatagaactattataagaagtgctattttaaaaatagatgaatctagttaatatgcataaagatgaatgtgcactttctaaaaatcaaatatttctttcttgaatttcaacaaatcccaatgaaaaacatggtatattaagattcaaggttttaaaatatatatatattttgttattattcatgagaggcaaacagagagagaggcagaaacatacacagagggcaaagcaggctccctgtggggaacctgatgtgggactccatccgagaacccatccgagaaccctgggatcatgacttgagccgaaggtagatgctcaactactgagccatccaggtgccccttaagattcagttttaaagaaatatttctaaaatagttttgaaagttataatagaaaaaaataaagtagatacttggaacaaaagtaaaaccatgaaactctagtgattccatttacccaattttataattacaggtcaaaaattatggaatttagttttagaatcccaacattttattttttttttaagtagtctccattcccaacacaggtctcaaactcaaccctgagatcaagaatcacatgctgtatcaactgagccatcaaggcacctctagagaattcccttaagaccatacaccccagcccaatcattaacaaatgaagaaactgaggcccaagaaatattttgctgaataggatagaccaaatccctgccttcataatattaaattcaataaggaaatatatcaagctatgaactctacaactgattaattttaataatatagacaatgtgtctgcaatgtagtaaggactcaacattattgttacacctttgaagtagtccttttattccaacttttctcctaatatttttttccccaggtacacctggcatataaatatttagctactgattgaacactaccagatctatagagttttcaaggcaattagaaagctttcttgattatatttatgaaatgtgttgaatgaacttagatgttagtgaccatagattacacagcatattctgtttctttccaattgcaaaatggtataatggtttgaccatgataataatgtcattgtatttggttacttttatcatatgatgatacagaattctgcttaaaatatttttcttgtgaaagacagacataaatgcagtgtaataattgaaaacattaaaatttatgttctacctaactgaaatccccaaatttcaaaaattgagatacgatgacttccatttatatcagggtgatgagtcattgttgccttaaggcttagctttcctatttaaatgtaaacttggtagatgtcatttggcaaggaggctggtagataattatgtttggaataacacatgcctttttcatctaattatcttcttgatgaattatcaaagagtatgggacatgcaaaacaaagcattcatgcaagtattagacacagttttataaaatggagatccaaaaacttgtgaaaagtaagacaagaagaaataaatttaagtaaatacttacttgcaagataatagccaatgtaaaaaactattctttgagaagaatggtaacattgttatatatttcaacagcagaagcttttttaggaaaaaaaaaaaaagactgcccctggtatcattgatttttgaccatataaaattaatttgtataaacgatttcaaaggaaaccaatgaggctatggatataatccagaaagagaaagaacagtaaacaaccctttattactctgagtagaaaaactacctggctgttattgaaatgctccaaatgactcatacttctattaaatgtttccttctcataaaatcagttttcagcatttttaagtaataagctattttctgaaaaacatataagctgagaacaaatcataaaggatgaatacatgatgtgaactttgccatttcagttaggtcatcaaaacaaagtctcacttgtctatatgattttgattgtcacattcttaagggcttctaatttatatatttacaaatgtgtatatatatatatacatttatatgtgtatatatatatatttacaatgtatatatataataaatacacatttttaaaatttttgtaaatttgtaaatatgtaaataaattgtatgtatattgataggaactacttaattgccctctatgggtggtaaaacaatttacacctttctaaatcaatgtagaagagtgtcagatttccttgctatgaattacaaacttctggaattttgccattctcattaagggcaaaggaatatctcacttcttttgtcatgaatgagattaaacatcttttcatatattttcgaactatttgcatgacatttctgtgaacactgttaatattctttgcccatttttgtggttgttgttgctggatgttagtcttgtccctttccattttgataagttatttagatttgggggagactaatattgttctataatataaattgtaaaagtttttccagtttctcctttgtcatttaagtttacacacattgatttttttctatgaagaaacttaatatttttgtgttgccaggttcatcaattatttttttatggtttatagatttttgtgtcagtgatagattattaaagatagtataaataattctgtcatgtttctttcataactttatttaaatttaaatcatttctccatttggaatttattttggtttaatgaatgaagcttggatctactttttctttttttccaaattgatatctcattcccaacaattcccggttcatttttccccactaattcctatgccataattttaacacaataaatgtccatgtgtatttgggttgatttctgtaccttttgctatcctattcttttactctctttttcgattcatgaataagtcattataatgtgttttaacctgttaaggctggttcctccctttccatagaatttttgtttcatagtgtcatttatttttccttccaaactttcaaaaaatcttgtgtaattctgaaaaatacaacattactggaattgctcttatttataaactaatataagaccagctgacaaccttatattaatcttcctctcaaaaacactgtatgtcattccatttttatcctcatggtttctttcatgttcttaaggatagttttatattttttttgttttgcctattcttactacattcactcctcagcagttgttttctgcaattataaacaaggtctttgtagtttctaacaggttattgtttatgtataatggagctattgattaacgtggattacccttgtaccttacaaaattattattttttatatttattaaggtaatattgacaaataaaattgtaatatatccaaagtgtacaacatgatgatttgatatacacaagcattgtgaaaggattcccctccacaaaaaataacacgtttgtcacctcacatatttacctttgtgtgtgtgtgagagcttaagttctactcttctatcaagttataattgtataacacagtattatcaacctgtcaccatgttatacattagaccttgagatcttattcatcttacaactgaatatttatacctttttaccaacttctacccgtttcgtttcatcctgcagcccctagaaaccatcatcctttctgtttctatgagtttctattttgtcaaatacctttttggtatcagtaaataaaatattctttttctacttgaacacatctaaaataagtgaatggatttaagaacattgaactatttctgtattcctggaaaaagtcccccttggtcatgatatattttcttaatatggtgctgatgggttctaattgacaaattgtattatttaacattgaaatgcatgagtttagatttctagagtgttttttctgtgtgattgttgaatattggtattcatggtttaaatagactgtggaaatatactctttttttcatatgttctgtaacattttagtaatactgggattataactttaaatatttatcctgatctatggcaagggatttctgataagagtacctatatctgttaaaacagtctgttgctttcttcataatttttctgcagctttctgagttgccataatacttccttctataattatttgacattcagttgtcaaattttcctataagatatttacctacttgcgtaaggaacacagttgtattagaggctagctgagtttcagctgccacaaaacaaaacattttctggaagaagctgcctagggctgctctgtatcttcttggtataccactgctacttcccctacccttctgcattttggaggtgaactaggtatagtaggatgaagatggggaatgtggttctatttccactcagtttcccagggcttctcccattatgagaagagcttttgacccttcactttgaggaattgtacttgtaccattttttcctgggatctgctgcatctgtagtttctccaaagggtctgttcacttcacttaccttcctcttgatggctcctataagaacccagtacttttatacttttggtgccttccttctagtttcattaacaatagaactttctatgtttgtttccttttcttgtgacttgtatatgatttccagaggaggcagggaaaatgatgagttatatagtcatgaatcaaaaaaatgaagactatctttaaagaaaagacttttaaaaacccatgcaaattggtattggtggcccatgactatgattgtgatcaatattctttcacaaataaaaataatctggcctcctacttacttgaaattcctgtcattgtgaagtctagtaaagaattgcttttctctttgttgtgctatgtattacatctaggaacacttacagccaaataggtgtacatttacgttgtgataaatgaatatgacttattttttttttaattttatttatttattcatgagagacagagagagagaggcagagacacaggcagagggagaagcaggctccatgcagggagcctgacatgggactcgatccagggtctccaggatcaggccctgggctgaagacgccactaaaccgctgagccacccgagctgcccaaatatgacttacttaaatactagattctctttaacagaaattgtgagtgttcaataactttgtaaagcaatccctcattagacaaaaatacttacaatattcataaaatatagaaatactattgtacatttggattataatgttttaccatgcctgatttacttataatgcttcaagaatacattattactatataatgagtgaagcagttatatcatatttattcaactgacaagtaatttaacaaatataaccatgaagtatgattttaaaatatcactcaaccaatgcaacaaattaaatggttttatatattcaacaatcttattctgagattatgcttatctatatgcatggaatttcatcaagattatgtggaagctttcccccccaaacttcctctttaaataataaaaatgtgattggatagttttttgatgacaatttttaaaaatattttatttatttatttatgagagatacagagaaagaggcagagacataggcagagagagaagcaggttccccatgggggagcctgatgcgggacttgatcccaggtcttgatcacaggatcatgatctgatccaaagacagatgctcaactactaagccacacacgtgcccctctgatgggaaaatttaaagatttttattttacttttcttccaaggcacaccgtaaatattctggaataaattttgatctggaaataacaagtaatggaagtattgctgatttatctttaaaaatttctgcaaataggggcccctgggtggctcagcaattgagcctctacctttgcagagggtatgatcctggggtcctgggatcgaacccccaacatcggggtccccacgaggagcctgcttctccctctgcctatgtctctgcctctctctctgtctctcatgaataaataaataaaatcttttaaaaaaatctgcatgtcatgtgcaaagccaaatttatagataatctgaaattagaactgcattttttttattttagaatgcacatttgctacgtattctcaaatatggcatttacattgaaccatattatatgttagggggaaactacataaaatgcatggaaatcatttagaagttattggcaatttagtgtagtcttcaaaagatcaggcttggcagccatactaccttgatttgagtctcatctttaccaattaggagctattgtgacttcaggcaaataattaaatctctgtaccttaatttccttatccattaaatactcccttccacaatttgttcaaaacaaactgctgggaggataaagtgagttaacatgtataaagtacttacaacaatgcctggcacagagtggtagctataggaggcttcctattgttgccagaccagattttgaataactgatatatcggaagcataacatagtcttcacagatatttaaaagcatgagatgaaagaaaatggatggatctaattaaagactgccaccacaaaggaatttttggaagaaaggaaaatgaatgctccagatatttaacacagaaggaaatcacttaacttgaagcatgagatttgtctgtgcttaaaagcctcccccaaatgatcttattcattttccatattattttatttgccaaaggtaacctatataccatattgaacgacattattatttaatgttgttagacataatagagaaggaaagtactcagaataatataaaagcctgcagtatgtgagaaatagctaatcataatatgttttcagtgtattatatcacagttaatgtttatagtagataagaaaagccaatgtatcactatgtcaagacatcctgatatcagatagaagtacatccaacctaggtgagcagagaatttcaattatattacaagaaacctaaatatttattagtcataattttcctcaaataatgtgtcaaatttaaggtgccaaacttccaaaaagatgtaaaagacctgtaaatcagcttgaaaatgaatgggggctcttggatggcttagtggttgagtgtctgcctttggctcaggtcgtgatcccagggtcctgggatcgagtcccacaccgggcacccagcagggagcctgcttctccctctgcctatgtctctgcctctctctctgtgtctctcatgcatgaataaataaataaaatatttttattaggaatttaaaaaattaagatatctttggaaaagaagtactgggaagaaagttacaagttatggtagtgagcctacaaagtagaagtcaagatcaacattatcttccccttaaggatgtgatggagttttattaaagtcacgcaaaacaattgttctctgtgcataaaacatagctaatcttagcctcaagatcactagtttaccaagaaacactgatgaaatttccctatccagaaaagagagactgtatttagtttgccactgagctttcagacttttatacacatagcatgcaagttgaagtacaagtactatataactaagggatgttattattactcccattgatagttttataaaataatcataattcttaagaaaccagcttaattgaaagcaatttcaattagaaccaagaaaaaacttgctgcttatcactgtgataaacatgaaaaagggattctatggggaggaataagtgctctttcatttttgggacccttaaaagcagaaagaaaactagcctcttgggttcttttaactatctattggcataagaaatgggagggtggaacaagatcctttgagtatcttaagagatctgagatcatatgctcatgaatgtgtcctgaagtgacatcctgctagcaaagatgcctacctatgccaatcttttgatacaatcactagaggagaaccagctctacccacatacatgatccaatgttttctttcattttcaaaatatattaccacttaatatttcttgaattcccaaagcagaaattcataatattggagaatggatactaaatttttatttgtgagctgaaaatgaatgttacttttgaatgttactgttttttttcacggcagtattagttgattttgctttatgcctcagaaacactttttaaactggggcatgtaagatgctgacacattaaatcatgtgtttccattactgtttattatcagtacaaaatgtcttctgatatgaaaataaaatgaagtaattttttaaaataaaaaatgtattcaataaataataggagatccaaaggtcttatcattaattacacataaaattcatctgtagcaaagtaatactttgtatatattacgcatttattcaagatttatatcttgttatattttgatttcaaaggacttgagacagcttgctaagaatcaaggaaatagtcaaacatcagagagtgtatctaaaagcatttattcacttattccttcattcactaattccttcaacaaatatttatttaatgtctaaaaatcagagattggagctcaaggatatgaagacgaataacatatggttggttcttcccttcaagaaactcaaaattctgttttacagaccataaacaaatcacttaaaatgctctaacagatacatgtatgaaagctaaggtatgggaaaggggcacaaataagtatatgtgtaaaatgctaagggacccctgaggtggatatttcattcaagttgtacatattataattgagcttaagttttagacaatacagacagattattttaatacatggaatagacctttttaaattacattcctgtcaactccctaaataaatcaaatggcccagattgctcactgttctctgacctccccctcctcctcatggaataacatcaatgaaaaaaatcactgactttctagggttagacaaaccaggattcaatttacatggttcaaactgagcctcagttaccttatttttaaaacataaaggataaattcacctctctgagttttttgatgacaagaaatataagtaaatgtatttgatattttgtaagcatttaataaattaaagttactattattattaagattttgtcatgttgtactctctgtactgaatgccctatcaccatccaccctgataaaattcattttgtctctaaagctctgcatttcatgtaaaacttaattttactcattcttctaatgtggttactcctctgctacatggttgggtttttatactgtatacactatagcatctctagtgaatatattttaagttgaattaagctcacctagcctagaaaaaacaaaagaaagacagaagagaaggagatgccaagaaaatgttaagtgatgtattaaaggtcacacaactatttaggggcagaagtggggctagagccagacttcctgatgctcagtctaaatgacactgtctcttagaaagcattgctatttattagcagaccaaatttattctcaactcttcacttgtccaatcctttaaggaattagtcaaatggataagtagtccaatgaattgtagcaagcacatttaccaatatgtattgagtaataaatgtgtgtatacatgcacttacttaatcctcataaaccctgtgaagaggccatgaatattatgtcctcacttataaatgagtaaactgagtcacagagagggtaaattccttcctcgaggtcatgctgctggttgagtggcagagccaggattcaaacccagcctccgtggtcttaactactcagtgtactgactcttgtttttcatatcatcttacattcacacttacatcttacactcatttaaaagtgtacaattacaaaaatatttcatgccagtaaagctaatctcttgtgaattattctatctcagtgacctgggggtcattgtacggttggtacctgttactttcccaagtaattcttggaactttctatgtgcaaagaagcctttctgttcactacaaatattaaaaacagagcagaaattattttctatcctttttgcccttactttaatttcagtctgatacaggtatgggggagtcattagacctcttttctaaatatttacagttctccttccagatgtatgatagtatgttttttgatttcttgttttttatgttgttgttgtggttgttagcctgcccttttgaagtcagacatggacgctaggctggattggtcaatgaaatgtgaggagtagtgatgcatatcacctgtaggagaaagcatgacaagccaatgcaaaacttttacattcctttcccattttggaggtgatcataaagacagggattaagataaagctgagtcccagaataaccttgcagaacagaggtccctgggcaactcataatgagcacatagactaaacaacaaacattccttaaattaattcctgagatatttatggctactaccaaaccagtttatcttgtctttttcgaggtttaattgtcattagaaatactttatatagtttatatagttatctaaggcagcttaattttctaatagttagcaaaatacatggctttgactcaacttttatgcttttgtgagttttcacaacatgcgttttctatataagtaaatgattgaaatatttcctaggtattaagacaggttttaaaggtctaatcagcactgaaatagcttatgctttcacttctggggatgttccctgagataggaagttattcataattataaatatgctagtgttgtaaatgttcagtttttgtttttactgttgattttgagttgctaggaatgtttagttcaccctccttccatatttgcagactcctggatgtagatacaccaatgaaagtttcagattgttttaatacagtttggttaagttttcaactacaacatttccttcccacaattcccctatgtgcatgcagcacatagtatgtgtgtgtgttcatcacagcctccaagaactgattttgacaaatctttagagatcattccagcttggatcattctggagtttcatttttctaatggtcaagcccctgattattcaaactcatggaaggaaatagtatatacataaattcaaaaccagaactgatccaaaggcattaatatttggctctggagatcattatataacattttatgaaagcagcatctttgtcaccctagttttgtagcttctggctttccctggacgtatattaagtgaacatggaggcagaacccagcagtgtcctggaggcgagaaaggccatcttcccttacatttaccaccttattttatgtccagttgatatgtttattaagtagatatctgattccttgtctctgttctgtatacatctatccaaaaactgaatatgccacaaaagtcagccttcctagttttgcaaagcatcctatgtgccagaaaaaaagccatttttaattcagattttgctcttaggttttattgtatttctgtcagatttttcctgagtcatgcaactttttaaatgttcagctctcttcgtgtcttcaaaccatgatgttttacaccaagtccacaatagtattttttaaacagatgggtagttgaatgaattagtatttaataaacatcatagtaagtattacagtagaagattatgtgaacaccttaaagtagtgatcatcatctctggtgacacaattgaattatgtggaacattttgattcctgagcctcatttccagagattctaatcaagttggtctaaagaggtccctggaaaactagtattttaaaaagccctttagataattctctgtgcaaccaaggttaggagacattggctagagtcagagagaattgtacctcattcttcacacttctaaagtgttttttgaggtgttttggatcatcacacttctaatatggagaattttcattctatttcttgtgtgatacaggtactcattttgctcttgtctttgtgcttaacttttccaaaggcattaaagcagttctgagaaaatcactgcagggggctcctaaaattacaaaataaatttctacttattaagattactctctaaaacaatatttgccagttcccctccgccagagacctccagaaacaaatttgtgaacaacaaagttaggtttcttacttcctgcaacaaggaaccccaagtgtccaaggacactacacctcctgggtaatggtgaagaggatgtctcagaggggataacataggaagaagatactcataggtttggggacaatagtggcaggtattggtcagaatattaaaattagggagcttctggaacagtcagtggtcttatttttggaacagaaaagtttgtgaggagtctccgttagatcaatttgccaagtctaatcttgggacataaacatttacatgaactgatgctaaaagagtgagtttcagtagttgatgatggatgtgacagttctgatgtggtacagtttatcctgtttgaagttgtgagttttagtgctcagctttccatcacaacatagctggtatgcagcagagcttttttacattataagatgaggggaggggtgttttccatcatcaatagagtaatgcttagctatctatggccatacatcaatatattatatatattccagtttaaaagaaaactataggagctattcaaaatctgtttaataaggtttttactcctgctcattgataccaaacaaaacattgttcttgtttgtttgctcaaaaacacaaaggtaagtcaccttattacttctaacccaagggcttgatacaaatcaactctagaggccatcgggggttgggcattgcagtcttcatgaaaatcgcagaagaatgtcaaagaagctgcaactcattagaaatcttgtcctcctaatttatctcatattgcaaagaaatggcatagatttaaaagaaaaaaagtagtattttcaagtctgatgtttttgtttgtggaaattctgtatgggcaatagactatctccctacacttaaaattataggctgaatttaggaataatttcaaataaatggttagtaactatttcttactcatgatgacttttctataaaattataaacctagtgttcagatacagcaacattgtatacaaatttgagtttcaagaatgctgcaattgcattttccagtgttcttagcagcatggctttggaataaaagtcttgaggtagatccgtgaatgggacaccattcaccactgtgtcatctagggcacattgaattgcctgtggttttaaatttgaggcttttatttttaaaaatttctatttgaattcacttcagatgtataatatgttataaaaatagtacaaagaaatacccagatcttcacccagcttcccaaaatattaacatttcacataacaacattgtagttttcaaaaccagaggagtactattgatactatactattaattaatctaaagaacttattctatgatgcatggatggctcagaggctgagcatctgccttcagctcaggtcgtgatccaagggtactgggatccagttccacatcaggttccccacagggagcctgcttctccctctgcctatgtctctgcctctctatctctctttctgtgtgtgtctctcatgaataaataaataagatcttttttaaaaataaagaacttattccattcttgtcaattgttactgatatactttttctgggccaggatcccatctggaatcaagagtatatttaattgtcatatcccttgagactcttttaatcttagacagttttttctttttttttcatgactaacacttttgaaaagtacagttattttgtaaaaaacttctgtccatttgggtctgtctgggtgttttctcatgattaaatttaggttatttattttttataagaatgccaaagcagtgacattgtgcctttttcagggcatcccatcaagaaacatgttgcttattctcattagtgtggatactaactttgatcagttgaacaaagtgatgtctgccagatttctccactgtaaagttgctattctttcaacaaacaaatatttttttaaataactatatgtgctctccctccctcctttcttctcttcctaaatatgatgtgaagtgatgatagtggaaaaatgtgagagccactaattaagctggtggattcctggtattattccccctgcctcttctatatattttaaaatagagatttcttatgtgtaaaagacttaattttatagtaaagtggccttctacaagtcctgggtagtttagccattagtacgttcttaaaaatttggtcttaaatatacctcaacccagcgttcctgggtggctcagtgggttaagtgtctgcctttgactcaggtcatgatcatggagtccttggatggagccccacttggagctccctgcttagcagggagtctgcttctccctctccctctgctttcctcccctctgtttatgctttctccctctctctcaaatatataagatctctaaaaatataaataaacaaagaaacctcagccccagcagcagccatatggtagagtgaaagaaaaaataaactgcactctgctccattctagaacttaaagctttcaaaaaactggaattatatgtctttctagctatttccttaacaacaacaaaaaataataataattaaagaaaattgcatgctgtgcagcatagaaatccattataaattccctatatttacttaaattatccttccccctggttttctttttctgctaaagccacacatgcttggtctcaactcattctataatttgtgaataatttaaagaaagtaccttgaaagcagaatcacttagaatccaatatacatttggcagtgaagatgaaggatccagactttctccttggataacttgactatgactgagctcaatccaaggttcacagaggccagagcagtgatctcacgcctctcagaaaatgggcaattaatggtacttgggctgactgcaaccagaatacagcctgacaagagaaaaaactacatgctttgcagctgtgagcatgatgtcgaagaatgcctacagggaccagaatgggctggaacactctcttgcagttagcatcatgggccagaagcaggagatggataacagagtcacatactgtcagatagcacatgagatgctgtgcctggagctccctctgcctaattaataaggagaaaatacagtcaatagattttgagcacagtaactgaaatatgcctgccttctccctataaataattgtatttaagtgagttatgtcttaatgtattctttgagcttaaaaagtgctaaaactcaagttttaatttaaatatcttaaaacaataataaattatattcatttcatgtttaatattttacatggtgctttcatccttaatattttgcttgacccttctaacacccatgtgatttaagtccttcctccatttcaaagttgaagaagctgggccatggagagaatacaggtgacttacagcagatgacataatcaaaatataattggtatcaggaaaaacaaaaattatcaggattttgctcatatattttatcaatgaaggttttctataactcagtggagattggaaatactaaattctgcaggccagttcatcttagcaaatctcaggattttgcaattttcctttgagaactggtcaatctgtgcaagggtatcttattttaattacatggggcaagatgactttaggctgtgagttgaggtgagaaagaagcaaaaaccataggtaggctaaggtcagcttctgagagcccatgaataggatacaagaagctgccatggaaaacaaaaatagggtaagagtatgtgtggttggtagagcactagattaattcagaagtaggtgtcaaatctaatttgtataagcatttcaagttgagtaagacaaataatctaagtttgattgataggccaactttggaaactgaggcatgtactcagcctatccagttgacttcactggaacataatctgtttgagaagagtttcatctctaggcttatccctagaagtctgcctccatgaatagtaaaggcatggcccacagaaattggcaaagataggcaaatgattaaattggcactaactagcttagtgcttgagtaagaacaaggaaaaacaaggaaatctgactttgagggtgcagagaaaagtaagtaaagtgattggccatggtgtccggatcatttgatgattgcaatgtaaggcatcagatccatcatctaataaaatagatgaagaatagagaaattgaaggattcaatgtttaaatgagattaaaatgcaggcaagacagaaacaaaatgttcaaaactatgaagacagtaaaaatagcaggttgccagggctcaggagggagggaggaatgaatgggcagagcacagaggatttgtagggcagtgaaactatttcataggatacataacaatggatacatgtcatcatgcatttgtccaaacccatagaatgtacaaccccaagtacatgatttaatgtaaatcatggacttagagtgataatgatgtgtcagtataggttcatcagttctaacaaaggtactactctggttgggaatattgataattggaacagactatacatatatggggacaaggagttatatgggaaatcttacaccttcttgatattgctgtaaccctacaactgctctaaaaaacaaagtctattatttattcatttatttgtttgtttgtttatttcacaaagtctattttttaaaaagaattgagagggtagatggatatggcttaaaatgggacttcagactttaagatgaagaataagaatggaaaatataggcagagatgggcataatagatgtggttaaccaaattattatcagaatttgagtataatgtggttattgattgagcagcccatccccacccattcctgtccacagaatgattgttttaaatggcaaaaccagcagagcatccaaaaacatgacaccactatctcctttactgggattctatctatatgattatctgattacacatccttgggaaagcttccagaagcagatccatatactcttcagtccctaactataataattaataatcatattagtgaaaaagcaaaatgtttgtctatgactttagaggattttggcatataatcaaagagcatcctaggcattgcttggaattataagagaaattcaattattttgcttattcagttcattaaaacaacaacaacaacaagcaaagcaacaaataaaagaggtattccttccttggctctgagaattagcatgaatgatttaacaattagaaattattctcataaagaatttttaacaatatcttaaatgcttcaaaatatattgcattctgtggatactctgcaagatctaataattatgtagactttccctgtgagttctgagacacttcccttaattttcactatgtttatgttaacacttactgctatggtattttcacagaacaagcattgtcatttgaacttcccataaaaagcactgtcatttgcaaattgttttgacatgtaaaagatggaacacattttccatccaaatctttaggagaatcacaagatctacagaaacagtatgcatagtgattaagcaaagaatacctatatcgaaaagtcccatgtttgaatcccagcttctctacttacatagtatggtaacacctctgttgggttttacaaaataaaccacagaatgcaatatatttcaagatattgaaaaatcattgaaatacaaattctttatgcaagaaaatttaggcaagatgtttaatctatctcccaccaccaccaccaccaccactgctgccaccattttctcatttgtaaaaccgaggtgataatggtttttaactcataagattcttctgaggataatgtaaatcatgtaaagtgttttgaacagcgcttggcaggcaagtaagtgttcaataaaattgacaaaaagtgctttcttaatgaatgggcagaacacagaggatttgtagggcagtgaatggcttctctacattgaatacttagtacaagataccatgtcgggattttttatatattagtctgttaatctttattatagccttcagataaacaatattatccccattttgtatatgaggaaatgaaggcttgggatggttatgatactggccaaaacataccagtggtgagtgagctgggatttaatcctgctctacaagctctagagcacacctccttagatccttcactatcccagcttccttaggtttgtgcctgtgagaaaattctcatttgcctcttggaagaaaacaaatctaaagtgattatagcaatttttgcaactgtggtcactcacaattgaaaatagtaatgacttccaagaccagatatcaggcatgcccgtacacagtgtcccaaatgagctctttatatcctagatgggtaaattgtttattgttgtttcgtcttttttttttttttacttttgcacatctgtatatctaattgtctgagggacagagacagacagagacactgaaaatgccttctactaaatcccaccagagggggatcacattgttgacacacttgtcttcaaaaatgagcccaggaaatggctttattggcccatgggcaagtgaacattggaactctaatttatacaagtgctgccacattataaggcttcatgaaaccttccaagcattttacatattctttacacagaccagttccatgtacatctagaagaaacaaaacaaacaaacaccctagtagggcttttcaaaaatgaacaaagtctatgcattggagtatgtgtttctagtcaggtcattgctttcttcttagcagactcagtcttccaaggcttaatagagagtagattgccataaaattctcaccaatatctttgagtgtgcaaaattctttctaaactctgaaagcaaatgaatgaggcctttttgcactgcttcttggttcacctaagtgtgtatgatatatgtacaatggaagtaacccattatatcaaaataggttgttcaaaagtatagaacaacccatatttttgaaatgcttcatttgtaaagatgaaaaattggagtcttagggatgtttaagggcttgttaaaaattatatatttactaggtgatccagtaaggaattcaactcaggtgttattccaatcacatgcttttcttactgaaccatgttgcttttttagttcagtggtaaaaacaaacaacaacaatgacaaaacaccacaaggaataggaaagtttttggaatcatgtattggtgatgaaccatgtattgtggatgatcacgtgcaatggtctctttcaaaacaaaccaactataagtaaaaaaggaggactacaaaataatgtagatatttgcaggacacctgggtagctcagtgttgagcatctgccatcatctcagggcatgatcccaaaatcctgggatcaagtcccatatcaggcttcccgcgaggggcctgcttctccttctgcctatgtctctgactctctctcagtgtctgtcatgaataaataaataaaatctttaaaataataataataataataatgtagatatttgttaggaaatcctccctccacctaaatcacaaattgaggacatacaaaatatggacacaagtgtttcttctcattcaaccaagaatatcttaaattccatgactttaaatttaaaatgtagccattattttatcattctttttatagaccttgtgtttaaagacttaatatgatgggcataaatatttgcaaaaactgtattagaatagagcatagcagcattattgaagatttggttttccttctacacatttctttagaagatggctcattctgtaccatccacctccccacctgacctctggtaaaacatttaaattgcaaatagataagagcaagatgatacatttcccaaagttcaaaacatatgcataatctgctggacatcagaaagttatggtcgtataggaggaaaaaaaagaaaatagtctgggtgatataaatagatatcagtgattgttctctagttcataagactacagacttacattgtcttccttttgattccatgtattttctactttttttaatgagcctatattacttttggaatgagaaaaggtgttttgaaagactaaatgtgtttgtgattaaaatcatttagagaaaataagttatggacataaatgcctattactgattaaagcatgtgttttcattttatgacaaaacgtaatcctacaatttctccccaagtacttggagatctaaaaaatggttttttaacattttgtatgatggttgaccaactgtcctgctttgctcagaaatgaggagttctctgggatttaggattttcattgctaaaatctgaacagtctcagattgtttttactatatagtcctccaacatctgtatggaggtgttgatccatagtcccctcaagaagtgccgtttatccccagagagctcatctggaccacttacagaggcacaatgagttctcgtgaagtctacagattttccatctgtttagatcacatttttatgaaatctgttacatgtgaatccagtcattcattctgtaaaggacctgcccaaataagtgtctataaatatgtgtctacatgttctggatatgaaagaatgtgaaagtaactatttaacaggacagataggagccctaaagcaattgggaaaagttttatgtagtcttctaatactttctggttagtgtttcaccagcagtagcaatcatggtttcttgcagttaatttactttattctctcttgaattattagtctagcaaatcctggtaagacccttccagatttttgattctagaactctctatgacagaagacaatagaaagaaaagagtacagctgagggaataagtatggtgaattggggcatgaggctctagagattttcagggcctgacgtttttaaatgcattgccaaagctgatccagcatatttccttccccgcattaataatcacttggcactaaagaaacacttgctgatagaacattttatggtatggtgttttattatctctgtcttggaaaaatggtagccaccgataatctaatagagaagagaaattcacttcattaatttattatagcccagaaccctaggcagggtgtttgattacagggaatattctttatttctgccaaattaaaaagctggcacaacccagtagatgattaaagcttttggttaatgccttctggattctgttttctaatagacccaccaagaacttgtaggttaaacttaagcttttcctaccattttgatatttgcttggtttccccaatatgtatgtactttaataacaaactcacactggctatattaggtagttatggttttaccaacacagtgtcatagaggcagagacttgaaccctgattctggcagtgctactgatcactcagtgcatgacttcgtgcgctgcacttaatcactataggccctggtttcttcatctgtgagatggaaaggttagattaagatctctactgctttactatgttatggttcatgtttggattaaaagttggggataaataagtatgaaagaaaatttctttcaaaagagaatacatttaagttctgttaaatgccagttaaagattgagttatttgattttagtttgccacgtaatagcctaggggctattgtgatgtatgcttcggaatgattattgcagtttatgtactatgcctgacagaaggaaacatctgggggatttctgtgaaagctgagattcactcaataaaagacccttttgcttttggacgagttgcacattagcccacctgctatactttgtactttgtgagagtttcaccatctgttagcaattgtttccttaattaattggtagaatctatgatatttttaaatgcttcatgtcaattttaaaaaagcaaaaagatgcgagttacatactgttgtgcacagtgcatgaaaatggaataaaaaaacccagcatgttagagctgagtgaggccttagagatcaattagtccaacttcctcattttgtagatgagaaacacatccagctcattgttggcaaagcagagaacgtgtatctcctgatcttttttttttcattatatcatcagcaaatcacatctttctcattcccagtaatacgtttcttctgatttttaaattcctttggttttacaataagaggtaaaatagaagaaacaaactttttaaaactatttctttgaatgaaagttttctgtggacacagttaaaaagccaaatagatctatagattttgtaaccaaaaactggatgtagtctattctactcattccatgtgcagttgtgatacccaagagacaccacattcatttctttgggatgttcttttttatatttactttcacatttctaaataaatattttatactattacctcttgataattacttttaagtactatctactgatttactactatagaatgtaagactgattcttctcatgcacacctccatccaaacataactttccttccacttcaccattcccatcttctaaatatagttataacattcatttcggttagattatactcagtgttggcatcttataattatttaaataacatttacagctgagccattcagaggcaatggttacatttcctatcttattcaactttttctccctgtttttatcaccattgttgacttgctcacttaacctgcatcactatcagtaattcacacccaagccctctgatacaagggtaaatttcaacttagtacattcagaatcatcagatgtccatcaactttaccttttcctgagcctttctgttgtaatatatactgatagccttctgggcctactagcataacccagcttcactatcctttggttactcatttagttctctcatatttgaattctctttccttatttcatttacttctgtattttaagagaaagggtgaagagaacataaaatatttcataatcatagggctgaaaatattatattctgagctcactcttgcttaataatttggctggatataaaaatctagtttagtatagaaatcactttctccttgaaattttgaagcattgccgcatagcttccagtgttattccagatttcattgtgatatgccgtgacttggatctttattcatcattttcactgaacactcagtgagacactctcaatatcaaaactcttgacctccagtttgtagatattttgataaattattttttgtaaagttctctagttcattaaccagaacctaatctcacagtgctggtagtcaagtatttttttcaagcctcttcatgtatacatctgcttataccctaatttcctatggatcctctaacaacttactacagtcaacaatacagatagtttaaaacaatgcagatctattttcttgtagttctagagttcaaaagtttgaaataaatttcactggattaaagtcaagattatggcatggatgggttttttagtaggctctagagaagaatatatttccttgccttttctagctttttgaggatatctccactcttgcatttcctgcctcatggttcctttttgaatctttaaggcagcagtgtagcttcttcaaatctctctctgcttccactttcatatcattttccctcctatgaggacttttgtgattatatcagatttacccagataatctaagatgatctgcccatctcaggatccttaatttaattacatctgtaaagtgcttttcattataaaaggtaacatattcataaattctagggattaggatatggagatcattgggaagccagtaatcagcttcttacacgcaccattggctatcacaaatcacatccctaatctcagtatcaactggtgctaaaataggcatcacctctatggtgagagaaactgcagaataaaatggcaaagagcatcaatacagcatggagtgaaggaatgaggcctccagtataatcaattaatggcatacatggatcatgtatacatttatttatatctttcataatatatttaaataaagatttataattttcaccataaagctcttgcaaatttttgttagatttattcctaaatactcagtagttaatagtatttaaattatatatttaaattttttcattttctaattatttatggcatatagaaatgcagttggtttttataccaattttatattcagtcattttctgagcattcctatcaatctcagtggtttgtttatttatttacttatttatttgccttattttgttatcagcagaacaatcttgaatagagtagtaataggggtgcctggatggttcagttggttaagcacccagctcttgatttcagcttaggttgtgatctcatgagtcatgagattgagccctacatcaggctctgcactcattgcagaatctgcctgggatactttgcctctccctttccctctgcccctccctcagctcatgtgcacatatgtggtatctctctttttctctcaaataaataaatctttaaaaaaaatcagtgatagcaagaagacttcttttgtgccattgcatttcaatattaattatgatgtttgttatagggtttttgtactgtagatctctttaatcagttaataaccagttcttcctctttctgatttgtttagcaataatgtttcttttataattaaattcatttaagatatagaaacaaaagtttacattttgataaataaattcacctgatcataaaaagccctttctacaaaatatctaggttcttgatatcttacttccacatgcatcttgaaatttatataatttgtcttgaatcacaatcttcccagaattatatgagtcactcattaaatattctccctagttaaagtgagcttcaaaattgtttggttaactgtgtctaccattatcaataaaaaataacaacttctagtaatatccttactcaatttaccaactgcaaagaactgcaccgtgaaagaatcttggaattctttggttcccatttagaatactcacagcaatggcctatcaagactgaagtttcctgagctaattaagctaacaaaaaatactgtgaatatattgtagtccagaattagaaagaaacaaattcttaattcaaatgaactctgcctcattcagaaaggatgtatgttgtttaagcatggatgtttcaacaatttttaaattctctggatatatatttactcttcctaagtacttcttccttatgagagtttagatttattcatttatttatttatcattcagtcatttagggactattttttgagcatataccatgtattagggactgtgtagcactgggatacaacaataaacataattttatctgtactttaagcagtttacagttcagtagatgaaacaaacaaatatataagcaattaccatatagaacacatgctactgtagaatgaatgtagaatactataggatcctgaaggagattctgtgtcttagaatactgagaatattcagtatcttagaatagtcttggaaaagaagaaaaagattttttaattttcctgggacacctaaagtataaataggagtcacctagacaaaaaattggaaagccatttatacagagggaatagactgtgcaaagacgcagaagtgaaagaaggaatagtattttcaggaaaattattcatgtaatctgaggcttaaacaaatagaaatagagataagtctagagagtcaaaatcatgaatgaccctataaatttagtaatcatatgtatcacccaaacctgtatgttttggaaagtgaaagagataatactaatcactatgccataaataaataaaccagatttccaacaaattggtataatctgagacaagctaagatatatggccttacttataagcaatgtaaaattctcctctttatcatggggatattgaggaacctttgatgggtcttaagtaagagggtaggaagatgacatatttcgatattcatctgacaaagattgcttttgctgtagtatggactagtaatgggttagggaagcagaggtctgcaaaaaagaagagaaatgggaaaaaaggaaacatacatgtgttgcaataattcaagtaagcaatggggttgcctctggactagcaatgcagtagtaatggagatggtaaaaagttgatgaattttatagatatttaggaaataaaatcaaaaggacttgctgattgagtgtttgaaggattcagtagtgaaagaggaagttataaacaccaagtacagtattcatgtttctgccctgtgcaaattagttgagctgaaggatccatcacacaaggtatcagccactgaagatgttgcactgtgttttgtgaatgaatttttgtggacgagtgtagttgggtttttttgttgttgttctaaagtgttagactaagtcctaaatgatgccactgaatgaatacagttgatgctataatagaaacacggagaccagttggtggaatacaaagcccatgaagaaacgaaagcaaaggcatgcagaagcaaaggaatgtgtgatcacagactggtttctagaatatcaaaggagcaacaataaatctttgtgaataaaggcaacctttccattcctggcatgggcaaatgagaggtcacataatctgagtaaaatcttgaacaatgtgtagtgaagtccctttagaaaacctgaaatcttttcttctctcatcttcttttctcttaaagggttttgattttatagaggcacaagatgagagagccgacttctagataccataggaacaagaaaaaattaatgatcacaatgtggatggcagtcaattatcaatgattgttataagcctccagtaatgaggggcagaacataaacagagggaattttggccagggaggctcagaaaccagtccctgggagggtaaaatagaatccagcccagatgaaaattgggcaatattcatcatgatgtcagactaagtaacacggtgccacaggctgaatccaacacaaagccctggaaacaaaatttagaagctcaatgatattatctgaacaatcaatacaggacaccaaaaccaagagaggagtcccctactccattcccttattcgttccacaatctgtctctctgaagccctactcatcagagcaggaacacagacagcatttaggctgcttgtccaccaacttcaccattttttttctactttacaccttaaaaaggtactgtgggcagtgtcagcagtgtccagcctttccagagttctctgtcattcatttcctttagcattcctgctccttccctttgtataatcatatcatgctctctttgttcacttgcctgttttcagcactttacatctttcacacacataagcttacttgatatgccatataatgattaagagacctgactctgctagctgaaagactatttttaaatccctttctgatacttaactttcaatcttggacaaattacttaatctttcagtaacgtggtttcttcctctgtgaaaagggaggtaacctaagaaattgaatagttttaagagttaaaatgataagatatgaaaagcacatggcacattgactaccacataatatgcactcaaaaatattagcaattcttattaccattgttaaccttgaacctttatgcagtagacagcattaatacaaatatattttttataatataaataacatacacacacatactccaaagagactcttaaaggttaaataacagattcaaagttatacatctccaatttacagagcttcatcacacaactttagacctcaggctcagtgccttacttggtaaacctggacacagcactcaagcaattccaggaaaaatgttaagaccttctgtcaagattcaatctgacagaacaaaccaaggatgtaactccattaaaatggccatgttattgcttttagtagctgtagtgggaagcacagtggtcccttcctcaagtcacctaagttgttttatttgccatctaatccttaaaattggaaggaacacactcagcagagctttaactgaaacaagcatgcatatgtatgtacctgtactgtcctgtctatatattgttgtgtaataacccccccccaaacagtagattaagaccataaggatgactgatttgcttataaatgtgcaatttgagcagggctccatgggggaaattcatctttgctccaatgacttcagttgggacagttgaaattagggttggagtatacactttcaaaactggatcattcacatgtctggcaattggagcactgggctgagagttcagctggacctgggagcctctgtttctctccatgtagatgaagctctccatgattcctggactaccttatagcttggtagctaggttttaggagtaaggatcatgagagagttgtatcactttcatgaccaactttagaagtccatatagaggcatttctgctctactcttcagtcaagccaattacaaagactcactcctttcaaggaaagagaacattcacatctactcttagaagtagcaaggttctggaagagcatgcagagtaggaaatataggaggtgggaaatatatacatgcatatacacacatatgaaaaatatatatgtacatatttcatattcaaatgtacatattcatagccctatatatatgaataagctatgaaaaatatcagtctcactctgacactacctatccctttatttttttcataataattgtaatcacctggcatatcatatattatttattatctgactgccttctactagaatgaagtgccatagaggcagaaactttatcctattttattgctaaatcattattacccagaacactgtctgacacatagtaggtgctcaatttatttgttgactaaatgaattcactcatgcataagtgaatactgtccccaatatatagggccataaattcaccaacatacatgctggccctcaggccagactgtgaaacatcagttaaaagaaaggttagtggcactgtcttccatatacaaacttggtacatgacaaagaaagaatgttattgaagctcctgatgtctgcatttgcaactccatggaaattataactctaaaacttctttttcctctgcattttatttctttcatagaccagccattgctaaaagcctcaggagagggattttaagatggtaactgtgaagtatgaaaaaggcagtgacagatataaggattttatttgccttccacaagatgcctcataaaggcgtagttaataagttgcctactttgccttaaggtgttttctgtagctcataatagtatttatttcagaaagagtatcttctgtcacattaagggtcaagtaaaaatgatgtgaaatgaccttgtttggctttagaaaaattaaagccctgaaagcaaagaaagcaataaaagattattatgtgccttttgtcagatatgagatacattataatgaatcccttatgacaagtaccttcacagctgtagttgtaaacacttatatcagctgctaattgtgatatctaataggatccttgacactataatgtggcctcctccatagacattttgttgtaatgaaaaacactaatagctacagtgaggaaaaagcaaatagtaaattcatgaattacataatgtttctctggtaaagcatttttaagataggaaattttgataaaagacaaaaagagtaatataaaattctaaaatgtgatattgaacacgatttggagaaaaccaaacatctctttactcaaaatctctaaagatgccagtaagaatatttgtattaaagcctccaaaagaagcttttatatgagaactacaacatattatagcaacataatttatggattctctttaatgttttcccttaatatcaacatcaatatgtgatcacatatagctatatgatatatgaccaacacatatttataaaataaatgaatacaaggggggtgctatgtgttaaccatcaacagaacatgatctttcctatcattttcaagaagtgaatatggatttgaatctttacttttaaatattctgtgaactggcagataaataagaaagacatgaatcttgcccttgaaaactttattcttcatacatggttctaataagtatgacatggcattctaatggaaatggtgttggggaacagctaagaatagatagatctatttgacacaatattagatttttctactgaaggaaaataaacttttgtttgaaacatcaataaaggagttcacttattaccatcatcattattattatcatattcatgatagataacatttatttagaactttttaatgtgctggaacacactaacactaacagctttatgtattgtgtttattaatgcatttactcatcacaaaatctctctgaggcaggtactactactagccagttttacagatgaacaaactgaaggacagacaagttaaatgtttggtcaatgtcacagagctaatagaggcatagtcaagatttgaacctatcaactcaactcctgagtttatgctcttaaatatgctgctttgttgcttcaagaaggcacgacattgcctataaagaaaaatttaaagtccaaattcaggaagttatatgatagtgaaaatctggaatgccattggatattggggtataggccctaaaatacagtttcagaacttcacacatgctatcacacttcacccgcacacgaaggggcctttcccccactgcattgtagtaatagtacttaatttatgccaagaattagctcatctaactcttgaaacaactcaaagaaataaatacttgatttacaaatgaaaactcgtactcagaaaaattgagtaacttgttcacagtcaatagctaggaaccacttagaattttaagtcatcttaccacaaatctgtgcttataatcactatctatgtgtttctcctcctgaatgcacagtagagtccttagaagacccattaataaatgtaaatggttgggtgtcacttctagagattctaatttaattggtcttagttagtggggcctaggaataagtagtttataaaagttccccgtgtgaatttaatgtgcatccaaagccaagatcactactctatctgattttgttcctcatcttaaaagttatctttggcattagctccctaaaactcagtagtctacattatgctttccaggatagggtagggtatgacaggaaacttaagtgattggcaatctccagtgctttcttatatgggttttattatataaaacgctgacaggtttgaaatggatttctttatccagtaagaaataactaggtgattctgagtaggatgttataattttaagtttaattttttcctgatctttctgattatctttccaaaacatatgatctatttaaattgccttcttgctgtatttctatcttagtatacctggcccacaatttttagctagtgaagctctgattcacaaaaggataacataagggtcacctgattcatgctgcattatatttggcaaaagaaaatgtctcaccaaactatagttgaaaatggcaactcactatataaaaacctatctttctgcagtttgtgtttatttctttttcttttttttttaatatttatttttcaaagtgaaaaaaaagagaacattgagctaatgggcaagtctgaaaaagaataagttaacttaagggtgaagaaatatagtccctgagatacaatacttgatgtctggattatcacgtgttaaatattggataaactagaaatgcatatctggacagataatttttcagataaaaataattagaagaaacaagcacaaatccaacacctctcttttagcaaatgacctgtctataggctctgagctggaaatgtggatagcaattacagaggctgttaaacagaagtgggtattgtgttccagagacaaatcagtgctgaaaggttaagtgctttggcctgtctttgcaatgttaactaaagattcaggaaataagaaaacaagcatggatataaaatttgctgtggtaatgggggccagggaaataaggtaatgtgaaaccacaatttaaaattaattttaacctgcttgtcacaaaacttaaacaaatgtaactctaatgtgtggaattttacttttacttctgctattacagctctcaaaagacaaaggctacctgacaaaaatgtttttcctctgagaagggtgacactttctaaaaaaaaaagtcaattgccattaaataaaaagatgataaaatacagacttttctatcatcaggaatattcagagtcatcttttaatgactttgaaggcatagtggccatctttcatcctgaactgtatcacgagattttaattgattactagattttgatggattaattccacaattttcctttattcacattctctaaaaagtacattaatcaaagatggaagatatgaattacaaattcatttctaccaccaacataaatttttaaacctctatcgtcatttattagaatggaactttgatctatagtccgatacaattttttcattgagcaaaaactttcatttcctaatatctattattttcactagtctctactaatttcatctatacatggtacagattgttttgacagacaatgccatcttatagtgtaaggagcactggacttggatccacataccccaaagccctgggttatccgggattcgaatctctgctttcaataccaaaagtcatgtataagccaccatctctacatttatcttgttagacaaaaaatcctgtgtgatagtcctgatagaaggatggtggtatatgggaaactgctttgaaaattattaagtgctttataaatgtagagaactggaatcaggacctttctaaatcatggaatctgcatctctcattatttgacagcaagacaaaagggagtacctgtagccacatatcgcatatatctcagagaagaattgtaggtaagaccctcattaggagaaactcacttcatctaatgaccataaacaaatcattaaaaaactcctctgcctctgttatcccattctcgatatgagtataaaaacaggtaattgtatatattgcttgaagtaagtttagaagaaataagagccacacacactttagttctgtgctaccaaggagacacaccattaagttatcaaatgatattactgtgaaacccatatgttcttacttgtaacattttcatcttacaacagttttttccctgaaaaattgggcctaaagcaagattataacatatgtccatttatcctgttttttatttctcagaacacaagaaacatttataagagatttttcctgaatttgtaccaagccaaatgtaaaaaagtagctgtcttctgcttctcacaaaatacctagctcatctgcaagcagtggcagcagaggaaaatgaacagggaagtctggagtcatctttgctgggtgtcacctgttttctctggcctctggcccctgttcccccattagctctcctgcattggcaactgtacagctgtgatatggttttatgatgtctggcttgtgctttcaaatgctattattgctgttttgcccatctcttctttggggactaagagtgcaccttccattgcctcctaactcagataatagctgagacagtactactctcagcctgtgttctacaggctacactgatgaacagtgtagcctgtagaacacacctgacctgaaggctagaagtacagagcagacgggaagcactgcacaactaggtactccatagacgcatttccatttcccctaacctttggtaaagctggacagtccattctttcccagacacagcccctgcttttccacgtccacattttattattttgctttgactgcccaggatgcccagctcccacctctatcccggcttttaaataccttctcatgttttgaagctgtgtcaaatgctagccatcaccctgagacttgagggtttcttgcaaatttttgtcagctctcctgcgaatctctacagctcttgattcatagtacccttgggtaaatttgcacattcttctttgtatatgctattttacttttttcctaatggtttgggccccttagtaagagacattagctgtgtaaactgggacaagtgatttaagtccactgtgcctatttttttcactcattaaatgaggattacaaaagcaaaattgttttaaggattaaataagttaatataagtacagaatttagacctgtgtctgctacatagtaatattaagtaagttattcgttttgtaataacattcttatacctgtcatatatattataaacatatatgtggatgtctgtgttttttcttccagagatttatgctttctgatcattccatttcctaatatatttttttactgtttattaggttatgagtgaatgtttttaaataagtaaataaaagaaagtaaagctcctttggacacctgagtgcctgggtggctcagtggttgagcatctgcctatggctcaggtcgtgatcccagggttctgggatccagtcccacattaggctccctacagggagtctgcttctccctctgcctatgtctctgcctctctctctctctcggtgtctctcatgtataaataaataaaatcttaaaaaaaaaaaaactcctttgctattgtaattctttagagattgaacttttcctaaactgaattgatactgctttagtttgatgagcgactcaacttccaagtaaggcacatttctttgtaaaacttcaaaatgtcctcaaacatagggcgtaatatttaattttttattcattcacttaactcagctgttaataaatgcaaatctcagataaagtaattcagtctttatcttatggttttaatttcaggatccataacaatgtaaagttgctcaaatatctaaacagaaatttaatatgtaatggtagaaagctagatgacctaggtgtttcaggaatcatatttctgtgttatgccattgttggtttctatagaattttaccctaaacagtttcagagatgctttcagataaaatgaagaacattaaattgactatctagtggcatatgtttttttttttaagtttttatatattttccttttttaaggaaattttagacatccaacaaaatggaaaaaaaaaaaaggtatcaagagttcccaaatacttcttgccccaacacatgcatagcctcccccaccatcaatgccactcaccaggtttctgggctaaagaaaatggcatttttcttccaaagtgtttttatgggtttattgtttctcctctactttaagggtggataagaagttgggtggataagaagtttctttaatatataacaatactgtttcttccatcacaaaagtgttccattttattttaccttaaatgcactgaaaatttcatttcaacaggttctattatcaacttatcaagcttattctactaccatttcttgttgtttggagtacatgaaaatgcatttttgtaattttccttaaataaattcatatttagggacgatctaaatgtgtacatcctgccaatgcatttagaaagttaaatagaatggtacaagtcttttgataattgatttgaatggaatctggtaaattgagatggagtgaaaatggtgacttttaataaggagaattattcattgaggattatgtgttcatctcccagatttttctgattatgagaatatttctcataatttcttttttttttttaatttctgatcttactgccttgctactaaatgattttaacagtgttactagttaaactccacaatttaaatataaaatgggaggaaatacataaagtactatgatgcatttcagtatataaaaaatattgaatccacatatataaaatatgtatttttggtatacttttatacaaaagtataaaactttattccatgggaacagcctcttctgaatttgttgcctgccttcaaattgaggtaggaggctagaaattgtactatcccaccgtgtcttcattaatttatgattcaccttctctgtctatccttcatgttttaaatgctctgatcaataagaattgactcttctaattctgtattggaagtgaaggattattcgagccatataaacacccaatggaaattagacttgcaacaatacaagctttaagaatcattgtttaatttgtcaaaaacacttattttctgatagtaataatataaactttaaggatcctttctttcatttgtcaaaataatacttattatccagcaactgaagagtataatctttgttggaaactgctggatctctgagtggtattttatgtttgtttctggcctggatttcagataccactttctcctccagttttagataaatgaaatttccaacagctgtggagatgacaattccagtcttttgttatttagtttggttttttccattggttctctttgacacttagattatccaaggcttagggaaaaaaaaaaagaagaaaaagtgatttttaatataatcaacatacacataaacccggccacctctttactaactccagtctgccaacttggaaacatgtaaacccatccattatttagtgtctgtttcattttcctccaaatgagagcatgttttttttttaatttgattttattggatcattcaaaacagcataatgatttatgctttatttatgatttattaacttataattttgaaactaaaatttatttcaagattttagaattgtgaatagtaaagctttatgctgctgtcatggatgtgcttttatttttcttataggaatacagaaacagagttaaggtaatgaacgcagcaattataggttcatggtttattaaaaccaaattccttattctcttcttaactactcatttatgtactttgagcctcattcatagccaatttactcttctctactcttggcgaacccagaaagatatacccttttgagttagcacctgtgttcccttacatcattttatgctggaagaaggcaatctaagtttaatattatctaaacaagactttgttgtactatcaacattgtaaaaccaaaaataaaccatctgggaatttaacactgtaaaatcatttgaagagacccttttcttcaacaactctgctgacatccagtatttagggtgctctgtatatcatagtccgtggaaatggtgccctgggagaaattccatgtacagcctacgcaagcatatccaagagcctcgcttaatttccattagcttaattggtccaatattgatcaccattgcatctgtcttataattcctcacacttcaaaataaacatttttttttattttaaactacaaaaatatccctactctttggagctctttatttttattctctagatttcaacttgaataaaactgatctttacccaatcttataacttaacataaatttttacataaacataggttagtgctatgagaaaaatataaaaggtcatgctgtaataaaataataatcttaaggaaaataatagagataacagttatttagtgctttttatgtagcaagctctgtgctaagttcttgataaatattttcttatctaacattcacaataaccctgtgacattaggtatatttataatcatttttacatccagagagatcaaaattgagaaattgctgaagcttattgggtaataaatagaagaccaggatttgaatccatatttgtctaacaatacttctggattcaaaaaaaaaatacttctggattcaaactcttaactcatataacctacatgtttttatagaggatgtttaatagcaatatcaatcatcatcattactatcatatacaaatatttccaatattccacaattgggatataaaaagcaaatgttgactccacttttagtatcaaaattcacatattctcccaaaaattcacctgcctttttgattgggtagctttgatctttgccctgtgcccatggcctgctcagcttagctcttgcatgcactagtattccttaccaagaatacccacattctttcttctctcaccaattcacctattgtttattcatgcattagctcagtaatcatgcatgattagtttctgggactagattcaagagttagctctcaaattttactcatttttcaaatttcagctctagttattccaacctaggataatttcattcttttttatcttctattgcactctggtcaaaccattgaagggactacaattgttttatacatttcacttttatttttctagctaaatatgcagcatcctgggaacatattatatgcaacatattttgtataccccctggaaaatgcctagtgttatagtattatgcacaaagcaaacaaacattcaataaacatttattgagtgattgaccaagatgagtgatattgtcatgctcaagaatgttagatggcgtgtataggtacatactaagaatttccaagctttgtatgttttcataaagttttctatatcagaaactcatttatcaaaaaaaaaaagaaaacaaacaaaaaaaagaaactcatttatcttcaggatgttttagttgcattctgaataccattctgaaatcaagaaaagttcattaagaataattctcttagctttgacctttgacttctgtgattacatcctccttctgactacctctgacatttttctctctctgttttttttcctcttttactaaaccccttctttctgctatctccatttcatttaacttcatttctaagagatggacacaattatctgggttttgttttcagtgtattgttcagaagcacctaataaccaatcaacccatgaaatatcatggcaaccttttaaaattttttcatttctaagaaagaaatacagtaagattcgatcaaatgtaatttatagtttgttgtccagttgtttgtctctaagcaattcttttggtatctttataacttaatttggattttatacttatttttcattcaattctatcagcctgtttcatagttaaaggtcttgttgacttaaacgtttatttgatcaccatacatttaactggtttctgtttgttcaaattggtaaacttcttaagttcacaaagaaatgagaagaatcaccaacgatggccatatacccattccaaatatatattcaaatgttacatgaaacgaaacggtgcaaaggatatctaggttgttgatattatatcttgaccttggattctgacccccccccccccagtttcatagaaagatgtttttaatcaatgttctcatctctaaacaggagttctctagagggtaccagatagccttgagcagaacagaaatttacatagtcatatttgaccctttcctccagcttttgtcatcaaagctgtaaaagatcttgttcatctcatctccttgatccttacaaatctgccttctttattaaatatagaaatccttcactagtcctcactatccttcaagttgactattgaaatagccccctcttcctcttaccggccattatcagaattacttgtgtaaaagacaggcaggaacaagttatgccccatttcttgggaaataaatacatacttaagaacttataaaagtttataaaataaaatcataatgcaccaccatatattgacattcaaatatatccctccatgaaacctaatcaaaccaattaggaagcattcaccaggtcttcctactgtctcagtcatgcttacccatcagtcaagatttgcttcctctctttttttgcagatttaaaccatacttatcttctaagattcagcctcattaccacctaccctgaaatgttctctgttctatccacctgatggaaatgatcacgtttctaccttatagcctctttcaagtatatgtatattttaaatgttcatttcattttgtcttacctatactccttttcttcttcacagttatacaatacttaagatttgggatcattaaattttcatatttgtataccccactctgtctaataccatattttgtgcatgtatgctctggatgttattacttacgatcttcgtagcccttgcctgcagtaaaacactttcactgacatctctaattcattttttgaaaacttttaagcacagttgccctatgcagatcatggtttttgaaatcctattcatgttgctttccattcttcacttccattttaagattcttctagtatgaattcatccaattctgttataattcccctcactagggtcaccagtgaccttgattgaaaaatatcactagtgaaaaggattataaaggaaaggagggaaagagagtgggaaaaaattagagagggtgacaaaacacgagagactcctaactctggaaaatgaacaaagagtagtggaaggggagtgggcaggaggatggggtaactgggtgacaggtactgagatgggcacttgatgggttgagcgctgggtgttatactatatgttggcaaatcgaacttcaataaaaaataatctaaaaatttttattttaattttttaaagactttttttattatttttttatgatagtcacacacagagagagagagagagaggcagagacacaggcagaaggagaagcaggctccatgcaccgggagcctgacgtgggactcaatcccaggtctccaggatcgcgccctgggtcaaaggcaggcgctaaaccgctgtgccacccagggatccctctaaaaatttttaaaaagaaaaatatcactaactgttttaagagcttttctttttcattcttggtgaataatttaatattgggatttacttgaaaccctctgtcttagtccattcaggttgccataagaaaaattcccaaaactggtgctaataaataacatttacttctctccattctagagactgggaatgcccagaatatggtcccagcagactgagtctctggtgaaggcattgttctgagtttatagatagccacctttcactgtacccctatacagcagaaatagtaagagatcactgtaggttacttttataatgcactactaattccattcatgggggccctttctaatgacctaatcacctccatgaggccccacctcctaatgccatcactttgagagttagaatttcaacatatgaattttggagtagacacaaacattcaatctaaaacattctctctcatgatttttctgatatccctccctctgggtttttctctttgcttttataactgtttattctaatatcctttgctgcagcctacaatatatacatcagaaaagtttgaagcaaatgatatatccaggaatgattatgatacaggtgtaaattaaagacaatgaagtgaataagatcacctagaagaaatgcattctgtgacaagaggagagagacaagtatagaacccaagaaaagccaatttgtaaaaaggattggacagtgcagactatagaccacatttaaaaacaacttttccttggaaaaatatttaaatcattacgtttttattgtgacacatcattttagtattgtatcaatatgtgaataagagaataaactagctatccacaggtttctttcagattttggattctataattttaaatgaagataactatttttttatgtttacagtttaaatcttaacaaaagaataatttcaaattttgtcattcccacttaatgaaagaaatggctaaagtcaaaccaagttaatggtccaataagtgtgcagataactcacagatgaagccacagctgtggttgatttgaaacaaacactaccgaaatagagcaagagtatatttacagagtcaggtgccaaacctaaagttcagagctgttggtaaagctgcatcactggcagagatttaaaatcaaaagcctttctaaaaaaaatggaacatatatcctcaagttattctatcaaaagtttagttcatactaaggaaaatgtccactttttccttctcacagctcatctattttattcttctttattcttttcccaatcatgggtgggtttcctatttaatttttacactcaagatttcaattcctattatacaaagaaaactttcataaaaaatcatgtgattctgcttttatgtcatattccgagacgtgtaaggagaaatacattttcttaaaagatgtaatgaaagggatgcctgggtggctcagtagttgagcatctgcctttggctcaggtcatgatcctgggatctggggatccagtcctgcatcaggctccccgcaggcaatctgcttctccctctgcctgtctctgcctctctctctatgtctctcgtgaataaataaataaaatcttaaaataaaaaaaagatgtagggcagccccggtggttcagcggtttaacgccgccttcaacccacagtgtgatcctggagaccgggtcgagtcccacttcgggctccctgcttggagcctgcttcttcctctgtctgtgtctttgcctctgtgtatgtgtgtgtctctctcacaagtaaataaataaaatctttaaaaaaaaaagatataatgagagctaagataatgtagacttactatgcacctttgaaaaaattgtctctgatattattattggtggctttagtaaaacattgttcattattcttaacatttaggaagcataatctttattacttccattggaatattacttcagattctgtagcttagtacattcatttgggataaaatataaaatattacggggccaatttgttactcttcaaacatacatgtgtcttccaactctcattccacagactgccatgtgcttgacagctggcaaaatcgtttataatcaacactatggaaagttgggcaagagttaagctacttataatatcttcagaatctcataacaagatttggaaaccaactgccatgtgaagattgtttttattcactctttcaaaatcaaaaaattatgatcaatttgtacagtgctagatgccaaagccatgtggagctttataaacctcagtcaacaagaagttctctttttagtttatctttgataatgaatgattttaatttcattaaacattgtatgctgcaaggactatctgtgggttggaatattaaattgagcatattcagctaatagattttattatttaagctggatttttcatttagagaagtaggactcaaggcaagcatcgagataattttttaatccaatctcctcatacaaaaagcaaatgagtaaaattttgccctgattatctgattatttcttaattctccatttcaactctaaggactggatttatgtagtgaagtaagtaaaatattgtattctaatcattttattattttgtctttatttcctatacaaagatgatagttgtgagaatgaaagttatctctcttatttttaacttattttccctaattgctaacagagtacttagcgcatattaggcctgcataaatatttaacaaatgagtgaagtcagactctagccttaggaaaaggaacttattaaagaattatctcttttttcctttcttatctctattatcttgtaaattatctaagtatttctaataacaaaaccacaaaaaaatgccacacttaagaaagtttatattccagcagaacttctggaggcacacagttgataataagttaaataattattggtttattattgaggatttacccaccaaattgacataatccctgtgcttattaaatgcagatctatattttgtagggtgaaggaaaacctaacatgtgcaggttacatttattgagcacttattatgtagtagaggtaatacgtgttttgcttacattatcctaatttaaccctctcagcaatcttatggcatacatattactattcacatttggcaggatagaatattgagatttagagttaagtatcttgcccgataacatgcagctaataaataatagagctgtgatttatccttagattgacttcaccatccatgctccgccaactgcaccacacagccaagtactaaagtgattacgtcagagtgccatcacacttgtctttgaagccaccattgatttcattcaatagttattaatatgttactaatctaatatatttttgaagataaatctgactgtaagtgtaaggaactcaagtaagaaacatttgttttttagtttaaatatttctaaagatcctctcattcctgaaaaaacaattaatctctgcctagccagatatacaaaatgtcaattttatgtaatatttatgaaagtgctcaaatctgtcaaacttagctactttttaaatttgggaagcttcttccttcaatacatttttgggcttgcttattcatttattaatacagatgtcatatagcttaagttataacacaaaaatgattccattagattgtaacctctgggatccctgggtggctcagtggtttggcgcctgcctttggcccagggcgtgatcctggagacccgggatcgaatcccacattgggctcccggtgcatggagcctgcttctccctctgcctatgtctctgcctctctctctctctctctctctgtgactatcataaataaataaattaattaaaaaaaaaagattgtgacctctgtgcacacacatatacccacacacttactcgtctctttattaattcacactgattttgagttcataatgtctagctttgtgaatatatgggacattttcacataatttctgaaatctttgaaaaccatccctgttatccaactagcaaaaacttccttgatatgattttaagagcattatggatttttttaatgtattcttgatatatcttatgaaacaaagaggtcaggctaattggaaacaaagaatggctgtattaaacaatgcatgtaacaccaaagcatataaatatcaatcaacatgccaatctccctggtaaaattatatgacttttcgtattagcatttgattgagtccaccaaggggaaaatatgacttaaaaataaaatgagcttctgaatcaggctcactaaaaaacaaacaaacaaacaaacaaacaacaacaaaaaacaaataaaactaaagagacttttattttcaggattccactataaatcaaagaccacattgtttctctggatagcattagtttaaaaagaaaaagaaggaaggcagaaagaaaggaagaaagaaaataaattcctttggggtcccattggccaaagtgagtgaccaaccaaattgatgaagtcaacatgattctaaatttaataatgcttcaatatggatgcttctaatttttaaagaaacattactaatgcagagcaaatggactaatcatctcagtttttttttttaaggattttatatatttattcaggaaatacacagagagaaaggcagagacataggcagaaggagaagcaggctccaggcagggagcctgatatgggactcgatcccgagactgcggatcatgccctgagccaggggcaggtgctcaaccgctgagccacccaggggtccctcatttcagttttaataaaaacaagagcataagtcatttgaatgatcacttgcagttcatggtaaccaaatgtgacaaatgttaatgcaaccaatctccatctctttctctagatgtttttgcacaaatagagctgtaattactattagttacctttctctaggagcacaaatatgcttcactactgtaggccccatatctccttcagagattataaataaatactagcaattcagagataacaggtttctgtctttttcaccaattctccgttctaagtatagtggaactgatttcagaaaaacaagcataaatccccttactctgtttcttttgtcttccttttttttctcaaagatggaaaataagccacttttcaatttcactctcactgtatcataatacttgatgtaattaagaatgtcttttgaactgataaaagatacagaaatgttaaaaagagagctcatggtttgttctttttgtttttgcttttttaaaagattttatttatttatttatttatttatttatttatttatttgagagagagagagagagatcagagcagggcagagggaggtggagaagcagacaccccactaaatagggagcccactgcagggctccatcctaggatcctaggaccgtgacctaagccaaaagcagacaactgactgagccatccaggcacccctgaattctcatggcttttaagagacatagttgtgttcaattttattcaagacttacttattgcacctaaattttatgactgttgttttgaatcttaatgtagtagcaataaatttcatttcaaaattaatttattatttattataaatgttctgttgtcacagttatactttaatgttttgcgtcattgaacacttgtagttggcacaaatatactagctgagatatgttttataatcaaagtatttaaatataaactaaagcaatttcttatttcgactggtattaactttccatttggtttatgtagagatgcacatatattaagattaatggatattattaaaaataagattcattttttttcagatcccagaagttaacagaaataatgggcaagaaaatgactatgaaggtgatattacctcattatttacgtattctcccagtaagtgttcttgggtgttatgaacacatgtatatatgtaacagctatattaaggtacatgtaactgataaagtaagactgaagagagtataggagaccatagtgagctggaaaggcatttaattagctgaatgacttttcatttataatatagtaaagatgttactacattgccttgctaatctaaatacttctactaagttcagtgttaagtaatcaaacaccagtctttcctacgcagagagaatacacaagtcagggtgatgcaaatattctgactattgacaactctgttttttcagtttttttaaaaacaaagtgactttcttttcagaccttagttccctctgctaggaaatgagaaacttggactacaactctctcgaaggtcctaactggctataataggatgtgattctgtattctaatgtatgaagactaatgctgtctttctgcacatatgtaactctacccattatgccactggtatacacagcttgctcagcatcttcaccagcatgtctgatagacatttcccagtcaacatttccaaatccaaaaccactaatatcttgaccctttcaaacctataccacttgctgactttcccatctcagtaagtgaagattctttccttgtttaactcaatcccaaaacctgggcatctcccttggctttttttacttcctctctcaccccacatccaatctgtcagatgatactgttgattctaccttcacagcatagccagcattcagctgttcctcactctttcattatttatcctggtctatgctgccgtcatcagctctcccctagattattgcaatagcaccatggcactcagtcagggtatttcccattcacaacagaggatatttgtcaatgaacagaagttttttttataatcatgacttgagaagggaggtttacaactggcatttagtgggtagacaccagaagttctgcttaaatatcctacgatgcacaggacataccccacacgatgcacgattatttgatccaaaatgtcagcagtattactattaagaaactgtggggcacctgggtgacgtgattgagtgtctgccttaaactcagatcgtgatcccctggtcctgggattgagtcccgcatcaagatccctgcttctccctctgcttatgtctctgcctctttctgtgtgtctctcatgaataaataaataaaatcttaaaaaaaaaagagaagaagaaagaaagaaaagaaagaagaagaaagaagaaagaagaaagaaagaaaagaaagaaagaaagaaagagaaagaaaaagaagaagaaagaaagaaagaaagaaagaaagaaagagaaagaaagaaagaaagaaagaaagaaagaaagaaagaaagaaagaaagaaagaaagaaagaaagaaaactgtgttagcttccacatttgccactcctaatactctgcttcatgaaggaactaatcacttttctgttccaaatttcccaataccttcccttcatatctatccttctcatcatctaaaaagcccgacataaccttttcatcccatctctgaaccactctcttgtttgttcacagcacagtaacatctcacatatcaagcatgctcttgcctcaggacattgcccttttaccaatgcacaattgatattcagctaaccattattgaccactggtttctcacacacacggtaggcaaattgacctcaaatcaaataaaattcaatacaatgtctaactagcatactgtggcaaattagtaaacaccttcagaagttatgccataacaccaaatagtgctaaatattagagtaagaattaatgtgctgatttttaaagtcataaattttattctcatttttaataataaaattgatgataaaggctatcattgataatgaccatttatttattatgacatttaaatcctcagaaggactgcctataaagagaaaaatgttatttcaattatttatagagagcaacctcatttccatagatcatgggacctggaatttgtctggtatatagaattctgtgtgactggactctcaggcctcaggctctttactatcccagggatgggggtgaagcatagccaccttagttggcagatttccattagtatcaccatcactgtgaagtttgctaaaattcactgagccaccacatccaactctccaaggctgtattctaggtctggacatgtttggcaaggagcaaggaggttctgttgtttaagatgtaaacactgaatacataaacataggtggctacaagtgatttcctatagcatgagtgtaattctaagttgatgatcaatttgacagaaatagtgaaattcctttactatcctgagcaataaatccttttcagccactacatcatctattccatcagcatttctagagactactttatttcaagctcagtgtttcacattaaaggtagaaagatgaatgagatctgtgcaagagctctcattctgttggttgttctgctcaggctgctaattaaagaatcttggtctgcagttctagtactgaaactcaatatctttcttctcacatgaaaggaaagaacaataaaaaattaaattccatgtagaaatatgggtaccatcataggcacaatatttccttatgactaattttttctcataactgaaaagtttctacagagtaactctttattggttaaaaaaaaaaaaaaaggtactttttctccatagtctgccagttggcttctttaactatatttcagcttagcaacacttcaaaacaataatcatctgcttgtctattgcactgacaatgacaacaattcacatattccctccaatatagggattccaagttcatcttaaaattttgtgaatcttttcttaaagcagacttttcatacagaaaatttaaacaaaaaaaattgtaagacttaaagttattgcattataatatattttttcaattcttcctactaaaatgtatttattatgtacactataaaaacaaaagatgtaatcatgattcttaagatggagggaacttgtattatttggttaagtttcagaattggatatattttataggtgactgaactgagtcttagaaagattcacctactgttggcgatttggatgccttctccaccaaaaaatattaacacttccctcaagtaatatcttcaagaattaagatacaatctggtgataattataattggggatataaatggtgataattatattatattaaaataataactaagatcacagattcacttttgttgcattatttacaagcaagtcaaggtttattttattctttgtatttaccttttccatagcttacttattacttgttaggtcagttatactgtattctgataaaaaaggaataatacagacatatgtatgaatctgctcaaacaataaccctagagaatcaaaatgttattgtatgtatgagtgagtcattatagaaaggtagaaagatataatgaagctttaactgttcatttggtaaatattttaaaagcccaagaatgactattcactaaaattatgcatttgtcagctctccaccatatcagatcataaaagttttattagcagtactaccacttctgtacgaaattagaaaatacaaaatgcctccagatggtgtgtttaaatgaaatctactatgttcatcctaaacagccatacagaacccagcatttcatcacgaaagcaggcagacatacaggcaacgtgcagaaggagctgaggcagatgaattggcagaagaaaaccagaatgaatcaggcacagagactgagcacagcttagcagaggctggcagtggaagttctgtaaaccagtacgatgatgacttgtatgtgttcattcctggagacaatccagaacataattcacaagagccactcatgagcaacagaccacctctccccccgccacgacctgtagctgctgccttccaacctcactgcacattacagggtaagtttaaggtttaaaacaacaacacgagtctgacgggcttccatagtgtatagtgcagtgacaaatctgttacagggaagaccaactttcaaaaaccaagagcaactttcctactattccttaactaaggcctcagaaatcaagcaagagatacctgacactaattcacactccctcctctccttccctatctctctctctctctatctctttctttctttttttctccctcccttcactctctctctctctccacctttctgcctccctttcttctagaacagtctacattacaaagctttaaagaggtaaaacaataaagcttggaaggagtttgtagtgctccacataaaatgagcctcaaacatcatgtggaaaagtaaatgacaactttccttttaaaaagtactcctagtcgtctggcaaaaggaaaaggctaatgttttacagatttgaaaatgaccttgggcactttactaaactggtagcaacaggggaagcaagtggtaaagggcttattgcacactgccttgagaccaaagtctaaggacactcaacaatatattgctaaaatgggagaacagttaaatggctatgatcacatttgttcccaaatgcaagaattacaaatgtgatggggagatgggtggtgcagttaccttcaagaggaccttccatattgggcttatctggctggagtgttcaacagcctgacagacttccagctccaggtttacccttacagtcactaattatcatccactttattgatcactattttatacacactttccacattcaaaaagtgctcttacaatgaaactttaatgagaaagatgaaagaaatatctattattaagattccatttgtcattatacttcacagctgttgagtcctagagggctataaagtgtataaatatgttcaaaaaagtgaattttgaaaaacaaaacaaaacaaaagcaaaaacaaaaaaatgaattttgaagccctgaaagtcagcatttgcatcattgaaggtcttatttctataaaggttcaagttgatgaacataataaaccttttactaaggttatacactgtatttagagatgaaaaatccttcttactggaaagtatggaaaacctaatgggtactgctctgattctttcacccattcaacaaatatctatcgattacctactatgtgccaagctcttatagtccattgtgatagatgagtgaagtaggttaatcaatactgggggtggagagagaaacatagataagcagtacactgacaataacattctgatatattttctcacttttttacagagggaataattttatataagtattaaaataaagtgttaagaaggcaaagttgtcttttatctggctaaatttcttaaaggagctaataatgaatcatattttccctaatgtaaattcttgtatatagtatgttggttaggaggtctaaaactactctgtagagagcattcaccagatatctaaaaccagaattgtaatgtttgctaatatatcttctgaaaaataccaaatacaaaaagcaaaataaaacaaaaatccccactttttattttaaaactttacttttacattttctatgcagtgttacttttaatctcatcttttcataagggctaatattgcaagtattataaatggataataaaatgtcagtattttttcttaaaattgtttgattaaggtctttgaaagtaagactagaagctaccactgttttgctttgttttgccagagaatttgtatggcttctttgactttagtgataccttcagaattgtcttctaggactagtttccatgtaccctcaaattaccaaaaaagagaggtaacttcctcacgtcctgagcatcagattccccctaatggatttatgggcatagtcactggagaaaacaataatataaaacactatagaaaactttttagtttataaagaacacattgtccatttttatcttcaccacacatttgcaaggcaacaatcactaacatccctcccttgactcagacccaaaaccacagaccctgtaattagaatgtatcaaatacttctgtaaacaggcttcctcagcttttaagattcttccttttattacacgtcttactctgttttttttttcttcaaaaattagaatgataggagtgatttagttttatctttatttgctaatgtacaacttgaattaagaatgctcagaccccactctgaggtgaaaagtagaatctgatacattgggaaatagaaaagtaaaatgtgttatttctggggcaagcccaactttagaaggagttgggaaggaaggtgggtggagaaacaaagggagttaacagctcaggaaatgggttgataaaagtaagatagttgacacattccgctaaaaatgaaacactgtaaaaatgtttcccacacacttaatctggtttcacctgtttcgctgtcagtgaaacagaagtcatgaagtcaaaatctcagtaccatattactcacagggagagaggcaagtcgtgctgatttgaaagttagtgctaagcatttaggcattagtacttcttggcttaaactaactgaaacaatcagatctaagactctattgggggaggatcttggcctctcctcatgagcctacccaaaaccattgctgacctacttaagataatatacttttattccactctacagcttgtcttcttagtttctacatttttccactgaaatacccataagtaacctaaaattttttggtcagattatcaccaaagaggaaggagaatagttctccctcatttcgtgtgcatgaatttctcagaacaaccaagaaaggcaaggatttttcactcgataactgaaattctttccattttattgttttcattaatgctttaagtaaaacatctgtcaaaggtctattttttaaaatattttatttattttagagaaacagagagagaaagagcatgggtggggaggggcagaggaaagagagtctcagtccaactccatgctgattgcagaggccaaagtaggtgttggtcccacgaccatgagattccaacctgagccaaaaacaagagtcagatacttaaccaactttgtactcagacatcccagaagtctaatttttaataaaattctcattactgtgttagagagctatcaaaaaaatttccaacttattcattaaccaaagtaggtgataccttagtctcctaaatactactaaaatattttaattcattattttaaaaatatgcaaagattttccaagtattaaaaccataaaatattattcttttcgtatgatttaatcttcaagtcattgattatcctttccatcttgtcacaaaccctactttcagtgttctttatctaagaataaggttcatgctggcttatagaaatcaagagttgcatttttgaagctcagtttgtcaagccacagtgagacttaactgtaagaaaccaagtaatggaacatttggctattcctcctgtcatcttcagagaaattgaatgttaggcaaaatacccagtgcaagcccttttgaaaggtaaaatgacttcaagtgaaaaaaaaaaaaaaattagcatattctacttgaaaaacaagttttcaaagcatttggaatttgaggcttagttttatcactcaattttatgatactaaatcccttataaactatgtggcaaatacttatatgcttatttatatgttttcctaaaaggttgtgttttaaacaaactttattaagattttatttttaagtaatttctacacccaatgtggggctcagatttacagccctgagatcaagagtcacatgctctactgactgagtcagccaggcaccccaaacaaacttttttaaaacaaagctaacaattaaaaattgcaggagaataggttaataaattatatggtgtacttttcttttctaaatttatgaaacaatatgtaaattatttggggggggagtttcaagaaaacaatagatggacatgaacaatattgcatataatggaagagtaaccaccatctaaatatttcataagcagggaaaatggtagaaggccaaatggaaagaggtcagaattgctgtgatctcggagcaagacaagaaataggaggtgaacccaaagaagaagaaaagaaagaagatgaaagggagcaggagaaggaggaagatccatacacatttgctgagattgaagacagtgaatatgatatgatactagccaatatgagcacaaagaaaaaaattgggagtcgatcttttattataaacagacctcctgcccctacacctcgacccacaaatatccctccaaaagaagaaaccacaccttacatagctcaaggtaatgttttttgttttctgtatgtgtgtgtacgtgtttggggatcagagaaattgtcctccttttagtggggtcaaaagccccaaagatgaaaaattcttttaaggtcataagtgagttgtattggctcatttttaatttagaaacacaataaataaggaaaataaaaataatgggtaaggttctggtttctaattcttcgaaggatagttttgataaattatccgaagaacatgaaaccatgacttgtgtggagaaattctatttggaatttttcctataattctataagagggaagctcaattcctatgaataaatagcaacacaggaaaagaggtattccccttactttttgccccaactctattttttgctcctaattccacttaaaacctaaaaatctctgagcactagaatcccagacatgcaatcccaattactctgataattgcacagcaccccaacaattaagattcacttcatctgtcattgaatttcaacttcattttagactctaattcttctttctcattttctactcagttgaacctaaataaaaaaaactaacaaatccctgagcttttttatatctatagccccagcagggtatatttacactattgttattttctggcactcttgtatactctgttatggtttgatgtattccacaagtccatagtgtcttagagatgatttttatttatcagtacattgcatacatgtgcactgagtgcttttttggactaacataccatcccagaggctgagaaaatgggataaaacaatttcttcattcttgaagagttccttgtggacagatatgcaaataaatataaatatccagagtacaaggggacactttattagagacttttttaacaaaatgtttgggggttaactaaagctgagaaccaataggtgatacattcaaacaatttaaaccaagtacacaaacagagcatttaaatataaaccacaaacctacccaacgaagcaattactcgtctgtgcttagtatgttcaaaagaaaccattcaatcgcttaagcattatgtattagaaatttgtacaaaaggggcacctggggggctcaggggttgagcatctgcctttggctcaggttgtggtcctggggttctgggatcaagtcccacatcaggctcctggcagggagcctgcttctccctctgtctatgtctctgcctctctctcttcttctctctgtgtgtctctcatgaataaaaaaataaaatcttaaaaaaaaaaaaaagaaatttgtacaagaaaatttagcaagtatatgtaagtatcagtgcacaatatatttactcaacagtgatctactgctgataatgttcaagaaactatttaaaatcagaaaataacaaattaataaatgtgtcagctgaaataaaatggcaactggaataattatctagattattaaggcagggaatagctataattagaaattcttagattgtctaatatagtgacttctagtttgctactagctcttaggcagggatactgtctcccactaaatattttttaaagacagaatatagatacagctcaacacaagaatttagttgtattaaagaaatttcaaaacaaaacaaaacaaaaaaaaagaaatttctacagtggcactttgtcctctacatcccaatccttggagaattctcaggaccacaagtagagactcccatattggggtgttttgttttttgctttttatcttcccattcttctatcggctgtcatattcttattttccatgaactctttcttgttctctatttgttttctttttttacatcattattatctcttctttggcagtgctgagggcagtataattcattttctttcattttgctttgatttttcagatttctgtgattttaatttttctgcagttttcatattatttctgttttcttagtttgctgtttatgctgtctagaatgcttaatacttccatcaaatatcttagctgtgcttttatgtgtaagaatgaagcaccaaaaacctaaaaggaaactcaacttgttaactgtggactacattgtagggtaatggggaggtaaccaaattttgcattgaattaccccacattagtttatttagatctattttcctatgcttttcagtttacctagaataaaatccaaaactccaacctactgtatatatctggttgctgtaactttgagagatcagagcatgggaagaagactgggaaatttcaccatttagaatgcacaattcaagtccctttgttcagtccagtgtataactcctgggctcatggtgcctcatgtttcatggccagaactgctttattctttctccaaaaaataaaccttccatcctggccagttgcctcagttcattagagaaggaatttggactatgactgctttatacttacagctaatctcatgttaaataagagaattgtgatgatttttcacttaattcatttatttattaattacataaagatttattgagtaccaggtgataaaatctctgtgtgttaagtaaataccttacatgtcaatggtacatttgtcatacacacacacgcatgcatacatatacacacacacttgcatacacacacatacatatatgcaggaactctaaaaaatcttagaaatcttgagtttctaagaaattttaaaagctatttatcttacctacttattgcacatttgacacccttcagcatttataagaaatctttctaattcaattacataacataaattactctcactacattcatgttatatgcagacagcaaccagtgttataatatgcaggatttttgttttcatattttttgcagttaagtatataaaagcagttacaggttttggctccatgtcccaactcaatagcaaaatctgaactgtaaatgaggtcaccaaagttcctttccattatttcttcctttcctttttcactgggatatacttgcttgcctcttccttttatgttattgattgttgaccattctgttcccacattaggaaaaattgtctcatgagctaattctttcttgatgttctttgcatttcacagggtaaaagtcttccatcacatctttcctctttttttttttttttcatagctcaggcagctgtattaaataatattgccatcatttcccagaaagtcatccattcaattagaagagcattaatttgcccccactcttgccggaacacagaagtagtagaggatgttggcacatccagaatatgttaatgtagccaaaaaagaagagtgtagaataggagatggaagaggaatgaaagatgcagaattccaactacttcagaaatgcaaggaagtactggtaaggagactaggcaaataaaccagagagatacaggaaaatcatgttggttttttgcaagatagtagaaaatgtgtgtattggtgtccacccccagatcctggcacagagctcctgaaaccttggtaacttcctggatgacaaggagtgttttttgctctaatgaggcaactttgaataggcttttgaatggctcctggatgagggttggtcatcaaatacaccaagccatgatagaggcttagaatcttcagccccagacccccattttcttgagaagggagaagggctgaacacaaacttcttggtcaatagtacctatgtgaggagggctccataaaatcctaatagtaggggattcccagagattccatgttgccaaacagatccacacaggaaggatggcatactccaactcacccattatgcaacccccctcacacacacacacctcccctctgtcaaccatcagtttgttctctatatttacaagtctgattctttttgttcatccattttttttagattccacatatgagtgaaatcatacagtatttgttttaacagttaaaccatcctatgtttgaagtactactaatgtgaaaaggggaattcatacaaaatgggacaaagttctaggatatatattgacatagagttaagcagtttaaccactttcacacagcataacatagcagaaagggattctatttccaccattgcccccagaaatataacttattaatgcaaaactaaacccagggttcattttattaagagctgtgtttctgattgtttatttcattcctgttatactaagcctaattttgggtgagcaaccacaaaaagaataattcatttttcttaaatgaaaggtttttgaagtccacatgctttcagatgttgcattctctcccacctttttctccctatgatatggcaaaggatcattcattaagttttaagaatatggaggaattaatatggatttacagtcctccttaatgacagaattgttgattaagactaatcaaggaagaaaatgaacattttggtagtgcatatagattggtagtaaaagaatatccataaatcatgcaaagagatgactgcctattggggaaacttttgcagcatgaatggtcaatttcttttttttcccctcacacaattagtagaaccaagatccttcagagtctgtgttgctggttttggggtagaatgtaatttccctttccattttcaattgctactgagttgcccttggcattttgcagagcaacaaagttgtatatgacctttttgagagctactcaactactggggccctaaaatgtcaagtacaactcagttgctctcagaaattttccacttgcaattcatttatacttgaaattgatcacatacaatgtaattgaaatgcaaacagaaaaaaagaccttgtaaaatctcttaggaaaattaatctgtatcattgtatatgtaacagaatagaatggtctctgtcctcattagtgaagaaatgtggtcaaatggattaaagtgggccttaggtcaactgccttggatcctactctctggcagggtgaccttgtccgagccatttagcttcaggacttagtatgtatatcacaggtgctttcagaacagaggggacatatttattctatttgtaaacccaatgtccctgtatacccccctgagattctctgccgctcccctttccttgataaggtgctggtacaactgttcagacctgtgctctcactatccttcagaaggaatcaagcctggctatgtaacccagggtttttgattggttctttttgtctttctcacaatagcatatcacagaaacattgaattaaacctatgaagggctttactgaccatctgttagagtgttggcataataatgaagaacttttccagcctcatcttccagaaattcaattccttcatgttagtgaagggagaaagagtaatcaactgtggtgtaaattgacatcaaaagatttatgttacatgtgaaaataaatgcaaatattatcaagtgcttcaattttcattcaacagcagagtgagttctggtctttctccccttaaattttgatgcttatagcatctctgttaaatatctctttagtaataaatttaattggcctagaatactagttcttaactggtgatgatttgcctcccaggagacatttggcaatgtttagggacattttcagttataaccactgggacagcactaatggcatctagtgtacagaggaagggataccactaatccttcctacaaggcacaagaaaggcccctcatgaataattacccagttcaaaacgtcaatagtgccaaggttgagaaaccatgacataggttgtgcccattgttaagacaaagtaaacatctgaatagaatacttcagagaatggtcatttgaacattaaatcatagatgtatcaaatagctacccacacaagttgtcttttttctcacattagatttcctaaattcacaatattttttcaccagcactttcagtgtgtatatcttttttcctctaaatatgtagcagttggagaaaaataagatgttctgtggggttggctcactgagaagaacctcctctgagggattggcagcatggtggaataaaattcatgaagactgtgaaactggatgaaacagaatttgaatcatgacttagccacttaaaagttgtgtggctttgtacaacttacatagtcttacttcattaaaaaaataaagaatgataaaacttcctgcatagaattattcatgggaattgaaatgacacatgtacagtagctagcacagagtgggatgcaaaaatggtatgaaggagagtattctgtatacacggacatgagataggacttgctagatcttttcagtcgttgtgttttttgcttcttgcctgggtttcatatgccaatatctcatcttgcacgactttttcaaggtatttattaactatcaatactgaacactacatagaacaagtgggacagccctgtccttttagagttttcaatctgcatttctgtagtttgagtcttaatagcaaaattagtatttaatgtctgttaaagtaagcatgaccaggtccaatggttacctcttaagatagcaacatttcctaataaagggataaaaagagaagtcacagaaattcttttgggtaatcaccaaccatgaaccaaaagatgaactcatcatagtttctagtgtattatagaggcttctcaacaacccaaaccatttctcttaaaaaaacgaaaggaaaattactaggatgttactggagtaatatcttcatttatatcatcattgaatacaaatggtcaaaaaaaaaaaaaaatcagccagtgaacctaaatgaagtcatatgggaggagataaatccccagtccagggctatgcaagcaacaggtgttcaatacatgttgcctgcctggtaacaagacgaaggaggacctgcctgcccagtcatgaattggagatttcactaataaactcgttgcaaatggcctcaagctctaattacacagaattccataaaccaactgctctgtcttttgaagtgtctcaacttcctgaataagaaacatttctagaatagaaatattcttatttgcatcaaaggaataaatatgcaaataaaaacagtttatattctactctagtaaaaattaatttccttagacacggaaaacataacttatagtaaggttttctgtttgggaagaaaatttttatgggaacattattagacttaaagtttccattacaaattggaaagaaaattcatgctttctccttaggagaaaattgatattgccattgaattagagtgtttatgccattttccttctggtagtttatatgatttccaactttccttccctaccaatctcagaagtaagcttaaacacttcagtgacctcctgtgtgctaattgaccatttgcagtattctccttaaaaaaaaaaaaaaaaaaaaaagaatagcacaactcctgactacatatatctcccaggcagtaaactagttttggatggacagccttcatagcatgtggctgatgtgactatctgtggccatgccaaccctaatgtgcctattcaaatgtggtggcctatatttgataaatttgtttaaattaataacctataactattgctaacaaaatgcaatgatggttttaagccaattacagtggtcaatttgcaaagaagctacatctctcctctgagcttcagtttgattagccagtgtgacttctgattgaatattctcttaacctgtttttgctggttagacaaaggagctgtttacacattgtggtttggtttggtttggtttcgtttttacctcatgctacctttttgttattacctttcacagggtaatattgggggaacaaggtaggaagagtaagtctactagagtcaagttaccaccctagcctccctgattgttttttctcatctctatcttctttcattagtaagcactcatgacacatttgacctttgcttaaatgaaaacatcaaaatggaggacagtatggaaactctgattgggtcttacttgcctttgcctaacactgtagttgataacacagttagtaacctaatgaaagtttattgaatgaataataaacacacagctgcaacctgtacaatatcattccctgagaacccagagcacaatacaaatctgtatacttttttatatcaaatctctagtttcaagaataatcttcagtgatagctctaaacattttgtgttgcacttctaatgggcatgggacatcaaggggtttgttgtttataagccacatctttatgaatgcatttggctattgcacggactttccattaacttagccacttacaaagaaattccaaataagttttgtcagatgttttaaagaaggtggttttggaagggtcccttcacttaaaataggccacaaatgaagttgcatgaagaatatttttgcaaaagcaacctaggaagacaaatagtgccagagaatttgttatatttcaccttctcctcctcctctgttcttctgatagttctgctttgcttaatgttttgaatatttaatactatgctaatttttccatttagtgtcatttctattctagagaaaattttatagaagttgttctgccaatatttaagataaatggggatggtatttgggacgtttgtattgtttgagattattttgaaccgcataaattgtttttctttccctgtggactttttttagcgtttaaatgtagtgacaactactaataatgtattcattttcctttcttctcaaattccaattttaggtaggaagagaaaaataagatttcactcgggcagagtcatccaagttgatgtatgtcataattgccttaatagcattttttgaacattgatgaagtaaaatatgattgtaaaaaatagtgtcctgtaagaattcaaacacattagggacaaaatcacctccatattttataaaaaattaccttggtctatgatttaactttctagtttttctattattctaagctcttggtacaaataatctcttccacaaaacagagcctcctatttaaattcatgcttaagaatttatccagcactattgagcctggagtgacttgaaaaataagaattggtagcatcttaaaatgtcagagctaagtgggacatcagaaatgatctcatctgagctccacctttatcagcaaggaaataaaggcccaggcaggaaaaccattttctcatgtcacatatgttaacattaaaacaaaactatactctttccattgtgccacgttggttagcattctggcatatatgcattgggtttatttgttttttccaatttctgtatgtttaaaacagactctcatcttaccacaaatcttgtacaaaagtatatacaatataaagctgagtgacccataactcaatgttcaattctcaggatatcatggattttgcatttacctcttataaaggtttgtgtcagtgagcatggtaaatagaaagtatataaagccctttagaacaaaagtattagtagtaaatgttaaatgtttcagctaattattaactgtaaaaaactgaaaaattagagaattctgacattttctatctatccttttcctaaggctaacagagtatgtttactttgttatgcatgttagagtcagtgatcattttgattgactagctcatcatgagacaagttaatataggtctttttccaagattacctcatattcaaaaacacctcctaacttccatagcattgagaaaatagtgtgtttgtcaatgtgctaattgcgtctttgtaaagaaataaggattgtgtgtaagtggaaggaagggaccagttgaaatgcatgcaggtgaaggtaaattaagagagaatttcaagagagattatccaggtagaaagggtctttgtactgtgggtgtgatgtgctgcacaccctgcctttaaaaagttctgttgtgagcactttgataaccatccttctcagacacccaactcccctcccagtcttctgtcagatcccaagactcttccaactagaagccctggtgaaatctcaataagaaataaatgtactgtttataattctaaggctagaaactgaaagagaatgttgactttgtagtgtttactgcatttcatacacactaggcacttgtcagcaaaaccacatataaaatgtaaagtgatttttaacaaagtaatgccaaaaatctggaaaaaccaattccaagtacagcttcaaaatgcaacataatcgttaattccaagtgcagcttcaaaatgcaacataatcgttaaaatcaatcgatcgatgtactttatatttggagaatttgttgtctggatttatctttggattcagattattttccatattaatgatggcagactacagatttttttttgtcttagatattttcagttattatcttagtatcagcaaaaaaagaaaaagcacacaaagactctgcctgttattaaaattcaaaagtgtttcattttttcaagactttttagagctagccaatggaaaaaaaaatcaagcaccacttgtaaaaatttttctaatatcctaaaatatattcacataccatacatccattattatattttatttaaggaaaagtataaaccacaaacccagaacttgccattaagctatatttagcatgttttccaaatactaatttgaagcagaaaattgattttcaaccagaaatgttatttcatgtgtataatttttcttttttttttttttttttttttttaaattttttttaaattaatttatttatttatttttattggtgttcaatttactaacatacagaataacacccagtgcccgtcacccattcactcccaccccccgccctcctccccttctaccacccctagttcatttcccagagttagcagtctttacgttctgtctccctttctgatatttcccacacatttcttctcccttcccttattttccctttcactattatttatattccccaaatgaatgagaacatataatgtttgtacacatcatactttgaaaatatatttgcttttcctgtttgaaagaggacagtactctttctcttgacaatactgacaaactgcatatgcttaataataattttgatgtgataggatttggagcaatttttaaaaatattttatttatttattcatgagagacacacagagagaggcagagacacagggagagggagaagcaggctccacgcagggatcctgatgtgggacttgatctggggactccaggatcacgccctgagccaaaggcagacgctcgacccactgagccactcaggcacccctggagcaattttttaagttaaaataaaaatgtcatctggattaaacacaggtatgaccttgacaatagattaatctaaagaatattaaaactaaagatgaaaaatataagtcaacttctaggctgcatctttctattttttaaaatttatttgagaaagcactgatcagggagcctgacatggggctcaatctcaggaccctgagatcagtacctgagccaacggcagaagcttaactgactgagccacccaggcactctgggctacatcttccaatcagagtggccaagcaccatccagctggacggtgctctccctcccacatgccacagctggattgtcccaggagagtctggtgagctgctaagggaaaccagagactcaacaatgctgggaatgctcaagggttcttttggttcccctccttctatcttcttctttatctttaaaaggaaatagttatggaagagaagaggcagcagtgctgtggagaaccaagcaatagatcaaagttaaaggcttgaggctctaaatcacgttctctactaacaacagttctgggatcattgcgaaattacttagcccttttacaagttagtaaaacctccacctccctcactttaagcatatgaggttggactaaatctccaagacctctagcagttcccaaaccctgtaattcagactacccataaaaaataatggaacaaactctgggatgttcaatttctaaagtcaagttgtaaaaggtataggctttttcattaacaaatgccagaagctatacggaactttgtgggtgctcaataaatattaaacaatgaattttatctttcatctgcaaaatgggatgagtaatgctgacttgtctcacagaatttcatgcgagaaataataaatgagaacaaagcacttttcagataatgaagtaattcgaatacgctgctttaataggtctgaggtctagctagatgatgcaagaataaagtgtctttttcattatcacatctgtgaaatacagcagtcttttcatctgtttaagaagaaatagtcattatccttgcttatcaccagaaagtcagcatgagtaatttctgtgcaaatggaattttgcatgtgattttaacatttatcacttttaacaaagtgcaccttttatatatacctgatatcagaatggaatcatatcttgatttaaacagtttaacaaggtgttccatcagcattgcactgttccgcctaaagaaaatgctttttgaaataactgatgataagctgtgcaaatgagaaaattgtaaatttaatgatcctcctactcaaaaataatacaattcaagacttgctactaaggaaaatgaggtatcacactctagtttcctagagacttccacagtttgttcttttttatgtttgtgttcaataaatactatgcactatttttttttacagtgtttcaacaaaagacagctagaagacaatctgatggtgacaagttccatggtcctaaaagacaaggtactgacatgaacttcagtttatttaataatctctaagtgagatttaaagaagtcttataaataatgaataaggtctccaaggatagaagtaaataattttgtgcctgattagttttactcttaaactagtgtcttgcccaaatataattttgtcttaagattaatacaagaaaattatttcaaataataaaggcaagaaatagccttgtgagaatgtttatgatgatgttgatgatattattaccagggctctaggaaggtcaaataaaacaaattctctaacatgcatggagaccaggctttcacagactcttagagagaggcttctggagtttaagttgggagggttgggggcaaggggacagatgattgattcccccaaagaagggaaccagcatgggatttctgtactctgcattgaactccaatcagattgtacccttgtctcactattctcttgccctcagcacttgatctagcatttttttggaacacttgtttgtcatgcagaaaagaacttaaaactgtagctccagagacatcacacatagtagaaaaattttcctctcgctctacaaagaggacactgtctcaagttttccctaacatatctggttcctgttgaaaagagaaactctagcaggttgtttattattattctttcccctgataataagagcttggttaaagttcaaaaatcaaaaacaaaaggtgacagatgtagaaggctggctctgataatcaatgaacacgtgcagatgcttttaacctcaacgcaccgttgaaataacttcaatccaaaacagcactgccttgaaatcgctaccagaaaattattgagagagatactaaaataaaaccaagaggttatctcacagcaggtggtgtttcatggctaaacacctagccagtgtccctttgtgccatcatagagatggttaggcaccccttgcccccactccttaccccaccaagctactaaatacacaatctcagtagaactgctcctaggacatcatggattatcctcaagcaaaggtatgtccttaaatgggtgttcttcatctatcacttcaagtggcacttcccatggcagcaaggttgtacttagatcatccttccaattaagtacatgatgcttgaatgcagcacatcatgaagctgaatttctggccctgaacacatagatatactgcccacgaatttacatgcaaaaaaagaaaaccttctatttgacaaccttccatttctagtgatttcataatttcaggagaaagatttctctttttaagtctactattatccttccactgaacctttacctctctcaagatataagtgcagacttccatatgagatcattctacaattagaatcaaacagttcctgaggaattaacaaagtagagattattcctactaggactccccatatacctcacagaattttggaaagaatatagagaaaagatgctttgagcactttaggagaaaatttccctgaagcattttgaaataaattactcaattatcatgttttaattaagttctgctagagttgtggggaaaagtgaagaaggccaccagagatgaaggaaagtgacaaatagcaagaggagcgaagagatccttggaattcttgatcttaccaattgtcctctggcaacttccaaagagccacataaggaaaggctgctagtttctattaagcctagaagagaccaattattcttctactgtatatacagaaaaatagtccattctgtacccagaaaaaaatggaggggagaatagtgaaaggaaactagaacctaattatgaaatatttaattgggctaatatttgcctataccatattacctaacttgtttcattaagatcaaggataagtagatttttaatatcctttcagggctagaatactgaaaatcttactcattttgtatgaatttttttctcttgctatttcgatctgacttattgatgatgttttagattagtcaccttagagcaatttccctagattcctttaaccctgcatatcagtaattctgtagaacagaaaagaatattattctacttgcaaggaaaagaggaacattaaaccttgtgaaatgttgaaaaacatccccgattactgtaatctgcagggaaatcagaatctattatgagaacagaagagatcattaaaaataattgggaagattgtaaaattaaagattttacctttttctttactctgtccaatcatttttctctaaactttttcatatctttatgaccatccagtccatcggacatcatgggtgatcaaaactgcttaatcctgaagtaatgattactctttaactatcctgggatttcatagtataatagaaaaagcagtacctttttttgaaccaaaaaaaggggagttgttttatgaaatgtttgtcagaatcaagattttctaaaggactagacatgttctctaaggaaggttaaagaaatcatttcattctttcactatatgctagaataaaaagggattacaaaagccaactactttccctctgaaattctaagtgtttcagatgccctacattcaagactgccaagggattgccaggaagttacattattgctaagtcaaggaattttcttgaggaacagaaaaaattaattggcaccattttcatggaaacttaacagatttcttcaactcaattttcacatcctgaaaaaatatagggaaaatggtgaggactttggatcacagataaaagtttccatcaaaatacagttataaaatctgttttcctgagttggttttgaacttggaaaatgccaaagtggtattgcagaatatgctgctagcacatctctcccaccccttcccaaaactgagcttcacagggtggtagagggtttgttcatcaacactgcaagccagagaactgaattttgtcagtagagtttggagttgatcagagttttgaagtctgagtcatgttaatgatgctcatagaggactgatttgtaacccttaatttcagtctattaagtctatactagaggaagattccttcccagttactagaatgcttggattcattagacccctgacaaataataaattttcttttttttttcaaataataaattttctatgaaatattattagtaattagaagaccactaagtgaacatgatcaagtcaaagagggctggaggaacgtaagggctggttcttcaaagaaatggaaatatctggttaattttccctgccttatatgttcatataatagtcttggttcaagactactcatatctcccatactcattacacttttttccctctctgttatatctttttcttggactatgtcaaggggccttttctatcctcactggaatcatctgcatatcattaacatagtcttacctcaaatatgatacatggcaactgatgaggtttagcattcctaccattctgaacttccctgtgattatgggtcacatccacaaatctcagtcacacaattaagtaacgaatctcatcaaggatgtgctgctgctttcactcttggaagataaaccgtttctttggggttaggtgggaggtaaggggagcacattcaccctagccggctgcctatattagtgactatattagtgactcccaaccctagatgagtataagaattatttgtggagcttctaaaatttggttgcctaagtcctaccttagacatgactaaaaatggaacacatcagaatattaatgagccataaactcaaaaccagcatgcaaaagacaaaatataaccaagagtaacttttttgaaggccatattgtgtcaaatttgactgctaactatgttttatttcaaaaatagtctatacaatataattttattttttgccatgttttaaccatgcttaaatccatgtttcacataaaaattcaaatctccaaccactccttattatattatggtatcatttgctacaaatgacttctgccgttatcatttgttaacgtgttttccatgttgttttcttacatctgaggcatattcccccaaactcataattctacaaaaggggaaaaaatagaagatatatcaagggaatcatgtcaataaaaacatgaaggaaacatttatttttgataatggaaactattcacgtgggtttaatatgcagccactaaatcttctctgctaagtaactatctacctggccttaagtatagtctctgaccactgactcttcaatacagactgaaaagtcattgttggctggtcttttcagagggtatgaatgctcaggggaaagatacagtcagggaaaatatcttgtttgtgttctccaagtgcctagtgctgcagaagtctgataaatgttaaacaacagaattttctctaagttggaggcccggagaaatgtagacatagatggtatcacccttacatagaggaaaatagaatgcttgtctctgaagtattttcatcaggctgtttttgctagcagtagatatattaaagagaaaagaggggggattatcaaaatgtggttatttgaactccataaaagaataatggcctcagttctctttttcagtataacttacatttccatgttcacaaaagtcttttcattaaagtcatacctgggaaaacttgtgtgataactgagttcttgtctgaaatggtgatgtgtgaacagcccatagtagaaagtaaagaggagggatttaatgttacactttgcctcctgagacactcccaataaagctctgtgatggagggatggtatgctcaagaagaattctgttagtaattgaggtggggagttcgatgcctacagaccaatcagtatatagcgccaaggaatcatgttttacctgtatgtggcaaagctgcagtaacaaaacctgctaacctccaggatcctcagctgtgaatttcaactatcaaagtcctctacaacctagagcagggaacaaaatttgactgagcacaaggaatagaagcaaaggataaatagacactaaatctcttggtgaacatgattctacagatgtgttccctttcaagaaggattgaggaagagtacaaataaataatatgagaacttcaaaaaccactccagagaaaaggaaggataatttgatcatgaagactcaggataattatacttttgtaaatgatttgtttaatgagtaagattgtcaaacaccaagtgatcaaaatataggtaatgggaaatacattcacacatacaataagaacatttcaactaacaatttctaactttttaaatcttaggcttttaaaagtattttacctgttttaaaaatattgtaaggcagtaaagaagtatagattattttgaattttcatttattacatttaagctgaactattcaatcttcaggtacagcacctgacacctatacaaaagccaaaaaatagaatttcagaaaactgtgaactcataaacaaattgaaaacatagggaagaaacagcagcagaaattctggggtcaatgtcaggctaagatgaaagcaaaatacatgaaagacattttaggtaagagggtaggatgataggaaacatatgacatgaagaaagagatctagtgacaaaaggaagcacgataaaaataaaataaacttaagctaataattcagtagcaaatgaaaatccaccttgatcacaacaaaaaggagtgttggtggataaaaatcagttattgaagttgagaacataattggagaaggattttttctcaataccaggaaggactataaacaatcagatgaaatggagaagagagatccaactcatgtatataggtttggttaaggtttttttaataaaatataaatacaatcaatgacatagctaaacaaagctttggagaacaaggaagtgaagggctacatgggctccctatgacccagaaaacaatgaggaactatactcacctttatttttttttaagattttattcatttactcatgagagacacagagagagaggcagacacaggcagagggagaagctctatgtagggagacttaatccctggaccccaggatcacaccatgagccaaaggcagacactcaacaactagccacccaggcatctcagatttttctattttaaaaagaatcaaaatcacttttaaaaattagagaatctgtgtaacaaaaatttgaggtaatgaactaaacaatttattaggaaaagatcgcaacttaagaaatttatttagtctggcttttcattcatgtaagaagaaaacagatacagtttttggataccccgggttttagaaaagatgtaatttctgtgtacatcatgaaagtattacccaaaatatattctaagtaaatgagagactaaacaaagtaaaatgctcaaagagaacttatggtattaaagaatttgtggtgaaaaatgaattcagtaaaacatggaattgcatttaagtaattattgtaaatttatttataaaatgtgattgtgaaatattgatgagaaaaataggtaatacagcaaatcataattttattgcaatattctgggtctgatgctcaaataatataaaatcctttaagaattagaagaaaatgtgaaattaaaattgtttcctttcactcatgacttagtaataaaaatatatatatataaggtaaacagtttattttataataaagtatataaactttttatataaaaatatatattaatttataattaaaattaaaaatttaaatttaaatttaaatatataattaaaaattatatatatttatatatataaagtatataaactttttatatttatataaataaagtaataaactctgaagtttattttataataaaatatgtaccttccaaatcacttaatattatatttatatgcgtgtatgtgttcatatgtatacatatacttgtatagtctgtataacaagtcagaaactagtatttttagcacatggaaaattattaagagaaaacatgaggggtaggagggattcgggctaagctggcctaacagtattcctgctgaaggcagagccaggtgatcaaatatcacttaggggttgatggaaggtaaaggaacccaagcagatactgaaggtcatcagatatggagggtgggggagtcagagtacattaatgtagcaggattcttaggaaaattgaacaatacaaagacagacctggaagtacaaacagtgaagcataattgggaagaggactcagagaggccgactcaagttaggtcaggggaggaactctggcaatacaatatacagccttaagtttaagaaatggggatactttgtttggaatttacatataattcagtattccaggtatctcctaattcactcacatagcccagagttaattttcctcctagttccttagttatggaacaaaaaaataacttcagacgtaaatcttggccactgggtttttaagttctgttactggcaagtcctttccaattacaaaataatttggtgagatggtacagcatggtatgacattggtacagcatatctggcatggcttggaatggtatgtattccatgggaaagcctattagcaaagtgggagaaagtttcttactgtaaaaacaatatagggtcacctggatggcccagtcagttaagcgtctgcctttaggtcaggtcatgatctcctgggatccagccccacatctagctccctgctcagcagggagtctgcttctctctccctctgcccctccccctgctccttctctctctctctctctctatcaagtaataaaatcttttaaaaaatgtttttaaatacagtattatcacagaaagctgataattagaaaaaagtaatctgatactatcacaatagaacaacttatatcatcttaagataatttccctccatgctttattttatgaacatttatatcattaaaattttactatgtatacaaatttgtattcttcttctgtactcatcattatgtaagtacttcttatgttaccacatggtcttttctttcacatggtcttttaattattgtgtttattttaatggctacttaatatttattgagtaaatatttcataaagtaaattttgttgtttagcaatcaagttgtttcctttcaaattttcagtaaaaaatgctcttccagccagtcactcagtaatagaaataacttcatgaaaagctcggttcactttcatatctcatctgaagattcatacactcaccacaaaggtgttacttgatgttatgtaaccaatatttgatcaaatagaaaatcttgtaacgagaaactgatccaaaatagtaatagaagtaattaaaactctagaaactatagtttgtcttaaagcttttacattaagagaccaagagtctggaatatgtacagttagcaaactgacgaatgcttttcaacaaaaatcctgttaaaatcatactaaatttcctgaaaataaactaataataaatataaatttggggaggaaagaaaaaaactttccatcccacataaaacagtaatctaaatgtattacagcaattttgcacaccctacaaaaaataacaggttgcacgttttgggttgactagtctaccctcaggagaaaagataataaaatgcctaaatcattttgttttgcaacttgtcatggctaatattaatttatcaagacaattgcttcaaactttttagtgagctgggtggtgggtgagtaaaagcagaagtatgaaaaaaaaaaataaaaaaataaaaaaataaaaaataaaaagcagaagtatgtaaacttagtctaattgtaaacagctcacttccagaacttgctgtctagaaaacccccgctgctcctaacttttgttcttttgttctctctgcacaaccacgcaagacggagctccaccccctcaaaaaaagtcatcttacttatattttagtggtgagaatttaaaagatgaagggataattgaaatgaatactgttaagatgattcagtatttttcttgaattcagctaaacctgcacatactgcaaaacaagctgatatggttttccgggaactttccttgagacgacttcacacaaaaacagaaaatccagcttcataggttacttgaaataaaaaacgcattcatccttagtcaaaggaatgatacaagagagttgttagttaagttttagctattaaatgatgaagctaaacctccatactctggcaggaatggcccgtagaattcttttgctaactgcacctcccaggttggagtagggtagggaggcctataaaaagcaaatggtataagaagcaagatctcaattgttatctgttttttggctgctgaaccagtgagccctctggctgacttttgcttctccaattctcctgggatttcatgctccgtcatccctgaagaaaaagatttagagtgaggaatctgttgacatcttgttagctctggagaatagaacttctgaagagaaaagacaagccaagaaaacccccataggaatcggaatgattggccatggagcaattttatgtgtatgtgaatatttttagaaaggaaccctctctctgtccctgttccaagattacctgctagaatccttctcaaaaccaagaatgccattaaaggtcattccaagacagccccaaggctcaagtcccctggaaatacttacaacctgctatgaagtacttgaaatgcttcttttccagactaaccaatctgaacattatcaatgaatccttattactgtaaaatttgaggggcacctgggtggctcagtcagcatccggctcttgatttcagctcaggccataatctcagctcatgtcatgatcttagggtcatgggatcaaactctatattgagatctccctctctcactgtccctccccccactacgcatatgctgtttgtgtgtgtgtctgtctgtctgcctctcaaataaataaataaatattttttaaaatgtgaatgccctccatttcctaaattcttatctgtctgatctagtgacagtctaattgatctgtttctctcttgaagatgggttcccagtagtgagaaattctttttttttttttttttttttttaaagattttattttttaatccttatttatttatgatagtcacagagagagagagagaggcagagacataggcagagggagaagcaggctccatgcaccgggagcccgacgtgggattcgatcccgggtctccaggatcgcgccctgggccaaaggcaggcgccaaaccgctgcgccacccagggatcccgagaaattcttttttttaaaattttttattggagttcaatttgccaacatatagcataacatccagtgttcattctgccaagtgccccctcactgcccatcacccagtcaccccaaccccccacccacctccccttccactaccccttgttcatttcccagagttaggtatctttcatgttttgtcaccctcactgatatttgcactcattttctctcctttccctttattccctttcactaatttttatattccccaaatgaatgagaccatataatgtttgtccttctccgattgacttatttcactcagcataataccctccggttccatccacgtcgaagcaaatggtgggtatttgtcgtttctaatggctgaggaatattccattgtatacataaaccacatcttctttatccattcatctttcgatggacaccgaggctccttccacagtttggctattgtggacattgttgctagaaacatcgtggtgcaggtgtcccagcatttcattgcatctgtatctttggggtaaatccccagcagtgcaattgctgggtcgtagggcagatctacttttaactctttgaggaacctccacacagttttccagagtggctgcaccagttcacattctcaccaacagtgcaagagggttcccctttctccacatcctctccaacatttgtggtttcctgtcttgttaatgttccccattctcactggtgtgaggtggtatctcgttgtggttttgatttgtatttcccagagtagtgagcagttcttcagggacagcaaaccagtacacagctggacaggaatacctattctctcattctagactctgttgacatatttagatttgatactgcatgaggcatatactgctgaggagttacccaatccctcttgagataataaaataaatatgttgtacttattaaagaaggtacaaactcagatgagttctgcaaccaggcagttaacaagtgtgatgtcatctggtgaccataaggagtgacagggattgaggtagtctggagagcacagagcatccttaagggtatgcagccttttttatgtctttaaacactacattggacaaataaagcactgaattcacccatggtctgtggtttgctgctactgtaccttggactggtctatggtgtggcattagctgcagcacatgggaaattggaccatccaatgttcttattgcgctgagaaaaataaactcttttggaaagagaatagtttgttacaaaatgagcagcaattgcttattttaaatcttttaggagtaaagttgtcaatgttcctaaaaatctcatgatacttggatgttggttaggatctctcttcccaaagctacagagattatgtttattccacaaatgtttatcattgataataatgttaatctttatttaacttactgcatgacaggcactatgctaagtattttgtgcatgtgataaaaaataatcctcacaaaaagaacatgaaatttttctactgcccacgactagtacaatgcagaaatttactactgttctttctctgtttgtccctttttctttcctcagtgatgacacacattctttatgcccataacactaggtactcggcacaataatctttagaaaaaactcattaaaaattttgtactgatttattctgtaactgcttatagactggcaggaacaagaaactcaactgattaatttttacagtgcttttgttccccagttgttatatattgttttcttgttaaaacttctctcctcacattgtagacagagctcggatggagagtcaagccttttccaccgtggactgccttgctgctggacaggaggaactcatcctcttgcaggagaaggtcaaaaatgggaaactatctgtggatgaagccctagagaaatttaaacattggcagatgggaaagaccgagctagagatgattcaacaggtaatactgaccccaatcccttctgcaacattcactcacttgcagagcctctctgtaaatgtctttacaagaaacacaggtaactgttcttttgattttaactgaccacaaaaaagatgtttttcaaactaccaattaaatgattcatgtttataaacgatcaatgaactcagtgctgggacccagtgcatcagagataattcctaatcaccttataaggctgttcttttcttttaagattgtatttatttattcatgagagagagagagagaggcagagggagaagcaggctcctcaagggaagcctgatgcaggactcgatcctaggagcctgggaccacgacctgaaacaaaagcagatgctcaaccactgagccaccaaggtgctcccttataaggctgttcttaacttagtaaatgttttgtgaatctcattgctgaaattcagatgaagcatctttactctaacatgtgttcagcgttctgaagagtgtattatgttaactaggcttaaagggaaaaagaaaataatttaaatcatattaggtaatagtcctcaattagatatattaagttttctcttctcttccctttaacctttgggggaaaaaaactatatatatcagtcaacatttactgagaaatgctgccaagagtattaacttcaggtgagcttgtattctattaatataacatacatcccaagaggtattaggtggttcttctatgaacatgaaaacaaaatatgccatatccaccctatgacattcctcagagaggggtatcaagactggattccgtgtcatcccacaagccctcctactcccaaaaacagttttttattctcatcacagaaagccccagcgccatagaaaccagttcacaggaaactgattaaacacatttaattcgctagtttagaccaaagtacaaataatcattaatagcttaatatactacctttcccaggagatcatttttattgaaaaacaatgcttaggaaaacatttttaaacaacagaaaaatgagtcccagtgatagcatttcaaacaagtggagcttataggcagaaggtagtttaggcaaggggacttgcccagcacctctgagatcactccttctcagaagccacttgtcttgaaggacagttctggttggcggtggctggtcctgagccaggttttgcagtgccaagcaaggccagttgttttgccaaacaaatgggaaagagtgctgattttacagttctacaactaatcgtcattcctgatcagaatgatagccttgcttctcaacctgcaccaaaaccatcactgtcaaaggtccctcttaccctaaaaggataaaatgtgcctaaatgtagacctacacaaacactttcctagcattgctagcaaattttgttgaaataagtcaatcatacttcattttccatggaccataataaggcttacaatttagagaatccaaccgaaataccttcctggaaattattttgctagttgttattttaaagttttggttgtttttattttgttgtatacttatatttatttttattttttaaaagattttatttatttactcatgatagagagagagagaaagagagagagatagaggcagagacacaggcagagggagaagcaggctccatgcagggagcccgacatgggactcgatcttgggtctccaggatcaggccctgggctaaaggcggcactaaactgctgagccaccagggctgcctctgtatatttatatttaatattgtttttgaaagaatttagggtggcctattactactttgagagtggtctgacttgtcgtgtatatagggataatggggtaatgaatacgtgtagtgtttatgcaaaacaaaatttacctgtaaatacaagaacaagcactaagactgctttccattagagccagagttcagtgactctgaatgaatacttttttttttttgaatgaatactctgacgatcagtcagtctttttcctttttatgtttccagctttagtaagatataattggctataacattgggtaaatttaaggcataccaccgtgctgatttcacacacttacatattgcaaaatgattaccaccatggtgttagctaacatctccattatgtcacaaaaggatcatttccgtttgcggtgacagtatttaatagtgtaacatttaatactctcttagcaactttcaagtatatcatacagtattgttaactatagtttccatgctgtgcattgggtaccagaacttatttctcttaaaactagaagtttgtaccctttgaccaacttctctttccctccactcccctggtccctggtaaccaccactctactctcttgagttcaggttttttagattccacatataaatgatatcatactgtatttatctttctcctctgacttttttcacctagtatgccttcaaggtccattcctgtttttcaaattgcaggatttccttcttttttaatccattcatccattgacagacacttaagttgtttccatatcttggctattgtgaataatcctacagtgaacatgaaggtgcagatatctcttcaatattgtttagttgtgttcagatatatatccaggagtgggattactaaatcatatggtagttctatttttaaatttttaagaagcctacatactgtttttcaatatggctgcaccgatttgcattcccaccaacagtgcaccaggattcccttttctccacattctgtcaaccattttaacaggtatgaggtgataataccaaaatgtggttttgatttgcatttccctatttattagtgatattaagcaccttttcatatacctgtcgaccatttgtatgtctttttttttttttttttttaaaaaccctatttatttcctctgccatttttttctctggccattttttaactatattatttgcattttgctattgggttgttgaattcttaatatattttggatattaaccccttaccagagatatgatttgcaaatattttttcccattcgttgggttcccttttcattattttttatttattttggtgtgtagcagctttttaggttgatgtaatcctacttgtttatttttgcttttgttgattgtgctttggggtgtcatatccagaaaactattaccaagaccagtgtcaaggagcttattcccctacatttttcccctatattgtgataacccacaataatagaaaagggaatgttttccacatttccccaacatatgtacacactcattcatgtacacttcaatattaatcttgtagtttgcacaaatatccatttataccccaaggataaatctatcagaggtaactgtgatattacattattgacagaggtattatctgaagaaaataagaggaaaaatggcacaaacatagagttcataatagaagagtttctagagtagtgaagcccatgtagaacccttggagttgaaggtgagatggttgagaagataggaacgttgactttcaccttcctcactgctgctcataaccactgagtgcctatagaagaatgtttaccccactcacagagaggatgtctatggcaatgaaaataactctctttttaagaagcatggtttctaggtttcaggtgaaacacttagggaaaagtgctattcttagattctcttctaaaagatttttgaatgttagcctgttttacattttggttaaaatgataatagatgttcaatgtagaatgttttagaaagcaaagaaagatcacaaatgagctaagaaggggagtccatcatttgtttccaagttcctagttcttagaaagcatttataggaaaaaatgtgcccacagaggctgaaagtaggcttaggcagtagcaaatcaccacagtgtcacactaatgctcttggacaccagatagccaagatagccccctcaggtgttagattgggaaaaaacagcaaccacaagaattggggtttgagtcccaatgctgactccatcacttacaactgtgtgttttgggaaaattatgtaacctctctgaatctcagtttctatatatgtaaaataatatggtgatatagatactttatgactttgcaaatattaagtaagataacatggttaaaatatcagggatggttgattgggacagtgtaagtaccaaattagtgttatacagtcatggtagtcctagtgacagtagttgaagttctcatttgatccatgacaaatttgtagatattcacttgtattttctggtcatttccttattaaggaaaaactacgccaactacgagattgcattattgggaaaaggccagaagaagaaaatgtttctggtaagtaagatttgcctgttattcctttatcactctaaaagaagaaaatgcaagggctgtggagtcaagcctgaattcaaatctgcgcttcatcacttcccagctgtatgcctggacagtgtctaatccttggtacttgaatcccttgtctgcaaaacaggagtagttatagatatatctgaaagtggttgtgtaccttgatatctaaaacatttttcaacttctttgatccataacaaaccttcaataaatgctaatttctttcctttgatttctgtgaaatttgcaaataatttaattccaccccataattttaatatgtatgagccattacacagagtactgaattattgagtcatacttttttaataaggttgaatattctcaatacatcagttctcacaattgtccacatgtggataattttaaattataaatctcaggttgttttcccatgcagcaagcatgtttcttggcaatgcagattccccttgctaataattaacaattgttaattgttctgggaggggggacactcgttttaatcttaatacaaaccaaacattattaagttggtaaatcttctccaaagataaacagcacattccaaaattgaaaataaattatgttgtcactattggaccactctttccctaagtccttaacccctgcacagataattaaaaactgacaaccactagcaaaccttgaaatttccatctcttccattttcagtaagattgcataggttttagctagtgcttcatgttgaaatttaccttgaaaatttttattttcatattattaaattggtatctgcttttccatagaaaattctttaagctctcttagaaatgttttctaaaaacagaaatatactttctaaaataattaagttaataaacacggggctgatttacagatgactgagtcctatagatctccattctaatgtggaaaaagattaaaattttaacaatgtataaaatcacaaaatatataaatacttttactaaggactattaggtagtaacagctaatatttgactactatacacttaatattaccttttgaatttcttacaataaccctttgaggtagacattagtctgaatttataaataaggaaactgagattgtaagaaactaagaagcccaaagacacacagactagaaactacaacttatttttttttaggtgggcacttgacgggatgagcactgggtgttattctgtatgttgacaaattgaacaccaataaaaaaaaaaattatttactccaaagtcaatgcttttaatcattgctgtcttaattaggaatcagctagttccctttctaaccaaaagtagtaccagacaaaccctcccactgtacagaacactaacactgggcaaaaatatgaggcagttgacttcagagagtaggcagttaacagcctaagaccataattccagtgacaaaacttacagggtggctctaatgatgacccaggagactgattttctgactagagtctgtgcaaagcaaggcaatcctttgaaatgaggagggaaagatcagattcagagcaaccaaagcttctaagaatgtgtgtggtgggtcactgaagaggagatggctgtatagagaggtagcctcagatggagtgagggatttcacaaatctgtggtcaagaggtgagctgaacatgtgcagagcaagaccactgaggcatataagataacagcttctaggacactgaaagcaaaacagatatactagagtttgagcagagccagggaaaagccatactggggaagtcgagttcttgccctgccagagtggaaagatcttcttaacaatttgttaaaacattgcatcctgctgtgacacccaagaaatgtaatgattaaagaccatgacttagggtgagaactcctgtagacctgccctaacaaagcctaaaaccaagcccttacaagagccacatggaagggagagtttgcaggctcattcctactgagggcttcagaaaattccttgggctatctataagtcaacattgaaaagaaagcataaaaccaggcctatataagttcatgccaataatcaaacttcctgctgaaataagaacctacacttattagtagaggaatattacagaatctagaatatacaattgattgtctataatgtccagtattcaattttttttaaaaaaggaaggcatacaaagatataggaatatgtaattcatagaaaagaaaaggaaagccattaataaaaactgaatctaaaatggtcccattgtaggtcttaatagacaaatactttaaagaaacttctataaccatagttaaattaatttttaaaatatggttttaaggagagaataaataggaaatatattgatgtttacagaacatgacatgggaaaaactaagagtacatatttgcctatattctcttaccacaacagaaagaaattatgtttttttaaagatttttcttacgagagacacagagagagaagcagagacagaggcagagggagaagcaggcctgtgcaaggagcctgatgtgggactcgatcccggatcccagaatcacaccctgagccaaaggcagacgctcaaccactgagccaccgaggcgtcccagaaattagaatttgattgaaaaattagaaatgtccctccaaatcattaggaattaagtacatttcaaaataacccatggatcaaataagaaatcacaagaaaatatttcaacctgaatgataatgaaagtgcaacatatcaaaatgtgtgaaatgcaactaaaatagtactttgaggtaaatctttggctataaatattgtagtagaaaaaggaaaagcttaagatcagtgatctaggtttccacttcaggaaactggaaaatgaaaagcaaactcaaaccaaaaatgaggtaagaggaaaaattataaagaacagatcaaaagtcaatacaaaatgaaatcaaaaatcaatatcaaattaaagagataaaaccaaaaatcagttatttgaaaacataactaaaattgataaaatgaactggaaaaaagcaaaaacaagagttaccatatcaggaaagtaaagtcaggttatcataacagatcctatagatactataaagatactaaggaaaaataattaattttttgccaatacacttaacttagacaaaaagaataaattaatcaaaaagcaaaacttacaaaactaaaataattacactcctttgaatgaaatgttttacatatttaacaaagatgaggctgggaaggaggcaggaagagcagaagaaaatgaacctagaggtgttaccatactgaaggtagacagaagaaaactaacaaaataatttagaaaacagaatattccaaagataaaattttttaaaaaatcagtacacaaaaatgtattctgtactccagttagtaggtttatttctctatagagttttagctcagcaatttcaaaagtaccatttgtacattcaagatggagtaaataaataaatgtaatatggatagtaatagccagacttcctattgtcagagaaaggagtaacttctaaggaaagaaggaaggttggaatgaatcttgtggttctagattggaattggaggtaaaagtaacaacttattacaaatagatatagaaatataactgtgtgtataaaaatgtgtttctttagcccttctgctgaaaagccctagaagcaaggacagcccagcaccaatgagcacatctcatacctagaccttagtttataaaaacaaatggcaaattatcatgtgaaaatatgctcaacatcattaatcatcaggaaaatgcaaattaaactaaatgaaataccaatacacaacaaatataatggctagaatatttagatattgtcaaaatgaagtgataaggatatagtacactggatatgcattgcatgaattgctaatggaagggtaaagtggtgtcatgaaacagttgtgatgaaacagttaaatgtgcacctaccttatggcctagacaaaccattcctatgtattttgccaaaagaaaaaaaaaagagaaaagaaaagaaaaggaaacattatgtgtacccaaagtcttgttcacaaaattcatagcagtttcatttataatggccaaaccagaaacaacccaaatgtccaataatagaagagatagttataccatagtatattcacacaatggaatatagtcaacaatagaaagaaatacctactatgacacacaacaacacgaatgaatctcacagctttatgctaaatgaaaaatatcaaacacgaaagagtacatggtttgagattccatttatctggaattctagataaaaactaatctatagccataaaaatcagattggggtcactaggggtaatggtggagtgaatattaatgggtggcacaagggaattttctgtagaaatgaaatgttctctctcctaactgggtgatgggtacatggctgtatagatttgtcaaaagtcgtaaacgtacatactttaaaatgtctgaattgtactaaacatactgaataatgttaatttgaaaaaaagaatggaatatcacaggctaaaatatattaaagcagaatagcaagcttttattaagtgtctaatgtttctaaaatcataacaccttgggacaaacatcaggaggaaaaaagacaaaagtagtaaaagacatgatttcacactttgtggccatacattagtatctttgtaaccagactattagatttcatagtcacttcaaagacaagaagtagtcaaacaataaaaagaagaaagtttaagataaatatagagcaaaactcaatagaatgtgctttttctgtcttctagataaactcactattgtgcaccatccaaatggtaagtttttgctatttaccaaagaattttggactacaacaaagtatatgataaaccagtttcaaaattaaagtaaacttaatatgatgaggcaggtcagtgcaaaaactcatatagattttcatatgtatatgcaaaaccacttgacatccaaaataactttgaggtgattcgagaacagcaatgtagaggtgtggttacacagtaaaagaagcatgtgctttggactccaatgggcctagcccttcacaacttaccagttactacctttgtcaaaccacttaagctgtttaggcctcagttgccttgtatgtaaatgtagacaagacttctacataggggattgctataaagactgataatgatgtgtcaatgggagtcgatgcttggtaagtgggaattattgataaagcaaatgaaagtggaaaaacacttgagatggtgattcactttagtgtggtggggaggaatgtacgcagaaggttgggagggtcaagaggagccagaggccagctggggagccctgactaaacttggagcaactgtctccaacctgaaccaaagcagtgactgtgagaatggagaaaagcaatagaacctagccacatggatgggattgaaaatggaaagcctgtagtaaaggcaggaggagcagtggccacaccccttgtttcttcaataacttttccctttacccatatcgtactcaaagcatgtaatgttttttaaaagtcctgaatatatgaatcattaaatacgcgaaagactgaagaaatagtaacagagtttatatttctaggtaatgaaagtgcccgcaatgaaaatatgttacataacatacccttccgcaataaggtaggttgtatttatgttaatttttataaaaatgcaaaaaaagaacttctttgtcgaaaaagaatgtaagagccttctatcctgactatgctttgaaacattttaacaactatgaacaaaataggttacataagcgtttgggtgcaaattactgcatgagcagtaatgtaaggaaattaactatttaggaaaaaagcaaagaacccaaagggaaaagtcaccttggccaaagtatgactgaacttaacatgtacagatcaaataaatcttagaggagttttgcatatatcttttttagcaacaggaatatgatgagtctatttgaatgacagtgtacacagtccttaaagtctagctgtaggaaaagatgtatccccaaaatatcattcacgtaaatatacatcacttcacatgtagagctaactgaaaatgaattgccagcaagaagctaaaaataacactaagagagcagcaattgaaaaccaagactaacttaatgagatgagcactgggtgttatactatatattagcaaattgagtttaagtaaaatcttaaaaaaaataaaaataaaagaaaaccaagactagttcaaggcaacactgataatgtccagaccataaattcttcctcaaaaacaggcatgttattccaactatctacatatgtgatgtaggaaattttcagtattgcctaggatcatgtggccctagatttcttgctaaaatctcataatggtaaatctcataatggtaaattctcataataaaccaattttaataacgcttttaagttatcttatgttttaggtgaatcataaggaatcataaaagtcatttacccttaaggactgattgaaatatttctggatcatcctgtgacccaccagtaagctacagacctagtgagtctacagtatatggaccctttagactcactggaccatagcagaatcatggggtaccacctaggcctctgagtttatgaaaatacctttgtcaaacaaagatccttctacaagatctggaggactatgcaaccatagcatactttttgagaacaacatttggggtacgaggaaaagagtaaattcccaacacttaggctattctccatcaagatggaaataaaatttgaactcaatgactttgtgtaggtgagctttacaataattgtttccttttaagatggaggaacgtcttgccaattccctcaatgaagctgttgtcctataaaaggcaactatgactttatcatgtttctttgttctgccttaaaatggagaaatgctagctctgtatttttttttccttagcttcctactcgactccaaggtgaaaaggaattcagtttctgttgcaggaaagattattaaagaaggtaaaatatcagctatattttttataaagtttaaagcaggcttattagagacttgaagggaccatttgtctagcacacagaatactttaaatatttcactgtctcctcctgaaatgataaacatatgtatttttcaaggtaaaggtgattgttccttagaaagatttatgtataaaggtgattttatgctagctaatcaatttcgattgacgatccattattcactaatctatttcctgtgatctcactttcaggttattataatgaaacccaaaaatctgcagacactctgctttctgagcgaagcaagcttgcatttggattgcctactatcttcagggaaaataccatgaaagcagaagtaaacctctggatttcacaatttgttattgccacaatttaacttgcttcagataggtatatcaagattccaattgaatgccatagagcagttgcattctttgacaattcttggagttttcagtgcattttgccactgaagcagctgctacccattttgaaaattgaagaaaaacaagagcagagaagtgaaatgctctgtagcaaagttattgcacctgtttgaagcactaaccttaacattttactcctctgtcctcagaatgaccatggaaatacaaatttgttcaatacatatttgccttttaagtggctggtcctttttctgaatttctgacattcaaaggtctaactattattgtcacatcatgttttcagaaaccttcctttagttagtgcccattattgagagaatattattttaactttctatatttcaaattaacatacatttctacaacctacattctttattaaaaggcttacaattccagcagcctatgtcttagaaactatgtactagcttagaatagaacacagttttattgttctatgatttccatgcctgattcctgcaaataaaatgaatgtttaaaactatgtctgactttccaaaataaatgtataataattaaccaccaatttacagaaggaagcctcctgaaaaagcttttaattcattgaatttcattgcataagccccacacagtaaatacacaaatgatttttttctagcttctaaacagtgttaggaatatctatttataatgattctgcaaaatattgaccacagtttctcatgactagtaacattctcaaataaactcaagttttaaaaaataatacagatgtttgattcatccctaacagatgatgctttgattgtgacatctggaaatctagtccatagtttcacacctaagctttcgagaaacagcatgcccttccacacactgagcccccatttatgtggactgtcccacccaaagagttcagaaaagcactagaacaactgactggttttatataattttctgcatctctaaattcaaatatatgtattgtctctaaagtaaaaacctctatattggcctgtgactgatacttacagtttcgtcattggctttattcttattaaaattgtgaaatctagtgacaggtgaaatgtcaacttttctaattataaagcaattggattatctctaagctgagactcaattaagagtggaagagaaagatgaggcttgaatattattcaggtctttagcatagatggaaatctaaaatcagggcttctaaatactacccttctgaactgagctaacttagaataaaaccatggcaaaaagcccctttaatagtctctttcttgggatactctggccctcgaaatcttaccactttgtcatcacaaagagacctggtaaaaaggaaacctaaaattaactgaggtcttcctcatgcttcctctaccatgactcctcccaac

(19) United States
(12) Patent Application Publication
(10) Pub. No.: US 2016/0060699 A1
(43) Pub. Date: Mar. 3, 2016

(54) SLE AND SLE-RELATED DISEASE-ASSOCIATED RISK MARKERS AND USES THEREOF

(71) Applicant: THE BROAD INSTITUTE, INC., Cambridge, MA (US)

(72) Inventors: Göran Andersson, Uppsala (SE); Helene Hansson-Hamlin, Knivsta (SE); Sergey Kozyrev, Uppsala (SE); Kerstin Lindblad-Toh, Malden, MA (US); Maria Wilbe, Uppsala (SE)

(73) Assignee: The Broad Institute, Inc., Cambridge, MA (US)

(21) Appl. No.: 14/783,470

(22) PCT Filed: Apr. 11, 2014
(86) PCT No.: PCT/US2014/033791

§ 371 Date: Oct. 9, 2015

Related U.S. Application Data
(60) Provisional application No. 61/810,794, filed on Apr. 11, 2013.

(51) Int. Cl. C12Q 1/68

(52) U.S. Cl. CPC C12Q 1/6883; C12Q 2600/156; C12Q 2600/172; C12Q 2600/112

(57) ABSTRACT

Provided herein are methods and compositions for identifying subjects, including canine subjects, as having an elevated risk of developing systemic lupus erythematosus (SLE) or an SLE-related immune-mediated rheumatic disorder or having undiagnosed SLE or an SLE-related immune-mediated rheumatic disorder. These subjects are identified based on the presence of gem-line risk markers.

### CROSS-REFERENCE TO RELATED APPLICATIONS

This application claims the benefit of the filing date of U.S. Provisional Application No. 61/810,794, filed Apr. 11, 2013, the entire contents of which are incorporated by reference herein.

### FEDERALLY SPONSORED RESEARCH OR DEVELOPMENT

This invention was made with U.S. Government support under U54HG003067 awarded by the National Institutes of Health. The U.S. Government has certain rights in the invention. The research was also generously supported and funded by the Swedish Government and Uppsala University.

### BACKGROUND OF INVENTION

Systemic lupus erythematosus (SLE) is a chronic autoimmune disorder. SLE tends to be clinically heterogenous, with manifestations ranging from relatively mild symptoms such as skin rash to severe impairment of functions of kidney, heart, lung, central nervous system and other organs. While SLE and SLE-related diseases were first described in human patients, they are also seen in other species including dogs with similar clinical manifestations. The most common clinical signs shown in dogs are polyarthritis, fever, anemia, skin problems, and rarely renal failure.

### SUMMARY OF INVENTION

The invention is premised in part on the identification of germ-line risk markers (e.g., SNPs) that can be used singly or together (e.g., forming a haplotype) to predict elevated risk of an SLE or an SLE-related disease such as immune-mediated rheumatic disease (IMRD) in subjects, e.g., canine subjects. The invention is also premised in part on the identification of particular genes that when up- or down-regulated that can be used singly or together to predict elevated risk of SLE or an SLE-related disease such as IMRD in subjects, e.g., canine subjects.

As described herein, a genomic analysis was performed on DNA obtained from canines having different sub-types of IMRD. SNPs on chromosomes 11 and 32 were identified as being associated with IMRD, and highly associated with a sub-type of IMRD: antinuclear antibody (ANA) positive IMRD with a speckled nucleoplasmic staining pattern (also referred to herein as speckled ANA-positive IMRD). SNPs identified as associated with speckled ANA-positive IMRD were found to correlate with decreased expression of PTPN3 and increased expression of DDIT4L and BANK1, indicating that the expression levels of these genes may correlate with the presence of IMRD, such as speckled ANA-positive IMRD.

Accordingly, aspects of the invention provide methods for identifying subjects that are at elevated risk of developing SLE or an SLE-related disease such as IMRD or subjects having otherwise undiagnosed SLE or an SLE-related disease such as IMRD. Subjects are identified based on the presence of one or more germ-line risk markers shown to be associated with the presence of SLE or an SLE-related disease such as IMRD and/or expression levels of one or more genes shown to be associated with the presence of SLE or an SLE-related disease such as IMRD, in accordance with the invention. Prognostic, diagnostic, and theranostic methods utilizing one or more germ-line risk markers and/or expression levels of one or more genes are also provided by the invention.

In some aspects, the invention relates to a method, comprising:

a) analyzing genomic DNA from a canine subject for the presence of a single nucleotide polymorphism (SNP) selected from chr11:67543652, chr11:67538032, chr11:67516041, chr11:67537363, chr11:67538806, chr11:67537493, chr11:67536944, and chr11:67583604; and

b) identifying a canine subject having the SNP as a subject at elevated risk of developing IMRD or having undiagnosed IMRD. In some embodiments the canine subject is homozygous for the DLA haplotype DLA-BRB1*00601, DQA1*005011, and DQB1*02001. In some embodiments, the method comprises:

a) analyzing genomic DNA from a canine subject for the presence of a single nucleotide polymorphism (SNP) selected from chr11:67543652, chr11:67538032, chr11:67516041, chr11:67537363, chr11:67538806, chr11:67537493, chr11:67536944, and chr11:67583604;

b) analyzing the genomic DNA for the presence of a DLA haplotype DLA-BRB1*00601, DQA1*005011, and DQB1*02001; and

c) identifying a canine subject having the SNP and homozygous for the DLA haplotype as a subject at elevated risk of developing IMRD or having undiagnosed IMRD. In some embodiments, the SNP is a SNP at chromosome position chr11:67583604. In some embodiments, the genomic DNA is obtained from a bodily fluid or tissue sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay. In some embodiments, the SNP is two or more SNPs. In some embodiments, the SNP is three or more SNPs.

In other aspects, the invention relates to a method, comprising:

(a) analyzing genomic DNA from a canine subject for the presence of a risk haplotype selected from a risk haplotype having chromosome coordinates chr11:67536642-67583604; and

(b) identifying a canine subject having the risk haplotype as a subject at elevated risk of developing IMRD or having undiagnosed IMRD. In some embodiments, the canine subject is homozygous for the DLA haplotype DLA-BRB1*00601, DQA1*005011, and DQB1*02001. In some embodiments, the method comprises:

a) analyzing genomic DNA from a canine subject for the presence of a risk haplotype selected from a risk haplotype having chromosome coordinates chr11:67536642-67583604;

b) analyzing the genomic DNA for the presence of a DLA haplotype DLA-BRB1*00601, DQA1*005011, and DQB1*02001; and

c) identifying a canine subject having the risk haplotype and homozygous for the DLA haplotype as a subject at elevated risk of developing IMRD or having undiagnosed IMRD. In some embodiments, the presence of the risk haplotype is detected by analyzing the genomic DNA for the presence of a SNP located within the risk haplotype. In some embodiments, the SNP is two or more SNPs. In some embodiments, the SNP is three or more SNPs. In some embodiments, the SNP is selected from a SNP at chromosome position chr11:67543652, chr11:67538032, chr11:67516041, chr11:67537363, chr11:67538806, chr11:67537493, chr11:67536944, and chr11:67583604. In some embodiments, the genomic DNA is obtained from a bodily fluid or tissue sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay. In some embodiments, the risk haplotype is two risk haplotypes.

In another aspect, the invention relates to a method, comprising:

(a) analyzing genomic DNA from a canine subject for the presence of a mutation in a gene selected from PTPN3 and BANK1; and

(b) identifying a canine subject having the mutation as a subject at elevated risk of developing IMRD or having undiagnosed IMRD. In some embodiments, the canine subject is homozygous for the DLA haplotype DLA-BRB1*00601, DQA1*005011, and DQB1*02001. In some embodiments, the method comprises:

(a) analyzing genomic DNA from a canine subject for the presence of a mutation in a gene selected from PTPN3 and BANK1;

b) analyzing the genomic DNA for the presence of a DLA haplotype DLA-BRB1*00601, DQA1*005011, and DQB1*02001; and

(c) identifying a canine subject having the mutation and homozygous for the DLA haplotype as a subject at elevated risk of developing IMRD or having undiagnosed IMRD. In some embodiments, the gene is PTPN3. In some embodiments, the gene is BANK1. In some embodiments, the genomic DNA is obtained from a bodily fluid or tissue sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay. In some embodiments, the mutation is two or more mutations. In some embodiments, the mutation is three or more mutations. In some embodiments, the gene is two or more genes. In some embodiments, the gene is three or more genes.

In yet other aspects, the invention relates to a method, comprising:

(a) analyzing a sample from a canine subject for a level of PTPN3 and/or BANK1; and

(b) identifying a canine subject having a decreased level of PTPN3 and/or an elevated level of BANK1 compared to a control level as a subject at elevated risk of developing IMRD or having undiagnosed IMRD. In some embodiments, the IMRD is ANA-positive IMRD. In some embodiments, the IMRD is speckled ANA-positive IMRD. In some embodiments, the canine subject is a descendent of a Nova Scotia duck tolling retriever. In some embodiments, the canine subject is a Nova Scotia duck tolling retriever.

In other aspects, the invention relates to a method, comprising:

(a) analyzing genomic DNA in a sample from a subject for presence of a mutation in a gene selected from PTPN3, or an orthologue of such a gene, and, BANK1, or an orthologue of such a gene; and

(b) identifying a subject having the mutation as a subject at elevated risk of developing SLE or an SLE-related disease or having undiagnosed SLE or an SLE-related disease. In some embodiments, the subject is a human subject. In some embodiments, the subject is a canine subject. In some embodiments, the gene is PTPN3. In some embodiments, the gene is BANK1. In some embodiments, the genomic DNA is obtained from a bodily fluid or tissue sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay. In some embodiments, the gene is two or more genes. In some embodiments, the gene is three or more genes. In some embodiments, the mutation is two or more mutations. In some embodiments, the mutation is three or more mutations.

In other aspects, the invention relates to a method, comprising:

a) analyzing genomic DNA from a canine subject for the presence of the DLA haplotype DLA-BRB1*00601, DQA1*005011, and DQB1*02001; and

b) identifying a canine subject having the DLA haplotype as a subject at elevated risk of developing speckled ANA-positive IMRD or having undiagnosed speckled ANA-positive IMRD.

In some aspects, the invention relates to a method, comprising:

a) analyzing genomic DNA from a canine subject for the presence of a single nucleotide polymorphism (SNP) selected from i) one or more chromosome 11 SNPs and ii) one or more chromosome 32 SNPs; and

b) identifying a canine subject having the SNP as a subject at elevated risk of developing IMRD or having undiagnosed IMRD. In some embodiments, the canine subject is homozygous for the DLA haplotype DLA-BRB1*00601, DQA1*005011, and DQB1*02001. In some embodiments, the method comprises:

a) analyzing genomic DNA from a canine subject for the presence of a single nucleotide polymorphism (SNP) selected from i) one or more chromosome 11 SNPs and ii) one or more chromosome 32 SNPs;

b) analyzing the genomic DNA for the presence of a DLA haplotype DLA-BRB1*00601, DQA1*005011, and DQB1*02001; and

c) identifying a canine subject having the SNP and homozygous for the DLA haplotype as a subject at elevated risk of developing IMRD or having undiagnosed IMRD. In some embodiments, the SNP is selected from a SNP at chromosome position chr11:67536642, chr11:67535953, chr11:67543652, chr11:67538032, chr11:67516041, chr11:67537177, chr11:67537363, chr11:67538806, chr11:67537493, chr11:67485866, chr11:67504858, chr11:67518596, chr11:67518781, chr11:67536944, chr11:67537924, chr11:67511882, and chr11:67583604. In some embodiments, the SNP is a SNP at chromosome position chr11:67583604. In some embodiments, the SNP is selected from a SNP at chromosome position chr32:24556037, chr32:24667283, chr32:25537276, chr32:25392401, chr32:24606503, chr32:24650093, chr32:25798353, chr32:25485961, chr32:25007496, chr32:25007632, chr32:25642357, chr32:25485644, chr32:24672221, chr32:25702963, chr32:24618331, chr32:25049586, chr32:25779083, chr32:25484844, chr32:25816401, chr32:25718852, chr32:25305524, chr32:25710678, and chr32:25662984. In some embodiments, the SNP is selected from a SNP at chromosome position chr32:24556037, chr32:25485961, and chr32:25485644. In some embodiments, the SNP is a SNP at chromosome position chr32:24556037. In some embodiments, the SNP is two or more SNPs. In some embodiments, the SNP is three or more SNPs.

Other aspects of the invention relate to a method, comprising:

(a) analyzing genomic DNA from a canine subject for the presence of a risk haplotype selected from a risk haplotype having chromosome coordinates chr11:67536642-67583604 and a risk haplotype having chromosome coordinates chr32:24556037-25816401; and

(b) identifying a canine subject having the risk haplotype as a subject at elevated risk of developing IMRD or having undiagnosed IMRD. In some embodiments, the canine subject is homozygous for the DLA haplotype DLA-BRB1*00601, DQA1*005011, and DQB1*02001. In some embodiments, the method comprises:

a) analyzing genomic DNA from a canine subject for the presence of a risk haplotype selected from a risk haplotype having chromosome coordinates chr11:67536642-67583604 and a risk haplotype having chromosome coordinates chr32:24556037-25816401;

b) analyzing the genomic DNA for the presence of a DLA haplotype DLA-BRB1*00601, DQA1*005011, and DQB1*02001; and

c) identifying a canine subject having the risk haplotype and homozygous for the DLA haplotype as a subject at elevated risk of developing IMRD or having undiagnosed IMRD.

In some embodiments, the presence of the risk haplotype is detected by analyzing the genomic DNA for the presence of a SNP located within the risk haplotype. In some embodiments, the SNP is selected from a SNP at chromosome position chr11:67536642, chr11:67535953, chr11:67543652, chr11:67538032, chr11:67516041, chr11:67537177, chr11:67537363, chr11:67538806, chr11:67537493, chr11:67485866, chr11:67504858, chr11:67518596, chr11:67518781, chr11:67536944, chr11:67537924, chr11:67511882, chr11:67583604, chr32:24556037, chr32:24667283, chr32:25537276, chr32:25392401, chr32:24606503, chr32:24650093, chr32:25798353, chr32:25485961, chr32:25007496, chr32:25007632, chr32:25642357, chr32:25485644, chr32:24672221, chr32:25702963, chr32:24618331, chr32:25049586, chr32:25779083, chr32:25484844, chr32:25816401, chr32:25718852, chr32:25305524, chr32:25710678, and chr32:25662984. In some embodiments, the SNP is selected from a SNP at chromosome position chr11:67536642, chr11:67535953, chr11:67543652, chr11:67538032, chr11:67516041, chr11:67537177, chr11:67537363, chr11:67538806, chr11:67537493, chr11:67485866, chr11:67504858, chr11:67518596, chr11:67518781, chr11:67536944, chr11:67537924, chr11:67511882, chr11:67583604, chr32:24556037, chr32:25485961, and chr32:25485644. In some embodiments, the risk haplotype is two risk haplotypes. In some embodiments, the SNP is two or more SNPs. In some embodiments, the SNP is three or more SNPs.

In yet another aspect, the invention relates to a method, comprising:

(a) analyzing genomic DNA from a canine subject for the presence of a mutation in a gene selected from:

one or more genes located within a risk haplotype having chromosome coordinates chr11:67536642-67583604,

one or more genes located within a risk haplotype having chromosome coordinates chr32:24556037-25816401; and

(b) identifying a canine subject having the mutation as a subject at elevated risk of developing IMRD or having undiagnosed IMRD. In some embodiments, the canine subject is homozygous for the DLA haplotype DLA-BRB1*00601, DQA1*005011, and DQB1*02001. In some embodiments, the method comprises:

(a) analyzing genomic DNA from a canine subject for the presence of a mutation in a gene selected from:

one or more genes located within a risk haplotype having chromosome coordinates chr11:67536642-67583604,

one or more genes located within a risk haplotype having chromosome coordinates chr32:24556037-25816401;

b) analyzing the genomic DNA for the presence of a DLA haplotype DLA-BRB1*00601, DQA1*005011, and DQB1*02001; and

(c) identifying a canine subject having the mutation and homozygous for the DLA haplotype as a subject at elevated risk of developing IMRD or having undiagnosed IMRD.

In some embodiments, the gene is selected from PTPN3, DAPP1, LAMTOR3, DNAJB14, H2AFZ, DDIT4L, EMCN, and PPP3CA. In some embodiments, the gene is selected from PTPN3, DAPP1, LAMTOR3, DNAJB14, H2AFZ, DDIT4L, EMCN, BANK1, and PPP3CA. In some embodiments, the gene is selected from PTPN3, BANK1 and DDIT4L. In some embodiments, the gene is selected from PTPN3and DDIT4L. In some embodiments, the mutation is two or more mutations. In some embodiments, the mutation is three or more mutations. In some embodiments, the gene is two or more genes. In some embodiments, the gene is three or more genes.

In some embodiments of any of the methods described above, the genomic DNA is obtained from a bodily fluid or tissue sample of the subject. In some embodiments of any of the methods described above, the genomic DNA is analyzed using a single nucleotide polymorphism (SNP) array. In some embodiments of any of the methods described above, the genomic DNA is analyzed using a bead array. In some embodiments of any of the methods described above, the genomic DNA is analyzed using a nucleic acid sequencing assay.

Other aspects of the invention relate to a method, comprising:

(a) analyzing a sample from a canine subject for a level of PTPN3 and/or DDIT4L; and

(b) identifying a canine subject having a decreased level of PTPN3 and/or an elevated level of DDIT4L compared to a control level as a subject at elevated risk of developing IMRD or having undiagnosed IMRD. In some embodiments, the level is an mRNA level or a protein level. In some embodiments, the level is an mRNA level. In some embodiments, the level is a protein level.

In some embodiments of any of the methods described above, the IMRD is ANA-positive IMRD. In some embodiments of any of the methods described above, the IMRD is speckled ANA-positive IMRD.

In another aspect, the invention relates to a method, comprising:

a) analyzing genomic DNA from a canine subject for the presence of the DLA haplotype DLA-BRB1*00601, DQA1*005011, and DQB1*02001; and

b) identifying a canine subject having the DLA haplotype as a subject at elevated risk of developing speckled ANA-positive IMRD or having undiagnosed speckled ANA-positive IMRD.

In some embodiments of any of the methods described above, the canine subject is a descendent of a Nova Scotia duck tolling retriever. In some embodiments of any of the methods described above, the canine subject is a Nova Scotia duck tolling retriever.

Further aspects of the invention relate to a method, comprising:

(a) analyzing genomic DNA in a sample from a subject for presence of a mutation in a gene selected from:

one or more genes located within a risk haplotype having chromosome coordinates chr11:67536642-67583604 or an orthologue of such a gene,

one or more genes located within a risk haplotype having chromosome coordinates chr32:24556037-25816401 or an orthologue of such a gene; and

(b) identifying a subject having the mutation as a subject at elevated risk of developing SLE or an SLE-related disease or having undiagnosed SLE or an SLE-related disease. In some embodiments, the subject is a human subject. In some embodiments, the subject is a canine subject. In some embodiments, the gene is selected from PTPN3, DAPP1, LAMTOR3, DNAJB14, H2AFZ, DDIT4L, EMCN, and PPP3CA. In some embodiments, the gene is selected from PTPN3 and DDIT4L. In some embodiments, the genomic DNA is obtained from a bodily fluid or tissue sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay. In some embodiments, the gene is two or more genes. In some embodiments, the gene is three or more genes. In some embodiments, the mutation is two or more mutations. In some embodiments, the mutation is three or more mutations.

For the sake of brevity, the some of the methods above are directed to identifying a subject at elevated risk of developing IMRD or having undiagnosed IMRD. It is to be understood that other diseases are also contemplated, such as SLE or an SLE-related disease or a sub-type of IMRD such as ANA-positive or speckled ANA-positive IMRD.

### BRIEF DESCRIPTION OF DRAWINGS

FIGS. 1A-1E shows graphs depicting that regions on chromosome 11 and 32 are associated with ANA IMRD, and in particular with ANA speckled (ANA^{s}) IMRD, and that PTPN3, DDIT4L and BANK1 show differences in expression levels in peripheral blood mononuclear cells purified from healthy Nova Scotia duck tolling retriever (NSDTR) dogs depending on the SNP identities of the dog. (FIG. 1A) The association to the region on chromosome 11 was only slightly stronger in the ANA^{S} dogs with the MHC risk genotype 2 (N=25) (dots) compared with the complete sample of ANA dogs with this MHC genotype (N=63) (crosses), suggesting that this locus is important for both ANA^{S} and all ANA IMRD dogs. Number of controls used was 145. The black bar represents the highly associated 15 SNP haplotype and squares show SNPs correlated with expression. (FIG. 1B) The region on chromosome 32 showed a much stronger association in ANA^{S} dogs with the MHC risk genotype 2 (N=25) (dots) than in the complete sample of ANA dogs (N=63) (crosses), suggesting that this region is mostly affecting ANA^{S} IMRD. Number of controls used was 145. Squares indicate correlation with expression and large circles show no correlation with expression. (FIG. 1C) The log-transformed mRNA levels of PTPN3 in dogs with different haplotypes comprised of two synonymous SNPs in exon 3 and 18, and SNPs in intron 18 and the 3′-UTR. The protective haplotype is T/T-C/C-A/A-C/C while the only risk haplotype present among the healthy dogs was heterozygous C/T-A/C-A/G-T/T. The mRNA levels of DDIT4L (FIG. 1D) and BANK1 genes (FIG. 1E) stratified according to the haplotypes made by the top associated SNP 32:24,556,037 and 32:25,485,961, where A/A-G/G is protective and G/G-A/A is risk. Boxes represent interquartile range 25-75% with median, and 5-95 percentile range with maximum and minimum values. The gene expression was normalized to the levels of the housekeeping gene TBP and analyzed using a one-way ANOVA. The y-axis of FIG. 1C shows the “-log of relative mRNA levels”. The x-axis of FIG. 1C shows the following three genotypes from left to right: T/T-C/C-A/A-C/C; T/T-C/C/-A/A/-T/T; and C/T-A/C-A/G-T/T. The y-axis of FIG. 1D shows the “relative mRNA levels”. The x-axis of FIG. 1D shows the following three genotypes from left to right: A/A-G/G; A/G-G/G; and G/G-A/A. The y-axis of FIG. 1E shows the “relative mRNA levels”. The x-axis of FIG. lE shows the following three genotypes from left to right: A/A-G/G; A/G-G/G; and G/G-A/A.

FIG. 2 is a multi-locus chart of ANA^{s} dogs with the three risk loci at MHC class II, chromosome 11 and chromosome 32.

FIG. 3 is a multi-locus chart of controls with the three risk loci at MHC class II, chromosome 11 and chromosome 32.

FIG. 4 is a graph of the expression of genes from the chromosome 32 locus. The genes are from left to right: DAPP1, LAMTOR3, DNAJB14, H2AFZ, DDIT4L, EMCN, PPP3CA, and BANK1.

FIG. 5 is an R-square chart showing SNP associations of chromosome 11 for ANA^{s} dogs. The level of LD is indicated, with crosses representing a high LD. The top SNP is shown as a square. The r² values are represented using the following symbols: cross=1-0.95, triangle=0.53, circle=0.2-0.3, and dot=<0.2.

FIG. 6A is a D-prime plot of the region of chromosome 11. The SNPs on the top of the plot from left to right are: 11:67463150, 11:67465332, 11:67479814, 11:67481323, 11:67484477, 11:67485866, 11:67504858, 11:67511882, 11:67514454, 11:6756041, 11:67517102, 11:67518063, 11:67518596, 11:67518596, 11:67518781, 11:67519533, 11:67520723, 11:67523597, 11:67527627, 11:67531399, 11:67535953, 11:67536642, 11:67536944, 11:67537177, 11:67537363, 11:67537493, 11:67537924, 11:67538032, 11:67538806, 11:67539578, 11:67539780, 11:67539967, 11:67543652, 11:67553409, 11:67554201, 11:67557371, 11:67560132, 11:67565265, 11:67570186, 11:67576318, 11:67576585, 11:67577041, 11:67583114, 11:67583604, 11:67583604, and 11:67583635.

FIG. 6B is a D-prime plot of a 16 SNP haplotype on chromosome 11. The 16 SNP haplotype in high LD was shown to correlate to expression of PTPN3 by the four tagged SNPs at circled positions, which are 11:67516041, 11:67538032, 11:67538806, and 11:67583604.

FIG. 7 is a graph showing the correlation of PTPN3 expression with a three SNP haplotype on chromosome 11.

FIG. 8 is an R-squared chart showing SNP associations on chromosome 32 for ANA^{s} dogs. The level of LD is indicated, with crosses representing a high LD. The top SNP is shown as a square. The r² values are represented using the following symbols: cross=0.8-1.0, triangle=0.4-0.6 and 0.6-0.8, circle=0.2-0.4, and dot=<0.2.

### DETAILED DESCRIPTION OF INVENTION

SLE is a chronic systemic autoimmune disorder in which the immune system of a subject attacks the cells and tissue of the body, resulting in tissue damage and inflammation. SLE can affect any part of the body, but most typically affects the heart, joints, skin, lungs, blood vessels, liver, kidneys, and nervous system. SLE often consists of alternating periods of illness and remission. While SLE and SLE-related diseases were first described in human patients, they are also seen in other species including dogs with similar clinical manifestations [refs. 3, 6, 7]. The most common clinical signs shown in dogs are polyarthritis, fever, anemia, skin problems, and rarely renal failure [refs. 3,8]. In canine subjects, such as Nova Scotia duck tolling retriever (NSDTR) dogs, an SLE-like disease called immune-mediated rheumatic disease (IMRD) can develop. Methods for identifying subjects at risk for developing SLE or SLE-related diseases such as IMRD would have significant medical benefit.

Aspects of the invention relate to germ-line risk markers (such as single nucleotide polymorphisms (SNPs), risk haplotypes, and mutations in genes) and various methods of use and/or detection thereof. The invention is premised, in part, on the results of a genomic analysis conducted using NSDTR dogs having different sub-types of antinuclear antibody (ANA) positive IMRD. The study is described herein. Briefly, SNPs in regions on chromosomes 11 and 32 were identified as being associated with ANA-positive IMRD, and highly associated with speckled ANA-positive IMRD. SNPs on chromosomes 11 and 32 identified as associated with speckled ANA-positive IMRD were found to correlate with decreased expression of PTPN3 and increased expression of DDIT4L and BANK1, indicating that the expression levels of these genes may correlate with the presence of IMRD, such as speckled ANA-positive IMRD.

Accordingly, aspects of the invention provide methods that involve detecting one or more of the identified germ-line risk markers in a subject, e.g., a canine subject, in order to (a) identify a subject at elevated risk of developing SLE or an SLE-related disease such as IMRD, or (b) identify a subject having SLE or an SLE-related disease such as IMRD that is as yet undiagnosed. The methods can be used for prognostic purposes and for diagnostic purposes. Identifying canine subjects having an elevated risk of developing SLE or an SLE-related disease such as IMRD is useful in a number of applications. For example, canine subjects identified as at elevated risk may be excluded from a breeding program and/or conversely canine subjects that do not carry the germ-line risk markers may be included in a breeding program (e.g., selected as breeding dogs in order to minimize unfavorable combinations of genetic risk factors). As another example, canine subjects identified as at elevated risk may be monitored, including monitored more regularly, for the appearance of SLE or an SLE-related disease such as IMRD and/or may be treated prophylactically (e.g., prior to the development of the disease) or therapeutically. Canine subjects carrying one or more of the germ-line risk markers may also be used to further study the progression of SLE or an SLE-related disease such as IMRD and optionally to study the efficacy of various treatments.

In addition, in view of the clinical similarity between canine IMRD with human SLE, the germ-line risk markers, such as risk-associated regions and/or genes, identified in accordance with the invention may also be or may contain risk markers and/or mediators of human SLE. Accordingly, the invention provides diagnostic and prognostic methods for use in canine subjects, animals more generally, and human subjects, as well as animal models of human disease and treatment, as well as others.

### Elevated Risk of Developing SLE and SLE-Related Diseases

The germ-line risk markers of the invention can be used to identify subjects at elevated risk of developing SLE or an SLE-related disease such as IMRD. An elevated risk means a lifetime risk of developing SLE or an SLE-related disease such as IMRD that is higher than the risk of developing the same disease in (a) a population that is unselected for the presence or absence of the germ-line risk marker and/or up- or down-regulated expression of genes such as PTPN3, DDIT4L and BANK1 (i.e., the general population) or (b) a population that does not carry the germ-line risk marker or has an expression of genes such as PTPN3, DDIT4L and BANK1 that is similar to a control level.

### SLE, SLE-Related Diseases, and Diagnostic/Prognostic Methods

Aspects of the invention include various methods, such as prognostic and diagnostic methods, related to SLE and SLE-related diseases such as IMRD.

SLE tends to be clinically heterogenous, with manifestations ranging from relatively mild symptoms such as skin rash to severe impairment of functions of kidney, heart, lung, central nervous system and other organs. While SLE and SLE-related diseases were first described in human patients, they are also seen in other species including dogs with similar clinical manifestations [ref. 3, 6, 7]. The most common clinical signs shown in dogs are polyarthritis, fever, anemia, skin problems, and rarely renal failure [ref. 3, 8]. One such SLE-related disease is called immune-mediated rheumatic disease (IMRD).

A hallmark of SLE is the production of auto-antibodies directed to several self-molecules found in the nucleus, cytoplasm or on cell surface. Antinuclear antibodies (ANA) are found in more than 95% of SLE cases [ref. 5]. In both humans and dogs, a variety of ANAs detectable in serum have been correlated to SLE and certain SLE-related diseases such as IMRD [refs. 1, 7, 9]. The specific pattern of nucleoplasmic immunofluorescence such as a homogeneous staining (with a concomitant chromosomal reactivity) or speckled distribution of ANAs may be indicative of the presence of particular types of ANAs. Specific ANAs have been linked to sub-types of disease [refs. 1, 3, 8, and 10]. A recent study showed that among canine IMRD cases positive for ANA, 61% showed a speckled pattern (ANA^{S}) whereas 39% displayed homogeneous staining pattern (ANA^{H}) [ref. 3]. The staining phenotype was consistent during the course of the disease [ref. 3]. ANA-positive diseases, such as IMRD, have been shown to be prevalent in Nova Scotia duck tolling retriever (NSDTR) dogs and German shepherd dogs. Prior to the invention, distinction between speckled ANA-positive disease and other types of ANA-positive disease was difficult.

Accordingly, germ-line risk markers and expression levels of particular genes described herein can be used to (a) identify a subject at elevated risk of developing SLE or an SLE-related disease such as IMRD, or (b) identify a subject having SLE or an SLE-related disease such as IMRD that is as yet undiagnosed. In some aspects, the invention provides methods to (a) identify a subject at elevated risk of developing SLE or an SLE-related disease such as IMRD, or (b) identify a subject having SLE or an SLE-related disease such as IMRD that is as yet undiagnosed. In some aspects, the invention provides methods to (a) identify a subject at elevated risk of developing a sub-type of IMRD or (b) identify a subject having sub-type of IMRD that is as yet undiagnosed. In some embodiments, the sub-type of IMRD is ANA-positive IMRD. In some aspects, the invention provides methods to (a) identify a subject at elevated risk of developing a sub-type of ANA-positive IMRD or (b) identify a subject having sub-type of ANA-positive IMRD that is as yet undiagnosed. In some embodiments, the sub-type of ANA-positive IMRD is speckled ANA-positive IMRD. Speckled ANA-positive IMRD is a sub-type of IMRD characterized by a speckled pattern of staining, e.g., indirect immunofluorescence staining. A speckled pattern of ANA can be identified using methods known in the art and described herein [see, e.g., refs. 3 and 8, which are incorporated herein by reference in their entirety].

Available methods for diagnosis of SLE and SLE-related diseases include detection of ANAs, e.g., using indirect immunofluorescence (IIF). Subtypes of ANAs include anti-Smith and anti-double stranded DNA (dsDNA) antibodies, which have been shown to be associated with SLE. Other ANAs that may be used include anti-U1 RNP, anti-Ro, and anti-La antibodies. Other tests routinely performed to aid in diagnosis of SLE include measurement of complement system levels (low levels suggest consumption of C3 and C4 by the immune system), electrolytes and renal function (disturbed if the kidney is involved), liver enzymes, and complete blood count.

Thus, in some embodiments, the prognostic or diagnostic methods of the invention may further comprise performing a diagnostic assay known in the art for identification of SLE or an SLE-related diseases such as IMRD.

### Germ-Line Risk Markers

Aspects of the invention relate to germ-line risk markers and use and detection thereof in various methods. In general terms, a germ-line marker is a mutation in the genome of a subject that can be passed to the offspring of the subject. Germ-line markers may or may not be risk markers. Germ-line markers are generally found in the majority, if not all, of the cells in a subject. Germ-line markers are generally inherited from one or both parents of the subject (i.e., were present in the germ cells of one or both parents). Germ-line markers, as used herein, also include de novo germ-line mutations, which are spontaneous mutations that occur at the single-cell stage during embryonic development. This is distinct from a somatic marker, which is a mutation in the genome of a subject that occurs after the single-cell stage during development. Somatic mutations are considered to be spontaneous mutations. Somatic mutations generally originate in a single cell or subset of cells in the subject.

A germ-line risk marker, as used herein, is a germ-line marker that is associated with an elevated risk of developing SLE or an SLE-related disease such as IMRD. Examples of germ-line risk markers include a SNP, a risk haplotype, or a mutation in a gene. Further discussion of each type of germ-line risk marker is provided herein.

As used herein, a mutation is one or more changes in the nucleotide sequence of the genome of the subject. The terms mutation, alteration, variation, and polymorphism are used interchangeably herein. As used herein, mutations include, but are not limited to, point mutations, insertions, deletions, rearrangements, inversions and duplications. Mutations also include, but are not limited to, silent mutations, missense mutations, and nonsense mutations.

### Single Nucleotide Polymorphisms (SNPs)

In some embodiments, a germ-line risk marker is a single nucleotide polymorphism (SNP). A SNP is a mutation that occurs at a single nucleotide location on a chromosome. The nucleotide located at that position may differ between individuals in a population and/or paired chromosomes in an individual. In some embodiments, a germ-line risk marker is a SNP selected from Table 1. Table 1 provides the risk nucleotide identity for each SNP (see “risk allele” column). The risk nucleotide is the nucleotide identity that is associated with elevated risk of developing SLE or an SLE-related disease such as IMRD or having undiagnosed SLE or an SLE-related disease such as IMRD. The position (i.e., the chromosome coordinates) for each SNP in Table 1 are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819, which is incorporated herein by reference in its entirety). The first base pair in each chromosome is labeled 0 and the position of the SNP is then the number of base pairs from the first base pair (for example, the SNP on chromosome 11 at position 67536642 is located 67536642 base pairs from the first base pair of chromosome 11).

TABLE 1
List of SNPs associated with elevated risk of IMRD

| | | RISK |
|---|---|---|
| CHROMOSOME | POSITION | ALLELE |
| 11 | 67536642 | C |
| 11 | 67535953 | A |
| 11 | 67543652 | A |
| 11 | 67538032 | A |
| 11 | 67516041 | C |
| 11 | 67537177 | G |
| 11 | 67537363 | A |
| 11 | 67538806 | G |
| 11 | 67537493 | A |
| 11 | 67485866 | A |
| 11 | 67504858 | A |
| 11 | 67518596 | A |
| 11 | 67518781 | A |
| 11 | 67536944 | A |
| 11 | 67537924 | A |
| 11 | 67511882 | C |
| 11 | 67583604 | T |
| 32 | 24556037 | G |
| 32 | 24667283 | A |
| 32 | 25537276 | G |
| 32 | 25392401 | A |
| 32 | 24606503 | A |
| 32 | 24650093 | A |
| 32 | 25798353 | G |
| 32 | 25485961 | A |
| 32 | 25007496 | G |
| 32 | 25007632 | C |
| 32 | 25642357 | T |
| 32 | 25485644 | A |
| 32 | 24672221 | A |
| 32 | 25702963 | G |
| 32 | 24618331 | C |
| 32 | 25049586 | A |
| 32 | 25779083 | C |
| 32 | 25484844 | A |
| 32 | 25816401 | A |
| 32 | 25718852 | G |
| 32 | 25305524 | G |
| 32 | 25710678 | C |
| 32 | 25662984 | A |

In some embodiments, the SNP may be one or more of i) one or more chromosome 11 SNP or ii) one or more chromosome 32 SNPs, all of which are provided in Table 1.

In some embodiments, a SNP may be used in the methods described herein. In some embodiments, the method comprises:

a) analyzing genomic DNA from a canine subject for the presence of a SNP selected from i) one or more chromosome 11 SNP and ii) one or more chromosome 32 SNPs; and

b) identifying the canine subject having one or more of the SNPs as a subject (a) at elevated risk of developing SLE or an SLE-related disease such as IMRD or (b) having undiagnosed SLE or an SLE-related disease such as IMRD.

In some embodiments, the SNP is selected from a SNP at chromosome position chr11:67536642, chr11:67535953, chr11:67543652, chr11:67538032, chr11:67516041, chr11:67537177, chr11:67537363, chr11:67538806, chr11:67537493, chr11:67485866, chr11:67504858, chr11:67518596, chr11:67518781, chr11:67536944, chr11:67537924, chr11:67511882, or chr11:67583604. In some embodiments, the SNP is a SNP at chromosome position chr11:67583604.

In some embodiments, the SNP is selected from a SNP at chromosome position chr32:24556037, chr32:24667283, chr32:25537276, chr32:25392401, chr32:24606503, chr32:24650093, chr32:25798353, chr32:25485961, chr32:25007496, chr32:25007632, chr32:25642357, chr32:25485644, chr32:24672221, chr32:25702963, chr32:24618331, chr32:25049586, chr32:25779083, chr32:25484844, chr32:25816401, chr32:25718852, chr32:25305524, chr32:25710678, or chr32:25662984. In some embodiments, the SNP is selected from a SNP at chromosome position chr32:24556037, chr32:25485961, or chr32:25485644. In some embodiments, the SNP is a SNP at chromosome position chr32:24556037.

It is to be understood that any number of SNPs (e.g., 1, 2, 3, 4, 5, 6, 7, 8, 9, 10, 11, 12, 13, 14, 15, 16, 17, 18, 19, or 20 or more SNPs) on either chromosome 11, 32, or both may be detected and/or used to identify a subject.

### Risk Haplotypes

In some embodiments, a germ-line risk marker is a risk haplotype. A risk haplotype, as used herein, is a chromosomal region containing at least one mutation that correlates with the presence of or likelihood of developing SLE or an SLE-related disease such as IMRD in a subject. A risk haplotype is detected or identified and/or may be defined by one or more mutations. For example, a risk haplotype may be a chromosomal region with boundaries that are defined by two or more SNPs that are in linkage disequilibrium with each other and correlate with the presence or likelihood of developing SLE or an SLE-related disease such as IMRD in a subject. Such SNPs may themselves be disease-causative or may, alternatively or additionally, be indicators of other mutations present in the chromosomal region of the risk haplotype that correlate with or cause SLE or an SLE-related disease such as IMRD in a subject. Thus, other mutations within the risk haplotype may correlate with presence of or likelihood of developing SLE or an SLE-related disease such as IMRD in a subject and are contemplated for use in the methods herein as well.

Accordingly, in some embodiments, methods described herein comprise use and/or detection of a risk haplotype. In some embodiments, the risk haplotype is selected from a risk haplotype having chromosome coordinates chr11:67536642-67583604 or a risk haplotype having chromosome coordinates chr32:24556037-25816401.

Any chromosomal coordinates described herein are meant to be inclusive (i.e., include the boundaries of the chromosomal coordinates). In some embodiments, the risk haplotype may include additional chromosomal regions flanking the chromosomal regions described above, e.g., an additional 0.1, 0.5, 1, 2, 3, 4 or 5 Mb. In some embodiments, the risk haplotype may be a shortened chromosomal region relative to the chromosomal regions described above, e.g., 0.1, 0.5, or 1 Mb fewer than the chromosomal regions described above.

Any mutation of any size located within or spanning the chromosomal boundaries of a risk haplotype is contemplated herein for detection of the risk haplotype, e.g., a SNP, a deletion, an inversion, a translocation, or a duplication. In some embodiments, the risk haplotype is detected by analyzing the chromosomal region of the risk haplotype for the presence of a SNP. In some embodiments, a SNP in a risk haplotype is a SNP described in Table 1. It is to be understood that other SNPs not listed in Table 1 but located within the risk haplotype coordinates on chromosome 11 and/or 32 described above are also contemplated herein. In some embodiments the SNP is selected from a SNP at chromosome position chr11:67536642, chr11:67535953, chr11:67543652, chr11:67538032, chr11:67516041, chr11:67537177, chr11:67537363, chr11:67538806, chr11:67537493, chr11:67485866, chr11:67504858, chr11:67518596, chr11:67518781, chr11:67536944, chr11:67537924, chr11:67511882, chr11:67583604, chr32:24556037, chr32:25485961, or chr32:25485644.

In some embodiments, a risk haplotype can be used in the methods described herein. In some embodiments, the method comprises:

(a) analyzing genomic DNA from a canine subject for the presence of a risk haplotype selected from a risk haplotype having chromosome coordinates chr11:67536642-67583604 and a risk haplotype having chromosome coordinates chr32:24556037-25816401; and

(b) identifying a canine subject having the risk haplotype as a subject at elevated risk of developing SLE or an SLE-related disease such as IMRD or having an undiagnosed SLE or an SLE-related disease such as IMRD.

It is to be understood that any number of mutations (e.g., 1, 2, 3, 4, 5, 6, 7, 8, 9, 10, 11, 12, 13, 14, 15, 16, 17, 18, 19, or 20 or more mutations) can exist within each risk haplotype. It is also to be understood that not all mutations within the risk haplotype must be detected in order to determine that the risk haplotype is present or to make a diagnosis. For example, one mutation may be used to detect the presence of a risk haplotype. In another example, two or more mutations may be used to detect and/or confirm the presence of a risk haplotype. It is also to be understood that subject identification may involve any number of risk haplotypes (e.g., 1, 2, 3, 4, or 5 risk haplotypes).

In some embodiments, the presence of a risk haplotype is determined by detecting one or more SNPs within the chromosomal coordinates of the risk haplotype. In some embodiments, the presence of the risk haplotype is detected by analyzing the genomic DNA for the presence of one or more SNPs in Table 1 within the chromosomal coordinates of the risk haplotype.

It is to be understood that any number of SNPs (e.g., 1, 2, 3, 4, 5, 6, 7, 8, 9, 10, 11, 12, 13, 14, 15, 16, 17, 18, 19, or 20 or more SNPs) in any number of risk haplotypes (e.g., 1 or 2 or more risk haplotypes) may be used. In some embodiments, a subset or all SNPs in Table 1 located within a risk haplotype are used to detect the presence of the risk haplotype.

### Genes

In some embodiments, a germ-line risk marker is a mutation in a gene. As used herein, a gene may include both coding and non-coding nucleotide sequences. As such, a gene may include any regulatory sequences (e.g., any promoters, enhancers, or suppressors, either adjacent to or far from the coding sequence) and any coding sequences. As used herein, a coding sequence includes the first DNA nucleotide to the last DNA nucleotide that is transcribed into an mRNA that includes the untranslated regions (UTRs), exons, and introns. The coding sequence for each gene can be obtained using the Ensembl database by entering the Ensembl gene IDs provided in Table 2, or by other methods known in the art. In some embodiments, the gene is contained within, near, or spanning the boundaries of a risk haplotype as described herein. In some embodiments, a mutation, such as a SNP, is contained within or near the gene. In some embodiments, the mutation is contained within or near the coding sequence of the gene. In some embodiments, the mutation is within 5000 kb, 2500 kb, 1000 kb, 900 kb, 800 kb, 700 kb, 600 kb, 500 kb, 400 kb, 300 kb, 200 kb, 150 kb, 100 kb, 50 kb, 25 kb, 10 kb, or 5 kb of a gene or of the coding sequence of the gene, as described herein. In some embodiments, the mutation is present in a gene selected from one or more genes located within a risk haplotype having chromosome coordinates chr11:67536642-67583604 or one or more genes located within a risk haplotype having chromosome coordinates chr32:24556037-25816401. In some embodiments, the mutation is present within the coding sequence of a gene selected from one or more genes located within a risk haplotype having chromosome coordinates chr11:67536642-67583604 or one or more genes located within a risk haplotype having chromosome coordinates chr32:24556037-25816401.

The mapped genes located within or near risk haplotypes on chromosome 11 and 32 are described in Table 2. The Ensembl gene identifiers are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). However, certain Ensembl Gene IDs in Table 2 are from CanFam3 as indicated by a “*” in Table 2. The Ensembl gene ID provided for each gene can be used to determine the nucleotide sequence of the gene, as well as associated transcript and protein sequences, by inputting the Ensembl ID into the Ensembl database (Ensembl release 70).

TABLE 2
Genes present in or near chromosomal regions associated
with elevated risk of IMRD

| | | | Associated |
|---|---|---|---|
| Gene | Canine Ensembl | Human Ensembl | Risk |
| Symbol | Gene ID | Gene ID | Haplotype |
| PTPN3 | ENSCAFG00000002868 | ENSG00000070159 | chr11: |
| | | | 67536642- |
| | | | 67583604 |
| DAPP1 | ENSCAFG00000010571 | ENSG00000070190 | chr32: |
| | | | 24556037- |
| | | | 25816401 |
| LAMTOR3 | ENSCAFG00000029975* | ENSG00000109270 | chr32: |
| | | | 24556037- |
| | | | 25816401 |
| DNAJB14 | ENSCAFG00000010592 | ENSG00000164031 | chr32: |
| | | | 24556037- |
| | | | 25816401 |
| H2AFZ | ENSCAFG00000010615 | ENSG00000164032 | chr32: |
| | | | 24556037- |
| | | | 25816401 |
| DDIT4L | ENSCAFG00000010626 | ENSG00000145358 | chr32: |
| | | | 24556037- |
| | | | 25816401 |
| EMCN | ENSCAFG00000032716* | ENSG00000164035 | chr32: |
| | | | 24556037- |
| | | | 25816401 |
| PPP3CA | ENSCAFG00000010644 | ENSG00000138814 | chr32: |
| | | | 24556037- |
| | | | 25816401 |
| BANK1 | ENSCAFG00000010676 | ENSG00000153064 | chr32: |
| | | | 24556037- |
| | | | 25816401 |

In some embodiments, a mutation in a gene is used in the methods described herein. In some embodiments, the method comprises:

(a) analyzing genomic DNA from a canine subject for the presence of a mutation in a gene selected from:

one or more genes located within a risk haplotype having chromosome coordinates chr11:67536642-67583604,

one or more genes located within a risk haplotype having chromosome coordinates chr32:24556037-25816401; and

(b) identifying a canine subject having the mutation as a subject at elevated risk of developing SLE or an SLE-related disease such as IMRD or having undiagnosed SLE or an SLE-related disease such as IMRD.

In some embodiments, the gene is selected from PTPN3, DAPP1, LAMTOR3, DNAJB14, H2AFZ, DDIT4L, EMCN, PPP3CA and BANK1. In some embodiments, the gene is selected from PTPN3, DAPP1, LAMTOR3, DNAJB14, H2AFZ, DDIT4L, EMCN, and PPP3CA. In some embodiments, the gene is selected from PTPN3, DDIT4L and BANK1. In some embodiments, the gene is selected from PTPN3 and DDIT4L. In some embodiments, the gene is selected from PTPN3 and BANK1.

In some embodiments, the gene is PTPN3. In some embodiments, PTPN3 is the nucleic acid sequence associated with the Ensembl ID ENSCAFG00000002868 as of the filing date of the instant application. In some embodiments, PTPN3 comprises the nucleic acid sequence associated with the Ensembl ID ENSCAFG00000002868 as of the priority date of the instant application. In some embodiments, PTPN3 comprises the nucleic acid sequence of SEQ ID NO: 83.

In some embodiments, the gene is BANK1. In some embodiments, BANK1 is the nucleic acid sequence associated with the Ensembl ID ENSCAFG00000010676 as of the filing date of the instant application. In some embodiments, BANK1 comprises the nucleic acid sequence associated with the Ensembl ID ENSCAFG00000010676 as of the priority date of the instant application. In some embodiments, BANK1 comprises the nucleic acid sequence of SEQ ID NO: 84.

Any number of mutations (e.g., 1, 2, 3, 4, 5, 6, 7, 8, 9, 10, 11, 12, 13, 14, 15, 16, 17, 18, 19, or 20 or more mutations) in any number of genes (e.g., 1, 2, 3, 4, 5, 6, 7, 8 or more genes) are contemplated.

The genes described herein can also be used to identify a subject at elevated risk of or having undiagnosed SLE or an SLE-related disease, where the subject is any of a variety of animal subjects including but not limited to human subjects. In some embodiments, the method, comprises

(a) analyzing genomic DNA in a sample from a subject for presence of a mutation in a gene selected from:

one or more genes located within a risk haplotype having chromosome coordinates chr11:67536642-67583604 or an orthologue of such a gene,

one or more genes located within a risk haplotype having chromosome coordinates chr32:24556037-25816401 or an orthologue of such a gene; and

(b) identifying a subject having the mutation as a subject at elevated risk of developing SLE or an SLE-related disease or having an undiagnosed SLE or an SLE-related disease.

In some embodiments, the subject is a human subject. In some embodiments, the subject is a canine subject. An orthologue of a gene may be, e.g., a human gene as identified in Table 2. In some embodiments, an orthologue of a gene has a sequence that is at least 70%, 75%, 80%, 85%, 90%, 95%, or 99% or more homologous to a sequence of the gene.

### Gene Expression Levels

The invention contemplates that elevated risk of developing SLE or an SLE-related disease such as IMRD or having undiagnosed SLE or an SLE-related disease such as IMRD is associated with an altered expression pattern of a gene located at, within, or near a risk haplotype, such as a gene located in Table 2. The invention therefore contemplates methods that involve measuring the mRNA or protein levels or protein activity levels for these genes and comparing such levels to control levels, including for example predetermined thresholds.

In some embodiments, the method comprises:

(a) analyzing a sample from a canine subject for a level of PTPN3, BANK1 and/or DDIT4L; and

(b) identifying a canine subject having a decreased level of PTPN3 and/or an elevated level of BANK1 and/or DDIT4L compared to a control level as a subject at elevated risk of developing SLE or an SLE-related disease such as IMRD or having an undiagnosed SLE or an SLE-related disease such as IMRD.

In some embodiments, the method comprises:

(a) analyzing a sample from a canine subject for a level of PTPN3 and/or DDIT4L; and

(b) identifying a canine subject having a decreased level of PTPN3 and/or an elevated level of DDIT4L compared to a control level as a subject at elevated risk of developing SLE or an SLE-related disease such as IMRD or having an undiagnosed SLE or an SLE-related disease such as IMRD.

As used herein, “an elevated level” means that the level of expression or activity is above a control level, such as a pre-determined threshold or an expression level or activity level of the same gene in a control sample. Control levels are described in detail herein. An elevated level includes a level that is, for example, 1%, 5%, 10%, 20%, 30%, 40%, 50%, 60%, 70%, 80%, 90%, 100%, 150%, 200%, 300%, 400%, 500% or more above a control level. An elevated level also includes increasing a phenomenon from a zero state (e.g., no or undetectable level in a sample) to a non-zero state (e.g., a detectable level in a sample).

As used herein, “a decreased level” means that the level of expression or activity is below a control level, such as a pre-determined threshold or an expression level or activity level of the same gene in a control sample. Control levels are described in detail herein. A decreased level includes a level that is, for example, 1%, 5%, 10%, 20%, 30%, 40%, 50%, 60%, 70%, 80%, 90%, 100%, 150%, 200%, 300%, 400%, 500% or more below a control level. A decreased level also includes decreasing a phenomenon from a non-zero state (e.g., a detectable level in a sample) to a zero state (e.g., no or undetectable level in a sample).

### MHC Class II Alleles

Aspects of the invention relate to use of major histocompatibility complex (MHC) class II alleles. In canines, the MHC class II genes are called dog leukocyte antigen (DLA) class II genes and consist of three polymorphic genes known as DLA-DRB 1, -DQA 1 and -DQB1 and one monomorphic gene DLA-DRA. The alleles for each DLA have been previously described [refs. 12, 13, and 16, which are incorporated herein by reference in their entirety]. The alleles for DLA-DRB1 include DLA-DRB1*01502, DLA-DRB1*00601, DLA-DRB1*01501, DLA-DRB1*02301, and DLA-DRB1*00401. The alleles for DLA-DQA1 include DLA-DQA1*00601, DLA-DQA1*005011, DLA-DQA1*00301, and DLA-DQA1*00201. The alleles for DLA-DQB1 include DLA-DQB1*02301, DLA-DQB1*02001, DLA-DQB1*00301, DLA-DQB1*00501, and DLA-DQB1*01501. As described herein, speckled ANA-positive IMRD was found to be associated with the DLA haplotype DLA-DRB1*00601, DQA1*005011, and DQB1*02001, and highly associated with homozygosity of the DLA haplotype DLA-DRB1*00601, DQA1*005011, and DQB1*02001.

Accordingly, aspects of the invention relate to use of DLA haplotypes. The identity of DLA haplotype in a genomic DNA sample can be determined, e.g., using a nucleic acid based method such as PCR or sequencing. Nucleic acid based methods are described herein.

The following nucleotide sequences are the fragments of each DLA allele (beginning at base 14 of exon 2 of each DLA allele). These sequences can be used to distinguish the different alleles.

>DLA-DRB1*00401
(SEQ ID NO: 1)
CACATTTCGTGTACCAGTTTAAGCCCGAGTGCCATTTCACCAACGGGAC
GGAGCGGGTGCGGTTCGTGGAAAGACACATCCATAACCGGGAGGAGTTC
GTGCGCTTCGACAGCGACGTGGGGGAGTACCGGGCGGTCACGGAGCTCG
GGCGGCCCGACGCTGAGTCCTGGAACGGGCAGAAGGAGCTCTTGGAGCA
GGAGCGGGCAACGGTGGACACCTACTGCAGACACAACTACGGGGTGATT
GAGAGCTTCACGGTGCAGCGGCGAG
>DLA-DRB1*00601
(SEQ ID NO: 2)
CACATTTCTTGGAGGTGGCAAAGGCCGAGTGCCATTTCACCAACGGGAC
GGAGCGGGTGCGGTTCGTGGAAAGATACATCTATAACCGGGAGGAGTAC
GTGCGCTTCGACAGCGACGTGGGGGAGTACCGGGCGGTCACGGAGCTCG
GGCGGCCCGACGCTGAGTACTGGAACCCGCAGAAGGAGCTCTTGGAGCG
GGCGCGGGCCGCGGTGGACACCTACTGCAGACACAACTACGGGGTGGGC
GAGAGCTTCACGGTGCAGCGGCGAG
>DLA-DRB1*01501
(SEQ ID NO: 3)
CACATTTCTTGGAGATGGTAAAGTTCGAGTGCCATTTCACCAACGGGAC
GGAGCGGGTGCGGCTTCTGGTGAGAGACATCTATAACCGGGAGGAGCAC
GTGCGCTTCGACAGCGACGTGGGGGAGTACCGGGCGGTCACGGAGCTCG
GGCGGCCCGACGCTGAGTACTGGAACGGGCAGAAGGAGCTCTTGGAGCA
GAGGCGGGCCGAGGTGGACACGGTGTGCAGACACAACTACGGGGTGATT
GAGAGCTTCACGGTGCAGCGGCGAG
>DLA-DRB1*01502
(SEQ ID NO: 4)
CACATTTCTTGGAGATGGTAAAGTTCGAGTGCCATTTCACCAACGGGAC
GGAGCGGGTGCGGCTTCTGGTGAGAGACATCTATAACCGGGAGGAGCAC
GTGCGCTTCGACAGCGACGTGGGGGAGTACCGGGCGGTCACGGAGCTCG
GGCGGCCCGACGCTGAGTACTGGAACGGGCAGAAGGAGCTCTTGGAGCA
GAGGCGGGCCGAGGTGGACACGGTGTGCAGACACAACTACGGGGTGATT
GAGAGCTTCGCGGTGCAGCGGCGAG
>DLA-DRB1*02301
(SEQ ID NO: 5)
CACATTTCTTGGAGATGTTAAAGTTCGAGTGCCATTTCACCAACGGGAC
GGAGCGGGTGCGGTTCGTGGAAAGATACATCCATAACCGGGAGGAGTTC
GTGCGCTTCGACAGCGACGTGGGGGAGTACCGGGCGGTCACGGAGCTCG
GGCGGCCCGACGCTGAGTCCTGGAACCGGCAGAAGGAGCTCTTGGAGCA
GGAGCGGGCCGCGGTGGACACCTACTGCAGACACAACTACCGGGTGGGC
GAGAGCTTCACGGTGCAGCGGCGAG
>DLA-DQB1*00301
(SEQ ID NO: 6)
GATTTCGTGTACCAGTTTAAGGCCGAGTGCTATTTCACCAACGGGACGG
AGCGGGTGCGGCTTCTGACTAAATACATCTATAACCGGGAGGAGTACGT
GCGCTTCGACAGCGACGTGGGGGAGTACCGGGCGGTCACGGAGCTCGGG
CGGCCCTCGGCTGAGTACTGGAACCCGCAGAAGGACGAGATGGACCGGG
TACGGGCCGAGCTGGACACGGTGTGCAGACACAACTACGGGTTGGAAGA
GCTCACCACGTTGCAGCGGCGA
>DLA-DQB1*00501
(SEQ ID NO: 7)
GATTTCGTGTTCCAGTATAAGGCCGAGTGCTATTTCACCAACGGGACGG
AGCGGGTGCGGCTTCTGACTAAATACATCTATAACCGGGAGGAGTACGT
GCGCTTCGACAGCGACGTGGGGGAGTACCGGGCGGTCACGGAGCTCGGG
CGGCCCTGGGCTGAGTACTGGAACCCGCAGAAGGACGAGATGGACCGGG
TACGGGCCGAGCTGGACACGGTGTGCAGACACAACTACGGGTTGGAAGA
GCTCACCACGTTGCAGCGGCGA
>DLA-DQB1*01501
(SEQ ID NO: 8)
GATTTCGTGTACCAGTGTAAGGCCGAGTGCTATTTCACCAACGGGACGG
AGCGGGTGCGGTTTCTGGCTAAATACATCTATAACCGGGAGGAGTTCGT
GCGCTTCGACAGCGACGTGGGGGAGTTCCGGGCGGTCACGGAGCTCGGG
CGGCCCTCGGCTGAGTACTGGAACGGGCAGAAGGAGATCTTGGAGCAGG
AGCGGGCAACGGTGGACACGGTGTGCAGACACAACTACGGGGTGGAAGA
GCTCTACACGTTGCAGCGGCGA
>DLA-DQB1*02001
(SEQ ID NO: 9)
GATTTCGTGTACCAGTTTAAGGCCGAGTGCTATTTCACCAACGGGACGG
AGCGGGTGCGGCTTCTGACGAGAAGCATCTATAACCGGGAGGAGTTCGT
GCGCTTCGACAGCGACGTGGGGGAGTTCCGGGCGGTCACGGAGCTCGGG
CGGCCCGTCGCTGAGTACTGGAACGGGCAGAAGGAGATCTTGGAGCGGA
AGCGGGCCGCGGTGGACACGGTGTGCAGACACAACTACGGGAGGGAAGA
GCTCACCACGTTGCAGCGGCGA
>DLA-DQB1*02301
(SEQ ID NO: 10)
GATTTCGTGTACCAGTTTAAGGGCGAGTGCTATTTCACCAACGGGACGG
AGCGGGTGCGGCTTCTGACTAAATACATCTATAACCGGGAGGAGTACGT
GCGCTTCGACAGCGACGTGGGGGAGTACCGGGCGGTCACGGAGCTCGGG
CGGCCCTCGGCTGAGTACTGGAACCCGCAGAAGGACGAGATGGACCGGG
TACGGGCCGAGCTGGACACGGTGTGCAGACACAACTACGGGTTGGAAGA
GCTCACCACGTTGCAGCGGCGA
>DLA-DQA1*00201
(SEQ ID NO: 11)
GACCATGTTGCCTACTACGGCATAAATGTCTACCAGTCTTACGGTCCCT
CTGGCCAGTACACCCATGAATTTGATGGCGATGAGGAGTTCTACGTGGA
CCTGGAGAAGAAGGAAACTGTCTGGCGGCTGCCTGTGTTTAGCACATTT
ACAAGTTTTGACCCACAGGGTGCACTGAGAAACTTGGCTATAACAAAAC
AAAACTTGAACATCATGACTAAAAGGTCCAACAAAACTGCTGCTACCAA
T
>DLA-DQA1*00301
(SEQ ID NO: 12)
GACCATGTTGCCTACTACGGCATAAATGTCTACCAGTCTTACGGTCCCT
CTGGCCAGTACACCCATGAATTTGATGGCGATGAGGAGTTCTACGTGGA
CCTGGAGAAGAAGGAAACTGTCTGGCGGCTGCCTGTGTTTAGCACATTT
ACAAGTTTTGACCCACAGGGTGCACTGAGAAACTTGGCCAGAGCAAAAC
AAAACTTGAACATCCTGACTAAAAGTTCCAACCAAACTGCTGCTACCAA
T
>DLA-DQA1*005011
(SEQ ID NO: 13)
GACCATGTTGCCTACTACGGCATAAATGTCTACCAGTCTTACGGTCCCT
CTGGCCAGTTCACCCATGAATTTGATGGCGATGAGGAGTTCTACGTGGA
CCTGGAGAAGAAGGAAACTGTCTGGCGGCTGCCTGTGTTTAGCACATTT
ACAAGTTTTGACCCACAGGGTGCACTGAGAAACTTGGCTATAACAAAAC
AAAACTTGAACATCATGACTAAAAGGTCCAACAAAACTGCTGCTACCAA
T
>DLA-DQA1*00601
(SEQ ID NO: 14)
GACCATGTTGCCTACTACGGCATAAATGTCTACCAGTCTTACGGTCCCT
CTGGCCAGTACACCCATGAATTTGATGGCGATGAGGAGTTCTACGTGGA
CCTGGAGAAGAAGGAAACTGTCTGGCGGCTGCCTGTGTTTAGCACATTT
AGAAGTTTTGACCCACAGGGTGCACTGAGAAACTTGGCTATAATAAAAC
AAAACTTGAACATCCTGACTAAAAGGTCCAACCAAACTGCTGCTACCAA
T

In some embodiments, methods of the invention comprise analyzing genomic DNA for the presence of a DLA haplotype. In some embodiments, the DLA haplotype is DLA-DRB1*00601, DQA1*005011, and DQB1*02001. In some embodiments, a subject heterozygous or homozygous for the DLA haplotype is identified as a subject at elevated risk of developing SLE or an SLE-related disease such as IMRD or having undiagnosed SLE or an SLE-related disease such as IMRD. In some embodiments, a subject homozygous for the DLA haplotype is identified as a subject at elevated risk of developing SLE or an SLE-related disease such as IMRD or having undiagnosed SLE or an SLE-related disease such as IMRD. In some embodiments, the SLE or an SLE-related disease is IMRD. In some embodiments, the IMRD is ANA-positive IMRD. In some embodiments, the ANA-positive IMRD is speckled ANA-positive IMRD.

In some embodiments, methods of the invention can combine analysis of the DLA haplotype with analysis of a germ-line marker of the invention. In some embodiments, the method, comprising:

a) analyzing genomic DNA from a canine subject for the presence of a single nucleotide polymorphism (SNP) selected from i) one or more chromosome 11 SNPs and ii) one or more chromosome 32 SNPs;

b) analyzing the genomic DNA for the presence of a DLA haplotype DLA-BRB1*00601, DQA1*005011, and DQB1*02001; and

c) identifying a canine subject having the SNP and homozygous for the DLA haplotype as a subject at elevated risk of developing SLE or an SLE-related disease such as IMRD or having an undiagnosed SLE or an SLE-related disease such as IMRD.

In some embodiments, the method comprises:

a) analyzing genomic DNA from a canine subject for the presence of a risk haplotype selected from a risk haplotype having chromosome coordinates chr11:67536642-67583604 and a risk haplotype having chromosome coordinates chr32:24556037-25816401;

b) analyzing the genomic DNA for the presence of a DLA haplotype DLA-BRB1*00601, DQA1*005011, and DQB1*02001; and

c) identifying a canine subject having the risk haplotype and homozygous for the DLA haplotype as a subject at elevated risk of developing SLE or an SLE-related disease such as IMRD or having an undiagnosed SLE or an SLE-related disease such as IMRD.

In some embodiments, the method comprises:

(a) analyzing genomic DNA from a canine subject for the presence of a mutation in a gene selected from:

one or more genes located within a risk haplotype having chromosome coordinates chr11:67536642-67583604,

one or more genes located within a risk haplotype having chromosome coordinates chr32:24556037-25816401;

b) analyzing the genomic DNA for the presence of a DLA haplotype DLA-BRB1*00601, DQA1*005011, and DQB1*02001; and

(c) identifying a canine subject having the mutation and homozygous for the DLA haplotype as a subject at elevated risk of developing SLE or an SLE-related disease such as IMRD or having an undiagnosed SLE or an SLE-related disease such as IMRD.

### Genome Analysis Methods

Some methods provided herein comprise analyzing genomic DNA. In some embodiments, analyzing genomic DNA comprises carrying out a nucleic acid-based assay, such as a sequencing-based assay or a hybridization based assay. In some embodiments, the genomic DNA is analyzed using a single nucleotide polymorphism (SNP) array. In some embodiments, the genomic DNA is analyzed using a bead array. Methods of genetic analysis are known in the art. Examples of genetic analysis methods and commercially available tools are described below.

Affymetrix: The Affymetrix SNP 6.0 array contains over 1.8 million SNP and copy number probes on a single array. The method utilizes at a simple restriction enzyme digestion of 250 ng of genomic DNA, followed by linker-ligation of a common adaptor sequence to every fragment, a tactic that allows multiple loci to be amplified using a single primer complementary to this adaptor. Standard PCR then amplifies a predictable size range of fragments, which converts the genomic DNA into a sample of reduced complexity as well as increases the concentration of the fragments that reside within this predicted size range. The target is fragmented, labeled with biotin, hybridized to microarrays, stained with streptavidin-phycoerythrin and scanned. To support this method, Affymetrix Fluidics Stations and integrated GS-3000 Scanners can be used.

Illumina Infinium: Examples of commercially available Infinium array options include the 660W-Quad (>660,000 probes), the 1MDuo (over 1 million probes), and the custom iSelect (up to 200,000 SNPs selected by user). Samples begin the process with a whole genome amplification step, then 200 ng is transferred to a plate to be denatured and neutralized, and finally plates are incubated overnight to amplify. After amplification the samples are enzymatically fragmented using end-point fragmentation. Precipitation and resuspension clean up the DNA before hybridization onto the chips. The fragmented, resuspended DNA samples are then dispensed onto the appropriate BeadChips and placed in the hybridization oven to incubate overnight. After hybridization the chips are washed and labeled nucleotides are added to extend the primers by one base. The chips are immediately stained and coated for protection before scanning. Scanning is done with one of the two Illumina iScan™ Readers, which use a laser to excite the fluorophore of the single-base extension product on the beads. The scanner records high-resolution images of the light emitted from the fluorophores. All plates and chips are barcoded and tracked with an internally derived laboratory information management system. The data from these images are analyzed to determine SNP genotypes using Illumina's BeadStudio. To support this process, Biomek F/X, three Tecan Freedom Evos, and two Tecan Genesis Workstation 150s can be used to automate all liquid handling steps throughout the sample and chip prep process.

Illumina BeadArray: The Illumina Bead Lab system is a multiplexed array-based format. Illumina's BeadArray Technology is based on 3-micron silica beads that self-assemble in microwells on either of two substrates: fiber optic bundles or planar silica slides. When randomly assembled on one of these two substrates, the beads have a uniform spacing of ˜5.7 microns. Each bead is covered with hundreds of thousands of copies of a specific oligonucleotide that act as the capture sequences in one of Illumina's assays. BeadArray technology is utilized in Illumina's iScan System.

Sequenom: During pre-PCR, either of two Packard Multiprobes is used to pool oligonucleotides, and a Tomtec Quadra 384 is used to transfer DNA. A Cartesian nanodispenser is used for small-volume transfer in pre-PCR, and another in post-PCR. Beckman Multimeks, equipped with either a 96-tip head or a 384-tip head, are used for more substantial liquid handling of mixes. Two Sequenom pin-tool are used to dispense nanoliter volumes of analytes onto target chips for detection by mass spectrometry. Sequenom Compact mass spectrometers can be used for genotype detection.

In some embodiments, methods provided herein comprise analyzing genomic DNA using a nucleic acid sequencing assay. Methods of genome sequencing are known in the art. Examples of genome sequencing methods and commercially available tools are described below.

Illumina Sequencing: 89 GAIIx Sequencers are used for sequencing of samples. Library construction is supported with 6 Agilent Bravo plate-based automation, Stratagene MX3005p qPCR machines, Matrix 2-D barcode scanners on all automation decks and 2 Multimek Automated Pipettors for library normalization.

454 Sequencing: Roche® 454 FLX-Titanium instruments are used for sequencing of samples. Library construction capacity is supported by Agilent Bravo automation deck, Biomek FX and Janus PCR normalization.

SOLiD Sequencing: SOLiD v3.0 instruments are used for sequencing of samples. Sequencing set-up is supported by a Stratagene MX3005p qPCR machine and a Beckman SC Quanter for bead counting.

ABI Prism® 3730 XL Sequencing: ABI Prism® 3730 XL machines are used for sequencing samples. Automated Sequencing reaction set-up is supported by 2 Multimek Automated Pipettors and 2 Deerac Fluidics—Equator systems. PCR is performed on 60 Thermo-Hybaid 384-well systems.

Ion Torrent: Ion PGM™ or Ion Proton™ machines are used for sequencing samples. Ion library kits (Invitrogen) can be used to prepare samples for sequencing.

Other Technologies: Examples of other commercially available platforms include Helicos Heliscope Single-Molecule Sequencer, Polonator G.007, and Raindance RDT 1000 Rainstorm.

mRNA Assays

The art is familiar with various methods for analyzing mRNA levels. Examples of mRNA-based assays include but are not limited to oligonucleotide microarray assays, quantitative RT-PCR, Northern analysis, and multiplex bead-based assays.

Expression profiles of cells in a biological sample (e.g., blood) can be carried out using an oligonucleotide microarray analysis. As an example, this analysis may be carried out using a commercially available oligonucleotide microarray or a custom designed oligonucleotide microarray comprising oligonucleotides for all or a subset of the transcripts described herein. The microarray may comprise any number of the transcripts, as the invention contemplates that elevated risk may be determined based on the analysis of single differentially expressed transcripts or a combination of differentially expressed transcripts. The transcripts may be those that are up-regulated in samples carrying a germ-line risk marker (compared to a control sample that does not carry the germ-line risk marker), or those that are down-regulated in samples carrying a germ-line risk marker (compared to a control that does not carry the germ-line risk marker), or a combination of these. The number of transcripts measured using the microarray therefore may be 1, 2, 3, 4, 5, 6, 7, 8, or more transcripts encoded by a gene in Table 2. It is to be understood that such arrays may however also comprise positive and/or negative control transcripts such as housekeeping genes that can be used to determine if the array has been degraded and/or if the sample has been degraded or contaminated. The art is familiar with the construction of oligonucleotide arrays.

Commercially available gene expression systems include Affymetrix GeneChip microarrays as well as all of Illumina standard expression arrays, including two GeneChip 450 Fluidics Stations and a GeneChip 3000 Scanner, Affymetrix High-Throughput Array (HTA) System composed of a GeneStation liquid handling robot and a GeneChip HT Scanner providing automated sample preparation, hybridization, and scanning for 96-well Affymetrix PEGarrays. These systems can be used in the cases of small or potentially degraded RNA samples. The invention also contemplates analyzing expression levels from fixed samples (as compared to freshly isolated samples). The fixed samples include formalin-fixed and/or paraffin-embedded samples. Such samples may be analyzed using the whole genome Illumina DASL assay. High-throughput gene expression profile analysis can also be achieved using bead-based solutions, such as Luminex systems.

Other mRNA detection and quantitation methods include multiplex detection assays known in the art, e.g., xMAP® bead capture and detection (Luminex Corp., Austin, Tex.).

Another exemplary method is a quantitative RT-PCR assay which may be carried out as follows: mRNA is extracted from cells in a biological sample (e.g., blood) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). 5 μl of the RT reaction is used for quantitative PCR using SYBR Green PCR Master Mix and gene-specific primers, in triplicate, using an ABI 7300 Real Time PCR System.

mRNA detection binding partners include oligonucleotide or modified oligonucleotide (e.g. locked nucleic acid) probes that hybridize to a target mRNA. Probes may be designed using the sequences or sequence identifiers listed in Table 2. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

### Protein Assays

The art is familiar with various methods for measuring protein levels. Protein levels may be measured using protein-based assays such as but not limited to immunoassays, Western blots, Western immunoblotting, multiplex bead-based assays, and assays involving aptamers (such as SOMAmer™ technology) and related affinity agents.

A brief description of an exemplary immunoassay is provided here. A biological sample is applied to a substrate having bound to its surface protein-specific binding partners (i.e., immobilized protein-specific binding partners). The protein-specific binding partner (which may be referred to as a “capture ligand” because it functions to capture and immobilize the protein on the substrate) may be an antibody or an antigen-binding antibody fragment such as Fab, F(ab)2, Fv, single chain antibody, Fab and sFab fragment, F(ab′)₂, Fd fragments, scFv, and dAb fragments, although it is not so limited. Other binding partners are described herein. Protein present in the biological sample bind to the capture ligands, and the substrate is washed to remove unbound material. The substrate is then exposed to soluble protein-specific binding partners (which may be identical to the binding partners used to immobilize the protein). The soluble protein-specific binding partners are allowed to bind to their respective proteins immobilized on the substrate, and then unbound material is washed away. The substrate is then exposed to a detectable binding partner of the soluble protein-specific binding partner. In one embodiment, the soluble protein-specific binding partner is an antibody having some or all of its Fc domain. Its detectable binding partner may be an anti-Fc domain antibody. As will be appreciated by those in the art, if more than one protein is being detected, the assay may be configured so that the soluble protein-specific binding partners are all antibodies of the same isotype. In this way, a single detectable binding partner, such as an antibody specific for the common isotype, may be used to bind to all of the soluble protein-specific binding partners bound to the substrate.

It is to be understood that the substrate may comprise capture ligands for one or more proteins, including two or more, three or more, four or more, five or more, etc. up to and including all of the proteins encoded by the genes in Table 2 provided by the invention.

Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Protein detection binding partners include protein-specific binding partners. Protein-specific binding partners can be generated using the sequences or sequence identifiers listed in Table 2. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include non-antibody proteins or peptides that bind to or interact with a target protein, e.g., through non-covalent bonding. For example, if the protein is a ligand, a binding partner may be a receptor for that ligand. In another example, if the protein is a receptor, a binding partner may be a ligand for that receptor. In yet another example, a binding partner may be a protein or peptide known to interact with a protein. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target. Methods for producing aptamers to a target are known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, Colo.) modified nucleic acid-based protein binding reagents.

Binding partners also include any molecule capable of demonstrating selective binding to any one of the target proteins disclosed herein, e.g., peptoids (see, e.g., Reyna J Simon et al., “Peptoids: a modular approach to drug discovery” Proceedings of the National Academy of Sciences USA, (1992), 89(20), 9367-9371; U.S. Pat. No. 5,811,387; and M. Muralidhar Reddy et al., Identification of candidate IgG biomarkers for Alzheimer's disease via combinatorial library screening. Cell 144, 132-142, Jan. 7, 2011).

### Detectable Labels

Detectable binding partners may be directly or indirectly detectable. A directly detectable binding partner may be labeled with a detectable label such as a fluorophore. An indirectly detectable binding partner may be labeled with a moiety that acts upon (e.g., an enzyme or a catalytic domain) or a moiety that is acted upon (e.g., a substrate) by another moiety in order to generate a detectable signal. Exemplary detectable labels include, e.g., enzymes, radioisotopes, haptens, biotin, and fluorescent, luminescent and chromogenic substances. These various methods and moieties for detectable labeling are known in the art.

### Devices and Kits

Any of the methods provided herein can be performed on a device, e.g., an array. Suitable arrays are described herein and known in the art. Accordingly, a device, e.g., an array, for detecting any of the germ-line risk markers (e.g., 1, 2, 3, 4, 5, 6, 7, 8, 9, 10, or more germ-line risk markers, or at least 10, at least 20, at least 30, at least 40, at least 50, or more germ-line risk markers, or up to 3, up to 5, up to 10, up to 15, up to 20, up to 25, up to 30, up to 35, up to 40, up to 45, up to 50, up to 75 or up to 100 germ-line risk markers) described herein is also contemplated.

Reagents for use in any of the methods provided herein can be in the form of a kit. Accordingly, a kit for detecting any of the germ-line risk markers (e.g., 1, 2, 3, 4, 5, 6, 7, 8, 9, 10, or more germ-line risk markers, or at least 10, at least 20, at least 30, at least 40, at least 50, or more germ-line risk markers, or up to 3, up to 5, up to 10, up to 15, up to 20, up to 25, up to 30, up to 35, up to 40, up to 45, up to 50, up to 75 or up to 100 germ-line risk markers) described herein is also contemplated. In some embodiments, the kit comprises reagents for detecting any of the germ-line risk markers described herein, e.g., reagents for use in a method described herein. Suitable reagents are described herein and art known in the art.

### Controls

Some of the methods provided herein involve measuring a level of expression of a gene or determining the identity of a germ-line risk marker in a biological sample and then comparing that level or identity to a control in order to identify a subject having an elevated risk of developing SLE or an SLE-related disease, such as IMRD, or having undiagnosed SLE or an SLE-related disease, such as IMRD. The control may be a control level or identity that is a level or identity of the same gene or germ-line marker in a control tissue, control subject, or a population of control subjects.

The control may be (or may be derived from) a normal subject (or normal subjects). A normal subject, as used herein, refers to a subject that is healthy, such a subject experiencing none of the symptoms associate with SLE or an SLE-related disease. The control population may be a population of normal subjects.

In other instances, the control may be (or may be derived from) a subject who is negative for a germ-line risk marker described herein.

It is to be understood that the methods provided herein do not require that a control level or identity be measured every time a subject is tested. Rather, it is contemplated that control levels of expression of genes or control identities or germ-line risk markers are obtained and recorded and that any test level is compared to such a pre-determined level or identity (or threshold).

In some embodiments, a control is a nucleotide other than the risk nucleotide as described in Table 1.

### Samples

The methods provided herein detect and optionally measure (and thus analyze) particular germ-line risk markers or levels of expression genes in biological samples. Biological samples, as used herein, refer to samples taken or obtained from a subject. These biological samples may be tissue samples or they may be fluid samples (e.g., bodily fluid). Examples of biological fluid samples are whole blood, plasma, serum, urine, sputum, phlegm, saliva, tears, and other bodily fluids. In some embodiments, the biological sample is a whole blood or saliva sample. In some embodiments, the biological sample is skin.

In some embodiments, the biological sample may comprise a polynucleotide (e.g., genomic DNA or mRNA) derived from a tissue sample or fluid sample of the subject. In some embodiments, the biological sample may comprise a polypeptide (e.g., a protein) derived from a tissue sample or fluid sample of the subject. In some embodiments, the biological sample may be manipulated to extract a polynucleotide or polypeptide. In some embodiments, the biological sample may be manipulated to amplify a polynucleotide sample. Methods for extraction and amplification are well known in the art.

### Subjects

Methods of the invention are intended for canine subjects. In some embodiments, canine subjects include, for example, those with a higher incidence of SLE or an SLE-related disease such as IMRD as determined by breed. For example, the canine subject may be a Nova Scotia duck tolling retriever dog or a descendant of a Nova Scotia duck tolling retriever dog. However, it should be appreciated that other breeds may also be included as well. As used herein, a “descendant” includes any blood relative in the line of descent, e.g., first generation, second generation, third generation, fourth generation, etc., of a canine subject. Such a descendant may be a pure-bred canine subject, e.g., a descendant of two Nova Scotia duck tolling retriever dogs or a mixed-breed canine subject, e.g., a descendant of both a Nova Scotia duck tolling retriever dog and a non-Nova Scotia duck tolling retriever dog. Breed can be determined, e.g., using commercially available genetic tests (see, e.g., Wisdom Panel).

In some embodiments, a subject is homozygous for the DLA haplotype DLA-DRB1*00601, DQA1*005011, and DQB1*02001.

Methods of the invention may be used in a variety of other subjects including but not limited to human subjects.

### Computational Analysis

Methods of computation analysis of genomic and expression data are known in the art. Examples of available computational programs are: Genome Analysis Toolkit (GATK, Broad Institute, Cambridge, Mass.), Expressionist Refiner module (Genedata AG, Basel, Switzerland), GeneChip—Robust Multichip Averaging (CG-RMA) algorithm, PLINK (Purcell et al, 2007), GCTA (Yang et al, 2011), the EIGENSTRAT method (Price et al 2006), EMMAX (Kang et al, 2010). In some embodiments, methods described herein include a step comprising computational analysis.

### Breeding Programs

Other aspects of the invention relate to use of the diagnostic methods in connection with a breeding program. A breeding program is a planned, intentional breeding of a group of animals to reduce detrimental or undesirable traits and/or increase beneficial or desirable traits in offspring of the animals. Thus, a subject identified using the methods described herein as not having a germ-line risk marker of the invention may be included in a breeding program (e.g., selected as a breeding dog) to reduce the risk of developing SLE or an SLE-related disease such as IMRD in the offspring of said subject. Alternatively, a subject identified using the methods described herein as having a germ-line risk marker of the invention may be excluded from a breeding program. In some embodiments, methods of the invention comprise exclusion of a subject identified as being at elevated risk of developing SLE or an SLE-related disease such as IMRD or having undiagnosed SLE or an SLE-related disease such as IMRD in a breeding program or inclusion of a subject identified as not being at elevated risk of developing SLE or an SLE-related disease such as IMRD or having undiagnosed SLE or an SLE-related disease such as IMRD in a breeding program.

### Treatment

Other aspects of the invention relate to diagnostic or prognostic methods that comprise a treatment step (also referred to as “theranostic” methods due to the inclusion of the treatment step). Any treatment for SLE or an SLE-related disease such as IMRD is contemplated.

In some embodiments, treatment comprises administration of an effective amount of a corticosteroid, a non-steroidal anti-inflammatory drug, an immunomodulatory drug, or an anti-malarial drug.

In some embodiments, treatment comprises administration of an effective amount of a corticosteroid, such as prednisone or prednisolone. In some embodiments, treatment comprises administration of an effective amount of an immunomodulatory drug, such as azathioprine.

In some embodiment, treatment is palliative treatment. In some embodiments, palliative treatment comprises administering an effective amount of an analgesic.

It is to be understood that any treatment described herein may be used alone or may be used in combination with any other treatment described herein.

In some embodiments, a subject identified as being at elevated risk of developing SLE or an SLE-related disease such as IMRD or having undiagnosed SLE or an SLE-related disease such as IMRD is treated. In some embodiments, the method comprises selecting a subject for treatment on the basis of the presence of one or more germ-line risk markers or a level of expression of a gene as described herein. In some embodiments, the method comprises treating a subject with SLE or an SLE-related disease such as IMRD characterized by the presence of one or more germ-line risk markers or a level of expression of a gene as defined herein.

As used herein, “treat” or “treatment” includes, but is not limited to, preventing or reducing the development of SLE or an SLE-related disease such as IMRD and/or reducing the symptoms of SLE or an SLE-related disease such as IMRD.

An effective amount is a dosage of a therapy sufficient to provide a medically desirable result, such as treatment of SLE or an SLE-related disease such as IMRD. The effective amount will vary with the age and physical condition of the subject being treated, the severity of the condition, the duration of the treatment, the nature of any concurrent therapy, the specific route of administration and the like factors within the knowledge and expertise of the health practitioner.

Administration of a treatment may be accomplished by any method known in the art (see, e.g., Harrison's Principle of Internal Medicine, McGraw Hill Inc.). Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin). Dosage will depend on the subject and the route of administration. Dosage can be determined by the skilled artisan.

### EXAMPLES

### Example 1

As described in this example, the genetic risk factors related to the ANA^{S} subtype were identified and were correlated to gene expression changes. First, an indirect immunofluorescent ANA test was performed on serum from 64 cases and 78 control Nova Scotia duck tolling retrievers (NSDTRs). Of these, 26 cases were classified as having a homogenous staining pattern (ANA^{H}) and 32 cases as having a speckled pattern (ANA^{S}) (six cases could not be classified due to lack of serum), while all healthy controls were ANA-negative. For each of the DLA-DRB1, -DQA1 and -DQB1 genes, the polymorphic exon 2 was sequenced in all dogs (Table 3).

TABLE 3
Diagnostic information and DLA-DRB1, -DQA1 and -DQB1 alleles, haplotypes and
genotypes for all dogs included in the study.

| ID | Status | ANA | DRB1 | DQA1 | DQB1 | DRB1 | DQA1 | DQB1 | Haplotype | Genotype |
|---|---|---|---|---|---|---|---|---|---|---|
| 1 | Case | ANA^{H} | 01502 | 00601 | 02301 | 01502 | 00601 | 02301 | 1.1 | 1 |
| 2 | Case | ANA^{H} | 01502 | 00601 | 02301 | 01502 | 00601 | 02301 | 1.1 | 1 |
| 3 | Case | ANA^{H} | 01502 | 00601 | 02301 | 01502 | 00601 | 02301 | 1.1 | 1 |
| 4 | Case | ANA^{H} | 01502 | 00601 | 02301 | 01502 | 00601 | 02301 | 1.1 | 1 |
| 5 | Case | ANA^{H} | 01502 | 00601 | 02301 | 01502 | 00601 | 02301 | 1.1 | 1 |
| 6 | Case | ANA^{H} | 01502 | 00601 | 02301 | 01502 | 00601 | 02301 | 1.1 | 1 |
| 7 | Case | ANA^{H} | 01502 | 00601 | 02301 | 01502 | 00601 | 02301 | 1.1 | 1 |
| 8 | Case | ANA^{H} | 01502 | 00601 | 02301 | 01502 | 00601 | 02301 | 1.1 | 1 |
| 9 | Case | ANA^{H} | 01502 | 00601 | 02301 | 01502 | 00601 | 02301 | 1.1 | 1 |
| 10 | Case | ANA^{H} | 00601 | 005011 | 02001 | 01502 | 00601 | 02301 | 1.2 | 5 |
| 11 | Case | ANA^{H} | 01501 | 00601 | 00301 | 01502 | 00601 | 02301 | 1.3 | 5 |
| 12 | Case | ANA^{H} | 01501 | 00601 | 00301 | 01502 | 00601 | 02301 | 1.3 | 5 |
| 13 | Case | ANA^{H} | 01501 | 00601 | 00301 | 01502 | 00601 | 02301 | 1.3 | 5 |
| 14 | Case | ANA^{H} | 01501 | 00601 | 00301 | 01502 | 00601 | 02301 | 1.3 | 5 |
| 15 | Case | ANA^{H} | 01501 | 00601 | 00301 | 01502 | 00601 | 02301 | 1.3 | 5 |
| 16 | Case | ANA^{H} | 01501 | 00601 | 00301 | 01502 | 00601 | 02301 | 1.3 | 5 |
| 17 | Case | ANA^{H} | 01502 | 00601 | 02301 | 02301 | 00301 | 00501 | 1.5 | 6 |
| 18 | Case | ANA^{H} | 00601 | 005011 | 02001 | 00601 | 005011 | 02001 | 2.2 | 2 |
| 19 | Case | ANA^{H} | 00601 | 005011 | 02001 | 00601 | 005011 | 02001 | 2.2 | 2 |
| 20 | Case | ANA^{H} | 00601 | 005011 | 02001 | 01501 | 00601 | 00301 | 2.3 | 7 |
| 21 | Case | ANA^{H} | 01501 | 00601 | 00301 | 01501 | 00601 | 00301 | 3.3 | 3 |
| 22 | Case | ANA^{H} | 01501 | 00601 | 00301 | 01501 | 00601 | 00301 | 3.3 | 3 |
| 23 | Case | ANA^{H} | 01501 | 00601 | 00301 | 01501 | 00601 | 00301 | 3.3 | 3 |
| 24 | Case | ANA^{H} | 01501 | 00601 | 00301 | 01501 | 00601 | 00301 | 3.3 | 3 |
| 25 | Case | ANA^{H} | 01501 | 00601 | 00301 | 01501 | 00601 | 00301 | 3.3 | 3 |
| 26 | Case | ANA^{H} | 01501 | 00601 | 00301 | 01501 | 00601 | 00301 | 3.3 | 3 |
| 27 | Case | ANA^{S} | 00601 | 005011 | 02001 | 01502 | 00601 | 02301 | 1.2 | 4 |
| 28 | Case | ANA^{S} | 00601 | 005011 | 02001 | 01502 | 00601 | 02301 | 1.2 | 4 |
| 29 | Case | ANA^{S} | 00601 | 005011 | 02001 | 01502 | 00601 | 02301 | 1.2 | 4 |
| 30 | Case | ANA^{S} | 02301 | 00301 | 00501 | 01502 | 00601 | 02301 | 1.5 | 6 |
| 31 | Case | ANA^{S} | 00601 | 005011 | 02001 | 00601 | 005011 | 02001 | 2.2 | 2 |
| 32 | Case | ANA^{S} | 00601 | 005011 | 02001 | 00601 | 005011 | 02001 | 2.2 | 2 |
| 33 | Case | ANA^{S} | 00601 | 005011 | 02001 | 00601 | 005011 | 02001 | 2.2 | 2 |
| 34 | Case | ANA^{S} | 00601 | 005011 | 02001 | 00601 | 005011 | 02001 | 2.2 | 2 |
| 35 | Case | ANA^{S} | 00601 | 005011 | 02001 | 00601 | 005011 | 02001 | 2.2 | 2 |
| 36 | Case | ANA^{S} | 00601 | 005011 | 02001 | 00601 | 005011 | 02001 | 2.2 | 2 |
| 37 | Case | ANA^{S} | 00601 | 005011 | 02001 | 00601 | 005011 | 02001 | 2.2 | 2 |
| 38 | Case | ANA^{S} | 00601 | 005011 | 02001 | 00601 | 005011 | 02001 | 2.2 | 2 |
| 39 | Case | ANA^{S} | 00601 | 005011 | 02001 | 00601 | 005011 | 02001 | 2.2 | 2 |
| 40 | Case | ANA^{S} | 00601 | 005011 | 02001 | 00601 | 005011 | 02001 | 2.2 | 2 |
| 41 | Case | ANA^{S} | 00601 | 005011 | 02001 | 00601 | 005011 | 02001 | 2.2 | 2 |
| 42 | Case | ANA^{S} | 00601 | 005011 | 02001 | 00601 | 005011 | 02001 | 2.2 | 2 |
| 43 | Case | ANA^{S} | 00601 | 005011 | 02001 | 00601 | 005011 | 02001 | 2.2 | 2 |
| 44 | Case | ANA^{S} | 00601 | 005011 | 02001 | 00601 | 005011 | 02001 | 2.2 | 2 |
| 45 | Case | ANA^{S} | 00601 | 005011 | 02001 | 00601 | 005011 | 02001 | 2.2 | 2 |
| 46 | Case | ANA^{S} | 00601 | 005011 | 02001 | 00601 | 005011 | 02001 | 2.2 | 2 |
| 47 | Case | ANA^{S} | 00601 | 005011 | 02001 | 00601 | 005011 | 02001 | 2.2 | 2 |
| 48 | Case | ANA^{S} | 00601 | 005011 | 02001 | 00601 | 005011 | 02001 | 2.2 | 2 |
| 49 | Case | ANA^{S} | 00601 | 005011 | 02001 | 00601 | 005011 | 02001 | 2.2 | 2 |
| 50 | Case | ANA^{S} | 00601 | 005011 | 02001 | 00601 | 005011 | 02001 | 2.2 | 2 |
| 51 | Case | ANA^{S} | 00601 | 005011 | 02001 | 00601 | 005011 | 02001 | 2.2 | 2 |
| 52 | Case | ANA^{S} | 00601 | 005011 | 02001 | 00601 | 005011 | 02001 | 2.2 | 2 |
| 53 | Case | ANA^{S} | 00601 | 005011 | 02001 | 00601 | 005011 | 02001 | 2.2 | 2 |
| 54 | Case | ANA^{S} | 00601 | 005011 | 02001 | 00601 | 005011 | 02001 | 2.2 | 2 |
| 55 | Case | ANA^{S} | 00601 | 005011 | 02001 | 00601 | 005011 | 02001 | 2.2 | 2 |
| 56 | Case | ANA^{S} | 00601 | 005011 | 02001 | 00601 | 005011 | 02001 | 2.2 | 2 |
| 57 | Case | ANA^{S} | 02301 | 00301 | 00501 | 00601 | 005011 | 02001 | 2.5 | 8 |
| 58 | Case | ANA^{S} | 01501 | 00601 | 00301 | 01501 | 00601 | 00301 | 3.3 | 3 |
| 59 | Case | Positive | 01502 | 00601 | 02301 | 01502 | 00601 | 02301 | 1.1 | 1 |
| 60 | Case | Positive | 01502 | 00601 | 02301 | 01502 | 00601 | 02301 | 1.1 | 1 |
| 61 | Case | Positive | 00601 | 005011 | 02001 | 01502 | 00601 | 02301 | 1.2 | 4 |
| 62 | Case | Positive | 01501 | 00601 | 00301 | 01502 | 00601 | 02301 | 1.3 | 5 |
| 63 | Case | Positive | 02301 | 00301 | 00501 | 01502 | 00601 | 02301 | 1.5 | 6 |
| 64 | Case | Positive | 00601 | 005011 | 02001 | 00601 | 005011 | 02001 | 2.2 | 2 |
| 65 | Control | Negative | 01502 | 00601 | 02301 | 01502 | 00601 | 02301 | 1.1 | 1 |
| 66 | Control | Negative | 01502 | 00601 | 02301 | 01502 | 00601 | 02301 | 1.1 | 1 |
| 67 | Control | Negative | 01502 | 00601 | 02301 | 01502 | 00601 | 02301 | 1.1 | 1 |
| 68 | Control | Negative | 01502 | 00601 | 02301 | 01502 | 00601 | 02301 | 1.1 | 1 |
| 69 | Control | Negative | 01502 | 00601 | 02301 | 01502 | 00601 | 02301 | 1.1 | 1 |
| 70 | Control | Negative | 01502 | 00601 | 02301 | 01502 | 00601 | 02301 | 1.1 | 1 |
| 71 | Control | Negative | 01502 | 00601 | 02301 | 01502 | 00601 | 02301 | 1.1 | 1 |
| 72 | Control | Negative | 01502 | 00601 | 02301 | 00601 | 005011 | 02001 | 1.2 | 4 |
| 73 | Control | Negative | 01502 | 00601 | 02301 | 00601 | 005011 | 02001 | 1.2 | 4 |
| 74 | Control | Negative | 01502 | 00601 | 02301 | 00601 | 005011 | 02001 | 1.2 | 4 |
| 75 | Control | Negative | 01502 | 00601 | 02301 | 00601 | 005011 | 02001 | 1.2 | 4 |
| 76 | Control | Negative | 01502 | 00601 | 02301 | 00601 | 005011 | 02001 | 1.2 | 4 |
| 77 | Control | Negative | 01502 | 00601 | 02301 | 00601 | 005011 | 02001 | 1.2 | 4 |
| 78 | Control | Negative | 01502 | 00601 | 02301 | 00601 | 005011 | 02001 | 1.2 | 4 |
| 79 | Control | Negative | 01502 | 00601 | 02301 | 00601 | 005011 | 02001 | 1.2 | 4 |
| 80 | Control | Negative | 01502 | 00601 | 02301 | 00601 | 005011 | 02001 | 1.2 | 4 |
| 81 | Control | Negative | 01502 | 00601 | 02301 | 00601 | 005011 | 02001 | 1.2 | 4 |
| 82 | Control | Negative | 01502 | 00601 | 02301 | 00601 | 005011 | 02001 | 1.2 | 4 |
| 83 | Control | Negative | 01502 | 00601 | 02301 | 00601 | 005011 | 02001 | 1.2 | 4 |
| 84 | Control | Negative | 01502 | 00601 | 02301 | 00601 | 005011 | 02001 | 1.2 | 4 |
| 85 | Control | Negative | 01502 | 00601 | 02301 | 00601 | 005011 | 02001 | 1.2 | 4 |
| 86 | Control | Negative | 01502 | 00601 | 02301 | 00601 | 005011 | 02001 | 1.2 | 4 |
| 87 | Control | Negative | 01502 | 00601 | 02301 | 00601 | 005011 | 02001 | 1.2 | 4 |
| 88 | Control | Negative | 01502 | 00601 | 02301 | 00601 | 005011 | 02001 | 1.2 | 4 |
| 89 | Control | Negative | 01502 | 00601 | 02301 | 00601 | 005011 | 02001 | 1.2 | 4 |
| 90 | Control | Negative | 01502 | 00601 | 02301 | 00601 | 005011 | 02001 | 1.2 | 4 |
| 91 | Control | Negative | 01502 | 00601 | 02301 | 00601 | 005011 | 02001 | 1.2 | 4 |
| 92 | Control | Negative | 01502 | 00601 | 02301 | 00601 | 005011 | 02001 | 1.2 | 4 |
| 93 | Control | Negative | 01502 | 00601 | 02301 | 00601 | 005011 | 02001 | 1.2 | 4 |
| 94 | Control | Negative | 01502 | 00601 | 02301 | 01501 | 00601 | 00301 | 1.3 | 5 |
| 95 | Control | Negative | 01502 | 00601 | 02301 | 01501 | 00601 | 00301 | 1.3 | 5 |
| 96 | Control | Negative | 01502 | 00601 | 02301 | 01501 | 00601 | 00301 | 1.3 | 5 |
| 97 | Control | Negative | 01502 | 00601 | 02301 | 01501 | 00601 | 00301 | 1.3 | 5 |
| 98 | Control | Negative | 01502 | 00601 | 02301 | 01501 | 00601 | 00301 | 1.3 | 5 |
| 99 | Control | Negative | 01502 | 00601 | 02301 | 01501 | 00601 | 00301 | 1.3 | 5 |
| 100 | Control | Negative | 01502 | 00601 | 02301 | 01501 | 00601 | 00301 | 1.3 | 5 |
| 101 | Control | Negative | 01502 | 00601 | 02301 | 01501 | 00601 | 00301 | 1.3 | 5 |
| 102 | Control | Negative | 01502 | 00601 | 02301 | 01501 | 00601 | 00301 | 1.3 | 5 |
| 103 | Control | Negative | 01502 | 00601 | 02301 | 01501 | 00601 | 00301 | 1.3 | 5 |
| 104 | Control | Negative | 01502 | 00601 | 02301 | 01501 | 00601 | 00301 | 1.3 | 5 |
| 105 | Control | Negative | 01502 | 00601 | 02301 | 01501 | 00601 | 00301 | 1.3 | 5 |
| 106 | Control | Negative | 01502 | 00601 | 02301 | 00401 | 00201 | 01501 | 1.4 | 10 |
| 107 | Control | Negative | 01502 | 00601 | 02301 | 02301 | 00301 | 00501 | 1.5 | 6 |
| 108 | Control | Negative | 01502 | 00601 | 02301 | 02301 | 00301 | 00501 | 1.5 | 6 |
| 109 | Control | Negative | 01502 | 00601 | 02301 | 02301 | 00301 | 00501 | 1.5 | 6 |
| 110 | Control | Negative | 01502 | 00601 | 02301 | 02301 | 00301 | 00501 | 1.5 | 6 |
| 111 | Control | Negative | 01502 | 00601 | 02301 | 02301 | 00301 | 00501 | 1.5 | 6 |
| 112 | Control | Negative | 01502 | 00601 | 02301 | 02301 | 00301 | 00501 | 1.5 | 6 |
| 113 | Control | Negative | 00601 | 005011 | 02001 | 00601 | 005011 | 02001 | 2.2 | 2 |
| 114 | Control | Negative | 00601 | 005011 | 02001 | 00601 | 005011 | 02001 | 2.2 | 2 |
| 115 | Control | Negative | 00601 | 005011 | 02001 | 00601 | 005011 | 02001 | 2.2 | 2 |
| 116 | Control | Negative | 00601 | 005011 | 02001 | 00601 | 005011 | 02001 | 2.2 | 2 |
| 117 | Control | Negative | 00601 | 005011 | 02001 | 00601 | 005011 | 02001 | 2.2 | 2 |
| 118 | Control | Negative | 00601 | 005011 | 02001 | 00601 | 005011 | 02001 | 2.2 | 2 |
| 119 | Control | Negative | 00601 | 005011 | 02001 | 00601 | 005011 | 02001 | 2.2 | 2 |
| 120 | Control | Negative | 00601 | 005011 | 02001 | 00601 | 005011 | 02001 | 2.2 | 2 |
| 121 | Control | Negative | 00601 | 005011 | 02001 | 00601 | 005011 | 02001 | 2.2 | 2 |
| 122 | Control | Negative | 00601 | 005011 | 02001 | 01501 | 00601 | 00301 | 2.3 | 7 |
| 123 | Control | Negative | 00601 | 005011 | 02001 | 01501 | 00601 | 00301 | 2.3 | 7 |
| 124 | Control | Negative | 00601 | 005011 | 02001 | 01501 | 00601 | 00301 | 2.3 | 7 |
| 125 | Control | Negative | 00601 | 005011 | 02001 | 01501 | 00601 | 00301 | 2.3 | 7 |
| 126 | Control | Negative | 00601 | 005011 | 02001 | 01501 | 00601 | 00301 | 2.3 | 7 |
| 127 | Control | Negative | 00601 | 005011 | 02001 | 01501 | 00601 | 00301 | 2.3 | 7 |
| 128 | Control | Negative | 00601 | 005011 | 02001 | 01501 | 00601 | 00301 | 2.3 | 7 |
| 129 | Control | Negative | 00601 | 005011 | 02001 | 01501 | 00601 | 00301 | 2.3 | 7 |
| 130 | Control | Negative | 00601 | 005011 | 02001 | 01501 | 00601 | 00301 | 2.3 | 7 |
| 131 | Control | Negative | 00601 | 005011 | 02001 | 02301 | 00301 | 00501 | 2.5 | 8 |
| 132 | Control | Negative | 00601 | 005011 | 02001 | 02301 | 00301 | 00501 | 2.5 | 8 |
| 133 | Control | Negative | 00601 | 005011 | 02001 | 02301 | 00301 | 00501 | 2.5 | 8 |
| 134 | Control | Negative | 00601 | 005011 | 02001 | 02301 | 00301 | 00501 | 2.5 | 8 |
| 135 | Control | Negative | 01501 | 00601 | 00301 | 01501 | 00601 | 00301 | 3.3 | 3 |
| 136 | Control | Negative | 01501 | 00601 | 00301 | 01501 | 00601 | 00301 | 3.3 | 3 |
| 137 | Control | Negative | 01501 | 00601 | 00301 | 02301 | 00301 | 00501 | 3.5 | 9 |
| 138 | Control | Negative | 01501 | 00601 | 00301 | 02301 | 00301 | 00501 | 3.5 | 9 |
| 139 | Control | Negative | 01501 | 00601 | 00301 | 02301 | 00301 | 00501 | 3.5 | 9 |
| 140 | Control | Negative | 01501 | 00601 | 00301 | 02301 | 00301 | 00501 | 3.5 | 9 |
| 141 | Control | Negative | 01501 | 00601 | 00301 | 02301 | 00301 | 00501 | 3.5 | 9 |
| 142 | Control | Negative | 00401 | 00201 | 01501 | 02301 | 00301 | 00501 | 4.5 | 11 |

^{S}Speckled,
^{H}Homogeneous

A total of five DLA-DRB1, four DLA-DQA1 and five DLA-DQB1 alleles, forming five different haplotypes were identified (Tables 4 and 5). Eleven different genotypes were observed in the study population (Table 6). Association analysis was performed for alleles, haplotypes and genotypes for the ANA^{H} and ANA^{S} case groups separately as well as the combined case group, and each compared to controls (Tables 7 and 8). There was a significant association with haplotype 2 in all cases compared to the control group (p=0.0006), and a more significant association to homozygosity for this haplotype (genotype 2; 45.3% in cases vs. 11.5% in controls; OR=6.4 and p<0.001). Still, the strongest association was seen for genotype 2 to the ANA^{S} phenotype. In fact, thirty of the thirty-two ANA^{S} dogs were either homo- or heterozygous for haplotype 2. Twenty-six ANA^{S} dogs (81.3%) were homozygous for haplotype 2 (DLA-DRBI*00601/DQA1*005011/DQB1*02001) compared to 9 (11.5%) of the controls, OR=33.2 and p<0.0001. No significant association was observed between the haplotypes or genotypes of MHC class II and the cases with ANA^{H} pattern, but when removing the ANA^{S} risk genotype and analyzing the remaining data (Table 9), an increase in homozygosity of any haplotype was seen in ANA^{H} cases 62.5% vs. controls 13.0% (OR=11.1 and p<0.0001), implicating a general homozygous disadvantage at DLA class II for ANA^{H} dogs (Table 10).

TABLE 4
Allele frequencies in NSDTR population (all ANA-positive cases,
ANA^{S}, ANA^{H} and controls) identified five different DRB1,
four DQA1 and five DQB1 alleles.

| | Tot pop | Controls | All cases | ANA^{S} | ANA^{H} |
|---|---|---|---|---|---|
| | % | % | % | % | % |
| Alleles | 2N = 284 | 2N = 156 | 2N = 128 | 2N = 64 | 2N = 52 |
| DRB1*00401 | 0.7 (2) | 1.3 (2) | 0.0 (0) | 0.0 (0) | 0.0 (0) |
| DRB1*00601 | 41.5 (118) | 34.0 (53) | 50.8 (65) | 87.5 (56) | 11.5 (6) |
| DRB1*01501 | 18.3 (52) | 19.2 (30) | 17.2 (22) | 3.1 (2) | 36.5 (19) |
| DRB1*01502 | 32.4 (92) | 35.3 (55) | 28.9 (37) | 6.3 (4) | 50.0 (26) |
| DRB1*02301 | 7.0 (20) | 10.3 (16) | 3.1 (4) | 3.1 (2) | 1.9 (1) |
| DQA1*00601 | 50.7 (144) | 54.5 (85) | 46.1 (59) | 9.4 (6) | 86.5 (45) |
| DQA1*005011 | 41.5 (118) | 34.0 (53) | 50.8 (65) | 87.5 (56) | 11.5 (6) |
| DQA1*00201 | 0.7 (2) | 1.3 (2) | 0.0 (0) | 0.0 (0) | 0.0 (0) |
| DQA1*00301 | 7.0 (20) | 10.3 (16) | 3.1 (4) | 3.1 (2) | 1.9 (1) |
| DQB1*02301 | 32.4 (92) | 35.3 (55) | 28.9 (37) | 6.3 (4) | 50.0 (26) |
| DQB1*02001 | 41.5 (118) | 34.0 (53) | 50.8 (65) | 87.5 (56) | 11.5 (6) |
| DQB1*00301 | 18.3 (52) | 19.2 (30) | 17.2 (22) | 3.1 (2) | 36.5 (19) |
| DQB1*01501 | 0.7 (2) | 1.3 (2) | 0.0 (0) | 0.0 (0) | 0.0 (0) |
| DQB1*00501 | 7.0 (20) | 10.3 (16) | 3.1 (4) | 3.1 (2) | 1.9 (1) |

^{S} = Speckled,
^{H} = Homogeneous
Parenthesis indicates number of alleles

TABLE 5
Haplotype frequencies in the NSDTR population reveal an associated haplotype for
ANA^{S} dogs (DLA-DRB1*00601/DQA1*005011/DQB1*02001).

| Haplotype | Haplotype | Tot pop % | Controls % | All cases % | ANA^{S} % | ANA^{H} % |
|---|---|---|---|---|---|---|
| No | DRB1/DQA1/DQB1 | 2N = 284 | 2N = 156 | 2N = 128 | 2N = 64 | 2N = 52 |
| 1 | 01502/00601/02301 | 32.4 (92) | 35.3 (55) | 28.9 (37) | 6.3 (4) | 50.0 (26) |
| 2 | 00601/005011/02001 | 41.5 (118) | 34.0 (53) | 50.8 (65) | 87.5 (56) | 11.5 (6) |
| 3 | 01501/00601/00301 | 18.3 (52) | 19.2 (30) | 17.2 (22) | 3.1 (2) | 36.5 (19) |
| 4 | 00401/00201/01501 | 0.7 (2) | 1.3 (2) | 0.0 (0) | 0.0 (0) | 0.0 (0) |
| 5 | 02301/00301/00501 | 7.0 (20) | 10.3 (16) | 3.1 (4) | 3.1 (2) | 1.9 (1) |

^{S}Speckled,
^{H}Homogeneous
The haplotype associated with the speckled ANA phenotype is indicated in bold
Parenthesis indicates number of alleles

TABLE 6
Genotype frequencies in NSDTR population indicate an increased frequency for
ANA^{S} dogs homozygous for haplotype 2 (DLA-DRBI*00601/DQA1*005011/DQB1*02001)
compared to controls.

| Genotype | | Tot pop % | Controls % | All cases % | ANA^{S} % | ANA^{H} % |
|---|---|---|---|---|---|---|
| No | Haplotypes | N = 142 | N = 78 | N = 64 | N = 32 | N = 26 |
| 1 | 1.1 | 12.7 (18) | 9.0 (7) | 17.2 (11) | 0.0 (0) | 34.6 (9) |
| 2 | 2.2 | 26.8 (38) | 11.5 (9) | 45.3 (29) | 81.3 (26) | 7.7 (2) |
| 3 | 3.3 | 6.3 (9) | 2.6 (2) | 10.9 (7) | 3.1 (1) | 23.1 (6) |
| 4 | 1.2 | 19.0 (27) | 28.2 (22) | 7.8 (5) | 9.4 (3) | 3.8 (1) |
| 5 | 1.3 | 13.4 (19) | 15.4 (12) | 10.9 (7) | 0.0 (0) | 23.1 (6) |
| 6 | 1.5 | 6.3 (9) | 7.7 (6) | 4.7 (3) | 3.1 (1) | 3.8 (1) |
| 7 | 2.3 | 7.0 (10) | 11.5 (9) | 1.6 (1) | 0.0 (0) | 3.8 (1) |
| 8 | 2.5 | 3.5 (5) | 5.1 (4) | 1.6 (1) | 3.1 (1) | 0.0 (0) |
| 9 | 3.5 | 3.5 (5) | 6.4 (5) | 0.0 (0) | 0.0 (0) | 0.0 (0) |
| 10 | 1.4 | 0.7 (1) | 1.3 (1) | 0.0 (0) | 0.0 (0) | 0.0 (0) |
| 11 | 4.5 | 0.7 (1) | 1.3 (1) | 0.0 (0) | 0.0 (0) | 0.0 (0) |

^{S}Speckled,
^{H}Homogeneous
Genotypes associated with ANA^{S} are in bold
Parentheses indicate number of dogs

TABLE 7
MHC class II haplotype frequencies
observed in the NSDTR population.

| | Controls % | Odds ratio | |
|---|---|---|---|
| Disease % affected | 2N = 156 | (95% CI^{a}) | P value |
| Haplotype 1 | | | |
| All cases 2N = 128 | | | |
| 28.9 (37) | 35.3 (55) | 0.75 (0.45-1.23) | 0.31 |
| ANA^{H} 2N = 52 | | | |
| 50.0 (26) | 35.3 (55) | 1.8 (0.97-3.45) | 0.08 |
| ANA^{S} 2N = 64 | | | |
| 6.3 (4) | 35.3 (55) | 0.12 (0.04-0.35) | <.0001 |
| Haplotype 2 | | | |
| All cases 2N = 128 | | | |
| 50.8 (65) | 34.0 (53) | 2.0 (1.2-3.2) | 0.006 |
| ANA^{H} 2N = 52 | | | |
| 11.5 (6) | 34.0 (53) | 0.25 (0.10-0.63) | 0.003 |
| ANA^{S} 2N = 64 | | | |
| 87.5 (56) | 34.0 (53) | 13.6 (6.0-30.6) | <0001 |
| Haplotype 3 | | | |
| All cases 2N = 128 | | | |
| 17.2 (22) | 19.2 (30) | 0.87 (0.47-1.6) | 0.8 |
| ANA^{H} 2N = 52 | | | |
| 36.5 (19) | 19.2 (30) | 2.4 (1.2-4.8) | 0.02 |
| ANA^{S} 2N = 64 | | | |
| 3.1 (2) | 19.2 (30) | 0.14 (0.03-0.59) | 0.004 |
| Haplotype 4 | | | |
| All cases 2N = 128 | | | |
| 0.0 (0) | 1.3 (2) | 0 | — |
| ANA^{H} 2N = 52 | | | |
| 0.0 (0) | 1.3 (2) | 0 | — |
| ANA^{S} 2N = 64 | | | |
| 0.0 (0) | 1.3 (2) | 0 | — |
| Haplotype 5 | | | |
| All cases 2N = 128 | | | |
| 3.1 (4) | 10.3 (16) | 0.28 (0.09-0.87) | 0.04 |
| ANA^{H} 2N = 52 | | | |
| 1.9 (1) | 10.3 (16) | 0.17 (0.02-1.33) | — |
| ANA^{S} 2N = 64 | | | |
| 3.1 (2) | 10.3 (16) | 0.28 (0.06-1.27) | 0.14 |

^{S} = Speckled,
^{H} = Homogeneous
^{a}CI = Confidence Interval
Parenthesis indicates number of alleles

TABLE 8
MHC class II genotype frequencies observed in the NSDTR population.

| Disease % affected | Controls % N = 78 | Odds ratio (95% CIa) | P value |
|---|---|---|---|
| Genotype 1 | | | |
| All cases N = 64 | | | |
| 17.2 (11) | 9.0 (7) | 2.1 (0.77-5.8) | 0.23 |
| ANA^{H} N = 26 | | | |
| 34.6 (9) | 9.0 (7) | 5.4 (1.8-16.5) | — |
| ANA^{S} N = 32 | | | |
| 0.0 (0) | 9.0 (7) | Infinity (NaN-Infinity) | <.0001 |
| Genotype 2 | | | |
| All cases N = 64 | | | |
| 45.3 (29) | 11.5 (9) | 6.4 (2.7-14.9) | <.0001 |
| ANA^{H} N = 26 | | | |
| 7.7 (2) | 11.5 (9) | 0.64 (0.13-3.2) | — |
| ANA^{S} N = 32 | | | |
| 81.3 (26) | 11.5 (9) | 33.2 (10.8-102.6) | <.0001 |
| Genotype 3 | | | |
| All cases N = 64 | | | |
| 10.9 (7) | 2.6 (2) | 4.7 (0.93-23.3) | — |
| ANA^{H} N = 26 | | | |
| 23.1 (6) | 2.6 (2) | 11.4 (2.1-60.8) | — |
| ANA^{S} N = 32 | | | |
| 3.1 (1) | 2.6 (2) | 1.2 (0.11-14.0) | — |
| Genotype 4 | | | |
| All cases N = 64 | | | |
| 6.3 (4) | 28.2 (22) | 0.17 (0.06-0.52) | 0.002 |
| ANA^{H} N = 26 | | | |
| 0.0 (0) | 28.2 (22) | 0 | 0.006 |
| ANA^{S} N = 32 | | | |
| 9.4 (3) | 28.2 (22) | 0.26 (0.07-0.95) | 0.06 |
| Genotype 5 | | | |
| All cases N = 64 | | | |
| 12.5 (8) | 15.4 (12) | 0.79 (0.3-2.1) | 0.81 |
| ANA^{H} N = 26 | | | |
| 26.9 (7) | 15.4 (12) | 2.0 (0.70-5.9) | — |
| ANA^{S} N = 32 | | | |
| 0.0 (0) | 15.4 (12) | 0 | — |
| Genotype 6 | | | |
| All cases N = 64 | | | |
| 4.7 (3) | 7.7 (6) | 0.59 (0.14-2.5) | — |
| ANA^{H} N = 26 | | | |
| 3.8 (1) | 7.7 (6) | 0.48 (0.06-4.2) | — |
| ANA^{S} N = 32 | | | |
| 3.1 (1) | 7.7 (6) | 0.39 (0.05-3.4) | — |
| Genotype 7 | | | |
| All cases N = 64 | | | |
| 1.6 (1) | 11.5 (9) | 0.12 (0.02-0.99) | — |
| ANA^{H} N = 26 | | | |
| 3.8 (1) | 11.5 (9) | 0.31 (0.04-2.5) | — |
| ANA^{S} N = 32 | | | |
| 0.0 (0) | 11.5 (9) | 0 | — |
| Genotype 8 | | | |
| All cases N = 64 | | | |
| 1.6 (1) | 5.1 (4) | 0.29 (0.03-2.7) | — |
| ANA^{H} N = 26 | | | |
| 0.0 (0) | 5.1 (4) | 0 | — |
| ANA^{S} N = 32 | | | |
| 3.1 (1) | 5.1 (4) | 0.60 (0.06-5.6) | — |
| Genotype 9 | | | |
| All cases N = 64 | | | |
| 0.0 (0) | 6.4 (5) | 0 | — |
| ANA^{H} N = 26 | | | |
| 0.0 (0) | 6.4 (5) | 0 | — |
| ANA^{S} N = 32 | | | |
| 0.0 (0) | 6.4 (5) | 0 | — |
| Genotype 10 | | | |
| All cases N = 64 | | | |
| 0.0 (0) | 1.3 (1) | 0 | — |
| ANA^{H} N = 26 | | | |
| 0.0 (0) | 1.3 (1) | 0 | — |
| ANA^{S} N = 32 | | | |
| 0.0 (0) | 1.3 (1) | 0 | — |
| Genotype 11 | | | |
| All cases N = 64 | | | |
| 0.0 (0) | 1.3 (1) | 0 | — |
| ANA^{H} N = 26 | | | |
| 0.0 (0) | 1.3 (1) | 0 | — |
| ANA^{S} N = 32 | | | |
| 0.0 (0) | 1.3 (1) | 0 | — |

^{a}CI = Confidence Interval
Parenthesis indicates number of dogs

TABLE 9
Homozygosity at MHC class II observed in the NSDTR population.

| | Tot pop % | Controls % | All cases % | ANA^{S} % | ANA^{H} % |
|---|---|---|---|---|---|
| | N = 142 | N = 78 | N = 64 | N = 32 | N = 26 |
| Homozygous | 45.8 (65) | 23.1 (18) | 73.4 (47) | 84.4 (27) | 65.4 (17) |
| | Tot pop % | Controls % | All cases % | ANA^{S} % | ANA^{H} % |
| | N = 104 | N = 69 | N = 35 | N = 6 | N = 24 |
| Homozygous | 26.0 (27) | 13.0 (9) | 51.4 (18) | 16.7 (1) | 62.5 (15) |
| no risk | | | | | |

^{S} = Speckled,
^{H} = Homogeneous
Parenthesis indicates number of dogs

TABLE 10
Homozygosity at MHC class II observed in the NSDTR population
including all genotypes with and without the risk genotype 2.
Homozygosity observed in the NSDTR population.

| | Controls % | | |
|---|---|---|---|
| Disease % affected | N = 78 | Odds ratio (95% CI^{a}) | P value |
| All cases n = 64 | | | |
| 73.4 (47) | 23.1 (18) | 9.2 (4.3-19.8) | <.0001 |
| ANA^{H} n = 26 | | | |
| 65.4 (17) | 23.1 (18) | 6.3 (2.4-16.5) | 0.0002 |
| ANA^{S} n = 32 | | | |
| 84.4 (27) | 23.1 (18) | 18.0 (6.1-53.5) | <.0001 |
| | Controls % | | |
| Homozygosity no risk | N = 69 | Odds ratio (95% CI^{a}) | P value |
| All cases n = 35 | | | |
| 51.4 (18) | 13.0 (9) | 7.1 (2.7-18.5) | <.0001 |
| ANA^{H} n = 24 | | | |
| 62.5 (15) | 13.0 (9) | 11.1 (3.8-32.8) | <.0001 |
| ANA^{S} n = 6 | | | |
| 16.7 (1) | 13.0 (9) | 1.3 (0.14-12.8) | — |

^{a}CI = Confidence Interval
Parenthesis indicates number of dogs

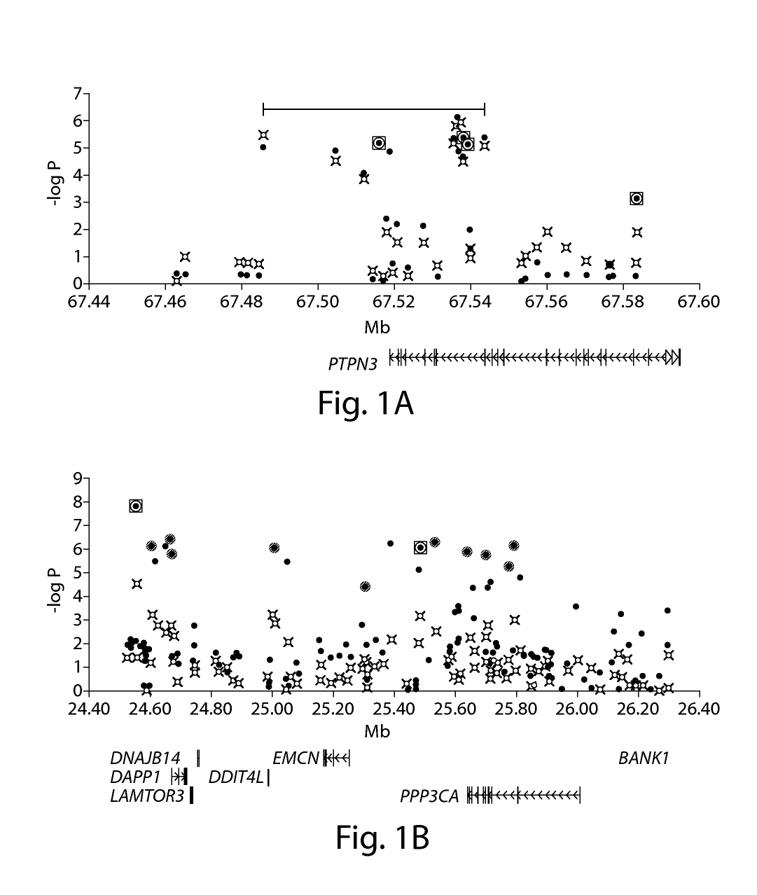

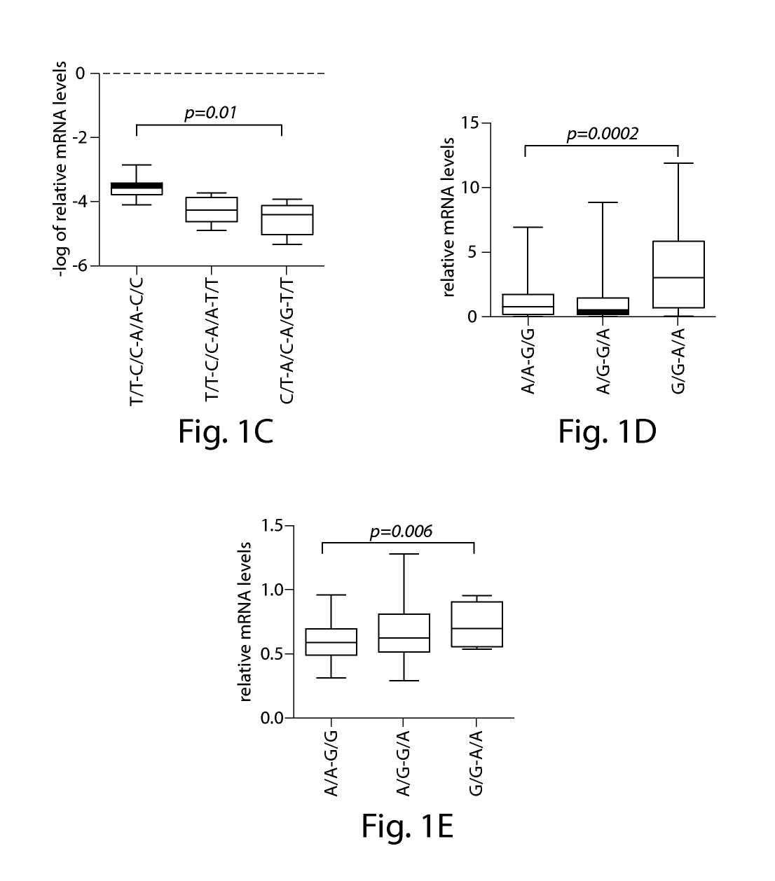

Next, genome wide association (GWA) risk loci on CFA 3, 8, 11, 24 and 322 were examined for association to the speckled phenotype (ANA^{S}). To search for candidate variants, we re-sequenced five associated regions [ref. 2] in four ANA-positive cases and three healthy dogs using Nimblegen capture and Illumina sequencing. 305 SNPs fitting the risk haplotype pattern were chosen for genotyping in the entire data set. A conditional analysis was performed where only dogs homozygous for DLA risk haplotype 2 were included (N=25) and 145 controls. Strong associations to chromosome 11 and 32 were observed in ANA^{S} dogs that were homozygous for DLA haplotype 2. None of the other chromosomal regions showed a significant association in the conditional analysis. On chromosome 11, a 15 SNP haplotype covering the PTPN3 gene was highly associated to the ANA^{S} phenotype (p=8.2×10⁻⁷, OR=5.7) (Table 11). The most associated region on chromosome 32 (p=1.5×10-8-4.7×10⁻⁵ and OR=3.4-6.8) was 1.3 Mb in size and contained DAPP1, LAMTOR3, DNAJB14, H2AFZ, DDIT4L, EMCN, PPP3CA and BANK1 genes (Table 12). The regions on chromosomes 11 and 32 (FIG. 1) were also examined for association in the larger cohort containing all ANA-positive dogs (N=63, consisting of 30 ANA^{S}, 22 ANA^{H} and 11 non-classified dogs). Similar levels of association were observed to the chromosome 11 region for both all ANA+ dogs together (p=1.8×10⁻⁶) and the ANA^{S} dogs alone (p=8.2×10⁻⁷), implying that this region is more strongly associated with ANA^{S} as fewer dogs gave a similar association. On chromosome 32, the ANAS group (p=1.5×10⁻⁸) gave a 1,000-fold stronger association compared to all ANA+ dogs (p=3.1×10⁻⁵). A similar result was obtained in a non-conditional analysis, where all speckled dogs (N=30) were used regardless of MHC haplotype.

TABLE 11
Association on chromosome 11 in ANA^{S} dogs with genotype 2 for MHC class II.

| Position | A1 | F_A | F_U | A2 | P^{raw} | OR | Location | Conservation ^{a} |
|---|---|---|---|---|---|---|---|---|
| chr11:67536642 | C | 0.35 | 0.09 | A | 8.19E−07 | 5.7 | Intron | NC |
| chr11:67535953 | A | 0.31 | 0.08 | G | 4.45E−06 | 5 | Intron | NC |
| chr11:67543652 | A | 0.31 | 0.08 | G | 4.45E−06 | 5 | Intron | NC |
| chr11:67538032 | A | 0.31 | 0.08 | C | 4.63E−06 | 5.0 | Intron | C |
| chr11:67516041 | C | 0.31 | 0.09 | T | 6.93E−06 | 4.8 | 3′ UTR | C |
| chr11:67537177 | G | 0.31 | 0.09 | A | 6.93E−06 | 4.8 | Intron | NC |
| chr11:67537363 | A | 0.31 | 0.09 | G | 6.93E−06 | 4.8 | Intron | NC |
| chr11:67538806 | G | 0.31 | 0.09 | A | 6.93E−06 | 4.8 | Synonymous | C |
| chr11:67537493 | A | 0.31 | 0.09 | G | 8.81E−06 | 4.74 | Intron | C |
| chr11:67485866 | A | 0.30 | 0.08 | G | 9.58E−06 | 4.7 | Downstream | C |
| chr11:67504858 | A | 0.30 | 0.09 | G | 1.47E−05 | 4.5 | Downstream | NC |
| chr11:67518596 | A | 0.30 | 0.09 | G | 1.47E−05 | 4.5 | Intron | NC |
| chr11:67518781 | A | 0.30 | 0.09 | G | 1.47E−05 | 4.5 | Intron | C |
| chr11:67536944 | A | 0.30 | 0.09 | G | 1.47E−05 | 4.5 | Intron | C |
| chr11:67537924 | A | 0.30 | 0.089 | C | 2.19E−05 | 4.5 | Intron | NC |
| chr11:67511882 | C | 0.42 | 0.17 | A | 8.84E−05 | 3.5 | Downstream | NC |
| chr11:67583604 | T | 0.58 | 0.331 | C | 7.46E−04 | 2.8 | Synonymous | C |

^{a} NC = Not conserved, C = Conserved. Based on UCSC PhastCons Conserved Elements.
The differences observed in allele frequencies are according to differences in genotyping rates.

TABLE 12
Association on chromosome 32 in ANA^{S} dogs with genotype 2 for MHC class II.

| Position | A1 | F_A | F_U | A2 | P^{raw} | OR | Location | Conservation ^{a} |
|---|---|---|---|---|---|---|---|---|
| chr32:24556037 | G | 0.50 | 0.15 | A | 1.50E−08 | 5.7 | Upstream DAPP1 | NC |
| chr32:24667283 | A | 0.26 | 0.05 | G | 4.09E−07 | 6.8 | Upstream DAPP1 | C |
| chr32:25537276 | G | 0.58 | 0.23 | A | 5.58E−07 | 4.7 | Downstream PPP3CA | C |
| chr32:25392401 | A | 0.59 | 0.23 | G | 6.04E−07 | 4.7 | Downstream PPP3CA | C |
| chr32:24606503 | A | 0.27 | 0.06 | G | 7.75E−07 | 6.4 | Upstream DAPP1 | NC |
| chr32:24650093 | A | 0.27 | 0.056 | G | 7.75E−07 | 6.4 | Upstream DAPP1 | C |
| chr32:25798353 | G | 0.66 | 0.30 | A | 7.86E−07 | 4.6 | Intron PPP3CA | C |
| chr32:25485961 | A | 0.50 | 0.18 | G | 9.11E−07 | 4.6 | Downstream PPP3CA | C |
| chr32:25007496 | G | 0.50 | 0.18 | A | 9.83E−07 | 4.4 | Upstream DDIT4L | C |
| chr32:25007632 | C | 0.50 | 0.18 | A | 9.83E−07 | 4.4 | Upstream DDIT4L | C |
| chr32:25642357 | T | 0.28 | 0.06 | A | 1.41E−06 | 5.8 | Intron PPP3CA | C |
| chr32:25485644 | A | 0.48 | 0.18 | G | 1.67E−06 | 4.3 | Downstream PPP3CA | C |
| chr32:24672221 | A | 0.26 | 0.06 | G | 1.68E−06 | 6.0 | Upstream DAPP1 | C |
| chr32:25702963 | G | 0.27 | 0.06 | A | 1.90E−06 | 5.9 | Intron PPP3CA | C |
| chr32:24618331 | C | 0.26 | 0.06 | G | 3.54E−06 | 5.6 | Upstream DAPP1 | C |
| chr32:25049586 | A | 0.26 | 0.06 | G | 3.54E−06 | 5.6 | Upstream DDIT4L | C |
| chr32:25779083 | C | 0.30 | 0.08 | A | 6.10E−06 | 4.9 | Intron PPP3CA | C |
| chr32:25484844 | A | 0.62 | 0.29 | G | 7.83E−06 | 3.9 | Downstream PPP3CA | NC |
| chr32:25816401 | A | 0.65 | 0.32 | G | 1.68E−05 | 3.9 | Intron PPP3CA | C |
| chr32:25718852 | G | 0.40 | 0.15 | A | 2.65E−05 | 3.8 | Intron PPP3CA | NC |
| chr32:25305524 | G | 0.28 | 0.08 | A | 4.35E−05 | 4.31 | Upstream EMCN | C |
| chr32:25710678 | C | 0.54 | 0.26 | A | 4.69E−05 | 3.4 | Intron PPP3CA | C |
| chr32:25662984 | A | 0.63 | 0.32 | G | 4.73E−05 | 3.6 | Intron PPP3CA | C |

^{a} NC = Not conserved, C = Conserved. Based on UCSC PhastCons Conserved Elements.

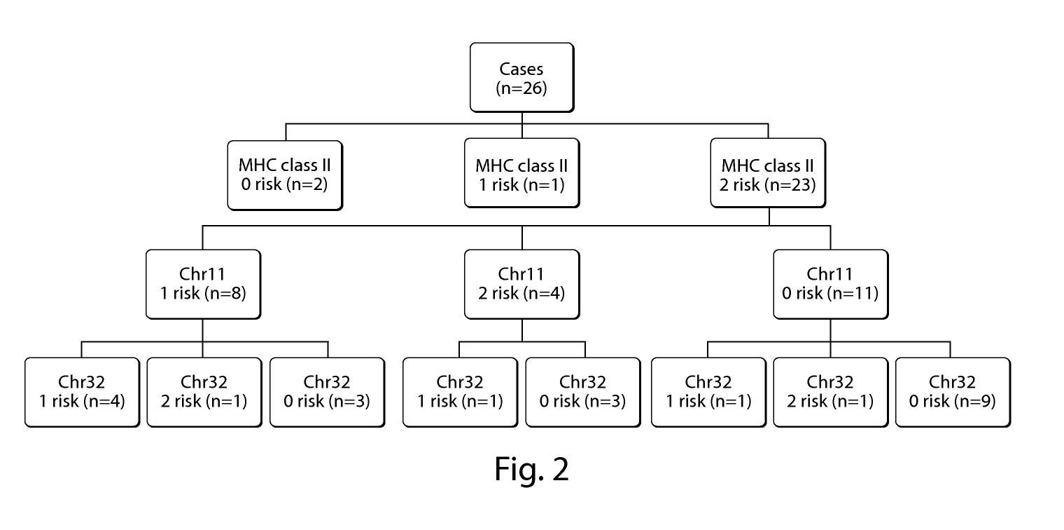

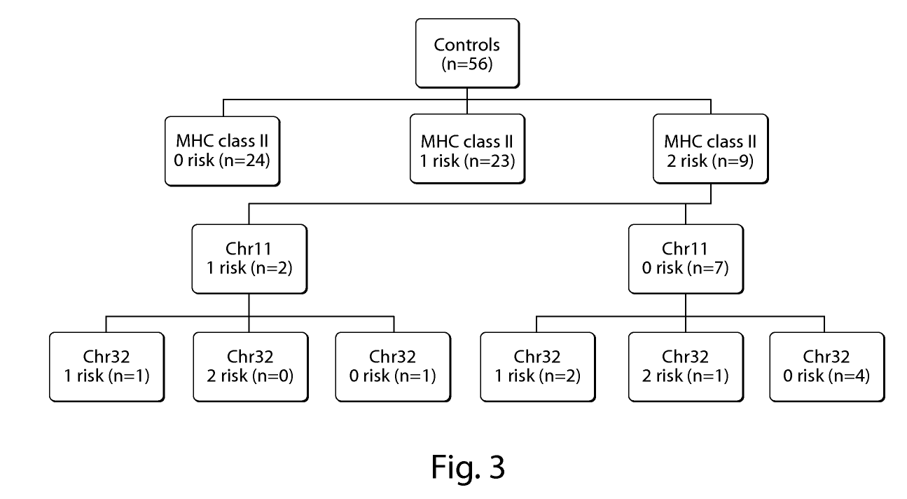

A multi-locus analysis was performed in all dogs with genotypes for all the three candidate loci (26 ANA^{S} cases and 56 healthy controls). The results indicate a larger number of risk alleles for the loci on chromosomes 11 and 32 among the ANA^{S} homozygous cases (28%; FIG. 2) than among the controls that were homozygous for the MHC risk haplotype (19.4%; FIG. 3). Although the number of dogs is too small for performing a formal analysis of epistasis, the results indicate that these three loci jointly contribute to the disease risk and suggest that prediction of disease risk is likely to improve by considering the multi-locus genotype of the individual rather than focusing entirely on the MHC class II.

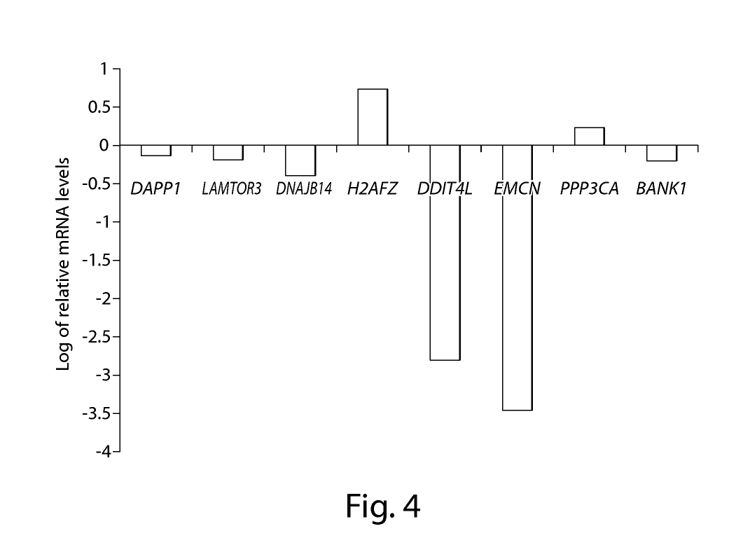

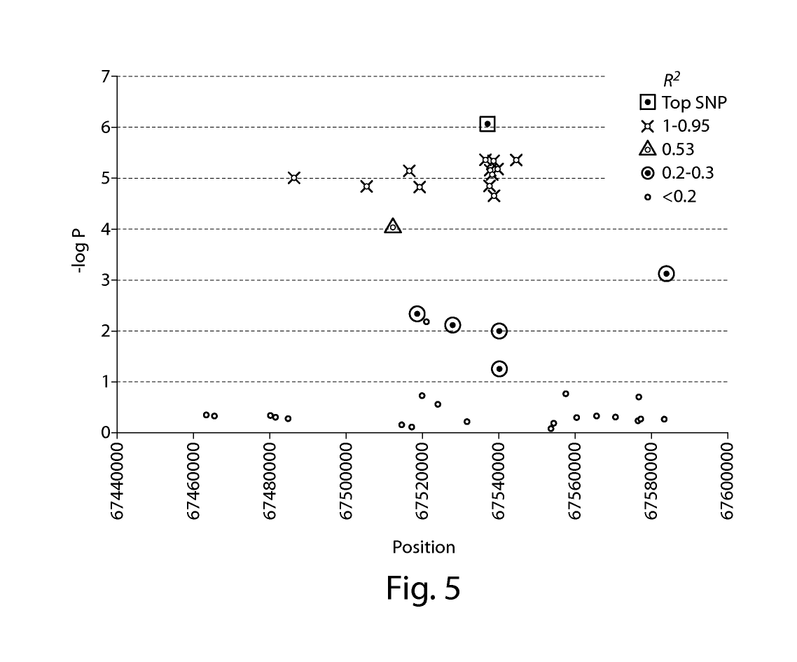

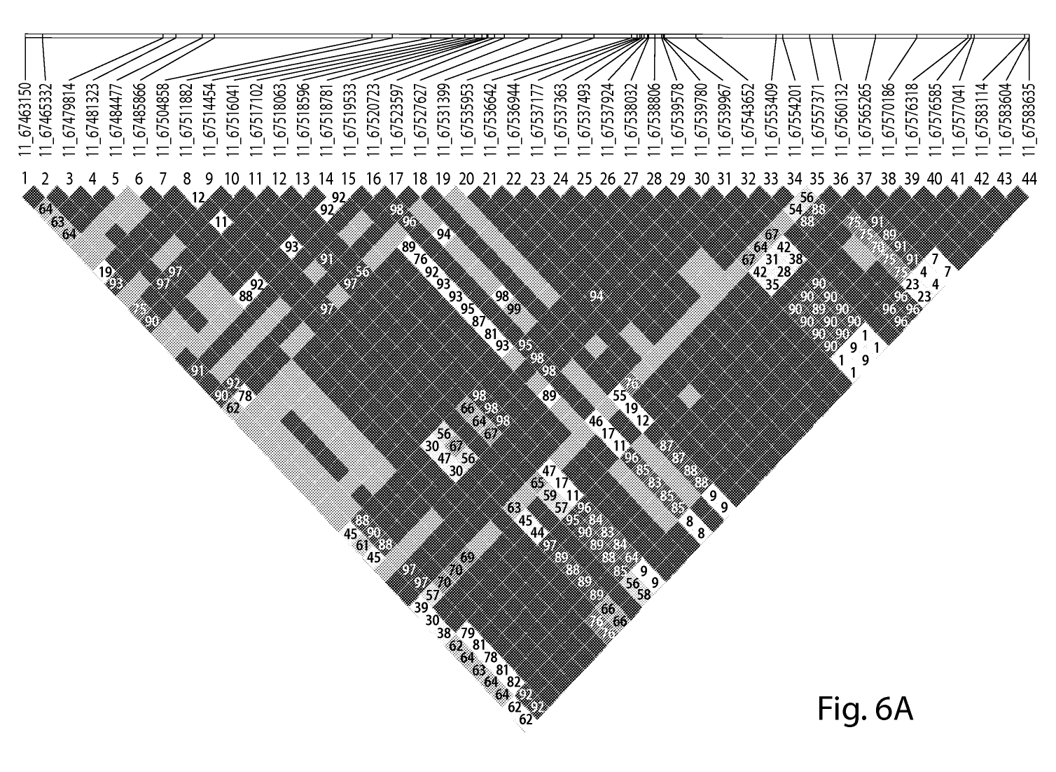

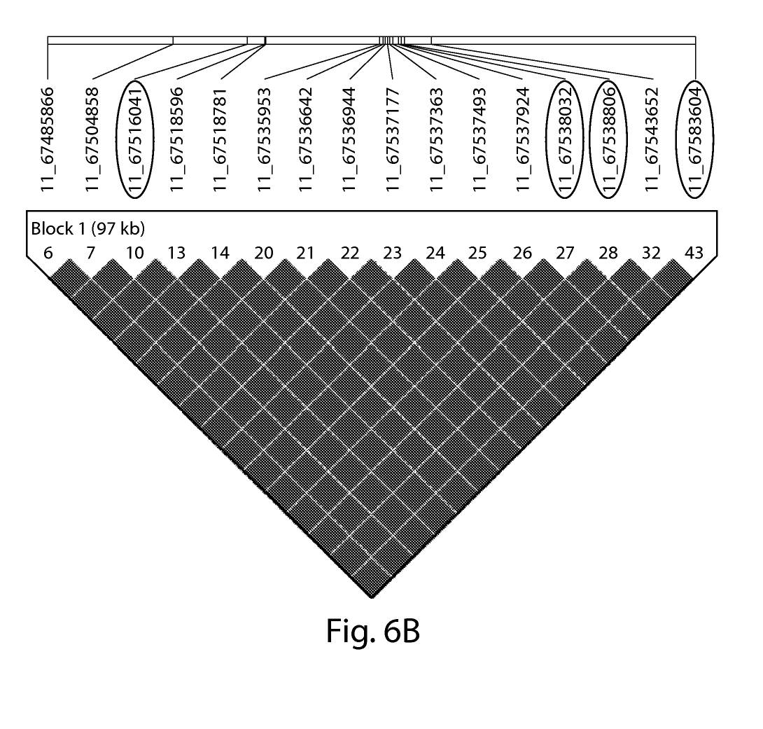

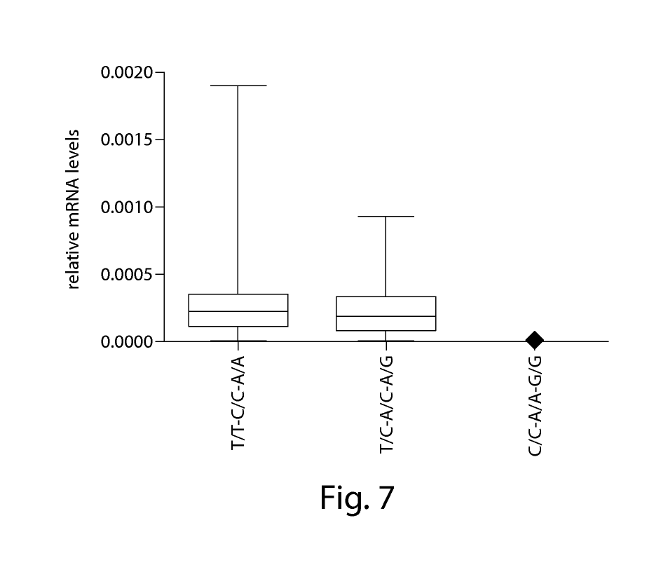

To examine the functional effect of the risk haplotypes the mRNA expression of PTPN3 and all eight genes on chromosome 32 locus were measured in peripheral blood mononuclear cells (PBMC) from 165 healthy NSDTRs (FIG. 4). The 15 SNP risk haplotype on chromosome 11 overlaps the PTPN3 gene and the LD is almost complete between these SNPs (r²>0.95 for all pairs) (FIGS. 5 and 6). Therefore three of these SNPs (one in the 3′UTR, a SNP in intron 18 and one synonymous SNP in exon 18, CanFam2.0 positions, 11:67,516,041, 11:67,538,032 and 11:67,538,806, respectively) were selected and the genotypes were correlated with mRNA expression. While the expression of PTPN3 was substantially down-regulated in heterozygotes and only one homozygous dog available compared to homozygotes for the protective alleles, the difference was not significant (FIG. 7). We also genotyped one adjoining SNP falling just outside the haplotype (R²=0.25, D′=1), but also associated to the disease (p=7.5×10⁻⁴) (Table 11) and located in exon 3 (11:67,583,604, FIGS. 5 and 6). Interestingly, when a four SNP haplotype was generated (FIG. 6), the association to expression was even stronger (8-fold change, p=0.0129, FIG. 1). Among the cases used in the association study, only four dogs were homozygous for the risk haplotype C/C-A/A-G/G-T/T (11:67,516,041, 11:67,538,032, 11:67,538,806 and 11:67,583,604, respectively) and all of them showed severe IMRD that eventually led to death. In the healthy controls used in the expression study, no dogs homozygous for the risk haplotype were present, and hence no expression data was available to evaluate the homozygous risk state for this four SNP haplotype. However, by extrapolation it is speculated that the homozygous state would lead to severe down-regulation of PTPN3 mRNA expression. The increased risk seen when combining the three SNPs from the large risk haplotype with the separate exon 3 SNP suggests that there may be two mutations acting together.

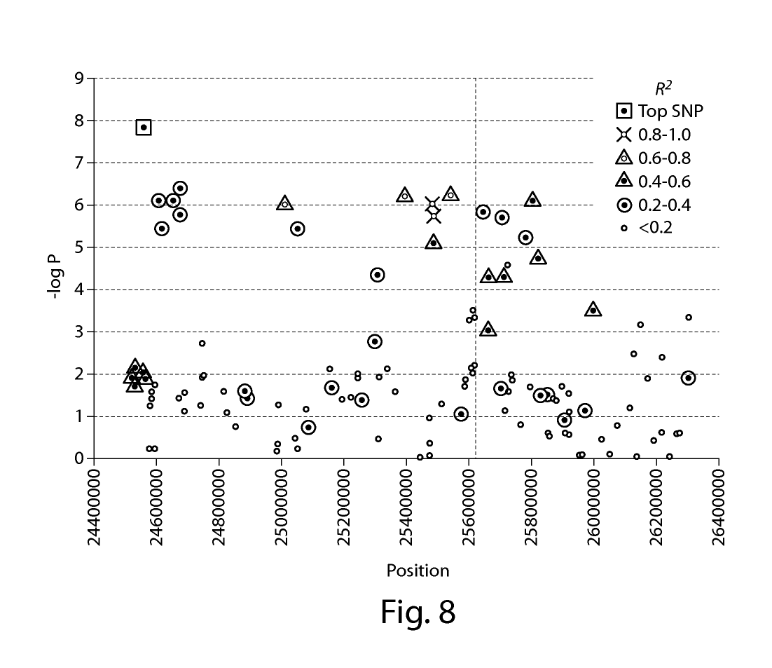

Within the 1.3 Mb region on chromosome 32 spanning eight genes (chr32:24,556,037-25,816,401), the association curve fluctuates and 23 SNPs show association of p<10⁻⁵, making it difficult to pinpoint a specific gene relevant to the pathogenesis of IMRD (Table 12). The LD in the region was examined and compared to the most highly associated SNP (32: 24,556,037, FIG. 1b, FIG. 8) and it was noted that only two SNPs, (32:25,485,961 and 32:25,485,644) were in strong LD (r2>0.8) with this SNP. 12 highly associated SNPs spanning the region were genotyped and expression of all the genes located in the region was correlated with each of these SNPs. Only two genes, DDIT4L and BANK1, show significant expression differences related to the most highly associated SNP, although multiple genes showed non-significant changes (Table 13). The mRNA expression of both genes was up-regulated in PBMCs from individuals carrying the risk allele at the top SNP (32:24,556,037, DDIT4L: 3-fold, p=0.004; BANK1: 1.3-fold, p=0.003). The resulting two SNP haplotype also gave significant expression changes for both genes (p=0.0002 and p=0.006, respectively, FIG. 1). Among the other twelve SNPs only the SNP at position 32:25,485,961 gave a significant association to any gene, namely to DDIT4L (3.5 fold-change, p=0.0001) and BANK1 (1.3 fold-change, p=0.005) (Table 13).

TABLE 13
Association of differential expression of DDIT4L, BANK1 and
DAPP1 with variants across the chromosome 32 locus.

| | DDIT4 | Means fold- | BANK1 | Means fold- | Best other gene |
|---|---|---|---|---|---|
| SNP | P-value | change | P-value | change | P-value/fold-change |
| 32:24556037 | 0.004 | 3X | 0.003 | 1.3X | PPP3CA, 0.12, 1.1X |
| 32:24606503¹ | 0.01 | 1.9X | 0.50 | 1.1X | LAMTOR1, 0.25, 1.2X |
| 32:24667283¹ | 0.01 | 1.9X | 0.50 | 1.1X | LAMTOR1, 0.23, 1.2X |
| 32:24672221¹ | 0.01 | 1.9X | 0.47 | 1.15X | LAMTOR1, 0,23, 1.2X |
| 32:25007496 | 0.0002 | 3.1X | 0.006 | 1.2X | DNAJB14, 0.09, 1.1X |
| 32:25305524¹ | 0.03 | 1.8X | 0.55 | 1.1X | LAMTOR1, 0.35, 1.1X |
| 32:25485961 | 0.0001 | 3.5X | 0.005 | 1.3X | DNAJB14, 0.20, ND |
| 32:25537276 | 0.03 | 2.2X | 0.02 | 1.3X | DNAJB14, 0.12, 1.3X |
| 32:25642357¹ | 0.02 | 1.8X | 0.48 | 1.1X | LAMTOR1, 0.20, 1.2X |
| 32:25702963¹ | 0.02 | 1.8X | 0.48 | 1.1X | LAMTOR1, 0.20, 1.2X |
| 32:25779083¹ | 0.002 | 2X | 0.80 | ND | PPP3CA, 0.15, 1.2X |
| 32:25798353¹ | 0.30 | 1.6X | 0.05 | 1.2X | PPP3CA, 0.25,1.1X |

Correlation was performed by ANOVA,
ND—no difference
¹—correlation for SNPs with missing homozygotes for the minor allele was performed for homozygotes versus heterozygotes with unpaired t-test.

The proteins encoded by the PTPN3, DDIT4L, and BANK1 genes have multiple functions in signal transduction pathways depending on the cell context. Human protein tyrosine phosphatase PTPH1 encoded by the PTPN3 gene inhibits T cell-activation by dephosphorylating the immune tyrosine-based activation motifs (ITAM) in the TCRζ chain that results in a down-stream inhibition of NF-AT [refs. 18,19]. The observed substantial reduction of the PTPN3 mRNA levels in dogs carrying the risk haplotypes may cause a sustained activation of TCR signaling and lead to development of autoimmune disease.

The precise role of the protein encoded by the DDIT4L gene remains largely unknown. Its function in autoimmunity may be related to the negative regulation of mTOR [ref. 20]. Interestingly, inhibition of mTOR promotes generation of CD8+ memory T cells [ref. 21]. DDIT4L mRNA expression is also up-regulated in macrophages in response to LPS [ref. 22]. Moreover, overexpression of DDIT4L cDNA in U-937 monocytes induced cell death by necrosis [ref. 23] which may trigger an auto-inflammatory response.

The gene encoding the B-cell scaffold protein with ankyrin repeats, BANK1, is of special interest. It was previously found associated with human SLE and other autoimmune diseases in distinct populations and ethnic groups [refs. 24-26]. The expression of the human BANK1 gene has been reported to be up-regulated in patients carrying human risk alleles [ref. 24,27]. In both cases, a moderate up-regulation of ˜30% appears to contribute to disease risk.

In summary, the study herein displays strong evidence for an association between the ANA^{S} group and MHC class II genotype 2 (OR=33.2 and p<0.0001) together with the risk loci on chromosomes 11 and 32. These loci appear to contribute to the risk for the speckled ANA phenotype through altered mRNA expression levels of the PTPN3, DDIT4L and BANK1 genes. At both loci multiple genes and/or mutations appear to play a role suggesting that while the IMRD phenotype in the NSDTR breed is associated with a few loci of strong effect, these loci may play an important and complex function for the disease primarily through altered gene regulation. In human GWAS studies, identification of the actual mutations may be complicated due to the presence of multiple candidate variants within non-coding regions of the genome.

### Methods

### Study Population and Diagnostic Procedures

142 Nova Scotia duck tolling retrievers (NSDTRs) were included in this study, 64 of them classified as ANA-positive IMRD cases and 78 as healthy controls. All dogs included were privately owned and samples were collected during 2002-2012. Individual dog owners had consulted different veterinary clinics in Sweden and Finland. The inclusion criteria for IMRD ANA-positive dogs were musculoskeletal signs indicating a systemic rheumatic disorder, including stiffness mainly after rest, and pain from several joints of extremities. These signs had to be apparent for at least 14 days and were the main reason for the dog owner to visit the veterinary clinic. The examining veterinary physician suspected no other diseases in their diagnosis. All dogs should also display a positive IIF ANA test. Healthy controls were above seven years of age with no history of autoimmune disease.

ANA tests were analyzed with indirect immunofluorescence at the University Animal Hospital, Swedish University of Agricultural Sciences (SLU), Uppsala, Sweden using monolayers of HEp-2 cells fixed on glass slides (Immuno Concepts). The glass slides were examined by fluorescence microscopy and considered positive at a titer of ≧1:100. The visible nuclear fluorescence patterns could be divided into two groups; homogeneous (ANA^{H}) or speckled (ANA^{S}) patterns as previously described [ref. 8].

### DNA Purification, PCR Amplification of DLA Regions and Sequence Analysis

Genomic DNA was purified from 200 μl of blood using Qiagen QIAamp DNA Blood Mini Kit (Qiagen) according to the manufacturer's protocol. DLA-DRB1, -DQA1 and DQB1 exon 2 were amplified by PCR as previously described [ref. 16]. DNA sequencing was performed using capillary electrophoresis on an Applied Biosystems 3730x1. BigDye® Terminator v3.1 (Applied Biosystems) Sequencing of the purified PCR products was made in one direction, reverse for DLA-DRB1 and -DQA1 and forward for DLA-DQB1. Analysis of the nucleotide sequence was performed using MatchTools and MatchTools Navigator (Applied Biosystems) [ref. 16].

### Statistical Analysis

Statistical analyses were performed using VassarStats (vassarstats.net/odds2×2.html). Odds ratios and p-values for each allele, haplotype and genotype were calculated using a 2×2 contingency table. The total number of cases and controls carrying a specific allele or genotype was compared with the cases and controls not carrying it. The same comparison was made for alleles as well as genotypes for the ANA-positive cases with homogeneous or speckled pattern and the controls. The total numbers of homozygous dogs were also compared in cases and controls.

### Next Generation Re-Sequencing

To identify candidate variants, the five regions previously found associated with IMRD [ref. 2], spanning approximately 5 Mb, were re-sequenced in seven individuals (four ANA cases and three controls) using 385K custom designed capture arrays from NimbleGen and 400-600 X coverage Illumina sequencing. The sequencing data was aligned with BWA [ref. 28] and analyzed using SAMtools [ref. 29], BEDTools [ref. 30], SEQscoring [ref. 31] and other in-house tools to discover variants (SNPs and indels) in the genomic sequence between IMRD and healthy control dogs.

### SNP Selection for Genotyping and Sssociation Analysis

Of the 26 ANA^{S}-positive NSDTRs with MHC class II genotype 2, 25 were included for additional analysis as well as 145 healthy controls and a total of 63 ANA-positive dogs (regardless of ANA staining pattern). 384 SNPs for five loci (chromosome 3, 8, 11, 24 and 32) were chosen from the re-sequencing data. For genotyping we selected variants located in the conserved non-coding and protein coding regions which was assessed by using SiPhy [ref. 32]. Theses SNPs were genotyped by GoldenGate® Genotyping Assay. PLINK [ref. 33] was used to analyze the markers with a MAF>0.05 and a call rate>0.75. Total genotyping rate was 97.3%. All SNPs that reached a Bonferroni corrected p-value were considered highly significant.

RNA Extraction and cDNA Synthesis

Peripheral blood was drawn from 165 healthy NSDTR dogs directly in Tempus Blood RNA tubes (Applied Biosystems) and kept on ice during transportation. Total blood RNA was purified using the Tempus Spin RNA Isolation Reagent kit (Applied Biosystems) according to the manufacturer's instructions. In parallel, genomic DNA was purified for each sample and genotyped using pyrosequencing or direct Sanger sequencing with the primers shown in Table 14. cDNA synthesis was performed at 42 degrees C. for 80 min using 2 μg of RNA, 5 μM oligo-dT primer, MuLV transcriptase, RNase inhibitor in the buffer supplemented with 5 mM MgCl2 and 1 mM dNTPs. All reagents were from Applied Biosystems. The reaction was terminated by heating for 5 min at 95 degrees C. and diluted to 25 ng/μl.

TABLE 14
Primer pairs used for genotyping in expression samples.

| | | | | | Annealing | |
|---|---|---|---|---|---|---|
| CFA | Position | Alleles | Primers | Primer sequences | Temp, ° C. | Method |
| 11 | 67538032 | G/T | F | GATGGATTGCTAGCAGAATGAA (SEQ ID NO: 15) | 50 | Pyrosequence |
| | | | R | CACGACGTTGTAAAACGACGTAGTCGCTAATCA | | |
| | | | | CGATCTAT (SEQ ID NO: 16) | | |
| 11 | 67516041 | A/G | F | CACGACGTTGTAAAACGACCTGCCCTGCGTTCC | 50 | Pyrosequence |
| | | | | TCTATCCC (SEQ ID NO: 17) | | |
| | | | R | CTGAAAACTCGCCCGAACCT (SEQ ID NO: 18) | | |
| 11 | 67538806 | T/C | F | TGACATCAAATCCGACGATGAG (SEQ ID NO: 19) | 50 | Pyrosequence |
| | | | R | CACGACGTTGTAAAACGACGATCAGCACCGTCC | | |
| | | | | CGCTTTC (SEQ ID NO: 20) | | |
| 32 | 24987404 | G/A | F | CAGTCGCGGTCGCTTCTCATCT (SEQ ID NO: 21) | 57 | Pyrosequence |
| | | | R | CACGACGTTGTAAAACGACGCAGCTGCAGAGC | | |
| | | | | TTTATGAC (SEQ ID NO: 22) | | |
| 32 | 24985562 | G/A | F | CTATTTTTGACAATAAAGCATC (SEQ ID NO: 23) | 50 | Pyrosequence |
| | | | R | CACGACGTTGTAAAACGACTACAATTAAGGAA | | |
| | | | | ACGAATTGC (SEQ ID NO: 24) | | |
| 32 | 24606503 | T/C | F | TTCCTCAGGTTGAGGGTT (SEQ ID NO: 25) | 52 | Pyrosequence |
| | | | R | CACGACGTTGTAAAACGACGCTTCAATGTACTC | | |
| | | | | TTGTAGTT (SEQ ID NO: 26) | | |
| 32 | 25798353 | G/A | F | CACGACGTTGTAAAACGACATTAAGAATAGATC | 55 | Pyrosequence |
| | | | | CTCCTACA (SEQ ID NO: 27) | | |
| | | | R | ACTATCTACTGGCAGGTATCCA (SEQ ID NO: 28) | | |
| 32 | 25714903 | T/C | F | CACGACGTTGTAAAACGACTGAGGTCGAAGGA | 50 | Pyrosequence |
| | | | | GGAGAGATG (SEQ ID NO: 29) | | |
| | | | R | ATCCCTAGCATACTAGACTTTC (SEQ ID NO: 30) | | |
| 32 | 25512953 | T/A | F | GAAAGATTCTAAATCCTTGAAC (SEQ ID NO: 31) | 50 | Pyrosequence |
| | | | R | CACGACGTTGTAAAACGACTCTAATAGCATCAT | | |
| | | | | TTATCA (SEQ ID NO: 32) | | |
| 32 | 26115349 | A/T | F | GTCAGCCTCCTGGGTATTTGTA (SEQ ID NO: 33) | 55 | Pyrosequence |
| | | | R | CACGACGTTGTAAAACGACTGGAACTGCTGTTT | | |
| | | | | TAATGT (SEQ ID NO: 81) | | |
| 32 | 25079168 | A/G | F | CACGACGTTGTAAAACGACGCTTTAGAGCAACC | 55 | Pyrosequence |
| | | | | ACCTAA (SEQ ID NO: 34) | | |
| | | | R | TCCTTGTGTATCCCATGCCAA (SEQ ID NO: 35) | | |
| 32 | 25363099 | G/A | F | CACGACGTTGTAAAACGACTGCAAAATTCAACT | 55 | Pyrosequence |
| | | | | GTAATG (SEQ ID NO: 36) | | |
| | | | R | CCATACATCACCGACCCTCAGC (SEQ ID NO: 37) | | |
| 32 | 24890208 | A/G | F | CACGACGTTGTAAAACGACAGGTAATGGAGTA | 52 | Pyrosequence |
| | | | | ATGTAAGT (SEQ ID NO: 38) | | |
| | | | R | GGAAAATTTAGTGGCCTGTGTT (SEQ ID NO: 39) | | |
| 32 | 24827518 | C/T | F | CACGACGTTGTAAAACGACGGTTCAAATCCCAA | 50 | Pyrosequence |
| | | | | GATCAAGT (SEQ ID NO: 40) | | |
| | | | R | GGTTCAAATCCCAAGATCAAGT (SEQ ID NO: 82) | | |
| 32 | 24556037 | G/A | F | AATTCAGCAAGTTACCTTATCA (SEQ ID NO: 41) | 50 | Sanger |
| | | | R | CTTAGCATTATACTCTCTTGGT (SEQ ID NO: 42) | | sequence |
| 32 | 24667283 | C/T | F | GATAAGGGTTGAAAGAATAGGCAAG | 50 | Sanger |
| | | | | (SEQ ID NO: 43) | | sequence |
| | | | R | CAAACAGCCTAGAGTCACTTTCT | | |
| | | | | (SEQ ID NO: 44) | | |
| 32 | 24672221 | G/A | F | TGAGTCTCGAGGATTGAATGACT | 50 | Sanger |
| | | | | (SEQ ID NO: 45) | | sequence |
| | | | R | CCTAGGGTGATTTTGTGTAAGCT | | |
| | | | | (SEQ ID NO: 46) | | |
| 32 | 25007496 | A/G | F | TAAAAGCATGAGGGAAACAGCATC | 50 | Sanger |
| | | | | (SEQ ID NO: 47) | | sequence |
| | | | R | TAATTCTTTCAGTGAGGGCATTATAG | | |
| | | | | (SEQ ID NO: 48) | | |
| 32 | 25305524 | A/G | F | CCCCGAGCTACAGAGATGGA (SEQ ID NO: 49) | 55 | Sanger |
| | | | R | AGCACAGCCCCTGTGAAAAT (SEQ ID NO: 50) | | sequence |
| 32 | 25485961 | A/G | F | CATGGCAACCCAAAGGCAAC (SEQ ID NO: 51) | 55 | Sanger |
| | | | R | CCCCTTCACAGATACCCTGC (SEQ ID NO: 52) | | sequence |
| 32 | 25537276 | C/T | F | TGGTCTGAGCCTGAAAGTGG (SEQ ID NO: 53) | 55 | Sanger |
| | | | R | TGCTGCTGCTGTTAAAGGGT (SEQ ID NO: 54) | | sequence |
| 32 | 25642357 | A/T | F | TCTCTCCTCTTTAGCTTCTGCC (SEQ ID NO: 55) | 55 | Sanger |
| | | | R | TCCCTGGTTGGAAATGAGCC (SEQ ID NO: 56) | | sequence |
| 32 | 25702963 | A/G | F | TGCGCTTTAAGACACGTGGA (SEQ ID NO: 57) | 55 | Sanger |
| | | | R | GCAAAGTGCAAGCAAGGTGA (SEQ ID NO: 58) | | sequence |
| 32 | 25779083 | A/C | F | TCCGTCAAATTGTTTCTCATGTTGA | 50 | Sanger |
| | | | | (SEQ ID NO: 59) | | sequence |
| | | | R | TGAGTACCTTAACAGTTCAGAGC | | |
| | | | | (SEQ ID NO: 60) | | |

### Quantitative RT-PCR

Gene expression was measured by quantitative real-time PCR on 7900HT Sequence Detector (Applied Biosystems) with SDS 2.3 software using SYBR Green for signal detection. Gene-specific primers and annealing Tm are shown in Table 15. Initial denaturation at 95 degrees C. for 5 min was followed by 45 cycles (95 degrees C. for 15 s, annealing at primer-specific Tm for 15 s and 72 degrees C. for 25 s). PCR buffer was supplemented with 1.5 mM MgCl2, 200 μM of each dNTPs, primers, SYBRGreen (Molecular Probes), 15 ng of cDNA and 0.5 U of Platinum Taq polymerase (Invitrogen). Expression levels were normalized to TBP using the comparative 2^{-ΔCt}-method [ref. 34]. All experiments were run in triplicate. Correlation of gene expression with genotypes and haplotypes was performed using one-way ANOVA tests in PRISM 6 (GraphPad Software).

TABLE 15
Primers for qRT-PCR.

| | | | |
|---|---|---|---|
| | | Annealing | Amplicon |
| Primers | Sequence | T, 0° C. | length |

PTPN3

| | | | |
|---|---|---|---|
| PTPN3-ex2-3-for | AGTCGACTCTCTGAGATGGCTG | 60 | 142 bp |
| | (SEQ ID NO: 61) | | |
| Rev-PTPN3-ex4-5 | CAAATGCCTGGTTTGTTCTTGCT | | |
| | (SEQ ID NO: 62) | | |

DAPP1

| | | | |
|---|---|---|---|
| DAPP1-Ex1-2-f | AGGAATTGGGGTGGTATCACG | 60 | 165 bp |
| | (SEQ ID NO: 63) | | |
| Rev-ex3-DAPP1 | CAACATGAAAGTGTTTGACAGAGTC | | |
| | (SEQ ID NO: 64) | | |

LAMTOR3

| | | | |
|---|---|---|---|
| LAMTOR-ex5-6-for | AACACCTACCAGGTGGTTCAATTCA | 60 | 157 bp |
| | (SEQ ID NO: 65) | | |
| Rev-ex7-LAMTOR3 | TGCCAGATTAAGAAACTTCCACCACT | | |
| | (SEQ ID NO: 66) | | |

DNAJB14

| | | | |
|---|---|---|---|
| DNAJB14-ex6-7- | AGATCTGGATCAGGGCAAAC | 60 | 207 bp |
| for | (SEQ ID NO: 67) | | |
| Rev-NEW-ex8- | TACTTTGGCTGCATACTGCATATC | | |
| DNAJB14 | (SEQ ID NO: 68) | | |

H2AFZ

| | | | |
|---|---|---|---|
| H2AFZ-ex3-4-for | ACCGCAGAGGTACTTGAATTG | 60 | 151 bp |
| | (SEQ ID NO: 69) | | |
| Rev-ex4-5-H2AFZ | GTGGAATAACACCACCACCAG | | |
| | (SEQ ID NO: 70) | | |

DDIT4L

| | | | |
|---|---|---|---|
| DDIT4L-ex1-for | CATGGTGGCAACTGGCAGTTTGA | 60 | 105 bp |
| | (SEQ ID NO: 71) | | |
| Rev-ex1-2-DDIT4L | CAGTAGTCAAAATCATTTAGCAGGC | | |
| | (SEQ ID NO: 72) | | |

EMCN

| | | | |
|---|---|---|---|
| EMCN-For-qPCR | CAGACCCAGGCACACCAGAA | 60 | 110 bp |
| | (SEQ ID NO: 73) | | |
| Rev-EMCN-qPCR | TGCAGAGTGCTCACCAGACTCAT | | |
| | (SEQ ID NO: 74) | | |

PPP3CA

| | | | |
|---|---|---|---|
| PPP3CA-ex2-for | TAATAACAGAAGGGGCTTCAATTC | 60 | 175 bp |
| | (SEQ ID NO: 75) | | |
| Rev-ex3-PPP3CA | CTGTCAACATAGTCCCCTAAGAAG | | |
| | (SEQ ID NO: 76) | | |

BANK1

| | | | |
|---|---|---|---|
| K9-BANK1-for2 | GTATTCAGAGGTTCTGAGGACTA | 63 | 176 bp |
| | (SEQ ID NO: 77) | | |
| BANK1-rev-K9 | TCACCAGGATTCTCACATGGAAT | | |
| | (SEQ ID NO: 78) | | |

TBP house-keeping gene

| | | | |
|---|---|---|---|
| TBP-ex5-forw | TCAGTTCTGGGAAGATGGTGTGTA | 63 | 218 bp |
| | (SEQ ID NO: 79) | | |
| Rev-ex6-7-TBP | CTCTGGCTCGTAACTGCTAAACT | | |
| | (SEQ ID NO: 80) | | |

### REFERENCES

1. Tan, E. M. Antinuclear antibodies: diagnostic markers for autoimmune diseases and probes for cell biology. Adv Immunol 44, 93-151 (1989).
2. Wilbe, M. et al. Genome-wide association mapping identifies multiple loci for a canine SLE-related disease complex. Nat Genet 42, 250-4 (2010).
3. Hansson-Hamlin, H. & Lilliehook, I. A possible systemic rheumatic disorder in the Nova Scotia duck tolling retriever. Acta Vet Scand 51, 16 (2009).
4. Rahman, A. & Isenberg, D. A. Systemic lupus erythematosus. N Engl J Med 358, 929-39 (2008).
5. D'Cruz, D. Testing for autoimmunity in humans. Toxicol Lett 127, 93-100 (2002).
6. Gershwin, L. J. Autoimmune diseases in small animals. Vet Clin North Am Small Anim Pract 40, 439-57 (2010).
7. Hay, E. M. Systemic lupus erythematosus. Baillieres Clin Rheumatol 9, 437-70 (1995).
8. Hansson-Hamlin, H., Lilliehook, I. & Trowald-Wigh, G. Subgroups of canine antinuclear antibodies in relation to laboratory and clinical findings in immune-mediated disease. Vet Clin Pathol 35, 397-404 (2006).
9. Arbuckle, M. R. et al. Development of autoantibodies before the clinical onset of systemic lupus erythematosus. N Engl J Med 349, 1526-33 (2003).
10. Lyons, R., Narain, S., Nichols, C., Satoh, M. & Reeves, W. H. Effective use of autoantibody tests in the diagnosis of systemic autoimmune disease. Ann N Y Acad Sci 1050, 217-28 (2005).
11. Wilbe, M. et al. DLA class II alleles are associated with risk for canine symmetrical lupoid onychodystrophy [corrected](SLO). PLoS One 5, e12332 (2010).
12. Kennedy, L. J. et al. Identification of susceptibility and protective major histocompatibility complex haplotypes in canine diabetes mellitus. Tissue Antigens 68, 467-76 (2006).
13. Kennedy, L. J. et al. Association of canine hypothyroidism with a common major histocompatibility complex DLA class II allele. Tissue Antigens 68, 82-6 (2006).
14. Ollier, W. E. et al. Dog MHC alleles containing the human RA shared epitope confer susceptibility to canine rheumatoid arthritis. Immunogenetics 53, 669-73 (2001).
15. Wilbe, M. et al. Increased genetic risk or protection for canine autoimmune lymphocytic thyroiditis in Giant Schnauzers depends on DLA class II genotype. Tissue Antigens 75, 712-9 (2010).
16. Wilbe, M. et al. MHC class II polymorphism is associated with a canine SLE-related disease complex. Immunogenetics 61, 557-64 (2009).
17. Guerra, S. G., Vyse, T. J. & Cunninghame Graham, D. S. The genetics of lupus: a functional perspective. Arthritis Res Ther 14, 211 (2012).
18. Han, S., Williams, S. & Mustelin, T. Cytoskeletal protein tyrosine phosphatase PTPH1 reduces T cell antigen receptor signaling. Eur J Immunol 30, 1318-25 (2000).
19. Sozio, M. S. et al. PTPH1 is a predominant protein-tyrosine phosphatase capable of interacting with and dephosphorylating the T cell receptor zeta subunit. J Biol Chem 279, 7760-9 (2004).
20. Corradetti, M. N., Inoki, K. & Guan, K. L. The stress-inducted proteins RTP801 and RTP801L are negative regulators of the mammalian target of rapamycin pathway. J Biol Chem 280, 9769-72 (2005).
21. Rao, R. R., Li, Q., Odunsi, K. & Shrikant, P. A. The mTOR kinase determines effector versus memory CD8+ T cell fate by regulating the expression of transcription factors T-bet and Eomesodermin. Immunity 32, 67-78 (2010).
22. Iliev, D. B., Goetz, G. W., Mackenzie, S., Planas, J. V. & Goetz, F. W. Pathogen-associated gene expression profiles in rainbow trout macrophages. Comp Biochem Physiol Part D Genomics Proteomics 1, 416-22 (2006).
23. Cuaz-Perolin, C. et al. REDD2 gene is upregulated by modified LDL or hypoxia and mediates human macrophage cell death. Arterioscler Thromb Vasc Biol 24, 1830-5 (2004).
24. Kozyrev, S. V. et al. Functional variants in the B-cell gene BANK1 are associated with systemic lupus erythematosus. Nat Genet 40, 211-6 (2008).
25. Chang, Y. K. et al. Association of BANK1 and TNFSF4 with systemic lupus erythematosus in Hong Kong Chinese. Genes Immun 10, 414-20 (2009).
26. Yang, W. et al. Genome-wide association study in Asian populations identifies variants in ETS1 and WDFY4 associated with systemic lupus erythematosus. PLoS Genet 6, e1000841 (2010).
27. Kozyrev, S. V., Bernal-Quiros, M., Alarcon-Riquelme, M. E. & Castillejo-Lopez, C. The dual effect of the lupus-associated polymorphism rs10516487 on BANK1 gene expression and protein localization. Genes Immun 13, 129-38 (2012).
28. Li, H. & Durbin, R. Fast and accurate long-read alignment with Burrows-Wheeler transform. Bioinformatics 26, 589-95 (2010).
29. Li, H. et al. The Sequence Alignment/Map format and SAMtools. Bioinformatics 25, 2078-9 (2009).
30. Quinlan, A. R. & Hall, I. M. BEDTools: a flexible suite of utilities for comparing genomic features. Bioinformatics 26, 841-2 (2010).
31. Truve, K. et al. SEQscoring: a tool to facilitate the interpretation of data generated with next generation sequencing technologies. EMBnet journal 17, 38-45 (2011).
32. Garber, M. et al. Identifying novel constrained elements by exploiting biased substitution patterns. Bioinformatics 25, i54-62 (2009).
33. Purcell, S. et al. PLINK: a tool set for whole-genome association and population-based linkage analyses. Am J Hum Genet 81, 559-75 (2007).
34. Livak, K. J. & Schmittgen, T. D. Analysis of relative gene expression data using real-time quantitative PCR and the 2(-Delta Delta C(T)) Method. Methods 25, 402-8 (2001).

Without further elaboration, it is believed that one skilled in the art can, based on the above description, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

The indefinite articles “a” and “an,” as used herein in the specification and in the claims, unless clearly indicated to the contrary, should be understood to mean “at least one.”

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method, comprising:
a) analyzing genomic DNA from a canine subject for the presence of a single nucleotide polymorphism (SNP) selected from chr11:67543652, chr11:67538032, chr11:67516041, chr11:67537363, chr11:67538806, chr11:67537493, chr11:67536944, and chr11:67583604; and
b) identifying a canine subject having the SNP as a subject at elevated risk of developing IMRD or having undiagnosed IMRD.

2. The method of claim 1, wherein the canine subject is homozygous for the DLA haplotype DLA-BRB1*00601, DQA1*005011, and DQB1*02001.

3. A method, comprising:
a) analyzing genomic DNA from a canine subject for the presence of a single nucleotide polymorphism (SNP) selected from chr11:67543652, chr11:67538032, chr11:67516041, chr11:67537363, chr11:67538806, chr11:67537493, chr11:67536944, and chr11:67583604;
b) analyzing the genomic DNA for the presence of a DLA haplotype DLA-BRB1*00601, DQA1*005011, and DQB1*02001; and
c) identifying a canine subject having the SNP and homozygous for the DLA haplotype as a subject at elevated risk of developing IMRD or having undiagnosed IMRD.

4. The method of any one of claims 1 to 3, wherein the SNP is a SNP at chromosome position chr11:67583604.

5. The method of any one of claims 1 to 4, wherein the genomic DNA is obtained from a bodily fluid or tissue sample of the subject.

6. The method of any one of claims 1 to 5, wherein the genomic DNA is analyzed using a single nucleotide polymorphism (SNP) array.

7. The method of any one of claims 1 to 5, wherein the genomic DNA is analyzed using a bead array.

8. The method of any one of claims 1 to 5, wherein the genomic DNA is analyzed using a nucleic acid sequencing assay.

9. The method of claim 1 or 3, wherein the SNP is two or more SNPs.

10. The method of claim 1 or 3, wherein the SNP is three or more SNPs.

11. A method, comprising:
(a) analyzing genomic DNA from a canine subject for the presence of a risk haplotype selected from a risk haplotype having chromosome coordinates chr11:67536642-67583604; and
(b) identifying a canine subject having the risk haplotype as a subject at elevated risk of developing IMRD or having undiagnosed IMRD.

12. The method claim 11, wherein the canine subject is homozygous for the DLA haplotype DLA-BRB1*00601, DQA1*005011, and DQB1*02001.

13. A method, comprising:
a) analyzing genomic DNA from a canine subject for the presence of a risk haplotype selected from a risk haplotype having chromosome coordinates chr11:67536642-67583604;
b) analyzing the genomic DNA for the presence of a DLA haplotype DLA-BRB1*00601, DQA1*005011, and DQB1*02001; and
c) identifying a canine subject having the risk haplotype and homozygous for the DLA haplotype as a subject at elevated risk of developing IMRD or having undiagnosed IMRD.

14. The method of any one of claims 11 to 13, wherein the presence of the risk haplotype is detected by analyzing the genomic DNA for the presence of a SNP located within the risk haplotype.

15. The method of claim 14, wherein the SNP is selected from a SNP at chromosome position chr11:67543652, chr11:67538032, chr11:67516041, chr11:67537363, chr11:67538806, chr11:67537493, chr11:67536944, and chr11:67583604.

16. The method of any one of claims 11 to 15, wherein the genomic DNA is obtained from a bodily fluid or tissue sample of the subject.

17. The method of any one of claims 11 to 16, wherein the genomic DNA is analyzed using a single nucleotide polymorphism (SNP) array.

18. The method of any one of claims 11 to 16, wherein the genomic DNA is analyzed using a bead array.

19. The method of any one of claims 11 to 16, wherein the genomic DNA is analyzed using a nucleic acid sequencing assay.

20. The method of any one of claims 11 to 16, wherein the risk haplotype is two risk haplotypes.

21. The method of claim 14, wherein the SNP is two or more SNPs.

22. The method of claim 14, wherein the SNP is three or more SNPs.

23. A method, comprising:
(a) analyzing genomic DNA from a canine subject for the presence of a mutation in a gene selected from PTPN3 and BANK1; and
(b) identifying a canine subject having the mutation as a subject at elevated risk of developing IMRD or having undiagnosed IMRD.

24. The method claim 23, wherein the canine subject is homozygous for the DLA haplotype DLA-BRB1*00601, DQA1*005011, and DQB1*02001.

25. A method, comprising:
(a) analyzing genomic DNA from a canine subject for the presence of a mutation in a gene selected from PTPN3 and BANK1;
b) analyzing the genomic DNA for the presence of a DLA haplotype DLA-BRB1*00601, DQA1*005011, and DQB1*02001; and
(c) identifying a canine subject having the mutation and homozygous for the DLA haplotype as a subject at elevated risk of developing IMRD or having undiagnosed IMRD.

26. The method of any one of claims 23 to 25, wherein the gene is PTPN3.

27. The method of any one of claims 23 to 25, wherein the gene is BANK1.

28. The method of any one of claims 23 to 27, wherein the genomic DNA is obtained from a bodily fluid or tissue sample of the subject.

29. The method of any one of claims 23 to 28, wherein the genomic DNA is analyzed using a single nucleotide polymorphism (SNP) array.

30. The method of any one of claims 23 to 28, wherein the genomic DNA is analyzed using a bead array.

31. The method of any one of claims 23 to 28, wherein the genomic DNA is analyzed using a nucleic acid sequencing assay.

32. The method of any one of claims 23 to 25, wherein the mutation is two or more mutations.

33. The method of any one of claims 23 to 25, wherein the mutation is three or more mutations.

34. The method of any one of claims 23 to 25, wherein the gene is two or more genes.

35. The method of any one of claims 23 to 25, wherein the gene is three or more genes.

36. A method, comprising:
(a) analyzing a sample from a canine subject for a level of PTPN3 and/or BANK1; and
(b) identifying a canine subject having a decreased level of PTPN3 and/or an elevated level of BANK1 compared to a control level as a subject at elevated risk of developing IMRD or having undiagnosed IMRD.

37. The method of any one of claims 1 to 36, wherein the IMRD is ANA-positive IMRD.

38. The method of any one of claims 1 to 37, wherein the IMRD is speckled ANA-positive IMRD.

39. The method of any one of claims 1 to 38, wherein the canine subject is a descendent of a Nova Scotia duck tolling retriever.

40. The method of any one of claims 1 to 39, wherein the canine subject is a Nova Scotia duck tolling retriever.

41. A method, comprising:
(a) analyzing genomic DNA in a sample from a subject for presence of a mutation in a gene selected from PTPN3, or an orthologue of such a gene, and, BANK1, or an orthologue of such a gene; and
(b) identifying a subject having the mutation as a subject at elevated risk of developing SLE or an SLE-related disease or having undiagnosed SLE or an SLE-related disease.

42. The method of claim 41, wherein the subject is a human subject.

43. The method of claim 41, wherein the subject is a canine subject.

44. The method of any one of claims 41 to 43, wherein the gene is PTPN3.

45. The method of any one of claims 41 to 43, wherein the gene is BANK1.

46. The method of any one of claims 41 to 45, wherein the genomic DNA is obtained from a bodily fluid or tissue sample of the subject.

47. The method of any one of claims 41 to 46, wherein the genomic DNA is analyzed using a single nucleotide polymorphism (SNP) array.

48. The method of any one of claims 41 to 46, wherein the genomic DNA is analyzed using a bead array.

49. The method of any one of claims 41 to 46, wherein the genomic DNA is analyzed using a nucleic acid sequencing assay.

50. The method of claim 41, wherein the gene is two or more genes.

51. The method of claim 41, wherein the gene is three or more genes.

52. The method of claim 41, wherein the mutation is two or more mutations.

53. The method of claim 41, wherein the mutation is three or more mutations.

54. A method, comprising:
a) analyzing genomic DNA from a canine subject for the presence of the DLA haplotype DLA-BRB1*00601, DQA1*005011, and DQB1*02001; and
b) identifying a canine subject having the DLA haplotype as a subject at elevated risk of developing speckled ANA-positive IMRD or having undiagnosed speckled ANA-positive IMRD.
